(19) United States
(12) Patent Application Publication
(10) Pub. No.: US 2026/0365720 A1
(43) Pub. Date: Nov. 19, 2026

(54) ANTI-LAYN ANTIBODIES AND USES THEREOF

(71) Applicants: Boehringer Ingelheim International GmbH, Ingelheim am Rhein (DE); Checkmab S.r.l., Milan (IT)

(72) Inventors: Katja FIEDLER, Mittelbiberach (DE); Tiziano DONNARUMMA, Milano (IT); Gerhard FISCHER, Vienna (AT); Simon FISCHER, Staig (DE); Renata GRIFANTINI, Siena (IT); Priyanka GUPTA, Newtown, CT (US); Manuele MARTINELLI, Arezzo (IT); Claudia MENDLER, Ulm (DE); Noah PEFAUR, New Milford, CT (US); Luca SORRENTINO, Milano (IT); Elena ZAGATO, Milano (IT)

(21) Appl. No.: 19/548,256

(22) Filed: Feb. 24, 2026

(30) Foreign Application Priority Data
Feb. 26, 2025 (EP) 25425006.1

(51) Int. Cl. C07K 16/18; A61K 45/06; A61P 35/00

(52) U.S. Cl. CPC C07K 16/18; A61K 45/06; A61P 35/00

(57) ABSTRACT

The present invention relates to anti-LAYN antibodies and antigen-binding fragments thereof, in particular new humanized anti-LAYN antibodies, and antigen-binding fragments thereof, and therapeutic and diagnostic methods and compositions for using the same.

### RELATED APPLICATION DISCLOSURE

This application claims the benefit under 35 U.S.C. § 119 of Application No. EP25425006.1 filed Feb. 26, 2025, which is hereby incorporated by reference herein in its entirety.

### SEQUENCE DISCLOSURE

This application includes, as part of its disclosure, a “Sequence Listing XML” pursuant to 37 C.F.R. § 1.831 (a) which is submitted in XML file format in a file named “01-3625-US-1.xml”, created on Feb. 23, 2026, and having a size of 310,361 bytes, which is hereby incorporated by reference herein in its entirety.

### FIELD

The disclosure generally relates to the field of anti-LAYN (anti-layilin) antibodies. More specifically, the disclosure relates to anti-LAYN antibodies or antigen-binding fragments thereof and to their medical use, for example, for the treatment of cancer. Pharmaceutical compositions and kits comprising the anti-LAYN antibodies or antigen-binding fragments thereof are also disclosed.

### BACKGROUND

Despite the progress in cancer immunotherapies, cancer remains a leading cause of death and one of the largest health problems worldwide with an estimated 20 million new cancer cases and 9.7 deaths in 2022. For 2050, more than 35 million new cancer cases are predicted, a 77% increase from the estimated 20 million cases in 2022.

The efficacy of cancer immunotherapies is often diminished by the tumor's ability to escape the immune response. In the tumor microenvironment tumor-infiltrating regulatory T lymphocytes (Tregs) can suppress anti-cancer immune responses. To overcome this hurdle and to unleash the immune response against the tumor, there is a need for agents suppressing or depleting intra-tumoral Tregs.

LAYN (also referred to as “layilin”) has been described to be upregulated in intra-tumoral Tregs and high expression of LAYN has been reported to correlate with poor prognosis in cancer patients (De Simone et al., 2016, Immunity, 45(5): 1135-1147; WO 2017/198631). LAYN is a type-I transmembrane protein that features a C-type lectin domain with two Ca²⁺ binding motifs. The extracellular domain (ECD) of LAYN interacts with collagen IV, a component of the extracellular matrix. On the other hand, its intracellular domain interacts with cytoskeletal-membrane linker molecules, which are associated with cell migration.

LAYN is expressed in various isoforms, with LAYN isoform 2 being the most expressed on tumor-infiltrating regulatory T cells (Tregs). In general, Tregs play a pivotal role in maintaining immune homeostasis by suppressing excessive immune responses. The high specificity of LAYN for intra-tumoral Tregs allows for the specific targeting of these cells within the tumor environment, sparing peripheral regulatory T cells.

Anti-LAYN antibodies are known in the art. For example, WO 2022/246085 and Glasgow et al., 2022 (Glasgow J E, Byrnes J R, Barbee S D, Moreau J M, Rosenblum M D, Wells J A. Identifying and antagonizing the interactions between layilin and glycosylated collagens. Cell Chem Biol. 2022 Apr. 21; 29(4):597-604.e7. doi: 10.1016/j.chembiol.2022.01.003. Epub 2022 Feb. 1) disclose anti-LAYN antibodies, including an antibody which interferes with binding between the ECD of LAYN and collagen IV.

There is a need for further anti-LAYN antibodies that specifically bind to Tregs, in particular intra-tumoral Tregs. In particular, there is a need for anti-LAYN antibodies showing a high antibody-dependent cellular cytotoxicity (ADCC) potential on LAYN-expressing cells (to mediate depletion of LAYN-expressing cells, in particular intra-tumoral Tregs). In addition, there is a need for anti-LAYN antibodies showing high binding affinity to the extracellular domain of human LAYN isoform 2 and an advantageous stability profile. Furthermore, there is a need for anti-LAYN antibodies showing efficacy for the treatment of cancer and/or having an acceptable safety/tolerability profile. There is also a need for anti-LAYN antibodies that do not interfere with binding of LAYN to collagen IV.

### SUMMARY

The present disclosure addresses the above need and others by providing agents, in particular antibodies or antigen-binding fragments thereof, which (specifically) bind to LAYN. In particular, the antibodies or antigen-binding fragments thereof, as disclosed herein, specifically bind to human LAYN (also referred to as “hLAYN” or “huLAYN”), in particular to isoform 1 and/or 2 of human LAYN, such as full length hLAYN isoform 2 (e.g., having the amino acid sequence of SEQ ID NO: 194). More specifically, the antibodies, or antigen-binding fragments thereof, as disclosed herein may (specifically) bind to the extracellular domain (ECD) of human LAYN isoform 2 (e.g., having the amino acid sequence of SEQ ID NO: 191 or 190) and/or to the extracellular domain (ECD) of human LAYN isoform 1 (e.g., having the amino acid sequence of SEQ ID NO: 197 or 196).

In some aspects, the present disclosure provides an antibody, or an antigen-binding fragment thereof, comprising any one of the sequences (SEQ ID NOs) described in Table 2 or a sequence variant thereof having at least 80% sequence identity (preferably at least 85%, more preferably at least 90%, even more preferably at least 95%, still more preferably at least 97%, particularly preferably at least 99% sequence identity). In some embodiments, such an antibody, or antigen-binding fragment thereof, comprises a set of six CDRs having the amino acid sequences of the six CDRs of any one of antibodies A1-A104 or P, as shown in Table 2, or in any one of Tables 3-8 below.

In particular, the present invention provides the following particular aspects and embodiments (“items”):
1. An anti-LAYN antibody, or an antigen-binding fragment thereof, which binds to an epitope of the extracellular domain of human LAYN isoform 2, wherein the epitope comprises (i) amino acid residues comprised in the loop of human LAYN isoform 2 containing residues 123-138 of the amino acid sequence as set forth in SEQ ID NO: 191, and (ii) amino acid residues comprised in the helix of human LAYN isoform 2 containing residues 52-67 of the amino acid sequence as set forth in SEQ ID NO: 191.
2. The anti-LAYN antibody, or an antigen-binding fragment thereof, according to item 1, wherein the epitope comprises (i) amino acid residues H123, P125 and A127 of the amino acid sequence as set forth in SEQ ID NO: 191, and (ii) amino acid residue E63 of the amino acid sequence as set forth in SEQ ID NO: 191.
3. The anti-LAYN antibody, or an antigen-binding fragment thereof, according to item 1 or 2, wherein the epitope comprises amino acid residues E52, K56, E59, 162, E63, L66, P67, V108, H123, Q124, P125, S126, A127, P128, A129, I131, G132, G133, P134, Y135, M136, F137 and Q138 of the amino acid sequence as set forth in SEQ ID NO: 191.
4. The anti-LAYN antibody, or an antigen-binding fragment thereof, according to any one of the previous items, wherein the antibody or the antigen-binding fragment specifically binds to human LAYN, in particular human LAYN isoform 2, at pH 6.
5. An anti-LAYN antibody, or an antigen-binding fragment thereof, specifically binding to human LAYN, in particular human LAYN isoform 2, at pH 6.
6. The anti-LAYN antibody, or an antigen-binding fragment thereof, according to item 4 or 5, wherein the human LAYN comprises (or consists of) the amino acid sequence of SEQ ID NO: 190 or SEQ ID NO: 191.
7. The anti-LAYN antibody, or an antigen-binding fragment thereof, according to any one of items 4 to 6, wherein the antibody or the antigen-binding fragment specifically binds to human LAYN after storage (of the antibody or the antigen-binding fragment) for at least four weeks at a temperature of 40 °C and/or at a concentration of the antibody or the antigen-binding fragment of 150 g/l.
8. The anti-LAYN antibody, or an antigen-binding fragment thereof, according to any one of the previous items, wherein the Fab fragment of the antibody, or the antigen-binding fragment, has a melting temperature Tm or an inflection point (IP) of a thermal transition curve of at least 80 °C.
9. An anti-LAYN antibody, or an antigen-binding fragment thereof, specifically binding to human LAYN, in particular human LAYN isoform 2 and/or human LAYN isoform 1, wherein the Fab fragment of the antibody, or the antigen-binding fragment, has a melting temperature Tm or an inflection point (IP) of a thermal transition curve of at least 80 °C.
10. The anti-LAYN antibody, or an antigen-binding fragment thereof, according to any one of the previous items, wherein the antibody or the antigen-binding fragment binds to human regulatory T cells and/or mediates depletion of human regulatory T cells.
11. The anti-LAYN antibody, or an antigen-binding fragment thereof, according to any one of the previous items, wherein the antibody or the antigen-binding fragment comprises:
   a heavy chain variable region comprising:
      a heavy chain complementary determining region 1 (HCDR1) having the amino acid sequence of X₁YWMD (SEQ ID NO: 206), wherein
         X₁ is preferably S or G, more preferably S;
      a heavy chain complementary determining region 2 (HCDR2) having the amino acid sequence of NIX₂PX₃SGX₄TNYX₅X₆KFX₇X₈ (SEQ ID NO: 207), wherein
         X₂ is preferably Y or N, more preferably Y;
         X₃ is preferably S or G, more preferably S;
         X₄ is preferably T or S, more preferably T,
         X₅ is preferably A or D, more preferably A;
         X₆ is preferably Q or E, more preferably Q;
         X₇ is preferably Q or K, more preferably Q; and
         Xe is preferably G or S, more preferably G; and
      a heavy chain complementary determining region 3 (HCDR3) having the amino acid sequence of X₉X₁₀YX₁₁RX₁₂AX₁₃X₁₄X₁₅ (SEQ ID NO: 208), wherein
         X₉ is preferably E or A, more preferably E;
         X₁₀ is preferably R, K or A, more preferably R;
         X₁₁ is preferably G or A, more preferably G;
         X₁₂ is preferably N or A, more preferably N;
         X₁₃ is preferably A or M, more preferably A;
         X₁₄ is preferably E, D or A, more preferably E; and
         X₁₅ is preferably Y or A, more preferably Y; and
   a light chain variable region comprising
      a light chain complementary determining region 1 (LCDR1) having the amino acid sequence of X₁₆ASQX₁₇₁X₁₈X₁₉X₂₀LA (SEQ ID NO: 209), wherein
         X₁₆ is preferably R or L, more preferably R;
         X₁₇ is preferably G, T or S, more preferably G;
         X₁₈ is preferably G or S, more preferably G;
         X₁₉ is preferably S or T, more preferably S; and
         X₂₀ is preferably W or Y, more preferably W;
      a light chain complementary determining region 2 (LCDR2) having the amino acid sequence of AAX₂₁SLX₂₂X₂₃ (SEQ ID NO: 210), wherein
         X₂₁ is preferably T or S, more preferably T;
         X₂₂ is preferably A or Q, more preferably A; and
         X₂₃ is preferably D, T, S or E, more preferably D; and
      a light chain complementary determining region 3 (LCDR3) having the amino acid sequence of QQX₂₄YSX₂₅PLT (SEQ ID NO: 211), wherein
         X₂₄ is L, A or S, preferably L; and
         X₂₅ is preferably T or F, more preferably T.
12. An anti-LAYN antibody, or an antigen-binding fragment thereof, comprising:
   a heavy chain variable region comprising:
      a heavy chain complementary determining region 1 (HCDR1) having the amino acid sequence of X₁YWMD (SEQ ID NO: 206), wherein
         X₁ is preferably S or G, more preferably S;
      a heavy chain complementary determining region 2 (HCDR2) having the amino acid sequence of NIX₂PX₃SGX₄TNYX₅X₆KFX₇X₈ (SEQ ID NO: 207), wherein
         X₂ is preferably Y or N, more preferably Y;
         X₃ is preferably S or G, more preferably S;
         X₄ is preferably T or S, more preferably T,
         X₅ is preferably A or D, more preferably A;
         X₆ is preferably Q or E, more preferably Q;
         X₇ is preferably Q or K, more preferably Q; and
         X₈ is preferably G or S, more preferably G; and
      a heavy chain complementary determining region 3 (HCDR3) having the amino acid sequence of X₉X₁₀YX₁₁RX₁₂AX₁₃X₁₄X₁₅ (SEQ ID NO: 208), wherein
         X₉ is preferably E or A, more preferably E;
         X₁₀ is preferably R, K or A, more preferably R;
         X₁₁ is preferably G or A, more preferably G;
         X₁₂ is preferably N or A, more preferably N;
         X₁₃ is preferably A or M, more preferably A;
         X₁₄ is preferably E, D or A, more preferably E; and
         X₁₅ is preferably Y or A, more preferably Y; and
   a light chain variable region comprising
      a light chain complementary determining region 1 (LCDR1) having the amino acid sequence of X₁₆ASQX₁₇₁X₁₈X₁₉X₂₀LA (SEQ ID NO: 209), wherein
         X₁₆ is preferably R or L, more preferably R;
         X₁₇ is preferably G, T or S, more preferably G;
         X₁₈ is preferably G or S, more preferably G;
         X₁₉ is preferably S or T, more preferably S; and
         X₂₀ is preferably W or Y, more preferably W;
      a light chain complementary determining region 2 (LCDR2) having the amino acid sequence of AAX₂₁SLX₂₂X₂₃ (SEQ ID NO: 210), wherein
         X₂₁ is preferably T or S, more preferably T;
         X₂ is preferably A or Q, more preferably A; and
         X₂₃ is preferably D, T, S or E, more preferably D; and
      a light chain complementary determining region 3 (LCDR3) having the amino acid sequence of QQX₂₄YSX₂₅PLT (SEQ ID NO: 211), wherein
         X₂₄ is L, A or S, preferably L; and
         X₂₅ is preferably T or F, more preferably T.
13. The anti-LAYN antibody, or an antigen-binding fragment thereof, according to any one of the previous items, wherein the antibody or the antigen-binding fragment comprises: a heavy chain variable region comprising an HCDR1 having the amino acid sequence of any one of SEQ ID NOs 1 or 131, an HCDR2 having the amino acid sequence of any one of SEQ ID NOs 2, 11, 19, 125, 128 or 168, and an HCDR3 having the amino acid sequence of any one of SEQ ID NOs 3, 29, 32, 77, 82, 85, 88, 91, 94, 142 or 183; and a light chain variable region comprising an LCDR1 having the amino acid sequence of any one of SEQ ID NOs 4, 12, 24, 58, 61, 64, 67 or 70, an LCDR2 having the amino acid sequence of any one of SEQ ID NOs 5, 44, 47, 50, 53 or 178, and an LCDR3 having the amino acid sequence of any one of SEQ ID NOs 6, 33, 38 or 41.
14. The anti-LAYN antibody, or an antigen-binding fragment thereof, according to any one of the previous items, wherein the antibody or the antigen-binding fragment comprises: a heavy chain variable region comprising an HCDR1 having the amino acid sequence of any one of SEQ ID NOs 1 or 131, an HCDR2 having the amino acid sequence of any one of SEQ ID NOs 2, 11, 19, 125, 128 or 168, and an HCDR3 having the amino acid sequence of any one of SEQ ID NOs 3, 29, 32, 77, 82, 85, 88, 91, 94 or 142; and a light chain variable region comprising an LCDR1 having the amino acid sequence of any one of SEQ ID NOs 4, 12, 24, 58, 61, 64, 67 or 70, an LCDR2 having the amino acid sequence of any one of SEQ ID NOs 5, 44, 47, 50, 53 or 178, and an LCDR3 having the amino acid sequence of any one of SEQ ID NOs 6, 33, 38 or 41.
15. The anti-LAYN antibody, or an antigen-binding fragment thereof, according to any one of the previous items, wherein the antibody or the antigen-binding fragment comprises:
   (i) a heavy chain variable region comprising an HCDR1 having the amino acid sequence of SEQ ID NO: 1, an HCDR2 having the amino acid sequence of SEQ ID NO: 2, and an HCDR3 having the amino acid sequence of SEQ ID NO: 3; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
   (ii) a heavy chain variable region comprising an HCDR1 having the amino acid sequence of SEQ ID NO: 1, an HCDR2 having the amino acid sequence of SEQ ID NO: 11, and an HCDR3 having the amino acid sequence of SEQ ID NO: 3; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6; or
   (iii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 3; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6; or
   (iv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 3; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
   (v) a heavy chain variable region comprising an HCDR1 having the amino acid sequence of SEQ ID NO: 1, an HCDR2 having the amino acid sequence of SEQ ID NO: 11, and an HCDR3 having the amino acid sequence of SEQ ID NO: 3; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (vi) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 29; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
   (vii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 29; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
   (viii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 29; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6; or
   (ix) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 33; or
   (x) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 38; or
   (xi) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 41; or
   (xii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xiii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 44, and an LCDR3 having the amino acid sequence of SEQ ID NO: 6; or
   (xiv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 47, and an LCDR3 having the amino acid sequence of SEQ ID NO: 6; or
   (xv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 50, and an LCDR3 having the amino acid sequence of SEQ ID NO: 6; or
   (xvi) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 53, and an LCDR3 having the amino acid sequence of SEQ ID NO: 6; or
   (xvii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 58, an LCDR2 having the amino acid sequence of SEQ ID NO: 5, and an LCDR3 having the amino acid sequence of SEQ ID NO: 6; or
   (xviii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 61, an LCDR2 having the amino acid sequence of SEQ ID NO: 5, and an LCDR3 having the amino acid sequence of SEQ ID NO: 6; or
   (xix) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 64, an LCDR2 having the amino acid sequence of SEQ ID NO: 5, and an LCDR3 having the amino acid sequence of SEQ ID NO: 6; or
   (xx) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 67, an LCDR2 having the amino acid sequence of SEQ ID NO: 5, and an LCDR3 having the amino acid sequence of SEQ ID NO: 6; or (xxi) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 70, an LCDR2 having the amino acid sequence of SEQ ID NO: 5, and an LCDR3 having the amino acid sequence of SEQ ID NO: 6; or
   (xxii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6; or
   (xxiii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 77; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xxiv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 82; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xxv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 3; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xxvi) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 85; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xxvii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 88; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xxviii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 91; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xxix) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 94; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xxx) a heavy chain variable region comprising an HCDR1 having the amino acid sequence of SEQ ID NO: 1, an HCDR2 having the amino acid sequence of SEQ ID NO: 11, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xxxi) a heavy chain variable region comprising an HCDR1 having the amino acid sequence of SEQ ID NO: 1, an HCDR2 having the amino acid sequence of SEQ ID NO: 125, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xxxii) a heavy chain variable region comprising an HCDR1 having the amino acid sequence of SEQ ID NO: 1, an HCDR2 having the amino acid sequence of SEQ ID NO: 128, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xxxiii) a heavy chain variable region comprising an HCDR1 having the amino acid sequence of SEQ ID NO: 131, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xxxiv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
   (xxxv) a heavy chain variable region comprising an HCDR1 having the amino acid sequence of SEQ ID NO: 1, an HCDR2 having the amino acid sequence of SEQ ID NO: 2, and an HCDR3 having the amino acid sequence of SEQ ID NO: 3; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xxxvi) a heavy chain variable region comprising an HCDR1 having the amino acid sequence of SEQ ID NO: 1, an HCDR2 having the amino acid sequence of SEQ ID NO: 2, and an HCDR3 having the amino acid sequence of SEQ ID NO: 3; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6; or
   (xxxvii) a heavy chain variable region comprising an HCDR1 having the amino acid sequence of SEQ ID NO: 1, an HCDR2 having the amino acid sequence of SEQ ID NO: 125, and an HCDR3 having the amino acid sequence of SEQ ID NO: 3; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xxxviii) a heavy chain variable region comprising an HCDR1 having the amino acid sequence of SEQ ID NO: 1, an HCDR2 having the amino acid sequence of SEQ ID NO: 125, and an HCDR3 having the amino acid sequence of SEQ ID NO: 3; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
   (xxxix) a heavy chain variable region comprising an HCDR1 having the amino acid sequence of SEQ ID NO: 1, an HCDR2 having the amino acid sequence of SEQ ID NO: 125, and an HCDR3 having the amino acid sequence of SEQ ID NO: 3; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6; or
   (xl) a heavy chain variable region comprising an HCDR1 having the amino acid sequence of SEQ ID NO: 1, an HCDR2 having the amino acid sequence of SEQ ID NO: 11, and an HCDR3 having the amino acid sequence of SEQ ID NO: 3; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
   (xli) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 29; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xlii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 142; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xliii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 142; and a light chain variable region comprising an LCDR1 having the amino acid sequence of SEQ ID NO: 24, an LCDR2 having the amino acid sequence of SEQ ID NO: 178, and an LCDR3 having the amino acid sequence of SEQ ID NO: 6; or
   (xliv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 183; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
   (xlv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 183; and a light chain variable region comprising an LCDR1 having the amino acid sequence of SEQ ID NO: 24, an LCDR2 having the amino acid sequence of SEQ ID NO: 178, and an LCDR3 having the amino acid sequence of SEQ ID NO: 6; or
   (xlvi) a heavy chain variable region comprising an HCDR1 having the amino acid sequence of SEQ ID NO: 1, an HCDR2 having the amino acid sequence of SEQ ID NO: 168, and an HCDR3 having the amino acid sequence of SEQ ID NO: 142; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6.
16. The anti-LAYN antibody, or an antigen-binding fragment thereof, according to any one of the previous items, wherein the antibody or the antigen-binding fragment comprises:
   a heavy chain variable region (VH) comprising the amino acid sequence of QVQLX₂₆QX₂₇GX₂₈EX₂₉X₃₀X₃₁PGASVKX₃₂SCKASGYTFTX₁YWMDWVX₃₃QX₃₄PG X₃₅GLEWX₃₆GNIX₂PX₃SGX₄TNYX₅X₆KFX₇X₈X₃₇X₃₈TX₃₉TX₄₀DX₄₁X₄₂X₄₃X₄₄TX₄₅YM X₄₆LSX₄₇LX₄₈SX₄₉DX₅₀AX₅₁YYCX₅₂RX₉X₁₀YX₁₁RX₁₂AX₁₃X₁₄X₁₅WGQGTX₅₃VTVSS
   (SEQ ID NO: 212), wherein
      X₁ is preferably S or G, more preferably S,
      X₂ is preferably Y or N, more preferably Y,
      X₃ is preferably G or S, more preferably S,
      X₄ is preferably S or T, more preferably T,
      X₅ is preferably D or A, more preferably A,
      X₆ is preferably E or Q, more preferably Q,
      X₇ is preferably K or Q, more preferably Q,
      X₈ is preferably S or G, more preferably G,
      X₉ is preferably E or A, more preferably E,
      X₁₀ is preferably R, K or A, more preferably R,
      X₁₁ is preferably G or A, more preferably G,
      X₁₂ is preferably N or A, more preferably N,
      X₁₃ is preferably M or A, more preferably A,
      X₁₄ is preferably D, E or A, more preferably E,
      X₁₅ is preferably Y or A, more preferably Y,
      X₂₆ is preferably V or Q, more preferably V,
      X₂₇ is preferably S or P, more preferably S,
      X₂₈ is preferably A, S or T, more preferably A,
      X₂₉ is preferably V or L, more preferably V,
      X₃₀ is preferably K or V, more preferably K,
      X₃₁ is preferably K or R, more preferably K,
      X₃₂ is preferably V or L, more preferably V,
      X₃₃ is preferably R or K, more preferably R,
      X₃₄ is preferably A or R, more preferably A,
      X₃₅ is preferably Q or K, more preferably Q,
      X₃₆ is preferably M or I, more preferably M,
      X₃₇ is preferably R or K, more preferably R,
      X₃₈ is preferably V or A, more preferably V,
      X₃₉ is preferably M, L or I, more preferably M,
      X₄₀ is preferably V, A or R, more preferably V,
      X₄₁ is preferably K or T, more preferably K,
      X₄₂ is preferably S or A, more preferably S,
      X₄₃ is preferably T, K, I or S, more preferably T,
      X₄₄ is preferably S or D, more preferably S,
      X₄₅ is preferably A, L or V, more preferably A,
      X₄₆ is preferably E or Q, more preferably E,
      X₄₇ is preferably S or R, more preferably S,
      X₄₈ is preferably R or T, more preferably R,
      X₄₉ is preferably E or D, more preferably E,
      X₅₀ is preferably T or S, more preferably T,
      X₅₁ is preferably V or T, more preferably V,
      X₅₂ is preferably A or T, more preferably A, and
      X₅₃ is preferably L or S, more preferably L; and
   a light chain variable region (VL) comprising the amino acid sequence of DIQMTQSPX₅₄SX₅₅SASX₅₆GX₅₇X₅₈VTITCX₁₆ASQX₁₇₁X₁₈X₁₉X₂₀LAWYQQKPGKX₅₉PX₆₀LLIYAAX₂₁SLX₂₂X₂₃GVPSRFSGSGSGTX₆₁FX₆₂X₆₃X₆₄ISSLQX₆₅EDFX₆₆X₆₇YY CQQX₂₄YSX₂₅PLTFGGGTKLEIK
   (SEQ ID NO: 213), wherein
      X₁₆ is preferably L or R, more preferably R,
      X₁₇ is preferably T, G or S, more preferably G,
      X₁₈ is preferably G or S, more preferably G,
      X₁₉ is preferably T or S, more preferably S,
      X₂₀ is preferably W or Y, more preferably W,
      X₂₁ is preferably T or S, more preferably T,
      X₂₂ is preferably A or Q, more preferably A,
      X₂₃ is preferably D, T, S or E, more preferably D,
      X₂₄ is L, A or S, preferably L,
      X₂₅ is preferably T or F, more preferably T,
      X₅₄ is preferably S or A, more preferably S,
      X₅₅ is preferably V, L or Q, more preferably V,
      X₅₆ is preferably V or L, more preferably V,
      X₅₇ is preferably D or E, more preferably D,
      X₅₈ is preferably R or S, more preferably R,
      X₅₉ is preferably A or S, more preferably A,
      X₆₀ is preferably K or Q, more preferably K,
      X₆₁ is preferably D or K, more preferably D,
      X₆₂ is preferably T or S, more preferably T,
      X₆₃ is preferably L or F, more preferably L,
      X₆₄ is preferably T or K, more preferably T,
      X₆₅ is preferably P or A, more preferably P,
      X₆ is preferably A or V, more preferably A, and
      X₆₇ is preferably T or S, more preferably T.
17. An anti-LAYN antibody, or an antigen-binding fragment thereof, comprising:
   a heavy chain variable region (VH) comprising the amino acid sequence of QVQLX₂₆QX₂₇GX₂₈EX₂₉X₃₀X₃₁PGASVKX₃₂SCKASGYTFTX₁YWMDWVX₃₃QX₃₄PG X₃₅GLEWX₃₆GNIX₂PX₃SGX₄TNYX₅X₆KFX₇X₈X₃₇X₃₈TX₃₉TX₄₀DX₄₁X₄₂X₄₃X₄₄TX₄₅YM X₄₆LSX₄₇LX₄₈SX₄₉DX₅₀AX₅₁YYCX₅₂RX₉X₁₀YX₁₁RX₁₂AX₁₃X₁₄X₁₅WGQGTX₅₃VTVSS
   (SEQ ID NO: 212), wherein
      X₁ is preferably S or G, more preferably S,
      X₂ is preferably Y or N, more preferably Y,
      X₃ is preferably G or S, more preferably S,
      X₄ is preferably S or T, more preferably T,
      X₅ is preferably D or A, more preferably A,
      X₆ is preferably E or Q, more preferably Q,
      X₇ is preferably K or Q, more preferably Q,
      Xe is preferably S or G, more preferably G,
      X₉ is preferably E or A, more preferably E,
      X₁₀ is preferably R, K or A, more preferably R,
      X₁₁ is preferably G or A, more preferably G,
      X₁₂ is preferably N or A, more preferably N,
      X₁₃ is preferably M or A, more preferably A,
      X₁₄ is preferably D, E or A, more preferably E,
      X₁₅ is preferably Y or A, more preferably Y,
      X₂₆ is preferably V or Q, more preferably V,
      X₂₇ is preferably S or P, more preferably S,
      X₂₈ is preferably A, S or T, more preferably A,
      X₂₉ is preferably V or L, more preferably V,
      X₃₀ is preferably K or V, more preferably K,
      X₃₁ is preferably K or R, more preferably K,
      X₃₂ is preferably V or L, more preferably V,
      X₃₃ is preferably R or K, more preferably R,
      X₃₄ is preferably A or R, more preferably A,
      X₃₅ is preferably Q or K, more preferably Q,
      X₃₆ is preferably M or I, more preferably M,
      X₃₇ is preferably R or K, more preferably R,
      X₃₈ is preferably V or A, more preferably V,
      X₃₉ is preferably M, L or I, more preferably M,
      X₄₀ is preferably V, A or R, more preferably V,
      X₄₁ is preferably K or T, more preferably K,
      X₄₂ is preferably S or A, more preferably S,
      X₄₃ is preferably T, K, I or S, more preferably T,
      X₄₄ is preferably S or D, more preferably S,
      X₄₅ is preferably A, L or V, more preferably A,
      X₄₆ is preferably E or Q, more preferably E,
      X₄₇ is preferably S or R, more preferably S,
      X₄₈ is preferably R or T, more preferably R,
      X₄g is preferably E or D, more preferably E,
      X₅₀ is preferably T or S, more preferably T,
      X₅₁ is preferably V or T, more preferably V,
      X₅₂ is preferably A or T, more preferably A, and
      X₅₃ is preferably L or S, more preferably L; and
   a light chain variable region (VL) comprising the amino acid sequence of DIQMTQSPX₅₄SX₅₅SASX₅₆GX₅₇X₅₈VTITCX₁₆ASQX₁₇₁X₁₈X₁₉X₂₀LAWYQQKPGKX₅₉PX₆₀LLIYAAX₂₁SLX₂₂X₂₃GVPSRFSGSGSGTX₆₁FX₆₂X₆₃X₆₄ISSLQX₆₅EDFX₆X₆₇YY CQQX₂₄YSX₂₅PLTFGGGTKLEIK
   (SEQ ID NO: 213), wherein
      X₁₆ is preferably L or R, more preferably R,
      X₁₇ is preferably T, G or S, more preferably G,
      X₁₈ is preferably G or S, more preferably G,
      X₁₉ is preferably T or S, more preferably S,
      X₂₀ is preferably W or Y, more preferably W,
      X₂₁ is preferably T or S, more preferably T,
      X₂₂ is preferably A or Q, more preferably A,
      X₂₃ is preferably D, T, S or E, more preferably D,
      X₂₄ is L, A or S, preferably L,
      X₂₅ is preferably T or F, more preferably T,
      X₅₄ is preferably S or A, more preferably S,
      X₅₅ is preferably V, L or Q, more preferably V,
      X₅₆ is preferably V or L, more preferably V,
      X₅₇ is preferably D or E, more preferably D,
      X₅₈ is preferably R or S, more preferably R,
      X₅₉ is preferably A or S, more preferably A,
      X₆₀ is preferably K or Q, more preferably K,
      X₆₁ is preferably D or K, more preferably D,
      X₆₂ is preferably T or S, more preferably T,
      X₆₃ is preferably L or F, more preferably L,
      X₆₄ is preferably T or K, more preferably T,
      X₆₅ is preferably P or A, more preferably P,
      X₆₆ is preferably A or V, more preferably A, and
      X₆₇ is preferably T or S, more preferably T.
18. The anti-LAYN antibody, or an antigen-binding fragment thereof, according to any one of the previous items, wherein the antibody or the antigen-binding fragment comprises: a heavy chain variable region comprising an amino acid sequence at least 80% identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188; and a light chain variable region comprising an amino acid sequence at least 80% identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179.
19. The anti-LAYN antibody, or an antigen-binding fragment thereof, according to any one of the previous items, wherein the antibody or the antigen-binding fragment comprises: a heavy chain variable region comprising an amino acid sequence at least 80% identical to the amino acid sequence of any one of SEQ ID NOs 7, 13, 17, 20, 22, 27, 30, 34, 78, comprising an amino acid sequence at least 80% identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179.
20. An anti-LAYN antibody, or an antigen-binding fragment thereof, comprising: a heavy chain variable region comprising an amino acid sequence at least 80% identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188; and a light chain variable region comprising an amino acid sequence at least 80% identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179.
21. An anti-LAYN antibody, or an antigen-binding fragment thereof, comprising: a heavy chain variable region comprising an amino acid sequence at least 80% identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181; and a light chain variable region comprising an amino acid sequence at least 80% identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179.
22. The anti-LAYN antibody, or an antigen-binding fragment thereof, according to any one of the previous items, wherein the antibody or the antigen-binding fragment comprises:
   (i) a heavy chain variable region comprising an amino acid sequence at least 80% identical to the amino acid sequence of SEQ ID NO: 7, and a light chain variable region comprising an amino acid sequence at least 80% identical to the amino acid sequence of SEQ ID NO: 8; or
   (ii) a heavy chain variable region comprising an amino acid sequence at least 80% identical to the amino acid sequence of SEQ ID NO: 13, and a light chain variable region comprising an amino acid sequence at least 80% identical to the amino acid sequence of SEQ ID NO: 14; or
   (iii) a heavy chain variable region comprising an amino acid sequence at least 80% identical to the amino acid sequence of SEQ ID NO: 17, and a light chain variable region comprising an amino acid sequence at least 80% identical to the amino acid sequence of SEQ ID NO: 8; or
   (iv) a heavy chain variable region comprising an amino acid sequence at least 80% identical to the amino acid sequence of SEQ ID NO: 20, and a light chain variable region comprising an amino acid sequence at least 80% identical to the amino acid sequence of SEQ ID NO: 14; or
   (v) a heavy chain variable region comprising an amino acid sequence at least 80% identical to the amino acid sequence of SEQ ID NO: 22, and a light chain variable region comprising an amino acid sequence at least 80% identical to the amino acid sequence of SEQ ID NO: 8; or
   (vi) a heavy chain variable region comprising an amino acid sequence at least 80% identical to the amino acid sequence of SEQ ID NO: 22, and a light chain variable region comprising an amino acid sequence at least 80% identical to the amino acid sequence of SEQ ID NO: 14; or
   (vii) a heavy chain variable region comprising an amino acid sequence at least 80% identical to the amino acid sequence of SEQ ID NO: 13, and a light chain variable region comprising an amino acid sequence at least 80% identical to the amino acid sequence of SEQ ID NO: 25; or
   (viii) a heavy chain variable region comprising an amino acid sequence at least 80% identical to the amino acid sequence of SEQ ID NO: 27, and a light chain variable region comprising an amino acid sequence at least 80% identical to the amino acid sequence of SEQ ID NO: 8; or
   (ix) a heavy chain variable region comprising an amino acid sequence at least 80% identical to the amino acid sequence of SEQ ID NO: 27, and a light chain variable region comprising an amino acid sequence at least 80% identical to the amino acid sequence of SEQ ID NO: 14; or
   (x) a heavy chain variable region comprising an amino acid sequence at least 80% identical to the amino acid sequence of SEQ ID NO: 30, and a light chain variable region comprising an amino acid sequence at least 80% identical to the amino acid sequence of SEQ ID NO: 8; or
   (xi) a heavy chain variable region comprising an amino acid sequence at least 80% identical to the amino acid sequence of SEQ ID NO: 30, and a light chain variable region comprising an amino acid sequence at least 80% identical to the amino acid sequence of SEQ ID NO: 14; or
   (xii) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 35; or
   (xiii) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 39; or
   (xiv) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 42; or
   (xv) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 25; or
   (xvi) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 45; or
   (xvii) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 48; or
   (xviii) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 51; or
   (xix) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 54; or
   (xx) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 56; or
   (xxi) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 59; or
   (xxii) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 62; or
   (xxiii) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 65; or
   (xxiv) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 68; or
   (xxv) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 71; or
   (xxvi) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 73; or
   (xxvii) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 75; or
   (xxviii) a heavy chain variable region comprising an amino acid sequence at least 80% identical to the amino acid sequence of SEQ ID NO: 78, and a light chain variable region comprising an amino acid sequence at least 80% identical to the amino acid sequence of SEQ ID NO: 79; or
   (xxix) a heavy chain variable region comprising an amino acid sequence at least 80% identical to the amino acid sequence of SEQ ID NO: 83, and a light chain variable region comprising an amino acid sequence at least 80% identical to the amino acid sequence of SEQ ID NO: 79; or
   (xxx) a heavy chain variable region comprising an amino acid sequence at least 80% identical to the amino acid sequence of SEQ ID NO: 27, and a light chain variable region comprising an amino acid sequence at least 80% identical to the amino acid sequence of SEQ ID NO: 79; or
   (xxxi) a heavy chain variable region comprising an amino acid sequence at least 80% identical to the amino acid sequence of SEQ ID NO: 86, and a light chain variable region comprising an amino acid sequence at least 80% identical to the amino acid sequence of SEQ ID NO: 79; or
   (xxxii) a heavy chain variable region comprising an amino acid sequence at least 80% identical to the amino acid sequence of SEQ ID NO: 89, and a light chain variable region comprising an amino acid sequence at least 80% identical to the amino acid sequence of SEQ ID NO: 79; or
   (xxxiii) a heavy chain variable region comprising an amino acid sequence at least 80% identical to the amino acid sequence of SEQ ID NO: 92, and a light chain variable region comprising an amino acid sequence at least 80% identical to the amino acid sequence of SEQ ID NO: 79; or
   (xxxiv) a heavy chain variable region comprising an amino acid sequence at least 80% identical to the amino acid sequence of SEQ ID NO: 95, and a light chain variable region comprising an amino acid sequence at least 80% identical to the amino acid sequence of SEQ ID NO: 79; or
   (xxxv) a heavy chain variable region comprising an amino acid sequence at least 80% identical to the amino acid sequence of SEQ ID NO: 97, and a light chain variable region comprising an amino acid sequence at least 80% identical to the amino acid sequence of SEQ ID NO: 79; or
   (xxxvi) a heavy chain variable region comprising an amino acid sequence at least 80% identical to the amino acid sequence of SEQ ID NO: 99, and a light chain variable region comprising an amino acid sequence at least 80% identical to the amino acid sequence of SEQ ID NO: 79; or
   (xxxvii) a heavy chain variable region comprising an amino acid sequence at least 80% identical to the amino acid sequence of SEQ ID NO: 101, and a light chain variable region comprising an amino acid sequence at least 80% identical to the amino acid sequence of SEQ ID NO: 79; or
   (xxxviii) a heavy chain variable region comprising an amino acid sequence at least 80% identical to the amino acid sequence of SEQ ID NO: 103, and a light chain variable region comprising an amino acid sequence at least 80% identical to the amino acid sequence of SEQ ID NO: 79; or
   (xxxix) a heavy chain variable region comprising an amino acid sequence at least 80% identical to the amino acid sequence of SEQ ID NO: 105, and a light chain variable region comprising an amino acid sequence at least 80% identical to the amino acid sequence of SEQ ID NO: 79; or
   (xl) a heavy chain variable region comprising an amino acid sequence at least 80% identical to the amino acid sequence of SEQ ID NO: 107, and a light chain variable region comprising an amino acid sequence at least 80% identical to the amino acid sequence of SEQ ID NO: 79; or
   (xli) a heavy chain variable region comprising an amino acid sequence at least 80% identical to the amino acid sequence of SEQ ID NO: 109, and a light chain variable region comprising an amino acid sequence at least 80% identical to the amino acid sequence of SEQ ID NO: 79; or
   (xlii) a heavy chain variable region comprising an amino acid sequence at least 80% identical to the amino acid sequence of SEQ ID NO: 111, and a light chain variable region comprising an amino acid sequence at least 80% identical to the amino acid sequence of SEQ ID NO: 79; or
   (xliii) a heavy chain variable region comprising an amino acid sequence at least 80% identical to the amino acid sequence of SEQ ID NO: 113, and a light chain variable region comprising an amino acid sequence at least 80% identical to the amino acid sequence of SEQ ID NO: 79; or
   (xliv) a heavy chain variable region comprising an amino acid sequence at least 80% identical to the amino acid sequence of SEQ ID NO: 115, and a light chain variable region comprising an amino acid sequence at least 80% identical to the amino acid sequence of SEQ ID NO: 79; or
   (xlv) a heavy chain variable region comprising an amino acid sequence at least 80% identical to the amino acid sequence of SEQ ID NO: 117, and a light chain variable region comprising an amino acid sequence at least 80% identical to the amino acid sequence of SEQ ID NO: 79; or
   (xlvi) a heavy chain variable region comprising an amino acid sequence at least 80% identical to the amino acid sequence of SEQ ID NO: 119, and a light chain variable region comprising an amino acid sequence at least 80% identical to the amino acid sequence of SEQ ID NO: 79; or
   (xlvii) a heavy chain variable region comprising an amino acid sequence at least 80% identical to the amino acid sequence of SEQ ID NO: 121, and a light chain variable region comprising an amino acid sequence at least 80% identical to the amino acid sequence of SEQ ID NO: 79; or
   (xlviii) a heavy chain variable region comprising an amino acid sequence at least 80% identical to the amino acid sequence of SEQ ID NO: 123, and a light chain variable region comprising an amino acid sequence at least 80% identical to the amino acid sequence of SEQ ID NO: 79; or
   (xlix) a heavy chain variable region comprising an amino acid sequence at least 80% identical to the amino acid sequence of SEQ ID NO: 126, and a light chain variable region comprising an amino acid sequence at least 80% identical to the amino acid sequence of SEQ ID NO: 79; or
   (l) a heavy chain variable region comprising an amino acid sequence at least 80% identical to the amino acid sequence of SEQ ID NO: 129, and a light chain variable region comprising an amino acid sequence at least 80% identical to the amino acid sequence of SEQ ID NO: 79; or
   (li) a heavy chain variable region comprising an amino acid sequence at least 80% identical to the amino acid sequence of SEQ ID NO: 132, and a light chain variable region comprising an amino acid sequence at least 80% identical to the amino acid sequence of SEQ ID NO: 79; or
   (lii) a heavy chain variable region comprising an amino acid sequence at least 80% identical to the amino acid sequence of SEQ ID NO: 134, and a light chain variable region comprising an amino acid sequence at least 80% identical to the amino acid sequence of SEQ ID NO: 79; or
   (liii) a heavy chain variable region comprising an amino acid sequence at least 80% identical to the amino acid sequence of SEQ ID NO: 136, and a light chain variable region comprising an amino acid sequence at least 80% identical to the amino acid sequence of SEQ ID NO: 79; or
   (liv) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 8; or
   (lv) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 14; or
   (lvi) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 138; or
   (lvii) a heavy chain variable region comprising an amino acid sequence at least 80% identical to the amino acid sequence of SEQ ID NO: 17, and a light chain variable region comprising an amino acid sequence at least 80% identical to the amino acid sequence of SEQ ID NO: 79; or
   (lviii) a heavy chain variable region comprising an amino acid sequence at least 80% identical to the amino acid sequence of SEQ ID NO: 17, and a light chain variable region comprising an amino acid sequence at least 80% identical to the amino acid sequence of SEQ ID NO: 14; or
   (lix) a heavy chain variable region comprising an amino acid sequence at least 80% identical to the amino acid sequence of SEQ ID NO: 17, and a light chain variable region comprising an amino acid sequence at least 80% identical to the amino acid sequence of SEQ ID NO: 138; or
   (lx) a heavy chain variable region comprising an amino acid sequence at least 80% identical to the amino acid sequence of SEQ ID NO: 17, and a light chain variable region comprising an amino acid sequence at least 80% identical to the amino acid sequence of SEQ ID NO: 25; or
   (lxi) a heavy chain variable region comprising an amino acid sequence at least 80% identical to the amino acid sequence of SEQ ID NO: 7, and a light chain variable region comprising an amino acid sequence at least 80% identical to the amino acid sequence of SEQ ID NO: 79; or
   (lxii) a heavy chain variable region comprising an amino acid sequence at least 80% identical to the amino acid sequence of SEQ ID NO: 7, and a light chain variable region comprising an amino acid sequence at least 80% identical to the amino acid sequence of SEQ ID NO: 14; or
   (lxiii) a heavy chain variable region comprising an amino acid sequence at least 80% identical to the amino acid sequence of SEQ ID NO: 7, and a light chain variable region comprising an amino acid sequence at least 80% identical to the amino acid sequence of SEQ ID NO: 138; or
   (lxiv) a heavy chain variable region comprising an amino acid sequence at least 80% identical to the amino acid sequence of SEQ ID NO: 7, and a light chain variable region comprising an amino acid sequence at least 80% identical to the amino acid sequence of SEQ ID NO: 25; or
   (lxv) a heavy chain variable region comprising an amino acid sequence at least 80% identical to the amino acid sequence of SEQ ID NO: 140, and a light chain variable region comprising an amino acid sequence at least 80% identical to the amino acid sequence of SEQ ID NO: 79; or
   (lxvi) a heavy chain variable region comprising an amino acid sequence at least 80% identical to the amino acid sequence of SEQ ID NO: 140, and a light chain variable region comprising an amino acid sequence at least 80% identical to the amino acid sequence of SEQ ID NO: 8; or
   (lxvii) a heavy chain variable region comprising an amino acid sequence at least 80% identical to the amino acid sequence of SEQ ID NO: 140, and a light chain variable region comprising an amino acid sequence at least 80% identical to the amino acid sequence of SEQ ID NO: 14; or
   (lxviii) a heavy chain variable region comprising an amino acid sequence at least 80% identical to the amino acid sequence of SEQ ID NO: 140, and a light chain variable region comprising an amino acid sequence at least 80% identical to the amino acid sequence of SEQ ID NO: 138; or
   (lxix) a heavy chain variable region comprising an amino acid sequence at least 80% identical to the amino acid sequence of SEQ ID NO: 140, and a light chain variable region comprising an amino acid sequence at least 80% identical to the amino acid sequence of SEQ ID NO: 25; or
   (lxx) a heavy chain variable region comprising an amino acid sequence at least 80% identical to the amino acid sequence of SEQ ID NO: 20, and a light chain variable region comprising an amino acid sequence at least 80% identical to the amino acid sequence of SEQ ID NO: 79; or
   (lxxi) a heavy chain variable region comprising an amino acid sequence at least 80% identical to the amino acid sequence of SEQ ID NO: 20, and a light chain variable region comprising an amino acid sequence at least 80% identical to the amino acid sequence of SEQ ID NO: 8; or
   (lxxii) a heavy chain variable region comprising an amino acid sequence at least 80% identical to the amino acid sequence of SEQ ID NO: 20, and a light chain variable region comprising an amino acid sequence at least 80% identical to the amino acid sequence of SEQ ID NO: 138; or
   (lxxiii) a heavy chain variable region comprising an amino acid sequence at least 80% identical to the amino acid sequence of SEQ ID NO: 20, and a light chain variable region comprising an amino acid sequence at least 80% identical to the amino acid sequence of SEQ ID NO: 25; or
   (lxxiv) a heavy chain variable region comprising an amino acid sequence at least 80% identical to the amino acid sequence of SEQ ID NO: 22, and a light chain variable region comprising an amino acid sequence at least 80% identical to the amino acid sequence of SEQ ID NO: 79; or
   (lxxv) a heavy chain variable region comprising an amino acid sequence at least 80% identical to the amino acid sequence of SEQ ID NO: 22, and a light chain variable region comprising an amino acid sequence at least 80% identical to the amino acid sequence of SEQ ID NO: 138; or
   (lxxvi) a heavy chain variable region comprising an amino acid sequence at least 80% identical to the amino acid sequence of SEQ ID NO: 22, and a light chain variable region comprising an amino acid sequence at least 80% identical to the amino acid sequence of SEQ ID NO: 25; or
   (lxxvii) a heavy chain variable region comprising an amino acid sequence at least 80% identical to the amino acid sequence of SEQ ID NO: 13, and a light chain variable region comprising an amino acid sequence at least 80% identical to the amino acid sequence of SEQ ID NO: 79; or
   (lxxviii) a heavy chain variable region comprising an amino acid sequence at least 80% identical to the amino acid sequence of SEQ ID NO: 13, and a light chain variable region comprising an amino acid sequence at least 80% identical to the amino acid sequence of SEQ ID NO: 8; or
   (lxxix) a heavy chain variable region comprising an amino acid sequence at least 80% identical to the amino acid sequence of SEQ ID NO: 13, and a light chain variable region comprising an amino acid sequence at least 80% identical to the amino acid sequence of SEQ ID NO: 138; or
   (lxxx) a heavy chain variable region comprising an amino acid sequence at least 80% identical to the amino acid sequence of SEQ ID NO: 27, and a light chain variable region comprising an amino acid sequence at least 80% identical to the amino acid sequence of SEQ ID NO: 138; or
   (lxxxi) a heavy chain variable region comprising an amino acid sequence at least 80% identical to the amino acid sequence of SEQ ID NO: 27, and a light chain variable region comprising an amino acid sequence at least 80% identical to the amino acid sequence of SEQ ID NO: 25; or
   (lxxxii) a heavy chain variable region comprising an amino acid sequence at least 80% identical to the amino acid sequence of SEQ ID NO: 30, and a light chain variable region comprising an amino acid sequence at least 80% identical to the amino acid sequence of SEQ ID NO: 79; or
   (lxxxiii) a heavy chain variable region comprising an amino acid sequence at least 80% identical to the amino acid sequence of SEQ ID NO: 30, and a light chain variable region comprising an amino acid sequence at least 80% identical to the amino acid sequence of SEQ ID NO: 138; or
   (lxxxiv) a heavy chain variable region comprising an amino acid sequence at least 80% identical to the amino acid sequence of SEQ ID NO: 30, and a light chain variable region comprising an amino acid sequence at least 80% identical to the amino acid sequence of SEQ ID NO: 25; or
   (lxxxv) a heavy chain variable region comprising an amino acid sequence at least 80% identical to the amino acid sequence of SEQ ID NO: 143, and a light chain variable region comprising an amino acid sequence at least 80% identical to the amino acid sequence of SEQ ID NO: 79; or
   (lxxxvi) a heavy chain variable region comprising an amino acid sequence at least 80% identical to the amino acid sequence of SEQ ID NO: 34, and a light chain variable region comprising an amino acid sequence at least 80% identical to the amino acid sequence of SEQ ID NO: 79; or
   (lxxxvii) a heavy chain variable region comprising an amino acid sequence at least 80% identical to the amino acid sequence of SEQ ID NO: 145, and a light chain variable region comprising an amino acid sequence at least 80% identical to the amino acid sequence of SEQ ID NO: 79; or
   (lxxxviii) a heavy chain variable region comprising an amino acid sequence at least 80% identical to the amino acid sequence of SEQ ID NO: 181, and a light chain variable region comprising an amino acid sequence at least 80% identical to the amino acid sequence of SEQ ID NO: 79; or
   (lxxxix) a heavy chain variable region comprising an amino acid sequence at least 80% identical to the amino acid sequence of SEQ ID NO: 143, and a light chain variable region comprising an amino acid sequence at least 80% identical to the amino acid sequence of SEQ ID NO: 179; or
   (xc) a heavy chain variable region comprising an amino acid sequence at least 80% identical to the amino acid sequence of SEQ ID NO: 145, and a light chain variable region comprising an amino acid sequence at least 80% identical to the amino acid sequence of SEQ ID NO: 179; or
   (xci) a heavy chain variable region comprising an amino acid sequence at least 80% identical to the amino acid sequence of SEQ ID NO: 181, and a light chain variable region comprising an amino acid sequence at least 80% identical to the amino acid sequence of SEQ ID NO: 179; or
   (xcii) a heavy chain variable region comprising an amino acid sequence at least 80% identical to the amino acid sequence of SEQ ID NO: 184, and a light chain variable region comprising an amino acid sequence at least 80% identical to the amino acid sequence of SEQ ID NO: 79; or
   (xciii) a heavy chain variable region comprising an amino acid sequence at least 80% identical to the amino acid sequence of SEQ ID NO: 186, and a light chain variable region comprising an amino acid sequence at least 80% identical to the amino acid sequence of SEQ ID NO: 79; or
   (xciv) a heavy chain variable region comprising an amino acid sequence at least 80% identical to the amino acid sequence of SEQ ID NO: 188, and a light chain variable region comprising an amino acid sequence at least 80% identical to the amino acid sequence of SEQ ID NO: 79; or
   (xcv) a heavy chain variable region comprising an amino acid sequence at least 80% identical to the amino acid sequence of SEQ ID NO: 184, and a light chain variable region comprising an amino acid sequence at least 80% identical to the amino acid sequence of SEQ ID NO: 179; or
   (xcvi) a heavy chain variable region comprising an amino acid sequence at least 80% identical to the amino acid sequence of SEQ ID NO: 186, and a light chain variable region comprising an amino acid sequence at least 80% identical to the amino acid sequence of SEQ ID NO: 179; or
   (xcvii) a heavy chain variable region comprising an amino acid sequence at least 80% identical to the amino acid sequence of SEQ ID NO: 188, and a light chain variable region comprising an amino acid sequence at least 80% identical to the amino acid sequence of SEQ ID NO: 179; or
   (xcviii) a heavy chain variable region comprising an amino acid sequence at least 80% identical to the amino acid sequence of SEQ ID NO: 169, and a light chain variable region comprising an amino acid sequence at least 80% identical to the amino acid sequence of SEQ ID NO: 170.
23. The anti-LAYN antibody, or an antigen-binding fragment thereof, according to any one of the previous items, wherein the antibody or the antigen-binding fragment comprises: a heavy chain variable region comprising an amino acid sequence at least 85% identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188; and a light chain variable region comprising an amino acid sequence at least 85% identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179.
24. The anti-LAYN antibody, or an antigen-binding fragment thereof, according to any one of the previous items, wherein the antibody or the antigen-binding fragment comprises: a heavy chain variable region comprising an amino acid sequence at least 85% identical to the amino acid sequence of any one of SEQ ID NOs 7, 13, 17, 20, 22, 27, 30, 34, 78, comprising an amino acid sequence at least 85% identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179.
25. The anti-LAYN antibody, or an antigen-binding fragment thereof, according to any one of the previous items, wherein the antibody or the antigen-binding fragment comprises:
   (i) a heavy chain variable region comprising an amino acid sequence at least 85% identical to the amino acid sequence of SEQ ID NO: 7, and a light chain variable region comprising an amino acid sequence at least 85% identical to the amino acid sequence of SEQ ID NO: 8; or
   (ii) a heavy chain variable region comprising an amino acid sequence at least 85% identical to the amino acid sequence of SEQ ID NO: 13, and a light chain variable region comprising an amino acid sequence at least 85% identical to the amino acid sequence of SEQ ID NO: 14; or
   (iii) a heavy chain variable region comprising an amino acid sequence at least 85% identical to the amino acid sequence of SEQ ID NO: 17, and a light chain variable region comprising an amino acid sequence at least 85% identical to the amino acid sequence of SEQ ID NO: 8; or
   (iv) a heavy chain variable region comprising an amino acid sequence at least 85% identical to the amino acid sequence of SEQ ID NO: 20, and a light chain variable region comprising an amino acid sequence at least 85% identical to the amino acid sequence of SEQ ID NO: 14; or
   (v) a heavy chain variable region comprising an amino acid sequence at least 85% identical to the amino acid sequence of SEQ ID NO: 22, and a light chain variable region comprising an amino acid sequence at least 85% identical to the amino acid sequence of SEQ ID NO: 8; or
   (vi) a heavy chain variable region comprising an amino acid sequence at least 85% identical to the amino acid sequence of SEQ ID NO: 22, and a light chain variable region comprising an amino acid sequence at least 85% identical to the amino acid sequence of SEQ ID NO: 14; or
   (vii) a heavy chain variable region comprising an amino acid sequence at least 85% identical to the amino acid sequence of SEQ ID NO: 13, and a light chain variable region comprising an amino acid sequence at least 85% identical to the amino acid sequence of SEQ ID NO: 25; or
   (viii) a heavy chain variable region comprising an amino acid sequence at least 85% identical to the amino acid sequence of SEQ ID NO: 27, and a light chain variable region comprising an amino acid sequence at least 85% identical to the amino acid sequence of SEQ ID NO: 8; or
   (ix) a heavy chain variable region comprising an amino acid sequence at least 85% identical to the amino acid sequence of SEQ ID NO: 27, and a light chain variable region comprising an amino acid sequence at least 85% identical to the amino acid sequence of SEQ ID NO: 14; or
   (x) a heavy chain variable region comprising an amino acid sequence at least 85% identical to the amino acid sequence of SEQ ID NO: 30, and a light chain variable region comprising an amino acid sequence at least 85% identical to the amino acid sequence of SEQ ID NO: 8; or
   (xi) a heavy chain variable region comprising an amino acid sequence at least 85% identical to the amino acid sequence of SEQ ID NO: 30, and a light chain variable region comprising an amino acid sequence at least 85% identical to the amino acid sequence of SEQ ID NO: 14; or
   (xii) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 35; or
   (xiii) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 39; or
   (xiv) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 42; or
   (xv) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 25; or
   (xvi) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 45; or
   (xvii) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 48; or
   (xviii) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 51; or
   (xix) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 54; or
   (xx) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 56; or
   (xxi) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 59; or
   (xxii) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 62; or
   (xxiii) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 65; or
   (xxiv) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 68; or
   (xxv) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 71; or
   (xxvi) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 73; or
   (xxvii) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 75; or
   (xxviii) a heavy chain variable region comprising an amino acid sequence at least 85% identical to the amino acid sequence of SEQ ID NO: 78, and a light chain variable region comprising an amino acid sequence at least 85% identical to the amino acid sequence of SEQ ID NO: 79; or
   (xxix) a heavy chain variable region comprising an amino acid sequence at least 85% identical to the amino acid sequence of SEQ ID NO: 83, and a light chain variable region comprising an amino acid sequence at least 85% identical to the amino acid sequence of SEQ ID NO: 79; or
   (xxx) a heavy chain variable region comprising an amino acid sequence at least 85% identical to the amino acid sequence of SEQ ID NO: 27, and a light chain variable region comprising an amino acid sequence at least 85% identical to the amino acid sequence of SEQ ID NO: 79; or
   (xxxi) a heavy chain variable region comprising an amino acid sequence at least 85% identical to the amino acid sequence of SEQ ID NO: 86, and a light chain variable region comprising an amino acid sequence at least 85% identical to the amino acid sequence of SEQ ID NO: 79; or
   (xxxii) a heavy chain variable region comprising an amino acid sequence at least 85% identical to the amino acid sequence of SEQ ID NO: 89, and a light chain variable region comprising an amino acid sequence at least 85% identical to the amino acid sequence of SEQ ID NO: 79; or
   (xxxiii) a heavy chain variable region comprising an amino acid sequence at least 85% identical to the amino acid sequence of SEQ ID NO: 92, and a light chain variable region comprising an amino acid sequence at least 85% identical to the amino acid sequence of SEQ ID NO: 79; or
   (xxxiv) a heavy chain variable region comprising an amino acid sequence at least 85% identical to the amino acid sequence of SEQ ID NO: 95, and a light chain variable region comprising an amino acid sequence at least 85% identical to the amino acid sequence of SEQ ID NO: 79; or
   (xxxv) a heavy chain variable region comprising an amino acid sequence at least 85% identical to the amino acid sequence of SEQ ID NO: 97, and a light chain variable region comprising an amino acid sequence at least 85% identical to the amino acid sequence of SEQ ID NO: 79; or
   (xxxvi) a heavy chain variable region comprising an amino acid sequence at least 85% identical to the amino acid sequence of SEQ ID NO: 99, and a light chain variable region comprising an amino acid sequence at least 85% identical to the amino acid sequence of SEQ ID NO: 79; or
   (xxxvii) a heavy chain variable region comprising an amino acid sequence at least 85% identical to the amino acid sequence of SEQ ID NO: 101, and a light chain variable region comprising an amino acid sequence at least 85% identical to the amino acid sequence of SEQ ID NO: 79; or
   (xxxviii) a heavy chain variable region comprising an amino acid sequence at least 85% identical to the amino acid sequence of SEQ ID NO: 103, and a light chain variable region comprising an amino acid sequence at least 85% identical to the amino acid sequence of SEQ ID NO: 79; or
   (xxxix) a heavy chain variable region comprising an amino acid sequence at least 85% identical to the amino acid sequence of SEQ ID NO: 105, and a light chain variable region comprising an amino acid sequence at least 85% identical to the amino acid sequence of SEQ ID NO: 79; or
   (xl) a heavy chain variable region comprising an amino acid sequence at least 85% identical to the amino acid sequence of SEQ ID NO: 107, and a light chain variable region comprising an amino acid sequence at least 85% identical to the amino acid sequence of SEQ ID NO: 79; or
   (xli) a heavy chain variable region comprising an amino acid sequence at least 85% identical to the amino acid sequence of SEQ ID NO: 109, and a light chain variable region comprising an amino acid sequence at least 85% identical to the amino acid sequence of SEQ ID NO: 79; or
   (xlii) a heavy chain variable region comprising an amino acid sequence at least 85% identical to the amino acid sequence of SEQ ID NO: 111, and a light chain variable region comprising an amino acid sequence at least 85% identical to the amino acid sequence of SEQ ID NO: 79; or
   (xliii) a heavy chain variable region comprising an amino acid sequence at least 85% identical to the amino acid sequence of SEQ ID NO: 113, and a light chain variable region comprising an amino acid sequence at least 85% identical to the amino acid sequence of SEQ ID NO: 79; or
   (xliv) a heavy chain variable region comprising an amino acid sequence at least 85% identical to the amino acid sequence of SEQ ID NO: 115, and a light chain variable region comprising an amino acid sequence at least 85% identical to the amino acid sequence of SEQ ID NO: 79; or
   (xlv) a heavy chain variable region comprising an amino acid sequence at least 85% identical to the amino acid sequence of SEQ ID NO: 117, and a light chain variable region comprising an amino acid sequence at least 85% identical to the amino acid sequence of SEQ ID NO: 79; or
   (xlvi) a heavy chain variable region comprising an amino acid sequence at least 85% identical to the amino acid sequence of SEQ ID NO: 119, and a light chain variable region comprising an amino acid sequence at least 85% identical to the amino acid sequence of SEQ ID NO: 79; or
   (xlvii) a heavy chain variable region comprising an amino acid sequence at least 85% identical to the amino acid sequence of SEQ ID NO: 121, and a light chain variable region comprising an amino acid sequence at least 85% identical to the amino acid sequence of SEQ ID NO: 79; or
   (xlviii) a heavy chain variable region comprising an amino acid sequence at least 85% identical to the amino acid sequence of SEQ ID NO: 123, and a light chain variable region comprising an amino acid sequence at least 85% identical to the amino acid sequence of SEQ ID NO: 79; or
   (xlix) a heavy chain variable region comprising an amino acid sequence at least 85% identical to the amino acid sequence of SEQ ID NO: 126, and a light chain variable region comprising an amino acid sequence at least 85% identical to the amino acid sequence of SEQ ID NO: 79; or
   (l) a heavy chain variable region comprising an amino acid sequence at least 85% identical to the amino acid sequence of SEQ ID NO: 129, and a light chain variable region comprising an amino acid sequence at least 85% identical to the amino acid sequence of SEQ ID NO: 79; or
   (li) a heavy chain variable region comprising an amino acid sequence at least 85% identical to the amino acid sequence of SEQ ID NO: 132, and a light chain variable region comprising an amino acid sequence at least 85% identical to the amino acid sequence of SEQ ID NO: 79; or
   (lii) a heavy chain variable region comprising an amino acid sequence at least 85% identical to the amino acid sequence of SEQ ID NO: 134, and a light chain variable region comprising an amino acid sequence at least 85% identical to the amino acid sequence of SEQ ID NO: 79; or
   (liii) a heavy chain variable region comprising an amino acid sequence at least 85% identical to the amino acid sequence of SEQ ID NO: 136, and a light chain variable region comprising an amino acid sequence at least 85% identical to the amino acid sequence of SEQ ID NO: 79; or
   (liv) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 8; or
   (lv) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 14; or
   (lvi) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 138; or
   (lvii) a heavy chain variable region comprising an amino acid sequence at least 85% identical to the amino acid sequence of SEQ ID NO: 17, and a light chain variable region comprising an amino acid sequence at least 85% identical to the amino acid sequence of SEQ ID NO: 79; or
   (lviii) a heavy chain variable region comprising an amino acid sequence at least 85% identical to the amino acid sequence of SEQ ID NO: 17, and a light chain variable region comprising an amino acid sequence at least 85% identical to the amino acid sequence of SEQ ID NO: 14; or
   (lix) a heavy chain variable region comprising an amino acid sequence at least 85% identical to the amino acid sequence of SEQ ID NO: 17, and a light chain variable region comprising an amino acid sequence at least 85% identical to the amino acid sequence of SEQ ID NO: 138; or
   (lx) a heavy chain variable region comprising an amino acid sequence at least 85% identical to the amino acid sequence of SEQ ID NO: 17, and a light chain variable region comprising an amino acid sequence at least 85% identical to the amino acid sequence of SEQ ID NO: 25; or
   (lxi) a heavy chain variable region comprising an amino acid sequence at least 85% identical to the amino acid sequence of SEQ ID NO: 7, and a light chain variable region comprising an amino acid sequence at least 85% identical to the amino acid sequence of SEQ ID NO: 79; or
   (lxii) a heavy chain variable region comprising an amino acid sequence at least 85% identical to the amino acid sequence of SEQ ID NO: 7, and a light chain variable region comprising an amino acid sequence at least 85% identical to the amino acid sequence of SEQ ID NO: 14; or
   (lxiii) a heavy chain variable region comprising an amino acid sequence at least 85% identical to the amino acid sequence of SEQ ID NO: 7, and a light chain variable region comprising an amino acid sequence at least 85% identical to the amino acid sequence of SEQ ID NO: 138; or
   (lxiv) a heavy chain variable region comprising an amino acid sequence at least 85% identical to the amino acid sequence of SEQ ID NO: 7, and a light chain variable region comprising an amino acid sequence at least 85% identical to the amino acid sequence of SEQ ID NO: 25; or
   (lxv) a heavy chain variable region comprising an amino acid sequence at least 85% identical to the amino acid sequence of SEQ ID NO: 140, and a light chain variable region comprising an amino acid sequence at least 85% identical to the amino acid sequence of SEQ ID NO: 79; or
   (lxvi) a heavy chain variable region comprising an amino acid sequence at least 85% identical to the amino acid sequence of SEQ ID NO: 140, and a light chain variable region comprising an amino acid sequence at least 85% identical to the amino acid sequence of SEQ ID NO: 8; or
   (lxvii) a heavy chain variable region comprising an amino acid sequence at least 85% identical to the amino acid sequence of SEQ ID NO: 140, and a light chain variable region comprising an amino acid sequence at least 85% identical to the amino acid sequence of SEQ ID NO: 14; or
   (lxviii) a heavy chain variable region comprising an amino acid sequence at least 85% identical to the amino acid sequence of SEQ ID NO: 140, and a light chain variable region comprising an amino acid sequence at least 85% identical to the amino acid sequence of SEQ ID NO: 138; or
   (lxix) a heavy chain variable region comprising an amino acid sequence at least 85% identical to the amino acid sequence of SEQ ID NO: 140, and a light chain variable region comprising an amino acid sequence at least 85% identical to the amino acid sequence of SEQ ID NO: 25; or
   (lxx) a heavy chain variable region comprising an amino acid sequence at least 85% identical to the amino acid sequence of SEQ ID NO: 20, and a light chain variable region comprising an amino acid sequence at least 85% identical to the amino acid sequence of SEQ ID NO: 79; or
   (lxxi) a heavy chain variable region comprising an amino acid sequence at least 85% identical to the amino acid sequence of SEQ ID NO: 20, and a light chain variable region comprising an amino acid sequence at least 85% identical to the amino acid sequence of SEQ ID NO: 8; or
   (lxxii) a heavy chain variable region comprising an amino acid sequence at least 85% identical to the amino acid sequence of SEQ ID NO: 20, and a light chain variable region comprising an amino acid sequence at least 85% identical to the amino acid sequence of SEQ ID NO: 138; or
   (lxxiii) a heavy chain variable region comprising an amino acid sequence at least 85% identical to the amino acid sequence of SEQ ID NO: 20, and a light chain variable region comprising an amino acid sequence at least 85% identical to the amino acid sequence of SEQ ID NO: 25; or
   (lxxiv) a heavy chain variable region comprising an amino acid sequence at least 85% identical to the amino acid sequence of SEQ ID NO: 22, and a light chain variable region comprising an amino acid sequence at least 85% identical to the amino acid sequence of SEQ ID NO: 79; or
   (lxxv) a heavy chain variable region comprising an amino acid sequence at least 85% identical to the amino acid sequence of SEQ ID NO: 22, and a light chain variable region comprising an amino acid sequence at least 85% identical to the amino acid sequence of SEQ ID NO: 138; or
   (lxxvi) a heavy chain variable region comprising an amino acid sequence at least 85% identical to the amino acid sequence of SEQ ID NO: 22, and a light chain variable region comprising an amino acid sequence at least 85% identical to the amino acid sequence of SEQ ID NO: 25; or
   (lxxvii) a heavy chain variable region comprising an amino acid sequence at least 85% identical to the amino acid sequence of SEQ ID NO: 13, and a light chain variable region comprising an amino acid sequence at least 85% identical to the amino acid sequence of SEQ ID NO: 79; or
   (lxxviii) a heavy chain variable region comprising an amino acid sequence at least 85% identical to the amino acid sequence of SEQ ID NO: 13, and a light chain variable region comprising an amino acid sequence at least 85% identical to the amino acid sequence of SEQ ID NO: 8; or
   (lxxix) a heavy chain variable region comprising an amino acid sequence at least 85% identical to the amino acid sequence of SEQ ID NO: 13, and a light chain variable region comprising an amino acid sequence at least 85% identical to the amino acid sequence of SEQ ID NO: 138; or
   (lxxx) a heavy chain variable region comprising an amino acid sequence at least 85% identical to the amino acid sequence of SEQ ID NO: 27, and a light chain variable region comprising an amino acid sequence at least 85% identical to the amino acid sequence of SEQ ID NO: 138; or
   (lxxxi) a heavy chain variable region comprising an amino acid sequence at least 85% identical to the amino acid sequence of SEQ ID NO: 27, and a light chain variable region comprising an amino acid sequence at least 85% identical to the amino acid sequence of SEQ ID NO: 25; or
   (lxxxii) a heavy chain variable region comprising an amino acid sequence at least 85% identical to the amino acid sequence of SEQ ID NO: 30, and a light chain variable region comprising an amino acid sequence at least 85% identical to the amino acid sequence of SEQ ID NO: 79; or
   (lxxxiii) a heavy chain variable region comprising an amino acid sequence at least 85% identical to the amino acid sequence of SEQ ID NO: 30, and a light chain variable region comprising an amino acid sequence at least 85% identical to the amino acid sequence of SEQ ID NO: 138; or
   (lxxxiv) a heavy chain variable region comprising an amino acid sequence at least 85% identical to the amino acid sequence of SEQ ID NO: 30, and a light chain variable region comprising an amino acid sequence at least 85% identical to the amino acid sequence of SEQ ID NO: 25; or
   (lxxxv) a heavy chain variable region comprising an amino acid sequence at least 85% identical to the amino acid sequence of SEQ ID NO: 143, and a light chain variable region comprising an amino acid sequence at least 85% identical to the amino acid sequence of SEQ ID NO: 79; or
   (lxxxvi) a heavy chain variable region comprising an amino acid sequence at least 85% identical to the amino acid sequence of SEQ ID NO: 34, and a light chain variable region comprising an amino acid sequence at least 85% identical to the amino acid sequence of SEQ ID NO: 79; or
   (lxxxvii) a heavy chain variable region comprising an amino acid sequence at least 85% identical to the amino acid sequence of SEQ ID NO: 145, and a light chain variable region comprising an amino acid sequence at least 85% identical to the amino acid sequence of SEQ ID NO: 79; or
   (lxxxviii) a heavy chain variable region comprising an amino acid sequence at least 85% identical to the amino acid sequence of SEQ ID NO: 181, and a light chain variable region comprising an amino acid sequence at least 85% identical to the amino acid sequence of SEQ ID NO: 79; or
   (lxxxix) a heavy chain variable region comprising an amino acid sequence at least 85% identical to the amino acid sequence of SEQ ID NO: 143, and a light chain variable region comprising an amino acid sequence at least 85% identical to the amino acid sequence of SEQ ID NO: 179; or
   (xc) a heavy chain variable region comprising an amino acid sequence at least 85% identical to the amino acid sequence of SEQ ID NO: 145, and a light chain variable region comprising an amino acid sequence at least 85% identical to the amino acid sequence of SEQ ID NO: 179; or
   (xci) a heavy chain variable region comprising an amino acid sequence at least 85% identical to the amino acid sequence of SEQ ID NO: 181, and a light chain variable region comprising an amino acid sequence at least 85% identical to the amino acid sequence of SEQ ID NO: 179; or
   (xcii) a heavy chain variable region comprising an amino acid sequence at least 85% identical to the amino acid sequence of SEQ ID NO: 184, and a light chain variable region comprising an amino acid sequence at least 85% identical to the amino acid sequence of SEQ ID NO: 79; or
   (xciii) a heavy chain variable region comprising an amino acid sequence at least 85% identical to the amino acid sequence of SEQ ID NO: 186, and a light chain variable region comprising an amino acid sequence at least 85% identical to the amino acid sequence of SEQ ID NO: 79; or
   (xciv) a heavy chain variable region comprising an amino acid sequence at least 85% identical to the amino acid sequence of SEQ ID NO: 188, and a light chain variable region comprising an amino acid sequence at least 85% identical to the amino acid sequence of SEQ ID NO: 79; or
   (xcv) a heavy chain variable region comprising an amino acid sequence at least 85% identical to the amino acid sequence of SEQ ID NO: 184, and a light chain variable region comprising an amino acid sequence at least 85% identical to the amino acid sequence of SEQ ID NO: 179; or
   (xcvi) a heavy chain variable region comprising an amino acid sequence at least 85% identical to the amino acid sequence of SEQ ID NO: 186, and a light chain variable region comprising an amino acid sequence at least 85% identical to the amino acid sequence of SEQ ID NO: 179; or
   (xcvii) a heavy chain variable region comprising an amino acid sequence at least 85% identical to the amino acid sequence of SEQ ID NO: 188, and a light chain variable region comprising an amino acid sequence at least 85% identical to the amino acid sequence of SEQ ID NO: 179; or
   (xcviii) a heavy chain variable region comprising an amino acid sequence at least 85% identical to the amino acid sequence of SEQ ID NO: 169, and a light chain variable region comprising an amino acid sequence at least 85% identical to the amino acid sequence of SEQ ID NO: 170.
26. The anti-LAYN antibody, or an antigen-binding fragment thereof, according to any one of the previous items, wherein the antibody or the antigen-binding fragment comprises: a heavy chain variable region comprising an amino acid sequence at least 90% identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188; and a light chain variable region comprising an amino acid sequence at least 90% identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179.
27. The anti-LAYN antibody, or an antigen-binding fragment thereof, according to any one of the previous items, wherein the antibody or the antigen-binding fragment comprises: a heavy chain variable region comprising an amino acid sequence at least 90% identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181; and a light chain variable region comprising an amino acid sequence at least 90% identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179.
28. The anti-LAYN antibody, or an antigen-binding fragment thereof, according to any one of the previous items, wherein the antibody or the antigen-binding fragment comprises:
   (i) a heavy chain variable region comprising an amino acid sequence at least 90% identical to the amino acid sequence of SEQ ID NO: 7, and a light chain variable region comprising an amino acid sequence at least 90% identical to the amino acid sequence of SEQ ID NO: 8; or
   (ii) a heavy chain variable region comprising an amino acid sequence at least 90% identical to the amino acid sequence of SEQ ID NO: 13, and a light chain variable region comprising an amino acid sequence at least 90% identical to the amino acid sequence of SEQ ID NO: 14; or
   (iii) a heavy chain variable region comprising an amino acid sequence at least 90% identical to the amino acid sequence of SEQ ID NO: 17, and a light chain variable region comprising an amino acid sequence at least 90% identical to the amino acid sequence of SEQ ID NO: 8; or
   (iv) a heavy chain variable region comprising an amino acid sequence at least 90% identical to the amino acid sequence of SEQ ID NO: 20, and a light chain variable region comprising an amino acid sequence at least 90% identical to the amino acid sequence of SEQ ID NO: 14; or
   (v) a heavy chain variable region comprising an amino acid sequence at least 90% identical to the amino acid sequence of SEQ ID NO: 22, and a light chain variable region comprising an amino acid sequence at least 90% identical to the amino acid sequence of SEQ ID NO: 8; or
   (vi) a heavy chain variable region comprising an amino acid sequence at least 90% identical to the amino acid sequence of SEQ ID NO: 22, and a light chain variable region comprising an amino acid sequence at least 90% identical to the amino acid sequence of SEQ ID NO: 14; or
   (vii) a heavy chain variable region comprising an amino acid sequence at least 90% identical to the amino acid sequence of SEQ ID NO: 13, and a light chain variable region comprising an amino acid sequence at least 90% identical to the amino acid sequence of SEQ ID NO: 25; or
   (viii) a heavy chain variable region comprising an amino acid sequence at least 90% identical to the amino acid sequence of SEQ ID NO: 27, and a light chain variable region comprising an amino acid sequence at least 90% identical to the amino acid sequence of SEQ ID NO: 8; or
   (ix) a heavy chain variable region comprising an amino acid sequence at least 90% identical to the amino acid sequence of SEQ ID NO: 27, and a light chain variable region comprising an amino acid sequence at least 90% identical to the amino acid sequence of SEQ ID NO: 14; or
   (x) a heavy chain variable region comprising an amino acid sequence at least 90% identical to the amino acid sequence of SEQ ID NO: 30, and a light chain variable region comprising an amino acid sequence at least 90% identical to the amino acid sequence of SEQ ID NO: 8; or
   (xi) a heavy chain variable region comprising an amino acid sequence at least 90% identical to the amino acid sequence of SEQ ID NO: 30, and a light chain variable region comprising an amino acid sequence at least 90% identical to the amino acid sequence of SEQ ID NO: 14; or
   (xii) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 35; or
   (xiii) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 39; or
   (xiv) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 42; or
   (xv) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 25; or
   (xvi) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 45; or
   (xvii) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 48; or
   (xviii) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 51; or
   (xix) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 54; or
   (xx) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 56; or
   (xxi) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 59; or
   (xxii) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 62; or
   (xxiii) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 65; or
   (xxiv) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 68; or
   (xxv) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 71; or
   (xxvi) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 73; or
   (xxvii) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 75; or
   (xxviii) a heavy chain variable region comprising an amino acid sequence at least 90% identical to the amino acid sequence of SEQ ID NO: 78, and a light chain variable region comprising an amino acid sequence at least 90% identical to the amino acid sequence of SEQ ID NO: 79; or
   (xxix) a heavy chain variable region comprising an amino acid sequence at least 90% identical to the amino acid sequence of SEQ ID NO: 83, and a light chain variable region comprising an amino acid sequence at least 90% identical to the amino acid sequence of SEQ ID NO: 79; or
   (xxx) a heavy chain variable region comprising an amino acid sequence at least 90% identical to the amino acid sequence of SEQ ID NO: 27, and a light chain variable region comprising an amino acid sequence at least 90% identical to the amino acid sequence of SEQ ID NO: 79; or
   (xxxi) a heavy chain variable region comprising an amino acid sequence at least 90% identical to the amino acid sequence of SEQ ID NO: 86, and a light chain variable region comprising an amino acid sequence at least 90% identical to the amino acid sequence of SEQ ID NO: 79; or
   (xxxii) a heavy chain variable region comprising an amino acid sequence at least 90% identical to the amino acid sequence of SEQ ID NO: 89, and a light chain variable region comprising an amino acid sequence at least 90% identical to the amino acid sequence of SEQ ID NO: 79; or
   (xxxiii) a heavy chain variable region comprising an amino acid sequence at least 90% identical to the amino acid sequence of SEQ ID NO: 92, and a light chain variable region comprising an amino acid sequence at least 90% identical to the amino acid sequence of SEQ ID NO: 79; or
   (xxxiv) a heavy chain variable region comprising an amino acid sequence at least 90% identical to the amino acid sequence of SEQ ID NO: 95, and a light chain variable region comprising an amino acid sequence at least 90% identical to the amino acid sequence of SEQ ID NO: 79; or
   (xxxv) a heavy chain variable region comprising an amino acid sequence at least 90% identical to the amino acid sequence of SEQ ID NO: 97, and a light chain variable region comprising an amino acid sequence at least 90% identical to the amino acid sequence of SEQ ID NO: 79; or
   (xxxvi) a heavy chain variable region comprising an amino acid sequence at least 90% identical to the amino acid sequence of SEQ ID NO: 99, and a light chain variable region comprising an amino acid sequence at least 90% identical to the amino acid sequence of SEQ ID NO: 79; or
   (xxxvii) a heavy chain variable region comprising an amino acid sequence at least 90% identical to the amino acid sequence of SEQ ID NO: 101, and a light chain variable region comprising an amino acid sequence at least 90% identical to the amino acid sequence of SEQ ID NO: 79; or
   (xxxviii) a heavy chain variable region comprising an amino acid sequence at least 90% identical to the amino acid sequence of SEQ ID NO: 103, and a light chain variable region comprising an amino acid sequence at least 90% identical to the amino acid sequence of SEQ ID NO: 79; or
   (xxxix) a heavy chain variable region comprising an amino acid sequence at least 90% identical to the amino acid sequence of SEQ ID NO: 105, and a light chain variable region comprising an amino acid sequence at least 90% identical to the amino acid sequence of SEQ ID NO: 79; or
   (xl) a heavy chain variable region comprising an amino acid sequence at least 90% identical to the amino acid sequence of SEQ ID NO: 107, and a light chain variable region comprising an amino acid sequence at least 90% identical to the amino acid sequence of SEQ ID NO: 79; or
   (xli) a heavy chain variable region comprising an amino acid sequence at least 90% identical to the amino acid sequence of SEQ ID NO: 109, and a light chain variable region comprising an amino acid sequence at least 90% identical to the amino acid sequence of SEQ ID NO: 79; or
   (xlii) a heavy chain variable region comprising an amino acid sequence at least 90% identical to the amino acid sequence of SEQ ID NO: 111, and a light chain variable region comprising an amino acid sequence at least 90% identical to the amino acid sequence of SEQ ID NO: 79; or
   (xliii) a heavy chain variable region comprising an amino acid sequence at least 90% identical to the amino acid sequence of SEQ ID NO: 113, and a light chain variable region comprising an amino acid sequence at least 90% identical to the amino acid sequence of SEQ ID NO: 79; or
   (xliv) a heavy chain variable region comprising an amino acid sequence at least 90% identical to the amino acid sequence of SEQ ID NO: 115, and a light chain variable region comprising an amino acid sequence at least 90% identical to the amino acid sequence of SEQ ID NO: 79; or
   (xlv) a heavy chain variable region comprising an amino acid sequence at least 90% identical to the amino acid sequence of SEQ ID NO: 117, and a light chain variable region comprising an amino acid sequence at least 90% identical to the amino acid sequence of SEQ ID NO: 79; or
   (xlvi) a heavy chain variable region comprising an amino acid sequence at least 90% identical to the amino acid sequence of SEQ ID NO: 119, and a light chain variable region comprising an amino acid sequence at least 90% identical to the amino acid sequence of SEQ ID NO: 79; or
   (xlvii) a heavy chain variable region comprising an amino acid sequence at least 90% identical to the amino acid sequence of SEQ ID NO: 121, and a light chain variable region comprising an amino acid sequence at least 90% identical to the amino acid sequence of SEQ ID NO: 79; or
   (xlviii) a heavy chain variable region comprising an amino acid sequence at least 90% identical to the amino acid sequence of SEQ ID NO: 123, and a light chain variable region comprising an amino acid sequence at least 90% identical to the amino acid sequence of SEQ ID NO: 79; or
   (xlix) a heavy chain variable region comprising an amino acid sequence at least 90% identical to the amino acid sequence of SEQ ID NO: 126, and a light chain variable region comprising an amino acid sequence at least 90% identical to the amino acid sequence of SEQ ID NO: 79; or
   (l) a heavy chain variable region comprising an amino acid sequence at least 90% identical to the amino acid sequence of SEQ ID NO: 129, and a light chain variable region comprising an amino acid sequence at least 90% identical to the amino acid sequence of SEQ ID NO: 79; or
   (li) a heavy chain variable region comprising an amino acid sequence at least 90% identical to the amino acid sequence of SEQ ID NO: 132, and a light chain variable region comprising an amino acid sequence at least 90% identical to the amino acid sequence of SEQ ID NO: 79; or
   (lii) a heavy chain variable region comprising an amino acid sequence at least 90% identical to the amino acid sequence of SEQ ID NO: 134, and a light chain variable region comprising an amino acid sequence at least 90% identical to the amino acid sequence of SEQ ID NO: 79; or
   (liii) a heavy chain variable region comprising an amino acid sequence at least 90% identical to the amino acid sequence of SEQ ID NO: 136, and a light chain variable region comprising an amino acid sequence at least 90% identical to the amino acid sequence of SEQ ID NO: 79; or
   (liv) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 8; or
   (lv) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 14; or
   (lvi) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 138; or
   (lvii) a heavy chain variable region comprising an amino acid sequence at least 90% identical to the amino acid sequence of SEQ ID NO: 17, and a light chain variable region comprising an amino acid sequence at least 90% identical to the amino acid sequence of SEQ ID NO: 79; or
   (lviii) a heavy chain variable region comprising an amino acid sequence at least 90% identical to the amino acid sequence of SEQ ID NO: 17, and a light chain variable region comprising an amino acid sequence at least 90% identical to the amino acid sequence of SEQ ID NO: 14; or
   (lix) a heavy chain variable region comprising an amino acid sequence at least 90% identical to the amino acid sequence of SEQ ID NO: 17, and a light chain variable region comprising an amino acid sequence at least 90% identical to the amino acid sequence of SEQ ID NO: 138; or
   (lx) a heavy chain variable region comprising an amino acid sequence at least 90% identical to the amino acid sequence of SEQ ID NO: 17, and a light chain variable region comprising an amino acid sequence at least 90% identical to the amino acid sequence of SEQ ID NO: 25; or
   (lxi) a heavy chain variable region comprising an amino acid sequence at least 90% identical to the amino acid sequence of SEQ ID NO: 7, and a light chain variable region comprising an amino acid sequence at least 90% identical to the amino acid sequence of SEQ ID NO: 79; or
   (lxii) a heavy chain variable region comprising an amino acid sequence at least 90% identical to the amino acid sequence of SEQ ID NO: 7, and a light chain variable region comprising an amino acid sequence at least 90% identical to the amino acid sequence of SEQ ID NO: 14; or
   (lxiii) a heavy chain variable region comprising an amino acid sequence at least 90% identical to the amino acid sequence of SEQ ID NO: 7, and a light chain variable region comprising an amino acid sequence at least 90% identical to the amino acid sequence of SEQ ID NO: 138; or
   (lxiv) a heavy chain variable region comprising an amino acid sequence at least 90% identical to the amino acid sequence of SEQ ID NO: 7, and a light chain variable region comprising an amino acid sequence at least 90% identical to the amino acid sequence of SEQ ID NO: 25; or
   (lxv) a heavy chain variable region comprising an amino acid sequence at least 90% identical to the amino acid sequence of SEQ ID NO: 140, and a light chain variable region comprising an amino acid sequence at least 90% identical to the amino acid sequence of SEQ ID NO: 79; or
   (lxvi) a heavy chain variable region comprising an amino acid sequence at least 90% identical to the amino acid sequence of SEQ ID NO: 140, and a light chain variable region comprising an amino acid sequence at least 90% identical to the amino acid sequence of SEQ ID NO: 8; or
   (lxvii) a heavy chain variable region comprising an amino acid sequence at least 90% identical to the amino acid sequence of SEQ ID NO: 140, and a light chain variable region comprising an amino acid sequence at least 90% identical to the amino acid sequence of SEQ ID NO: 14; or
   (lxviii) a heavy chain variable region comprising an amino acid sequence at least 90% identical to the amino acid sequence of SEQ ID NO: 140, and a light chain variable region comprising an amino acid sequence at least 90% identical to the amino acid sequence of SEQ ID NO: 138; or
   (lxix) a heavy chain variable region comprising an amino acid sequence at least 90% identical to the amino acid sequence of SEQ ID NO: 140, and a light chain variable region comprising an amino acid sequence at least 90% identical to the amino acid sequence of SEQ ID NO: 25; or
   (lxx) a heavy chain variable region comprising an amino acid sequence at least 90% identical to the amino acid sequence of SEQ ID NO: 20, and a light chain variable region comprising an amino acid sequence at least 90% identical to the amino acid sequence of SEQ ID NO: 79; or
   (lxxi) a heavy chain variable region comprising an amino acid sequence at least 90% identical to the amino acid sequence of SEQ ID NO: 20, and a light chain variable region comprising an amino acid sequence at least 90% identical to the amino acid sequence of SEQ ID NO: 8; or
   (lxxii) a heavy chain variable region comprising an amino acid sequence at least 90% identical to the amino acid sequence of SEQ ID NO: 20, and a light chain variable region comprising an amino acid sequence at least 90% identical to the amino acid sequence of SEQ ID NO: 138; or
   (lxxiii) a heavy chain variable region comprising an amino acid sequence at least 90% identical to the amino acid sequence of SEQ ID NO: 20, and a light chain variable region comprising an amino acid sequence at least 90% identical to the amino acid sequence of SEQ ID NO: 25; or
   (lxxiv) a heavy chain variable region comprising an amino acid sequence at least 90% identical to the amino acid sequence of SEQ ID NO: 22, and a light chain variable region comprising an amino acid sequence at least 90% identical to the amino acid sequence of SEQ ID NO: 79; or
   (lxxv) a heavy chain variable region comprising an amino acid sequence at least 90% identical to the amino acid sequence of SEQ ID NO: 22, and a light chain variable region comprising an amino acid sequence at least 90% identical to the amino acid sequence of SEQ ID NO: 138; or
   (lxxvi) a heavy chain variable region comprising an amino acid sequence at least 90% identical to the amino acid sequence of SEQ ID NO: 22, and a light chain variable region comprising an amino acid sequence at least 90% identical to the amino acid sequence of SEQ ID NO: 25; or
   (lxxvii) a heavy chain variable region comprising an amino acid sequence at least 90% identical to the amino acid sequence of SEQ ID NO: 13, and a light chain variable region comprising an amino acid sequence at least 90% identical to the amino acid sequence of SEQ ID NO: 79; or
   (lxxviii) a heavy chain variable region comprising an amino acid sequence at least 90% identical to the amino acid sequence of SEQ ID NO: 13, and a light chain variable region comprising an amino acid sequence at least 90% identical to the amino acid sequence of SEQ ID NO: 8; or
   (lxxix) a heavy chain variable region comprising an amino acid sequence at least 90% identical to the amino acid sequence of SEQ ID NO: 13, and a light chain variable region comprising an amino acid sequence at least 90% identical to the amino acid sequence of SEQ ID NO: 138; or
   (lxxx) a heavy chain variable region comprising an amino acid sequence at least 90% identical to the amino acid sequence of SEQ ID NO: 27, and a light chain variable region comprising an amino acid sequence at least 90% identical to the amino acid sequence of SEQ ID NO: 138; or
   (lxxxi) a heavy chain variable region comprising an amino acid sequence at least 90% identical to the amino acid sequence of SEQ ID NO: 27, and a light chain variable region comprising an amino acid sequence at least 90% identical to the amino acid sequence of SEQ ID NO: 25; or
   (lxxxii) a heavy chain variable region comprising an amino acid sequence at least 90% identical to the amino acid sequence of SEQ ID NO: 30, and a light chain variable region comprising an amino acid sequence at least 90% identical to the amino acid sequence of SEQ ID NO: 79; or
   (lxxxiii) a heavy chain variable region comprising an amino acid sequence at least 90% identical to the amino acid sequence of SEQ ID NO: 30, and a light chain variable region comprising an amino acid sequence at least 90% identical to the amino acid sequence of SEQ ID NO: 138; or
   (lxxxiv) a heavy chain variable region comprising an amino acid sequence at least 90% identical to the amino acid sequence of SEQ ID NO: 30, and a light chain variable region comprising an amino acid sequence at least 90% identical to the amino acid sequence of SEQ ID NO: 25; or
   (lxxxv) a heavy chain variable region comprising an amino acid sequence at least 90% identical to the amino acid sequence of SEQ ID NO: 143, and a light chain variable region comprising an amino acid sequence at least 90% identical to the amino acid sequence of SEQ ID NO: 79; or
   (lxxxvi) a heavy chain variable region comprising an amino acid sequence at least 90% identical to the amino acid sequence of SEQ ID NO: 34, and a light chain variable region comprising an amino acid sequence at least 90% identical to the amino acid sequence of SEQ ID NO: 79; or
   (lxxxvii) a heavy chain variable region comprising an amino acid sequence at least 90% identical to the amino acid sequence of SEQ ID NO: 145, and a light chain variable region comprising an amino acid sequence at least 90% identical to the amino acid sequence of SEQ ID NO: 79; or
   (lxxxviii) a heavy chain variable region comprising an amino acid sequence at least 90% identical to the amino acid sequence of SEQ ID NO: 181, and a light chain variable region comprising an amino acid sequence at least 90% identical to the amino acid sequence of SEQ ID NO: 79; or
   (lxxxix) a heavy chain variable region comprising an amino acid sequence at least 90% identical to the amino acid sequence of SEQ ID NO: 143, and a light chain variable region comprising an amino acid sequence at least 90% identical to the amino acid sequence of SEQ ID NO: 179; or
   (xc) a heavy chain variable region comprising an amino acid sequence at least 90% identical to the amino acid sequence of SEQ ID NO: 145, and a light chain variable region comprising an amino acid sequence at least 90% identical to the amino acid sequence of SEQ ID NO: 179; or
   (xci) a heavy chain variable region comprising an amino acid sequence at least 90% identical to the amino acid sequence of SEQ ID NO: 181, and a light chain variable region comprising an amino acid sequence at least 90% identical to the amino acid sequence of SEQ ID NO: 179; or
   (xcii) a heavy chain variable region comprising an amino acid sequence at least 90% identical to the amino acid sequence of SEQ ID NO: 184, and a light chain variable region comprising an amino acid sequence at least 90% identical to the amino acid sequence of SEQ ID NO: 79; or
   (xciii) a heavy chain variable region comprising an amino acid sequence at least 90% identical to the amino acid sequence of SEQ ID NO: 186, and a light chain variable region comprising an amino acid sequence at least 90% identical to the amino acid sequence of SEQ ID NO: 79; or
   (xciv) a heavy chain variable region comprising an amino acid sequence at least 90% identical to the amino acid sequence of SEQ ID NO: 188, and a light chain variable region comprising an amino acid sequence at least 90% identical to the amino acid sequence of SEQ ID NO: 79; or
   (xcv) a heavy chain variable region comprising an amino acid sequence at least 90% identical to the amino acid sequence of SEQ ID NO: 184, and a light chain variable region comprising an amino acid sequence at least 90% identical to the amino acid sequence of SEQ ID NO: 179; or
   (xcvi) a heavy chain variable region comprising an amino acid sequence at least 90% identical to the amino acid sequence of SEQ ID NO: 186, and a light chain variable region comprising an amino acid sequence at least 90% identical to the amino acid sequence of SEQ ID NO: 179; or
   (xcvii) a heavy chain variable region comprising an amino acid sequence at least 90% identical to the amino acid sequence of SEQ ID NO: 188, and a light chain variable region comprising an amino acid sequence at least 90% identical to the amino acid sequence of SEQ ID NO: 179; or
   (xcviii) a heavy chain variable region comprising an amino acid sequence at least 90% identical to the amino acid sequence of SEQ ID NO: 169, and a light chain variable region comprising an amino acid sequence at least 90% identical to the amino acid sequence of SEQ ID NO: 170.
29. The anti-LAYN antibody, or an antigen-binding fragment thereof, according to any one of the previous items, wherein the antibody or the antigen-binding fragment comprises: a heavy chain variable region comprising an amino acid sequence at least 95% identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188; and a light chain variable region comprising an amino acid sequence at least 95% identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179.
30. The anti-LAYN antibody, or an antigen-binding fragment thereof, according to any one of the previous items, wherein the antibody or the antigen-binding fragment comprises: a heavy chain variable region comprising an amino acid sequence at least 95% identical to the amino acid sequence of any one of SEQ ID NOs 7, 13, 17, 20, 22, 27, 30, 34, 78, comprising an amino acid sequence at least 95% identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179.
31. The anti-LAYN antibody, or an antigen-binding fragment thereof, according to any one of the previous items, wherein the antibody or the antigen-binding fragment comprises:
   (i) a heavy chain variable region comprising an amino acid sequence at least 95% identical to the amino acid sequence of SEQ ID NO: 7, and a light chain variable region comprising an amino acid sequence at least 95% identical to the amino acid sequence of SEQ ID NO: 8; or
   (ii) a heavy chain variable region comprising an amino acid sequence at least 95% identical to the amino acid sequence of SEQ ID NO: 13, and a light chain variable region comprising an amino acid sequence at least 95% identical to the amino acid sequence of SEQ ID NO: 14; or
   (iii) a heavy chain variable region comprising an amino acid sequence at least 95% identical to the amino acid sequence of SEQ ID NO: 17, and a light chain variable region comprising an amino acid sequence at least 95% identical to the amino acid sequence of SEQ ID NO: 8; or
   (iv) a heavy chain variable region comprising an amino acid sequence at least 95% identical to the amino acid sequence of SEQ ID NO: 20, and a light chain variable region comprising an amino acid sequence at least 95% identical to the amino acid sequence of SEQ ID NO: 14; or
   (v) a heavy chain variable region comprising an amino acid sequence at least 95% identical to the amino acid sequence of SEQ ID NO: 22, and a light chain variable region comprising an amino acid sequence at least 95% identical to the amino acid sequence of SEQ ID NO: 8; or
   (vi) a heavy chain variable region comprising an amino acid sequence at least 95% identical to the amino acid sequence of SEQ ID NO: 22, and a light chain variable region comprising an amino acid sequence at least 95% identical to the amino acid sequence of SEQ ID NO: 14; or
   (vii) a heavy chain variable region comprising an amino acid sequence at least 95% identical to the amino acid sequence of SEQ ID NO: 13, and a light chain variable region comprising an amino acid sequence at least 95% identical to the amino acid sequence of SEQ ID NO: 25; or
   (viii) a heavy chain variable region comprising an amino acid sequence at least 95% identical to the amino acid sequence of SEQ ID NO: 27, and a light chain variable region comprising an amino acid sequence at least 95% identical to the amino acid sequence of SEQ ID NO: 8; or
   (ix) a heavy chain variable region comprising an amino acid sequence at least 95% identical to the amino acid sequence of SEQ ID NO: 27, and a light chain variable region comprising an amino acid sequence at least 95% identical to the amino acid sequence of SEQ ID NO: 14; or
   (x) a heavy chain variable region comprising an amino acid sequence at least 95% identical to the amino acid sequence of SEQ ID NO: 30, and a light chain variable region comprising an amino acid sequence at least 95% identical to the amino acid sequence of SEQ ID NO: 8; or
   (xi) a heavy chain variable region comprising an amino acid sequence at least 95% identical to the amino acid sequence of SEQ ID NO: 30, and a light chain variable region comprising an amino acid sequence at least 95% identical to the amino acid sequence of SEQ ID NO: 14; or
   (xii) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 35; or
   (xiii) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 39; or
   (xiv) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 42; or
   (xv) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 25; or
   (xvi) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 45; or
   (xvii) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 48; or
   (xviii) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 51; or
   (xix) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 54; or
   (xx) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 56; or
   (xxi) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 59; or
   (xxii) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 62; or
   (xxiii) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 65; or
   (xxiv) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 68; or
   (xxv) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 71; or
   (xxvi) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 73; or
   (xxvii) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 75; or
   (xxviii) a heavy chain variable region comprising an amino acid sequence at least 95% identical to the amino acid sequence of SEQ ID NO: 78, and a light chain variable region comprising an amino acid sequence at least 95% identical to the amino acid sequence of SEQ ID NO: 79; or
   (xxix) a heavy chain variable region comprising an amino acid sequence at least 95% identical to the amino acid sequence of SEQ ID NO: 83, and a light chain variable region comprising an amino acid sequence at least 95% identical to the amino acid sequence of SEQ ID NO: 79; or
   (xxx) a heavy chain variable region comprising an amino acid sequence at least 95% identical to the amino acid sequence of SEQ ID NO: 27, and a light chain variable region comprising an amino acid sequence at least 95% identical to the amino acid sequence of SEQ ID NO: 79; or
   (xxxi) a heavy chain variable region comprising an amino acid sequence at least 95% identical to the amino acid sequence of SEQ ID NO: 86, and a light chain variable region comprising an amino acid sequence at least 95% identical to the amino acid sequence of SEQ ID NO: 79; or
   (xxxii) a heavy chain variable region comprising an amino acid sequence at least 95% identical to the amino acid sequence of SEQ ID NO: 89, and a light chain variable region comprising an amino acid sequence at least 95% identical to the amino acid sequence of SEQ ID NO: 79; or
   (xxxiii) a heavy chain variable region comprising an amino acid sequence at least 95% identical to the amino acid sequence of SEQ ID NO: 92, and a light chain variable region comprising an amino acid sequence at least 95% identical to the amino acid sequence of SEQ ID NO: 79; or
   (xxxiv) a heavy chain variable region comprising an amino acid sequence at least 95% identical to the amino acid sequence of SEQ ID NO: 95, and a light chain variable region comprising an amino acid sequence at least 95% identical to the amino acid sequence of SEQ ID NO: 79; or
   (xxxv) a heavy chain variable region comprising an amino acid sequence at least 95% identical to the amino acid sequence of SEQ ID NO: 97, and a light chain variable region comprising an amino acid sequence at least 95% identical to the amino acid sequence of SEQ ID NO: 79; or
   (xxxvi) a heavy chain variable region comprising an amino acid sequence at least 95% identical to the amino acid sequence of SEQ ID NO: 99, and a light chain variable region comprising an amino acid sequence at least 95% identical to the amino acid sequence of SEQ ID NO: 79; or
   (xxxvii) a heavy chain variable region comprising an amino acid sequence at least 95% identical to the amino acid sequence of SEQ ID NO: 101, and a light chain variable region comprising an amino acid sequence at least 95% identical to the amino acid sequence of SEQ ID NO: 79; or
   (xxxviii) a heavy chain variable region comprising an amino acid sequence at least 95% identical to the amino acid sequence of SEQ ID NO: 103, and a light chain variable region comprising an amino acid sequence at least 95% identical to the amino acid sequence of SEQ ID NO: 79; or
   (xxxix) a heavy chain variable region comprising an amino acid sequence at least 95% identical to the amino acid sequence of SEQ ID NO: 105, and a light chain variable region comprising an amino acid sequence at least 95% identical to the amino acid sequence of SEQ ID NO: 79; or
   (xl) a heavy chain variable region comprising an amino acid sequence at least 95% identical to the amino acid sequence of SEQ ID NO: 107, and a light chain variable region comprising an amino acid sequence at least 95% identical to the amino acid sequence of SEQ ID NO: 79; or
   (xli) a heavy chain variable region comprising an amino acid sequence at least 95% identical to the amino acid sequence of SEQ ID NO: 109, and a light chain variable region comprising an amino acid sequence at least 95% identical to the amino acid sequence of SEQ ID NO: 79; or
   (xlii) a heavy chain variable region comprising an amino acid sequence at least 95% identical to the amino acid sequence of SEQ ID NO: 111, and a light chain variable region comprising an amino acid sequence at least 95% identical to the amino acid sequence of SEQ ID NO: 79; or
   (xliii) a heavy chain variable region comprising an amino acid sequence at least 95% identical to the amino acid sequence of SEQ ID NO: 113, and a light chain variable region comprising an amino acid sequence at least 95% identical to the amino acid sequence of SEQ ID NO: 79; or
   (xliv) a heavy chain variable region comprising an amino acid sequence at least 95% identical to the amino acid sequence of SEQ ID NO: 115, and a light chain variable region comprising an amino acid sequence at least 95% identical to the amino acid sequence of SEQ ID NO: 79; or
   (xlv) a heavy chain variable region comprising an amino acid sequence at least 95% identical to the amino acid sequence of SEQ ID NO: 117, and a light chain variable region comprising an amino acid sequence at least 95% identical to the amino acid sequence of SEQ ID NO: 79; or
   (xlvi) a heavy chain variable region comprising an amino acid sequence at least 95% identical to the amino acid sequence of SEQ ID NO: 119, and a light chain variable region comprising an amino acid sequence at least 95% identical to the amino acid sequence of SEQ ID NO: 79; or
   (xlvii) a heavy chain variable region comprising an amino acid sequence at least 95% identical to the amino acid sequence of SEQ ID NO: 121, and a light chain variable region comprising an amino acid sequence at least 95% identical to the amino acid sequence of SEQ ID NO: 79; or
   (xlviii) a heavy chain variable region comprising an amino acid sequence at least 95% identical to the amino acid sequence of SEQ ID NO: 123, and a light chain variable region comprising an amino acid sequence at least 95% identical to the amino acid sequence of SEQ ID NO: 79; or
   (xlix) a heavy chain variable region comprising an amino acid sequence at least 95% identical to the amino acid sequence of SEQ ID NO: 126, and a light chain variable region comprising an amino acid sequence at least 95% identical to the amino acid sequence of SEQ ID NO: 79; or
   (l) a heavy chain variable region comprising an amino acid sequence at least 95% identical to the amino acid sequence of SEQ ID NO: 129, and a light chain variable region comprising an amino acid sequence at least 95% identical to the amino acid sequence of SEQ ID NO: 79; or
   (li) a heavy chain variable region comprising an amino acid sequence at least 95% identical to the amino acid sequence of SEQ ID NO: 132, and a light chain variable region comprising an amino acid sequence at least 95% identical to the amino acid sequence of SEQ ID NO: 79; or
   (lii) a heavy chain variable region comprising an amino acid sequence at least 95% identical to the amino acid sequence of SEQ ID NO: 134, and a light chain variable region comprising an amino acid sequence at least 95% identical to the amino acid sequence of SEQ ID NO: 79; or
   (liii) a heavy chain variable region comprising an amino acid sequence at least 95% identical to the amino acid sequence of SEQ ID NO: 136, and a light chain variable region comprising an amino acid sequence at least 95% identical to the amino acid sequence of SEQ ID NO: 79; or
   (liv) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 8; or
   (lv) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 14; or
   (lvi) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 138; or
   (lvii) a heavy chain variable region comprising an amino acid sequence at least 95% identical to the amino acid sequence of SEQ ID NO: 17, and a light chain variable region comprising an amino acid sequence at least 95% identical to the amino acid sequence of SEQ ID NO: 79; or
   (lviii) a heavy chain variable region comprising an amino acid sequence at least 95% identical to the amino acid sequence of SEQ ID NO: 17, and a light chain variable region comprising an amino acid sequence at least 95% identical to the amino acid sequence of SEQ ID NO: 14; or
   (lix) a heavy chain variable region comprising an amino acid sequence at least 95% identical to the amino acid sequence of SEQ ID NO: 17, and a light chain variable region comprising an amino acid sequence at least 95% identical to the amino acid sequence of SEQ ID NO: 138; or
   (lx) a heavy chain variable region comprising an amino acid sequence at least 95% identical to the amino acid sequence of SEQ ID NO: 17, and a light chain variable region comprising an amino acid sequence at least 95% identical to the amino acid sequence of SEQ ID NO: 25; or
   (lxi) a heavy chain variable region comprising an amino acid sequence at least 95% identical to the amino acid sequence of SEQ ID NO: 7, and a light chain variable region comprising an amino acid sequence at least 95% identical to the amino acid sequence of SEQ ID NO: 79; or
   (lxii) a heavy chain variable region comprising an amino acid sequence at least 95% identical to the amino acid sequence of SEQ ID NO: 7, and a light chain variable region comprising an amino acid sequence at least 95% identical to the amino acid sequence of SEQ ID NO: 14; or
   (lxiii) a heavy chain variable region comprising an amino acid sequence at least 95% identical to the amino acid sequence of SEQ ID NO: 7, and a light chain variable region comprising an amino acid sequence at least 95% identical to the amino acid sequence of SEQ ID NO: 138; or
   (lxiv) a heavy chain variable region comprising an amino acid sequence at least 95% identical to the amino acid sequence of SEQ ID NO: 7, and a light chain variable region comprising an amino acid sequence at least 95% identical to the amino acid sequence of SEQ ID NO: 25; or
   (lxv) a heavy chain variable region comprising an amino acid sequence at least 95% identical to the amino acid sequence of SEQ ID NO: 140, and a light chain variable region comprising an amino acid sequence at least 95% identical to the amino acid sequence of SEQ ID NO: 79; or
   (lxvi) a heavy chain variable region comprising an amino acid sequence at least 95% identical to the amino acid sequence of SEQ ID NO: 140, and a light chain variable region comprising an amino acid sequence at least 95% identical to the amino acid sequence of SEQ ID NO: 8; or
   (lxvii) a heavy chain variable region comprising an amino acid sequence at least 95% identical to the amino acid sequence of SEQ ID NO: 140, and a light chain variable region comprising an amino acid sequence at least 95% identical to the amino acid sequence of SEQ ID NO: 14; or
   (lxviii) a heavy chain variable region comprising an amino acid sequence at least 95% identical to the amino acid sequence of SEQ ID NO: 140, and a light chain variable region comprising an amino acid sequence at least 95% identical to the amino acid sequence of SEQ ID NO: 138; or
   (lxix) a heavy chain variable region comprising an amino acid sequence at least 95% identical to the amino acid sequence of SEQ ID NO: 140, and a light chain variable region comprising an amino acid sequence at least 95% identical to the amino acid sequence of SEQ ID NO: 25; or
   (lxx) a heavy chain variable region comprising an amino acid sequence at least 95% identical to the amino acid sequence of SEQ ID NO: 20, and a light chain variable region comprising an amino acid sequence at least 95% identical to the amino acid sequence of SEQ ID NO: 79; or
   (lxxi) a heavy chain variable region comprising an amino acid sequence at least 95% identical to the amino acid sequence of SEQ ID NO: 20, and a light chain variable region comprising an amino acid sequence at least 95% identical to the amino acid sequence of SEQ ID NO: 8; or
   (lxxii) a heavy chain variable region comprising an amino acid sequence at least 95% identical to the amino acid sequence of SEQ ID NO: 20, and a light chain variable region comprising an amino acid sequence at least 95% identical to the amino acid sequence of SEQ ID NO: 138; or
   (lxxiii) a heavy chain variable region comprising an amino acid sequence at least 95% identical to the amino acid sequence of SEQ ID NO: 20, and a light chain variable region comprising an amino acid sequence at least 95% identical to the amino acid sequence of SEQ ID NO: 25; or
   (lxxiv) a heavy chain variable region comprising an amino acid sequence at least 95% identical to the amino acid sequence of SEQ ID NO: 22, and a light chain variable region comprising an amino acid sequence at least 95% identical to the amino acid sequence of SEQ ID NO: 79; or
   (lxxv) a heavy chain variable region comprising an amino acid sequence at least 95% identical to the amino acid sequence of SEQ ID NO: 22, and a light chain variable region comprising an amino acid sequence at least 95% identical to the amino acid sequence of SEQ ID NO: 138; or
   (lxxvi) a heavy chain variable region comprising an amino acid sequence at least 95% identical to the amino acid sequence of SEQ ID NO: 22, and a light chain variable region comprising an amino acid sequence at least 95% identical to the amino acid sequence of SEQ ID NO: 25; or
   (lxxvii) a heavy chain variable region comprising an amino acid sequence at least 95% identical to the amino acid sequence of SEQ ID NO: 13, and a light chain variable region comprising an amino acid sequence at least 95% identical to the amino acid sequence of SEQ ID NO: 79; or
   (lxxviii) a heavy chain variable region comprising an amino acid sequence at least 95% identical to the amino acid sequence of SEQ ID NO: 13, and a light chain variable region comprising an amino acid sequence at least 95% identical to the amino acid sequence of SEQ ID NO: 8; or
   (lxxix) a heavy chain variable region comprising an amino acid sequence at least 95% identical to the amino acid sequence of SEQ ID NO: 13, and a light chain variable region comprising an amino acid sequence at least 95% identical to the amino acid sequence of SEQ ID NO: 138; or
   (lxxx) a heavy chain variable region comprising an amino acid sequence at least 95% identical to the amino acid sequence of SEQ ID NO: 27, and a light chain variable region comprising an amino acid sequence at least 95% identical to the amino acid sequence of SEQ ID NO: 138; or
   (lxxxi) a heavy chain variable region comprising an amino acid sequence at least 95% identical to the amino acid sequence of SEQ ID NO: 27, and a light chain variable region comprising an amino acid sequence at least 95% identical to the amino acid sequence of SEQ ID NO: 25; or
   (lxxxii) a heavy chain variable region comprising an amino acid sequence at least 95% identical to the amino acid sequence of SEQ ID NO: 30, and a light chain variable region comprising an amino acid sequence at least 95% identical to the amino acid sequence of SEQ ID NO: 79; or
   (lxxxiii) a heavy chain variable region comprising an amino acid sequence at least 95% identical to the amino acid sequence of SEQ ID NO: 30, and a light chain variable region comprising an amino acid sequence at least 95% identical to the amino acid sequence of SEQ ID NO: 138; or
   (lxxxiv) a heavy chain variable region comprising an amino acid sequence at least 95% identical to the amino acid sequence of SEQ ID NO: 30, and a light chain variable region comprising an amino acid sequence at least 95% identical to the amino acid sequence of SEQ ID NO: 25; or
   (lxxxv) a heavy chain variable region comprising an amino acid sequence at least 95% identical to the amino acid sequence of SEQ ID NO: 143, and a light chain variable region comprising an amino acid sequence at least 95% identical to the amino acid sequence of SEQ ID NO: 79; or
   (lxxxvi) a heavy chain variable region comprising an amino acid sequence at least 95% identical to the amino acid sequence of SEQ ID NO: 34, and a light chain variable region comprising an amino acid sequence at least 95% identical to the amino acid sequence of SEQ ID NO: 79; or
   (lxxxvii) a heavy chain variable region comprising an amino acid sequence at least 95% identical to the amino acid sequence of SEQ ID NO: 145, and a light chain variable region comprising an amino acid sequence at least 95% identical to the amino acid sequence of SEQ ID NO: 79; or
   (lxxxviii) a heavy chain variable region comprising an amino acid sequence at least 95% identical to the amino acid sequence of SEQ ID NO: 181, and a light chain variable region comprising an amino acid sequence at least 95% identical to the amino acid sequence of SEQ ID NO: 79; or
   (lxxxix) a heavy chain variable region comprising an amino acid sequence at least 95% identical to the amino acid sequence of SEQ ID NO: 143, and a light chain variable region comprising an amino acid sequence at least 95% identical to the amino acid sequence of SEQ ID NO: 179; or
   (xc) a heavy chain variable region comprising an amino acid sequence at least 95% identical to the amino acid sequence of SEQ ID NO: 145, and a light chain variable region comprising an amino acid sequence at least 95% identical to the amino acid sequence of SEQ ID NO: 179; or
   (xci) a heavy chain variable region comprising an amino acid sequence at least 95% identical to the amino acid sequence of SEQ ID NO: 181, and a light chain variable region comprising an amino acid sequence at least 95% identical to the amino acid sequence of SEQ ID NO: 179; or
   (xcii) a heavy chain variable region comprising an amino acid sequence at least 95% identical to the amino acid sequence of SEQ ID NO: 184, and a light chain variable region comprising an amino acid sequence at least 95% identical to the amino acid sequence of SEQ ID NO: 79; or
   (xciii) a heavy chain variable region comprising an amino acid sequence at least 95% identical to the amino acid sequence of SEQ ID NO: 186, and a light chain variable region comprising an amino acid sequence at least 95% identical to the amino acid sequence of SEQ ID NO: 79; or
   (xciv) a heavy chain variable region comprising an amino acid sequence at least 95% identical to the amino acid sequence of SEQ ID NO: 188, and a light chain variable region comprising an amino acid sequence at least 95% identical to the amino acid sequence of SEQ ID NO: 79; or
   (xcv) a heavy chain variable region comprising an amino acid sequence at least 95% identical to the amino acid sequence of SEQ ID NO: 184, and a light chain variable region comprising an amino acid sequence at least 95% identical to the amino acid sequence of SEQ ID NO: 179; or
   (xcvi) a heavy chain variable region comprising an amino acid sequence at least 95% identical to the amino acid sequence of SEQ ID NO: 186, and a light chain variable region comprising an amino acid sequence at least 95% identical to the amino acid sequence of SEQ ID NO: 179; or
   (xcvii) a heavy chain variable region comprising an amino acid sequence at least 95% identical to the amino acid sequence of SEQ ID NO: 188, and a light chain variable region comprising an amino acid sequence at least 95% identical to the amino acid sequence of SEQ ID NO: 179; or
   (xcviii) a heavy chain variable region comprising an amino acid sequence at least 95% identical to the amino acid sequence of SEQ ID NO: 169, and a light chain variable region comprising an amino acid sequence at least 95% identical to the amino acid sequence of SEQ ID NO: 170.
32. The anti-LAYN antibody, or an antigen-binding fragment thereof, according to any one of the previous items, wherein the antibody or the antigen-binding fragment comprises: a heavy chain variable region comprising an amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188; and a light chain variable region comprising an amino acid sequence of any one of SEQ ID NOs 8, 14, 25, 35, 39, 42, 45, 48, 51, 54, 56, 59, 62, 65, 68, 71, 73, 75, 79, 138, 170 or 179.
33. The anti-LAYN antibody, or an antigen-binding fragment thereof, according to any one of the previous items, wherein the antibody or the antigen-binding fragment comprises: a heavy chain variable region comprising an amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181; and a light chain variable region comprising an amino acid sequence of any one of SEQ ID NOs 8, 14, 25, 35, 39, 42, 45, 48, 51, 54, 56, 59, 62, 65, 68, 71, 73, 75, 79, 138, 170 or 179.
34. The anti-LAYN antibody, or an antigen-binding fragment thereof, according to any one of the previous items, wherein the antibody or the antigen-binding fragment comprises:
   (i) a heavy chain variable region comprising the amino acid sequence of SEQ ID NO: 7, and a light chain variable region comprising the amino acid sequence of SEQ ID NO: 8; or
   (ii) a heavy chain variable region comprising the amino acid sequence of SEQ ID NO: 13, and a light chain variable region comprising the amino acid sequence of SEQ ID NO: 14; or
   (iii) a heavy chain variable region comprising the amino acid sequence of SEQ ID NO: 17, and a light chain variable region comprising the amino acid sequence of SEQ ID NO: 8; or
   (iv) a heavy chain variable region comprising the amino acid sequence of SEQ ID NO: 20, and a light chain variable region comprising the amino acid sequence of SEQ ID NO: 14; or
   (v) a heavy chain variable region comprising the amino acid sequence of SEQ ID NO: 22, and a light chain variable region comprising the amino acid sequence of SEQ ID NO: 8; or
   (vi) a heavy chain variable region comprising the amino acid sequence of SEQ ID NO: 22, and a light chain variable region comprising the amino acid sequence of SEQ ID NO: 14; or
   (vii) a heavy chain variable region comprising the amino acid sequence of SEQ ID NO: 13, and a light chain variable region comprising the amino acid sequence of SEQ ID NO: 25; or
   (viii) a heavy chain variable region comprising the amino acid sequence of SEQ ID NO: 27, and a light chain variable region comprising the amino acid sequence of SEQ ID NO: 8; or
   (ix) a heavy chain variable region comprising the amino acid sequence of SEQ ID NO: 27, and a light chain variable region comprising the amino acid sequence of SEQ ID NO: 14; or
   (x) a heavy chain variable region comprising the amino acid sequence of SEQ ID NO: 30, and a light chain variable region comprising the amino acid sequence of SEQ ID NO: 8; or
   (xi) a heavy chain variable region comprising the amino acid sequence of SEQ ID NO: 30, and a light chain variable region comprising the amino acid sequence of SEQ ID NO: 14; or
   (xii) a heavy chain variable region comprising the amino acid sequence of SEQ ID NO: 34, and a light chain variable region comprising the amino acid sequence of SEQ ID NO: 35; or
   (xiii) a heavy chain variable region comprising the amino acid sequence of SEQ ID NO: 34, and a light chain variable region comprising the amino acid sequence of SEQ ID NO: 39; or
   (xiv) a heavy chain variable region comprising the amino acid sequence of SEQ ID NO: 34, and a light chain variable region comprising the amino acid sequence of SEQ ID NO: 42; or
   (xv) a heavy chain variable region comprising the amino acid sequence of SEQ ID NO: 34, and a light chain variable region comprising the amino acid sequence of SEQ ID NO: 25; or
   (xvi) a heavy chain variable region comprising the amino acid sequence of SEQ ID NO: 34, and a light chain variable region comprising the amino acid sequence of SEQ ID NO: 45; or
   (xvii) a heavy chain variable region comprising the amino acid sequence of SEQ ID NO: 34, and a light chain variable region comprising the amino acid sequence of SEQ ID NO: 48; or
   (xviii) a heavy chain variable region comprising the amino acid sequence of SEQ ID NO: 34, and a light chain variable region comprising the amino acid sequence of SEQ ID NO: 51; or
   (xix) a heavy chain variable region comprising the amino acid sequence of SEQ ID NO: 34, and a light chain variable region comprising the amino acid sequence of SEQ ID NO: 54; or
   (xx) a heavy chain variable region comprising the amino acid sequence of SEQ ID NO: 34, and a light chain variable region comprising the amino acid sequence of SEQ ID NO: 56; or
   (xxi) a heavy chain variable region comprising the amino acid sequence of SEQ ID NO: 34, and a light chain variable region comprising the amino acid sequence of SEQ ID NO: 59; or
   (xxii) a heavy chain variable region comprising the amino acid sequence of SEQ ID NO: 34, and a light chain variable region comprising the amino acid sequence of SEQ ID NO: 62; or
   (xxiii) a heavy chain variable region comprising the amino acid sequence of SEQ ID NO: 34, and a light chain variable region comprising the amino acid sequence of SEQ ID NO: 65; or
   (xxiv) a heavy chain variable region comprising the amino acid sequence of SEQ ID NO: 34, and a light chain variable region comprising the amino acid sequence of SEQ ID NO: 68; or
   (xxv) a heavy chain variable region comprising the amino acid sequence of SEQ ID NO: 34, and a light chain variable region comprising the amino acid sequence of SEQ ID NO: 71; or
   (xxvi) a heavy chain variable region comprising the amino acid sequence of SEQ ID NO: 34, and a light chain variable region comprising the amino acid sequence of SEQ ID NO: 73; or
   (xxvii) a heavy chain variable region comprising the amino acid sequence of SEQ ID NO: 34, and a light chain variable region comprising the amino acid sequence of SEQ ID NO: 75; or
   (xxviii) a heavy chain variable region comprising the amino acid sequence of SEQ ID NO: 78, and a light chain variable region comprising the amino acid sequence of SEQ ID NO: 79; or
   (xxix) a heavy chain variable region comprising the amino acid sequence of SEQ ID NO: 83, and a light chain variable region comprising the amino acid sequence of SEQ ID NO: 79; or
   (xxx) a heavy chain variable region comprising the amino acid sequence of SEQ ID NO: 27, and a light chain variable region comprising the amino acid sequence of SEQ ID NO: 79; or
   (xxxi) a heavy chain variable region comprising the amino acid sequence of SEQ ID NO: 86, and a light chain variable region comprising the amino acid sequence of SEQ ID NO: 79; or
   (xxxii) a heavy chain variable region comprising the amino acid sequence of SEQ ID NO: 89, and a light chain variable region comprising the amino acid sequence of SEQ ID NO: 79; or
   (xxxiii) a heavy chain variable region comprising the amino acid sequence of SEQ ID NO: 92, and a light chain variable region comprising the amino acid sequence of SEQ ID NO: 79; or
   (xxxiv) a heavy chain variable region comprising the amino acid sequence of SEQ ID NO: 95, and a light chain variable region comprising the amino acid sequence of SEQ ID NO: 79; or
   (xxxv) a heavy chain variable region comprising the amino acid sequence of SEQ ID NO: 97, and a light chain variable region comprising the amino acid sequence of SEQ ID NO: 79; or
   (xxxvi) a heavy chain variable region comprising the amino acid sequence of SEQ ID NO: 99, and a light chain variable region comprising the amino acid sequence of SEQ ID NO: 79; or
   (xxxvii) a heavy chain variable region comprising the amino acid sequence of SEQ ID NO: 101, and a light chain variable region comprising the amino acid sequence of SEQ ID NO: 79; or
   (xxxviii) a heavy chain variable region comprising the amino acid sequence of SEQ ID NO: 103, and a light chain variable region comprising the amino acid sequence of SEQ ID NO: 79; or
   (xxxix) a heavy chain variable region comprising the amino acid sequence of SEQ ID NO: 105, and a light chain variable region comprising the amino acid sequence of SEQ ID NO: 79; or
   (xl) a heavy chain variable region comprising the amino acid sequence of SEQ ID NO: 107, and a light chain variable region comprising the amino acid sequence of SEQ ID NO: 79; or
   (xli) a heavy chain variable region comprising the amino acid sequence of SEQ ID NO: 109, and a light chain variable region comprising the amino acid sequence of SEQ ID NO: 79; or
   (xlii) a heavy chain variable region comprising the amino acid sequence of SEQ ID NO: 111, and a light chain variable region comprising the amino acid sequence of SEQ ID NO: 79; or
   (xliii) a heavy chain variable region comprising the amino acid sequence of SEQ ID NO: 113, and a light chain variable region comprising the amino acid sequence of SEQ ID NO: 79; or
   (xliv) a heavy chain variable region comprising the amino acid sequence of SEQ ID NO: 115, and a light chain variable region comprising the amino acid sequence of SEQ ID NO: 79; or
   (xlv) a heavy chain variable region comprising the amino acid sequence of SEQ ID NO: 117, and a light chain variable region comprising the amino acid sequence of SEQ ID NO: 79; or
   (xlvi) a heavy chain variable region comprising the amino acid sequence of SEQ ID NO: 119, and a light chain variable region comprising the amino acid sequence of SEQ ID NO: 79; or
   (xlvii) a heavy chain variable region comprising the amino acid sequence of SEQ ID NO: 121, and a light chain variable region comprising the amino acid sequence of SEQ ID NO: 79; or
   (xlviii) a heavy chain variable region comprising the amino acid sequence of SEQ ID NO: 123, and a light chain variable region comprising the amino acid sequence of SEQ ID NO: 79; or
   (xlix) a heavy chain variable region comprising the amino acid sequence of SEQ ID NO: 126, and a light chain variable region comprising the amino acid sequence of SEQ ID NO: 79; or
   (l) a heavy chain variable region comprising the amino acid sequence of SEQ ID NO: 129, and a light chain variable region comprising the amino acid sequence of SEQ ID NO: 79; or
   (li) a heavy chain variable region comprising the amino acid sequence of SEQ ID NO: 132, and a light chain variable region comprising the amino acid sequence of SEQ ID NO: 79; or
   (lii) a heavy chain variable region comprising the amino acid sequence of SEQ ID NO: 134, and a light chain variable region comprising the amino acid sequence of SEQ ID NO: 79; or
   (liii) a heavy chain variable region comprising the amino acid sequence of SEQ ID NO: 136, and a light chain variable region comprising the amino acid sequence of SEQ ID NO: 79; or
   (liv) a heavy chain variable region comprising the amino acid sequence of SEQ ID NO: 34, and a light chain variable region comprising the amino acid sequence of SEQ ID NO: 8; or
   (lv) a heavy chain variable region comprising the amino acid sequence of SEQ ID NO: 34, and a light chain variable region comprising the amino acid sequence of SEQ ID NO: 14; or
   (lvi) a heavy chain variable region comprising the amino acid sequence of SEQ ID NO: 34, and a light chain variable region comprising the amino acid sequence of SEQ ID NO: 138; or
   (lvii) a heavy chain variable region comprising the amino acid sequence of SEQ ID NO: 17, and a light chain variable region comprising the amino acid sequence of SEQ ID NO: 79; or
   (lviii) a heavy chain variable region comprising the amino acid sequence of SEQ ID NO: 17, and a light chain variable region comprising the amino acid sequence of SEQ ID NO: 14; or
   (lix) a heavy chain variable region comprising the amino acid sequence of SEQ ID NO: 17, and a light chain variable region comprising the amino acid sequence of SEQ ID NO: 138; or
   (lx) a heavy chain variable region comprising the amino acid sequence of SEQ ID NO: 17, and a light chain variable region comprising the amino acid sequence of SEQ ID NO: 25; or
   (lxi) a heavy chain variable region comprising the amino acid sequence of SEQ ID NO: 7, and a light chain variable region comprising the amino acid sequence of SEQ ID NO: 79; or
   (lxii) a heavy chain variable region comprising the amino acid sequence of SEQ ID NO: 7, and a light chain variable region comprising the amino acid sequence of SEQ ID NO: 14; or
   (lxiii) a heavy chain variable region comprising the amino acid sequence of SEQ ID NO: 7, and a light chain variable region comprising the amino acid sequence of SEQ ID NO: 138; or
   (lxiv) a heavy chain variable region comprising the amino acid sequence of SEQ ID NO: 7, and a light chain variable region comprising the amino acid sequence of SEQ ID NO: 25; or
   (lxv) a heavy chain variable region comprising the amino acid sequence of SEQ ID NO: 140, and a light chain variable region comprising the amino acid sequence of SEQ ID NO: 79; or
   (lxvi) a heavy chain variable region comprising the amino acid sequence of SEQ ID NO: 140, and a light chain variable region comprising the amino acid sequence of SEQ ID NO: 8; or
   (lxvii) a heavy chain variable region comprising the amino acid sequence of SEQ ID NO: 140, and a light chain variable region comprising the amino acid sequence of SEQ ID NO: 14; or
   (lxviii) a heavy chain variable region comprising the amino acid sequence of SEQ ID NO: 140, and a light chain variable region comprising the amino acid sequence of SEQ ID NO: 138; or
   (lxix) a heavy chain variable region comprising the amino acid sequence of SEQ ID NO: 140, and a light chain variable region comprising the amino acid sequence of SEQ ID NO: 25; or
   (lxx) a heavy chain variable region comprising the amino acid sequence of SEQ ID NO: 20, and a light chain variable region comprising the amino acid sequence of SEQ ID NO: 79; or
   (lxxi) a heavy chain variable region comprising the amino acid sequence of SEQ ID NO: 20, and a light chain variable region comprising the amino acid sequence of SEQ ID NO: 8; or
   (lxxii) a heavy chain variable region comprising the amino acid sequence of SEQ ID NO: 20, and a light chain variable region comprising the amino acid sequence of SEQ ID NO: 138; or
   (lxxiii) a heavy chain variable region comprising the amino acid sequence of SEQ ID NO: 20, and a light chain variable region comprising the amino acid sequence of SEQ ID NO: 25; or
   (lxxiv) a heavy chain variable region comprising the amino acid sequence of SEQ ID NO: 22, and a light chain variable region comprising the amino acid sequence of SEQ ID NO: 79; or
   (lxxv) a heavy chain variable region comprising the amino acid sequence of SEQ ID NO: 22, and a light chain variable region comprising the amino acid sequence of SEQ ID NO: 138; or
   (lxxvi) a heavy chain variable region comprising the amino acid sequence of SEQ ID NO: 22, and a light chain variable region comprising the amino acid sequence of SEQ ID NO: 25; or
   (lxxvii) a heavy chain variable region comprising the amino acid sequence of SEQ ID NO: 13, and a light chain variable region comprising the amino acid sequence of SEQ ID NO: 79; or
   (lxxviii) a heavy chain variable region comprising the amino acid sequence of SEQ ID NO: 13, and a light chain variable region comprising the amino acid sequence of SEQ ID NO: 8; or
   (lxxix) a heavy chain variable region comprising the amino acid sequence of SEQ ID NO: 13, and a light chain variable region comprising the amino acid sequence of SEQ ID NO: 138; or
   (lxxx) a heavy chain variable region comprising the amino acid sequence of SEQ ID NO: 27, and a light chain variable region comprising the amino acid sequence of SEQ ID NO: 138; or
   (lxxxi) a heavy chain variable region comprising the amino acid sequence of SEQ ID NO: 27, and a light chain variable region comprising the amino acid sequence of SEQ ID NO: 25; or
   (lxxxii) a heavy chain variable region comprising the amino acid sequence of SEQ ID NO: 30, and a light chain variable region comprising the amino acid sequence of SEQ ID NO: 79; or
   (lxxxiii) a heavy chain variable region comprising the amino acid sequence of SEQ ID NO: 30, and a light chain variable region comprising the amino acid sequence of SEQ ID NO: 138; or
   (lxxxiv) a heavy chain variable region comprising the amino acid sequence of SEQ ID NO: 30, and a light chain variable region comprising the amino acid sequence of SEQ ID NO: 25; or
   (lxxxv) a heavy chain variable region comprising the amino acid sequence of SEQ ID NO: 143, and a light chain variable region comprising the amino acid sequence of SEQ ID NO: 79; or
   (lxxxvi) a heavy chain variable region comprising the amino acid sequence of SEQ ID NO: 34, and a light chain variable region comprising the amino acid sequence of SEQ ID NO: 79; or
   (lxxxvii) a heavy chain variable region comprising the amino acid sequence of SEQ ID NO: 145, and a light chain variable region comprising the amino acid sequence of SEQ ID NO: 79; or
   (lxxxviii) a heavy chain variable region comprising the amino acid sequence of SEQ ID NO: 181, and a light chain variable region comprising the amino acid sequence of SEQ ID NO: 79; or
   (lxxxix) a heavy chain variable region comprising the amino acid sequence of SEQ ID NO: 143, and a light chain variable region comprising the amino acid sequence of SEQ ID NO: 179; or
   (xc) a heavy chain variable region comprising the amino acid sequence of SEQ ID NO: 145, and a light chain variable region comprising the amino acid sequence of SEQ ID NO: 179; or
   (xci) a heavy chain variable region comprising the amino acid sequence of SEQ ID NO: 181, and a light chain variable region comprising the amino acid sequence of SEQ ID NO: 179; or
   (xcii) a heavy chain variable region comprising the amino acid sequence of SEQ ID NO: 184, and a light chain variable region comprising the amino acid sequence of SEQ ID NO: 79; or
   (xciii) a heavy chain variable region comprising the amino acid sequence of SEQ ID NO: 186, and a light chain variable region comprising the amino acid sequence of SEQ ID NO: 79; or
   (xciv) a heavy chain variable region comprising the amino acid sequence of SEQ ID NO: 188, and a light chain variable region comprising the amino acid sequence of SEQ ID NO: 79; or
   (xcv) a heavy chain variable region comprising the amino acid sequence of SEQ ID NO: 184, and a light chain variable region comprising the amino acid sequence of SEQ ID NO: 179; or
   (xcvi) a heavy chain variable region comprising the amino acid sequence of SEQ ID NO: 186, and a light chain variable region comprising the amino acid sequence of SEQ ID NO: 179; or
   (xcvii) a heavy chain variable region comprising the amino acid sequence of SEQ ID NO: 188, and a light chain variable region comprising the amino acid sequence of SEQ ID NO: 179; or
   (xcviii) a heavy chain variable region comprising the amino acid sequence of SEQ ID NO: 169, and a light chain variable region comprising the amino acid sequence of SEQ ID NO: 170.
35. The anti-LAYN antibody, or an antigen-binding fragment thereof, according to any one of the previous items, wherein the antibody or the antigen-binding fragment comprises a heavy chain comprising an amino acid sequence at least 80% identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146, 182, 185, 187 or 189; and a light chain comprising an amino acid sequence at least 80% identical to the amino acid sequence of any one of SEQ ID NOs 10, 16, 26, 37, 40, 43, 46, 49, 52, 55, 57, 60, 63, 66, 69, 72, 74, 76, 81, 139 or 180.
36. The anti-LAYN antibody, or an antigen-binding fragment thereof, according to any one of the previous items, wherein the antibody or the antigen-binding fragment comprises a heavy chain comprising an amino acid sequence at least 80% identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146 or 182; and a light chain comprising an amino acid sequence at least 80% identical to the amino acid sequence of any one of SEQ ID NOS 10, 16, 26, 37, 40, 43, 46, 49, 52, 55, 57, 60, 63, 66, 69, 72, 74, 76, 81, 139 or 180.
37. The anti-LAYN antibody, or an antigen-binding fragment thereof, according to any one of the previous items, wherein the antibody or the antigen-binding fragment comprises:
   (i) a heavy chain comprising an amino acid sequence at least 80% identical to the amino acid sequence of SEQ ID NO: 9, and a light chain comprising an amino acid sequence at least 80% identical to the amino acid sequence of SEQ ID NO: 10; or
   (ii) a heavy chain comprising an amino acid sequence at least 80% identical to the amino acid sequence of SEQ ID NO: 15, and a light chain comprising an amino acid sequence at least 80% identical to the amino acid sequence of SEQ ID NO: 16; or
   (iii) a heavy chain comprising an amino acid sequence at least 80% identical to the amino acid sequence of SEQ ID NO: 18, and a light chain comprising an amino acid sequence at least 80% identical to the amino acid sequence of SEQ ID NO: 10; or
   (iv) a heavy chain comprising an amino acid sequence at least 80% identical to the amino acid sequence of SEQ ID NO: 21, and a light chain comprising an amino acid sequence at least 80% identical to the amino acid sequence of SEQ ID NO: 16; or
   (v) a heavy chain comprising an amino acid sequence at least 80% identical to the amino acid sequence of SEQ ID NO: 23, and a light chain comprising an amino acid sequence at least 80% identical to the amino acid sequence of SEQ ID NO: 10; or
   (vi) a heavy chain comprising an amino acid sequence at least 80% identical to the amino acid sequence of SEQ ID NO: 23, and a light chain comprising an amino acid sequence at least 80% identical to the amino acid sequence of SEQ ID NO: 16; or
   (vii) a heavy chain comprising an amino acid sequence at least 80% identical to the amino acid sequence of SEQ ID NO: 15, and a light chain comprising an amino acid sequence at least 80% identical to the amino acid sequence of SEQ ID NO: 26; or
   (viii) a heavy chain comprising an amino acid sequence at least 80% identical to the amino acid sequence of SEQ ID NO: 28, and a light chain comprising an amino acid sequence at least 80% identical to the amino acid sequence of SEQ ID NO: 10; or
   (ix) a heavy chain comprising an amino acid sequence at least 80% identical to the amino acid sequence of SEQ ID NO: 28, and a light chain comprising an amino acid sequence at least 80% identical to the amino acid sequence of SEQ ID NO: 16; or
   (x) a heavy chain comprising an amino acid sequence at least 80% identical to the amino acid sequence of SEQ ID NO: 31, and a light chain comprising an amino acid sequence at least 80% identical to the amino acid sequence of SEQ ID NO: 10; or
   (xi) a heavy chain comprising an amino acid sequence at least 80% identical to the amino acid sequence of SEQ ID NO: 31, and a light chain comprising an amino acid sequence at least 80% identical to the amino acid sequence of SEQ ID NO: 16; or
   (xii) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 37; or
   (xiii) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 40; or
   (xiv) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 43; or
   (xv) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 26; or
   (xvi) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 46; or
   (xvii) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 49; or
   (xviii) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 52; or
   (xix) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 55; or
   (xx) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 57; or
   (xxi) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 60; or
   (xxii) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 63; or
   (xxiii) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 66; or
   (xxiv) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 69; or
   (xxv) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 72; or
   (xxvi) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 74; or
   (xxvii) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 76; or
   (xxviii) a heavy chain comprising an amino acid sequence at least 80% identical to the amino acid sequence of SEQ ID NO: 80, and a light chain comprising an amino acid sequence at least 80% identical to the amino acid sequence of SEQ ID NO: 81; or
   (xxix) a heavy chain comprising an amino acid sequence at least 80% identical to the amino acid sequence of SEQ ID NO: 84, and a light chain comprising an amino acid sequence at least 80% identical to the amino acid sequence of SEQ ID NO: 81; or
   (xxx) a heavy chain comprising an amino acid sequence at least 80% identical to the amino acid sequence of SEQ ID NO: 28, and a light chain comprising an amino acid sequence at least 80% identical to the amino acid sequence of SEQ ID NO: 81; or
   (xxxi) a heavy chain comprising an amino acid sequence at least 80% identical to the amino acid sequence of SEQ ID NO: 87, and a light chain comprising an amino acid sequence at least 80% identical to the amino acid sequence of SEQ ID NO: 81; or
   (xxxii) a heavy chain comprising an amino acid sequence at least 80% identical to the amino acid sequence of SEQ ID NO: 90, and a light chain comprising an amino acid sequence at least 80% identical to the amino acid sequence of SEQ ID NO: 81; or
   (xxxiii) a heavy chain comprising an amino acid sequence at least 80% identical to the amino acid sequence of SEQ ID NO: 93, and a light chain comprising an amino acid sequence at least 80% identical to the amino acid sequence of SEQ ID NO: 81; or
   (xxxiv) a heavy chain comprising an amino acid sequence at least 80% identical to the amino acid sequence of SEQ ID NO: 96, and a light chain comprising an amino acid sequence at least 80% identical to the amino acid sequence of SEQ ID NO: 81; or
   (xxxv) a heavy chain comprising an amino acid sequence at least 80% identical to the amino acid sequence of SEQ ID NO: 98, and a light chain comprising an amino acid sequence at least 80% identical to the amino acid sequence of SEQ ID NO: 81; or
   (xxxvi) a heavy chain comprising an amino acid sequence at least 80% identical to the amino acid sequence of SEQ ID NO: 100, and a light chain comprising an amino acid sequence at least 80% identical to the amino acid sequence of SEQ ID NO: 81; or
   (xxxvii) a heavy chain comprising an amino acid sequence at least 80% identical to the amino acid sequence of SEQ ID NO: 102, and a light chain comprising an amino acid sequence at least 80% identical to the amino acid sequence of SEQ ID NO: 81; or
   (xxxviii) a heavy chain comprising an amino acid sequence at least 80% identical to the amino acid sequence of SEQ ID NO: 104, and a light chain comprising an amino acid sequence at least 80% identical to the amino acid sequence of SEQ ID NO: 81; or
   (xxxix) a heavy chain comprising an amino acid sequence at least 80% identical to the amino acid sequence of SEQ ID NO: 106, and a light chain comprising an amino acid sequence at least 80% identical to the amino acid sequence of SEQ ID NO: 81; or
   (xl) a heavy chain comprising an amino acid sequence at least 80% identical to the amino acid sequence of SEQ ID NO: 108, and a light chain comprising an amino acid sequence at least 80% identical to the amino acid sequence of SEQ ID NO: 81; or
   (xli) a heavy chain comprising an amino acid sequence at least 80% identical to the amino acid sequence of SEQ ID NO: 110, and a light chain comprising an amino acid sequence at least 80% identical to the amino acid sequence of SEQ ID NO: 81; or
   (xlii) a heavy chain comprising an amino acid sequence at least 80% identical to the amino acid sequence of SEQ ID NO: 112, and a light chain comprising an amino acid sequence at least 80% identical to the amino acid sequence of SEQ ID NO: 81; or
   (xliii) a heavy chain comprising an amino acid sequence at least 80% identical to the amino acid sequence of SEQ ID NO: 114, and a light chain comprising an amino acid sequence at least 80% identical to the amino acid sequence of SEQ ID NO: 81; or
   (xliv) a heavy chain comprising an amino acid sequence at least 80% identical to the amino acid sequence of SEQ ID NO: 116, and a light chain comprising an amino acid sequence at least 80% identical to the amino acid sequence of SEQ ID NO: 81; or
   (xlv) a heavy chain comprising an amino acid sequence at least 80% identical to the amino acid sequence of SEQ ID NO: 118, and a light chain comprising an amino acid sequence at least 80% identical to the amino acid sequence of SEQ ID NO: 81; or
   (xlvi) a heavy chain comprising an amino acid sequence at least 80% identical to the amino acid sequence of SEQ ID NO: 120, and a light chain comprising an amino acid sequence at least 80% identical to the amino acid sequence of SEQ ID NO: 81; or
   (xlvii) a heavy chain comprising an amino acid sequence at least 80% identical to the amino acid sequence of SEQ ID NO: 122, and a light chain comprising an amino acid sequence at least 80% identical to the amino acid sequence of SEQ ID NO: 81; or
   (xlviii) a heavy chain comprising an amino acid sequence at least 80% identical to the amino acid sequence of SEQ ID NO: 124, and a light chain comprising an amino acid sequence at least 80% identical to the amino acid sequence of SEQ ID NO: 81; or
   (xlix) a heavy chain comprising an amino acid sequence at least 80% identical to the amino acid sequence of SEQ ID NO: 127, and a light chain comprising an amino acid sequence at least 80% identical to the amino acid sequence of SEQ ID NO: 81; or
   (l) a heavy chain comprising an amino acid sequence at least 80% identical to the amino acid sequence of SEQ ID NO: 130, and a light chain comprising an amino acid sequence at least 80% identical to the amino acid sequence of SEQ ID NO: 81; or
   (li) a heavy chain comprising an amino acid sequence at least 80% identical to the amino acid sequence of SEQ ID NO: 133, and a light chain comprising an amino acid sequence at least 80% identical to the amino acid sequence of SEQ ID NO: 81; or
   (lii) a heavy chain comprising an amino acid sequence at least 80% identical to the amino acid sequence of SEQ ID NO: 135, and a light chain comprising an amino acid sequence at least 80% identical to the amino acid sequence of SEQ ID NO: 81; or
   (liii) a heavy chain comprising an amino acid sequence at least 80% identical to the amino acid sequence of SEQ ID NO: 137, and a light chain comprising an amino acid sequence at least 80% identical to the amino acid sequence of SEQ ID NO: 81; or
   (liv) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 10; or
   (lv) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 16; or
   (lvi) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 139; or
   (lvii) a heavy chain comprising an amino acid sequence at least 80% identical to the amino acid sequence of SEQ ID NO: 18, and a light chain comprising an amino acid sequence at least 80% identical to the amino acid sequence of SEQ ID NO: 81; or
   (lviii) a heavy chain comprising an amino acid sequence at least 80% identical to the amino acid sequence of SEQ ID NO: 18, and a light chain comprising an amino acid sequence at least 80% identical to the amino acid sequence of SEQ ID NO: 16; or
   (lix) a heavy chain comprising an amino acid sequence at least 80% identical to the amino acid sequence of SEQ ID NO: 18, and a light chain comprising an amino acid sequence at least 80% identical to the amino acid sequence of SEQ ID NO: 139; or
   (lx) a heavy chain comprising an amino acid sequence at least 80% identical to the amino acid sequence of SEQ ID NO: 18, and a light chain comprising an amino acid sequence at least 80% identical to the amino acid sequence of SEQ ID NO: 26; or
   (lxi) a heavy chain comprising an amino acid sequence at least 80% identical to the amino acid sequence of SEQ ID NO: 9, and a light chain comprising an amino acid sequence at least 80% identical to the amino acid sequence of SEQ ID NO: 81; or
   (lxii) a heavy chain comprising an amino acid sequence at least 80% identical to the amino acid sequence of SEQ ID NO: 9, and a light chain comprising an amino acid sequence at least 80% identical to the amino acid sequence of SEQ ID NO: 16; or
   (lxiii) a heavy chain comprising an amino acid sequence at least 80% identical to the amino acid sequence of SEQ ID NO: 9, and a light chain comprising an amino acid sequence at least 80% identical to the amino acid sequence of SEQ ID NO: 139; or
   (lxiv) a heavy chain comprising an amino acid sequence at least 80% identical to the amino acid sequence of SEQ ID NO: 9, and a light chain comprising an amino acid sequence at least 80% identical to the amino acid sequence of SEQ ID NO: 26; or
   (lxv) a heavy chain comprising an amino acid sequence at least 80% identical to the amino acid sequence of SEQ ID NO: 141, and a light chain comprising an amino acid sequence at least 80% identical to the amino acid sequence of SEQ ID NO: 81; or
   (lxvi) a heavy chain comprising an amino acid sequence at least 80% identical to the amino acid sequence of SEQ ID NO: 141, and a light chain comprising an amino acid sequence at least 80% identical to the amino acid sequence of SEQ ID NO: 10; or
   (lxvii) a heavy chain comprising an amino acid sequence at least 80% identical to the amino acid sequence of SEQ ID NO: 141, and a light chain comprising an amino acid sequence at least 80% identical to the amino acid sequence of SEQ ID NO: 16; or
   (lxviii) a heavy chain comprising an amino acid sequence at least 80% identical to the amino acid sequence of SEQ ID NO: 141, and a light chain comprising an amino acid sequence at least 80% identical to the amino acid sequence of SEQ ID NO: 139; or
   (lxix) a heavy chain comprising an amino acid sequence at least 80% identical to the amino acid sequence of SEQ ID NO: 141, and a light chain comprising an amino acid sequence at least 80% identical to the amino acid sequence of SEQ ID NO: 26; or
   (lxx) a heavy chain comprising an amino acid sequence at least 80% identical to the amino acid sequence of SEQ ID NO: 21, and a light chain comprising an amino acid sequence at least 80% identical to the amino acid sequence of SEQ ID NO: 81; or
   (lxxi) a heavy chain comprising an amino acid sequence at least 80% identical to the amino acid sequence of SEQ ID NO: 21, and a light chain comprising an amino acid sequence at least 80% identical to the amino acid sequence of SEQ ID NO: 10; or
   (lxxii) a heavy chain comprising an amino acid sequence at least 80% identical to the amino acid sequence of SEQ ID NO: 21, and a light chain comprising an amino acid sequence at least 80% identical to the amino acid sequence of SEQ ID NO: 139; or
   (lxxiii) a heavy chain comprising an amino acid sequence at least 80% identical to the amino acid sequence of SEQ ID NO: 21, and a light chain comprising an amino acid sequence at least 80% identical to the amino acid sequence of SEQ ID NO: 26; or
   (lxxiv) a heavy chain comprising an amino acid sequence at least 80% identical to the amino acid sequence of SEQ ID NO: 23, and a light chain comprising an amino acid sequence at least 80% identical to the amino acid sequence of SEQ ID NO: 81; or
   (lxxv) a heavy chain comprising an amino acid sequence at least 80% identical to the amino acid sequence of SEQ ID NO: 23, and a light chain comprising an amino acid sequence at least 80% identical to the amino acid sequence of SEQ ID NO: 139; or
   (lxxvi) a heavy chain comprising an amino acid sequence at least 80% identical to the amino acid sequence of SEQ ID NO: 23, and a light chain comprising an amino acid sequence at least 80% identical to the amino acid sequence of SEQ ID NO: 26; or
   (lxxvii) a heavy chain comprising an amino acid sequence at least 80% identical to the amino acid sequence of SEQ ID NO: 15, and a light chain comprising an amino acid sequence at least 80% identical to the amino acid sequence of SEQ ID NO: 81; or
   (lxxviii) a heavy chain comprising an amino acid sequence at least 80% identical to the amino acid sequence of SEQ ID NO: 15, and a light chain comprising an amino acid sequence at least 80% identical to the amino acid sequence of SEQ ID NO: 10; or
   (lxxix) a heavy chain comprising an amino acid sequence at least 80% identical to the amino acid sequence of SEQ ID NO: 15, and a light chain comprising an amino acid sequence at least 80% identical to the amino acid sequence of SEQ ID NO: 139; or
   (lxxx) a heavy chain comprising an amino acid sequence at least 80% identical to the amino acid sequence of SEQ ID NO: 28, and a light chain comprising an amino acid sequence at least 80% identical to the amino acid sequence of SEQ ID NO: 139; or
   (lxxxi) a heavy chain comprising an amino acid sequence at least 80% identical to the amino acid sequence of SEQ ID NO: 28, and a light chain comprising an amino acid sequence at least 80% identical to the amino acid sequence of SEQ ID NO: 26; or
   (lxxxii) a heavy chain comprising an amino acid sequence at least 80% identical to the amino acid sequence of SEQ ID NO: 31, and a light chain comprising an amino acid sequence at least 80% identical to the amino acid sequence of SEQ ID NO: 81; or
   (lxxxiii) a heavy chain comprising an amino acid sequence at least 80% identical to the amino acid sequence of SEQ ID NO: 31, and a light chain comprising an amino acid sequence at least 80% identical to the amino acid sequence of SEQ ID NO: 139; or
   (lxxxiv) a heavy chain comprising an amino acid sequence at least 80% identical to the amino acid sequence of SEQ ID NO: 31, and a light chain comprising an amino acid sequence at least 80% identical to the amino acid sequence of SEQ ID NO: 26; or
   (lxxxv) a heavy chain comprising an amino acid sequence at least 80% identical to the amino acid sequence of SEQ ID NO: 144, and a light chain comprising an amino acid sequence at least 80% identical to the amino acid sequence of SEQ ID NO: 81; or
   (lxxxvi) a heavy chain comprising an amino acid sequence at least 80% identical to the amino acid sequence of SEQ ID NO: 36, and a light chain comprising an amino acid sequence at least 80% identical to the amino acid sequence of SEQ ID NO: 81; or
   (lxxxvii) a heavy chain comprising an amino acid sequence at least 80% identical to the amino acid sequence of SEQ ID NO: 146, and a light chain comprising an amino acid sequence at least 80% identical to the amino acid sequence of SEQ ID NO: 81; or
   (lxxxviii) a heavy chain comprising an amino acid sequence at least 80% identical to the amino acid sequence of SEQ ID NO: 182, and a light chain comprising an amino acid sequence at least 80% identical to the amino acid sequence of SEQ ID NO: 81; or
   (lxxxix) a heavy chain comprising an amino acid sequence at least 80% identical to the amino acid sequence of SEQ ID NO: 144, and a light chain comprising an amino acid sequence at least 80% identical to the amino acid sequence of SEQ ID NO: 180; or
   (xc) a heavy chain comprising an amino acid sequence at least 80% identical to the amino acid sequence of SEQ ID NO: 146, and a light chain comprising an amino acid sequence at least 80% identical to the amino acid sequence of SEQ ID NO: 180; or
   (xci) a heavy chain comprising an amino acid sequence at least 80% identical to the amino acid sequence of SEQ ID NO: 182, and a light chain comprising an amino acid sequence at least 80% identical to the amino acid sequence of SEQ ID NO: 180; or
   (xcii) a heavy chain comprising an amino acid sequence at least 80% identical to the amino acid sequence of SEQ ID NO: 185, and a light chain comprising an amino acid sequence at least 80% identical to the amino acid sequence of SEQ ID NO: 81; or
   (xciii) a heavy chain comprising an amino acid sequence at least 80% identical to the amino acid sequence of SEQ ID NO: 187, and a light chain comprising an amino acid sequence at least 80% identical to the amino acid sequence of SEQ ID NO: 81; or
   (xciv) a heavy chain comprising an amino acid sequence at least 80% identical to the amino acid sequence of SEQ ID NO: 189, and a light chain comprising an amino acid sequence at least 80% identical to the amino acid sequence of SEQ ID NO: 81; or
   (xcv) a heavy chain comprising an amino acid sequence at least 80% identical to the amino acid sequence of SEQ ID NO: 185, and a light chain comprising an amino acid sequence at least 80% identical to the amino acid sequence of SEQ ID NO: 180; or
   (xcvi) a heavy chain comprising an amino acid sequence at least 80% identical to the amino acid sequence of SEQ ID NO: 187, and a light chain comprising an amino acid sequence at least 80% identical to the amino acid sequence of SEQ ID NO: 180; or
   (xcvii) a heavy chain comprising an amino acid sequence at least 80% identical to the amino acid sequence of SEQ ID NO: 189, and a light chain comprising an amino acid sequence at least 80% identical to the amino acid sequence of SEQ ID NO: 180.
38. The anti-LAYN antibody, or an antigen-binding fragment thereof, according to any one of the previous items, wherein the antibody or the antigen-binding fragment comprises a heavy chain comprising an amino acid sequence at least 85% identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146, 182, 185, 187 or 189; and a light chain comprising an amino acid sequence at least 85% identical to the amino acid sequence of any one of SEQ ID NOs 10, 16, 26, 37, 40, 43, 46, 49, 52, 55, 57, 60, 63, 66, 69, 72, 74, 76, 81, 139 or 180.
39. The anti-LAYN antibody, or an antigen-binding fragment thereof, according to any one of the previous items, wherein the antibody or the antigen-binding fragment comprises a heavy chain comprising an amino acid sequence at least 85% identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146 or 182; and a light chain comprising an amino acid sequence at least 85% identical to the amino acid sequence of any one of SEQ ID NOS 10, 16, 26, 37, 40, 43, 46, 49, 52, 55, 57, 60, 63, 66, 69, 72, 74, 76, 81, 139 or 180.
40. The anti-LAYN antibody, or an antigen-binding fragment thereof, according to any one of the previous items, wherein the antibody or the antigen-binding fragment comprises:
   (i) a heavy chain comprising an amino acid sequence at least 85% identical to the amino acid sequence of SEQ ID NO: 9, and a light chain comprising an amino acid sequence at least 85% identical to the amino acid sequence of SEQ ID NO: 10; or
   (ii) a heavy chain comprising an amino acid sequence at least 85% identical to the amino acid sequence of SEQ ID NO: 15, and a light chain comprising an amino acid sequence at least 85% identical to the amino acid sequence of SEQ ID NO: 16; or
   (iii) a heavy chain comprising an amino acid sequence at least 85% identical to the amino acid sequence of SEQ ID NO: 18, and a light chain comprising an amino acid sequence at least 85% identical to the amino acid sequence of SEQ ID NO: 10; or
   (iv) a heavy chain comprising an amino acid sequence at least 85% identical to the amino acid sequence of SEQ ID NO: 21, and a light chain comprising an amino acid sequence at least 85% identical to the amino acid sequence of SEQ ID NO: 16; or
   (v) a heavy chain comprising an amino acid sequence at least 85% identical to the amino acid sequence of SEQ ID NO: 23, and a light chain comprising an amino acid sequence at least 85% identical to the amino acid sequence of SEQ ID NO: 10; or
   (vi) a heavy chain comprising an amino acid sequence at least 85% identical to the amino acid sequence of SEQ ID NO: 23, and a light chain comprising an amino acid sequence at least 85% identical to the amino acid sequence of SEQ ID NO: 16; or
   (vii) a heavy chain comprising an amino acid sequence at least 85% identical to the amino acid sequence of SEQ ID NO: 15, and a light chain comprising an amino acid sequence at least 85% identical to the amino acid sequence of SEQ ID NO: 26; or
   (viii) a heavy chain comprising an amino acid sequence at least 85% identical to the amino acid sequence of SEQ ID NO: 28, and a light chain comprising an amino acid sequence at least 85% identical to the amino acid sequence of SEQ ID NO: 10; or
   (ix) a heavy chain comprising an amino acid sequence at least 85% identical to the amino acid sequence of SEQ ID NO: 28, and a light chain comprising an amino acid sequence at least 85% identical to the amino acid sequence of SEQ ID NO: 16; or
   (x) a heavy chain comprising an amino acid sequence at least 85% identical to the amino acid sequence of SEQ ID NO: 31, and a light chain comprising an amino acid sequence at least 85% identical to the amino acid sequence of SEQ ID NO: 10; or
   (xi) a heavy chain comprising an amino acid sequence at least 85% identical to the amino acid sequence of SEQ ID NO: 31, and a light chain comprising an amino acid sequence at least 85% identical to the amino acid sequence of SEQ ID NO: 16; or
   (xii) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 37; or
   (xiii) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 40; or
   (xiv) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 43; or
   (xv) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 26; or
   (xvi) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 46; or
   (xvii) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 49; or
   (xviii) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 52; or
   (xix) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 55; or
   (xx) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 57; or
   (xxi) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 60; or
   (xxii) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 63; or
   (xxiii) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 66; or
   (xxiv) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 69; or
   (xxv) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 72; or
   (xxvi) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 74; or
   (xxvii) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 76; or
   (xxviii) a heavy chain comprising an amino acid sequence at least 85% identical to the amino acid sequence of SEQ ID NO: 80, and a light chain comprising an amino acid sequence at least 85% identical to the amino acid sequence of SEQ ID NO: 81; or
   (xxix) a heavy chain comprising an amino acid sequence at least 85% identical to the amino acid sequence of SEQ ID NO: 84, and a light chain comprising an amino acid sequence at least 85% identical to the amino acid sequence of SEQ ID NO: 81; or
   (xxx) a heavy chain comprising an amino acid sequence at least 85% identical to the amino acid sequence of SEQ ID NO: 28, and a light chain comprising an amino acid sequence at least 85% identical to the amino acid sequence of SEQ ID NO: 81; or
   (xxxi) a heavy chain comprising an amino acid sequence at least 85% identical to the amino acid sequence of SEQ ID NO: 87, and a light chain comprising an amino acid sequence at least 85% identical to the amino acid sequence of SEQ ID NO: 81; or
   (xxxii) a heavy chain comprising an amino acid sequence at least 85% identical to the amino acid sequence of SEQ ID NO: 90, and a light chain comprising an amino acid sequence at least 85% identical to the amino acid sequence of SEQ ID NO: 81; or
   (xxxiii) a heavy chain comprising an amino acid sequence at least 85% identical to the amino acid sequence of SEQ ID NO: 93, and a light chain comprising an amino acid sequence at least 85% identical to the amino acid sequence of SEQ ID NO: 81; or
   (xxxiv) a heavy chain comprising an amino acid sequence at least 85% identical to the amino acid sequence of SEQ ID NO: 96, and a light chain comprising an amino acid sequence at least 85% identical to the amino acid sequence of SEQ ID NO: 81; or
   (xxxv) a heavy chain comprising an amino acid sequence at least 85% identical to the amino acid sequence of SEQ ID NO: 98, and a light chain comprising an amino acid sequence at least 85% identical to the amino acid sequence of SEQ ID NO: 81; or
   (xxxvi) a heavy chain comprising an amino acid sequence at least 85% identical to the amino acid sequence of SEQ ID NO: 100, and a light chain comprising an amino acid sequence at least 85% identical to the amino acid sequence of SEQ ID NO: 81; or
   (xxxvii) a heavy chain comprising an amino acid sequence at least 85% identical to the amino acid sequence of SEQ ID NO: 102, and a light chain comprising an amino acid sequence at least 85% identical to the amino acid sequence of SEQ ID NO: 81; or
   (xxxviii) a heavy chain comprising an amino acid sequence at least 85% identical to the amino acid sequence of SEQ ID NO: 104, and a light chain comprising an amino acid sequence at least 85% identical to the amino acid sequence of SEQ ID NO: 81; or (xxxix) a heavy chain comprising an amino acid sequence at least 85% identical to the amino acid sequence of SEQ ID NO: 106, and a light chain comprising an amino acid sequence at least 85% identical to the amino acid sequence of SEQ ID NO: 81; or
   (xl) a heavy chain comprising an amino acid sequence at least 85% identical to the amino acid sequence of SEQ ID NO: 108, and a light chain comprising an amino acid sequence at least 85% identical to the amino acid sequence of SEQ ID NO: 81; or
   (xli) a heavy chain comprising an amino acid sequence at least 85% identical to the amino acid sequence of SEQ ID NO: 110, and a light chain comprising an amino acid sequence at least 85% identical to the amino acid sequence of SEQ ID NO: 81; or
   (xlii) a heavy chain comprising an amino acid sequence at least 85% identical to the amino acid sequence of SEQ ID NO: 112, and a light chain comprising an amino acid sequence at least 85% identical to the amino acid sequence of SEQ ID NO: 81; or
   (xliii) a heavy chain comprising an amino acid sequence at least 85% identical to the amino acid sequence of SEQ ID NO: 114, and a light chain comprising an amino acid sequence at least 85% identical to the amino acid sequence of SEQ ID NO: 81; or
   (xliv) a heavy chain comprising an amino acid sequence at least 85% identical to the amino acid sequence of SEQ ID NO: 116, and a light chain comprising an amino acid sequence at least 85% identical to the amino acid sequence of SEQ ID NO: 81; or
   (xlv) a heavy chain comprising an amino acid sequence at least 85% identical to the amino acid sequence of SEQ ID NO: 118, and a light chain comprising an amino acid sequence at least 85% identical to the amino acid sequence of SEQ ID NO: 81; or
   (xlvi) a heavy chain comprising an amino acid sequence at least 85% identical to the amino acid sequence of SEQ ID NO: 120, and a light chain comprising an amino acid sequence at least 85% identical to the amino acid sequence of SEQ ID NO: 81; or
   (xlvii) a heavy chain comprising an amino acid sequence at least 85% identical to the amino acid sequence of SEQ ID NO: 122, and a light chain comprising an amino acid sequence at least 85% identical to the amino acid sequence of SEQ ID NO: 81; or
   (xlviii) a heavy chain comprising an amino acid sequence at least 85% identical to the amino acid sequence of SEQ ID NO: 124, and a light chain comprising an amino acid sequence at least 85% identical to the amino acid sequence of SEQ ID NO: 81; or
   (xlix) a heavy chain comprising an amino acid sequence at least 85% identical to the amino acid sequence of SEQ ID NO: 127, and a light chain comprising an amino acid sequence at least 85% identical to the amino acid sequence of SEQ ID NO: 81; or
   (l) a heavy chain comprising an amino acid sequence at least 85% identical to the amino acid sequence of SEQ ID NO: 130, and a light chain comprising an amino acid sequence at least 85% identical to the amino acid sequence of SEQ ID NO: 81; or
   (li) a heavy chain comprising an amino acid sequence at least 85% identical to the amino acid sequence of SEQ ID NO: 133, and a light chain comprising an amino acid sequence at least 85% identical to the amino acid sequence of SEQ ID NO: 81; or
   (lii) a heavy chain comprising an amino acid sequence at least 85% identical to the amino acid sequence of SEQ ID NO: 135, and a light chain comprising an amino acid sequence at least 85% identical to the amino acid sequence of SEQ ID NO: 81; or
   (liii) a heavy chain comprising an amino acid sequence at least 85% identical to the amino acid sequence of SEQ ID NO: 137, and a light chain comprising an amino acid sequence at least 85% identical to the amino acid sequence of SEQ ID NO: 81; or
   (liv) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 10; or
   (lv) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 16; or
   (lvi) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 139; or
   (lvii) a heavy chain comprising an amino acid sequence at least 85% identical to the amino acid sequence of SEQ ID NO: 18, and a light chain comprising an amino acid sequence at least 85% identical to the amino acid sequence of SEQ ID NO: 81; or
   (lviii) a heavy chain comprising an amino acid sequence at least 85% identical to the amino acid sequence of SEQ ID NO: 18, and a light chain comprising an amino acid sequence at least 85% identical to the amino acid sequence of SEQ ID NO: 16; or
   (lix) a heavy chain comprising an amino acid sequence at least 85% identical to the amino acid sequence of SEQ ID NO: 18, and a light chain comprising an amino acid sequence at least 85% identical to the amino acid sequence of SEQ ID NO: 139; or
   (lx) a heavy chain comprising an amino acid sequence at least 85% identical to the amino acid sequence of SEQ ID NO: 18, and a light chain comprising an amino acid sequence at least 85% identical to the amino acid sequence of SEQ ID NO: 26; or
   (lxi) a heavy chain comprising an amino acid sequence at least 85% identical to the amino acid sequence of SEQ ID NO: 9, and a light chain comprising an amino acid sequence at least 85% identical to the amino acid sequence of SEQ ID NO: 81; or
   (lxii) a heavy chain comprising an amino acid sequence at least 85% identical to the amino acid sequence of SEQ ID NO: 9, and a light chain comprising an amino acid sequence at least 85% identical to the amino acid sequence of SEQ ID NO: 16; or
   (lxiii) a heavy chain comprising an amino acid sequence at least 85% identical to the amino acid sequence of SEQ ID NO: 9, and a light chain comprising an amino acid sequence at least 85% identical to the amino acid sequence of SEQ ID NO: 139; or
   (lxiv) a heavy chain comprising an amino acid sequence at least 85% identical to the amino acid sequence of SEQ ID NO: 9, and a light chain comprising an amino acid sequence at least 85% identical to the amino acid sequence of SEQ ID NO: 26; or
   (lxv) a heavy chain comprising an amino acid sequence at least 85% identical to the amino acid sequence of SEQ ID NO: 141, and a light chain comprising an amino acid sequence at least 85% identical to the amino acid sequence of SEQ ID NO: 81; or
   (lxvi) a heavy chain comprising an amino acid sequence at least 85% identical to the amino acid sequence of SEQ ID NO: 141, and a light chain comprising an amino acid sequence at least 85% identical to the amino acid sequence of SEQ ID NO: 10; or
   (lxvii) a heavy chain comprising an amino acid sequence at least 85% identical to the amino acid sequence of SEQ ID NO: 141, and a light chain comprising an amino acid sequence at least 85% identical to the amino acid sequence of SEQ ID NO: 16; or
   (lxviii) a heavy chain comprising an amino acid sequence at least 85% identical to the amino acid sequence of SEQ ID NO: 141, and a light chain comprising an amino acid sequence at least 85% identical to the amino acid sequence of SEQ ID NO: 139; or
   (lxix) a heavy chain comprising an amino acid sequence at least 85% identical to the amino acid sequence of SEQ ID NO: 141, and a light chain comprising an amino acid sequence at least 85% identical to the amino acid sequence of SEQ ID NO: 26; or
   (lxx) a heavy chain comprising an amino acid sequence at least 85% identical to the amino acid sequence of SEQ ID NO: 21, and a light chain comprising an amino acid sequence at least 85% identical to the amino acid sequence of SEQ ID NO: 81; or
   (lxxi) a heavy chain comprising an amino acid sequence at least 85% identical to the amino acid sequence of SEQ ID NO: 21, and a light chain comprising an amino acid sequence at least 85% identical to the amino acid sequence of SEQ ID NO: 10; or
   (lxxii) a heavy chain comprising an amino acid sequence at least 85% identical to the amino acid sequence of SEQ ID NO: 21, and a light chain comprising an amino acid sequence at least 85% identical to the amino acid sequence of SEQ ID NO: 139; or
   (lxxiii) a heavy chain comprising an amino acid sequence at least 85% identical to the amino acid sequence of SEQ ID NO: 21, and a light chain comprising an amino acid sequence at least 85% identical to the amino acid sequence of SEQ ID NO: 26; or
   (lxxiv) a heavy chain comprising an amino acid sequence at least 85% identical to the amino acid sequence of SEQ ID NO: 23, and a light chain comprising an amino acid sequence at least 85% identical to the amino acid sequence of SEQ ID NO: 81; or
   (lxxv) a heavy chain comprising an amino acid sequence at least 85% identical to the amino acid sequence of SEQ ID NO: 23, and a light chain comprising an amino acid sequence at least 85% identical to the amino acid sequence of SEQ ID NO: 139; or
   (lxxvi) a heavy chain comprising an amino acid sequence at least 85% identical to the amino acid sequence of SEQ ID NO: 23, and a light chain comprising an amino acid sequence at least 85% identical to the amino acid sequence of SEQ ID NO: 26; or
   (lxxvii) a heavy chain comprising an amino acid sequence at least 85% identical to the amino acid sequence of SEQ ID NO: 15, and a light chain comprising an amino acid sequence at least 85% identical to the amino acid sequence of SEQ ID NO: 81; or
   (lxxviii) a heavy chain comprising an amino acid sequence at least 85% identical to the amino acid sequence of SEQ ID NO: 15, and a light chain comprising an amino acid sequence at least 85% identical to the amino acid sequence of SEQ ID NO: 10; or
   (lxxix) a heavy chain comprising an amino acid sequence at least 85% identical to the amino acid sequence of SEQ ID NO: 15, and a light chain comprising an amino acid sequence at least 85% identical to the amino acid sequence of SEQ ID NO: 139; or
   (lxxx) a heavy chain comprising an amino acid sequence at least 85% identical to the amino acid sequence of SEQ ID NO: 28, and a light chain comprising an amino acid sequence at least 85% identical to the amino acid sequence of SEQ ID NO: 139; or
   (lxxxi) a heavy chain comprising an amino acid sequence at least 85% identical to the amino acid sequence of SEQ ID NO: 28, and a light chain comprising an amino acid sequence at least 85% identical to the amino acid sequence of SEQ ID NO: 26; or
   (lxxxii) a heavy chain comprising an amino acid sequence at least 85% identical to the amino acid sequence of SEQ ID NO: 31, and a light chain comprising an amino acid sequence at least 85% identical to the amino acid sequence of SEQ ID NO: 81; or
   (lxxxiii) a heavy chain comprising an amino acid sequence at least 85% identical to the amino acid sequence of SEQ ID NO: 31, and a light chain comprising an amino acid sequence at least 85% identical to the amino acid sequence of SEQ ID NO: 139; or
   (xxxiv) a heavy chain comprising an amino acid sequence at least 85% identical to the amino acid sequence of SEQ ID NO: 31, and a light chain comprising an amino acid sequence at least 85% identical to the amino acid sequence of SEQ ID NO: 26; or
   (lxxxv) a heavy chain comprising an amino acid sequence at least 85% identical to the amino acid sequence of SEQ ID NO: 144, and a light chain comprising an amino acid sequence at least 85% identical to the amino acid sequence of SEQ ID NO: 81; or
   (lxxxvi) a heavy chain comprising an amino acid sequence at least 85% identical to the amino acid sequence of SEQ ID NO: 36, and a light chain comprising an amino acid sequence at least 85% identical to the amino acid sequence of SEQ ID NO: 81; or
   (lxxxvii) a heavy chain comprising an amino acid sequence at least 85% identical to the amino acid sequence of SEQ ID NO: 146, and a light chain comprising an amino acid sequence at least 85% identical to the amino acid sequence of SEQ ID NO: 81; or
   (lxxxviii) a heavy chain comprising an amino acid sequence at least 85% identical to the amino acid sequence of SEQ ID NO: 182, and a light chain comprising an amino acid sequence at least 85% identical to the amino acid sequence of SEQ ID NO: 81; or
   (lxxxix) a heavy chain comprising an amino acid sequence at least 85% identical to the amino acid sequence of SEQ ID NO: 144, and a light chain comprising an amino acid sequence at least 85% identical to the amino acid sequence of SEQ ID NO: 180; or
   (xc) a heavy chain comprising an amino acid sequence at least 85% identical to the amino acid sequence of SEQ ID NO: 146, and a light chain comprising an amino acid sequence at least 85% identical to the amino acid sequence of SEQ ID NO: 180; or
   (xci) a heavy chain comprising an amino acid sequence at least 85% identical to the amino acid sequence of SEQ ID NO: 182, and a light chain comprising an amino acid sequence at least 85% identical to the amino acid sequence of SEQ ID NO: 180; or
   (xcii) a heavy chain comprising an amino acid sequence at least 85% identical to the amino acid sequence of SEQ ID NO: 185, and a light chain comprising an amino acid sequence at least 85% identical to the amino acid sequence of SEQ ID NO: 81; or
   (xciii) a heavy chain comprising an amino acid sequence at least 85% identical to the amino acid sequence of SEQ ID NO: 187, and a light chain comprising an amino acid sequence at least 85% identical to the amino acid sequence of SEQ ID NO: 81; or
   (xciv) a heavy chain comprising an amino acid sequence at least 85% identical to the amino acid sequence of SEQ ID NO: 189, and a light chain comprising an amino acid sequence at least 85% identical to the amino acid sequence of SEQ ID NO: 81; or
   (xcv) a heavy chain comprising an amino acid sequence at least 85% identical to the amino acid sequence of SEQ ID NO: 185, and a light chain comprising an amino acid sequence at least 85% identical to the amino acid sequence of SEQ ID NO: 180; or
   (xcvi) a heavy chain comprising an amino acid sequence at least 85% identical to the amino acid sequence of SEQ ID NO: 187, and a light chain comprising an amino acid sequence at least 85% identical to the amino acid sequence of SEQ ID NO: 180; or
   (xcvii) a heavy chain comprising an amino acid sequence at least 85% identical to the amino acid sequence of SEQ ID NO: 189, and a light chain comprising an amino acid sequence at least 85% identical to the amino acid sequence of SEQ ID NO: 180.
41. The anti-LAYN antibody, or an antigen-binding fragment thereof, according to any one of the previous items, wherein the antibody or the antigen-binding fragment comprises a heavy chain comprising an amino acid sequence at least 90% identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146, 182, 185, 187 or 189; and a light chain comprising an amino acid sequence at least 90% identical to the amino acid sequence of any one of SEQ ID NOs 10, 16, 26, 37, 40, 43, 46, 49, 52, 55, 57, 60, 63, 66, 69, 72, 74, 76, 81, 139 or 180.
42. The anti-LAYN antibody, or an antigen-binding fragment thereof, according to any one of the previous items, wherein the antibody or the antigen-binding fragment comprises a heavy chain comprising an amino acid sequence at least 90% identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146 or 182; and a light chain comprising an amino acid sequence at least 90% identical to the amino acid sequence of any one of SEQ ID NOS 10, 16, 26, 37, 40, 43, 46, 49, 52, 55, 57, 60, 63, 66, 69, 72, 74, 76, 81, 139 or 180.
43. The anti-LAYN antibody, or an antigen-binding fragment thereof, according to any one of the previous items, wherein the antibody or the antigen-binding fragment comprises:
   (i) a heavy chain comprising an amino acid sequence at least 90% identical to the amino acid sequence of SEQ ID NO: 9, and a light chain comprising an amino acid sequence at least 90% identical to the amino acid sequence of SEQ ID NO: 10; or
   (ii) a heavy chain comprising an amino acid sequence at least 90% identical to the amino acid sequence of SEQ ID NO: 15, and a light chain comprising an amino acid sequence at least 90% identical to the amino acid sequence of SEQ ID NO: 16; or
   (iii) a heavy chain comprising an amino acid sequence at least 90% identical to the amino acid sequence of SEQ ID NO: 18, and a light chain comprising an amino acid sequence at least 90% identical to the amino acid sequence of SEQ ID NO: 10; or
   (iv) a heavy chain comprising an amino acid sequence at least 90% identical to the amino acid sequence of SEQ ID NO: 21, and a light chain comprising an amino acid sequence at least 90% identical to the amino acid sequence of SEQ ID NO: 16; or
   (v) a heavy chain comprising an amino acid sequence at least 90% identical to the amino acid sequence of SEQ ID NO: 23, and a light chain comprising an amino acid sequence at least 90% identical to the amino acid sequence of SEQ ID NO: 10; or
   (vi) a heavy chain comprising an amino acid sequence at least 90% identical to the amino acid sequence of SEQ ID NO: 23, and a light chain comprising an amino acid sequence at least 90% identical to the amino acid sequence of SEQ ID NO: 16; or
   (vii) a heavy chain comprising an amino acid sequence at least 90% identical to the amino acid sequence of SEQ ID NO: 15, and a light chain comprising an amino acid sequence at least 90% identical to the amino acid sequence of SEQ ID NO: 26; or
   (viii) a heavy chain comprising an amino acid sequence at least 90% identical to the amino acid sequence of SEQ ID NO: 28, and a light chain comprising an amino acid sequence at least 90% identical to the amino acid sequence of SEQ ID NO: 10; or
   (ix) a heavy chain comprising an amino acid sequence at least 90% identical to the amino acid sequence of SEQ ID NO: 28, and a light chain comprising an amino acid sequence at least 90% identical to the amino acid sequence of SEQ ID NO: 16; or
   (x) a heavy chain comprising an amino acid sequence at least 90% identical to the amino acid sequence of SEQ ID NO: 31, and a light chain comprising an amino acid sequence at least 90% identical to the amino acid sequence of SEQ ID NO: 10; or
   (xi) a heavy chain comprising an amino acid sequence at least 90% identical to the amino acid sequence of SEQ ID NO: 31, and a light chain comprising an amino acid sequence at least 90% identical to the amino acid sequence of SEQ ID NO: 16; or
   (xii) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 37; or
   (xiii) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 40; or
   (xiv) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 43; or
   (xv) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 26; or
   (xvi) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 46; or
   (xvii) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 49; or
   (xviii) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 52; or
   (xix) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 55; or
   (xx) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 57; or
   (xxi) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 60; or
   (xxii) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 63; or
   (xxiii) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 66; or
   (xxiv) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 69; or
   (xxv) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 72; or
   (xxvi) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 74; or
   (xxvii) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 76; or
   (xxviii) a heavy chain comprising an amino acid sequence at least 90% identical to the amino acid sequence of SEQ ID NO: 80, and a light chain comprising an amino acid sequence at least 90% identical to the amino acid sequence of SEQ ID NO: 81; or
   (xxix) a heavy chain comprising an amino acid sequence at least 90% identical to the amino acid sequence of SEQ ID NO: 84, and a light chain comprising an amino acid sequence at least 90% identical to the amino acid sequence of SEQ ID NO: 81; or
   (xxx) a heavy chain comprising an amino acid sequence at least 90% identical to the amino acid sequence of SEQ ID NO: 28, and a light chain comprising an amino acid sequence at least 90% identical to the amino acid sequence of SEQ ID NO: 81; or
   (xxxi) a heavy chain comprising an amino acid sequence at least 90% identical to the amino acid sequence of SEQ ID NO: 87, and a light chain comprising an amino acid sequence at least 90% identical to the amino acid sequence of SEQ ID NO: 81; or
   (xxxii) a heavy chain comprising an amino acid sequence at least 90% identical to the amino acid sequence of SEQ ID NO: 90, and a light chain comprising an amino acid sequence at least 90% identical to the amino acid sequence of SEQ ID NO: 81; or
   (xxxiii) a heavy chain comprising an amino acid sequence at least 90% identical to the amino acid sequence of SEQ ID NO: 93, and a light chain comprising an amino acid sequence at least 90% identical to the amino acid sequence of SEQ ID NO: 81; or
   (xxxiv) a heavy chain comprising an amino acid sequence at least 90% identical to the amino acid sequence of SEQ ID NO: 96, and a light chain comprising an amino acid sequence at least 90% identical to the amino acid sequence of SEQ ID NO: 81; or
   (xxxv) a heavy chain comprising an amino acid sequence at least 90% identical to the amino acid sequence of SEQ ID NO: 98, and a light chain comprising an amino acid sequence at least 90% identical to the amino acid sequence of SEQ ID NO: 81; or
   (xxxvi) a heavy chain comprising an amino acid sequence at least 90% identical to the amino acid sequence of SEQ ID NO: 100, and a light chain comprising an amino acid sequence at least 90% identical to the amino acid sequence of SEQ ID NO: 81; or
   (xxxvii) a heavy chain comprising an amino acid sequence at least 90% identical to the amino acid sequence of SEQ ID NO: 102, and a light chain comprising an amino acid sequence at least 90% identical to the amino acid sequence of SEQ ID NO: 81; or
   (xxxviii) a heavy chain comprising an amino acid sequence at least 90% identical to the amino acid sequence of SEQ ID NO: 104, and a light chain comprising an amino acid sequence at least 90% identical to the amino acid sequence of SEQ ID NO: 81; or
   (xxxix) a heavy chain comprising an amino acid sequence at least 90% identical to the amino acid sequence of SEQ ID NO: 106, and a light chain comprising an amino acid sequence at least 90% identical to the amino acid sequence of SEQ ID NO: 81; or
   (xl) a heavy chain comprising an amino acid sequence at least 90% identical to the amino acid sequence of SEQ ID NO: 108, and a light chain comprising an amino acid sequence at least 90% identical to the amino acid sequence of SEQ ID NO: 81; or
   (xli) a heavy chain comprising an amino acid sequence at least 90% identical to the amino acid sequence of SEQ ID NO: 110, and a light chain comprising an amino acid sequence at least 90% identical to the amino acid sequence of SEQ ID NO: 81; or
   (xlii) a heavy chain comprising an amino acid sequence at least 90% identical to the amino acid sequence of SEQ ID NO: 112, and a light chain comprising an amino acid sequence at least 90% identical to the amino acid sequence of SEQ ID NO: 81; or
   (xliii) a heavy chain comprising an amino acid sequence at least 90% identical to the amino acid sequence of SEQ ID NO: 114, and a light chain comprising an amino acid sequence at least 90% identical to the amino acid sequence of SEQ ID NO: 81; or
   (xliv) a heavy chain comprising an amino acid sequence at least 90% identical to the amino acid sequence of SEQ ID NO: 116, and a light chain comprising an amino acid sequence at least 90% identical to the amino acid sequence of SEQ ID NO: 81; or (xlv) a heavy chain comprising an amino acid sequence at least 90% identical to the amino acid sequence of SEQ ID NO: 118, and a light chain comprising an amino acid sequence at least 90% identical to the amino acid sequence of SEQ ID NO: 81; or
   (xlvi) a heavy chain comprising an amino acid sequence at least 90% identical to the amino acid sequence of SEQ ID NO: 120, and a light chain comprising an amino acid sequence at least 90% identical to the amino acid sequence of SEQ ID NO: 81; or
   (xlvii) a heavy chain comprising an amino acid sequence at least 90% identical to the amino acid sequence of SEQ ID NO: 122, and a light chain comprising an amino acid sequence at least 90% identical to the amino acid sequence of SEQ ID NO: 81; or
   (xlviii) a heavy chain comprising an amino acid sequence at least 90% identical to the amino acid sequence of SEQ ID NO: 124, and a light chain comprising an amino acid sequence at least 90% identical to the amino acid sequence of SEQ ID NO: 81; or
   (xlix) a heavy chain comprising an amino acid sequence at least 90% identical to the amino acid sequence of SEQ ID NO: 127, and a light chain comprising an amino acid sequence at least 90% identical to the amino acid sequence of SEQ ID NO: 81; or
   (l) a heavy chain comprising an amino acid sequence at least 90% identical to the amino acid sequence of SEQ ID NO: 130, and a light chain comprising an amino acid sequence at least 90% identical to the amino acid sequence of SEQ ID NO: 81; or
   (li) a heavy chain comprising an amino acid sequence at least 90% identical to the amino acid sequence of SEQ ID NO: 133, and a light chain comprising an amino acid sequence at least 90% identical to the amino acid sequence of SEQ ID NO: 81; or
   (lii) a heavy chain comprising an amino acid sequence at least 90% identical to the amino acid sequence of SEQ ID NO: 135, and a light chain comprising an amino acid sequence at least 90% identical to the amino acid sequence of SEQ ID NO: 81; or
   (liii) a heavy chain comprising an amino acid sequence at least 90% identical to the amino acid sequence of SEQ ID NO: 137, and a light chain comprising an amino acid sequence at least 90% identical to the amino acid sequence of SEQ ID NO: 81; or
   (liv) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 10; or
   (lv) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 16; or
   (lvi) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 139; or
   (lvii) a heavy chain comprising an amino acid sequence at least 90% identical to the amino acid sequence of SEQ ID NO: 18, and a light chain comprising an amino acid sequence at least 90% identical to the amino acid sequence of SEQ ID NO: 81; or
   (lviii) a heavy chain comprising an amino acid sequence at least 90% identical to the amino acid sequence of SEQ ID NO: 18, and a light chain comprising an amino acid sequence at least 90% identical to the amino acid sequence of SEQ ID NO: 16; or
   (lix) a heavy chain comprising an amino acid sequence at least 90% identical to the amino acid sequence of SEQ ID NO: 18, and a light chain comprising an amino acid sequence at least 90% identical to the amino acid sequence of SEQ ID NO: 139; or
   (lx) a heavy chain comprising an amino acid sequence at least 90% identical to the amino acid sequence of SEQ ID NO: 18, and a light chain comprising an amino acid sequence at least 90% identical to the amino acid sequence of SEQ ID NO: 26; or
   (lxi) a heavy chain comprising an amino acid sequence at least 90% identical to the amino acid sequence of SEQ ID NO: 9, and a light chain comprising an amino acid sequence at least 90% identical to the amino acid sequence of SEQ ID NO: 81; or
   (lxii) a heavy chain comprising an amino acid sequence at least 90% identical to the amino acid sequence of SEQ ID NO: 9, and a light chain comprising an amino acid sequence at least 90% identical to the amino acid sequence of SEQ ID NO: 16; or
   (lxiii) a heavy chain comprising an amino acid sequence at least 90% identical to the amino acid sequence of SEQ ID NO: 9, and a light chain comprising an amino acid sequence at least 90% identical to the amino acid sequence of SEQ ID NO: 139; or
   (lxiv) a heavy chain comprising an amino acid sequence at least 90% identical to the amino acid sequence of SEQ ID NO: 9, and a light chain comprising an amino acid sequence at least 90% identical to the amino acid sequence of SEQ ID NO: 26; or
   (lxv) a heavy chain comprising an amino acid sequence at least 90% identical to the amino acid sequence of SEQ ID NO: 141, and a light chain comprising an amino acid sequence at least 90% identical to the amino acid sequence of SEQ ID NO: 81; or
   (lxvi) a heavy chain comprising an amino acid sequence at least 90% identical to the amino acid sequence of SEQ ID NO: 141, and a light chain comprising an amino acid sequence at least 90% identical to the amino acid sequence of SEQ ID NO: 10; or
   (lxvii) a heavy chain comprising an amino acid sequence at least 90% identical to the amino acid sequence of SEQ ID NO: 141, and a light chain comprising an amino acid sequence at least 90% identical to the amino acid sequence of SEQ ID NO: 16; or
   (lxviii) a heavy chain comprising an amino acid sequence at least 90% identical to the amino acid sequence of SEQ ID NO: 141, and a light chain comprising an amino acid sequence at least 90% identical to the amino acid sequence of SEQ ID NO: 139; or
   (lxix) a heavy chain comprising an amino acid sequence at least 90% identical to the amino acid sequence of SEQ ID NO: 141, and a light chain comprising an amino acid sequence at least 90% identical to the amino acid sequence of SEQ ID NO: 26; or
   (lxx) a heavy chain comprising an amino acid sequence at least 90% identical to the amino acid sequence of SEQ ID NO: 21, and a light chain comprising an amino acid sequence at least 90% identical to the amino acid sequence of SEQ ID NO: 81; or
   (lxxi) a heavy chain comprising an amino acid sequence at least 90% identical to the amino acid sequence of SEQ ID NO: 21, and a light chain comprising an amino acid sequence at least 90% identical to the amino acid sequence of SEQ ID NO: 10; or
   (lxxii) a heavy chain comprising an amino acid sequence at least 90% identical to the amino acid sequence of SEQ ID NO: 21, and a light chain comprising an amino acid sequence at least 90% identical to the amino acid sequence of SEQ ID NO: 139; or
   (lxxiii) a heavy chain comprising an amino acid sequence at least 90% identical to the amino acid sequence of SEQ ID NO: 21, and a light chain comprising an amino acid sequence at least 90% identical to the amino acid sequence of SEQ ID NO: 26; or
   (lxxiv) a heavy chain comprising an amino acid sequence at least 90% identical to the amino acid sequence of SEQ ID NO: 23, and a light chain comprising an amino acid sequence at least 90% identical to the amino acid sequence of SEQ ID NO: 81; or
   (lxxv) a heavy chain comprising an amino acid sequence at least 90% identical to the amino acid sequence of SEQ ID NO: 23, and a light chain comprising an amino acid sequence at least 90% identical to the amino acid sequence of SEQ ID NO: 139; or
   (lxxvi) a heavy chain comprising an amino acid sequence at least 90% identical to the amino acid sequence of SEQ ID NO: 23, and a light chain comprising an amino acid sequence at least 90% identical to the amino acid sequence of SEQ ID NO: 26; or
   (lxxvii) a heavy chain comprising an amino acid sequence at least 90% identical to the amino acid sequence of SEQ ID NO: 15, and a light chain comprising an amino acid sequence at least 90% identical to the amino acid sequence of SEQ ID NO: 81; or
   (lxxviii) a heavy chain comprising an amino acid sequence at least 90% identical to the amino acid sequence of SEQ ID NO: 15, and a light chain comprising an amino acid sequence at least 90% identical to the amino acid sequence of SEQ ID NO: 10; or
   (lxxix) a heavy chain comprising an amino acid sequence at least 90% identical to the amino acid sequence of SEQ ID NO: 15, and a light chain comprising an amino acid sequence at least 90% identical to the amino acid sequence of SEQ ID NO: 139; or
   (lxxx) a heavy chain comprising an amino acid sequence at least 90% identical to the amino acid sequence of SEQ ID NO: 28, and a light chain comprising an amino acid sequence at least 90% identical to the amino acid sequence of SEQ ID NO: 139; or
   (lxxxi) a heavy chain comprising an amino acid sequence at least 90% identical to the amino acid sequence of SEQ ID NO: 28, and a light chain comprising an amino acid sequence at least 90% identical to the amino acid sequence of SEQ ID NO: 26; or
   (lxxxii) a heavy chain comprising an amino acid sequence at least 90% identical to the amino acid sequence of SEQ ID NO: 31, and a light chain comprising an amino acid sequence at least 90% identical to the amino acid sequence of SEQ ID NO: 81; or
   (lxxxiii) a heavy chain comprising an amino acid sequence at least 90% identical to the amino acid sequence of SEQ ID NO: 31, and a light chain comprising an amino acid sequence at least 90% identical to the amino acid sequence of SEQ ID NO: 139; or
   (lxxxiv) a heavy chain comprising an amino acid sequence at least 90% identical to the amino acid sequence of SEQ ID NO: 31, and a light chain comprising an amino acid sequence at least 90% identical to the amino acid sequence of SEQ ID NO: 26; or
   (lxxxv) a heavy chain comprising an amino acid sequence at least 90% identical to the amino acid sequence of SEQ ID NO: 144, and a light chain comprising an amino acid sequence at least 90% identical to the amino acid sequence of SEQ ID NO: 81; or
   (lxxxvi) a heavy chain comprising an amino acid sequence at least 90% identical to the amino acid sequence of SEQ ID NO: 36, and a light chain comprising an amino acid sequence at least 90% identical to the amino acid sequence of SEQ ID NO: 81; or
   (lxxxvii) a heavy chain comprising an amino acid sequence at least 90% identical to the amino acid sequence of SEQ ID NO: 146, and a light chain comprising an amino acid sequence at least 90% identical to the amino acid sequence of SEQ ID NO: 81; or
   (lxxxviii) a heavy chain comprising an amino acid sequence at least 90% identical to the amino acid sequence of SEQ ID NO: 182, and a light chain comprising an amino acid sequence at least 90% identical to the amino acid sequence of SEQ ID NO: 81; or
   (lxxxix) a heavy chain comprising an amino acid sequence at least 90% identical to the amino acid sequence of SEQ ID NO: 144, and a light chain comprising an amino acid sequence at least 90% identical to the amino acid sequence of SEQ ID NO: 180; or
   (xc) a heavy chain comprising an amino acid sequence at least 90% identical to the amino acid sequence of SEQ ID NO: 146, and a light chain comprising an amino acid sequence at least 90% identical to the amino acid sequence of SEQ ID NO: 180; or
   (xci) a heavy chain comprising an amino acid sequence at least 90% identical to the amino acid sequence of SEQ ID NO: 182, and a light chain comprising an amino acid sequence at least 90% identical to the amino acid sequence of SEQ ID NO: 180; or
   (xcii) a heavy chain comprising an amino acid sequence at least 90% identical to the amino acid sequence of SEQ ID NO: 185, and a light chain comprising an amino acid sequence at least 90% identical to the amino acid sequence of SEQ ID NO: 81; or
   (xciii) a heavy chain comprising an amino acid sequence at least 90% identical to the amino acid sequence of SEQ ID NO: 187, and a light chain comprising an amino acid sequence at least 90% identical to the amino acid sequence of SEQ ID NO: 81; or
   (xciv) a heavy chain comprising an amino acid sequence at least 90% identical to the amino acid sequence of SEQ ID NO: 189, and a light chain comprising an amino acid sequence at least 90% identical to the amino acid sequence of SEQ ID NO: 81; or
   (xcv) a heavy chain comprising an amino acid sequence at least 90% identical to the amino acid sequence of SEQ ID NO: 185, and a light chain comprising an amino acid sequence at least 90% identical to the amino acid sequence of SEQ ID NO: 180; or
   (xcvi) a heavy chain comprising an amino acid sequence at least 90% identical to the amino acid sequence of SEQ ID NO: 187, and a light chain comprising an amino acid sequence at least 90% identical to the amino acid sequence of SEQ ID NO: 180; or
   (xcvii) a heavy chain comprising an amino acid sequence at least 90% identical to the amino acid sequence of SEQ ID NO: 189, and a light chain comprising an amino acid sequence at least 90% identical to the amino acid sequence of SEQ ID NO: 180.
44. The anti-LAYN antibody, or an antigen-binding fragment thereof, according to any one of the previous items, wherein the antibody or the antigen-binding fragment comprises a heavy chain comprising an amino acid sequence at least 95% identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146, 182, 185, 187 or 189; and a light chain comprising an amino acid sequence at least 95% identical to the amino acid sequence of any one of SEQ ID NOs 10, 16, 26, 37, 40, 43, 46, 49, 52, 55, 57, 60, 63, 66, 69, 72, 74, 76, 81, 139 or 180.
45. The anti-LAYN antibody, or an antigen-binding fragment thereof, according to any one of the previous items, wherein the antibody or the antigen-binding fragment comprises a heavy chain comprising an amino acid sequence at least 95% identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146 or 182; and a light chain comprising an amino acid sequence at least 95% identical to the amino acid sequence of any one of SEQ ID NOS 10, 16, 26, 37, 40, 43, 46, 49, 52, 55, 57, 60, 63, 66, 69, 72, 74, 76, 81, 139 or 180.
46. The anti-LAYN antibody, or an antigen-binding fragment thereof, according to any one of the previous items, wherein the antibody or the antigen-binding fragment comprises:
   (i) a heavy chain comprising an amino acid sequence at least 95% identical to the amino acid sequence of SEQ ID NO: 9, and a light chain comprising an amino acid sequence at least 95% identical to the amino acid sequence of SEQ ID NO: 10; or
   (ii) a heavy chain comprising an amino acid sequence at least 95% identical to the amino acid sequence of SEQ ID NO: 15, and a light chain comprising an amino acid sequence at least 95% identical to the amino acid sequence of SEQ ID NO: 16; or
   (iii) a heavy chain comprising an amino acid sequence at least 95% identical to the amino acid sequence of SEQ ID NO: 18, and a light chain comprising an amino acid sequence at least 95% identical to the amino acid sequence of SEQ ID NO: 10; or
   (iv) a heavy chain comprising an amino acid sequence at least 95% identical to the amino acid sequence of SEQ ID NO: 21, and a light chain comprising an amino acid sequence at least 95% identical to the amino acid sequence of SEQ ID NO: 16; or
   (v) a heavy chain comprising an amino acid sequence at least 95% identical to the amino acid sequence of SEQ ID NO: 23, and a light chain comprising an amino acid sequence at least 95% identical to the amino acid sequence of SEQ ID NO: 10; or
   (vi) a heavy chain comprising an amino acid sequence at least 95% identical to the amino acid sequence of SEQ ID NO: 23, and a light chain comprising an amino acid sequence at least 95% identical to the amino acid sequence of SEQ ID NO: 16; or
   (vii) a heavy chain comprising an amino acid sequence at least 95% identical to the amino acid sequence of SEQ ID NO: 15, and a light chain comprising an amino acid sequence at least 95% identical to the amino acid sequence of SEQ ID NO: 26; or
   (viii) a heavy chain comprising an amino acid sequence at least 95% identical to the amino acid sequence of SEQ ID NO: 28, and a light chain comprising an amino acid sequence at least 95% identical to the amino acid sequence of SEQ ID NO: 10; or
   (ix) a heavy chain comprising an amino acid sequence at least 95% identical to the amino acid sequence of SEQ ID NO: 28, and a light chain comprising an amino acid sequence at least 95% identical to the amino acid sequence of SEQ ID NO: 16; or
   (x) a heavy chain comprising an amino acid sequence at least 95% identical to the amino acid sequence of SEQ ID NO: 31, and a light chain comprising an amino acid sequence at least 95% identical to the amino acid sequence of SEQ ID NO: 10; or
   (xi) a heavy chain comprising an amino acid sequence at least 95% identical to the amino acid sequence of SEQ ID NO: 31, and a light chain comprising an amino acid sequence at least 95% identical to the amino acid sequence of SEQ ID NO: 16; or
   (xii) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 37; or
   (xiii) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 40; or
   (xiv) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 43; or
   (xv) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 26; or
   (xvi) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 46; or
   (xvii) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 49; or
   (xviii) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 52; or
   (xix) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 55; or
   (xx) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 57; or
   (xxi) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 60; or
   (xxii) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 63; or
   (xxiii) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 66; or
   (xxiv) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 69; or
   (xxv) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 72; or
   (xxvi) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 74; or
   (xxvii) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 76; or
   (xxviii) a heavy chain comprising an amino acid sequence at least 95% identical to the amino acid sequence of SEQ ID NO: 80, and a light chain comprising an amino acid sequence at least 95% identical to the amino acid sequence of SEQ ID NO: 81; or
   (xxix) a heavy chain comprising an amino acid sequence at least 95% identical to the amino acid sequence of SEQ ID NO: 84, and a light chain comprising an amino acid sequence at least 95% identical to the amino acid sequence of SEQ ID NO: 81; or
   (xxx) a heavy chain comprising an amino acid sequence at least 95% identical to the amino acid sequence of SEQ ID NO: 28, and a light chain comprising an amino acid sequence at least 95% identical to the amino acid sequence of SEQ ID NO: 81; or
   (xxxi) a heavy chain comprising an amino acid sequence at least 95% identical to the amino acid sequence of SEQ ID NO: 87, and a light chain comprising an amino acid sequence at least 95% identical to the amino acid sequence of SEQ ID NO: 81; or
   (xxxii) a heavy chain comprising an amino acid sequence at least 95% identical to the amino acid sequence of SEQ ID NO: 90, and a light chain comprising an amino acid sequence at least 95% identical to the amino acid sequence of SEQ ID NO: 81; or
   (xxxiii) a heavy chain comprising an amino acid sequence at least 95% identical to the amino acid sequence of SEQ ID NO: 93, and a light chain comprising an amino acid sequence at least 95% identical to the amino acid sequence of SEQ ID NO: 81; or
   (xxxiv) a heavy chain comprising an amino acid sequence at least 95% identical to the amino acid sequence of SEQ ID NO: 96, and a light chain comprising an amino acid sequence at least 95% identical to the amino acid sequence of SEQ ID NO: 81; or
   (xxxv) a heavy chain comprising an amino acid sequence at least 95% identical to the amino acid sequence of SEQ ID NO: 98, and a light chain comprising an amino acid sequence at least 95% identical to the amino acid sequence of SEQ ID NO: 81; or
   (xxxvi) a heavy chain comprising an amino acid sequence at least 95% identical to the amino acid sequence of SEQ ID NO: 100, and a light chain comprising an amino acid sequence at least 95% identical to the amino acid sequence of SEQ ID NO: 81; or
   (xxxvii) a heavy chain comprising an amino acid sequence at least 95% identical to the amino acid sequence of SEQ ID NO: 102, and a light chain comprising an amino acid sequence at least 95% identical to the amino acid sequence of SEQ ID NO: 81; or
   (xxxviii) a heavy chain comprising an amino acid sequence at least 95% identical to the amino acid sequence of SEQ ID NO: 104, and a light chain comprising an amino acid sequence at least 95% identical to the amino acid sequence of SEQ ID NO: 81; or
   (xxxix) a heavy chain comprising an amino acid sequence at least 95% identical to the amino acid sequence of SEQ ID NO: 106, and a light chain comprising an amino acid sequence at least 95% identical to the amino acid sequence of SEQ ID NO: 81; or
   (xl) a heavy chain comprising an amino acid sequence at least 95% identical to the amino acid sequence of SEQ ID NO: 108, and a light chain comprising an amino acid sequence at least 95% identical to the amino acid sequence of SEQ ID NO: 81; or
   (xli) a heavy chain comprising an amino acid sequence at least 95% identical to the amino acid sequence of SEQ ID NO: 110, and a light chain comprising an amino acid sequence at least 95% identical to the amino acid sequence of SEQ ID NO: 81; or
   (xlii) a heavy chain comprising an amino acid sequence at least 95% identical to the amino acid sequence of SEQ ID NO: 112, and a light chain comprising an amino acid sequence at least 95% identical to the amino acid sequence of SEQ ID NO: 81; or
   (xliii) a heavy chain comprising an amino acid sequence at least 95% identical to the amino acid sequence of SEQ ID NO: 114, and a light chain comprising an amino acid sequence at least 95% identical to the amino acid sequence of SEQ ID NO: 81; or
   (xliv) a heavy chain comprising an amino acid sequence at least 95% identical to the amino acid sequence of SEQ ID NO: 116, and a light chain comprising an amino acid sequence at least 95% identical to the amino acid sequence of SEQ ID NO: 81; or
   (xlv) a heavy chain comprising an amino acid sequence at least 95% identical to the amino acid sequence of SEQ ID NO: 118, and a light chain comprising an amino acid sequence at least 95% identical to the amino acid sequence of SEQ ID NO: 81; or
   (xlvi) a heavy chain comprising an amino acid sequence at least 95% identical to the amino acid sequence of SEQ ID NO: 120, and a light chain comprising an amino acid sequence at least 95% identical to the amino acid sequence of SEQ ID NO: 81; or
   (xlvii) a heavy chain comprising an amino acid sequence at least 95% identical to the amino acid sequence of SEQ ID NO: 122, and a light chain comprising an amino acid sequence at least 95% identical to the amino acid sequence of SEQ ID NO: 81; or
   (xlviii) a heavy chain comprising an amino acid sequence at least 95% identical to the amino acid sequence of SEQ ID NO: 124, and a light chain comprising an amino acid sequence at least 95% identical to the amino acid sequence of SEQ ID NO: 81; or
   (xlix) a heavy chain comprising an amino acid sequence at least 95% identical to the amino acid sequence of SEQ ID NO: 127, and a light chain comprising an amino acid sequence at least 95% identical to the amino acid sequence of SEQ ID NO: 81; or
   (l) a heavy chain comprising an amino acid sequence at least 95% identical to the amino acid sequence of SEQ ID NO: 130, and a light chain comprising an amino acid sequence at least 95% identical to the amino acid sequence of SEQ ID NO: 81; or
   (li) a heavy chain comprising an amino acid sequence at least 95% identical to the amino acid sequence of SEQ ID NO: 133, and a light chain comprising an amino acid sequence at least 95% identical to the amino acid sequence of SEQ ID NO: 81; or
   (lii) a heavy chain comprising an amino acid sequence at least 95% identical to the amino acid sequence of SEQ ID NO: 135, and a light chain comprising an amino acid sequence at least 95% identical to the amino acid sequence of SEQ ID NO: 81; or
   (liii) a heavy chain comprising an amino acid sequence at least 95% identical to the amino acid sequence of SEQ ID NO: 137, and a light chain comprising an amino acid sequence at least 95% identical to the amino acid sequence of SEQ ID NO: 81; or
   (liv) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 10; or
   (lv) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 16; or
   (lvi) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 139; or
   (lvii) a heavy chain comprising an amino acid sequence at least 95% identical to the amino acid sequence of SEQ ID NO: 18, and a light chain comprising an amino acid sequence at least 95% identical to the amino acid sequence of SEQ ID NO: 81; or
   (lviii) a heavy chain comprising an amino acid sequence at least 95% identical to the amino acid sequence of SEQ ID NO: 18, and a light chain comprising an amino acid sequence at least 95% identical to the amino acid sequence of SEQ ID NO: 16; or
   (lix) a heavy chain comprising an amino acid sequence at least 95% identical to the amino acid sequence of SEQ ID NO: 18, and a light chain comprising an amino acid sequence at least 95% identical to the amino acid sequence of SEQ ID NO: 139; or
   (lx) a heavy chain comprising an amino acid sequence at least 95% identical to the amino acid sequence of SEQ ID NO: 18, and a light chain comprising an amino acid sequence at least 95% identical to the amino acid sequence of SEQ ID NO: 26; or
   (lxi) a heavy chain comprising an amino acid sequence at least 95% identical to the amino acid sequence of SEQ ID NO: 9, and a light chain comprising an amino acid sequence at least 95% identical to the amino acid sequence of SEQ ID NO: 81; or
   (lxii) a heavy chain comprising an amino acid sequence at least 95% identical to the amino acid sequence of SEQ ID NO: 9, and a light chain comprising an amino acid sequence at least 95% identical to the amino acid sequence of SEQ ID NO: 16; or
   (lxiii) a heavy chain comprising an amino acid sequence at least 95% identical to the amino acid sequence of SEQ ID NO: 9, and a light chain comprising an amino acid sequence at least 95% identical to the amino acid sequence of SEQ ID NO: 139; or
   (lxiv) a heavy chain comprising an amino acid sequence at least 95% identical to the amino acid sequence of SEQ ID NO: 9, and a light chain comprising an amino acid sequence at least 95% identical to the amino acid sequence of SEQ ID NO: 26; or
   (lxv) a heavy chain comprising an amino acid sequence at least 95% identical to the amino acid sequence of SEQ ID NO: 141, and a light chain comprising an amino acid sequence at least 95% identical to the amino acid sequence of SEQ ID NO: 81; or
   (lxvi) a heavy chain comprising an amino acid sequence at least 95% identical to the amino acid sequence of SEQ ID NO: 141, and a light chain comprising an amino acid sequence at least 95% identical to the amino acid sequence of SEQ ID NO: 10; or
   (lxvii) a heavy chain comprising an amino acid sequence at least 95% identical to the amino acid sequence of SEQ ID NO: 141, and a light chain comprising an amino acid sequence at least 95% identical to the amino acid sequence of SEQ ID NO: 16; or
   (lxviii) a heavy chain comprising an amino acid sequence at least 95% identical to the amino acid sequence of SEQ ID NO: 141, and a light chain comprising an amino acid sequence at least 95% identical to the amino acid sequence of SEQ ID NO: 139; or
   (lxix) a heavy chain comprising an amino acid sequence at least 95% identical to the amino acid sequence of SEQ ID NO: 141, and a light chain comprising an amino acid sequence at least 95% identical to the amino acid sequence of SEQ ID NO: 26; or
   (lxx) a heavy chain comprising an amino acid sequence at least 95% identical to the amino acid sequence of SEQ ID NO: 21, and a light chain comprising an amino acid sequence at least 95% identical to the amino acid sequence of SEQ ID NO: 81; or
   (lxxi) a heavy chain comprising an amino acid sequence at least 95% identical to the amino acid sequence of SEQ ID NO: 21, and a light chain comprising an amino acid sequence at least 95% identical to the amino acid sequence of SEQ ID NO: 10; or
   (lxxii) a heavy chain comprising an amino acid sequence at least 95% identical to the amino acid sequence of SEQ ID NO: 21, and a light chain comprising an amino acid sequence at least 95% identical to the amino acid sequence of SEQ ID NO: 139; or
   (lxxiii) a heavy chain comprising an amino acid sequence at least 95% identical to the amino acid sequence of SEQ ID NO: 21, and a light chain comprising an amino acid sequence at least 95% identical to the amino acid sequence of SEQ ID NO: 26; or
   (lxxiv) a heavy chain comprising an amino acid sequence at least 95% identical to the amino acid sequence of SEQ ID NO: 23, and a light chain comprising an amino acid sequence at least 95% identical to the amino acid sequence of SEQ ID NO: 81; or
   (lxxv) a heavy chain comprising an amino acid sequence at least 95% identical to the amino acid sequence of SEQ ID NO: 23, and a light chain comprising an amino acid sequence at least 95% identical to the amino acid sequence of SEQ ID NO: 139; or
   (lxxvi) a heavy chain comprising an amino acid sequence at least 95% identical to the amino acid sequence of SEQ ID NO: 23, and a light chain comprising an amino acid sequence at least 95% identical to the amino acid sequence of SEQ ID NO: 26; or
   (lxxvii) a heavy chain comprising an amino acid sequence at least 95% identical to the amino acid sequence of SEQ ID NO: 15, and a light chain comprising an amino acid sequence at least 95% identical to the amino acid sequence of SEQ ID NO: 81; or
   (lxxviii) a heavy chain comprising an amino acid sequence at least 95% identical to the amino acid sequence of SEQ ID NO: 15, and a light chain comprising an amino acid sequence at least 95% identical to the amino acid sequence of SEQ ID NO: 10; or
   (lxxix) a heavy chain comprising an amino acid sequence at least 95% identical to the amino acid sequence of SEQ ID NO: 15, and a light chain comprising an amino acid sequence at least 95% identical to the amino acid sequence of SEQ ID NO: 139; or
   (lxxx) a heavy chain comprising an amino acid sequence at least 95% identical to the amino acid sequence of SEQ ID NO: 28, and a light chain comprising an amino acid sequence at least 95% identical to the amino acid sequence of SEQ ID NO: 139; or
   (lxxxi) a heavy chain comprising an amino acid sequence at least 95% identical to the amino acid sequence of SEQ ID NO: 28, and a light chain comprising an amino acid sequence at least 95% identical to the amino acid sequence of SEQ ID NO: 26; or
   (lxxxii) a heavy chain comprising an amino acid sequence at least 95% identical to the amino acid sequence of SEQ ID NO: 31, and a light chain comprising an amino acid sequence at least 95% identical to the amino acid sequence of SEQ ID NO: 81; or
   (lxxxiii) a heavy chain comprising an amino acid sequence at least 95% identical to the amino acid sequence of SEQ ID NO: 31, and a light chain comprising an amino acid sequence at least 95% identical to the amino acid sequence of SEQ ID NO: 139; or
   (lxxxiv) a heavy chain comprising an amino acid sequence at least 95% identical to the amino acid sequence of SEQ ID NO: 31, and a light chain comprising an amino acid sequence at least 95% identical to the amino acid sequence of SEQ ID NO: 26; or
   (lxxxv) a heavy chain comprising an amino acid sequence at least 95% identical to the amino acid sequence of SEQ ID NO: 144, and a light chain comprising an amino acid sequence at least 95% identical to the amino acid sequence of SEQ ID NO: 81; or
   (lxxxvi) a heavy chain comprising an amino acid sequence at least 95% identical to the amino acid sequence of SEQ ID NO: 36, and a light chain comprising an amino acid sequence at least 95% identical to the amino acid sequence of SEQ ID NO: 81; or
   (lxxxvii) a heavy chain comprising an amino acid sequence at least 95% identical to the amino acid sequence of SEQ ID NO: 146, and a light chain comprising an amino acid sequence at least 95% identical to the amino acid sequence of SEQ ID NO: 81; or
   (lxxxviii) a heavy chain comprising an amino acid sequence at least 95% identical to the amino acid sequence of SEQ ID NO: 182, and a light chain comprising an amino acid sequence at least 95% identical to the amino acid sequence of SEQ ID NO: 81; or
   (lxxxix) a heavy chain comprising an amino acid sequence at least 95% identical to the amino acid sequence of SEQ ID NO: 144, and a light chain comprising an amino acid sequence at least 95% identical to the amino acid sequence of SEQ ID NO: 180; or
   (xc) a heavy chain comprising an amino acid sequence at least 95% identical to the amino acid sequence of SEQ ID NO: 146, and a light chain comprising an amino acid sequence at least 95% identical to the amino acid sequence of SEQ ID NO: 180; or
   (xci) a heavy chain comprising an amino acid sequence at least 95% identical to the amino acid sequence of SEQ ID NO: 182, and a light chain comprising an amino acid sequence at least 95% identical to the amino acid sequence of SEQ ID NO: 180; or
   (xcii) a heavy chain comprising an amino acid sequence at least 95% identical to the amino acid sequence of SEQ ID NO: 185, and a light chain comprising an amino acid sequence at least 95% identical to the amino acid sequence of SEQ ID NO: 81; or
   (xciii) a heavy chain comprising an amino acid sequence at least 95% identical to the amino acid sequence of SEQ ID NO: 187, and a light chain comprising an amino acid sequence at least 95% identical to the amino acid sequence of SEQ ID NO: 81; or
   (xciv) a heavy chain comprising an amino acid sequence at least 95% identical to the amino acid sequence of SEQ ID NO: 189, and a light chain comprising an amino acid sequence at least 95% identical to the amino acid sequence of SEQ ID NO: 81; or
   (xcv) a heavy chain comprising an amino acid sequence at least 95% identical to the amino acid sequence of SEQ ID NO: 185, and a light chain comprising an amino acid sequence at least 95% identical to the amino acid sequence of SEQ ID NO: 180; or
   (xcvi) a heavy chain comprising an amino acid sequence at least 95% identical to the amino acid sequence of SEQ ID NO: 187, and a light chain comprising an amino acid sequence at least 95% identical to the amino acid sequence of SEQ ID NO: 180; or
   (xcvii) a heavy chain comprising an amino acid sequence at least 95% identical to the amino acid sequence of SEQ ID NO: 189, and a light chain comprising an amino acid sequence at least 95% identical to the amino acid sequence of SEQ ID NO: 180.
47. The anti-LAYN antibody, or an antigen-binding fragment thereof, according to any one of the previous items, wherein the antibody or the antigen-binding fragment comprises a heavy chain comprising an amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146, 182, 185, 187 or 189; and a light chain comprising an amino acid sequence of any one of SEQ ID NOs 10, 16, 26, 37, 40, 43, 46, 49, 52, 55, 57, 60, 63, 66, 69, 72, 74, 76, 81, 139 or 180.
48. The anti-LAYN antibody, or an antigen-binding fragment thereof, according to any one of the previous items, wherein the antibody or the antigen-binding fragment comprises a heavy chain comprising an amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146 or 182; and a light chain comprising an amino acid sequence of any one of SEQ ID NOs 10, 16, 26, 37, 40, 43, 46, 49, 52, 55, 57, 60, 63, 66, 69, 72, 74, 76, 81, 139 or 180.
49. The anti-LAYN antibody, or an antigen-binding fragment thereof, according to any one of the previous items, wherein the antibody or the antigen-binding fragment comprises (or consists of):
   (i) a heavy chain comprising the amino acid sequence of SEQ ID NO: 9, and a light chain comprising the amino acid sequence of SEQ ID NO: 10; or
   (ii) a heavy chain comprising the amino acid sequence of SEQ ID NO: 15, and a light chain comprising the amino acid sequence of SEQ ID NO: 16; or
   (iii) a heavy chain comprising the amino acid sequence of SEQ ID NO: 18, and a light chain comprising the amino acid sequence of SEQ ID NO: 10; or
   (iv) a heavy chain comprising the amino acid sequence of SEQ ID NO: 21, and a light chain comprising the amino acid sequence of SEQ ID NO: 16; or
   (v) a heavy chain comprising the amino acid sequence of SEQ ID NO: 23, and a light chain comprising the amino acid sequence of SEQ ID NO: 10; or
   (vi) a heavy chain comprising the amino acid sequence of SEQ ID NO: 23, and a light chain comprising the amino acid sequence of SEQ ID NO: 16; or
   (vii) a heavy chain comprising the amino acid sequence of SEQ ID NO: 15, and a light chain comprising the amino acid sequence of SEQ ID NO: 26; or
   (viii) a heavy chain comprising the amino acid sequence of SEQ ID NO: 28, and a light chain comprising the amino acid sequence of SEQ ID NO: 10; or
   (ix) a heavy chain comprising the amino acid sequence of SEQ ID NO: 28, and a light chain comprising the amino acid sequence of SEQ ID NO: 16; or
   (x) a heavy chain comprising the amino acid sequence of SEQ ID NO: 31, and a light chain comprising the amino acid sequence of SEQ ID NO: 10; or
   (xi) a heavy chain comprising the amino acid sequence of SEQ ID NO: 31, and a light chain comprising the amino acid sequence of SEQ ID NO: 16; or
   (xii) a heavy chain comprising the amino acid sequence of SEQ ID NO: 36, and a light chain comprising the amino acid sequence of SEQ ID NO: 37; or
   (xiii) a heavy chain comprising the amino acid sequence of SEQ ID NO: 36, and a light chain comprising the amino acid sequence of SEQ ID NO: 40; or
   (xiv) a heavy chain comprising the amino acid sequence of SEQ ID NO: 36, and a light chain comprising the amino acid sequence of SEQ ID NO: 43; or
   (xv) a heavy chain comprising the amino acid sequence of SEQ ID NO: 36, and a light chain comprising the amino acid sequence of SEQ ID NO: 26; or
   (xvi) a heavy chain comprising the amino acid sequence of SEQ ID NO: 36, and a light chain comprising the amino acid sequence of SEQ ID NO: 46; or
   (xvii) a heavy chain comprising the amino acid sequence of SEQ ID NO: 36, and a light chain comprising the amino acid sequence of SEQ ID NO: 49; or
   (xviii) a heavy chain comprising the amino acid sequence of SEQ ID NO: 36, and a light chain comprising the amino acid sequence of SEQ ID NO: 52; or
   (xix) a heavy chain comprising the amino acid sequence of SEQ ID NO: 36, and a light chain comprising the amino acid sequence of SEQ ID NO: 55; or
   (xx a heavy chain comprising the amino acid sequence of SEQ ID NO: 36, and a light chain comprising the amino acid sequence of SEQ ID NO: 57; or
   (xxi) a heavy chain comprising the amino acid sequence of SEQ ID NO: 36, and a light chain comprising the amino acid sequence of SEQ ID NO: 60; or
   (xxii) a heavy chain comprising the amino acid sequence of SEQ ID NO: 36, and a light chain comprising the amino acid sequence of SEQ ID NO: 63; or
   (xxiii) a heavy chain comprising the amino acid sequence of SEQ ID NO: 36, and a light chain comprising the amino acid sequence of SEQ ID NO: 66; or
   (xxiv) a heavy chain comprising the amino acid sequence of SEQ ID NO: 36, and a light chain comprising the amino acid sequence of SEQ ID NO: 69; or
   (xxv) a heavy chain comprising the amino acid sequence of SEQ ID NO: 36, and a light chain comprising the amino acid sequence of SEQ ID NO: 72; or
   (xxvi) a heavy chain comprising the amino acid sequence of SEQ ID NO: 36, and a light chain comprising the amino acid sequence of SEQ ID NO: 74; or
   (xxvii) a heavy chain comprising the amino acid sequence of SEQ ID NO: 36, and a light chain comprising the amino acid sequence of SEQ ID NO: 76; or
   (xxviii) a heavy chain comprising the amino acid sequence of SEQ ID NO: 80, and a light chain comprising the amino acid sequence of SEQ ID NO: 81; or
   (xxix) a heavy chain comprising the amino acid sequence of SEQ ID NO: 84, and a light chain comprising the amino acid sequence of SEQ ID NO: 81; or
   (xxx) a heavy chain comprising the amino acid sequence of SEQ ID NO: 28, and a light chain comprising the amino acid sequence of SEQ ID NO: 81; or
   (xxxi) a heavy chain comprising the amino acid sequence of SEQ ID NO: 87, and a light chain comprising the amino acid sequence of SEQ ID NO: 81; or
   (xxxii) a heavy chain comprising the amino acid sequence of SEQ ID NO: 90, and a light chain comprising the amino acid sequence of SEQ ID NO: 81; or
   (xxxiii) a heavy chain comprising the amino acid sequence of SEQ ID NO: 93, and a light chain comprising the amino acid sequence of SEQ ID NO: 81; or
   (xxxiv) a heavy chain comprising the amino acid sequence of SEQ ID NO: 96, and a light chain comprising the amino acid sequence of SEQ ID NO: 81; or
   (xxxv) a heavy chain comprising the amino acid sequence of SEQ ID NO: 98, and a light chain comprising the amino acid sequence of SEQ ID NO: 81; or
   (xxxvi) a heavy chain comprising the amino acid sequence of SEQ ID NO: 100, and a light chain comprising the amino acid sequence of SEQ ID NO: 81; or
   (xxxvii) a heavy chain comprising the amino acid sequence of SEQ ID NO: 102, and a light chain comprising the amino acid sequence of SEQ ID NO: 81; or
   (xxxviii) a heavy chain comprising the amino acid sequence of SEQ ID NO: 104, and a light chain comprising the amino acid sequence of SEQ ID NO: 81; or
   (xxxix) a heavy chain comprising the amino acid sequence of SEQ ID NO: 106, and a light chain comprising the amino acid sequence of SEQ ID NO: 81; or
   (xl) a heavy chain comprising the amino acid sequence of SEQ ID NO: 108, and a light chain comprising the amino acid sequence of SEQ ID NO: 81; or
   (xli) a heavy chain comprising the amino acid sequence of SEQ ID NO: 110, and a light chain comprising the amino acid sequence of SEQ ID NO: 81; or
   (xlii) a heavy chain comprising the amino acid sequence of SEQ ID NO: 112, and a light chain comprising the amino acid sequence of SEQ ID NO: 81; or
   (xliii) a heavy chain comprising the amino acid sequence of SEQ ID NO: 114, and a light chain comprising the amino acid sequence of SEQ ID NO: 81; or
   (xliv) a heavy chain comprising the amino acid sequence of SEQ ID NO: 116, and a light chain comprising the amino acid sequence of SEQ ID NO: 81; or
   (xlv) a heavy chain comprising the amino acid sequence of SEQ ID NO: 118, and a light chain comprising the amino acid sequence of SEQ ID NO: 81; or
   (xlvi) a heavy chain comprising the amino acid sequence of SEQ ID NO: 120, and a light chain comprising the amino acid sequence of SEQ ID NO: 81; or
   (xlvii) a heavy chain comprising the amino acid sequence of SEQ ID NO: 122, and a light chain comprising the amino acid sequence of SEQ ID NO: 81; or
   (xlviii) a heavy chain comprising the amino acid sequence of SEQ ID NO: 124, and a light chain comprising the amino acid sequence of SEQ ID NO: 81; or
   (xlix) a heavy chain comprising the amino acid sequence of SEQ ID NO: 127, and a light chain comprising the amino acid sequence of SEQ ID NO: 81; or
   (l) a heavy chain comprising the amino acid sequence of SEQ ID NO: 130, and a light chain comprising the amino acid sequence of SEQ ID NO: 81; or
   (li) a heavy chain comprising the amino acid sequence of SEQ ID NO: 133, and a light chain comprising the amino acid sequence of SEQ ID NO: 81; or
   (lii) a heavy chain comprising the amino acid sequence of SEQ ID NO: 135, and a light chain comprising the amino acid sequence of SEQ ID NO: 81; or
   (liii) a heavy chain comprising the amino acid sequence of SEQ ID NO: 137, and a light chain comprising the amino acid sequence of SEQ ID NO: 81; or
   (liv) a heavy chain comprising the amino acid sequence of SEQ ID NO: 36, and a light chain comprising the amino acid sequence of SEQ ID NO: 10; or
   (lv) a heavy chain comprising the amino acid sequence of SEQ ID NO: 36, and a light chain comprising the amino acid sequence of SEQ ID NO: 16; or
   (lvi) a heavy chain comprising the amino acid sequence of SEQ ID NO: 36, and a light chain comprising the amino acid sequence of SEQ ID NO: 139; or
   (lvii) a heavy chain comprising the amino acid sequence of SEQ ID NO: 18, and a light chain comprising the amino acid sequence of SEQ ID NO: 81; or
   (lviii) a heavy chain comprising the amino acid sequence of SEQ ID NO: 18, and a light chain comprising the amino acid sequence of SEQ ID NO: 16; or
   (lix) a heavy chain comprising the amino acid sequence of SEQ ID NO: 18, and a light chain comprising the amino acid sequence of SEQ ID NO: 139; or
   (lx) a heavy chain comprising the amino acid sequence of SEQ ID NO: 18, and a light chain comprising the amino acid sequence of SEQ ID NO: 26; or
   (lxi) a heavy chain comprising the amino acid sequence of SEQ ID NO: 9, and a light chain comprising the amino acid sequence of SEQ ID NO: 81; or
   (lxii) a heavy chain comprising the amino acid sequence of SEQ ID NO: 9, and a light chain comprising the amino acid sequence of SEQ ID NO: 16; or
   (lxiii) a heavy chain comprising the amino acid sequence of SEQ ID NO: 9, and a light chain comprising the amino acid sequence of SEQ ID NO: 139; or
   (lxiv) a heavy chain comprising the amino acid sequence of SEQ ID NO: 9, and a light chain comprising the amino acid sequence of SEQ ID NO: 26; or
   (lxv) a heavy chain comprising the amino acid sequence of SEQ ID NO: 141, and a light chain comprising the amino acid sequence of SEQ ID NO: 81; or
   (lxvi) a heavy chain comprising the amino acid sequence of SEQ ID NO: 141, and a light chain comprising the amino acid sequence of SEQ ID NO: 10; or
   (lxvii) a heavy chain comprising the amino acid sequence of SEQ ID NO: 141, and a light chain comprising the amino acid sequence of SEQ ID NO: 16; or
   (lxviii) a heavy chain comprising the amino acid sequence of SEQ ID NO: 141, and a light chain comprising the amino acid sequence of SEQ ID NO: 139; or
   (lxix) a heavy chain comprising the amino acid sequence of SEQ ID NO: 141, and a light chain comprising the amino acid sequence of SEQ ID NO: 26; or
   (lxx) a heavy chain comprising the amino acid sequence of SEQ ID NO: 21, and a light chain comprising the amino acid sequence of SEQ ID NO: 81; or
   (lxxi) a heavy chain comprising the amino acid sequence of SEQ ID NO: 21, and a light chain comprising the amino acid sequence of SEQ ID NO: 10; or
   (lxxii) a heavy chain comprising the amino acid sequence of SEQ ID NO: 21, and a light chain comprising the amino acid sequence of SEQ ID NO: 139; or
   (lxxiii) a heavy chain comprising the amino acid sequence of SEQ ID NO: 21, and a light chain comprising the amino acid sequence of SEQ ID NO: 26; or
   (lxxiv) a heavy chain comprising the amino acid sequence of SEQ ID NO: 23, and a light chain comprising the amino acid sequence of SEQ ID NO: 81; or
   (lxxv) a heavy chain comprising the amino acid sequence of SEQ ID NO: 23, and a light chain comprising the amino acid sequence of SEQ ID NO: 139; or
   (lxxvi) a heavy chain comprising the amino acid sequence of SEQ ID NO: 23, and a light chain comprising the amino acid sequence of SEQ ID NO: 26; or
   (lxxvii) a heavy chain comprising the amino acid sequence of SEQ ID NO: 15, and a light chain comprising the amino acid sequence of SEQ ID NO: 81; or
   (lxxviii) a heavy chain comprising the amino acid sequence of SEQ ID NO: 15, and a light chain comprising the amino acid sequence of SEQ ID NO: 10; or
   (lxxix) a heavy chain comprising the amino acid sequence of SEQ ID NO: 15, and a light chain comprising the amino acid sequence of SEQ ID NO: 139; or
   (lxxx) a heavy chain comprising the amino acid sequence of SEQ ID NO: 28, and a light chain comprising the amino acid sequence of SEQ ID NO: 139; or
   (lxxxi) a heavy chain comprising the amino acid sequence of SEQ ID NO: 28, and a light chain comprising the amino acid sequence of SEQ ID NO: 26; or
   (lxxxii) a heavy chain comprising the amino acid sequence of SEQ ID NO: 31, and a light chain comprising the amino acid sequence of SEQ ID NO: 81; or
   (lxxxiii) a heavy chain comprising the amino acid sequence of SEQ ID NO: 31, and a light chain comprising the amino acid sequence of SEQ ID NO: 139; or
   (lxxxiv) a heavy chain comprising the amino acid sequence of SEQ ID NO: 31, and a light chain comprising the amino acid sequence of SEQ ID NO: 26; or
   (lxxxv) a heavy chain comprising the amino acid sequence of SEQ ID NO: 144, and a light chain comprising the amino acid sequence of SEQ ID NO: 81; or
   (lxxxvi) a heavy chain comprising the amino acid sequence of SEQ ID NO: 36, and a light chain comprising the amino acid sequence of SEQ ID NO: 81; or
   (lxxxvii) a heavy chain comprising the amino acid sequence of SEQ ID NO: 146, and a light chain comprising the amino acid sequence of SEQ ID NO: 81; or
   (lxxxviii) a heavy chain comprising the amino acid sequence of SEQ ID NO: 182, and a light chain comprising the amino acid sequence of SEQ ID NO: 81; or
   (lxxxix) a heavy chain comprising the amino acid sequence of SEQ ID NO: 144, and a light chain comprising the amino acid sequence of SEQ ID NO: 180; or
   (xc) a heavy chain comprising the amino acid sequence of SEQ ID NO: 146, and a light chain comprising the amino acid sequence of SEQ ID NO: 180; or
   (xci) a heavy chain comprising the amino acid sequence of SEQ ID NO: 182, and a light chain comprising the amino acid sequence of SEQ ID NO: 180; or
   (xcii) a heavy chain comprising the amino acid sequence of SEQ ID NO: 185, and a light chain comprising the amino acid sequence of SEQ ID NO: 81; or
   (xciii) a heavy chain comprising the amino acid sequence of SEQ ID NO: 187, and a light chain comprising the amino acid sequence of SEQ ID NO: 81; or
   (xciv) a heavy chain comprising the amino acid sequence of SEQ ID NO: 189, and a light chain comprising the amino acid sequence of SEQ ID NO: 81; or
   (xcv) a heavy chain comprising the amino acid sequence of SEQ ID NO: 185, and a light chain comprising the amino acid sequence of SEQ ID NO: 180; or
   (xcvi) a heavy chain comprising the amino acid sequence of SEQ ID NO: 187, and a light chain comprising the amino acid sequence of SEQ ID NO: 180; or
   (xcvii) a heavy chain comprising the amino acid sequence of SEQ ID NO: 189, and a light chain comprising the amino acid sequence of SEQ ID NO: 180.
50. The anti-LAYN antibody, or an antigen-binding fragment thereof, according to any one of items 1 to 12, wherein the antibody or the antigen-binding fragment comprises:
   a heavy chain variable region comprising:
      a heavy chain complementary determining region 1 (HCDR1) having the amino acid sequence of SEQ ID NO: 1;
      a heavy chain complementary determining region 2 (HCDR2) having the amino acid sequence of NIX₂PX₃SGX₄TNYX₅X₆KFX₇X₈ (SEQ ID NO: 207), wherein
         X₂ is Y;
         X₃ is S or G;
         X₄ is T or S;
         X₅ is A;
         X₆ is Q;
         X₇ is Q; and
         X₈ is G; and
      a heavy chain complementary determining region 3 (HCDR3) having the amino acid sequence of X₉X₁₀YX₁₁RX₁₂AX₁₃X₁₄X₁₅ (SEQ ID NO: 208), wherein
         X₉ is E;
         X₁₀ is R;
         X₁₁ is G;
         X₁₂ is N;
         X₁₃ is A or M;
         X₁₄ is E or D; and
         X₁₅ is Y; and
   a light chain variable region comprising
      a light chain complementary determining region 1 (LCDR1) having the amino acid sequence of X₁₆ASQX₁₇₁X₁₈X₁₉X₂₀LA (SEQ ID NO: 209), wherein
         X₁₆ is R or L;
         X₁₇ is G or T;
         X₁₈ is G;
         X₁₉ is S or T; and
         X₂₀ is W;
      a light chain complementary determining region 2 (LCDR2) having the amino acid sequence of SEQ ID NO: 5; and
      a light chain complementary determining region 3 (LCDR3) having the amino acid sequence of SEQ ID NO: 6.
51. An anti-LAYN antibody, or an antigen-binding fragment thereof, comprising:
   a heavy chain variable region comprising:
      a heavy chain complementary determining region 1 (HCDR1) having the amino acid sequence of SEQ ID NO: 1;
      a heavy chain complementary determining region 2 (HCDR2) having the amino acid sequence of NIX₂PX₃SGX₄TNYX₅X₆KFX₇X₈ (SEQ ID NO: 207), wherein
         X₂ is Y;
         X₃ is S or G;
         X₄ is T or S;
         X₅ is A;
         X₆ is Q;
         X₇ is Q; and
         X₈ is G; and
      a heavy chain complementary determining region 3 (HCDR3) having the amino acid sequence of X₉X₁₀YX₁₁RX₁₂AX₁₃X₁₄X₁₅ (SEQ ID NO: 208), wherein
         X₉ is E;
         X₁₀ is R;
         X₁₁ is G;
         X₁₂ is N;
         X₁₃ is A or M;
         X₁₄ is E or D; and
         X₁₅ is Y; and
   a light chain variable region comprising
      a light chain complementary determining region 1 (LCDR1) having the amino acid sequence of X₁₆ASQX₁₇₁X₁₈X₁₉X₂₀LA (SEQ ID NO: 209), wherein
         X₁₆ is R or L;
         X₁₇ is G or T;
         X₁₈ is G;
         X₁₉ is S or T; and
         X₂₀ is W;
      a light chain complementary determining region 2 (LCDR2) having the amino acid sequence of SEQ ID NO: 5; and
   a light chain complementary determining region 3 (LCDR3) having the amino acid sequence of SEQ ID NO: 6.
52. The anti-LAYN antibody, or an antigen-binding fragment thereof, according to item 50 or 51, wherein the antibody or the antigen-binding fragment comprises:
   a heavy chain variable region comprising an HCDR1 having the amino acid sequence of SEQ ID NO: 1, an HCDR2 having the amino acid sequence of any one of SEQ ID NOs 2, 11, or 19, and an HCDR3 having the amino acid sequence of any one of SEQ ID NOs 3, 32, or 142; and
   a light chain variable region comprising an LCDR1 having the amino acid sequence of any one of SEQ ID NOs 4, 12, or 24, an LCDR2 having the amino acid sequence of SEQ ID NO: 5, and an LCDR3 having the amino acid sequence of SEQ ID NO: 6.
53. The anti-LAYN antibody, or an antigen-binding fragment thereof, according to any one of items 50 to 52, wherein the antibody or the antigen-binding fragment comprises:
   a heavy chain variable region comprising:
      an HCDR1 having the amino acid sequence of SEQ ID NO: 1, an HCDR2 having the amino acid sequence of SEQ ID NO: 2 and an HCDR3 having the amino acid sequence of SEQ ID NO: 3, or
      an HCDR1 having the amino acid sequence of SEQ ID NO: 1, an HCDR2 having the amino acid sequence of SEQ ID NO: 11 and an HCDR3 having the amino acid sequence of SEQ ID NO: 3, or
      an HCDR1 having the amino acid sequence of SEQ ID NO: 1, an HCDR2 having the amino acid sequence of SEQ ID NO: 19 and an HCDR3 having the amino acid sequence of SEQ ID NO: 142, or
      an HCDR1 having the amino acid sequence of SEQ ID NO: 1, an HCDR2 having the amino acid sequence of SEQ ID NO: 19 and an HCDR3 having the amino acid sequence of SEQ ID NO: 32; and
   a light chain variable region comprising:
      an LCDR1 having the amino acid sequence of SEQ ID NO: 4, an LCDR2 having the amino acid sequence of SEQ ID NO: 5 and an LCDR3 having the amino acid sequence of SEQ ID NO: 6, or
      an LCDR1 having the amino acid sequence of SEQ ID NO: 12, an LCDR2 having the amino acid sequence of SEQ ID NO: 5 and an LCDR3 having the amino acid sequence of SEQ ID NO: 6, or
      an LCDR1 having the amino acid sequence of SEQ ID NO: 24, an LCDR2 having the amino acid sequence of SEQ ID NO: 5 and an LCDR3 having the amino acid sequence of SEQ ID NO: 6.
54. The anti-LAYN antibody, or an antigen-binding fragment thereof, according to any one of items 50 to 53, wherein the antibody or the antigen-binding fragment comprises:
   a heavy chain variable region (VH) comprising the amino acid sequence of QVQLX₂₆QX₂₇GX₂₈EX₂₉X₃₀X₃₁PGASVKX₃₂SCKASGYTFTX₁YWMDWVX₃₃QX₃₄PG X₃₅GLEWX₃₆GNIX₂PX₃SGX₄TNYX₅X₆KFX₇X₈X₃₇X₃₈TX₃₉TX₄₀DX₄₁X₄₂X₄₃X₄₄TX₄₅YM X₄₆LSX₄₇LX₄₈SX₄₉DX₅₀AX₅₁YYCX₅₂RX₉X₁₀YX₁₁RX₁₂AX₁₃X₁₄X₁₅WGQGTX₅₃VTVSS
   (SEQ ID NO: 212), wherein
   X₁ is S, X₂ is Y, X₃ is Sor G, X₄ is T or S, X₅ is A, X₆ is Q, X₇ is Q, X₈ is G, X₉ is E, X₁₀ is R, X₁₁ is G, X₁₂ is N, X₁₃ is A or M, X₁₄ is E or D, X₁₅ is Y, X₂₆ is V, X₂₇ is S, X₂₈ is A or S, X₂₉ is V, X₃₀ is K, X₃₁ is K, X₃₂ is V, X₃₃ is R, X₃₄ is A, X₃₅ is Q, X₃₆ is M, X₃₇ is R, X₃₈ is V or A, X₃₉ is M or L, X₄₀ is V, X₄₁ is K, X₄₂ is S, X₄₃ is T, X₄₄ is S, X₄₅ is A, X₄₆ is E, X₄₇ is S, X₄₈ is R, X₄₉ is E, X₅₀ is T, X₅₁ is V, X₅₂ is A or T, and X₅₃ is L; and
   a light chain variable region (VL) comprising the amino acid sequence of DIQMTQSPX₅₄SX₅₅SASX₅₆GX₅₇X₅₈VTITCX₁₆ASQX₁₇₁X₁₈X₁₉X₂₀LAWYQQKPGKX₅₉PX₆₀LLIYAAX₂₁SLX₂₂X₂₃GVPSRFSGSGSGTX₆₁FX₆₂X₆₃X₆₄ISSLQX₆₅EDFX₆₆X₆₇YY CQQX₂₄YSX₂₅PLTFGGGTKLEIK
   (SEQ ID NO: 213), wherein
   X₁₆ is R or L, X₁₇ is G or T, X₁₈ is G, X₁₉ is S or T, X₂₀ is W, X₂₁ is T, X₂₂ is A, X₂₃ is D, X₂₄ is L, X₂₅ is T, X₅₄ is S, X₅₅ is V, X₅₆ is V, X₅₇ is D, X₅₈ is R, X₅₉ is A or S, X₆₀ is K, X₆₁ is D, X₆₂ is T, X₆₃ is L or F, X₆₄ is T, X₆₅ is P, X₆₆ is A, and X₆₇ is T.
55. An anti-LAYN antibody, or an antigen-binding fragment thereof, comprising:
   a heavy chain variable region (VH) comprising the amino acid sequence of QVQLX₂₆QX₂₇GX₂₈EX₂₉X₃₀X₃₁PGASVKX₃₂SCKASGYTFTX₁YWMDWVX₃₃QX₃₄PG X₃₅GLEWX₃₆GNIX₂PX₃SGX₄TNYX₅X₆KFX₇X₈X₃₇X₃₈TX₃₉TX₄₀DX₄₁X₄₂X₄₃X₄₄TX₄₅YM X₄₆LSX₄₇LX₄₈SX₄₉DX₅₀AX₅₁YYCX₅₂RX₉X₁₀YX₁₁RX₁₂AX₁₃X₁₄X₁₅WGQGTX₅₃VTVSS
   (SEQ ID NO: 212), wherein
   X₁ is S, X₂ is Y, X₃ is S or G, X₄ is T or S, X₅ is A, X₆ is Q, X₇ is Q, X₈ is G, X₉ is E, X₁₀ is R, X₁₁ is G, X₁₂ is N, X₁₃ is A or M, X₁₄ is E or D, X₁₅ is Y, X₂₆ is V, X₂₇ is S, X₂₈ is A or S, X₂₉ is V, X₃₀ is K, X₃₁ is K, X₃₂ is V, X₃₃ is R, X₃₄ is A, X₃₅ is Q, X₃₆ is M, X₃₇ is R, X₃₈ is V or A, X₃₉ is M or L, X₄₀ is V, X₄₁ is K, X₄₂ is S, X₄₃ is T, X₄₄ is S, X₄₅ is A, X₄₆ is E, X₄₇ is S, X₄₈ is R, X₄₉ is E, X₅₀ is T, X₅₁ is V, X₅₂ is A or T, and X₅₃ is L; and
   a light chain variable region (VL) comprising the amino acid sequence of DIQMTQSPX₅₄SX₅₅SASX₅₆GX₅₇X₅₈VTITCX₁₆ASQX₁₇₁X₁₈X₁₉X₂₀LAWYQQKPGKX₅₉PX₆₀LLIYAAX₂₁SLX₂₂X₂₃GVPSRFSGSGSGTX₆₁FX₆₂X₆₃X₆₄ISSLQX₆₅EDFX₆₆X₆₇YY CQQX₂₄YSX₂₅PLTFGGGTKLEIK
   (SEQ ID NO: 213), wherein
   X₁₆ is R or L, X₁₇ is G or T, X₁₈ is G, X₁₉ is S or T, X₂₀ is W, X₂₁ is T, X₂₂ is A, X₂₃ is D, X₂₄ is L, X₂₅ is T, X₅₄ is S, X₅₅ is V, X₅₆ is V, X₅₇ is D, X₅₈ is R, X₅₉ is A or S, X₆₀ is K, X₆₁ is D, X₆₂ is T, X₆₃ is L or F, X₆₄ is T, X₆₅ is P, X₆₆ is A, and X₆₇ is T.
56. The anti-LAYN antibody, or an antigen-binding fragment thereof, according to any one of items 50 to 55, wherein the antibody or the antigen-binding fragment comprises: a heavy chain variable region comprising an amino acid sequence at least 80% identical to the amino acid sequence of any one of SEQ ID NOs 7, 13, 34 or 143; and a light chain variable region comprising an amino acid sequence at least 80% identical to the amino acid sequence of any one of SEQ ID NOs 8, 14 or 79.
57. An anti-LAYN antibody, or an antigen-binding fragment thereof, comprising: a heavy chain variable region comprising an amino acid sequence at least 80% identical to the amino acid sequence of any one of SEQ ID NOs 7, 13, 34 or 143; and a light chain variable region comprising an amino acid sequence at least 80% identical to the amino acid sequence of any one of SEQ ID NOs 8, 14 or 79.
58. The anti-LAYN antibody, or an antigen-binding fragment thereof, according to any one of items 50 to 57, wherein the antibody or the antigen-binding fragment comprises: a heavy chain variable region comprising an amino acid sequence at least 85% identical to the amino acid sequence of any one of SEQ ID NOs 7, 13, 34 or 143; and a light chain variable region comprising an amino acid sequence at least 85% identical to the amino acid sequence of any one of SEQ ID NOs 8, 14 or 79.
59. The anti-LAYN antibody, or an antigen-binding fragment thereof, according to item 58, wherein the antibody or the antigen-binding fragment comprises: a heavy chain variable region comprising an amino acid sequence at least 90% identical to the amino acid sequence of any one of SEQ ID NOs 7, 13, 34 or 143; and a light chain variable region comprising an amino acid sequence at least 90% identical to the amino acid sequence of any one of SEQ ID NOs 8, 14 or 79.
60. The anti-LAYN antibody, or an antigen-binding fragment thereof, according to item 59, wherein the antibody or the antigen-binding fragment comprises: a heavy chain variable region comprising an amino acid sequence at least 95% identical to the amino acid sequence of any one of SEQ ID NOs 7, 13, 34 or 143; and a light chain variable region comprising an amino acid sequence at least 95% identical to the amino acid sequence of any one of SEQ ID NOs 8, 14 or 79.
61. The anti-LAYN antibody, or an antigen-binding fragment thereof, according to item 60, wherein the antibody or the antigen-binding fragment comprises: a heavy chain variable region comprising an amino acid sequence of any one of SEQ ID NOs 7, 13, 34 or 143; and a light chain variable region comprising an amino acid sequence of any one of SEQ ID NOs 8, 14 or 79.
62. The anti-LAYN antibody, or an antigen-binding fragment thereof, according to any one of the previous items, wherein the antibody or the antigen-binding fragment comprises: a heavy chain variable region comprising an HCDR1 having the amino acid sequence of SEQ ID NO: 1, an HCDR2 having the amino acid sequence of SEQ ID NO: 2, and an HCDR3 having the amino acid sequence of SEQ ID NO: 3; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6.
63. The anti-LAYN antibody, or an antigen-binding fragment thereof, according to item 62, wherein the antibody or the antigen-binding fragment comprises a heavy chain variable region comprising an amino acid sequence at least 80% identical to the amino acid sequence of SEQ ID NO: 7; and a light chain variable region comprising an amino acid sequence at least 80% identical to the amino acid sequence of SEQ ID NO: 8.
64. The anti-LAYN antibody, or an antigen-binding fragment thereof, according to item 62, wherein the antibody or the antigen-binding fragment comprises a heavy chain variable region comprising an amino acid sequence at least 85% identical to the amino acid sequence of SEQ ID NO: 7; and a light chain variable region comprising an amino acid sequence at least 85% identical to the amino acid sequence of SEQ ID NO: 8.
65. The anti-LAYN antibody, or an antigen-binding fragment thereof, according to item 62, wherein the antibody or the antigen-binding fragment comprises a heavy chain variable region comprising an amino acid sequence at least 90% identical to the amino acid sequence of SEQ ID NO: 7; and a light chain variable region comprising an amino acid sequence at least 90% identical to the amino acid sequence of SEQ ID NO: 8.
66. The anti-LAYN antibody, or an antigen-binding fragment thereof, according to item 62, wherein the antibody or the antigen-binding fragment comprises a heavy chain variable region comprising an amino acid sequence at least 95% identical to the amino acid sequence of SEQ ID NO: 7; and a light chain variable region comprising an amino acid sequence at least 95% identical to the amino acid sequence of SEQ ID NO: 8.
67. The anti-LAYN antibody, or an antigen-binding fragment thereof, according to item 62, wherein the antibody or the antigen-binding fragment comprises a heavy chain variable region comprising the amino acid sequence of SEQ ID NO: 7; and a light chain variable region comprising the amino acid sequence of SEQ ID NO: 8.
68. The anti-LAYN antibody, or an antigen-binding fragment thereof, according to any one of the previous items, wherein the antibody or the antigen-binding fragment comprises (or consists of) a heavy chain comprising the amino acid sequence of SEQ ID NO: 9; and a light chain comprising the amino acid sequence of SEQ ID NO: 10.
69. The anti-LAYN antibody, or an antigen-binding fragment thereof, according to any one of items 1 to 61, wherein the antibody or the antigen-binding fragment comprises:
   a heavy chain variable region comprising an HCDR1 having the amino acid sequence of SEQ ID NO: 1, an HCDR2 having the amino acid sequence of SEQ ID NO: 11, and an HCDR3 having the amino acid sequence of SEQ ID NO: 3; and
   a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6.
70. The anti-LAYN antibody, or an antigen-binding fragment thereof, according to item 69, wherein the antibody or the antigen-binding fragment comprises a heavy chain variable region comprising an amino acid sequence at least 80% identical to the amino acid sequence of SEQ ID NO: 13; and a light chain variable region comprising an amino acid sequence at least 80% identical to the amino acid sequence of SEQ ID NO: 14.
71. The anti-LAYN antibody, or an antigen-binding fragment thereof, according to item 69, wherein the antibody or the antigen-binding fragment comprises a heavy chain variable region comprising an amino acid sequence at least 85% identical to the amino acid sequence of SEQ ID NO: 13; and a light chain variable region comprising an amino acid sequence at least 85% identical to the amino acid sequence of SEQ ID NO: 14.
72. The anti-LAYN antibody, or an antigen-binding fragment thereof, according to item 69, wherein the antibody or the antigen-binding fragment comprises a heavy chain variable region comprising an amino acid sequence at least 90% identical to the amino acid sequence of SEQ ID NO: 13; and a light chain variable region comprising an amino acid sequence at least 90% identical to the amino acid sequence of SEQ ID NO: 14.
73. The anti-LAYN antibody, or an antigen-binding fragment thereof, according to item 69, wherein the antibody or the antigen-binding fragment comprises a heavy chain variable region comprising an amino acid sequence at least 95% identical to the amino acid sequence of SEQ ID NO: 13; and a light chain variable region comprising an amino acid sequence at least 95% identical to the amino acid sequence of SEQ ID NO: 14.
74. The anti-LAYN antibody, or an antigen-binding fragment thereof, according to item 69, wherein the antibody or the antigen-binding fragment comprises a heavy chain variable region comprising the amino acid sequence of SEQ ID NO: 13; and a light chain variable region comprising the amino acid sequence of SEQ ID NO: 14.
75. The anti-LAYN antibody, or an antigen-binding fragment thereof, according to any one of items 1 to 61 and 69 to 74, wherein the antibody or the antigen-binding fragment comprises (or consists of) a heavy chain comprising the amino acid sequence of SEQ ID NO: 15; and a light chain comprising the amino acid sequence of SEQ ID NO: 16.
76. The anti-LAYN antibody, or an antigen-binding fragment thereof, according to any one of items 1 to 61, wherein the antibody or the antigen-binding fragment comprises:
   a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 142; and
   a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6.
77. The anti-LAYN antibody, or an antigen-binding fragment thereof, according to item 76, wherein the antibody or the antigen-binding fragment comprises a heavy chain variable region comprising an amino acid sequence at least 80% identical to the amino acid sequence of SEQ ID NO: 143; and a light chain variable region comprising an amino acid sequence at least 80% identical to the amino acid sequence of SEQ ID NO: 79.
78. The anti-LAYN antibody, or an antigen-binding fragment thereof, according to item 76, wherein the antibody or the antigen-binding fragment comprises a heavy chain variable region comprising an amino acid sequence at least 85% identical to the amino acid sequence of SEQ ID NO: 143; and a light chain variable region comprising an amino acid sequence at least 85% identical to the amino acid sequence of SEQ ID NO: 79.
79. The anti-LAYN antibody, or an antigen-binding fragment thereof, according to item 76, wherein the antibody or the antigen-binding fragment comprises a heavy chain variable region comprising an amino acid sequence at least 90% identical to the amino acid sequence of SEQ ID NO: 143; and a light chain variable region comprising an amino acid sequence at least 90% identical to the amino acid sequence of SEQ ID NO: 79.
80. The anti-LAYN antibody, or an antigen-binding fragment thereof, according to item 76, wherein the antibody or the antigen-binding fragment comprises a heavy chain variable region comprising an amino acid sequence at least 95% identical to the amino acid sequence of SEQ ID NO: 143; and a light chain variable region comprising an amino acid sequence at least 95% identical to the amino acid sequence of SEQ ID NO: 79.
81. The anti-LAYN antibody, or an antigen-binding fragment thereof, according to item 76, wherein the antibody or the antigen-binding fragment comprises a heavy chain variable region comprising the amino acid sequence of SEQ ID NO: 143; and a light chain variable region comprising the amino acid sequence of SEQ ID NO: 79.
82. The anti-LAYN antibody, or an antigen-binding fragment thereof, according to any one of items 1 to 61 and 76 to 81, wherein the antibody or the antigen-binding fragment comprises (or consists of) a heavy chain comprising the amino acid sequence of SEQ ID NO: 144; and a light chain comprising the amino acid sequence of SEQ ID NO: 81.
83. The anti-LAYN antibody, or an antigen-binding fragment thereof, according to any one of items 1 to 61, wherein the antibody or the antigen-binding fragment comprises: a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6.
84. The anti-LAYN antibody, or an antigen-binding fragment thereof, according to item 83, wherein the antibody or the antigen-binding fragment comprises a heavy chain variable region comprising an amino acid sequence at least 80% identical to the amino acid sequence of SEQ ID NO: 34; and a light chain variable region comprising an amino acid sequence at least 80% identical to the amino acid sequence of SEQ ID NO: 79.
85. The anti-LAYN antibody, or an antigen-binding fragment thereof, according to item 83, wherein the antibody or the antigen-binding fragment comprises a heavy chain variable region comprising an amino acid sequence at least 85% identical to the amino acid sequence of SEQ ID NO: 34; and a light chain variable region comprising an amino acid sequence at least 85% identical to the amino acid sequence of SEQ ID NO: 79.
86. The anti-LAYN antibody, or an antigen-binding fragment thereof, according to item 83, wherein the antibody or the antigen-binding fragment comprises a heavy chain variable region comprising an amino acid sequence at least 90% identical to the amino acid sequence of SEQ ID NO: 34; and a light chain variable region comprising an amino acid sequence at least 90% identical to the amino acid sequence of SEQ ID NO: 79.
87. The anti-LAYN antibody, or an antigen-binding fragment thereof, according to item 83, wherein the antibody or the antigen-binding fragment comprises a heavy chain variable region comprising an amino acid sequence at least 95% identical to the amino acid sequence of SEQ ID NO: 34; and a light chain variable region comprising an amino acid sequence at least 95% identical to the amino acid sequence of SEQ ID NO: 79.
88. The anti-LAYN antibody, or an antigen-binding fragment thereof, according to item 83, wherein the antibody or the antigen-binding fragment comprises a heavy chain variable region comprising the amino acid sequence of SEQ ID NO: 34; and a light chain variable region comprising the amino acid sequence of SEQ ID NO: 79.
89. The anti-LAYN antibody, or an antigen-binding fragment thereof, according to any one of items 1 to 61 and 83 to 88, wherein the antibody or the antigen-binding fragment comprises (or consists of) a heavy chain comprising the amino acid sequence of SEQ ID NO: 36; and a light chain comprising the amino acid sequence of SEQ ID NO: 81.
90. The anti-LAYN antibody, or an antigen-binding fragment thereof, according to any one of the previous items, wherein the antibody or the antigen-binding fragment comprises a heavy chain constant region selected from IgG₁, IgG₂, IgG₃, IgG₄, IgM, IgA and IgE or a variant thereof.
91. The anti-LAYN antibody, or an antigen-binding fragment thereof, according to any one of the previous items, wherein the antibody or the antigen-binding fragment comprises an Fc region.
92. The anti-LAYN antibody or antigen binding fragment thereof according to any one of the previous items, wherein said antibody comprises an (human) IgG₁ heavy chain constant region.
93. The anti-LAYN antibody or antigen-binding fragment thereof according to any one of the previous items, wherein the anti-LAYN antibody or the antigen binding fragment comprises a heavy chain lacking a C-terminal lysine residue or a heavy chain including a C-terminal lysine residue.
94. The anti-LAYN antibody or antigen-binding fragment thereof according to any one of the previous items, wherein the anti-LAYN antibody or antigen-binding fragment thereof comprises a light chain constant region selected from the group consisting of a kappa and a lambda light chain.
95. The anti-LAYN antibody or antigen binding fragment thereof according to any one of the previous items, wherein said antibody comprises a heavy chain comprising an amino acid sequence at least 95% identical to the amino acid sequence of SEQ ID NO: 173; and a light chain comprising an amino acid sequence at least 95% identical to the amino acid sequence of SEQ ID NO: 174.
96. The anti-LAYN antibody or antigen-binding fragment thereof according to any one of the previous items, wherein the anti-LAYN antibody or antigen-binding fragment thereof comprises a heavy chain constant region wherein the heavy chain constant region is of a human IgG1 with (i) G236A/A330L/1332E or (ii) S239D/1332E substitutions according to the EU-index of Kabat.
97. The anti-LAYN antibody or antigen binding fragment thereof according to any one of the previous items, wherein said antibody comprises a heavy chain comprising the amino acid sequence of SEQ ID NO: 173; and a light chain comprising the amino acid sequence of SEQ ID NO: 174.
98. The anti-LAYN antibody or antigen-binding fragment thereof according to any one of the previous items, wherein the anti-LAYN antibody or the antigen binding fragment is afucosylated.
99. The anti-LAYN antibody or antigen-binding fragment thereof according to any one of the previous items, wherein the antibody is a monoclonal antibody.
100. The anti-LAYN antibody or antigen-binding fragment thereof according to any one of the previous items, wherein the anti-LAYN antibody or antigen-binding fragment thereof is a monospecific antibody.
101. The anti-LAYN antibody or antigen binding fragment thereof according to any one of the previous items, wherein the antibody is a human, humanized, or chimeric antibody, preferably a humanized antibody.
102. The anti-LAYN antibody or antigen-binding fragment thereof according to any one of the previous items, wherein the anti-LAYN antibody or antigen-binding fragment thereof binds the extracellular domain of human LAYN isoform 2 (such as SEQ ID NO: 190) at pH 7.4 with a binding affinity (K_{D}) of 50 nM or lower, 40 nM or lower, 30 nM or lower, 25 nM or lower, 20 nM or lower, 15 nM or lower, or 10 nM or lower.
103. The anti-LAYN antibody or antigen-binding fragment thereof according to any one of the previous items, wherein the anti-LAYN antibody or antigen-binding fragment thereof binds the extracellular domain of human LAYN isoform 2 (such as SEQ ID NO: 190) at pH 6.0 with a binding affinity (K_{D}) of 50 nM or lower, 40 nM or lower, 30 nM or lower, 25 nM or lower, 20 nM or lower.
104. An anti-LAYN antibody, or an antigen-binding fragment thereof, that specifically binds to human LAYN isoform 2, wherein the antibody or antigen-binding fragment competes for binding to LAYN with or binds the same LAYN epitope as the antibody according to any one of items 62 to 89.
105. A pharmaceutical composition comprising the antibody, or an antigen-binding fragment thereof, according to any one of the previous items and, optionally, a pharmaceutically acceptable carrier.
106. The anti-LAYN antibody, or an antigen-binding fragment thereof, according to any one of items 1-104 or the pharmaceutical composition of item 105 for use as a medicament.
107. The anti-LAYN antibody, or an antigen-binding fragment thereof, according to any one of items 1-104 or the pharmaceutical composition of item 105 for use in the treatment of cancer.
108. The anti-LAYN antibody, or an antigen-binding fragment thereof, or the pharmaceutical composition for use according to item 107, wherein the cancer is a solid tumor.
109. The anti-LAYN antibody, or an antigen-binding fragment thereof, or the pharmaceutical composition for use according to item 107, wherein the cancer is selected from the group consisting of non-small cell lung cancer (NSCLC), head and neck squamous cell carcinoma (HNSCC), colorectal cancer (CRC), ovarian cancer (OC), breast cancer, renal cell carcinoma (RCC), hepatocellular carcinoma (HCC), gastric cancer, bladder cancer, pancreatic cancer and melanoma.
110. The anti-LAYN antibody, or an antigen-binding fragment thereof, or the pharmaceutical composition for use according to any one of items 107-109, wherein the cancer is selected from the group consisting of colorectal cancer (CRC), non-small cell lung cancer (NSCLC), breast cancer, melanoma, hepatocellular carcinoma (HCC), and head and neck squamous cell carcinoma (HNSCC).
111. The anti-LAYN antibody, or an antigen-binding fragment thereof, or the pharmaceutical composition for use according to any one of items 107-110, wherein the anti-LAYN antibody, or the fragment thereof, or the pharmaceutical composition is administered in combination with an immune checkpoint inhibitor.
112. The anti-LAYN antibody, or an antigen-binding fragment thereof, or the pharmaceutical composition for use according to item 111, wherein the immune checkpoint inhibitor is a PD-1 antagonist.
113. The anti-LAYN antibody, or an antigen-binding fragment thereof, or the pharmaceutical composition for use according to any one of items 107-112, wherein the anti-LAYN antibody, or the fragment thereof, or the pharmaceutical composition is administered in combination with pembrolizumab.
114. The anti-LAYN antibody, or an antigen-binding fragment thereof, or the pharmaceutical composition for use according to any one of items 107-112, wherein the anti-LAYN antibody, or the fragment thereof, or the pharmaceutical composition is administered in combination with nivolumab.
115. The anti-LAYN antibody, or an antigen-binding fragment thereof, or the pharmaceutical composition for use according to any one of items 107-112, wherein the anti-LAYN antibody, or the fragment thereof, or the pharmaceutical composition is administered in combination with ezabenlimab.
116. The anti-LAYN antibody, or an antigen-binding fragment thereof, or the pharmaceutical composition for use according to any one of items 107-115, wherein the anti-LAYN antibody, or the fragment thereof, or the pharmaceutical composition is administered parenterally.
117. A method of treating or preventing or reducing the risk of occurrence of a cancer comprising administering an effective amount of the antibody, or an antigen-binding fragment thereof, according to any one of items 1-104 or of the pharmaceutical composition of item 105 to a patient in need thereof.
118. A method of treating cancer, or preventing cancer, or reducing the risk of occurrence of a cancer, in a patient, comprising administering a therapeutically effective amount of the antibody, or an antigen-binding fragment thereof, according to any one of items 1-104 or of the pharmaceutical composition of item 105 to the patient in need thereof.
119. The method according to item 117 or 118, wherein the cancer is a solid tumor.
120. The method according to item 117 or 118, wherein the cancer is selected from the group consisting of non-small cell lung cancer (NSCLC), head and neck squamous cell carcinoma (HNSCC), colorectal cancer (CRC), ovarian cancer (OC), breast cancer, renal cell carcinoma (RCC), hepatocellular carcinoma (HCC), gastric cancer, bladder cancer, pancreatic cancer and melanoma.
121. The method according to any one of items 117-120, wherein the cancer is selected from the group consisting of colorectal cancer (CRC), non-small cell lung cancer (NSCLC), breast cancer, melanoma, hepatocellular carcinoma (HCC), and head and neck squamous cell carcinoma (HNSCC).
122. The method according to any one of items 117-121, wherein the anti-LAYN antibody, or the fragment thereof, or the pharmaceutical composition is administered in combination with an immune checkpoint inhibitor.
123. The method according to item 122, wherein the immune checkpoint inhibitor is a PD-1 antagonist.
124. The method according to any one of items 117-123, wherein the anti-LAYN antibody, or the fragment thereof, or the pharmaceutical composition is administered in combination with pembrolizumab.
125. The method according to any one of items 117-123, wherein the anti-LAYN antibody, or the fragment thereof, or the pharmaceutical composition is administered in combination with nivolumab.
126. The method according to any one of items 117-123, wherein the anti-LAYN antibody, or the fragment thereof, or the pharmaceutical composition is administered in combination with ezabenlimab.
127. The method according to any one of items 117-126, wherein the anti-LAYN antibody, or the fragment thereof, or the pharmaceutical composition is administered parenterally.
128. Use of the antibody, or an antigen-binding fragment thereof, according to any one of items 1-104 or the pharmaceutical composition of item 105 for the manufacture of a medicament for the treatment of cancer.
129. The use according to item 128, wherein the cancer is a solid tumor.
130. The use according to item 128, wherein the cancer is selected from the group consisting of non-small cell lung cancer (NSCLC), head and neck squamous cell carcinoma (HNSCC), colorectal cancer (CRC), ovarian cancer (OC), breast cancer, renal cell carcinoma (RCC), hepatocellular carcinoma (HCC), gastric cancer, bladder cancer, pancreatic cancer and melanoma.
131. The use according to any one of items 128-130, wherein the cancer is selected from the group consisting of colorectal cancer (CRC), non-small cell lung cancer (NSCLC), breast cancer, melanoma, hepatocellular carcinoma (HCC), and head and neck squamous cell carcinoma (HNSCC).
132. The use according to any one of items 128-131, wherein the anti-LAYN antibody, or the fragment thereof, or the pharmaceutical composition is to be used in combination with an immune checkpoint inhibitor.
133. The use according to item 132, wherein the immune checkpoint inhibitor is a PD-1 antagonist.
134. The use according to any one of items 128-133, wherein the anti-LAYN antibody, or the fragment thereof, or the pharmaceutical composition is to be used in combination with pembrolizumab.
135. The use according to any one of items 128-133, wherein the anti-LAYN antibody, or the fragment thereof, or the pharmaceutical composition is to be used in combination with nivolumab.
136. The use according to any one of items 128-133, wherein the anti-LAYN antibody, or the fragment thereof, or the pharmaceutical composition is to be used in combination with ezabenlimab.
137. The use according to any one of items 128-136, wherein the anti-LAYN antibody, or the fragment thereof, or the pharmaceutical composition is administered parenterally.
138. A pharmaceutical composition comprising the antibody, or an antigen-binding fragment thereof, according to any one of items 1-104 and an immune checkpoint inhibitor.
139. The pharmaceutical composition according to item 138, wherein the immune checkpoint inhibitor is a PD-1 antagonist.
140. A kit comprising the antibody, or an antigen-binding fragment thereof, according to any one of items 1-104 and an immune checkpoint inhibitor.
141. The kit according to item 140, wherein the immune checkpoint inhibitor is a PD-1 antagonist.
142. The kit according to item 140 or 141, wherein the immune checkpoint inhibitor is pembrolizumab.
143. The kit according to item 140 or 141, wherein the immune checkpoint inhibitor is nivolumab.
144. The kit according to item 140 or 141, wherein the immune checkpoint inhibitor is ezabenlimab.
145. The kit according to any one of items 140 to 144, wherein the kit comprises a pharmaceutical composition comprising the anti-LAYN antibody, or an antigen-binding fragment thereof, and a pharmaceutical composition comprising the immune checkpoint inhibitor.
146. A polynucleotide encoding a sequence comprising the heavy chain variable region and/or the light chain variable region of the antibody, or an antigen-binding fragment thereof, according to any one of items 1-104.
147. A plurality of polynucleotides encoding a sequence comprising the heavy chain variable region and/or the light chain variable region of the antibody, or an antigen-binding fragment thereof, according to any one of items 1-104.
148. A plurality of polynucleotides comprising:
   (i) a first polynucleotide (comprising a nucleic acid sequence) encoding the heavy chain variable region or the heavy chain of an anti-LAYN antibody, or antigen-binding fragment thereof, according to any one of items 1-104; and
   (ii) a second polynucleotide (comprising a nucleic acid sequence) encoding the (corresponding) light chain variable region or the light chain of said anti-LAYN antibody, or antigen-binding fragment thereof, according to any one of items 1-104.
149. A vector comprising the polynucleotide according to item 146 or the plurality of polynucleotides according to item 147 or 148.
150. A plurality of vectors comprising the plurality of polynucleotides according to item 147 or 148.
151. The vector according to item 149 or the plurality of vectors according to item 150, wherein the vector or the plurality of vectors is/are expression vectors.
152. A host cell comprising the polynucleotide or the plurality of polynucleotides according to any one of items 146-148.
153. A host cell comprising the vector or the plurality of vectors according to any one of items 149-151.
154. The host cell according to item 152 or 153, wherein the host cell is a mammalian cell, a CHO cell and/or a Fut8-knockout cell.
155. A method for production of an anti-LAYN antibody, or an antigen-binding fragment thereof, comprising the steps:
   (a) Cultivating the host cell according to any one of items 152-154, preferably under conditions allowing the expression of the anti-LAYN antibody or antigen-binding fragment thereof; and
   (b) Recovering the anti-LAYN antibody or antigen-binding fragment thereof.
156. The method according to item 155 comprising a step of:
   (c) Purifying said anti-LAYN antibody or antigen-binding fragment thereof.
157. An (in-vitro) method of determining expression of LAYN using the anti-LAYN antibody, or an antigen-binding fragment thereof, according to any one of items 1-104.
158. A method of reducing or depleting intratumoral regulatory T cells, the method comprising the use of the anti-LAYN antibody, or an antigen-binding fragment thereof, according to any one of items 1-104.

### BRIEF DESCRIPTION OF THE FIGURES

A brief description of the appended figures will be given in the following. The figures are intended for illustration in more detail. However, they are not intended to limit the subject matter of the disclosure in any way.

FIG. 1 shows (A) an alignment of the extracellular domains (ECD) of human LAYN isoform 2 (LAYN-v2 ECD; SEQ ID NO: 191), human LAYN isoform 1 (LAYN-v1 ECD; SEQ ID NO: 197), human LAYN isoform 3 including a putative signal sequence (underligned) (LAYN-v3 ECD; SEQ ID NO: 198) and human LAYN isoform 4 including a putative signal sequence (underligned) (LAYN-v4 ECD; SEQ ID NO: 199) with boxes indicating sections of LAYN, which correspond to amino acids 52-67 and 123-138 of SEQ ID NO: 191 (LAYN-v2 ECD); and (B) an alignment of the extracellular domains (ECD) of human LAYN isoform 2 (“LAYN v2 ECD”; SEQ ID NO: 191), cynomolgus LAYN isoform 2 ortholog (“LAYN ECD (Macaca)”; SEQ ID NO: 200), dog LAYN isoform 2 ortholog (“LAYN ECD (Canis)”; SEQ ID NO: 201), rat LAYN isoform 2 ortholog (“LAYN ECD (Rattus)”; SEQ ID NO: 202), mouse LAYN isoform 2 ortholog (“LAYN ECD (Mus)”; SEQ ID NO: 203), rabbit LAYN isoform 2 ortholog (“LAYN ECD (Oryctolagus)”; SEQ ID NO: 204) and pig LAYN isoform 2 ortholog (“LAYN ECD (Sus)”; SEQ ID NO: 205), with boxes indicating sections of LAYN, which correspond to amino acids 52-67 and 123-138 of SEQ ID NO: 191 (LAYN-v2 ECD).

FIG. 2 shows an alignment of (A) VH and (B) VL sequences of exemplary antibodies of the present disclosure, which bind to LAYN with high binding affinity (K_{D} less than 50 nM, as described in Example 3), and the respective consensus sequences. Boxes indicate CDR1, CDR2 and CDR3 sequences according to different numbering schemes: Kabat (solid line), Aho (long-dashed line), CCG (dashed line), Chothia (dash-dotted line) and IMGT (dotted line).

FIG. 3 shows for Example 5 the binding of anti-LAYN antibodies to activated Treg expressing LAYN. In vitro expanded Tregs of healthy donors were activated with CD3/CD28 stimulation in the presence of IL-2 and incubated with serial dilutions of anti-LAYN antibodies. Bound anti-LAYN antibodies were detected with an Alexa-Fluor 647-labeled anti-human IgG (H+L) secondary antibody.

FIG. 4 shows for Example 6 CD16 crosslinking of anti-LAYN antibodies to CD16A reporter cells upon binding to LAYN-expressing Expi293 cells.

FIG. 5 shows for Example 6 ADCP reporter cell activity of anti-LAYN antibodies upon binding to LAYN-expressing Jurkat cells.

FIG. 6 shows for Example 10 CD16 crosslinking to CD16A reporter cells upon binding to LAYN-expressing Jurkat cells.

FIG. 7 shows for Example 11 ADCC induced by Fc variants of anti-LAYN antibodies in HEK cells expressing LAYN as target cells.

FIG. 8 shows for Example 12 tumor growth (A) and survival (B) in the MC38 tumor model in huLAYN mice after intraperitoneal treatment with a single indicated dose of anti-LAYN antibody moA85.

FIG. 9 shows for Example 12 MC38 tumor growth of individual huLAYN mice treated intraperitoneally with 10 mg/kg isotype control (A), 0.1 mg/kg anti-LAYN antibody moA85 (B), 1.0 mg/kg anti-LAYN antibody moA85 (C) or 10 mg/kg anti-LAYN antibody moA85 (D).

FIG. 10 shows for Example 12 tumor growth (A) and survival (B) in the MC38 tumor model in huLAYN animals after a single intravenous treatment with different doses of anti-LAYN antibody moA1 as indicated.

FIG. 11 shows for Example 12 MC38 tumor growth of individual huLAYN mice treated intravenously with 10 mg/kg isotype control (A), 0.1 mg/kg anti-LAYN antibody moA1 (B), 1.0 mg/kg anti-LAYN antibody moA1 (C) or 10 mg/kg anti-LAYN antibody moA1 (D).

FIG. 12 shows for Example 12 tumor growth (A) and survival (B) in the MC38 tumor model in huLAYN animals after intravenous treatment with a single dose of 10 mg/kg of anti-LAYN antibodies moA1 and moA2.

FIG. 13 shows for Example 12 MC38 tumor growth of individual huLAYN mice treated intravenously once with 10 mg/kg isotype control (A), 10 mg/kg anti-LAYN antibody moA1 (B) or 10 mg/kg anti-LAYN antibody moA2 (C).

FIG. 14 shows for Example 13 absolute numbers of tumor-infiltrating regulatory T cells (A) and tumor-infiltrating CD8+ T cells (B) and expression of activation markers Granzyme B and Ki67 in tumor-infiltrating CD8+ T cells (C) at different timepoints after the intraperitoneal administration of indicated doses of moA85 anti-LAYN antibody in MC38 tumor model in huLAYN animals.

FIG. 15 shows for Example 13 absolute numbers of tumor-infiltrating regulatory T cells (A) and tumor-infiltrating CD8+ T cells (B) at day 7 after the intravenous administration of indicated doses of moA1 anti-LAYN antibody in MC38 tumor model in huLAYN animals.

FIG. 16 shows for Example 13 absolute numbers of tumor-infiltrating regulatory T cells at day 7 after a single intravenous administration of 10 mg/kg anti-LAYN antibodies moA1 or moA2 in MC38 tumor-bearing huLAYN mice.

FIG. 17 shows for Example 14 tumor growth (A) and survival (B) in the MC38 tumor model in huLAYN mice after intraperitoneal treatment with anti-LAYN antibody moA85 and anti-PD1 mIgG1D265A antibody, either alone or in combination.

FIG. 18 shows for Example 14 MC38 tumor growth of individual huLAYN mice treated intraperitoneally with indicated dose of isotype (A), moA85 anti-LAYN antibody (B), anti-PD1 mIgG1D265A antibody (C) or the combination of anti-LAYN and anti-PD1 antibodies (D).

FIG. 19 shows for Example 14 tumor growth (A) and survival (B) in the MB49 tumor model in huLAYN animals after intraperitoneal treatment with anti-LAYN antibody moA85 and anti-PD1 mIgG1D265A antibody, either alone or in combination.

FIG. 20 shows for Example 14 MB49 tumor growth of individual huLAYN mice treated intraperitoneally with indicated dose of isotype (A), anti-PD1 mIgG1D265A antibody (B), moA85 anti-LAYN antibody (C) or the combination of anti-LAYN and anti-PD1 antibodies (D).

FIG. 21 shows for Example 14 tumor growth (A) and survival (B) in the MC38 tumor model in huLAYN mice after intraperitoneal treatment with anti-LAYN antibody moA1 and anti-PD1 mIgG1D265A antibody, either alone or in combination.

FIG. 22 shows for Example 14 MC38 tumor growth of individual huLAYN mice treated intraperitoneally with indicated dose of isotype controls (A), anti-PD1 mIgG1D265A antibody (B), moA1 anti-LAYN antibody (C) or the combination of anti-LAYN moA1 and anti-PD1 antibodies (D).

FIG. 23 shows for Example 14 tumor growth (A) and survival (B) in the MB49 tumor model in huLAYN animals after intraperitoneal treatment with anti-LAYN antibody moA1 and anti-PD1 mIgG1D265A antibody, either alone or in combination.

FIG. 24 shows for Example 14 MB49 tumor growth of individual huLAYN mice treated intraperitoneally with indicated dose of isotype controls (A), anti-PD1 mIgG1D265A antibody (B), moA1 anti-LAYN antibody (C) or the combination of anti-LAYN moA1 and anti-PD1 antibodies (D).

FIG. 25 shows for Example 15 the reduction of human regulatory T cells (A) isolated from human NSCLC tumor tissue after incubation with an anti-LAYN antibody and stimulated allogenic NK cells. (B) Correlation of Treg reduction and LAYN expression across the analyzed NSCLC samples.

FIG. 26 shows for Example 16 the cryo-EM structure of LAYN in complex with Fab A85.

FIG. 27 shows for Example 16 (A), (B) the interface of LAYN with Fab A85 and (C) the LAYN ECD sequence (LAYN-v2-no-tag; SEQ ID NO: 191) with highlighted residues being within a distance of no more than 4 Å of Fab A85.

FIG. 28 shows for Example 17 the BLI measurement showing no competition of (A) A1-A11 and A85_afuc2 with collagen IV binding to LAYN. In part (B) A86 and additional controls are shown as indicated, e.g., w/wo (with/without) CollagenIV and w/wo (with/without) LAYN protein. In part (C) no competition with CollagenIV was observed for the prior art antibodies: ML1Std (“Glasgow”), 4C11 and 3F7D7E2.

FIG. 29 shows for Example 18 thermal unfolding curves of moA85, A1, ML1Std and EPR11875 (A) and A86, P_huIgG1 and A85_afuc2 (B) measured with Prometheus Panta (Nanotemper) using the nanoDSF measurement method. Ratio (350 nm/330 nm) of intrinsic fluorescence from Trp and Tyr measured at 350 nm and 330 nm respectively, is plotted against the temperature gradient. The inflection temperature of the unfolding transition (inflection point) is depicted as dashed line. The start of the unfolding process (T onset) is depicted with a dotted line.

FIG. 30 shows for Example 19 flow cytometry of chondrolectin positive PC3 cells with anti-LAYN antibodies A86, A1 and A2, and anti-CHODL Ab as positive control. No binding of anti-LAYN Antibodies to chondrolectin on PC3 cells at 10 ug/ml was observed. (B) shows the same data as (A), but with separate curves (no overlay) for increased visibility of the three different conditions (secondary antibody only (2° only), isotype control (aTNP) and the respective anti-LAYN antibody (aLAYN)).

FIG. 31 shows for Example 20 binding of anti-LAYN antibody A86 to orthologs of LAYN v2 expressed in Expi293F cells in one representative experiment. Binding is depicted as cell frequency positive for Alexa Fluor 488 (A) or median fluorescence intensity (MFI) of Alexa Fluor 488 (B).

FIG. 32 shows for Example 21 competition of antibody A1 with other anti-LAYN antibodies: ML1Std, antibody 4C11 (Novus Biologicals), antibody MAB26371 (R&D 328002), 3F7D7E2 (Sino Biological), A2, A85_afuc2, A86, and P_huIgG1. (A) shows the complete experimental setup for antibodies ML1Std, 4C11, MAB26371 (R&D 328002), and 3F7D7E2. (B) focuses on the respective 2nd antibodies (as indicated) to evaluate their binding to the previously formed LAYN/A1 complex. (C) shows the complete experimental setup for antibodies A2, A85_afuc2, A86 and P_huIgG1. (D) focuses on the respective 2^{nd} antibodies (as indicated) to evaluate their binding to the previously formed LAYN/A1 complex. C1 and C2 are additional control experiments without (w/o) coupling of A1 or without (w/o) coupling of A1 and LAYN.

### DETAILED DESCRIPTION

This disclosure generally relates to anti-LAYN antibodies or antigen-binding fragments thereof. In some aspects, the anti-LAYN antibodies or antigen-binding fragments thereof are for diagnostic and/or therapeutic use, for example in a subject in need thereof, such as a human.

In the present description, numerous specific details are set forth to provide a full understanding of the subject technology. It will be apparent, however, to one of ordinary skill in the art that the subject technology may be practiced without some of these specific details. Accordingly, it is to be understood that the subject technology is not limited to the particular methodologies, protocols and reagents described herein. In other instances, well-known structures and techniques may have not been shown in detail so as not to obscure the subject technology. Preferences and options for a given aspect, feature, embodiment, or parameter should, unless the context indicates otherwise, be regarded as having been disclosed in combination with any and all preferences and options for all other aspects, features, embodiments and parameters of the invention.

### DEFINITIONS

Unless defined otherwise, all technical and scientific terms used herein have the same meanings as commonly understood by one of ordinary skill in the art in light of the disclosure and the context. As used in the specification, however, unless specified to the contrary, the following terms have the meaning indicated and the following conventions are adhered to.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present disclosure, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of the present disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the subject technology.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x plus or minus (±) 10%, for example, x±1%, or x±2%, or x±3%, or x±4%, or x±5%, or x±6%, or x±7%, or x±8%, or x±9%, or x±10%.

The terms, “antibody”, and “anti-LAYN antibody”, are used herein interchangeably and encompass monoclonal antibodies (including full length monoclonal antibodies), multispecific antibodies (e.g., bispecific antibodies), antibodies with minor modifications such as N- or C-terminal truncations and antibody fragments such as variable domains and other portions of antibodies that exhibit a desired biological activity, e.g., LAYN binding.

In general, antibodies can bind (by non-covalent interaction) to other molecules or structures known as “antigens”. This binding is usually “specific” in the sense that an antibody will only bind to a specific structure with high affinity. The unique part of the antigen recognized by an antibody is called an “epitope”, or antigenic determinant. The part of the antibody binding to the epitope is sometimes called “paratope” (also referred to as “antigen-binding site”) and resides in the variable domain(s), or variable region(s) (Fv) of the antibody, more specifically in the so-called complementary-determining region (CDRs). Typically, each variable region (i.e., heavy chain variable region (VH) and light chain variable region (VL)) comprises three CDRs, which are spaced apart by framework regions (FRs). As used herein, the heavy chain variable region (of an antibody or antigen-binding fragment thereof) is also referred to as “VH” (or “V_{H}”) and the light chain variable region (of an antibody or antigen-binding fragment thereof) is also referred to as “VL” (or “VL”). The (full-length) heavy chain (of an antibody or antigen-binding fragment thereof) is also referred to herein as “HC” and the (full-length) light chain (of an antibody or antigen-binding fragment thereof) is also referred to herein as “LC”. As used herein, a heavy chain CDR (a CDR in the heavy chain variable region) is also referred to as “HCDR”. Accordingly, the three CDRs of the heavy chain are referred to “HCDR1”, “HCDR2” and “HCDR3” (from N- to C-terminal side of the VH). As used herein, a light chain CDR (a CDR in the light chain variable region) is also referred to as “LCDR”. Accordingly, the three CDRs of the light chain are referred to “LCDR1”, “LCDR2” and “LCDR3” (from N- to C-terminal side of the VL).

The art has further developed antibodies and made them versatile tools in medicine and technology. Thus, in the context of the present disclosure the terms “antibody molecule” or “antibody” (used synonymously herein) do not only include antibodies as they may be found in nature, comprising e.g. two light chains and two heavy chains, or just two heavy chains as in camelid species. Instead, the term “antibody” encompasses all molecules comprising at least one paratope having (i) binding specificity to an antigen and (ii) preferably also structural similarity to a variable domain of an immunoglobulin.

Thus, an antibody, as defined herein, may be a polyclonal antibody, a monoclonal antibody, a human antibody, a humanized antibody, a chimeric antibody, a fragment of an antibody, such as a Fv, Fab, Fab′, or F(ab′)₂ fragment, a single chain antibody, in particular a single chain variable fragment (scFv), a Small Modular Immunopharmaceutical (SMIP), a domain antibody, a nanobody, a diabody.

The term “polyclonal” antibodies refers to a plurality/population of antibody molecules with different amino acid sequences, which are often directed to (binding to) the same antigen. They may be directed to the same, different or overlapping epitopes of the antigen. Polyclonal antibodies may be obtained from the blood of vertebrates after immunization with the antigen by processes well-known in the art.

The term “monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation that may be present. Monoclonal antibodies typically bind one antigenic epitope. A bispecific monoclonal antibody binds two distinct antigenic epitopes. A monoclonal antibody may be monospecific or multispecific such as bispecific, monovalent, bivalent or multivalent. They may be produced by any technique or methodology known in the art; including e.g., the hybridoma technology (Kohler G, Milstein C. Continuous cultures of fused cells secreting antibody of predefined specificity. Nature 1975; 256:495-7), or recombinant DNA methods known in the art (see, e.g., U.S. Pat. No. 4,816,567), or methods of isolation of monoclonal recombinantly produced using phage antibody libraries, using techniques described in Clackson et al., 1991, Nature 352:624-628, and Marks et al., 1991, J. Mol. Biol. 222:581-597; or recombinant expression in host cells (Norderhaug L, Olafsen T, Michaelsen T E, Sandlie I. (May 1997). “Versatile vectors for transient and stable expression of recombinant antibody molecules in mammalian cells.”. J Immunol Methods 204 (1): 77-87).

For application in humans, it is often desirable to reduce immunogenicity of antibodies originally derived from other species, like mouse. This may be achieved by “chimeric” antibodies or “humanized” antibodies.

As used herein, a “chimeric antibody” is understood to be an antibody comprising a sequence part (e.g. a variable domain) derived from one species (e.g. mouse) fused to a sequence part (e.g. the constant domains) derived from a different species (e.g. human). In particular, chimeric antibodies may comprise the heavy and light chain variable regions of an antibody from one species (e.g., a non-human mammal such as a mouse) and the heavy and light chain constant regions of another species (e.g., human) antibody. Chimeric antibodies can be obtained by linking the DNA sequences encoding the variable regions of the antibody from the first species (e.g., mouse) to the DNA sequences for the constant regions of the antibody from the second (e.g. human) species, for example by cloning of the DNA sequences encoding the variable regions of the antibody from the first species (e.g., mouse) into a (expression) vector, which may already contain the DNA sequences for the constant regions of the antibody from the second (e.g. human) species. The chimeric antibody may then be expressed by transforming a host with an expression vector containing the linked sequences to allow it to produce a chimeric antibody. Alternatively, the chimeric antibody also could be one in which one or more regions or domains of the heavy and/or light chain is identical with, homologous to, or a variant of the corresponding sequence in a monoclonal antibody from another immunoglobulin class or isotype, or from a consensus or germline sequence. Chimeric antibodies can include fragments of such antibodies, provided that the antibody fragment exhibits the desired biological activity of its parent antibody, for example binding to the same antigen or epitope (see, e.g., U.S. Pat. No. 4,816,567; and Morrison et al., 1984, Proc. Natl. Acad. Sci. USA 81:6851-6855). Methods of chimerization of antibodies are well-known in the art (e.g., Billetta R, Lobuglio A F. “Chimeric antibodies”. Int Rev Immunol. 1993; 10(2-3):165-76).

A “humanized antibody” is an antibody comprising a variable domain originally derived from a non-human species, wherein certain amino acids have been mutated to make the overall sequence of that variable domain more closely resemble to a sequence of a human variable domain. To maintain the binding of the original non-human antibody, mutations are often mostly introduced into the FRs rather than in the CDRs. Accordingly, a humanized antibody may comprise one or more FRs having substantially the amino acid sequence of a human immunoglobulin and one or more CDRs having substantially the amino acid sequence of a non-human immunoglobulin. This non-human amino acid sequence, often referred to as an “import” sequence, is typically taken from an “import” antibody domain, particularly a variable domain. Methods of humanization of antibodies are well-known in the art and described, for example, in Almagro et al., (2008) Frontiers in Bioscience 13, 1619-1633; in WO12092374 A2; or in Riechmann L, Clark M, Waldmann H, Winter G (1988). “Reshaping human antibodies for therapy”. Nature: 332:323.

The term “human antibody” as used herein includes antibodies or fragments thereof derived from human germline immunoglobulin sequences. In particular, the term “human antibody” includes antibodies containing somatic mutations, in particular in the variable regions, as compared to the germline sequence, such as antibodies found in (isolated) human (blood) samples. While a “human antibody” typically contains human constant regions, one or more of the constant regions, in particular in the Fc region, may be engineered as known in the art. The term “human antibody” is not intended to include antibodies in which CDR sequences derived from the germline of another (mammalian) species, such as a mouse, rat or rabbit, have been grafted onto human framework sequences. Thus, as used herein, the term “human antibody” refers to an antibody or fragment thereof in which every part of the protein (e.g., CDR, framework, constant regions (e.g., CH1, CH2, CH3, hinge, CL) and variable regions (VL, VH)) is substantially non-immunogenic in humans, with only minor sequence changes or variations as further described herein below. Thus, a human antibody is distinct from e.g., a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Technologies for creating such a “human antibody” are known in the art and include, without being limiting, phage display or use of transgenic animals (www. Ablexis.com/technology-alivamab.php; WO 90/05144; D. Marks, H. R. Hoogenboom, T. P. Bonnert, J. McCafferty, A. D. Griffiths and G. Winter (1991) “By-passing immunisation. Human antibodies from V-gene libraries displayed on phage.” J. Mol. Biol., 222, 581-597; Knappik et al., J. Mol. Biol. 296:57-86, 2000; S. Carmen and L. Jermutus, “Concepts in antibody phage display”. Briefings in Functional Genomics and Proteomics 2002 1(2): 189-203; Lonberg N, Huszar D. “Human antibodies from transgenic mice”. Int Rev Immunol. 1995; 13(1):65-93.; Brüggemann M, Taussig M J. “Production of human antibody repertoires in transgenic mice”. Curr Opin Biotechnol. 1997 August; 8(4):455-8.).

The chimeric, humanized or human antibodies or antigen-binding fragments thereof of the present disclosure may further be engineered. Such engineering includes without limitation the removal or exchange of undesired amino acids, for example to reduce immunogenicity in humans, or to avoid deamidation, undesirable charges or lipophilicity or non-specific binding. Such removal or exchange of undesired amino acids can, for example, be introduced by random or site-specific mutagenesis in vitro or by somatic mutation in vivo. Moreover, in connection with chimeric or humanized antibodies, it will be understood that certain mouse FR residues may be retained in an antibody or fragment thereof.

The terms “antibody fragment”, “antigen-binding fragment”, “anti-LAYN antibody fragment”, “engineered anti-LAYN antibody fragment” refer to a portion of a full length (anti-LAYN) antibody, in which a variable region or a functional capability is retained, for example, LAYN binding. A number of technologies have been developed for placing variable domains of immunoglobulins, or molecules derived from such variable domains, in a different molecular context. Antibody fragments may be smaller in size compared to (full-length) immunoglobulins, and may comprise a single amino acid chain or several amino acid chains. Examples of antibody fragments include, but are not limited to, a Fab, Fab′, F(ab′)₂, Fd, Fv, scFv and scFv-Fc fragment, a diabody, a linear antibody, a single-chain antibody, a minibody, a diabody formed from antibody fragments, and multispecific antibodies formed from antibody fragments.

Antibody fragments may be obtained by fragmentation of immunoglobulins, e.g. by proteolytic digestion, or by recombinant expression of such fragments. For example, full length antibodies may be treated with enzymes such as papain or pepsin to generate useful antibody fragments.

Papain digestion is used to produce two identical antigen-binding antibody fragments called “Fab” fragments, each with a single antigen-binding site, and a residual “Fc” fragment. The Fab fragment also contains the constant domain of the light chain (CL) and the CH1 domain of the heavy chain. Accordingly, in Fab molecules, the variable domains are each fused to an immunoglobulin constant domain, preferably of human origin. Thus, the heavy chain variable domain may be fused to a CH1 domain (a so-called Fd fragment), and the light chain variable domain may be fused to a CL domain. Fab molecules may also be produced by recombinant expression of respective nucleic acids in host cells.

Another example of antibody fragments according to the present disclosure are Fab′ fragments. Fab′ fragments differ from Fab fragments by the presence of additional residues including one or more cysteines from the antibody hinge region at the C-terminus of the CH1 domain.

Pepsin treatment yields a F(ab′)₂ fragment that has two antigen-binding sites and is still capable of cross-linking antigen. F(ab′)₂ antibody fragments are pairs of Fab′ fragments linked by cysteine residues in the hinge region. Other chemical couplings of antibody fragments are also known.

A “Fv” fragment contains a complete antigen-recognition and binding site consisting of a dimer of one heavy and one light chain variable domain in tight, non-covalent association. In this configuration, the three CDRs of each variable domain interact to define an antigen-biding site on the surface of the VH-VL dimer. Collectively, the six CDRs confer antigen-binding specificity to the antibody.

Antibody fragments may also include “single-chain Fv” or “scFv” fragments. A “single-chain Fv” or “scFv” antibody fragment is a single chain Fv variant comprising the VH and VL domains of an antibody where the domains are present in a single polypeptide chain. The single chain Fv is capable of recognizing and binding antigen. The scFv polypeptide may optionally also contain a polypeptide linker positioned between the VH and VL domains in order to facilitate formation of a desired three-dimensional structure for antigen binding by the scFv (see, e.g., Plueckthun, 1994, In The Pharmacology of monoclonal Antibodies, Vol. 113, Rosenburg and Moore eds., Springer-Verlag, New York, pp. 269-315). Examples of linkers include short linkers comprising serine(S) and/or glycine (G) residues, which are known in the art and described, e.g., in WO 88/01649; WO 91/17271; Huston et al; International Reviews of Immunology, Volume 10, 1993, 195-217.

Antibody fragments may also form tandem Fd segments, which comprise a pair of tandem Fd segments (V_{H}-C_{H}1-V_{H}-C_{H}1) to form a pair of antigen binding regions. These “linear antibodies” can be bispecific or monospecific as described in, for example, Zapata et al. 1995, Protein Eng. 8(10):1057-1062.

“Single domain antibodies” or “nanobodies” harbor an antigen-binding site in a single Ig-like domain (WO 94/04678; WO 03/050531, Ward et al., Nature. 1989 Oct. 12; 341(6242):544-6; Revets et al., Expert Opin Biol Ther. 5(1):111-24, 2005). One or more single domain antibodies with binding specificity for the same or a different antigen may be linked together. Diabodies are bivalent antibody molecules consisting of two amino acid chains comprising two variable domains (WO 94/13804, Holliger et al., Proc Natl Acad Sci USA. 1993 Jul. 15; 90(14):6444-8). Other examples of antibody-like molecules are immunoglobulin super family antibodies (IgSF; Srinivasan and Roeske, Current Protein Pept. Sci. 2005, 6(2): 185-96). A different concept leads to the so-called Small Modular Immunopharmaceutical (SMIP) which comprises a Fv domain linked to single-chain hinge and effector domains devoid of the constant domain CH1 (WO 02/056910).

As used herein, an antibody may comprise substantially all of at least one, and typically two, variable domains (such as contained, for example, in Fab, Fab′, F(ab′)₂, scFV, and Fv fragments). In some aspects, the antibody also includes at least a portion of an immunoglobulin Fc region, typically that of a human immunoglobulin. Ordinarily, the antibody will contain at least both the light chain variable region as well as at least the variable domain of a heavy chain. The antibody also may include one or more of the CH1, hinge, CH2, CH3, and/or CH4 regions of the heavy chain and/or the CL of the light chain, as appropriate.

In general, an antibody can be selected from any class of immunoglobulins, including IgM, IgG, IgD, IgA and IgE, and any isotype, including IgG₁, IgG₂, IgG₃, IgG₄, IgA₁ and IgA₂. An antibody can comprise sequences from more than one immunoglobulin class or isotype. In general, selecting particular modified or unmodified constant domains to optimize desired effector functions is within the ordinary skill in the art.

For example, the Fc region of an antibody mediates its serum half-life and effector functions, such as complement-dependent cytotoxicity (CDC), antibody-dependent cellular cytotoxicity (ADCC) and antibody-dependent cell phagocytosis (ADCP). Fc engineering can be employed to optimize antibody properties suited to the pharmacology activity required of them. Where such cytotoxic activity is not desirable, the constant domain may be of an isotype with reduced effector function, such as IgG₄, and/or may be modified with known modifications that reduce effector function. Where such cytotoxic activity is desirable, such as for destruction of a targeted cell, the constant domain may be of isotype with increased effector function and/or be modified with known modifications to increase effector function. Several mutations are known to either reduce or increase effector function. See, e.g., “The future of antibodies as cancer drugs” Janice M Reichert, Eugen Dhimolea, Drug Discov Today (2012) September; 17(17-18):954-63; PMID: 22561895, “Antibody Drug Discovery” (Volume 4 of Molecular medicine and medicinal chemistry) Clive R. Wood, World Scientific, 2012 ISBN 1848166281, 9781848166288; “FcγR requirements leading to successful immunotherapy” Immunol Rev. (2015) November; 268(1):104-22; PMID: 26497516.

The FRs and CDRs, or HVLs, of an engineered antibody, or antigen-binding fragment thereof, need not correspond precisely to the parental sequences, i.e., the sequences of the antibody as isolated prior to engineering. For example, a parental sequence may be altered (e.g., mutagenized) by substitution, insertion or deletion such that the resulting amino acid residue is no longer identical to the original residue in the corresponding position in either parental sequence. Typically, the antibody nevertheless retains a desired function, e.g. the function of binding to the targeted antigen, such as LAYN. Such alteration typically will not be extensive. Such alterations typically will be conservative alterations. Usually, at least 75% of the engineered antibody residues will correspond to those of the parental sequences, often at least 80%, more often at least 85%, even more often at least 90%, still more often at least 95%, and most frequently at least 98% or at least 99%.

Immunoglobulin residues that affect the interface between heavy and light chain variable regions (“the VL-VH interface”) are those that affect the proximity or orientation of the two chains with respect to one another. Certain residues that may be involved in interchain interactions include VL residues 34, 36, 38, 44, 46, 87, 89, 91, 96, and 98 and VH residues 35, 37, 39, 45, 47, 91, 93, 95, 100, and 103 (utilizing the numbering system set forth in Kabat et al., Sequences of Proteins of Immunological Interest, Vol. 1, 5th ed., 1991, Bethesda, MD: U.S. Dept. of Health and Human Services, Public Health Service, National Institutes of Health, NIH Publication No. 91-3242); U.S. Pat. No. 6,407,213 also discusses that residues such as VL residues 43 and 85, and VH residues 43 and 60 also may be involved in this interaction. While these residues are indicated for human IgG only, they are applicable across species.

The terms “consensus sequence” and “consensus antibody” refer to an amino acid (or nucleotide) sequence which comprises the most frequently occurring amino acid residue (or nucleotide) at each location in all immunoglobulins of any particular class, isotype, or subunit structure, e.g., a human immunoglobulin variable domain or a CDR. The consensus sequence may be based on immunoglobulins of a particular species or of many species. Thus, a consensus sequence is typically an amino acid (or nucleotide) sequence having at each position an amino acid residue (or nucleotide) that is present in one or more known immunoglobulins, but which may not exactly duplicate the entire amino acid (or nucleotide) sequence of any single immunoglobulin. The variable region consensus sequence may not be obtained from any naturally produced antibody or immunoglobulin. For example, the FRs of heavy and light chain (variable region) consensus sequences, and variants thereof, provide useful sequences for the preparation of human or humanized anti-LAYN antibodies. See, for example, U.S. Pat. Nos. 6,037,454 and 6,054,297.

In general, the antibodies, or antigen-binding fragments, of the present disclosure may be “isolated” antibodies, or antigen-binding fragments thereof. An “isolated” antibody is one that has been identified and separated and/or recovered from a component of its natural environment or from a cell culture from which it was expressed. An isolated antibody or antibody fragment may have one or more co- or post-translational modifications that arise during production, purification, and/or storage of the antibody or antibody fragment. Contaminant components of the antibody's natural environment are those materials that may interfere with diagnostic or therapeutic uses of the antibody, and can be enzymes, hormones, or other proteinaceous or non-proteinaceous solutes. An isolated antibody includes an antibody in situ within recombinant cells in which it is produced, since at least one component of the antibody's natural environment will not be present. Ordinarily however, an isolated antibody will be prepared by at least one purification step in which the recombinant cellular material is removed. In some aspects, the antibody will be purified to at least greater than 95% isolation by weight of antibody, for example purified to at least greater than 95%, 96%, 97%, 98%, or 99%.

Antibodies may be monospecific, bispecific or multispecific. “Monospecific” refers to a protein, such as an antibody, that specifically binds to a (single) specific epitope of an antigen. This may be achieved by an antibody having a single antigen-binding site (paratope), which is also referred to as “monovalent”. Examples thereof include, but are not limited to, Fv and scFv. Preferably, (monospecific) antibodies contain two identical paratopes (referred to as “bivalent”), such as in classical IgG-type antibodies. The monospecific antibodies provided herein, in particular the antigen-binding sites (paratopes) thereof, may be used to engineer bispecific or multispecific antibodies, which are also encompassed within the scope of the present disclosure. “Bispecific” refers to a protein, such as an antibody, that specifically binds two distinct antigens or two distinct epitopes within the same antigen. “Multispecific” refers to a protein, such as an antibody, that specifically binds two or more distinct antigens or two or more distinct epitopes within the same antigen. Accordingly, the term “multispecific” includes “bispecific”. Full length bispecific antibodies may be generated for example using Fab arm exchange (e.g., half-molecule exchange, exchanging one heavy chain-light chain pair) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond. Bispecific antibodies may also be generated, for example, using designs such as the Triomab/Quadroma (Trion Pharma/Fresenius Biotech), Knob-in-Hole (Genentech), CrossMAbs (Roche) and the electrostatically-induced CH3 interaction (Chugai, Amgen, NovoNordisk, Oncomed), the LUZ-Y (Genentech), the Strand Exchange Engineered Domain body (SEEDbody) (EMD Serono), the Biclonic (Merus) or as DuoBody® Products (Genmab A/S).

The antibody molecule may be fused (as a fusion protein) or otherwise linked (by covalent or non-covalent bonds) to other molecular entities having a desired impact on the properties of the antibody molecule. For example, it may be desirable to improve pharmacokinetic properties of antibody molecules, stability e.g. in body fluids such as blood, in particular in the case of single chain antibodies or domain antibodies. A number of technologies have been developed in this regard, in particular to prolong the half-life of such antibody molecules in the circulation, such as pegylation (WO 98/25971; WO 98/48837; WO 2004081026), fusing or otherwise covalently attaching the antibody molecule to another antibody molecule having affinity to a serum protein like albumin (WO 2004041865; WO 2004003019), or expression of the antibody molecule as fusion protein with all or part of a serum protein like albumin or transferrin (WO 01/79258).

The antibodies or antigen-binding fragment thereof may be conjugated to a label, either a label alone or a label and an additional second agent (prodrug, chemotherapeutic agent and the like). A labelled antibody, or antigen binding fragment, can be used, e.g., for diagnostic as well as therapeutic monitoring purposes. A label, as distinguished from the other second agents refers to an agent that is a detectable compound or composition and it may be conjugated directly or indirectly to an anti-LAYN antibody or antigen-binding fragment thereof of the present invention. The label may itself be detectable (e.g., radioisotope labels or fluorescent labels) or, in the case of an enzymatic label, may catalyze chemical alteration of a substrate compound or composition that is detectable. Labeled anti-LAYN antibodies or antigen-binding fragments thereof can be prepared and used in various applications including in vitro and in vivo diagnostics.

When referring to a ligand/receptor, antibody/antigen, or other binding pair, “specifically” or “selectively” indicates a binding reaction which is determinative of the presence of the protein in a heterogeneous population of proteins and other biologics. Thus, under designated conditions, a specified ligand binds to a particular receptor and does not bind in a significant amount to other proteins present in the sample. The terms “specifically binds” or “specific binding” in the context of a binding agent, e.g., an antibody or antigen-binding fragment thereof, refers to a binding agent that associates more frequently, more rapidly, with greater duration, with greater affinity, with greater avidity or with some combination of the above, to an antigen or an epitope within the antigen than with an unrelated antigen. In certain embodiments, an antibody or antigen-binding fragment thereof specifically binds to an antigen or epitope within an antigen with a K_{D} of about 0.1 mM or less, preferably less than about 1 μM. Because of the sequence identity between homologous proteins in different species, or variants of a protein within a single species, specific binding can include an antibody or antigen-binding fragment thereof that recognizes a protein in more than one species. It is understood that, in certain embodiments, an antibody or antigen-binding fragment thereof that specifically binds a first protein may or may not specifically bind a second protein. As such, “specific binding” does not necessarily require (although it can include) exclusive binding, i.e. binding to a single protein. Thus, an antibody or antigen-binding fragment thereof may, in certain embodiments, specifically bind more than one protein.

In some aspects, the antibody, or antigen-binding fragment thereof, binds to its antigen (e.g., LAYN) with an affinity that is at least two fold greater, preferably at least ten times greater, more preferably at least 20-times greater, and most preferably at least 100-times greater than the affinity with unrelated antigens.

Affinity is the interaction between a single antigen-binding site on an antibody molecule and a single epitope. It can be expressed by the association constant K_{A}=Kₐₛₛ/K_{diss}, or the dissociation constant K_{D}=K_{diss}/Kₐₛₛ.

In some aspects, the antibody binds to LAYN, such as human LAYN, in particular (the ECD of) human LAYN isoform 2 (e.g., having an amino acid sequence of SEQ ID NO: 190 or 191), with an affinity, as determined by surface plasmon resonance analysis (SPR; Malmqvist M., “Surface plasmon resonance for detection and measurement of antibody-antigen affinity and kinetics.”, Curr Opin Immunol. 1993 April; 5(2):282-6.). A specific exemplified method for performing SPR is described in Example 3. Preferably, the K_{D} value obtained by SPR for binding of the antibody to the antigen ranges from 1 pM to 100 μM, more preferably 1 pM to 1 μM. In some embodiments, the binding affinity is characterized by a K_{D} of about 1×10⁻⁷ M (100 nM) or less. In some embodiments, the binding affinity is characterized by a K_{D} of about 5×10⁻⁸ M (50 nM) or less.

The binding affinity of an antibody molecule may be enhanced by a process known as affinity maturation (Marks et al., 1992, Biotechnology 10:779-783; Barbas, et al., 1994, Proc. Nat. Acad. Sci, USA 91:3809-3813; Shier et al., 1996, Gene 169:147-155). Affinity matured antibodies are therefore also embraced in the present disclosure.

The terms “polynucleotide”, “nucleic acid” and “nucleic acid molecule” are used interchangeably herein. These terms include both, DNA and RNA molecules. In general, the polynucleotides/nucleic acids of the present disclosure may be “isolated”. An “isolated” polynucleotide is one that has been identified and separated and/or recovered from a component of its natural environment or from a cell including said nucleic acid, or that has been produced without a cell or its natural environment. An isolated nucleic acid/polynucleotide may be prepared by recombinant means (e.g., cloning or engineering), in particular without involvement of cells, or it may be purified from a cell.

As described herein, the present invention includes (sequence) variants, e.g. of the anti-LAYN antibodies or antigen-binding fragments thereof. In general, a (sequence) variant may refer to a nucleic acid sequence or to an amino acid sequence. It is understood that due to the redundancy of the genetic code variations in a nucleic acid sequence not always result in changes in the respective amino acid sequence (e.g., “silent” mutations). However, such silent mutations in nucleic acid sequences may be useful to optimize a nucleic acid sequence for expression, e.g., by codon optimization and other methods known in the art. On the other hand, a (sequence) variant of an anti-LAYN antibody or antigen binding fragment refers to a variant amino acid sequence, wherein at least one (e.g., 1, 2, 3, 4, 5, 6, 7, 8, 9, 10 or more) amino acid residue in the anti-LAYN antibody or antigen-binding fragment thereof are substituted, added or deleted. A (sequence) variant of an anti-LAYN antibody or antigen binding fragment typically refers to a reference sequence. In the context of the present specification, reference sequences (for variant antibodies) are usually the sequences of the exemplary antibodies P and A1-A104, preferably P and A1-A97, more preferably A1-A88, A90 and A91 as described herein (e.g., in Tables 2-8). A variant antibody may include mutations in any part of the sequence, i.e. (i) in the variable regions VH and/or VL, and/or (ii) in the constant regions (e.g., Fc engineering). However, it is preferred that the amino acid sequences of the specific sets of the six CDRs of the exemplary antibodies P and A1-A104, preferably P and A1-A97, more preferably A1-A88, A90 and A91, as described herein, are maintained (without changes). Accordingly, mutations, such as amino acid substitutions, are preferably introduced into framework region(s) and/or constant region(s) of the anti-LAYN antibody or antigen-binding fragment thereof (in particular exemplary antibodies P and A1-A104, preferably P and A1-A97, more preferably A1-A88, A90 and A91).

In some embodiments, variant anti-LAYN antibodies, or antigen-binding fragments thereof, have at least one (e.g., 1, 2, 3, 4, 5, 6, 7, 8, 9, 10 or more) amino acid residue(s) in the anti-LAYN antibody or antigen-binding fragment thereof substituted. “Substitution” typically refers to an amino acid residue of the reference sequence that has been removed and a different residue inserted in its place. The sites of greatest interest for substitutional mutagenesis in the variable regions (VH and/or VL) of an antibody typically include the framework regions. In general, for any mutation described herein for the variant anti-LAYN antibodies, or antigen-binding fragments thereof, conservative substitutions are preferred (unless specifically indicated otherwise). Non-conservative substitutions are also possible, but less preferred.

Exemplary conservative substitutions are shown in Table 1 under the heading of “preferred substitutions”. If such substitutions result in a change in biological activity, then more substantial changes, denominated “exemplary substitutions”, or as further described below in reference to amino acid classes, may be introduced and the products screened.

TABLE 1
Exemplary amino acid substitutions

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | arg; asn; gln; lys; | arg |
| lle (I) | leu; val; met; ala; phe; norleucine | leu |
| Leu (L) | ile; norleucine; val; met; ala; phe | ile |
| Lys (K) | arg; gln; asn | arg |
| Met (M) | leu; phe; ile | leu |
| Phe (F) | tyr; leu; val; ile; ala; | tyr |
| Pro (P) | ala | ala |
| Ser (S) | thr | thr |
| Thr (T) | ser | ser |
| Trp (W) | tyr; phe | tyr |
| Tyr (Y) | phe;trp; thr; ser | phe |
| Val (V) | leu; ile; met; phe ala; norleucine; | leu |

Naturally occurring amino acid residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Conservative substitutions will entail exchanging a member of one of these classes for another member of the same class. Non-conservative substitutions will entail exchanging a member of one of these classes for another class.

Any cysteine residue not involved in maintaining the proper conformation of the anti-LAYN antibody or antigen-binding fragment thereof also may be substituted, generally with serine, to improve the oxidative stability of the molecule, prevent aberrant crosslinking, or provide for established points of conjugation to a cytotoxic or cytostatic compound. Conversely, cysteine bond(s) may be added to the antibody or antigen-binding fragment thereof to improve its stability (particularly where the antibody is an antibody fragment such as an Fv fragment).

Nucleic acid molecules encoding amino acid sequence variants of an anti-LAYN antibody or antigen-binding fragment thereof can be prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-LAYN antibody or antigen-binding fragment thereof. For example, nucleic acid molecules according to the invention also encompass nucleic acid molecules which hybridize under stringent conditions to nucleic acid molecules as disclosed herein, whereby the term “stringent conditions” within the scope of the invention can include, e.g., hybridization in a buffer comprising 50% formamide, 5×SSC, and 1% SDS at 42 °C, or hybridization in a buffer comprising 5×SSC and 1% SDS at 65 °C, both with a wash of 0.2×SSC and 0.1% SDS at 65 °C. Exemplary stringent hybridization conditions can also include a hybridization in a buffer of 40% formamide, 1 M NaCl, and 1% SDS at 37 °C, and a wash in 1×SSC at 45 °C.

As used herein, the terms “identical” or “percent identity,” in the context of two or more nucleic acids or polypeptide (antibody) sequences, refer to two or more sequences or subsequences that are the same or have a specified percentage of nucleotides or amino acid residues that are the same, when compared and aligned for maximum correspondence. Accordingly, “percent identity” of one sequence is typically determined by comparison to a reference sequence. In the context of the present specification, reference sequences for variant anti-LAYN antibodies, and antigen-binding fragments thereof, are preferably the sequences of exemplary antibodies P and A1-A104, preferably P and A1-A97, more preferably A1-A88, A90 and A91 (e.g., as depicted in Tables 2-8). To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). For example, if 92 out of 100 positions are identical in two sequences, these sequences share 92% identity. In some aspects, the two sequences that are compared are the same length after gaps are introduced within the sequences, as appropriate (e.g., excluding additional sequence extending beyond the sequences being compared). For example, when variable region sequences are compared, the leader and/or constant domain sequences are not considered. For sequence comparisons between two sequences, a “corresponding” CDR refers to a CDR in the same location in both sequences (e.g., HCDR1 of each sequence).

The determination of percent identity or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to a protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, 1988, CABIOS 4(1):11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torelli and Robotti, 1994, CABIOS 10(1):3-5; and FASTA described in Pearson and Lipman, 1988, Proc. Natl. Acad. Sci. USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

In general, the anti-LAYN antibodies of the present disclosure, or antigen-binding fragments thereof, or one or more domains of antibodies, are usually recombinantly expressed. Accordingly, the antibody, or antigen-binding fragment thereof, may be referred to as “recombinant”. As used herein, the term “recombinant” refers to heterologous expression, i.e. expression of a polypeptide or protein in a host cell or organism that does not naturally express that polypeptide or protein (e.g., due to a lack of a respective nucleic acid sequence encoding the polypeptide or protein). A non-limiting example of heterologous (or recombinant) expression is the expression of a protein from one species in (a cell of) another species. In general, recombinant expression may employ one or more control sequences, i.e., polynucleotide sequences necessary for expression of an operably linked coding sequence in a particular host cell or organism. The control sequences suitable for use in prokaryotic cells include, for example, promoter, operator, and ribosome binding site sequences. Eukaryotic control sequences include, but are not limited to, promoters, polyadenylation signals, and enhancers. These control sequences can be utilized for expression and production of antibodies or antigen-binding fragments thereof in prokaryotic and eukaryotic host cells.

A nucleic acid sequence is “operably linked” when it is placed into a functional relationship with another nucleic acid sequence. For example, a nucleic acid presequence or secretory leader is operably linked to a nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers are optionally contiguous. Linking can be accomplished by ligation at convenient restriction sites. If such sites do not exist, synthetic oligonucleotide adaptors or linkers can be used.

As used herein, the expressions “cell”, “cell line”, and “cell culture” are used interchangeably and all such designations include the progeny thereof. Thus, “transformants” and “transformed cells” include the primary subject cell and cultures derived therefrom without regard for the number of transfers, which may for example have been transfected with one or more expression vectors encoding one or more amino acids sequences of an antibody or antigen-binding fragment thereof of the present invention.

The term “mammal”, in particular for purposes of treatment, refers to any animal classified as a mammal, including humans, domesticated and farm animals, and zoo, sports, or companion animals, such as dogs, horses, cats, cows, and the like. Preferably, the mammal is a human.

A “disorder”, as used herein, is any condition that would benefit from treatment with an anti-LAYN antibody or antigen-binding fragment thereof described herein. This includes chronic and acute disorders or diseases including those pathological conditions that predispose the mammal to the disorder in question. Non-limiting examples or disorders to be treated herein include cancers.

The term “subcutaneous administration” refers to introduction of a drug, for example an anti-LAYN antibody or antigen-binding fragment thereof, under the skin of a subject such as an animal or human patient. Subcutaneous administration may be performed within a pocket between the skin and underlying tissue. Subcutaneous administration may be performed by relatively slow, sustained delivery from a drug receptacle. Pinching or drawing the skin up and away from underlying tissue may create the pocket.

The term “(therapeutically) effective amount” of an agent, e.g., a pharmaceutical composition, refers to an amount effective, at dosages and for periods of time necessary, to achieve the desired therapeutic or prophylactic result. Typically, a “(therapeutically) effective amount” refers to an amount of an agent, e.g., a pharmaceutical composition, that relieves or ameliorates one or more of the symptoms of the disorder being treated. In doing so, it is that amount that has a beneficial patient outcome. Efficacy can be measured in conventional ways, depending on the condition to be treated.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an antibody or antigen-binding fragment thereof prior to or following the onset of a symptom of a disease or disorder thereby preventing or removing one or more signs of the disease or disorder. As an example, the term includes the administration of an antibody or antigen-binding fragment thereof after clinical manifestation of the disease to combat the symptoms of the disease. Further, administration of an antibody or antigen-binding fragment thereof after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the antibody or antigen-binding fragment thereof composition or antigen-binding fragment thereof, the result should be considered an effective treatment of the underlying disorder regardless of whether all the symptoms of the disorder are alleviated or not.

To “prevent” refers to prophylactic or preventative measures that prevent and/or slow/delay the development of a targeted pathologic condition or disorder. It also includes the reduction of occurrence of a targeted pathologic condition or disorder. Thus, those in need of prevention include those prone to have or susceptible to the disorder. In some embodiments, a disease or disorder is successfully prevented, as described herein, if the patient develops, transiently or permanently, e.g., fewer or less severe symptoms associated with the disease or disorder, or a later onset of symptoms associated with the disease or disorder, than a patient who has not been subject to the methods of the invention.

In the following, the elements of the present disclosure will be described. These elements may be listed with specific embodiments, however, it should be understood that they may be combined in any manner and in any number to create additional embodiments. The variously described Examples and embodiments should not be construed to limit the present disclosure to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present specification unless the context indicates otherwise.

### Antibodies and Antigen-Binding Fragments Thereof

The present inventors have surprisingly found anti-LAYN antibodies, and antigen-binding fragments thereof, which (i) bind to LAYN-expressing cells and (ii) show a high ADCC potential on LAYN-expressing cells, as demonstrated in Examples 22 and 23 of the present specification. Without being bound to any theory, the present inventors assume that the antibody's potential to induce ADCC on LAYN-expressing cells is an important prerequisite to mediate depletion of such LAYN-expressing cells, in particular intra-tumoral Tregs. In addition, anti-LAYN antibodies, and antigen-binding fragments thereof of the present disclosure (i) specifically bind (with a K_{D} value of less than 50 nM) to the extracellular domain (ECD) of LAYN isoform 2, e.g., having the amino acid sequence of SEQ ID NO: 191 (or SEQ ID NO: 190) and (ii) show high stability, in particular having a melting temperature (Tm) of the Fab fragment of said antibodies of at least 80 °C. In particular, anti-LAYN antibodies, and antigen-binding fragments thereof of the present disclosure:
(i) specifically bind (with a K_{D} value of less than 50 nM) to the extracellular domain (ECD) of LAYN isoform 2, e.g., having the amino acid sequence of SEQ ID NO: 190 or 191;
(ii) specifically bind (with a K_{D} value of less than 50 nM) to the ECD of LAYN isoform 1, e.g., having the amino acid sequence of SEQ ID NO: 196 or 197; and
(iii) show high stability, in particular having a melting temperature (Tm) of the Fab fragment of said antibodies of at least 80 °C.

As shown in Example 18 of the present specification, in contrast to the antibodies of the present disclosure, none of the tested prior art antibodies demonstrated both features, namely, a high binding affinity to LAYN isoform 2 (e.g., with a K_{D} value of less than 50 nM) and a high stability (e.g., with a Tm Fab of at least 80 °C). Moreover, the antibodies of the present disclosure specifically bind to LAYN, in particular (the ECD of) human LAYN isoform 2, not only at (neutral) pH 7.4, but also at the slightly acidic pH of 6.0, which is commonly observed in the tumor microenvironment due to the accumulation of acidic metabolic waste products (Boedtkjer E, Pedersen S F. The Acidic Tumor Microenvironment as a Driver of Cancer. Annu Rev Physiol. 2020 Feb. 10; 82:103-126. doi: 10.1146/annurev-physiol-021119-034627. Epub 2019 Nov. 15). Furthermore, the antibodies of the present disclosure demonstrated binding to human regulatory T cells (Tregs) and depletion of human Tregs as well as efficacy in an in vivo mouse tumor model.

In some aspects, the present disclosure provides an antibody or an antigen-binding fragment thereof, which specifically binds to human LAYN, in particular to isoform 2 of human LAYN, such as full length hLAYN isoform 2 (e.g., having the amino acid sequence of SEQ ID NO: 194) or the extracellular domain (ECD) of LAYN (e.g., having the amino acid sequence of SEQ ID NO: 190 or SEQ ID NO: 191). In some aspects, the antibody or an antigen-binding fragment thereof, according to the present disclosure specifically binds to a truncated ECD human LAYN isoform 2, e.g. having an amino acid sequence of amino acid residues 1-156 of SEQ ID NO: 192 (or having the amino acid sequence of SEQ ID NO: 192, which includes, in addition to amino acid residues 1-156, a linker and a His-tag).

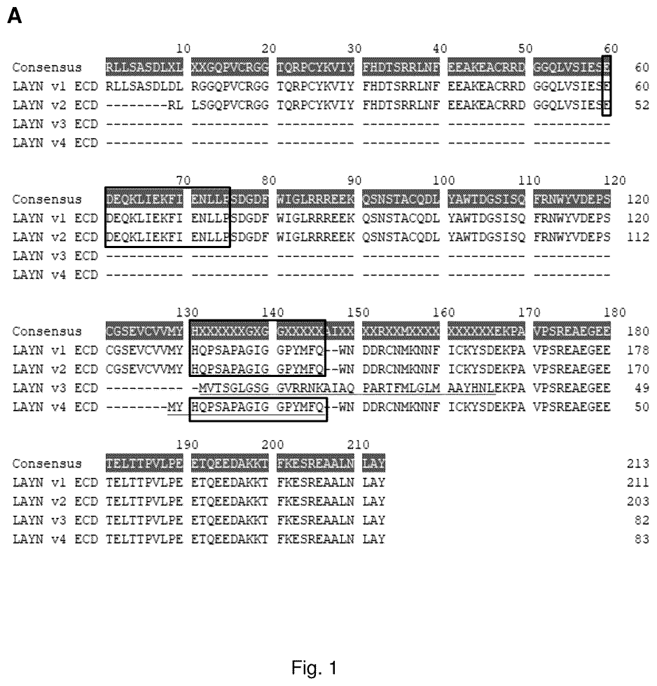

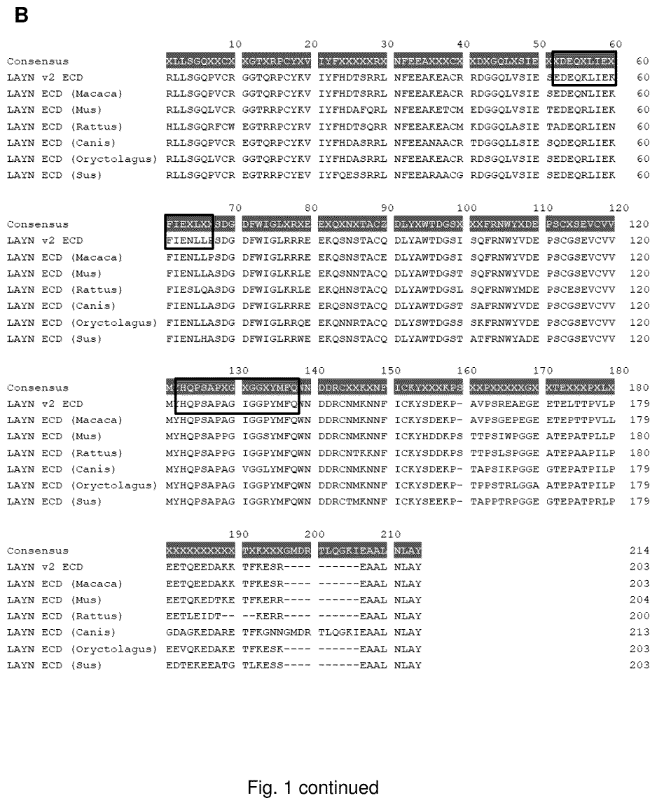

In some aspects, the antibodies or antigen-binding fragments thereof, as disclosed herein specifically bind to human LAYN, in particular to isoform 1 and/or 2 of human LAYN, such as full length hLAYN isoform 2 (e.g., having the amino acid sequence of SEQ ID NO: 194). More specifically, the antibodies, or antigen-binding fragments thereof, as disclosed herein may (specifically) bind to the extracellular domain (ECD) of human LAYN isoform 2 (e.g., having the amino acid sequence of SEQ ID NO: 191 or 190) and/or to the extracellular domain (ECD) of human LAYN isoform 1 (e.g., having the amino acid sequence of SEQ ID NO: 197 or 196). In some aspects, the antibodies, or antigen-binding fragments thereof, as disclosed herein do not (specifically) bind to (the ECD of) human LAYN isoform 3 (e.g., having the amino acid sequence of SEQ ID NO: 198) and/or to (the ECD of) human LAYN isoform 4 (e.g., having the amino acid sequence of SEQ ID NO: 199). An alignment of the extracellular domains of human LAYN isoforms 1, 2, 3 and 4 is shown in FIG. 1A.

In some aspects, the antibodies of the present disclosure, or the antigen-binding fragments thereof, bind to (the ECD of) one or more of
human LAYN isoform 1 (e.g., (comprising) an ECD having the amino acid sequence of SEQ ID NO: 197 or 196),
human LAYN isoform 2 (e.g., having the amino acid sequence of SEQ ID NO: 193 or 194; or (comprising) an ECD having the amino acid sequence of SEQ ID NO: 191 or 190),
an ortholog of human LAYN isoform 2 in cynomolgus (e.g., NCBI Reference Sequence (RefSeq) #XP_005579631.3, Macaca fascicularis, or (comprising) an ECD having the amino acid sequence of SEQ ID NO: 200),
an ortholog of human LAYN isoform 2 in mouse (e.g., RefSeq #XP_006510392.1, Mus musculus, or (comprising) an ECD having the amino acid sequence of SEQ ID NO: 203),
an ortholog of human LAYN isoform 2 in rabbit (e.g., RefSeq #XP_008259820.2, Oryctolagus cuniculus, or (comprising) an ECD having the amino acid sequence of SEQ ID NO: 204),
an ortholog of human LAYN isoform 2 in rat (e.g., RefSeq #XP_006243111.1, Rattus norvegicus, or (comprising) an ECD having the amino acid sequence of SEQ ID NO: 202), and
an ortholog of human LAYN isoform 2 in dog (e.g., RefSeq #XP_038520860.1, Canis lupus familiaris, or (comprising) an ECD having the amino acid sequence of SEQ ID NO: 201).

In some aspects, the antibodies of the present disclosure, or the antigen-binding fragments thereof, bind to (the ECD of) all of:
human LAYN isoform 1 (e.g., (comprising) an ECD having the amino acid sequence of SEQ ID NO: 197 or 196),
human LAYN isoform 2 (e.g., having the amino acid sequence of SEQ ID NO: 193 or 194; or (comprising) an ECD having the amino acid sequence of SEQ ID NO: 191 or 190),
an ortholog of human LAYN isoform 2 in cynomolgus (e.g., NCBI Reference Sequence (RefSeq) #XP_005579631.3, Macaca fascicularis, or (comprising) an ECD having the amino acid sequence of SEQ ID NO: 200),
an ortholog of human LAYN isoform 2 in mouse (e.g., RefSeq #XP_006510392.1, Mus musculus, or (comprising) an ECD having the amino acid sequence of SEQ ID NO: 203),
an ortholog of human LAYN isoform 2 in rabbit (e.g., RefSeq #XP_008259820.2, Oryctolagus cuniculus, or (comprising) an ECD having the amino acid sequence of SEQ ID NO: 204),
an ortholog of human LAYN isoform 2 in rat (e.g., RefSeq #XP_006243111.1, Rattus norvegicus, or (comprising) an ECD having the amino acid sequence of SEQ ID NO: 202), and
an ortholog of human LAYN isoform 2 in dog (e.g., RefSeq #XP_038520860.1, Canis lupus familiaris, or (comprising) an ECD having the amino acid sequence of SEQ ID NO: 201).

In some aspects, the antibodies of the present disclosure, or the antigen-binding fragments thereof, do not (specifically) bind to (the ECD of) one or more of:
human LAYN isoform 3 (e.g., (comprising) an ECD having the amino acid sequence of SEQ ID NO: 198),
human LAYN isoform 4 (e.g., (comprising) an ECD having the amino acid sequence of SEQ ID NO: 199), and
an ortholog of human LAYN isoform 2 in pig (e.g., RefSeq #NP_001231823.1, Sus scrofa, or (comprising) an ECD having the amino acid sequence of SEQ ID NO: 205).

In some aspects, the antibodies of the present disclosure, or the antigen-binding fragments thereof, do not (specifically) bind to (the ECD of) all of:
human LAYN isoform 3 (e.g., (comprising) an ECD having the amino acid sequence of SEQ ID NO: 198),
human LAYN isoform 4 (e.g., (comprising) an ECD having the amino acid sequence of SEQ ID NO: 199), and
an ortholog of human LAYN isoform 2 in pig (e.g., RefSeq #NP_001231823.1, Sus scrofa, or (comprising) an ECD having the amino acid sequence of SEQ ID NO: 205).

An alignment of the extracellular domains of human LAYN isoform 2 and its respective orthologs in cynomolgus, dog, rat, mouse, rabbit and pig are shown in FIG. 1B.

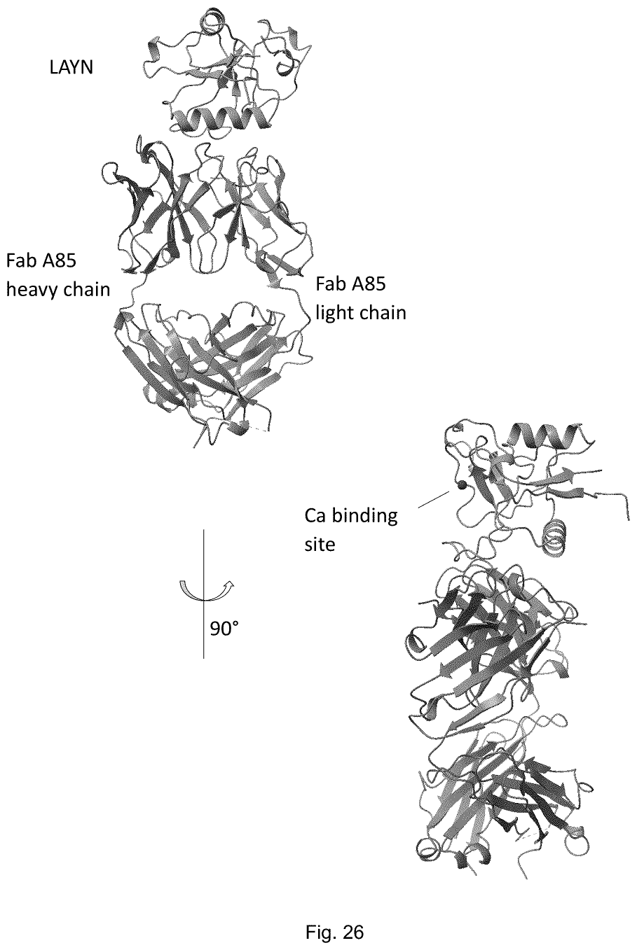

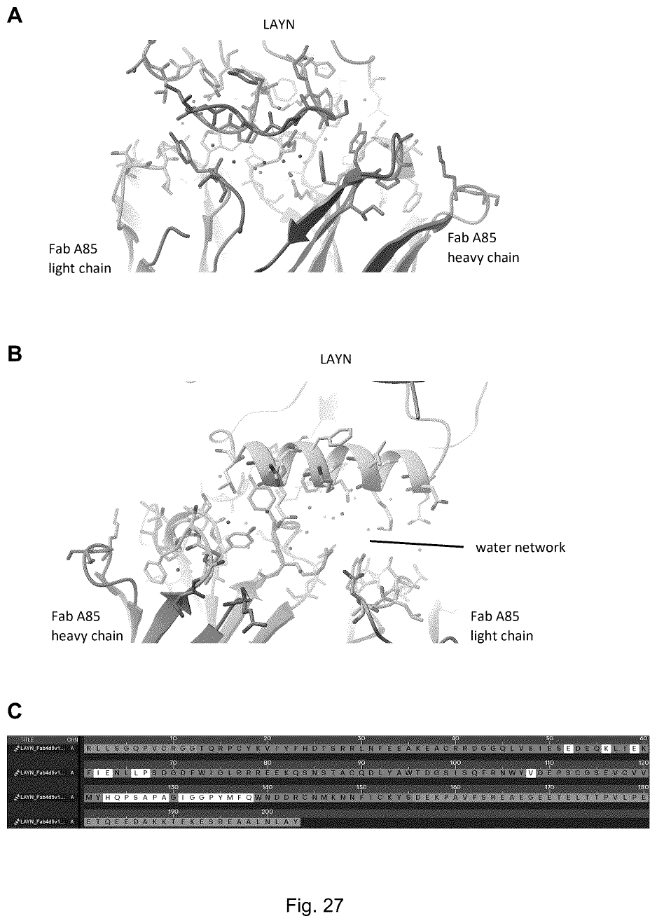

In some aspects, the present disclosure provides an anti-LAYN antibody, or an antigen-binding fragment thereof, which binds to an epitope of the extracellular domain of human LAYN isoform 2 (e.g., having the amino acid sequence of SEQ ID NO: 191), wherein the epitope comprises (i) amino acid residues comprised in the loop of human LAYN isoform 2 containing residues 123-138 of SEQ ID NO: 191, and (ii) amino acid residues comprised in the helix of human LAYN isoform 2 containing residues 52-67 of SEQ ID NO: 191. In other words, the present disclosure provides an anti-LAYN antibody, or an antigen-binding fragment thereof, which binds to an epitope of the extracellular domain of LAYN isoform 2 (e.g., having the amino acid sequence of SEQ ID NO: 191), wherein the epitope comprises one or more amino acid residues selected from residues 123-138 of SEQ ID NO: 191, and one or more amino acid residues selected from residues 52-67 of SEQ ID NO: 191. Example 16 herein below describes how the epitope has been determined (illustrated by FIGS. 26 and 27). As shown in Example 16, not each and every amino acid residue of in the loop of human LAYN isoform 2 containing residues 123-138 of SEQ ID NO: 191, and of the helix of human LAYN isoform 2 containing residues 52-67 of SEQ ID NO: 191 interacts directly with the paratope.

As described in Example 16, it has been determined that amino acid residues E63, H123, P125 and A127 of LAYN (SEQ ID NO: 191) form direct hydrogen bonds with the antibody. Accordingly, in some aspects, the epitope may comprise (i) amino acid residues H123, P125 and A127 of SEQ ID NO: 191, and (ii) amino acid residue E63 of SEQ ID NO: 191. In some aspects, the antibody of the present disclosure, or the antigen-binding fragment thereof, (has the ability to) form hydrogen bonds with amino acid residues E63, H123, P125 and A127 of LAYN (SEQ ID NO: 191). Preferably, the epitope comprises amino acid residues E52, K56, E59, 162, E63, L66, P67, H123, Q124, P125, S126, A127, P128, A129, I131, G132, G133, P134, Y135, M136, F137 and Q138 of SEQ ID NO: 191. Additionally, the epitope may comprise V108 of SEQ ID NO: 191. More preferably, the epitope comprises amino acid residues E52, K56, E59, 162, E63, L66, P67, V108, H123, Q124, P125, S126, A127, P128, A129, I131, G132, G133, P134, Y135, M136, F137 and Q138 of SEQ ID NO: 191. In some aspects, the antibody of the present disclosure, or the antigen-binding fragment thereof, (has the ability to) bind to LAYN, in particular to SEQ ID NO: 191, in such a manner that amino acid residues of the antibody, or the antigen-binding fragment, have a distance of no more than 4.0 Å to amino acid residues E52, K56, E59, 162, E63, L66, P67, V108, H123, Q124, P125, S126, A127, P128, A129, I131, G132, G133, P134, Y135, M136, F137 and Q138 of LAYN (SEQ ID NO: 191).

In some aspects, the present disclosure provides an anti-LAYN antibody, or an antigen-binding fragment thereof, which specifically binds to human LAYN, in particular human LAYN isoform 2, at pH 6. Moreover, the antibodies or the antigen-binding fragments thereof may specifically bind to LAYN, in particular (the ECD of) human LAYN isoform 2 (e.g., comprising or consisting of the amino acid sequence of SEQ ID NO: 190 or 191), at pH 7.4 and at pH 6. A pH of 6.0 is commonly observed in the tumor microenvironment. Therefore, an antibody binding to LAYN at pH 6 is particularly useful when binding to LAYN in the tumor microenvironment is required, e.g., in the treatment of a cancer.

In some embodiments, the antibody or the antigen-binding fragment specifically binds to human LAYN after storage for at least four weeks at a temperature of 40 °C. In some embodiments, the antibody or the antigen-binding fragment specifically binds to human LAYN after storage for at least four weeks at a concentration of the antibody or the antigen-binding fragment of 150 g/l. In some embodiments, the antibody or the antigen-binding fragment specifically binds to human LAYN after storage for at least four weeks at a temperature of 40 °C and at a concentration of the antibody or the antigen-binding fragment of 150 g/l. In some embodiments, the antibody or the antigen-binding fragment specifically binds to human LAYN after storage for at least four weeks at a temperature of 40 °C, at pH 6 and at a concentration of the antibody or the antigen-binding fragment of 150 g/l.

In some aspects, the antibody binds to LAYN, such as human LAYN, in particular (the ECD of) human LAYN isoform 2 (e.g., having an amino acid sequence of SEQ ID NO: 190 or 191), at pH 6 with an affinity as determined by surface plasmon resonance analysis (SPR; □almqvist □., “Surface plasmon resonance for detection and measurement of antibody-antigen affinity and kinetics.”, Curr Opin Immunol. 1993 April; 5(2):282-6.). In some embodiments, the antibody binds to LAYN, such as human LAYN, in particular (the ECD of) human LAYN isoform 2 (e.g., having an amino acid sequence of SEQ ID NO: 190 or 191), at pH 6 with high affinity as determined by SPR. A specific exemplified method for performing SPR is described in Example 3. Preferably, the K_{D} value obtained by SPR for binding of the antibody to the antigen at pH 6 ranges from 1 pM to 100 μM, more preferably 1 pM to 1 μM. In some embodiments, the binding affinity at pH 6 is characterized by a K_{D} of about 1×10⁻⁷ M (100 nM) or less. In some embodiments, the binding affinity at pH 6 is characterized by a K_{D} of about 5×10⁻⁸ M (50 nM) or less. In some embodiments, high affinity as described herein includes wherein the binding affinity obtained by SPR for binding of the antibody to the antigen at pH 6 is characterized by a K_{D} of about 5×10⁻⁸ M (50 nM) or less. In embodiments, high affinity as described herein includes wherein the K_{D} value (binding affinity) obtained by SPR for binding of the antibody to the antigen at pH 6 is between about 0.001 nM and 50 nM, between about 0.01 nM and 50 nM, between about 0.1 nM and 50 nM, between about 1 nM and 50 nM, or less than about 50 nM.

In some aspects, the antibody or the antigen binding fragment has a (high) stability as indicated by a melting temperature Tm or an inflection point (IP) of a thermal transition curve of the Fab fragment of the antibody, or the antigen-binding fragment, of at least 80 °C. The present invention also provides an anti-LAYN antibody, or an antigen-binding fragment thereof, specifically binding to human LAYN, in particular human LAYN isoform 2 and/or human LAYN isoform 1 (but not to human LAYN isoform 3 or 4), wherein the Fab fragment of the antibody, or the antigen-binding fragment, has a melting temperature Tm or an inflection point (IP) of a thermal transition curve of at least 80 °C. In some embodiments, the present disclosure provides an anti-LAYN antibody, or an antigen-binding fragment thereof, specifically binding to human LAYN, in particular human LAYN isoform 2 and/or human LAYN isoform 1 (but not to human LAYN isoform 3 or 4), wherein the Fab fragment of the antibody, or the antigen-binding fragment, has high stability, for example, a melting temperature Tm or an inflection point (IP) of a thermal transition curve of at least 80 °C. In embodiments, high stability includes wherein the melting temperature or an inflection point (IP) of a thermal transition curve is between about 80 °C and 99 °C, between about 80 °C and 95 °C, between about 80 °C and 90 °C, or between about 80 °C and 85 °C.

The melting temperature Tm or the inflection point (IP) of a thermal transition curve of a Fab fragment can be determined by different thermal unfolding methods known in the art, including differential scanning calorimetry (DSC) and differential scanning fluorimetry (DSF). In general, these methods rely on characterization of thermal unfolding of a protein when a gradient of increasing temperature (heating) is applied. The applied temperature gradient typically starts at a temperature well below the onset of the protein unfolding (e.g., 25 °C) and rises up to temperatures at which thermal unfolding has occurred (e.g., 90 or 95 °C). DSC follows protein unfolding by measuring the heat capacity of protein states and the excess heat associated with transitions that are induced by a change in temperature. DSF relies on changes in the fluorescence either of an environmentally sensitive dye, such as Sypro® Orange, partitioning itself into the unfolded protein. A variation thereof, known as “nanoDSF”, does not require the addition of a dye, but relies on fluorescence changes of tryptophan or tyrosine residues in the protein itself.

In DSC and DSF, the changes (in heat capacity/excess heat in DSC or in fluorescence in DSF) are usually recorded depending on the temperature. Thereby, so-called “thermal transition” curves can be obtained. These curves can be used to determine characteristic parameters of the thermal unfolding of the protein, such as the temperature at which unfolding begins (Tₒₙₛₑₜ) and/or the midpoint of transition, i.e. the temperature at which 50% of the protein is unfolded (also referred to as “melting temperature” or Tm). Usually, the inflection point (IP) of the thermal unfolding curve corresponds essentially to the “melting temperature” (Tm), which is the midpoint of transition, i.e. the temperature at which 50% of the protein is unfolded.

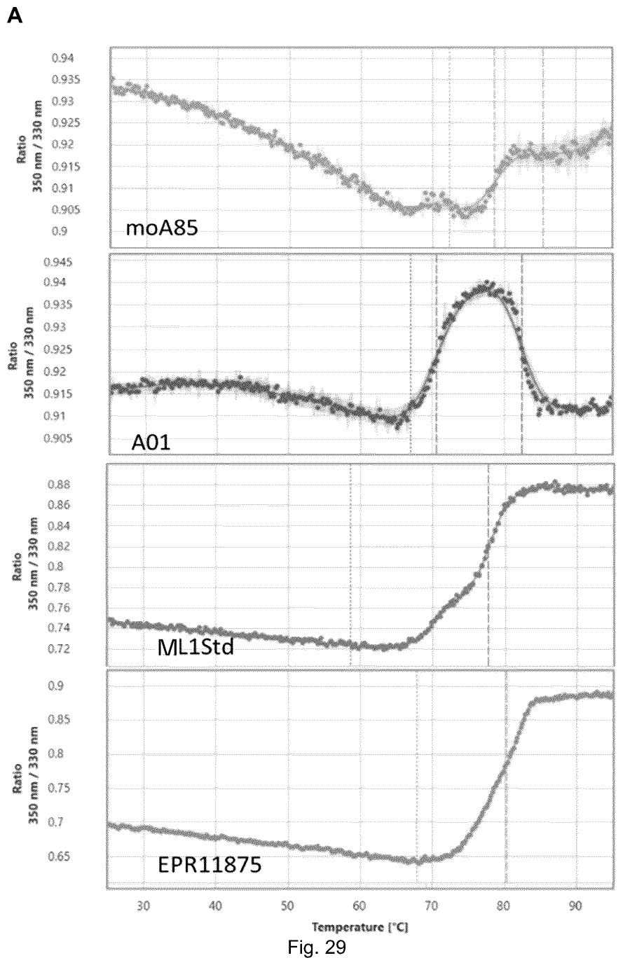

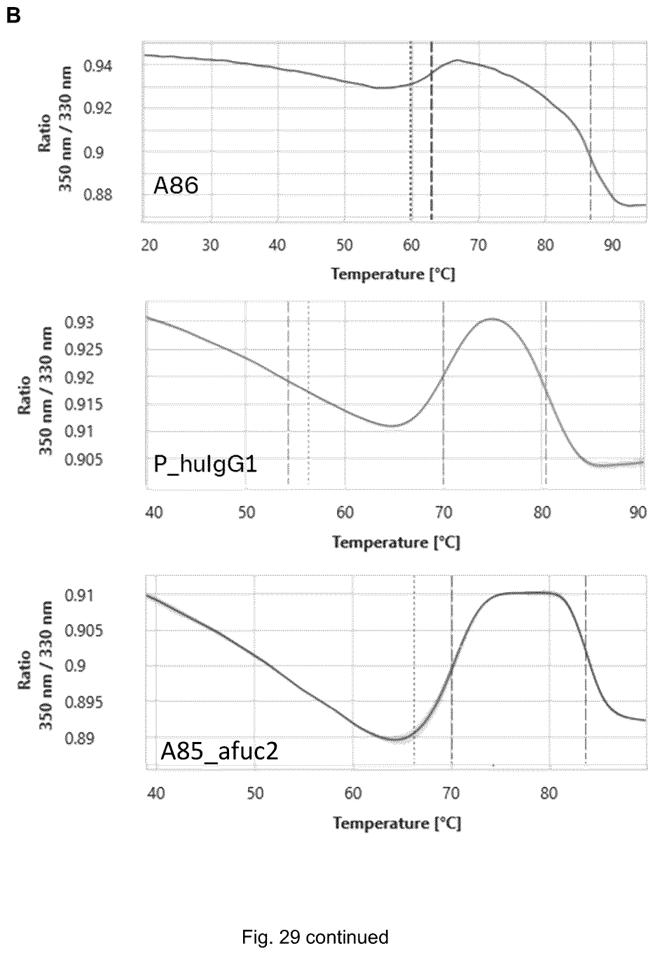

Preferably, DSF or nanoDSF is used to determine the melting temperature Tm or the inflection point (IP) of a thermal transition curve of a Fab fragment of the anti-LAYN antibody. A specific DSF method is described in Example 4 and a specific nanoDSF method is described in Example 18. NanoDSF is even more preferred to determine the melting temperature Tm or the inflection point (IP) of a thermal transition curve of a Fab fragment of the anti-LAYN antibody. For example, in nanoDSF, thermal unfolding can be determined based on tracking of the intrinsic antibody fluorescence (from Trp and Tyr residues) at 350 nm and 330 nm, respectively. The ratio of the fluorescence at 350 to 330 nm (F350 nm/F330 nm) may then be used to obtain a thermal transition curve (see exemplary curves in FIG. 29 as illustrative examples). The inflection point of those curves can be readily determined by the person skilled in the art. Usually, the inflection point (IP) of the curve corresponds essentially to the “melting temperature” (Tm), which is the midpoint of transition, i.e. the temperature at which 50% of the protein is unfolded.

In some aspects, the present disclosure provides an anti-LAYN antibody, or an antigen-binding fragment thereof, specifically binding to human LAYN, in particular human LAYN isoform 2 and/or human LAYN isoform 1 (but not to human LAYN isoform 3 or 4), wherein the Fab fragment of the antibody, or the antigen-binding fragment, has a melting temperature Tm or an inflection point (IP) of a thermal transition curve of at least 80 °C. In some embodiments, the antibody, or the antigen-binding fragment thereof,
(i) binds to LAYN, such as human LAYN, in particular (the ECD of) human LAYN isoform 2 (e.g., having an amino acid sequence of SEQ ID NO: 190 or 191) with an affinity characterized by a K_{D} of about 5×10⁻⁸ M (50 nM) or less (as determined by SPR); and
(ii) the Fab fragment of the antibody, or the antigen-binding fragment, has a melting temperature Tm or an inflection point (IP) of a thermal transition curve of at least 80 °C (as determined by nanoDSF).

In some aspects, the present disclosure provides an anti-LAYN antibody, or an antigen-binding fragment thereof, that (i) specifically binds to cells expressing human LAYN isoform 2 and (ii) shows ADCC on cells expressing human LAYN isoform 2. This is demonstrated for exemplified antibodies in Examples 22 and 23. Accordingly, an exemplified method for testing the binding of an antibody to LAYN expressing cells is described in Example 22 and an exemplified method for testing ADCC on cells expressing human LAYN is described in Example 23. Preferably, the anti-LAYN antibody, or antigen-binding fragment thereof, is shows an EC50 of less than 0.15 nM in an ADCC reporter assay (e.g., as described in Example 23), in particular wherein Jurkat cells expressing full length LAYN isoform 2 (SEQ ID NO: 193) are co-cultured in a 1:1 ratio with CD16AV158-expressing Jurkat NFAT luciferase reporter cells (Promega, Madison, WI) at 37 °C for 4 h in the presence of the antibody to be tested; BIO-GLO™ (Promega, Madison, WI) is added to the cells and then the luminescence is read for measurement of luciferase activity and the half-maximal effective concentration (EC50) is calculated. More preferably, the EC50 of the anti-LAYN antibody in an ADCC reporter assay (as described herein) is less than 0.1 nM, such as less than 0.09 nM, less than 0.08 nM, less than 0.07 nM, less than 0.06 nM, less than 0.05 nM, less than 0.04 nM or less than 0.03 nM, whereby the lower the EC50 the more preferred is the antibody. Particularly preferably, the EC50 in an ADCC reporter assay (as described herein) is less than 0.025 nM. Without being bound to any theory, the present inventors assume that the antibody's potential to induce ADCC on LAYN-expressing cells is an important prerequisite to mediate depletion of such LAYN-expressing cells, in particular intra-tumoral Tregs.

In some embodiments, the anti-LAYN antibody, or the antigen-binding fragment binds to human regulatory T cells (Tregs) and/or mediates depletion of human regulatory T cells. Without being bound to any theory, it is assumed that the isoform 2 of LAYN is primarily expressed and/or upregulated in intratumoral regulatory T cells. By binding to said intratumoral Tregs, the anti-LAYN antibody, or the antigen-binding fragment, can reduce or deplete the Tregs. Without being bound to any theory, it is assumed that thereby an immune response against the tumor can be elicited or increased. The Examples appended hereto demonstrate that exemplary antibodies of the present disclosure bind to human regulatory T cells (Tregs) and mediate depletion of human Tregs. The Examples also show the efficacy of exemplary antibodies of the present disclosure in a mouse tumor model.

Accordingly, the present disclosure also provides a (in-vitro, ex-vivo or in-vivo) method of reducing or depleting intratumoral regulatory T cells, the method comprising the use of the anti-LAYN antibody, or an antigen-binding fragment thereof, as described herein.

### Structure of Antibodies and Antigen-Binding Fragments

The generalized structure of antibodies or immunoglobulin is well known to those of skill in the art, these molecules are heterotetrametric glycoproteins, typically of about 150,000 daltons, composed of two identical light (L) chains and two identical heavy (H) chains. Each light chain is covalently linked to a heavy chain by one disulfide bond to form a heterodimer, and the heterotrimeric molecule is formed through a covalent disulfide linkage between the two identical heavy chains of the heterodimers. Although the light and heavy chains are linked together by one disulfide bond, the number of disulfide linkages between the two heavy chains varies by immunoglobulin isotype. Each heavy and light chain also has regularly spaced intra-chain disulfide bridges. Each heavy chain has at the amino-terminus a variable domain (VH; heavy chain variable region), followed by three or four constant domains (CH1, CH2, CH3, and, optionally, CH4) and includes a hinge region between CH1 and CH2. Each light chain has two domains, an amino-terminal variable domain (VL; light chain variable region) and a carboxy-terminal constant domain (CL). The VL domain associates non-covalently with the VH domain, whereas the CL domain is commonly covalently linked to the CH1 domain via a disulfide bond. Particular amino acid residues are believed to form an interface between the light and heavy chain variable domains (Chothia et al., 1985, J. Mol. Biol. 186:651-663, Vargas-Madrazo E, Paz-García E. J Dol Recognit. 2003; 16(3):113-120). The variable domains are also referred herein as variable regions, and the constant domains as constant regions.

Certain domains within the variable domains differ extensively between different antibodies i.e., are “hypervariable.” These hypervariable domains contain residues that are directly involved in the binding and specificity of each particular antibody for its specific antigenic determinant. Hypervariability, both in the light chain and the heavy chain variable domains, is concentrated in three segments known as complementarity determining regions (CDRs) or hypervariable loops (HVLs). The CDR1, CDR2, CDR3 of the heavy and light chains therefore define the unique and functional properties specific for a given antibody.

The three CDRs within each of the heavy and light chains are separated by framework regions (FR), which contain sequences that tend to be less variable. From the amino terminus to the carboxy terminus of the heavy and light chain variable domains, the FRs and CDRs are arranged in the order: FR1, CDR1, FR2, CDR2, FR3, CDR3, and FR4. The largely β-sheet configuration of the FRs brings the CDRs within each of the chains into close proximity to each other as well as to the CDRs from the other chain. The resulting conformation contributes to the antigen binding site (see Kabat et al., Sequences of Proteins of Immunological Interest, Vol. 1, 5th ed., 1991, Bethesda, MD: U.S. Dept. of Health and Human Services, Public Health Service, National Institutes of Health, NIH Publication No. 91-3242), although not all CDR residues are necessarily directly involved in antigen binding. Those skilled in the art can routinely determine which residues comprise a particular CDR given the variable region amino acid sequence of the antibody. The CDR1, CDR2, CDR3 of the heavy and light chains therefore define the unique and functional properties specific for a given antibody.

Various approaches for defining CDRs are known in the art. Unless stated otherwise, the CDR sequences described herein are defined by the method of sequence comparison described originally in Kabat et al., Sequences of Proteins of Immunological Interest, Vol. 1, 5th ed., 1991, Bethesda, MD: U.S. Dept. of Health and Human Services, Public Health Service, National Institutes of Health, NIH Publication No. 91-3242, whereas HVLs are structurally defined according to the three-dimensional structure of the variable domain, as described by Chothia and Lesk, 1987, J. Mol. Biol. 196:901-917.

As defined by Kabat, LCDR1 is positioned at about residues 24-34, LCDR2, at about residues 50-56, and LCDR3, at about residues 89-97 in the light chain variable domain; HCDR1 is positioned at about residues 31-35, HCDR2 at about residues 50-65, and HCDR3 at about residues 95-102 in the heavy chain variable domain. Accordingly, according to the Kabat numbering, the resulting positions for FRs and CDRs are usually as follows for the light chain:
FR1 comprises the amino acid residues at positions 1-23,
CDR1 comprises the amino acid residues at positions 24-34,
FR2 comprises the amino acids at positions 35-49,
CDR2 comprises the amino acid residues at positions 50-65,
FR3 comprises the amino acid residues at positions 66-88,
CDR3 comprises the amino acid residues at positions 89-97, and
FR4 comprises the amino acid residues at positions 98-113;
   and as follows for the heavy chain:
FR1 comprises the amino acid residues at positions 1-30,
CDR1 comprises the amino acid residues at positions 31-35,
FR2 comprises the amino acids at positions 36-49,
CDR2 comprises the amino acid residues at positions 50-65,
FR3 comprises the amino acid residues at positions 66-94,
CDR3 comprises the amino acid residues at positions 95-102, and
FR4 comprises the amino acid residues at positions 103-113.

However, it should be noted that—as is well known in the art for VH domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence.

Described and disclosed herein are anti-LAYN antibodies, in particular human anti-LAYN antibodies, as well as compositions and articles of manufacture comprising anti-LAYN antibodies of the present invention. Also described are antigen-binding fragments of an anti-LAYN antibodies. An anti-LAYN antibody and an antigen-binding fragment thereof each include at least a portion that specifically binds to LAYN, or a fragment thereof, e.g., the ECD of LAYN, preferably human LAYN, more preferably the ECD of human LAYN isoform 2.

The generation of exemplary anti-LAYN antibodies and their characterization is described in the Examples. CDR, VH and VL as well as full-length heavy chain (HC) and light chain sequences of exemplary antibodies of the present disclosure are described in Table 2 below.

TABLE 2
SEQ ID NOs of amino acid sequences of the six CDRs, heavy chain variable
region (VH), light chain variable region (VL), heavy chain (HC) and light
chain (LC) of each of exemplary antibodies (mAb) A1-A104 and P. Antibodies
A01, A02, A03, A04, A05, A06, A07, A08 and A09 are also referred to herein
as antibodies A1, A2, A3, A4, A5, A6, A7, A8 and A9, respectively.

| | Heavy chain SEQ ID NOs | | | | | Light chain SEQ ID NOs | | | | |
|---|---|---|---|---|---|---|---|---|---|---|
| mAb | CDR1 | CDR2 | CDR3 | VH | HC | CDR1 | CDR2 | CDR3 | VL | LC |
| A01 | 1 | 2 | 3 | 7 | 9 | 4 | 5 | 6 | 8 | 10 |
| A02 | 1 | 11 | 3 | 13 | 15 | 12 | 5 | 6 | 14 | 16 |
| A03 | 1 | 2 | 3 | 17 | 18 | 4 | 5 | 6 | 8 | 10 |
| A04 | 1 | 19 | 3 | 20 | 21 | 12 | 5 | 6 | 14 | 16 |
| A05 | 1 | 19 | 3 | 22 | 23 | 4 | 5 | 6 | 8 | 10 |
| A06 | 1 | 19 | 3 | 22 | 23 | 12 | 5 | 6 | 14 | 16 |
| A07 | 1 | 11 | 3 | 13 | 15 | 24 | 5 | 6 | 25 | 26 |
| A08 | 1 | 19 | 3 | 27 | 28 | 4 | 5 | 6 | 8 | 10 |
| A09 | 1 | 19 | 3 | 27 | 28 | 12 | 5 | 6 | 14 | 16 |
| A10 | 1 | 19 | 29 | 30 | 31 | 4 | 5 | 6 | 8 | 10 |
| A11 | 1 | 19 | 29 | 30 | 31 | 12 | 5 | 6 | 14 | 16 |
| A12 | 1 | 19 | 32 | 34 | 36 | 24 | 5 | 33 | 35 | 37 |
| A13 | 1 | 19 | 32 | 34 | 36 | 24 | 5 | 38 | 39 | 40 |
| A14 | 1 | 19 | 32 | 34 | 36 | 24 | 5 | 41 | 42 | 43 |
| A15 | 1 | 19 | 32 | 34 | 36 | 24 | 5 | 6 | 25 | 26 |
| A16 | 1 | 19 | 32 | 34 | 36 | 24 | 44 | 6 | 45 | 46 |
| A17 | 1 | 19 | 32 | 34 | 36 | 24 | 47 | 6 | 48 | 49 |
| A18 | 1 | 19 | 32 | 34 | 36 | 24 | 50 | 6 | 51 | 52 |
| A19 | 1 | 19 | 32 | 34 | 36 | 24 | 53 | 6 | 54 | 55 |
| A20 | 1 | 19 | 32 | 34 | 36 | 24 | 5 | 6 | 56 | 57 |
| A21 | 1 | 19 | 32 | 34 | 36 | 58 | 5 | 6 | 59 | 60 |
| A22 | 1 | 19 | 32 | 34 | 36 | 61 | 5 | 6 | 62 | 63 |
| A23 | 1 | 19 | 32 | 34 | 36 | 64 | 5 | 6 | 65 | 66 |
| A24 | 1 | 19 | 32 | 34 | 36 | 67 | 5 | 6 | 68 | 69 |
| A25 | 1 | 19 | 32 | 34 | 36 | 70 | 5 | 6 | 71 | 72 |
| A26 | 1 | 19 | 32 | 34 | 36 | 12 | 5 | 6 | 73 | 74 |
| A27 | 1 | 19 | 32 | 34 | 36 | 24 | 5 | 6 | 75 | 76 |
| A28 | 1 | 19 | 77 | 78 | 80 | 24 | 5 | 6 | 79 | 81 |
| A29 | 1 | 19 | 82 | 83 | 84 | 24 | 5 | 6 | 79 | 81 |
| A30 | 1 | 19 | 3 | 27 | 28 | 24 | 5 | 6 | 79 | 81 |
| A31 | 1 | 19 | 85 | 86 | 87 | 24 | 5 | 6 | 79 | 81 |
| A32 | 1 | 19 | 88 | 89 | 90 | 24 | 5 | 6 | 79 | 81 |
| A33 | 1 | 19 | 91 | 92 | 93 | 24 | 5 | 6 | 79 | 81 |
| A34 | 1 | 19 | 94 | 95 | 96 | 24 | 5 | 6 | 79 | 81 |
| A35 | 1 | 19 | 32 | 97 | 98 | 24 | 5 | 6 | 79 | 81 |
| A36 | 1 | 19 | 32 | 99 | 100 | 24 | 5 | 6 | 79 | 81 |
| A37 | 1 | 19 | 32 | 101 | 102 | 24 | 5 | 6 | 79 | 81 |
| A38 | 1 | 19 | 32 | 103 | 104 | 24 | 5 | 6 | 79 | 81 |
| A39 | 1 | 19 | 32 | 105 | 106 | 24 | 5 | 6 | 79 | 81 |
| A40 | 1 | 19 | 32 | 107 | 108 | 24 | 5 | 6 | 79 | 81 |
| A41 | 1 | 19 | 32 | 109 | 110 | 24 | 5 | 6 | 79 | 81 |
| A42 | 1 | 19 | 32 | 111 | 112 | 24 | 5 | 6 | 79 | 81 |
| A43 | 1 | 19 | 32 | 113 | 114 | 24 | 5 | 6 | 79 | 81 |
| A44 | 1 | 19 | 32 | 115 | 116 | 24 | 5 | 6 | 79 | 81 |
| A45 | 1 | 19 | 32 | 117 | 118 | 24 | 5 | 6 | 79 | 81 |
| A46 | 1 | 19 | 32 | 119 | 120 | 24 | 5 | 6 | 79 | 81 |
| A47 | 1 | 19 | 32 | 121 | 122 | 24 | 5 | 6 | 79 | 81 |
| A48 | 1 | 11 | 32 | 123 | 124 | 24 | 5 | 6 | 79 | 81 |
| A49 | 1 | 125 | 32 | 126 | 127 | 24 | 5 | 6 | 79 | 81 |
| A50 | 1 | 128 | 32 | 129 | 130 | 24 | 5 | 6 | 79 | 81 |
| A51 | 131 | 19 | 32 | 132 | 133 | 24 | 5 | 6 | 79 | 81 |
| A52 | 1 | 19 | 32 | 134 | 135 | 24 | 5 | 6 | 79 | 81 |
| A53 | 1 | 19 | 32 | 136 | 137 | 24 | 5 | 6 | 79 | 81 |
| A54 | 1 | 19 | 32 | 34 | 36 | 4 | 5 | 6 | 8 | 10 |
| A55 | 1 | 19 | 32 | 34 | 36 | 12 | 5 | 6 | 14 | 16 |
| A56 | 1 | 19 | 32 | 34 | 36 | 12 | 5 | 6 | 138 | 139 |
| A57 | 1 | 2 | 3 | 17 | 18 | 24 | 5 | 6 | 79 | 81 |
| A58 | 1 | 2 | 3 | 17 | 18 | 12 | 5 | 6 | 14 | 16 |
| A59 | 1 | 2 | 3 | 17 | 18 | 12 | 5 | 6 | 138 | 139 |
| A60 | 1 | 2 | 3 | 17 | 18 | 24 | 5 | 6 | 25 | 26 |
| A61 | 1 | 2 | 3 | 7 | 9 | 24 | 5 | 6 | 79 | 81 |
| A62 | 1 | 2 | 3 | 7 | 9 | 12 | 5 | 6 | 14 | 16 |
| A63 | 1 | 2 | 3 | 7 | 9 | 12 | 5 | 6 | 138 | 139 |
| A64 | 1 | 2 | 3 | 7 | 9 | 24 | 5 | 6 | 25 | 26 |
| A65 | 1 | 125 | 3 | 140 | 141 | 24 | 5 | 6 | 79 | 81 |
| A66 | 1 | 125 | 3 | 140 | 141 | 4 | 5 | 6 | 8 | 10 |
| A67 | 1 | 125 | 3 | 140 | 141 | 12 | 5 | 6 | 14 | 16 |
| A68 | 1 | 125 | 3 | 140 | 141 | 12 | 5 | 6 | 138 | 139 |
| A69 | 1 | 125 | 3 | 140 | 141 | 24 | 5 | 6 | 25 | 26 |
| A70 | 1 | 19 | 3 | 20 | 21 | 24 | 5 | 6 | 79 | 81 |
| A71 | 1 | 19 | 3 | 20 | 21 | 4 | 5 | 6 | 8 | 10 |
| A72 | 1 | 19 | 3 | 20 | 21 | 12 | 5 | 6 | 138 | 139 |
| A73 | 1 | 19 | 3 | 20 | 21 | 24 | 5 | 6 | 25 | 26 |
| A74 | 1 | 19 | 3 | 22 | 23 | 24 | 5 | 6 | 79 | 81 |
| A75 | 1 | 19 | 3 | 22 | 23 | 12 | 5 | 6 | 138 | 139 |
| A76 | 1 | 19 | 3 | 22 | 23 | 24 | 5 | 6 | 25 | 26 |
| A77 | 1 | 11 | 3 | 13 | 15 | 24 | 5 | 6 | 79 | 81 |
| A78 | 1 | 11 | 3 | 13 | 15 | 4 | 5 | 6 | 8 | 10 |
| A79 | 1 | 11 | 3 | 13 | 15 | 12 | 5 | 6 | 138 | 139 |
| A80 | 1 | 19 | 3 | 27 | 28 | 12 | 5 | 6 | 138 | 139 |
| A81 | 1 | 19 | 3 | 27 | 28 | 24 | 5 | 6 | 25 | 26 |
| A82 | 1 | 19 | 29 | 30 | 31 | 24 | 5 | 6 | 79 | 81 |
| A83 | 1 | 19 | 29 | 30 | 31 | 12 | 5 | 6 | 138 | 139 |
| A84 | 1 | 19 | 29 | 30 | 31 | 24 | 5 | 6 | 25 | 26 |
| A85 | 1 | 19 | 142 | 143 | 144 | 24 | 5 | 6 | 79 | 81 |
| A86 | 1 | 19 | 32 | 34 | 36 | 24 | 5 | 6 | 79 | 81 |
| A87 | 1 | 19 | 142 | 145 | 146 | 24 | 5 | 6 | 79 | 81 |
| A88 | 1 | 19 | 142 | 181 | 182 | 24 | 5 | 6 | 79 | 81 |
| A89 | 1 | 19 | 142 | 143 | 144 | 24 | 178 | 6 | 179 | 180 |
| A90 | 1 | 19 | 142 | 145 | 146 | 24 | 178 | 6 | 179 | 180 |
| A91 | 1 | 19 | 142 | 181 | 182 | 24 | 178 | 6 | 179 | 180 |
| A92 | 1 | 19 | 183 | 184 | 185 | 24 | 5 | 6 | 79 | 81 |
| A93 | 1 | 19 | 183 | 186 | 187 | 24 | 5 | 6 | 79 | 81 |
| A94 | 1 | 19 | 183 | 188 | 189 | 24 | 5 | 6 | 79 | 81 |
| A95 | 1 | 19 | 183 | 184 | 185 | 24 | 178 | 6 | 179 | 180 |
| A96 | 1 | 19 | 183 | 186 | 187 | 24 | 178 | 6 | 179 | 180 |
| A97 | 1 | 19 | 183 | 188 | 189 | 24 | 178 | 6 | 179 | 180 |
| A98 | 1 | 19 | 32 | 34 | 36 | 24 | 5 | 147 | 148 | 149 |
| A99 | 1 | 19 | 150 | 151 | 152 | 24 | 5 | 6 | 79 | 81 |
| A100 | 1 | 19 | 153 | 154 | 155 | 24 | 5 | 6 | 79 | 81 |
| A101 | 1 | 156 | 32 | 157 | 158 | 24 | 5 | 6 | 79 | 81 |
| A102 | 1 | 159 | 32 | 160 | 161 | 24 | 5 | 6 | 79 | 81 |
| A103 | 162 | 19 | 32 | 163 | 164 | 24 | 5 | 6 | 79 | 81 |
| A104 | 165 | 19 | 32 | 166 | 167 | 24 | 5 | 6 | 79 | 81 |
| P | 1 | 168 | 142 | 169 | — | 24 | 5 | 6 | 170 | — |

In some aspects, the present disclosure provides an antibody, or an antigen-binding fragment thereof, comprising any one of the sequences (SEQ ID NOs) described in Table 2 or a sequence variant thereof having at least 80% sequence identity (preferably at least 85%, more preferably at least 90%, even more preferably at least 95%, still more preferably at least 97%, particularly preferably at least 99% sequence identity). In some embodiments, such an antibody, or antigen-binding fragment thereof, comprises a set of six CDRs having the amino acid sequences of the six CDRs of any one of antibodies A1-A104 or P, as shown in Table 2, or in any one of Tables 3-8 below.

In some aspects, the present disclosure provides an anti-LAYN antibody, or an antigen-binding fragment thereof, comprising:
(a) a heavy chain variable region (VH) comprising:
   a heavy chain complementary determining region 1 (HCDR1) having the amino acid sequence of X₁YWMD (SEQ ID NO: 206), wherein
      X₁ is preferably S or G, more preferably S;
   a heavy chain complementary determining region 2 (HCDR2) having the amino acid sequence of NIX₂PX₃SGX₄TNYX₅X₆KFX₇X₈ (SEQ ID NO: 207), wherein
      X₂ is preferably Y or N, more preferably Y;
      X₃ is preferably S or G, more preferably S;
      X₄ is preferably T or S, more preferably T,
      X₅ is preferably A or D, more preferably A;
      X₆ is preferably Q or E, more preferably Q;
      X₇ is preferably Q or K, more preferably Q; and
      X₈ is preferably G or S, more preferably G; and
   a heavy chain complementary determining region 3 (HCDR3) having the amino acid sequence of X₉X₁₀YX₁₁RX₁₂AX₁₃X₁₄X₁₅ (SEQ ID NO: 208), wherein
      X₉ is preferably E or A, more preferably E;
      X₁₀ is preferably R, K or A, more preferably R;
      X₁₁ is preferably G or A, more preferably G;
      X₁₂ is preferably N, Q or A, more preferably N or A, even more preferably N;
      X₁₃ is preferably A or, more preferably A;
      X₁₄ is preferably E, D or A, more preferably E; and
      X₁₅ is preferably Y or A, more preferably Y; and
(b) a light chain variable region (VL) comprising
   a light chain complementary determining region 1 (LCDR1) having the amino acid sequence of X₁₆ASQX₁₇₁X₁₈X₁₉X₂₀LA (SEQ ID NO: 209), wherein
      X₁₆ is preferably R or L, more preferably R;
      X₁₇ is preferably G, T or S, more preferably G;
      X₁₈ is preferably G or S, more preferably G;
      X₁₉ is preferably S or T, more preferably S; and
      X₂₀ is preferably W or Y, more preferably W;
   a light chain complementary determining region 2 (LCDR2) having the amino acid sequence of AAX₂₁SLX₂₂X₂₃ (SEQ ID NO: 210), wherein
      X₂₁ is preferably T or S, more preferably T;
      X₂₂ is preferably A or Q, more preferably A; and
      X₂₃ is preferably D, T, S or E, more preferably D; and
   a light chain complementary determining region 3 (LCDR3) having the amino acid sequence of QQX₂₄YSX₂₅PLT (SEQ ID NO: 211), wherein
      X₂₄ is L, A or S, preferably L; and
      X₂₅ is preferably T or F, more preferably T.

Accordingly, the anti-LAYN antibody, or the antigen-binding fragment thereof, as described herein, may comprise a VH comprising a HCDR1, a HCDR2 and a HCDR3 having the consensus sequence according to SEQ ID NOs 206, 207 and 208, respectively, and a VL comprising a LCDR1, a LCDR2 and a LCDR3 having the consensus sequence according to SEQ ID NOs 209, 210 and 211, respectively.

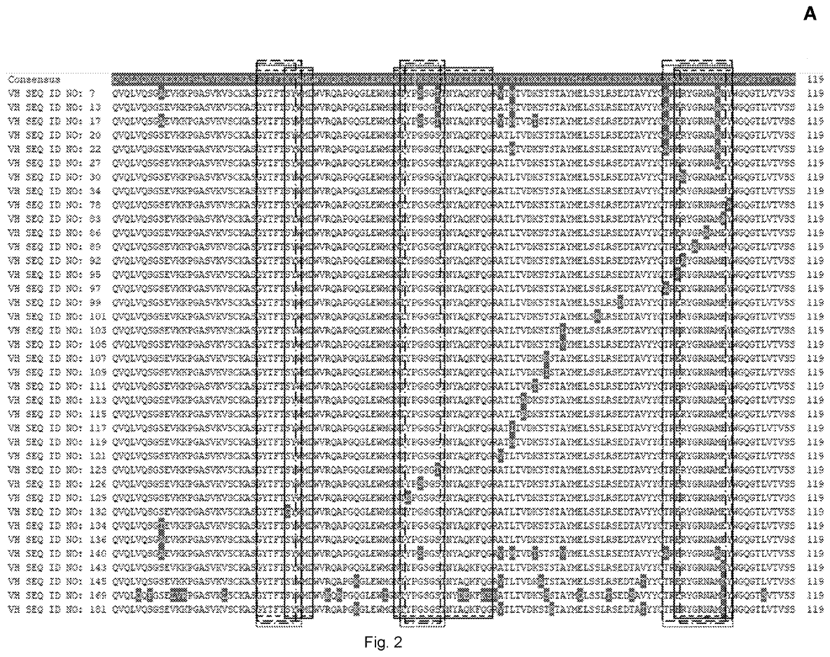

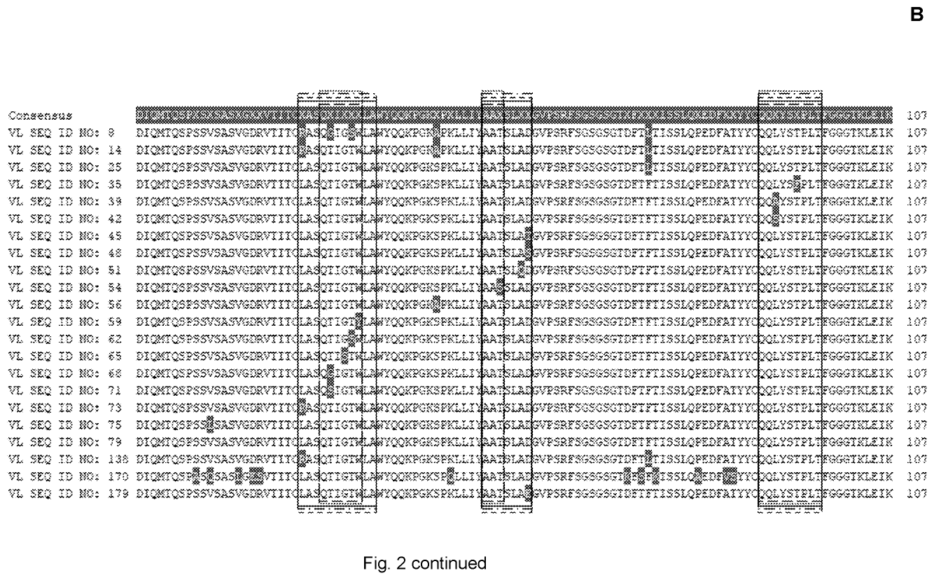

Example 3 herein below shows that exemplary antibodies bind to the ECD of human LAYN isoform 2 with high affinity. An alignment of the amino acid sequences of the VH and VL, respectively, of antibodies P and A1 to A88, A90 and A91 is shown in FIG. 2, wherein the CDR sequences (according to different numbering schemes) are shown in boxes. Thereby, the above consensus sequences were obtained.

Preferably, the anti-LAYN antibody, or the antigen-binding fragment thereof, as described herein, comprises:
(a) a VH comprising:
   a HCDR1 having the amino acid sequence of X₁YWMD (SEQ ID NO: 206), wherein
      X₁ is S or G;
   a HCDR2 having the amino acid sequence of NIX₂PX₃SGX₄TNYX₅X₆KFX₇X₈ (SEQ ID NO: 207), wherein
      X₂ is Y or N,
      X₃ is Sor G,
      X₄ is T or S,
      X₅ is A or D,
      X₆ is Q or E,
      X₇ is Q or K, and
      X₈ is G or S; and
   a HCDR3 having the amino acid sequence of X₉X₁₀YX₁₁RX₁₂AX₁₃X₁₄X₁₅ (SEQ ID NO: 208), wherein
      X₉ is E or A,
      X₁₀ is R, K or A,
      X₁₁ is G or A,
      X₁₂ is N or A,
      X₁₃ is A or M,
      X₁₄ is E, D or A, and
      X₁₅ is Y or A; and
(b) a VL comprising:
   a LCDR1 having the amino acid sequence of X₁₆ASQX₁₇₁X₁₈X₁₉X₂₀LA (SEQ ID NO: 209), wherein
      X₁₆ is R or L,
      X₁₇ is G, T or S,
      X₁₈ is G or S,
      X₁₉ is S or T, and
      X₂₀ is W or Y;
   a LCDR2 having the amino acid sequence of AAX₂₁SLX₂₂X₂₃ (SEQ ID NO: 210), wherein
      X₂₁ is T or S,
      X₂₂ is A or Q, and
      X₂₃ is D, T, S or E; and
   a LCDR3 having the amino acid sequence of QQX₂₄YSX₂₅PLT (SEQ ID NO: 211), wherein
      X₂₄ is L, A or S, and
      X₂₅ is T or F.

In some embodiments, the anti-LAYN antibody, or the antigen-binding fragment thereof, as described herein, comprises a heavy chain variable region comprising an HCDR1 having the amino acid sequence of any one of SEQ ID NOs 1 or 131, an HCDR2 having the amino acid sequence of any one of SEQ ID NOs 2, 11, 19, 125, 128 or 168, and an HCDR3 having the amino acid sequence of any one of SEQ ID NOs 3, 29, 32, 77, 82, 85, 88, 91, 94, 142 or 183, preferably an HCDR3 having the amino acid sequence of any one of SEQ ID NOS 3, 29, 32, 77, 82, 85, 88, 91, 94 or 142; and a light chain variable region comprising an LCDR1 having the amino acid sequence of any one of SEQ ID NOs 4, 12, 24, 58, 61, 64, 67 or 70, an LCDR2 having the amino acid sequence of any one of SEQ ID NOs 5, 44, 47, 50, 53 or 178, and an LCDR3 having the amino acid sequence of any one of SEQ ID NOs 6, 33, 38 or 41.

Preferably, the anti-LAYN antibody, or the antigen-binding fragment thereof, as described herein, comprises:
(i) a heavy chain variable region comprising an HCDR1 having the amino acid sequence of SEQ ID NO: 1, an HCDR2 having the amino acid sequence of SEQ ID NO: 2, and an HCDR3 having the amino acid sequence of SEQ ID NO: 3; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
(ii) a heavy chain variable region comprising an HCDR1 having the amino acid sequence of SEQ ID NO: 1, an HCDR2 having the amino acid sequence of SEQ ID NO: 11, and an HCDR3 having the amino acid sequence of SEQ ID NO: 3; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6; or
(iii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 3; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6; or
(iv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 3; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
(v) a heavy chain variable region comprising an HCDR1 having the amino acid sequence of SEQ ID NO: 1, an HCDR2 having the amino acid sequence of SEQ ID NO: 11, and an HCDR3 having the amino acid sequence of SEQ ID NO: 3; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(vi) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 29; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
(vii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 29; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
(viii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 29; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6; or
(ix) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 33; or 20
(x) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 38; or
(xi) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 41; or
(xii) a heavy chain variable region comprising an HCDR1 having the amino acid sequence of SEQ ID NO: 1, an HCDR2 having the amino acid sequence of SEQ ID NO: 19, and LCDR2 having the amino acid sequence of SEQ ID NO: 5, and an LCDR3 having the amino acid sequence of SEQ ID NO: 6; or
(xiii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 44, and an LCDR3 having the amino acid sequence of SEQ ID NO: 6; or
(xiv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 47, and an LCDR3 having the amino acid sequence of SEQ ID NO: 6; or
(xv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 50, and an LCDR3 having the amino acid sequence of SEQ ID NO: 6; or
(xvi) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 53, and an LCDR3 having the amino acid sequence of SEQ ID NO: 6; or
(xvii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 58, an LCDR2 having the amino acid sequence of SEQ ID NO: 5, and an LCDR3 having the amino acid sequence of SEQ ID NO: 6; or
(xviii) a heavy chain variable region comprising an HCDR1 having the amino acid sequence of SEQ ID NO: 1, an HCDR2 having the amino acid sequence of SEQ ID NO: 19, and region comprising an LCDR1 having the amino acid sequence of SEQ ID NO: 61, an LCDR2 having the amino acid sequence of SEQ ID NO: 5, and an LCDR3 having the amino acid sequence of SEQ ID NO: 6; or
(xix) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 64, an LCDR2 having the amino acid sequence of SEQ ID NO: 5, and an LCDR3 having the amino acid sequence of SEQ ID NO: 6; or
(xx) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 67, an LCDR2 having the amino acid sequence of SEQ ID NO: 5, and an LCDR3 having the amino acid sequence of SEQ ID NO: 6; or
(xxi) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 70, an LCDR2 having the amino acid sequence of SEQ ID NO: 5, and an LCDR3 having the amino acid sequence of SEQ ID NO: 6; or
(xxii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6; or
(xxiii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 77; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xxiv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 82; and a light chain variable LCDR2 having the amino acid sequence of SEQ ID NO: 5, and an LCDR3 having the amino acid sequence of SEQ ID NO: 6; or
(xxv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 3; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xxvi) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 85; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xxvii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 88; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xxviii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 91; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xxix) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 94; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xxx) a heavy chain variable region comprising an HCDR1 having the amino acid sequence of SEQ ID NO: 1, an HCDR2 having the amino acid sequence of SEQ ID NO: 11, and LCDR2 having the amino acid sequence of SEQ ID NO: 5, and an LCDR3 having the amino acid sequence of SEQ ID NO: 6; or
(xxxi) a heavy chain variable region comprising an HCDR1 having the amino acid sequence of SEQ ID NO: 1, an HCDR2 having the amino acid sequence of SEQ ID NO: 125, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xxxii) a heavy chain variable region comprising an HCDR1 having the amino acid sequence of SEQ ID NO: 1, an HCDR2 having the amino acid sequence of SEQ ID NO: 128, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xxxiii) a heavy chain variable region comprising an HCDR1 having the amino acid sequence of SEQ ID NO: 131, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xxxiv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
(xxxv) a heavy chain variable region comprising an HCDR1 having the amino acid sequence of SEQ ID NO: 1, an HCDR2 having the amino acid sequence of SEQ ID NO: 2, and an HCDR3 having the amino acid sequence of SEQ ID NO: 3; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xxxvi) a heavy chain variable region comprising an HCDR1 having the amino acid sequence of SEQ ID NO: 1, an HCDR2 having the amino acid sequence of SEQ ID NO: 2, and an HCDR3 having the amino acid sequence of SEQ ID NO: 3; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6; or
(xxxvii) a heavy chain variable region comprising an HCDR1 having the amino acid sequence of SEQ ID NO: 1, an HCDR2 having the amino acid sequence of SEQ ID NO: 125, and an HCDR3 having the amino acid sequence of SEQ ID NO: 3; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xxxviii) a heavy chain variable region comprising an HCDR1 having the amino acid sequence of SEQ ID NO: 1, an HCDR2 having the amino acid sequence of SEQ ID NO: 125, and an HCDR3 having the amino acid sequence of SEQ ID NO: 3; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
(xxxix) a heavy chain variable region comprising an HCDR1 having the amino acid sequence of SEQ ID NO: 1, an HCDR2 having the amino acid sequence of SEQ ID NO: 125, and an HCDR3 having the amino acid sequence of SEQ ID NO: 3; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6; or
(xl) a heavy chain variable region comprising an HCDR1 having the amino acid sequence of SEQ ID NO: 1, an HCDR2 having the amino acid sequence of SEQ ID NO: 11, and an HCDR3 having the amino acid sequence of SEQ ID NO: 3; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
(xli) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 29; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xlii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 142; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xliii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 142; and a light chain variable region comprising an LCDR1 having the amino acid sequence of SEQ ID NO: 24, an LCDR2 having the amino acid sequence of SEQ ID NO: 178, and an LCDR3 having the amino acid sequence of SEQ ID NO: 6; or
(xliv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 183; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(xlv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 183; and a light chain variable region comprising an LCDR1 having the amino acid sequence of SEQ ID NO: 24, an LCDR2 having the amino acid sequence of SEQ ID NO: 178, and an LCDR3 having the amino acid sequence of SEQ ID NO: 6; or
(xlvi) a heavy chain variable region comprising an HCDR1 having the amino acid sequence of SEQ ID NO: 1, an HCDR2 having the amino acid sequence of SEQ ID NO: 168, and an HCDR3 having the amino acid sequence of SEQ ID NO: 142; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6.

While the above-described CDR sequences (incl. Table 2) are according to the numbering scheme of Kabat, the skilled person can readily determine the corresponding CDR sequences according to other numbering schemes, in particular based on the VH and VL sequences in Table 2. For example, the CDR sequences of the exemplary antibodies according to the numbering schemes of Kabat, Aho (Honegger and Plückthun, 2001, Journal of Molecular Biology 309 (3): 657-670, doi.org/10.1006/jmbi.2001.4662), CCG (Chemical Computing Group; as illustrated in Almagro et al., Proteins 2011; 79:3050-3066 and Maier et al, Proteins 2014; 82:1599-1610), Chothia (Chothia and Lesk, 1987, J. Mol. Biol. 196:901-917) and IMGT (Lefranc, 2003, Developmental and Comparative Immunology 27:55-77) are shown in FIG. 2 and in Tables 3 to 8 below.

TABLE 3
SEQ ID NOs of HCDR1 sequences of exemplary antibodies
according to Kabat, AHo, CCG, Chothia and IMGT

| Antibodies | Kabat | AHo | CCG | Chothia | IMGT |
|---|---|---|---|---|---|
| A1-A50, A52-A102, P | 1 | 230 | 226 | 233 | 230 |
| A51 | 131 | 231 | 227 | 234 | 231 |
| A103 | 162 | 230 | 228 | 233 | 230 |
| A104 | 165 | 232 | 229 | 233 | 232 |

TABLE 4
SEQ ID NOs of HCDR2 sequences of exemplary antibodies
according to Kabat, AHo, CCG, Chothia and IMGT

| Antibodies | Kabat | AHo | CCG | Chothia | IMGT |
|---|---|---|---|---|---|
| A1, A3, A57-A64 | 2 | 235 | 2 | 240 | 235 |
| A2, A7, A48, A77-A79 | 11 | 236 | 11 | 241 | 236 |
| A4-A6, A8-A47, A51-A56, A70- | 19 | 237 | 19 | 242 | 237 |
| A76, A80-A100, A103, A104 | | | | | |
| A49, A65-A69 | 125 | 238 | 125 | 243 | 238 |
| A50 | 128 | 239 | 128 | 244 | 239 |
| A101 | 156 | 237 | 156 | 242 | 237 |
| A102 | 159 | 237 | 159 | 242 | 237 |
| P | 168 | 237 | 168 | 242 | 237 |

TABLE 5
SEQ ID NOs of HCDR3 sequences of exemplary antibodies
according to Kabat, AHo, CCG, Chothia and IMGT

| Antibodies | Kabat | AHo | CCG | Chothia | IMGT |
|---|---|---|---|---|---|
| A1-A7, A57-A79 | 3 | 245 | 3 | 260 | 245 |
| A8, A9, A30, A80, A81 | 3 | 246 | 3 | 260 | 246 |
| A10, A11, A82-A84 | 29 | 247 | 29 | 261 | 247 |
| A12-A27, A36-A56, A86, | 32 | 248 | 32 | 262 | 248 |
| A98, A101-A104 | | | | | |
| A28 | 77 | 249 | 77 | 262 | 249 |
| A29 | 82 | 250 | 82 | 263 | 250 |
| A31 | 85 | 251 | 85 | 264 | 251 |
| A32 | 88 | 252 | 88 | 265 | 252 |
| A33 | 91 | 253 | 91 | 266 | 253 |
| A34 | 94 | 254 | 94 | 262 | 254 |
| A35 | 32 | 255 | 32 | 262 | 255 |
| A85, A87-A91, P | 142 | 256 | 142 | 267 | 256 |
| A92-A97 | 183 | 257 | 183 | 268 | 257 |
| A99 | 150 | 258 | 150 | 269 | 258 |
| A100 | 153 | 259 | 153 | 270 | 259 |

TABLE 6
SEQ ID NOs of LCDR1 sequences of exemplary antibodies
according to Kabat, AHo, CCG, Chothia and IMGT

| Antibodies | Kabat | AHo | CCG | Chothia | IMGT |
|---|---|---|---|---|---|
| A1, A3, A5, A8, A10, A54, A66, | 4 | 271 | 4 | 4 | 271 |
| A71, A78 | | | | | |
| A2, A4, A6, A9, A11, A26, A55, | 12 | 272 | 12 | 12 | 272 |
| A56, A58, A59, A62, A63, A67, | | | | | |
| A68, A72, A75, A79, A80, A83 | | | | | |
| A7, A12-A20, A27-A53, A57, A60, | 24 | 272 | 24 | 24 | 272 |
| A61, A64, A65, A69, A70, A73, | | | | | |
| A74, A76, A77, A81, A82, A84- | | | | | |
| A104, P | | | | | |
| A21 | 58 | 214 | 58 | 58 | 214 |
| A22 | 61 | 215 | 61 | 61 | 215 |
| A23 | 64 | 216 | 64 | 64 | 216 |
| A24 | 67 | 217 | 67 | 67 | 217 |
| A25 | 70 | 218 | 70 | 70 | 218 |

TABLE 7
SEQ ID NOs of LCDR2 sequences of exemplary antibodies
according to Kabat, AHo, CCG, Chothia and IMGT

| Antibodies | Kabat | AHo* | CCG | Chothia | IMGT* |
|---|---|---|---|---|---|
| A1-A15, A20-A88, A92-A94, | 5 | AAT | 5 | 5 | AAT |
| A98-A104, P | | | | | |
| A16 | 44 | AAT | 44 | 44 | AAT |
| A17 | 47 | AAT | 47 | 47 | AAT |
| A18 | 50 | AAT | 50 | 50 | AAT |
| A19 | 53 | AAS | 53 | 53 | AAS |
| A89-A91, A95-A97 | 178 | AAT | 178 | 178 | AAT |

*AHo and IMGT sequences shown as
actual amino acid sequence in one-letter code
(AAT and AAS, respectively),
no SEQ ID NOs assigned in these cases

TABLE 8
SEQ ID NOs of LCDR3 sequences of exemplary antibodies
according to Kabat, Aho, CCG, Chothia and IMGT

| Antibodies | Kabat | Aho | CCG | Chothia | IMGT |
|---|---|---|---|---|---|
| A1-A11, A15-A97, A99-A104, P | 6 | 6 | 6 | 6 | 6 |
| A12 | 33 | 33 | 33 | 33 | 33 |
| A13 | 38 | 38 | 38 | 38 | 38 |
| A14 | 41 | 41 | 41 | 41 | 41 |
| A98 | 147 | 147 | 147 | 147 | 147 |

It is understood that the present disclosure comprises anti-LAYN antibodies, wherein the CDR sequences may be determined according to any of the CDR numbering schemes, e.g. according to Kabat, Aho, CCG, Chothia and IMGT described herein.

### Amino Acid Sequence Variants

Variant anti-LAYN antibodies and antibody fragments thereof can be engineered based on a set of CDRs depicted in Tables 2 to 8. It is to be understood that in the variant anti-LAYN antibodies and antibody fragments the amino acid sequence of the CDRs remain unchanged or have minimal changes (e.g., no more than 1, 2, 3, 4 or 5 changes), but the surrounding regions, e.g., FR regions can be engineered. Amino acid sequence variants of the anti-LAYN antibody can be prepared by introducing appropriate nucleotide changes into the anti-LAYN antibody DNA, or by peptide synthesis. Such variants include, for example, deletions from, and/or insertions into and/or substitutions of, residues within the amino acid sequences of the exemplary anti-LAYN antibodies (as shown in Tables 2-8). Any combination of deletions, insertions, and substitutions is made to arrive at the final construct, provided that the final construct possesses the desired characteristics. The amino acid changes also may alter post-translational processes of the human or variant anti-LAYN antibody, such as changing the number or position of glycosylation sites.

In some aspects, the present disclosure provides an anti-LAYN antibody, or an antigen-binding fragment thereof, comprising:
a heavy chain variable region (VH) comprising the amino acid sequence of QVQLX₂₆QX₂₇GX₂₈EX₂₉X₃₀X₃₁PGASVKX₃₂SCKASGYTFTX₁YWMDWVX₃₃QX₃₄PGX₃₅GL EWX₃₆GNIX₂PX₃SGX₄TNYX₅X₆KFX₇X₈X₃₇X₃₈TX₃₉TX₄₀DX₄₁X₄₂X₄₃X₄₄TX₄₅YMX₄₆LSX₄₇LX 48SX₄₉DX₅₀AX₅₁YYCX₅₂RX₉X₁₀YX₁₁RX₁₂AX₁₃X₁₄X₁₅WGQGTX₅₃VTVSS
(SEQ ID NO: 212), wherein
   X₁ is preferably S or G, more preferably S,
   X₂ is preferably Y or N, more preferably Y,
   X₃ is preferably G or S, more preferably S,
   X₄ is preferably S or T, more preferably T,
   X₅ is preferably D or A, more preferably A,
   X₆ is preferably E or Q, more preferably Q,
   X₇ is preferably K or Q, more preferably Q,
   X₈ is preferably S or G, more preferably G,
   X₉ is preferably E or A, more preferably E,
   X₁₀ is preferably R, K or A, more preferably R,
   X₁₁ is preferably G or A, more preferably G,
   X₁₂ is preferably N, Q or A, more preferably N or A, even more preferably N,
   X₁₃ is preferably M or A, more preferably A,
   X₁₄ is preferably D, E or A, more preferably E,
   X₁₅ is preferably Y or A, more preferably Y,
   X₂₆ is preferably V or Q, more preferably V,
   X₂₇ is preferably S or P, more preferably S,
   X₂₈ is preferably A, S or T, more preferably A,
   X₂₉ is preferably V or L, more preferably V,
   X₃₀ is preferably K or V, more preferably K,
   X₃₁ is preferably K or R, more preferably K,
   X₃₂ is preferably V or L, more preferably V,
   X₃₃ is preferably R or K, more preferably R,
   X₃₄ is preferably A or R, more preferably A,
   X₃₅ is preferably Q or K, more preferably Q,
   X₃₆ is preferably M or I, more preferably M,
   X₃₇ is preferably R or K, more preferably R,
   X₃₈ is preferably V or A, more preferably V,
   X₃₉ is preferably M, L or I, more preferably M,
   X₄₀ is preferably V, A or R, more preferably V,
   X₄₁ is preferably K or T, more preferably K,
   X₄₂ is preferably S or A, more preferably S,
   X₄₃ is preferably T, K, I or S, more preferably T,
   X₄₄ is preferably S or D, more preferably S,
   X₄₅ is preferably A, L or V, more preferably A,
   X₄₆ is preferably E or Q, more preferably E,
   X₄₇ is preferably S or R, more preferably S,
   X₄₈ is preferably R or T, more preferably R,
   X₄₉ is preferably E or D, more preferably E,
   X₅₀ is preferably T or S, more preferably T,
   X₅₁ is preferably V or T, more preferably V,
   X₅₂ is preferably A or T, more preferably A, and
   X₅₃ is preferably L or S, more preferably L; and
a light chain variable region (VL) comprising the amino acid sequence of DIQMTQSPX₅₄SX₅₅SASX₅₆GX₅₇X₅₈VTITCX₁₆ASQX₁₇₁X₁₈X₁₉X₂₀LAWYQQKPGKX₅₉PX₆₀L LIYAAX₂₁SLX₂₂X₂₃GVPSRFSGSGSGTX₆₁FX₆₂X₆₃X₆₄ISSLQX₆₅EDFX₆₆X₆₇YYCQQX₂₄YS X₂₅PLTFGGGTKLEIK
(SEQ ID NO: 213), wherein
   X₁₆ is preferably L or R, more preferably R,
   X₁₇ is preferably T, G or S, more preferably G,
   X₁₈ is preferably G or S, more preferably G,
   X₁₉ is preferably T or S, more preferably S,
   X₂₀ is preferably W or Y, more preferably W,
   X₂₁ is preferably T or S, more preferably T,
   X₂₂ is preferably A or Q, more preferably A,
   X₂₃ is preferably D, T, S or E, more preferably D,
   X₂₄ is L, A or S, preferably L,
   X₂₅ is preferably T or F, more preferably T,
   X₅₄ is preferably S or A, more preferably S,
   X₅₅ is preferably V, L or Q, more preferably V,
   X₅₆ is preferably V or L, more preferably V,
   X₅₇ is preferably D or E, more preferably D,
   X₅₈ is preferably R or S, more preferably R,
   X₅₉ is preferably A or S, more preferably A,
   X₆₀ is preferably K or Q, more preferably K,
   X₆₁ is preferably D or K, more preferably D,
   X₆₂ is preferably T or S, more preferably T,
   X₆₃ is preferably L or F, more preferably L,
   X₆₄ is preferably T or K, more preferably T,
   X₆₅ is preferably P or A, more preferably P,
   X₆₆ is preferably A or V, more preferably A, and
   X₆₇ is preferably T or S, more preferably T.

Accordingly, the anti-LAYN antibody, or the antigen-binding fragment thereof, as described herein, may comprise a VH having the consensus sequence according to SEQ ID NO: 212 and a VL having the consensus sequence according to SEQ ID NO: 213. Similarly as the above-described consensus CDR sequences, the consensus VH and VL sequences were obtained based on an alignment of the amino acid sequences of the VH and VL, respectively, of antibodies P and A1 to A88, A90 and A91, as shown in FIG. 2.

Preferably, the anti-LAYN antibody, or the antigen-binding fragment thereof, as described herein, comprises:
a VH having the consensus sequence according to SEQ ID NO: 212, wherein
   X₁ is S or G,
   X₂ is Y or N,
   X₃ is G or S,
   X₄ is S or T,
   X₅ is D or A,
   X₆ is E or Q,
   X₇ is K or Q,
   X₈ is Sor G,
   X₉ is E or A,
   X₁₀ is R, K or A,
   X₁₁ is G or A,
   X₁₂ is N or A,
   X₁₃ is M or A,
   X₁₄ is D, E or A,
   X₁₅ is Y or A,
   X₂₆ is V or Q,
   X₂₇ is S or P,
   X₂₈ is A, S or T,
   X₂₉ is V or L,
   X₃₀ is K or V,
   X₃₁ is Kor R,
   X₃₂ is V or L,
   X₃₃ is R or K,
   X₃₄ is A or R,
   X₃₅ is Q or K,
   X₃₆ is M or I,
   X₃₇ is R or K,
   X₃₈ is V or A,
   X₃₉ is M, L or I,
   X₄₀ is V, A or R,
   X₄₁ is K or T,
   X₄₂ is S or A,
   X₄₃ is T, K, I or S,
   X₄₄ is S or D,
   X₄₅ is A, L or V,
   X₄₆ is E or Q,
   X₄₇ is S or R,
   X₄₈ is R or T,
   X₄₉ is E or D,
   X₅₀ is T or S,
   X₅₁ is V or T,
   X₅₂ is A or T, and
   X₅₃ is L or S; and
a VL having the consensus sequence according to SEQ ID NO: 213, wherein
   X₁₆ is L or R,
   X₁₇ is T, G or S,
   X₁₈ is G or S,
   X₁₉ is T or S,
   X₂₀ is W or Y,
   X₂₁ is T or S,
   X₂₂ is A or Q,
   X₂₃ is D, T, S or E,
   X₂₄ is L, A or S,
   X₂₅ is T or F,
   X₅₄ is S or A,
   X₅₅ is V, L or Q,
   X₅₆ is V or L,
   X₅₇ is D or E,
   X₅₈ is R or S,
   X₅₉ is A or S,
   X₆₀ is K or Q,
   X₆₁ is D or K,
   X₆₂ is T or S,
   X₆₃ is L or F,
   X₆₄ is T or K,
   X₆₅ is P or A,
   X₆₆ is A or V, and
   X₆₇ is T or S.

Preferably, in the consensus VH and VL sequences, the specific set of CDRs, as described for the exemplary antibodies in Table 2, is maintained. In other words, the anti-LAYN antibody, or the antigen-binding fragment thereof, as described herein, preferably comprises a VH having the consensus sequence according to SEQ ID NO: 212 and a VL having the consensus sequence according to SEQ ID NO: 213, wherein the specific set of the six CDR sequences (HCDR1, HCDR2, HCDR3, LCDR1, LCDR2 and LCDR3) of any one of antibodies A1-A97 and P (as described in Tables 2-8), preferably of antibodies A1-A88, A90 and A91, are maintained.

In some embodiments, the anti-LAYN antibody, or the antigen-binding fragment thereof, as described herein, comprises a VH having the consensus sequence according to SEQ ID NO: 212 and a VL having the consensus sequence according to SEQ ID NO: 213, wherein the VH comprises an HCDR1 having the amino acid sequence of SEQ ID NO: 1, an HCDR2 having the amino acid sequence of SEQ ID NO: 2, and an HCDR3 having the amino acid sequence of SEQ ID NO: 3; and wherein the VL comprises an LCDR1 having the amino acid sequence of SEQ ID NO: 4, an LCDR2 having the amino acid sequence of SEQ ID NO: 5, and an LCDR3 having the amino acid sequence of SEQ ID NO: 6.

In some embodiments, the anti-LAYN antibody, or the antigen-binding fragment thereof, as described herein, comprises a VH having the consensus sequence according to SEQ ID NO: 212 and a VL having the consensus sequence according to SEQ ID NO: 213, wherein the VH comprises an HCDR1 having the amino acid sequence of SEQ ID NO: 1, an HCDR2 having the amino acid sequence of SEQ ID NO: 11, and an HCDR3 having the amino acid sequence of SEQ ID NO: 3; and wherein the VL comprises an LCDR1 having the amino acid sequence of SEQ ID NO: 12, an LCDR2 having the amino acid sequence of SEQ ID NO: 5, and an LCDR3 having the amino acid sequence of SEQ ID NO: 6.

In some embodiments, the anti-LAYN antibody, or the antigen-binding fragment thereof, as described herein, comprises a VH having the consensus sequence according to SEQ ID NO: 212 and a VL having the consensus sequence according to SEQ ID NO: 213, wherein the VH comprises an HCDR1 having the amino acid sequence of SEQ ID NO: 1, an HCDR2 having the amino acid sequence of SEQ ID NO: 2, and an HCDR3 having the amino acid sequence of SEQ ID NO: 3; and wherein the VL comprises an LCDR1 having the amino acid sequence of SEQ ID NO: 4, an LCDR2 having the amino acid sequence of SEQ ID NO: 5, and an LCDR3 having the amino acid sequence of SEQ ID NO: 6.

In some embodiments, the anti-LAYN antibody, or the antigen-binding fragment thereof, as described herein, comprises a VH having the consensus sequence according to SEQ ID NO: 212 and a VL having the consensus sequence according to SEQ ID NO: 213, wherein the VH comprises an HCDR1 having the amino acid sequence of SEQ ID NO: 1, an HCDR2 having the amino acid sequence of SEQ ID NO: 19, and an HCDR3 having the amino acid sequence of SEQ ID NO: 142; and wherein the VL comprises an LCDR1 having the amino acid sequence of SEQ ID NO: 24, an LCDR2 having the amino acid sequence of SEQ ID NO: 5, and an LCDR3 having the amino acid sequence of SEQ ID NO: 6.

In some embodiments, the anti-LAYN antibody, or the antigen-binding fragment thereof, as described herein, comprises a VH having the consensus sequence according to SEQ ID NO: 212 and a VL having the consensus sequence according to SEQ ID NO: 213, wherein the VH comprises an HCDR1 having the amino acid sequence of SEQ ID NO: 1, an HCDR2 having the amino acid sequence of SEQ ID NO: 19, and an HCDR3 having the amino acid sequence of SEQ ID NO: 32; and wherein the VL comprises an LCDR1 having the amino acid sequence of SEQ ID NO: 24, an LCDR2 having the amino acid sequence of SEQ ID NO: 5, and an LCDR3 having the amino acid sequence of SEQ ID NO: 6.

In some aspects, the present disclosure provides an anti-LAYN antibody, or an antigen-binding fragment thereof, comprising a heavy chain variable region comprising an amino acid sequence at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188, preferably a heavy chain variable region comprising an amino acid sequence at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181; and a light chain variable region comprising an amino acid sequence at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179. In embodiments, said anti-LAYN antibody, or an antigen-binding fragment thereof, binds to full length hLAYN isoform 2 (e.g., having the amino acid sequence of SEQ ID NO: 194) with a high binding affinity. In embodiments, high affinity as described herein includes wherein the K_{D} value is between about 0.001 nM and 50 nM, between about 0.01 nM and 50 nM, between about 0.1 nM and 50 nM, between about 1 nM and 50 nM, or less than about 50 nM. In embodiments, said anti-LAYN antibody, or an antigen-binding fragment thereof, has a high stability. In embodiments, high stability as described herein includes wherein the melting temperature or an inflection point (IP) of a thermal transition curve of the anti-LAYN antibody, or an antigen-binding fragment thereof, is between about 80 °C and 99 °C, between about 80 °C and 95 °C, between about 80 °C and 90 °C, or between about 80 °C and 85 °C, or greater than 80 °C.

Accordingly, the anti-LAYN antibody, or the antigen-binding fragment thereof, as described herein, may comprise a heavy chain variable region comprising an amino acid sequence at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188, preferably a heavy chain variable region comprising an amino acid sequence at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181; and a light chain variable region comprising an amino acid sequence at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179.

As noted above, it is to be understood that in the variant anti-LAYN antibodies and antibody fragments the amino acid sequence of the CDRs remain unchanged or have minimal changes (e.g., no more than 1, 2, 3, 4 or 5 changes), but the surrounding regions, e.g., FR regions can be engineered.

Preferably, the variant anti-LAYN antibodies and antigen-binding fragments include the specific sets of six CDRs of antibodies A1 to A97 and P, e.g., as described in Tables 2-8 (without any changes to the CDR sequences), preferably of antibodies A1-A88, A90 and A91. Accordingly, the %-sequence identity indicated for the entire VH or VL sequence corresponds preferably to the %-sequence identity of all four FRs (FR1, FR2, FR3 and FR4 taken together) of the respective VH or VL sequence.

Preferably, the anti-LAYN antibody, or the antigen-binding fragment thereof, as described herein, comprises:
(i) a VH comprising an amino acid sequence at least 80% identical to a VH amino acid sequence of any one of antibodies A1 to A97 and P (e.g., as disclosed in Table 2), preferably of antibodies A1-A88, A90 and A91; and
(ii) a VL comprising an amino acid sequence at least 80% identical to the corresponding VL amino acid sequence of the antibody selected in (i) (out of antibodies A1 to A97 and P (e.g., as disclosed in Table 2), preferably of antibodies A1-A88, A90 and A91).

In some embodiments, the anti-LAYN antibody, or the antigen-binding fragment thereof, as described herein, comprises a heavy chain variable region comprising an amino acid sequence at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188, preferably a heavy chain variable region comprising an amino acid sequence at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181; and a light chain variable region comprising an amino acid sequence at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179. In embodiments, said anti-LAYN antibody, or an antigen-binding fragment thereof, binds to full length hLAYN isoform 2 (e.g., having the amino acid sequence of SEQ ID NO: 194) with a high binding affinity. In embodiments, high affinity as described herein includes wherein the K_{D} value is between about 0.001 nM and 50 nM, between about 0.01 nM and 50 nM, between about 0.1 nM and 50 nM, between about 1 nM and 50 nM, or less than about 50 nM. In embodiments, said anti-LAYN antibody, or an antigen-binding fragment thereof, has a high stability. In embodiments, high stability as described herein includes wherein the melting temperature or an inflection point (IP) of a thermal transition curve of the anti-LAYN antibody, or an antigen-binding fragment thereof, is between about 80 °C and 99 °C, between about 80 °C and 95 °C, between about 80 °C and 90 °C, or between about 80 °C and 85 °C, or greater than 80 °C.

More preferably, the anti-LAYN antibody, or the antigen-binding fragment thereof, as described herein, comprises:
(i) a VH comprising an amino acid sequence at least 85% identical to a VH amino acid sequence of any one of antibodies A1 to A97 and P (e.g., as disclosed in Table 2), preferably of antibodies A1-A88, A90 and A91; and
(ii) a VL comprising an amino acid sequence at least 85% identical to the corresponding VL amino acid sequence of the antibody selected in (i) (out of antibodies A1 to A97 and P (e.g., as disclosed in Table 2), preferably of antibodies A1-A88, A90 and A91).

In some embodiments, the anti-LAYN antibody, or the antigen-binding fragment thereof, as described herein, comprises a heavy chain variable region comprising an amino acid sequence at least 90% (e.g., at least 91%, at least 92%, at least 93%, at least 94%, at least 95%, at least 96%, at least 97%, at least 98%, at least 99% or 100%) identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188, preferably a heavy chain variable region comprising an amino acid sequence at least 90% (e.g., at least 91%, at least 92%, at least 93%, at least 94%, at least 95%, at least 96%, at least 97%, at least 98%, at least 99% or 100%) identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181; and a light chain variable region comprising an amino acid sequence at least 90% (e.g., at least 91%, at least 92%, at least 93%, at least 94%, at least 95%, at least 96%, at least 97%, at least 98%, at least 99% or 100%) identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179. In embodiments, said anti-LAYN antibody, or an antigen-binding fragment thereof, binds to full length hLAYN isoform 2 (e.g., having the amino acid sequence of SEQ ID NO: 194) with a high binding affinity. In embodiments, high affinity as described herein includes wherein the K_{D} value is between about 0.001 nM and 50 nM, between about 0.01 nM and 50 nM, between about 0.1 nM and 50 nM, between about 1 nM and 50 nM, or less than about 50 nM. In embodiments, said anti-LAYN antibody, or an antigen-binding fragment thereof, has a high stability. In embodiments, high stability as described herein includes wherein the melting temperature or an inflection point (IP) of a thermal transition curve of the anti-LAYN antibody, or an antigen-binding fragment thereof, is between about 80 °C and 99 °C, between about 80 °C and 95 °C, between about 80 °C and 90 °C, or between about 80 °C and 85 °C, or greater than 80 °C.

Even more preferably, the anti-LAYN antibody, or the antigen-binding fragment thereof, as described herein, comprises:
(i) a VH comprising an amino acid sequence at least 90%, preferably at least 92.5%, more preferably at least 95%, still more preferably at least 97%, even more preferably at least 98% and particularly preferably at least 99% identical to a VH amino acid sequence of any one of antibodies A1 to A97 and P (e.g., as disclosed in Table 2), preferably of antibodies A1-A88, A90 and A91; and
(ii) a VL comprising an amino acid sequence at least 90%, preferably at least 92.5%, more preferably at least 95%, still more preferably at least 97%, even more preferably at least 98% and particularly preferably at least 99% identical to the corresponding VL amino acid sequence of the antibody selected in (i) of antibodies A1 to A97 and P (e.g., as disclosed in Table 2), preferably of antibodies A1-A88, A90 and A91.

In some embodiments, the anti-LAYN antibody, or the antigen-binding fragment thereof, as described herein, comprises a heavy chain variable region comprising an amino acid sequence at least 95% (e.g., at least 96%, at least 97%, at least 98%, at least 99% or 100%) identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188, preferably a heavy chain variable region comprising an amino acid sequence at least 95% (e.g., at least 96%, at least 97%, at least 98%, at least 99% or 100%) identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181; and a light chain variable region comprising an amino acid sequence at least 95% (e.g., at least 96%, at least 97%, at least 98%, at least 99% or 100%) identical to the amino acid sequence of any one of SEQ ID NOs 8, 14, 25, 35, 39, 42, 45, 48, 51, 54, 56, 59, 62, 65, 68, 71, 73, 75, 79, 138, 170 or 179. In embodiments, said anti-LAYN antibody, or an antigen-binding fragment thereof, binds to full length hLAYN isoform 2 (e.g., having the amino acid sequence of SEQ ID NO: 194) with a high binding affinity. In embodiments, high affinity as described herein includes wherein the K_{D} value is between about 0.001 nM and 50 nM, between about 0.01 nM and 50 nM, between about 0.1 nM and 50 nM, between about 1 nM and 50 nM, or less than about 50 nM. In embodiments, said anti-LAYN antibody, or an antigen-binding fragment thereof, has a high stability. In embodiments, high stability as described herein includes wherein the melting temperature or an inflection point (IP) of a thermal transition curve of the anti-LAYN antibody, or an antigen-binding fragment thereof, is between about 80 °C and 99 °C, between about 80 °C and 95 °C, between about 80 °C and 90 °C, or between about 80 °C and 85 °C, or greater than 80 °C.

Still more preferably, the anti-LAYN antibody, or the antigen-binding fragment thereof, as described herein, comprises:
(i) a VH comprising an amino acid sequence at least 95% identical to a VH amino acid sequence of any one of antibodies A1 to A97 and P (e.g., as disclosed in Table 2), preferably of antibodies A1-A88, A90 and A91; and
(ii) a VL comprising an amino acid sequence at least 95% identical to the corresponding VL amino acid sequence of the antibody selected in (i) (out of antibodies A1 to A97 and P (e.g., as disclosed in Table 2), preferably of antibodies A1-A88, A90 and A91).

In some embodiments, the anti-LAYN antibody, or the antigen-binding fragment thereof, as described herein, comprises a heavy chain variable region comprising an amino acid sequence at least 98% (e.g., at least 99% or 100%) identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188, preferably a heavy chain variable region comprising an amino acid sequence at least 98% (e.g., at least 99% or 100%) identical to the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181; and a light chain variable region comprising an amino acid sequence at least 98% (e.g., at least 99% or 100%) identical to the amino acid sequence of any one of SEQ ID NOS 8, 14, 25, 35, 39, 42, 45, 48, 51, 54, 56, 59, 62, 65, 68, 71, 73, 75, 79, 138, 170 or 179. In embodiments, said anti-LAYN antibody, or an antigen-binding fragment thereof, binds to full length hLAYN isoform 2 (e.g., having the amino acid sequence of SEQ ID NO: 194) with a high binding affinity. In embodiments, high affinity as described herein includes wherein the K_{D} value is between about 0.001 nM and 50 nM, between about 0.01 nM and 50 nM, between about 0.1 nM and 50 nM, between about 1 nM and 50 nM, or less than about 50 nM. In embodiments, said anti-LAYN antibody, or an antigen-binding fragment thereof, has a high stability. In embodiments, high stability as described herein includes wherein the melting temperature or an inflection point (IP) of a thermal transition curve of the anti-LAYN antibody, or an antigen-binding fragment thereof, is between about 80 °C and 99 °C, between about 80 °C and 95 °C, between about 80 °C and 90 °C, or between about 80 °C and 85 °C, or greater than 80 °C.

Particularly preferably, the anti-LAYN antibody, or the antigen-binding fragment thereof, as described herein, comprises:
(i) a VH comprising an amino acid sequence at least 98% identical to a VH amino acid sequence of any one of antibodies A1 to A97 and P (e.g., as disclosed in Table 2), preferably of antibodies A1-A88, A90 and A91; and
(ii) a VL comprising an amino acid sequence at least 98% identical to the corresponding VL amino acid sequence of the antibody selected in (i) (out of antibodies A1 to A97 and P (e.g., as disclosed in Table 2), preferably of antibodies A1-A88, A90 and A91).

In some embodiments, the present invention includes anti-LAYN antibodies having a heavy chain and a light chain, wherein the heavy chain amino acid sequence and the light chain amino acid sequence are at least 95%, preferably at least 98%, or more preferably at least 99% identical to the amino acid sequences disclosed in Table 2 provided that the antibody or fragments thereof retain binding to LAYN.

In some embodiments, the anti-LAYN antibodies or antibody fragments thereof comprise a VH sequence that comprises an amino acid sequence with at least about 95%, preferably about 96%, more preferably about 97%, still more preferably about 98%, or even more preferably about 99%, sequence identity to the amino acid sequence set forth in SEQ ID NOs: 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188, preferably a VH sequence that comprises an amino acid sequence with at least about 95%, preferably about 96%, more preferably about 97%, still more preferably about 98%, or even more preferably about 99%, sequence identity to the amino acid sequence set forth in SEQ ID NOs: 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181. More preferably, the anti-LAYN antibodies or antibody fragments thereof retains the binding and/or functional activity of an anti-LAYN antibody or antibody fragment thereof that comprises the VH sequence of SEQ ID NOs: 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188. Even more preferably, the anti-LAYN antibodies or antibody fragments thereof retains the binding and/or functional activity of an anti-LAYN antibody or antibody fragment thereof that comprises the VH sequence of SEQ ID NOs: 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181. In some embodiments, the anti-LAYN antibodies or antibody fragments thereof comprise the VH sequence of SEQ ID NOs: 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188 and have one or more conservative amino acid substitutions, e.g., 1, 2, 3, 4, 5, 1-2, 1-3, 1-4 or 1-5 conservative amino acid substitutions in the VH sequence. In some embodiments, the anti-LAYN antibodies or antibody fragments thereof comprise the VH sequence of SEQ ID NOs: 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181 and have one or more conservative amino acid substitutions, e.g., 1, 2, 3, 4, 5, 1-2, 1-3, 1-4 or 1-5 conservative amino acid substitutions in the VH sequence. In some embodiments, the one or more conservative amino acid substitutions fall within one or more framework regions in SEQ ID NOs: 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188 (based on the numbering system of Kabat). In some embodiments, the one or more conservative amino acid substitutions fall within one or more framework regions in SEQ ID NOs: 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181 (based on the numbering system of Kabat).

In some embodiments, the anti-LAYN antibody or antibody fragment thereof comprises a VH sequence with at least about 95%, preferably about 96%, more preferably about 97%, still more preferably about 98%, or even more preferably about 99% sequence identity to the anti-LAYN VH sequence set forth in 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188 comprises one or more conservative amino acid substitutions in a framework region (based on the numbering system of Kabat), and retains the binding and/or functional activity of an anti-LAYN antibody or antibody fragment thereof that comprises a VH sequence as set forth in SEQ ID NOs: 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188 and a VL sequence as set forth in SEQ ID NOs: 8, 14, 25, 35, 39, 42, 45, 48, 51, 54, 56, 59, 62, 65, 68, 71, 73, 75, 79, 138, 170 or 179. In some embodiments, the anti-LAYN antibody or antibody fragment thereof comprises a VH sequence with at least about 95%, preferably about 96%, more preferably about 97%, still more preferably about 98%, or even more preferably about 99% sequence identity to the anti-LAYN VH sequence set forth in 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181 comprises one or more conservative amino acid substitutions in a framework region (based on the numbering system of Kabat), and retains the binding and/or functional activity of an anti-LAYN antibody or antibody fragment thereof that comprises a VH sequence as set forth in SEQ ID NOs: 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181 and a VL sequence as set forth in SEQ ID NOs: 8, 14, 25, 35, 39, 42, 45, 48, 51, 54, 56, 59, 62, 65, 68, 71, 73, 75, 79, 138, 170 or 179.

In some embodiments, the anti-LAYN antibodies or antibody fragments thereof comprise a VL sequence that comprises an amino acid sequence with at least about 95%, preferably about 96%, more preferably about 97%, still more preferably about 98%, or even more preferably about 99%, sequence identity to the amino acid sequence set forth in SEQ ID NOs: 8, 14, 25, 35, 39, 42, 45, 48, 51, 54, 56, 59, 62, 65, 68, 71, 73, 75, 79, 138, 170 or 179. Preferably, the anti-LAYN antibodies or antibody fragments thereof retains the binding and/or functional activity of an anti-LAYN antibody or antibody fragment thereof that comprises the VL sequence of SEQ ID NOs: 8, 14, 25, 35, 39, 42, 45, 48, 51, 54, 56, 59, 62, 65, 68, 71, 73, 75, 79, 138, 170 or 179. In some embodiments, the anti-LAYN antibodies or antibody fragments thereof comprise the VL sequence of SEQ ID NOs: 8, 14, 25, 35, 39, 42, 45, 48, 51, 54, 56, 59, 62, 65, 68, 71, 73, 75, 79, 138, 170 or 179 and have one or more conservative amino acid substitutions, e.g., 1, 2, 3, 4, 5, 1-2, 1-3, 1-4 or 1-5 conservative amino acid substitutions in the VL sequence. In some embodiments, the one or more conservative amino acid substitutions fall within one or more framework regions in SEQ ID NOs: 8, 14, 25, 35, 39, 42, 45, 48, 51, 54, 56, 59, 62, 65, 68, 71, 73, 75, 79, 138, 170 or 179 (based on the numbering system of Kabat).

In some embodiments, the anti-LAYN antibody or antibody fragment thereof comprises a VL sequence with at least about 95%, preferably about 96%, more preferably about 97%, still more preferably about 98%, or even more preferably about 99% sequence identity to the anti-LAYN VL sequence set forth in SEQ ID NOs: 8, 14, 25, 35, 39, 42, 45, 48, 51, 54, 56, 59, 62, 65, 68, 71, 73, 75, 79, 138, 170 or 179 comprises one or more conservative amino acid substitutions in a framework region (based on the numbering system of Kabat), and retains the binding and/or functional activity of an anti-LAYN antibody or antibody fragment thereof that comprises a VH sequence as set forth in SEQ ID NOs: 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188 and a VL sequence as set forth in SEQ ID NOs: 8, 14, 25, 35, 39, 42, 45, 48, 51, 54, 56, 59, 62, 65, 68, 71, 73, 75, 79, 138, 170 or 179.

In some embodiments, the anti-LAYN antibody, or the antigen-binding fragment thereof, as described herein, comprises a heavy chain variable region (VH) comprising the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188, wherein one or more, e.g., 1, 2, 3, 4, 5, 1-2, 1-3, 1-4 or 1-5, amino acids may be substituted; and a light chain variable region (VL) comprising the amino acid sequence of any one of SEQ ID NOs 8, 14, 25, 35, 39, 42, 45, 48, 51, 54, 56, 59, 62, 65, 68, 71, 73, 75, 79, 138, 170 or 179, wherein one or more, e.g., 1, 2, 3, 4, 5, 1-2, 1-3, 1-4 or 1-5, amino acids may be substituted.

Preferably, the anti-LAYN antibody, or the antigen-binding fragment thereof, as described herein, comprises
(i) a VH comprising a VH amino acid sequence of any one of antibodies A1 to A97 and P (e.g., as disclosed in Table 2), preferably of antibodies A1-A88, A90 and A91, wherein one or more, e.g., 1, 2, 3, 4, 5, 1-2, 1-3, 1-4 or 1-5, amino acids may be substituted; and
(ii) a VL comprising the corresponding VL amino acid sequence of the antibody selected in (i) (out of antibodies A1 to A97 and P (e.g., as disclosed in Table 2), preferably of antibodies A1-A88, A90 and A91), wherein one or more, e.g., 1, 2, 3, 4, 5, 1-2, 1-3, 1-4 or 1-5, amino acids may be substituted.

In some embodiments, the VH and/or the VL sequence of the anti-LAYN antibody, or the antigen-binding fragment thereof, corresponds to those of the exemplary antibodies as shown in Table 2 (i.e., without substitution of any amino acid).

In some embodiments, the anti-LAYN antibody, or the antigen-binding fragment thereof, as described herein, comprises a heavy chain variable region (VH) comprising the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188; and a light chain variable region (VL) comprising the amino acid sequence of any one of SEQ ID NOs 8, 14, 25, 35, 39, 42, 45, 48, 51, 54, 56, 59, 62, 65, 68, 71, 73, 75, 79, 138, 170 or 179.

Particularly preferably, the anti-LAYN antibody, or the antigen-binding fragment thereof, as described herein, comprises
(i) a VH comprising the VH amino acid sequence of any one of antibodies A1 to A97 and P (e.g., as disclosed in Table 2), preferably of antibodies A1-A88, A90 and A91; and
(ii) a VL comprising the corresponding VL amino acid sequence of the antibody selected in (i) (out of antibodies A1 to A97 and P (e.g., as disclosed in Table 2), preferably of antibodies A1-A88, A90 and A91).

Accordingly, it is preferred that the anti-LAYN antibody, or the antigen-binding fragment thereof, as described herein, comprises the specific combination of VH and VL sequences of any one of antibodies A1 to A97 and P (e.g., as disclosed in Table 2, preferably of antibodies A1-A88, A90 and A91).

More preferably, the anti-LAYN antibody, or an antigen-binding fragment thereof, comprises:
a heavy chain variable region comprising:
   a heavy chain complementary determining region 1 (HCDR1) having the amino acid sequence of SEQ ID NO: 1;
   a heavy chain complementary determining region 2 (HCDR2) having the amino acid sequence of NIX₂PX₃SGX₄TNYX₅X₆KFX₇X₈ (SEQ ID NO: 207), wherein
      X₂ is Y;
      X₃ is S or G;
      X₄ is T or S;
      X₅ is A;
      X₆ is Q;
      X₇ is Q; and
      X₈ is G; and
   a heavy chain complementary determining region 3 (HCDR3) having the amino acid sequence of X₉X₁₀YX₁₁RX₁₂AX₁₃X₁₄X₁₅ (SEQ ID NO: 208), wherein
      X₉ is E;
      X₁₀ is R;
      X₁₁ is G;
      X₁₂ is N;
      X₁₃ is A or;
      X₁₄ is E or D; and
      X₁₅ is Y; and
a light chain variable region comprising
   a light chain complementary determining region 1 (LCDR1) having the amino acid sequence of X₁₆ASQX₁₇₁X₁₈X₁₉X₂₀LA (SEQ ID NO: 209), wherein
      X₁₆ is R or L;
      X₁₇ is G or T;
      X₁₈ is G;
      X₁₉ is S or T; and
      X₂₀ is W;
   a light chain complementary determining region 2 (LCDR2) having the amino acid sequence of SEQ ID NO: 5; and
   a light chain complementary determining region 3 (LCDR3) having the amino acid sequence of SEQ ID NO: 6.

Based on specific preferred examples of such CDR sequences, the anti-LAYN antibody or the antigen-binding fragment preferably comprises:
a heavy chain variable region comprising an HCDR1 having the amino acid sequence of SEQ ID NO: 1, an HCDR2 having the amino acid sequence of any one of SEQ ID NOs 2, 11, or 19, and an HCDR3 having the amino acid sequence of any one of SEQ ID NOs 3, 32, or 142; and
a light chain variable region comprising an LCDR1 having the amino acid sequence of any one of SEQ ID NOs 4, 12, or 24, an LCDR2 having the amino acid sequence of SEQ ID NO: 5, and an LCDR3 having the amino acid sequence of SEQ ID NO: 6.

For example, the antibody or the antigen-binding fragment may comprise:
a heavy chain variable region comprising:
   an HCDR1 having the amino acid sequence of SEQ ID NO: 1, an HCDR2 having the amino acid sequence of SEQ ID NO: 2 and an HCDR3 having the amino acid sequence of SEQ ID NO: 3, or
   an HCDR1 having the amino acid sequence of SEQ ID NO: 1, an HCDR2 having the amino acid sequence of SEQ ID NO: 11 and an HCDR3 having the amino acid sequence of SEQ ID NO: 3, or
   an HCDR1 having the amino acid sequence of SEQ ID NO: 1, an HCDR2 having the amino acid sequence of SEQ ID NO: 19 and an HCDR3 having the amino acid sequence of SEQ ID NO: 142, or
   an HCDR1 having the amino acid sequence of SEQ ID NO: 1, an HCDR2 having the amino acid sequence of SEQ ID NO: 19 and an HCDR3 having the amino acid sequence of SEQ ID NO: 32; and
a light chain variable region comprising:
   an LCDR1 having the amino acid sequence of SEQ ID NO: 4, an LCDR2 having the amino acid sequence of SEQ ID NO: 5 and an LCDR3 having the amino acid sequence of SEQ ID NO: 6, or
   an LCDR1 having the amino acid sequence of SEQ ID NO: 12, an LCDR2 having the amino acid sequence of SEQ ID NO: 5 and an LCDR3 having the amino acid sequence of SEQ ID NO: 6, or
   an LCDR1 having the amino acid sequence of SEQ ID NO: 24, an LCDR2 having the amino acid sequence of SEQ ID NO: 5 and an LCDR3 having the amino acid sequence of SEQ ID NO: 6.

With regard to the VH and VL sequences, it is preferred that the anti-LAYN antibody, or the antigen-binding fragment thereof, comprises:
a heavy chain variable region (VH) comprising the amino acid sequence of QVQLX₂₆QX₂₇GX₂₈EX₂₉X₃₀X₃₁PGASVKX₃₂SCKASGYTFTX₁YWMDWVX₃₃QX₃₄PG X₃₅GLEWX₃₆GNIX₂PX₃SGX₄TNYX₅X₆KFX₇X₈X₃₇X₃₈TX₃₉TX₄₀DX₄₁X₄₂X₄₃X₄₄TX₄₅YM X₄₆LSX₄₇LX₄₈SX₄₉DX₅₀AX₅₁YYCX₅₂RX₉X₁₀YX₁₁RX₁₂AX₁₃X₁₄X₁₅WGQGTX₅₃VTVSS
(SEQ ID NO: 212), wherein
X₁ is S, X₂ is Y, X₃ is S or G, X₄ is T or S, X₅ is A, X₆ is Q, X₇ is Q, X₈ is G, X₉ is E, X₁₀ is R, X₁₁ is G, X₁₂ is N, X₁₃ is A or M, X₁₄ is E or D, X₁₅ is Y, X₂₆ is V, X₂₇ is S, X₂₈ is A or S, X₂₉ is V, X₃₀ is K, X₃₁ is K, X₃₂ is V, X₃₃ is R, X₃₄ is A, X₃₅ is Q, X₃₆ is M, X₃₇ is R, X₃₈ is V or A, X₃₉ is M or L, X₄₀ is V, X₄₁ is K, X₄₂ is S, X₄₃ is T, X₄₄ is S, X₄₅ is A, X₄₆ is E, X₄₇ is S, X₄₈ is R, X₄₉ is E, X₅₀ is T, X₅₁ is V, X₅₂ is A or T, and X₅₃ is L; and
a light chain variable region (VL) comprising the amino acid sequence of DIQMTQSPX₅₄SX₅₅SASX₅₆GX₅₇X₅₈VTITCX₁₆ASQX₁₇₁X₁₈X₁₉X₂₀LAWYQQKPGKX₅₉PX₆₀LLIYAAX₂₁SLX₂₂X₂₃GVPSRFSGSGSGTX₆₁FX₆₂X₆₃X₆₄ISSLQX₆₅EDFX₆₆X₆₇YY CQQX₂₄YSX₂₅PLTFGGGTKLEIK
(SEQ ID NO: 213), wherein
X₁₆ is R or L, X₁₇ is G or T, X₁₈ is G, X₁₉ is S or T, X₂₀ is W, X₂₁ is T, X₂₂ is A, X₂₃ is D, X₂₄ is L, X₂₅ is T, X₅₄ is S, X₅₅ is V, X₅₆ is V, X₅₇ is D, X₅₈ is R, X₅₉ is A or S, X₆₀ is K, X₆₁ is D, X₆₂ is T, X₆₃ is L or F, X₆₄ is T, X₆₅ is P, X₆₆ is A, and X₆₇ is T.

More preferably, the antibody or the antigen-binding fragment comprises a heavy chain variable region comprising an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to the amino acid sequence of any one of SEQ ID NOs 7, 13, 34 or 143 (the higher the %-identity, the more it is preferred); and a light chain variable region comprising an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical to the amino acid sequence of any one of SEQ ID NOs 8, 14 or 79 (the higher the %-identity, the more it is preferred).

Even more preferably, the anti-LAYN antibody or the antigen-binding fragment comprises a heavy chain variable region comprising an amino acid sequence of any one of SEQ ID NOs 7, 13, 34 or 143; and a light chain variable region comprising an amino acid sequence of any one of SEQ ID NOs 8, 14 or 79.

In some embodiments, the anti-LAYN antibody, or the antigen-binding fragment thereof, as described herein, comprises a heavy chain variable region comprising an HCDR1 having the amino acid sequence of SEQ ID NO: 1, an HCDR2 having the amino acid sequence of SEQ ID NO: 2, and an HCDR3 having the amino acid sequence of SEQ ID NO: 3; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6. Preferably, said anti-LAYN antibody, or the antigen-binding fragment thereof, comprises a heavy chain variable region comprising an amino acid sequence at least 80% identical to the amino acid sequence of SEQ ID NO: 7; and a light chain variable region comprising an amino acid sequence at least 80% identical to the amino acid sequence of SEQ ID NO: 8. More preferably, said anti-LAYN antibody, or the antigen-binding fragment thereof, comprises a heavy chain variable region comprising an amino acid sequence at least 85% identical to the amino acid sequence of SEQ ID NO: 7; and a light chain variable region comprising an amino acid sequence at least 85% identical to the amino acid sequence of SEQ ID NO: 8. Even more preferably, said anti-LAYN antibody, or the antigen-binding fragment thereof, comprises a heavy chain variable region comprising an amino acid sequence at least 90% identical to the amino acid sequence of SEQ ID NO: 7; and a light chain variable region comprising an amino acid sequence at least 90% identical to the amino acid sequence of SEQ ID NO: 8. Still more preferably, said anti-LAYN antibody, or the antigen-binding fragment thereof, comprises a heavy chain variable region comprising an amino acid sequence at least 95% identical to the amino acid sequence of SEQ ID NO: 7; and a light chain variable region comprising an amino acid sequence at least 95% identical to the amino acid sequence of SEQ ID NO: 8. Particularly preferably, said anti-LAYN antibody, or the antigen-binding fragment thereof, comprises a heavy chain variable region comprising the amino acid sequence of SEQ ID NO: 7; and a light chain variable region comprising the amino acid sequence of SEQ ID NO: 8. In embodiments, said anti-LAYN antibody, or an antigen-binding fragment thereof, binds to full length hLAYN isoform 2 (e.g., having the amino acid sequence of SEQ ID NO: 194) with a high binding affinity. In embodiments, high affinity as described herein includes wherein the K_{D} value is between about 0.001 nM and 50 nM, between about 0.01 nM and 50 nM, between about 0.1 nM and 50 nM, between about 1 nM and 50 nM, or less than about 50 nM. In embodiments, said anti-LAYN antibody, or an antigen-binding fragment thereof, has a high stability. In embodiments, high stability as described herein includes wherein the melting temperature or an inflection point (IP) of a thermal transition curve of the anti-LAYN antibody, or an antigen-binding fragment thereof, is between about 80 °C and 99 °C, between about 80 °C and 95 °C, between about 80 °C and 90 °C, or between about 80 °C and 85 °C, or greater than 80 °C.

In some embodiments, the anti-LAYN antibody, or the antigen-binding fragment thereof, as described herein, comprises a heavy chain variable region comprising an HCDR1 having the amino acid sequence of SEQ ID NO: 1, an HCDR2 having the amino acid sequence of SEQ ID NO: 11, and an HCDR3 having the amino acid sequence of SEQ ID NO: 3; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6. Preferably, said anti-LAYN antibody, or the antigen-binding fragment thereof, comprises a heavy chain variable region comprising an amino acid sequence at least 80% identical to the amino acid sequence of SEQ ID NO: 13; and a light chain variable region comprising an amino acid sequence at least 80% identical to the amino acid sequence of SEQ ID NO: 14. More preferably, said anti-LAYN antibody, or the antigen-binding fragment thereof, comprises a heavy chain variable region comprising an amino acid sequence at least 85% identical to the amino acid sequence of SEQ ID NO: 13; and a light chain variable region comprising an amino acid sequence at least 85% identical to the amino acid sequence of SEQ ID NO: 14. Even more preferably, said anti-LAYN antibody, or the antigen-binding fragment thereof, comprises a heavy chain variable region comprising an amino acid sequence at least 90% identical to the amino acid sequence of SEQ ID NO: 13; and a light chain variable region comprising an amino acid sequence at least 90% identical to the amino acid sequence of SEQ ID NO: 14. Still more preferably, said anti-LAYN antibody, or the antigen-binding fragment thereof, comprises a heavy chain variable region comprising an amino acid sequence at least 95% identical to the amino acid sequence of SEQ ID NO: 13; and a light chain variable region comprising an amino acid sequence at least 95% identical to the amino acid sequence of SEQ ID NO: 14. Particularly preferably, said anti-LAYN antibody, or the antigen-binding fragment thereof, comprises a heavy chain variable region comprising the amino acid sequence of SEQ ID NO: 13; and a light chain variable region comprising the amino acid sequence of SEQ ID NO: 14. In embodiments, said anti-LAYN antibody, or an antigen-binding fragment thereof, binds to full length hLAYN isoform 2 (e.g., having the amino acid sequence of SEQ ID NO: 194) with a high binding affinity. In embodiments, high affinity as described herein includes wherein the K_{D} value is between about 0.001 nM and 50 nM, between about 0.01 nM and 50 nM, between about 0.1 nM and 50 nM, between about 1 nM and 50 nM, or less than about 50 nM. In embodiments, said anti-LAYN antibody, or an antigen-binding fragment thereof, has a high stability. In embodiments, high stability as described herein includes wherein the melting temperature or an inflection point (IP) of a thermal transition curve of the anti-LAYN antibody, or an antigen-binding fragment thereof, is between about 80 °C and 99 °C, between about 80 °C and 95 °C, between about 80 °C and 90 °C, or between about 80 °C and 85 °C, or greater than 80 °C.

In some embodiments, the anti-LAYN antibody, or the antigen-binding fragment thereof, as described herein, comprises a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 142; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6. Preferably, said anti-LAYN antibody, or the antigen-binding fragment thereof, comprises a heavy chain variable region comprising an amino acid sequence at least 80% identical to the amino acid sequence of SEQ ID NO: 143; and a light chain variable region comprising an amino acid sequence at least 80% identical to the amino acid sequence of SEQ ID NO: 79. More preferably, said anti-LAYN antibody, or the antigen-binding fragment thereof, comprises a heavy chain variable region comprising an amino acid sequence at least 85% identical to the amino acid sequence of SEQ ID NO: 143; and a light chain variable region comprising an amino acid sequence at least 85% identical to the amino acid sequence of SEQ ID NO: 79. Even more preferably, said anti-LAYN antibody, or the antigen-binding fragment thereof, comprises a heavy chain variable region comprising an amino acid sequence at least 90% identical to the amino acid sequence of SEQ ID NO: 143; and a light chain variable region comprising an amino acid sequence at least 90% identical to the amino acid sequence of SEQ ID NO: 79. Still more preferably, said anti-LAYN antibody, or the antigen-binding fragment thereof, comprises a heavy chain variable region comprising an amino acid sequence at least 95% identical to the amino acid sequence of SEQ ID NO: 143; and a light chain variable region comprising an amino acid sequence at least 95% identical to the amino acid sequence of SEQ ID NO: 79. Particularly preferably, said anti-LAYN antibody, or the antigen-binding fragment thereof, comprises a heavy chain variable region comprising the amino acid sequence of SEQ ID NO: 143; and a light chain variable region comprising the amino acid sequence of SEQ ID NO: 79. In embodiments, said anti-LAYN antibody, or an antigen-binding fragment thereof, binds to full length hLAYN isoform 2 (e.g., having the amino acid sequence of SEQ ID NO: 194) with a high binding affinity. In embodiments, high affinity as described herein includes wherein the K_{D} value is between about 0.001 nM and 50 nM, between about 0.01 nM and 50 nM, between about 0.1 nM and 50 nM, between about 1 nM and 50 nM, or less than about 50 nM. In embodiments, said anti-LAYN antibody, or an antigen-binding fragment thereof, has a high stability. In embodiments, high stability as described herein includes wherein the melting temperature or an inflection point (IP) of a thermal transition curve of the anti-LAYN antibody, or an antigen-binding fragment thereof, is between about 80 °C and 99 °C, between about 80 °C and 95 °C, between about 80 °C and 90 °C, or between about 80 °C and 85 °C, or greater than 80 °C.

In some embodiments, the anti-LAYN antibody, or the antigen-binding fragment thereof, as described herein, comprises a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6. Preferably, said anti-LAYN antibody, or the antigen-binding fragment thereof, comprises a heavy chain variable region comprising an amino acid sequence at least 80% identical to the amino acid sequence of SEQ ID NO: 34; and a light chain variable region comprising an amino acid sequence at least 80% identical to the amino acid sequence of SEQ ID NO: 79. More preferably, said anti-LAYN antibody, or the antigen-binding fragment thereof, comprises a heavy chain variable region comprising an amino acid sequence at least 85% identical to the amino acid sequence of SEQ ID NO: 34; and a light chain variable region comprising an amino acid sequence at least 85% identical to the amino acid sequence of SEQ ID NO: 79. Even more preferably, said anti-LAYN antibody, or the antigen-binding fragment thereof, comprises a heavy chain variable region comprising an amino acid sequence at least 90% identical to the amino acid sequence of SEQ ID NO: 34; and a light chain variable region comprising an amino acid sequence at least 90% identical to the amino acid sequence of SEQ ID NO: 79. Still more preferably, said anti-LAYN antibody, or the antigen-binding fragment thereof, comprises a heavy chain variable region comprising an amino acid sequence at least 95% identical to the amino acid sequence of SEQ ID NO: 34; and a light chain variable region comprising an amino acid sequence at least 95% identical to the amino acid sequence of SEQ ID NO: 79. Particularly preferably, said anti-LAYN antibody, or the antigen-binding fragment thereof, comprises a heavy chain variable region comprising the amino acid sequence of SEQ ID NO: 34; and a light chain variable region comprising the amino acid sequence of SEQ ID NO: 79. In embodiments, said anti-LAYN antibody, or an antigen-binding fragment thereof, binds to full length hLAYN isoform 2 (e.g., having the amino acid sequence of SEQ ID NO: 194) with a high binding affinity. In embodiments, high affinity as described herein includes wherein the K_{D} value is between about 0.001 nM and 50 nM, between about 0.01 nM and 50 nM, between about 0.1 nM and 50 nM, between about 1 nM and 50 nM, or less than about 50 nM. In embodiments, said anti-LAYN antibody, or an antigen-binding fragment thereof, has a high stability. In embodiments, high stability as described herein includes wherein the melting temperature or an inflection point (IP) of a thermal transition curve of the anti-LAYN antibody, or an antigen-binding fragment thereof, is between about 80 °C and 99 °C, between about 80 °C and 95 °C, between about 80 °C and 90 °C, or between about 80 °C and 85 °C, or greater than 80 °C.

### Constant Regions, Antibody Fragments and Full-Length Antibodies

In some embodiments, the anti-LAYN antibody, or the antigen-binding fragment thereof, as described herein, comprises a heavy chain constant region selected from IgG₁, IgG₂, IgG₃, IgG₄, IgM, IgA and IgE or a variant thereof and/or a light chain constant region selected from the group consisting of a kappa and a lambda light chain.

The constant domains or constant regions are not directly involved in antigen binding. Instead, the constant regions typically mediate various Ig effector functions. Non-limiting examples thereof include participation of the antibody in antibody-dependent cellular cytotoxicity (ADCC), complement-dependent cytotoxicity (CDC) and antibody-dependent cellular phagocytosis (ADCP).

The light chains of vertebrate immunoglobulins are assigned to one of two clearly distinct classes, kappa (κ) and lambda (λ), based on the amino acid sequence of the constant domain. By comparison, the heavy chains of mammalian immunoglobulins are assigned to one of five major classes, according to the sequence of the constant domains: IgA, IgD, IgE, IgG, and IgM. The heavy chain constant domains that correspond to the different classes of immunoglobulins are called α, δ, ε, γ, and μ, respectively. The subunit structures and three-dimensional configurations of the classes of native immunoglobulins are well known.

IgG and IgA are further divided into subclasses (isotypes), e.g., IgG₁, IgG₂, IgG₃, IgG₄, IgA₁, and IgA₂, respectively. The antibodies, as described herein, may be of any one these classes or subclasses. Preferably, the antibody is of the IgG-type, more preferably IgG1. Accordingly, the antibody preferably comprises antibody comprises an IgG1 heavy chain constant region. While human IgG, in particular human IgG1 (also referred to herein as “huIgG1” or “hIgG1”), is preferred, the constant region may be of any allotype.

In some embodiments, the antibody comprises at least a portion of an immunoglobulin Fc region, preferably that of a human immunoglobulin. Ordinarily, the antibody will contain both the light chain as well as at least the variable domain of a heavy chain. The antibody also may include one or more of the C_{H}1, hinge, C_{H}2, C_{H}3, and/or C_{H}4 regions of the heavy chain, as appropriate.

Preferably, the antibody includes a (full) Fc region. Specifically, the heavy chain of the antibody may include C_{H}1, hinge, C_{H}2 and C_{H}3 region and the light chain of the antibody may include a light chain constant region (C_{L}). An exemplary sequence for human IgG1 CH1-CH2-CH3 is provided in SEQ ID NO: 173 and an exemplary sequence for human CL is provided in SEQ ID NO: 174. Preferably, the anti-LAYN antibody or antigen binding fragment thereof according to any one of the previous claims, wherein said antibody comprises a heavy chain comprising an amino acid sequence at least 95% identical to the amino acid sequence of SEQ ID NO: 173; and a light chain comprising an amino acid sequence at least 95% identical to the amino acid sequence of SEQ ID NO: 174.

In some embodiments, the anti-LAYN antibody of the present disclosure comprises a human wild-type IgG Fc region, preferably a human wild-type IgG1 Fc region. More preferably, the heavy chain (HC) of the anti-LAYN antibody of the present disclosure comprises SEQ ID NO: 173 and/or the light chain (LC) of the anti-LAYN antibody of the present disclosure comprises SEQ ID NO: 174. These sequences are comprised in the HC and LC sequences of exemplary antibodies A1-A104, as depicted in Table 2.

As described above, the present disclosure also includes variant antibodies or antigen-binding fragments thereof. Accordingly, the anti-LAYN antibody, or the antigen-binding fragment thereof, as described herein, may comprise a heavy chain comprising an amino acid sequence at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146, 182, 185, 187 or 189; and a light chain comprising an amino acid sequence at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 10, 16, 26, 37, 40, 43, 46, 49, 52, 55, 57, 60, 63, 66, 69, 72, 74, 76, 81, 139 or 180. Preferably, the anti-LAYN antibody, or the antigen-binding fragment thereof, as described herein, may comprise a heavy chain comprising an amino acid sequence at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146 or 182; and a light chain comprising an amino acid sequence at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 10, 16, 26, 37, 40, 43, 46, 49, 52, 55, 57, 60, 63, 66, 69, 72, 74, 76, 81, 139 or 180. In embodiments, said anti-LAYN antibody, or an antigen-binding fragment thereof, binds to full length hLAYN isoform 2 (e.g., having the amino acid sequence of SEQ ID NO: 194) with a high binding affinity. In embodiments, high affinity as described herein includes wherein the K_{D} value is between about 0.001 nM and 50 nM, between about 0.01 nM and 50 nM, between about 0.1 nM and 50 nM, between about 1 nM and 50 nM, or less than about 50 nM. In embodiments, said anti-LAYN antibody, or an antigen-binding fragment thereof, has a high stability. In embodiments, high stability as described herein includes wherein the melting temperature or an inflection point (IP) of a thermal transition curve of the anti-LAYN antibody, or an antigen-binding fragment thereof, is between about 80 °C and 99 °C, between about 80 °C and 95 °C, between about 80 °C and 90 °C, or between about 80 °C and 85 °C, or greater than 80 °C.

As noted above, it is to be understood that in the variant anti-LAYN antibodies and antibody fragments the amino acid sequence of the CDRs remain unchanged or have minimal changes (e.g., no more than 1, 2, 3, 4 or 5 changes). Preferably, the variant anti-LAYN antibodies and antigen-binding fragments include the specific sets of six CDRs of antibodies A1 to A97 and P, e.g., as described in Tables 2-8 (without any changes to the CDR sequences), preferably of antibodies A1-A88, A90 and A91, even more preferably of antibodies A1, A2, A85 and A86. In some embodiments, the sequences of the six CDRs and of the constant regions of any one of exemplary antibodies P and A1 to A97 (preferably of antibodies A1-A88, A90 and A91, more preferably of antibodies A1, A2, A85 and A86) are maintained, but one or more changes (mutations) are introduced into one or more of the (VH and/or VL) framework region(s). In some embodiments, the sequences of the variable regions (VH and VL) of any one of exemplary antibodies P and A1 to A97 (preferably of antibodies A1-A88, A90 and A91, more preferably of antibodies A1, A2, A85 and A86) are maintained, but one or more changes (mutations) are introduced into one or more of the constant region(s). In some embodiments, the CDRs of any one of exemplary antibodies P and A1 to A97 (preferably of antibodies A1-A88, A90 and A91, more preferably of antibodies A1, A2, A85 and A86) are maintained, but two or more changes (mutations) are introduced (i) into one or more of the (VH and/or VL) framework region(s); and (ii) into one or more of the constant region(s).

Preferably, the anti-LAYN antibody, or the antigen-binding fragment thereof, as described herein, comprises
(i) a heavy chain (HC) comprising an amino acid sequence at least 80% identical to a HC amino acid sequence of any one of antibodies A1 to A97 and P (e.g., as disclosed in Table 2), preferably of antibodies A1-A88, A90 and A91, more preferably of antibodies A1, A2, A85 and A86; and
(ii) a light chain (LC) comprising an amino acid sequence at least 80% identical to the corresponding LC amino acid sequence of the antibody selected in (i) (out of antibodies A1 to A97 and P (e.g., as disclosed in Table 2), preferably of antibodies A1-A88, A90 and A91, more preferably of antibodies A1, A2, A85 and A86).

In some embodiments, the anti-LAYN antibody, or the antigen-binding fragment thereof, as described herein, may comprise a heavy chain comprising an amino acid sequence at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146, 182, 185, 187 or 189; and a light chain comprising an amino acid sequence at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 10, 16, 26, 37, 40, 43, 46, 49, 52, 55, 57, 60, 63, 66, 69, 72, 74, 76, 81, 139 or 180. Preferably, the anti-LAYN antibody, or the antigen-binding fragment thereof, as described herein, may comprise a heavy chain comprising an amino acid sequence at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146 or 182; and a light chain comprising an amino acid sequence at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99% or 100%) identical to the amino acid sequence of any one of SEQ ID NOS 10, 16, 26, 37, 40, 43, 46, 49, 52, 55, 57, 60, 63, 66, 69, 72, 74, 76, 81, 139 or 180. In embodiments, said anti-LAYN antibody, or an antigen-binding fragment thereof, binds to full length hLAYN isoform 2 (e.g., having the amino acid sequence of SEQ ID NO: 194) with a high binding affinity. In embodiments, high affinity as described herein includes wherein the K_{D} value is between about 0.001 nM and 50 nM, between about 0.01 nM and 50 nM, between about 0.1 nM and 50 nM, between about 1 nM and 50 nM, or less than about 50 nM. In embodiments, said anti-LAYN antibody, or an antigen-binding fragment thereof, has a high stability. In embodiments, high stability as described herein includes wherein the melting temperature or an inflection point (IP) of a thermal transition curve of the anti-LAYN antibody, or an antigen-binding fragment thereof, is between about 80 °C and 99 °C, between about 80 °C and 95 °C, between about 80 °C and 90 °C, or between about 80 °C and 85 °C, or greater than 80 °C.

More preferably, the anti-LAYN antibody, or the antigen-binding fragment thereof, as described herein, comprises
(i) an HC comprising an amino acid sequence at least 85% identical to a HC amino acid sequence of any one of antibodies A1 to A97 and P (e.g., as disclosed in Table 2), preferably of antibodies A1-A88, A90 and A91, more preferably of antibodies A1, A2, A85 and A86; and
(ii) an LC comprising an amino acid sequence at least 85% identical to the corresponding LC amino acid sequence of the antibody selected in (i) (out of antibodies A1 to A97 and P (e.g., as disclosed in Table 2), preferably of antibodies A1-A88, A90 and A91, more preferably of antibodies A1, A2, A85 and A86).

In some embodiments, the anti-LAYN antibody, or the antigen-binding fragment thereof, as described herein, may comprise a heavy chain comprising an amino acid sequence at least 90% (e.g., at least 91%, at least 92%, at least 93%, at least 94%, at least 95%, at least 96%, at least 97%, at least 98%, at least 99% or 100%) identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146, 182, 185, 187 or 189; and a light chain comprising an amino acid sequence at least 90% (e.g., at least 91%, at least 92%, at least 93%, at least 94%, at least 95%, at least 96%, at least 97%, at least 98%, at least 99% or 100%) identical to the amino acid sequence of any one of SEQ ID NOs 10, 16, 26, 37, 40, 43, 46, 49, 52, 55, 57, 60, 63, 66, 69, 72, 74, 76, 81, 139 or 180. Preferably, the anti-LAYN antibody, or the antigen-binding fragment thereof, as described herein, may comprise a heavy chain comprising an amino acid sequence at least 90% (e.g., at least 91%, at least 92%, at least 93%, at least 94%, at least 95%, at least 96%, at least 97%, at least 98%, at least 99% or 100%) identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146 or 182; and a light chain comprising an amino acid sequence at least 90% (e.g., at least 91%, at least 92%, at least 93%, at least 94%, at least 95%, at least 96%, at least 97%, at least 98%, at least 99% or 100%) identical to the amino acid sequence of any one of SEQ ID NOs 10, 16, 26, 37, 40, 43, 46, 49, 52, 55, 57, 60, 63, 66, 69, 72, 74, 76, 81, 139 or 180. In embodiments, said anti-LAYN antibody, or an antigen-binding fragment thereof, binds to full length hLAYN isoform 2 (e.g., having the amino acid sequence of SEQ ID NO: 194) with a high binding affinity. In embodiments, high affinity as described herein includes wherein the K_{D} value is between about 0.001 nM and 50 nM, between about 0.01 nM and 50 nM, between about 0.1 nM and 50 nM, between about 1 nM and 50 nM, or less than about 50 nM. In embodiments, said anti-LAYN antibody, or an antigen-binding fragment thereof, has a high stability. In embodiments, high stability as described herein includes wherein the melting temperature or an inflection point (IP) of a thermal transition curve of the anti-LAYN antibody, or an antigen-binding fragment thereof, is between about 80 °C and 99 °C, between about 80 °C and 95 °C, between about 80 °C and 90 °C, or between about 80 °C and 85 °C, or greater than 80 °C.

Even more preferably, the anti-LAYN antibody, or the antigen-binding fragment thereof, as described herein, comprises
(i) an HC comprising an amino acid sequence at least 90% identical to a HC amino acid sequence of any one of antibodies A1 to A97 and P (e.g., as disclosed in Table 2), preferably of antibodies A1-A88, A90 and A91, more preferably of antibodies A1, A2, A85 and A86; and
(ii) an LC comprising an amino acid sequence at least 90% identical to the corresponding LC amino acid sequence of the antibody selected in (i) (out of antibodies A1 to A97 and P (e.g., as disclosed in Table 2), preferably of antibodies A1-A88, A90 and A91, more preferably of antibodies A1, A2, A85 and A86).

In some embodiments, the anti-LAYN antibody, or the antigen-binding fragment thereof, as described herein, may comprise a heavy chain comprising an amino acid sequence at least 95% (e.g., at least 96%, at least 97%, at least 98%, at least 99% or 100%) identical to the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146, 182, 185, 187 or 189; and a light chain comprising an amino acid sequence at least 95% (e.g., at least 96%, at least 97%, at least 98%, at least 99% or 100%) identical to the amino acid sequence of any one of SEQ ID NOs 10, 16, 26, 37, 40, 43, 46, 49, 52, 55, 57, 60, 63, 66, 69, 72, 74, 76, 81, 139 or 180. In embodiments, said anti-LAYN antibody, or an antigen-binding fragment thereof, binds to full length hLAYN isoform 2 (e.g., having the amino acid sequence of SEQ ID NO: 194) with a high binding affinity. In embodiments, high affinity as described herein includes wherein the K_{D} value is between about 0.001 nM and 50 nM, between about 0.01 nM and 50 nM, between about 0.1 nM and 50 nM, between about 1 nM and 50 nM, or less than about 50 nM. In embodiments, said anti-LAYN antibody, or an antigen-binding fragment thereof, has a high stability. In embodiments, high stability as described herein includes wherein the melting temperature or an inflection point (IP) of a thermal transition curve of the anti-LAYN antibody, or an antigen-binding fragment thereof, is between about 80 °C and 99 °C, between about 80 °C and 95 °C, between about 80 °C and 90 °C, or between about 80 °C and 85 °C, or greater than 80 °C.

Still more preferably, the anti-LAYN antibody, or the antigen-binding fragment thereof, as described herein, comprises
(i) an HC comprising an amino acid sequence at least 95% identical to a HC amino acid sequence of any one of antibodies A1 to A97 and P (e.g., as disclosed in Table 2), preferably of antibodies A1-A88, A90 and A91, more preferably of antibodies A1, A2, A85 and A86; and
(ii) an LC comprising an amino acid sequence at least 95% identical to the corresponding LC amino acid sequence of the antibody selected in (i) (out of antibodies A1 to A97 and P (e.g., as disclosed in Table 2), preferably of antibodies A1-A88, A90 and A91, more preferably of antibodies A1, A2, A85 and A86).

In some embodiments, the anti-LAYN antibody, or the antigen-binding fragment thereof, as described herein, comprises a heavy chain (HC) comprising the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146, 182, 185, 187 or 189; and a light chain (LC) comprising the amino acid sequence of any one of SEQ ID NOs 10, 16, 26, 37, 40, 43, 46, 49, 52, 55, 57, 60, 63, 66, 69, 72, 74, 76, 81, 139 or 180.

Particularly preferably, the anti-LAYN antibody, or the antigen-binding fragment thereof, as described herein, comprises
(i) an HC comprising the HC amino acid sequence of any one of antibodies A1 to A97 and P (e.g., as disclosed in Table 2), preferably of antibodies A1-A88, A90 and A91, more preferably of antibodies A1, A2, A85 and A86; and
(ii) an LC comprising the corresponding LC amino acid sequence of the antibody selected in (i) (out of antibodies A1 to A97 and P (e.g., as disclosed in Table 2), preferably of antibodies A1-A88, A90 and A91, more preferably of antibodies A1, A2, A85 and A86).

Accordingly, it is preferred that the anti-LAYN antibody, or the antigen-binding fragment thereof, as described herein, comprises the specific combination of HC and LC sequences of any one of antibodies A1 to A97 and P (e.g., as disclosed in Table 2), preferably of antibodies A1-A88, A90 and A91, more preferably of antibodies A1, A2, A85 and A86.

Particularly preferably, the anti-LAYN antibody, or the antigen-binding fragment thereof, as described herein, comprises a heavy chain comprising the amino acid sequence of SEQ ID NO: 9; and a light chain comprising the amino acid sequence of SEQ ID NO: 10.

Particularly preferably, the anti-LAYN antibody, or the antigen-binding fragment thereof, as described herein, comprises a heavy chain comprising the amino acid sequence of SEQ ID NO: 15; and a light chain comprising the amino acid sequence of SEQ ID NO: 16.

Particularly preferably, the anti-LAYN antibody, or the antigen-binding fragment thereof, as described herein, comprises a heavy chain comprising the amino acid sequence of SEQ ID NO: 144; and a light chain comprising the amino acid sequence of SEQ ID NO: 81.

Particularly preferably, the anti-LAYN antibody, or the antigen-binding fragment thereof, as described herein, comprises a heavy chain comprising the amino acid sequence of SEQ ID NO: 36; and a light chain comprising the amino acid sequence of SEQ ID NO: 81.

As described above, the Fc region of an antibody mediates its serum half-life and effector functions, such as complement-dependent cytotoxicity (CDC), antibody-dependent cellular cytotoxicity (ADCC) and antibody-dependent cell phagocytosis (ADCP). To optimize such antibody properties mediated by the Fc region, Fc engineering can be employed. In the context of the present disclosure, antibodies having increased effector function, such as increased ADCC and/or ADCP, are preferred. Thereby, increased reduction or depletion of (intratumoral) regulatory T cells expressing LAYN may be achieved.

In some embodiments, the constant region or Fc region may include one or more, e.g., 1, 2, 3, 4, 5, 6, 7, 8, 9, 10 or more, mutations as compared to a wild-type Fc region (e.g., compared to human wild-type IgG1, e.g. of SEQ ID NO: 173 and/or 174). Various mutations are known in the art for constant regions/Fc regions of antibodies, e.g., to increase or reduce serum half-life and/or effector function. Usually, mutations in the Fc region of an antibody are indicated according to the EU-index of Kabat. As used herein, “EU index of Kabat” (also referred to as “EU index as in Kabat”, “EU numbering system of Kabat” or the like) will be understood to mean the numbering of an antibody heavy chain is according to the EU index as taught in Kabat et al., Sequences of Proteins of Immunological Interest, Vol. 1, 5th ed., 1991, Bethesda, MD: U.S. Dept. of Health and Human Services, Public Health Service, National Institutes of Health, NIH Publication No. 91-3242. The EU index is based on the residue numbering of the human IgG1 EU antibody.

Preferably, the anti-LAYN antibody or the antigen-binding fragment thereof comprises an engineered Fc region, which mediates increased effector function (in comparison to the respective wild-type Fc region), such as increased ADCC and/or ADCP. Various Fc mutations are known in the art to increase effector function, such as ADCC and/or ADCP. Preferably, the anti-LAYN antibody or the antigen-binding fragment thereof is afucosylated (to increase effector function).

In some embodiments, the anti-LAYN antibody or antigen-binding fragment thereof comprises a heavy chain constant region comprising G236A/A330L/1332E (also referred to herein under the name of “GAALIE”) substitutions (according to the EU-index of Kabat). These set of three mutations (G236A, A330L and 1332E) are known in the art to increase effector functions, in particular ADCC. For example, the anti-LAYN antibody or antigen-binding fragment thereof comprises a human IgG1 heavy chain constant region comprising the G236A/A330L/1332E substitutions (according to the EU-index of Kabat). More specifically, the heavy chain constant regions of the anti-LAYN antibody may differ from SEQ ID NO 174 (only) in that it comprises the substitutions G236A/A330L/1332E. A heavy chain of an exemplified anti-LAYN antibody comprises SEQ ID NO: 171.

In some embodiments, the anti-LAYN antibody or antigen-binding fragment thereof comprises a heavy chain constant region comprising S239D/1332E (also referred to herein under the name of “SDIE”) substitutions (according to the EU-index of Kabat). These set of two mutations (S239D and 1332E) are also known in the art to increase effector functions, in particular ADCC. For example, the anti-LAYN antibody or antigen-binding fragment thereof comprises a human IgG1 heavy chain constant region comprising the S239D/1332E substitutions (according to the EU-index of Kabat). More specifically, the heavy chain constant regions of the anti-LAYN antibody may differ from SEQ ID NO 174 (only) in that it comprises the substitutions S239D/1332E. A heavy chain of an exemplified anti-LAYN antibody comprises SEQ ID NO: 172.

In some aspects, the anti-LAYN antibodies and antigen-binding fragments thereof can include modifications, such as glycosylation, oxidation, or deamidation.

Accordingly, another type of amino acid variant of the antibody involves altering the original glycosylation pattern of the antibody. The term “altering” in this context means deleting one or more carbohydrate moieties found in the antibody, and/or adding one or more glycosylation sites that were not previously present in the antibody. For example, an antibody may comprise an amino acid substitution at position 297 of the human IgG₁ heavy chain to abrogate oligosaccharyltransferase enzyme complex-mediated glycosylation by replacing the asparagine 297 (e.g. N297A, N297G).

Removal of any carbohydrate moieties present on the antibody can be accomplished, for example, chemically or enzymatically. Chemical deglycosylation is described by Hakimuddin et al., 1987, Arch. Biochem. Biophys. 259:52 and by Edge et al., 1981, Anal. Biochem., 118:131. Enzymatic cleavage of carbohydrate moieties on antibodies can be achieved by the use of a variety of endo- and exo-glycosidases as described by Thotakura et al., 1987, Meth. Enzymol 138:350. Other options include engineering of cells producing the antibodies, e.g. by deleting (“knock-out” of) enzymes of those cells, which are involved in establishing carbohydrate moieties on the antibody.

Preferably, the anti-LAYN antibody, or the antigen-binding fragment thereof, is afucosylated. As used herein “afucosylation” refers to reducing or abolishing fucosylation of a molecule, e.g., an antibody. In some embodiments, “afucosylated” antibodies are (a plurality of) antibodies, wherein in at least 50%, preferably at least 60%, more preferably at least 70%, even more preferably at least 80%, still more preferably at least 90% and particularly preferably at least 95% of the antibody molecules (having a reduced amount of fucose, e.g. on its glycomoieties, or) lacking fucose. Afucosylation is known in the art to increase ADCC. More specifically, removal of the core fucose from the biantennary complex-type oligosaccharides attached to the Fc is known to increased ADCC effector function without altering antigen binding or CDC effector function. Various methods are known in the art to obtain afucosylated antibodies. These include expression in non-mammlian cells, such as prokaryotic cells (e.g., E. coli), yeast or plant cells. Preferably, mammalian cell lines engineered for expression of afucosylated antibodies are used, such as a FUT8 knockout cell line, a cell line comprising a small interfering RNA specifically against the FUT8 gene and a CHO cell line expressing GDP-6-deoxy-D-lyxo-4-hexylose reductase (RMD) (RMD-CHO). In some embodiments, an afucosylated antibody may be obtained as described in WO 2011/035884 A1, which is incorporated herein by reference. Preferably, afucoslyated anti-LAYN antibodies are obtained by expression in a cell, which lacks fucosyltransferase 8 (Fut8). Fut8 is the enzyme responsible for transferring fucose on the glycosylation tree in the Golgi apparatus. Specifically, afucoslyated anti-LAYN antibodies may be obtained by expression in a Fut8/(knockout) CHO cell line. This is described, for example, in Malphettes et al., 2010 (Malphettes L, Freyvert Y, Chang J, Liu P Q, Chan E, Miller J C, Zhou Z, Nguyen T, Tsai C, Snowden A W, Collingwood T N, Gregory P D, Cost G J. Highly efficient deletion of FUT8 in CHO cell lines using zinc-finger nucleases yields cells that produce completely nonfucosylated antibodies. Biotechnol Bioeng. 2010 Aug. 1; 106(5):774-83. doi: 10.1002/bit.22751), which is incorporated herein by reference.

In some embodiments, the anti-LAYN antibody or the antigen binding fragment comprises a heavy chain lacking a C-terminal lysine residue or a heavy chain including a C-terminal lysine residue. The C-terminal lysine residue of the heavy chain may also be referred to as Lys447 (according to the EU-index of Kabat), in particular with regard to IgG1. In some embodiments, the nucleic acid encoding the antibody heavy chain does not encode the C-terminal lysine. Accordingly, the antibody may then be expressed without the C-terminal lysine (of the wild-type IgG1 sequence). In other embodiments, the nucleic acid encoding the antibody may include the C-terminal lysine and the antibody may then be expressed including the C-terminal lysine, which thereafter may be proteolytically cleaved upon the expression of the antibody. While both scenarios result in antibodies lacking the C-terminal lysine (e.g., Lys447 of wild-type IgG1), the latter scenario (proteolytic cleavage) may result in a mixture of antibodies with and without C-terminal lysine, depending on the effectiveness of the proteolytic cleavage. In other embodiments, the anti-LAYN antibody may include the C-terminal lysine. For example, the amino acid sequence according to SEQ ID NO: 174 or the heavy chain (HC) sequences of antibodies P and A1-A104 depicted in Table 2 may additionally include a C-terminal lysine (which may be directly linked to the respective sequences, e.g., by a peptide bond).

A type of useful modification comprises linking the antibody to one of a variety of nonproteinaceous polymers, e.g., polyethylene glycol, polypropylene glycol, or polyoxyalkylenes, in the manner set forth in one or more of U.S. Pat. Nos. 4,640,835, 4,496,689, 4,301,144, 4,670,417, 4,791,192 and 4,179,337.

In some embodiments, the anti-LAYN antibody, or the antigen binding fragment thereof, is a monoclonal antibody.

In some embodiments, the anti-LAYN antibody, or the antigen binding fragment thereof, is a monospecific antibody.

In some embodiments, the anti-LAYN antibody, or the antigen binding fragment thereof, is a human, humanized, or chimeric antibody, preferably a humanized antibody.

In certain embodiments, the anti-LAYN antibody is an antibody fragment. There are techniques that have been developed for the production of antibody fragments. Fragments can be derived via proteolytic digestion of intact antibodies (see, e.g., Morimoto et al., 1992, Journal of Biochemical and Biophysical Methods 24:107-117; and Brennan et al., 1985, Science 229:81). Alternatively, the fragments can be produced directly in recombinant host cells. For example, Fab′-SH fragments can be directly recovered from E. coli and chemically coupled to form F(ab′)₂ fragments (see, e.g., Carter et al., 1992, Bio/Technology 10:163-167). By another approach, F(ab′)₂ fragments can be isolated directly from recombinant host cell culture. Other techniques for the production of antibody fragments will be apparent to a skilled practitioner.

In certain embodiments, it may be desirable to use an anti-LAYN antibody fragment, rather than an intact antibody. It may be desirable to modify the antibody fragment in order to increase its serum half-life. This can be achieved, for example, by incorporation of a salvage receptor binding epitope into the antibody fragment. In one method, the appropriate region of the antibody fragment can be altered (e.g., mutated), or the epitope can be incorporated into a peptide tag that is then fused to the antibody fragment at either end or in the middle, for example, by DNA or peptide synthesis (see, e.g., WO 96/32478). For example, antibody fragments of the invention may also be fused to human serum albumin to increase the serum half-life, if the use of a full length IgG scaffold is undesirable. Such fusion proteins of the antibody fragment with human serum albumin may be advantageous in situations in which two different antibody fragments need to be fused to increase avidity, or to generate a bispecific binding protein with extended serum half-life (see e.g. WO 05/077042 A2).

In some embodiments, the anti-LAYN antibody fragment may be selected from a Fab, Fab′, F(ab′)₂, Fd, Fv, scFv and scFv-Fc fragment.

In some embodiments, the anti-LAYN antibody or antigen-binding fragment thereof binds the extracellular domain of human LAYN isoform 2 (such as SEQ ID NO: 190) at pH 7.4 with a binding affinity (K_{D}) of 50 nM or lower, 40 nM or lower, 30 nM or lower, 25 nM or lower, 20 nM or lower, 15 nM or lower, or 10 nM or lower.

In some embodiments, the anti-LAYN antibody or antigen-binding fragment thereof binds the extracellular domain of human LAYN isoform 2 (such as SEQ ID NO: 190) at pH 6.0 with a binding affinity (K_{D}) of 50 nM or lower, 40 nM or lower, 30 nM or lower, 25 nM or lower, 20 nM or lower.

Binding affinity may be determined by surface plasmon resonance analysis (SPR; Malmqvist M., “Surface plasmon resonance for detection and measurement of antibody-antigen affinity and kinetics.”, Curr Opin Immunol. 1993 April; 5(2):282-6.). A specific exemplified method for performing SPR is described in Example 3.

The present invention also provides the anti-LAYN antibody or antigen-binding fragment thereof that competes for binding to LAYN with an anti-LAYN antibody according to the present invention, especially with any one of the exemplary antibodies P and A1-A97, preferably with any one of antibodies A1-A88, A90 and A91, more preferably with any one of antibodies A1, A2, A85 and A86. In some embodiments, the present disclosure provides an anti-LAYN antibody, or an antigen-binding fragment thereof, that specifically binds to human LAYN isoform 2, wherein the antibody or antigen-binding fragment competes for binding to LAYN with or binds the same LAYN epitope as antibody A1, A2, A85 or A86. Competition assays may be conducted, for example as described in PLOS One. 2014; 9(3):e92451 using a biosensor, or PLOS One 2020 □ar 5; 15(3):e0229206, or by a method disclosed herein.

### Production of Antibodies

For manufacturing the antibodies of the invention, the skilled artisan may choose from a variety of methods well known in the art.

For production of antibodies comprising two complete heavy and two complete light chains, like those of the IgG1 or IgG4 type, see Norderhaug et al., J Immunol Methods 1997, 204 (1): 77-87; Kipriyanow and Le Gall, Molecular Biotechnology 26:39-60, 2004; Shukla et al., 2007, J. Chromatography B, 848(1): 28-39.

In further aspects, the present invention provides a method of producing anti-LAYN antibodies of the invention and antigen-binding fragments thereof, comprising culturing a recombinant host cell expressing the heavy and light chains and isolating the antibody or antigen-binding fragment produced by the cell.

Preferably, the method for producing an antibody or antigen-binding fragment thereof comprises (a) culturing the host cell and (b) isolating the antibody or antigen-binding fragment thereof expressed from the cell. In addition, the method may optionally contain the step (c) of purifying the antibody or antigen-binding fragment.

An antibody of the invention can be produced by transfecting a host cell with one or more vectors comprising polynucleotides encoding the respective antibodies or fragments, culturing the host cell (in particular under conditions that allow synthesis of said antibody molecule); and recovering said antibody molecule from said culture. It is understood that the heavy and the light chain of an antibody may be encoded by separate polynucleotides. Accordingly, provided herein is also a plurality of polynucleotides encoding the (heavy and light chain of) the antibodies or antigen-binding fragments as described herein (by separate polynucleotides). Such a plurality of polynucleotides may be comprised in a (single) vector or in a plurality of vectors.

The antibody or antigen-binding fragment thereof (e.g. as monoclonal antibodies) can be made using recombinant DNA methods as described in U.S. Pat. No. 4,816,567, which is incorporated by reference herein.

The (isolated) polynucleotides encoding the heavy and light chains may be transfected into host cells such as E. coli cells, simian COS cells, Chinese hamster ovary (CHO) cells, or myeloma cells that do not otherwise produce immunoglobulin protein, monoclonal antibodies are generated by the host cells. For example, isolated polynucleotides encoding the heavy and light chains may be stably transfected into a suitable host cell, e.g., a CHO cell, for antibody production.

Various techniques are known for the production of antibody fragments.

Traditionally, these fragments are derived via proteolytic digestion of intact antibodies, as described, for example, by Morimoto et al., J. Biochem. Biophys. Meth. 24:107-117 (1993) and Brennan et al., Science 229:81 (1985). In some embodiments, anti-LAYN antibody fragments are produced recombinantly. Fab, Fv, and scFv antibody fragments can all be expressed in and secreted from E. coli or other host cells, thus allowing the production of large amounts of these fragments. Other techniques for the production of antibody fragments will be apparent to the skilled practitioner. For example, antibody fragments can be produced by techniques known in the art including, but not limited to: F(ab′)₂ fragment produced by pepsin digestion of an antibody molecule; Fab fragment generated by reducing the disulfide bridges of an F(ab′)₂ fragment; Fab fragment generated by the treatment of the antibody molecule with papain and a reducing agent. For example, Fab molecules may be produced by expression of nucleic acids encoding such constructs in host cells, like E. coli, Pichia pastoris, or mammalian cell lines (e.g. CHO, or NS0). Processes are known in the art which allow proper folding, association, and disulfide bonding of these chains into functional Fab molecules comprising a Fd fragment and a light chain (Burtet et al., J. Biochem. 2007, 142(6), 665-669; Quintero-Hernandez et al., Mol. Immunol. 2007, 44:1307-1315; Willems et al. J. Chromatogr. B. Analyt. Technol. Biomed. Life Sci. 2003; 786:161-176.). Processes for manufacturing scFv antibodies by recombinant expression of nucleic acids encoding scFv constructs in host cells (like E. coli, Pichia pastoris, or mammalian cell lines, e.g. CHO or NS0), yielding functional scFv molecules, are also known (Rippmann et al., Applied and Environmental Microbiology 1998, 64(12): 4862-4869; Yamawaki et al., J. Biosci. Bioeng. 2007, 104(5): 403-407; Sonoda et al., Protein Expr. Purif. 2010, 70(2): 248-253). Techniques can be adapted for the production of single-chain antibodies specific to LAYN (see, e.g., U.S. Pat. No. 4,946,778).

### Polynucleotides, Vectors, and Host Cells

The polynucleotides, vectors and host cells described in the following may be used in the production of the anti-LAYN antibodies, or antigen-binding fragments thereof, as described above.

The present disclosure also provides (isolated) polynucleotides that comprise a sequence encoding an anti-LAYN antibody or antigen-binding fragment thereof, vectors, and host cells comprising the polynucleotides, and recombinant techniques for production of the antibody. As described above, the antibody or antigen-binding fragment thereof, as described herein, may be encoded on a single or on a plurality of polynucleotides (e.g., with heavy and light chain encoded on separate polynucleotides). In some aspects, provided herein is a plurality of polynucleotides encoding the antibody according to the invention, or the antigen-binding fragment thereof. More specifically, said plurality of polynucleotides may comprise (or consist of):
(i) a first polynucleotide (comprising a nucleic acid sequence) encoding the heavy chain variable region (VH) or the heavy chain (HC) of an anti-LAYN antibody, or antigen-binding fragment thereof, as described herein (e.g., in Tables 2-8); and
(ii) a second polynucleotide (comprising a nucleic acid sequence) encoding the (corresponding) light chain variable region (VL) or the light chain (LC) of said anti-LAYN antibody, or antigen-binding fragment thereof, as described herein (e.g., in Tables 2-8).

The (isolated) polynucleotides can encode any desired form of the anti-LAYN antibody including, for example, full length monoclonal antibodies, Fab, Fab′, F(ab′)₂, and Fv fragments, diabodies, linear antibodies, single-chain antibody molecules, and multispecific antibodies formed from antibody fragments.

The polynucleotide(s) that comprise a sequence encoding an anti-LAYN antibody or a fragment or chain thereof can be fused to one or more regulatory or control sequence, as known in the art, and can be contained in suitable expression vectors or host cell as known in the art. Each of the polynucleotide molecules encoding the heavy or light chain variable domains can be independently fused to a polynucleotide sequence encoding a constant domain, such as a human constant domain, enabling the production of intact antibodies. Alternatively, polynucleotides, or portions thereof, can be fused together, providing a template for production of a single chain antibody.

For recombinant production, the (plurality of) polynucleotide encoding the antibody may be inserted into one or more replicable vector for cloning (amplification of the DNA) or for expression (expression vector). Many suitable vectors for expressing the recombinant antibodies are available. The vector components generally include, but are not limited to, one or more of the following: a signal sequence, an origin of replication, one or more marker genes, an enhancer element, a promoter, and a transcription termination sequence. Accordingly, the present disclosure provides a vector or plurality of vectors comprising a polynucleotide or a plurality of polynucleotides that encode the light chain and/or the heavy chain of an anti-LAYN antibody or antigen-binding fragment thereof as disclosed herein. Said vector may comprise an expression vector or plurality of expression vectors. Said polynucleotide or plurality of polynucleotides may be in functional association with one or more expression control sequences.

It is understood that the plurality of polynucleotides as described above, e.g. the first and second polynucleotides, may be introduced into separate vectors. Accordingly, the present invention also provides a plurality of vectors, e.g. a first and a second vector, comprising the plurality of polynucleotides, e.g. the first and the second polynucleotides, as described above.

In other words, the present disclosure also provides a vector (or a plurality of vectors) comprising the polynucleotide (or plurality of polynucleotides) encoding the anti-LAYN antibody, or the antigen-binding fragment thereof, as described herein (according to the present invention). In some embodiments, the plurality of vectors may comprise a first and a second vector, wherein the first vector comprises a first polynucleotide as described herein and the second vector comprises a second polynucleotide herein. A vector is usually a recombinant nucleic acid molecule. Accordingly, the vector may comprise one or more heterologous elements, such as a multiple cloning site, a (heterologous) promotor, a (heterologous) enhancer and/or a (heterologous) selection marker. The vector may be suitable for incorporating a polynucleotide sequence of interest. The vector may be a storage vector, an expression vector, a cloning vector, or a transfer vector. Preferably, the vector is an expression vector. In some embodiments, the vector comprises a cloning site, a selection marker, such as an antibiotic resistance factor, and/or a sequence suitable for multiplication of the vector, such as an origin of replication. The vector may be a plasmid vector.

Also included are nucleic acids that hybridize under low, moderate, and high stringency conditions, in particular under high stringency conditions, as defined herein, to all or a portion (e.g., the portion encoding the variable region) of the nucleotide sequence represented by isolated polynucleotide sequence(s) that encode an anti-LAYN antibody or antibody fragment. The hybridizing portion of the hybridizing nucleic acid is typically at least 15 (e.g., 20, 25, 30 or 50) nucleotides in length. The hybridizing portion of the hybridizing nucleic acid is at least 80%, e.g., at least 90%, at least 95%, at least 98%, or at least 99% identical to the sequence of a portion or all of a nucleic acid encoding an anti-LAYN polypeptide (e.g., a heavy chain or light chain variable region), or its complement. Hybridizing nucleic acids of the type described herein can be used, for example, as a cloning probe, a primer, e.g., a PCR primer, or a diagnostic probe. In some aspects, “high stringency conditions” means for probes of at least 100 nucleotides in length, prehybridization and hybridization at 42 °C in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65 °C.

In some embodiments, the present invention relates to an isolated polynucleotide comprising a nucleotide sequence encoding a heavy chain variable region comprising the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169, 181, 184, 186 or 188. Preferably, the isolated polynucleotide comprises a nucleotide sequence encoding a heavy chain variable region comprising the amino acid sequence of any one of SEQ ID NOs 7, 13, 17, 20, 22, 27, 30, 34, 78, 83, 86, 89, 92, 95, 97, 99, 101, 103, 105, 107, 109, 111, 113, 115, 117, 119, 121, 123, 126, 129, 132, 134, 136, 140, 143, 145, 169 or 181.

In some embodiments, the present invention relates to an isolated polynucleotide comprising a nucleotide sequence encoding a light chain variable region comprising the amino acid sequence of any one of SEQ ID NOs 8, 14, 25, 35, 39, 42, 45, 48, 51, 54, 56, 59, 62, 65, 68, 71, 73, 75, 79, 138, 170 or 179.

In some embodiments, the present invention relates to an isolated polynucleotide comprising a nucleotide sequence encoding a heavy chain comprising the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146, 182, 185, 187 or 189. Preferably, the isolated polynucleotide comprises a nucleotide sequence encoding a heavy chain comprising the amino acid sequence of any one of SEQ ID NOs 9, 15, 18, 21, 23, 28, 31, 36, 80, 84, 87, 90, 93, 96, 98, 100, 102, 104, 106, 108, 110, 112, 114, 116, 118, 120, 122, 124, 127, 130, 133, 135, 137, 141, 144, 146 or 182.

In some embodiments, the present invention relates to an isolated polynucleotide comprising the nucleotide sequence encoding of a light chain comprising the amino acid sequence of any one any one of SEQ NO: SEQ ID NOs 10, 16, 26, 37, 40, 43, 46, 49, 52, 55, 57, 60, 63, 66, 69, 72, 74, 76, 81, 139 or 180.

The anti-LAYN antibodies can also be produced as fusion polypeptides, in which the antibody is fused with a heterologous polypeptide, such as a signal sequence or other polypeptide having a specific cleavage site at the amino terminus of the mature protein or polypeptide. The heterologous signal sequence selected is typically one that is recognized and processed (e.g., cleaved by a signal peptidase) by the host cell. For prokaryotic host cells that do not recognize and process the anti-LAYN antibody signal sequence, the signal sequence can be substituted by a prokaryotic signal sequence. The signal sequence can be, for example, alkaline phosphatase, penicillinase, lipoprotein, heat-stable enterotoxin II leaders, and the like. For yeast secretion, the native signal sequence can be substituted, for example, with a leader sequence obtained from yeast invertase alpha-factor (including Saccharomyces and Kluyveromyces α-factor leaders), acid phosphatase, C. albicans glucoamylase, or the signal described in WO 90/13646. In mammalian cells, mammalian signal sequences as well as viral secretory leaders, for example, the herpes simplex gD signal, can be used. The DNA for such precursor region is ligated in reading frame to DNA encoding the anti-LAYN antibody.

Anti-LAYN antibody transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, or from heat-shock promoters, provided such promoters are compatible with the host cell systems.

Suitable host cells for cloning or expressing the DNA in the vectors herein are the prokaryote, yeast, or higher eukaryote cells as described aboveherein. Suitable prokaryotes for this purpose include eubacteria, such as Gram-negative or Gram-positive organisms, for example, Enterobacteriaceae such as Escherichia, e.g., E. coli. One preferred E. coli cloning host is E. coli 294 (ATCC 31,446), although other strains such as E. coli B, E. coli X₁₇₇₆ (ATCC 31,537), and E. coli W3110 (ATCC 27,325) are also suitable. These examples are illustrative rather than limiting.

In addition to prokaryotes, eukaryotic microbes such as filamentous fungi or yeast are suitable cloning or expression hosts for anti-LAYN antibody-encoding vectors. Saccharomyces cerevisiae, or common baker's yeast, is the most commonly used among lower eukaryotic host microorganisms.

Suitable host cells for the expression of glycosylated anti-LAYN antibody may also be derived from multicellular organisms. Examples of invertebrate cells include plant and insect cells, including, e.g., numerous baculoviral strains and variants and corresponding permissive insect host cells from hosts.

The anti-LAYN antibodies or antigen-binding fragments thereof can also be incorporated in viral vectors. For example, the polynucleotide encoding for the anti-LAYN antibody or antigen-binding fragment thereof may be introduced into the viral vector. The viral vector may then be used for expression of the anti-LAYN antibody, or antigen-binding fragment thereof, e.g., after administration of the viral vector to a host cell, a host organism or a patient.

The expression of the anti-LAYN antibody or antigen-binding fragment thereof may also be carried out in vertebrate cells. The propagation of vertebrate cells in culture (tissue culture) has become routine procedure and techniques are widely available. Preferably, the host cell (for expression of the anti-LAYN antibody or antigen-binding fragment) is a mammalian cell. Examples of useful mammalian host cell lines are monkey kidney CV1 line transformed by SV40 (COS-7, ATCC CRL 1651), human embryonic kidney line (HEK293 or 293 cells subcloned for growth in suspension culture, (Graham et al., 1977, J. Gen Virol. 36:59), baby hamster kidney cells (BHK, ATCC CCL 10), Chinese hamster ovary cells/-DHFR1 (CHO, Urlaub et al., 1980, Proc. Natl. Acad. Sci. USA 77:4216; e.g., DG44), mouse sertoli cells (TM4, Mather, 1980, Biol. Reprod. 23:243-252), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TR1 cells (Mather et al., 1982, Annals N.Y. Acad. Sci. 383:44-68), MRC 5 cells, FS4 cells, and human hepatoma line (Hep G2). More preferably, the host cell is a CHO cell or a Fut8-knockout cell. Specifically, the host cell may be a CHO-K1 FUT8^{-/-} cell. FUT8^{-/-} (Fut8-knockout) cells can produce afucosylated antibodies, which may be useful to increase ADCC.

### Compositions, Therapeutic Uses and Methods of Treatment

In some aspects, the present disclosure provides a pharmaceutical composition comprising the antibody, or an antigen-binding fragment thereof, according to the present invention and, optionally, a pharmaceutically acceptable carrier. The antibody molecule may be formulated into pharmaceutical compositions appropriate to facilitate administration to animals or humans, in particular for therapeutic uses. Typical formulations of the antibody molecule can be prepared by mixing the antibody molecule with physiologically acceptable carriers, diluents, excipients or stabilizers.

The pharmaceutical formulation may be in the form of lyophilized or otherwise dried formulations or aqueous solutions or aqueous or non-aqueous suspensions. Carriers, excipients, modifiers or stabilizers are typically nontoxic at the dosages and concentrations employed. Non-limiting examples of pharmaceutically acceptable carriers, diluents, excipients or stabilizers include buffer systems such as phosphate, citrate, acetate and other inorganic or organic acids and their salts; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; proteins, such as serum albumin, gelatin, or immunoglobulins; hydrophilic polymers such as polyvinylpyrrolidone or polyethylene glycol (PEG); amino acids such as glycine, glutamine, asparagine, histidine, arginine, or lysine; monosaccharides, disaccharides, oligosaccharides or polysaccharides and other carbohydrates including glucose, mannose, sucrose, trehalose, dextrins or dextrans; chelating agents such as EDTA (ethylenediaminetetraacetic acid); sugar alcohols such as, mannitol or sorbitol; salt-forming counter-ions such as sodium; metal complexes (e.g., Zn-protein complexes); and/or ionic or non-ionic surfactants such as TWEEN™ (polysorbates), PLURONICS™ or fatty acid esters, fatty acid ethers or sugar esters. Also organic solvents can be contained in the antibody formulation such as ethanol or isopropanol. The excipients may also have a release-modifying or absorption-modifying function.

In typical embodiments, the pharmaceutical composition is formulated in accordance with routine procedures as a pharmaceutical composition adapted for intravenous or subcutaneous administration to a subject. Typically, compositions for administration by injection are solutions in sterile isotonic aqueous buffer. In some embodiments, the pharmaceutical composition further comprises a solubilizing agent. Where necessary, the pharmaceutical composition can also include a local anesthetic, such as lignocaine, to ease pain at the site of the injection. Generally, the ingredients may be supplied either separately or mixed together in unit dosage form, for example, as a dry lyophilized powder or water free concentrate in a hermetically sealed container such as an ampoule or sachet indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

In some aspects, the present invention also provides pharmaceutical compositions administered as pharmaceutical compositions comprising a therapeutically effective amount of the anti-LAYN antibody or an antigen-binding fragment thereof, and optionally one or more additional therapeutic agents. Some aspects of the invention provide the use of one or more active ingredients in the manufacture of a medicament for the therapy of cancer, wherein said medicament comprises the anti-LAYN antibody or an antigen-binding fragment thereof as described herein.

Further, the pharmaceutical composition can be provided as a pharmaceutical kit comprising (a) a container containing an anti-LAYN antibody or an antigen-binding fragment thereof in lyophilized form and (b) a second container containing a pharmaceutically acceptable diluent (e.g., sterile water) for injection. The pharmaceutically acceptable diluent can be used for reconstitution or dilution of the lyophilized anti-LAYN antibody or antigen-binding fragment thereof.

Also provided herein is a kit comprising (a) a container containing an anti-LAYN antibody or an antigen-binding fragment thereof in lyophilized form and (b) a leaflet with information, e.g. regarding the use, administration and/or dosage regimen of the pharmaceutical composition.

In some aspects, the present disclosure provides the anti-LAYN antibodies of the invention or antigen-binding fragments thereof for use in medicine. In other words, the anti-LAYN antibodies or antigen-binding fragments thereof, as described herein, may be useful in medicine, in particular for treating and/or preventing cancer. Accordingly, the anti-LAYN antibodies of the invention or antigen-binding fragments thereof are useful as a medicament.

Also provided herein is the pharmaceutical composition comprising the anti-LAYN antibodies of the invention or antigen-binding fragments thereof for use in medicine. The below description with regard to therapeutic use and method of treatments of the anti-LAYN antibodies likewise applies to the pharmaceutical composition comprising the anti-LAYN antibody or antigen-binding fragment thereof.

Furthermore, the present invention relates to the use of an antibody of the invention for the treatment and/or prevention of a disease and/or condition wherein the activity of reducing or depleting LAYN-expressing cells is of therapeutic benefit.

The present invention provides antibodies for treatment (use in) of cancer. In general, the treatment may be therapeutic treatment (e.g., after the onset or diagnosis of a cancer) or prophylactic treatment (e.g., to reduce the risk of occurrence of a cancer).

In some embodiments, the cancer is a solid tumor. In some embodiments, the cancer is characterized by intratumoral regulatory T cells expressing LAYN. The antibodies of the invention can reduce or deplete regulatory T cells expressing LAYN. Further, the antibodies show favorable pharmacokinetics and/or pharmacodynamics, making them superior therapeutic agents for the treatment of a cancer, in particular cancer is characterized by intratumoral regulatory T cells expressing LAYN.

The anti-LAYN antibodies of the present invention can reduce or deplete intratumoral regulatory T cells. The present disclosure contemplates treating patients with a cancer, in particular a cancer characterized by intratumoral regulatory T cells expressing LAYN, with the anti-LAYN antibody or antigen-binding fragment thereof, as described herein, in particular to improve or stabilize the pathology, stabilize or decrease the size of the tumor, improve the quality of life, and/or to extend survival.

It has been shown that LAYN, in particular human LAYN isoform 2, is specifically expressed by intratumoral Tregs (De Simone et al., 2016, Immunity, 45(5): 1135-1147), while expression of LAYN is significantly lower in Tregs in healthy tissue. Without being bound to any theory, it is assumed that targeting intratumoral Tregs results in reduced side effects as compared to other cancer therapies, e.g., targeting Tregs in general. In some embodiments, the treatment with the anti-LAYN antibody or antigen-binding fragment thereof, as described herein, results in reduced side effects as compared to other cancer therapies.

In some embodiments, the antibodies of the invention (or an antigen-binding fragment thereof) are useful in therapeutic applications, to prevent or treat a cancer in a subject (or a patient). In some embodiments, the method comprises a method of treating and/or preventing cancer in a patient or subject, comprising administering to the patient in need thereof, a therapeutically effective amount of an antibody of the invention or an antigen-binding fragment thereof. In some embodiments, the cancer is a solid cancer. In some embodiments, the cancer is a cancer characterized by intratumoral Tregs expressing LAYN. Examples of cancers to be treated according to the invention include, but are not limited to, non-small cell lung cancer (NSCLC), head and neck squamous cell carcinoma (HNSCC), colorectal cancer (CRC), ovarian cancer (OC), breast cancer, renal cell carcinoma (RCC), hepatocellular carcinoma (HCC), gastric cancer, bladder cancer, pancreatic cancer and melanoma.

In some aspects, the invention provides antibodies for reducing the incidence of or preventing a cancer (e.g., as described herein) in a subject having a predisposition to develop such cancer. In some embodiments, the method comprises a method of reducing the incidence of and/or preventing a cancer in a patient or subject, comprising administering to the patient in need thereof, a therapeutically effective amount of an antibody of the invention. The anti-LAYN antibodies or antigen-binding fragments thereof, or the composition comprising an anti-LAYN antibody or an antigen-binding fragment thereof, according to the invention can be administered to a subject having or at risk of cancer, e.g., as described herein. The invention provides for the use of an anti-LAYN antibody or an antigen-binding fragment thereof in the manufacture of a medicament for prevention or treatment of a cancer.

For therapeutic applications, the antibodies of the invention may be administered to a mammal, preferably a human. The term “subject” as used herein means any mammalian patient to which an anti-LAYN antibody or an antigen-binding fragment thereof can be administered, including, e.g., humans and certain non-human mammals, such as domesticated animals (e.g., cows, sheep, pigs, cats, dogs, and horses), primates (e.g., humans and non-human primates such as monkeys), rabbits, and rodents (e.g., mice and rats). Subjects specifically intended for treatment using the methods described herein include humans.

Administration of the anti-LAYN antibody, or the antigen-binding fragment thereof, can be systemic or local. The preferred mode of application is parenteral, by infusion or injection (intravenous, intramuscular, subcutaneous, intraperitoneal, intradermal), but other modes of application such as by inhalation, transdermal, intranasal, buccal, oral, may also be applicable. In particular for the treatment of cancer (such as one or more of CRC, NSCLC, breast cancer, melanoma, HCC and HNSCC), the anti-LAYN antibody, or the antigen-binding fragment thereof, is preferably administered intravenously, e.g. by intravenous infusion. Formulations for injections may be prepared in, for example, prefilled syringes. As such in an aspect of the invention, pre-filled syringes are provided that include an anti-LAYN antibody or an antigen-binding fragment thereof.

For the prevention or treatment of disease, the appropriate dosage of antibody will depend on the type of disease to be treated, as defined above, the severity and course of the disease, whether the antibodies are administered for preventive or therapeutic purposes, the course of previous therapy, the patient's clinical history and response to the antibody, and the discretion of the attending physician. The dose range of the antibodies of the invention applicable per day is usually from 1 μg/kg to 100 mg/kg, preferably from 0.1 mg/kg to 20 mg/kg. The antibody is suitably administered to the patient at one time or over a series of treatments.

### Combination Therapy

The anti-LAYN antibody or an antigen-binding fragment thereof may be administered on their own (as stand-alone therapy) or in combination with one or more additional therapeutic agents. In some aspects of the invention, the anti-LAYN antibody or antigen-binding fragment thereof is optionally administered in combination with an additional therapeutic agent. The additional therapeutic agent may be state-of-the-art or standard-of-care compounds. In some embodiments, the additional therapeutic agent is an anti-cancer agent. Examples of additional therapeutic agents for combination therapy with the anti-LAYN antibody or an antigen-binding fragment thereof, as described herein, include, but are not limited to, steroids, immune modulators, immune checkpoint inhibitors, chemotherapeutic agents, cell proliferation inhibitors, anti-angiogenic substances, and cytostatic or cytotoxic substances.

With respect to therapeutic regimens for combinatorial administration, in a specific embodiment, an anti-LAYN antibody or antigen-binding fragment thereof is administered concurrently with a second (and/or third) therapeutic agent. In some embodiments, the second (and/or third) therapeutic agent is administered prior or subsequent to administration of the anti-LAYN antibody or antigen-binding fragment thereof.

Also provided herein are methods of treating or preventing or reducing the risk of occurrence of a cancer comprising administering (an effective amount of):
(a) the anti-LAYN antibody, or the antigen-binding fragment thereof (or a respective pharmaceutical formulation), as described herein; and
(b) a checkpoint inhibitor, e.g., a PD-1 antagonist, as described herein, (to a patient in need thereof).

The present disclosure also provides the anti-LAYN antibody, or the antigen-binding fragment thereof (or a respective pharmaceutical formulation), as described herein, for a therapeutic use as described above, e.g., for the treatment of a cancer, wherein the anti-LAYN antibody, or the fragment thereof, or the pharmaceutical composition is administered in combination with an immune checkpoint inhibitor. Accordingly, the anti-LAYN antibody, or the antigen-binding fragment thereof (or a respective pharmaceutical formulation), as described herein, maybe administered in combination with an immune checkpoint inhibitor.

As used herein, “combination” or “combined” within the meaning of this invention includes, without being limited, a product that results from the mixing or combining of more than one active agent and includes both fixed and non-fixed (e.g. free) combinations (including kits) and uses, such as e.g. the simultaneous, concurrent, sequential, successive, alternate or separate use of the components or agents. The term “fixed combination” means that the active agents are both administered to a patient simultaneously in the form of a single entity or dosage. The term “non-fixed combination” means that the active agents are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active agents.

In particular, “combination” or “combined treatment” usually means that the treatment with a first component or agent (e.g., the anti-LAYN antibody, or the antigen-binding fragment thereof (or a respective pharmaceutical formulation), as described herein), is combined with the treatment with a second component or agent (e.g., the additional therapeutic agent, such as the PD-1 antagonist, as described herein), wherein the first component or agent is different from the second component or agent. In some aspects, a “combination” or “combined treatment” means that the treatment schedule of the first component or agent is intertwined with the treatment schedule of the second component or agent. This may be even the case if the first and second component or agent are not administered at the same day.

The present disclosure also provides a kit comprising the anti-LAYN antibody, or the antigen-binding fragment thereof (or a respective pharmaceutical formulation), as described herein, and an immune checkpoint inhibitor, preferably a PD-1 antagonist.

As used herein, an “immune checkpoint inhibitor” (also referred to as “checkpoint inhibitor” or “inhibitor”) totally or partially reduces, inhibits, interferes with, or negatively modulates the function of one or more immune checkpoint molecules. Checkpoint molecules are known in the art. Checkpoint molecules, such as proteins, are typically involved in immune pathways and, for example, regulate T-cell activation, T-cell proliferation and/or T-cell function. Accordingly, the function of checkpoint inhibitors, which is negatively modulated (e.g., totally or partially reduced, inhibited or interfered with) by checkpoint inhibitors, is typically the (regulation of) T-cell activation, T-cell proliferation and/or T cell function. Immune checkpoint molecules thus regulate and maintain self-tolerance and the duration and amplitude of physiological immune responses. Many of the immune checkpoint molecules belong to the B7: CD28 family or to the tumor necrosis factor receptor (TNFR) super family and, by the binding of specific ligands, activate signaling molecules that are recruited to the cytoplasmic domain. Immune checkpoint inhibitors are typically able to modulate (i) self-tolerance and/or (ii) the amplitude and/or the duration of the immune response. Preferably, the immune checkpoint inhibitor used according to the present invention modulates the function of one or more human checkpoint molecules and is, thus, a “human checkpoint inhibitor”.

In some embodiments, the combination treatment with immune checkpoint inhibitors and the anti-LAYN antibody, or the antigen-binding fragment thereof (or a respective pharmaceutical formulation), as described herein, may be advantageous in tumors that are characterized by an activation of immune checkpoint pathways that suppress antitumor immune responses, thereby evading immunosurveillance. Typically, such tumors are characterized by an increased expression of the above immune checkpoint pathway genes, which may be detected by immunohistochemistry on tissue or tumor biopsies. For example, expression of PD-1 or PD-L1 may be determined using companion diagnostic kits, which utilize specific antibodies to assess PD-1 expression in tumor tissue in situ. Typically, the higher the number of PD-1 positive cells in a tumor the greater the likelihood that a patient afflicted with that tumor will respond to the combination treatment as described herein.

Preferably, the anti-LAYN antibody, or antigen-binding fragment thereof (or a pharmaceutical composition comprising the same), as described herein, is combined with a PD-1 antagonist. The present inventors have surprisingly found that the combination of the anti-LAYN antibody, or antigen-binding fragment thereof, and a PD-1 antagonist results in reduction of tumor growth and increased survival. In particular, the anti-LAYN antibody, or antigen-binding fragment thereof, and the PD-1 antagonist work together synergistically and can lead to a reduction of cancer.

PD-1 (programmed cell death-1) is known as an immunoinhibitory protein that negatively regulates TCR signals (Ishida, Y. et al. (1992) EMBO J. 11:3887-3895; Blank, C. et al. (2007) Immunol. Immunother. 56:739-745). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci USA 99:12293-7; Brown et al. (2003) J. Immunol. 170:1257-66). PD-1 is an inhibitory member of the extended CD28/CTLA-4 family of T cell regulators. Other members of the CD28 family include CD28, CTLA-4, ICOS and BTLA. PD-1 is suggested to exist as a monomer, lacking the unpaired cysteine residue characteristic of other CD28 family members. PD-1 is expressed on activated B cells, T cells, and monocytes (Okazaki et al. (2002) Curr Opin Immunol 14:779-82; Bennett et al. (2003) J. Immunol. 170:711-8). Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43). Both PD-L1 and PD-L2 are B7 homologs that bind to PD-1. PD-L1 is abundant in a variety of human cancers (Dong et al. (2002) Nat. Med. 8:793-800). The PD-1 gene encodes a 55 kDa type I transmembrane protein that is part of the Ig gene superfamily (Agata et al. (1996) Int Immunol. 8:765-72). The complete PD-1 sequence can be found under GenBank Accession No. U64863. Although structurally similar to CTLA-4, PD-1 lacks a sequence motif (MYPPY; SEQ ID NO: 219) that is important for B7-1 and B7-2 binding.

In recent years, monoclonal antibodies as PD-1 antagonists have been developed for use in therapy, more precisely for treating various diseases, including cancer and infectious diseases (e.g., WO2006/121168; WO2015/112900). Any one of such antibodies can be used according to the present invention.

As used herein, a “PD-1 antagonist” is a compound that reduces or inhibits the interaction of PD-1 with its receptor(s) or ligand(s). Accordingly, it may be an antagonist against a member of the protein Programmed Death 1 (PD-1) family, such as PD-1 itself or one of its ligands, PD-L1 or PD-L2. Preferably, the PD-1 antagonist is an antagonist or inhibitor of PD-1 or an antagonist or inhibitor of PD-L1. The PD-1 antagonist is preferably an anti-PD-1-antibody or an anti-PD-L1-antibody, and more preferably a humanized or fully human anti-PD-1 antibody or a humanized or fully human anti-PD-L1 antibody. Any one of these antibodies may be a recombinant human antibody.

PD-1 antagonists are well-known in the art, e.g. reviewed by Li et al., Int. J. Mol. Sci. 2016, 17, 1151 (incorporated herein by reference). Any PD-1 antagonist, especially antibodies, such as those disclosed by Li et al. as well as the further antibodies disclosed herein below, can be used according to the invention. Examples of PD-1 antagonists include, but are not limited to, pembrolizumab (also known as Keytruda lambrolizumab, MK-3475), nivolumab (Opdivo, MDX-1106, B MS-936558, ONO-4538), pidilizumab (CT-011), cemiplimab (Libtayo), ezabenlimab (BI754091), camrelizumab, AUNP12, AMP-224, AGEN-2034, BGB-A317 (Tisleizumab), spartalizumab (PDR001), MK-3477, SCH-900475, PF-06801591, JNJ-63723283, genolimzumab (CBT-501), LZM-009, BCD-100, SHR-1201, BAT-1306, AK-103 (HX-008), MEDI-0680 (also known as AMP-514) MEDI0608, JS001 (see Si-Yang Liu et al., J. Hematol. Oncol.10:136 (2017)), INCSHR1210 (also known as SHR-1210), TSR-042 (also known as ANB011), GLS-010 (also known as WBP3055), REGN2810, BMS-936559, AM-0001 (Armo), STI-1110 (see WO 2014/194302), AGEN2034 (see WO 2017/040790), MGA012 (see WO 2017/19846), IBI308 (see WO 2017/024465, WO 2017/025016, WO 2017/132825, and WO 2017/133540), monoclonal antibodies 5C4, 17D8, 2D3, 4H1, 4A11, 7D3, and 5F4, described in WO 2006/121168 and antibodies PD1-1, PD1-2, PD1-3, PD1-4, and PD1-5 as described in WO2017/198741.

Preferably, the PD-1 antagonist of this invention and all its embodiments is selected from the group consisting of pembrolizumab, nivolumab, ezabenlimab, pidilizumab, tislelizumab, spartalizumab, durvalumab, atezolizumab, avelumab, toripalimab, cemiplimab, camrelizumab, dostarlimab and cetrelimab. More preferably, the PD-1 antagonist is selected from the group consisting of the following antibodies:
pembrolizumab (anti-PD-1 antibody);
nivolumab (anti-PD-1 antibody);
cemiplimab (anti-PD-1 antibody);
ezabenlimab (anti-PD-1 antibody);
pidilizumab (anti-PD-1 antibody);
spartalizumab (anti-PD-1 antibody);
atezolizumab (anti-PD-L1 antibody);
avelumab (anti-PD-L1 antibody);
durvalumab (anti-PD-L1 antibody).

Pembrolizumab (formerly also known as lambrolizumab; trade name Keytruda; also known as MK-3475) is disclosed, e.g., in Hamid, O. et al. (2013) New England Journal of Medicine 369(2):134-44. It is a humanized IgG4 monoclonal antibody that binds to PD-1. It contains the mutation C228P, which is designed to prevent Fc-mediated cytotoxicity. Pembrolizumab is disclosed, e.g., in U.S. Pat. No. 8,354,509 and WO2009/114335. It is approved by the FDA for the treatment of patients suffering from unresectable or metastatic melanoma and patients with metastatic NSCLC.

Nivolumab (CAS Registry Number: 946414-94-4; B□S-936558 or MDX1106b) is a fully human IgG4 monoclonal antibody which specifically blocks PD-1, lacking detectable antibody-dependent cellular toxicity (ADCC). Nivolumab is, e.g., disclosed in U.S. Pat. No. 8,008,449 and WO2006/121168. It has been approved by the FDA for the treatment of patients suffering from unresectable or metastatic melanoma, metastatic NSCLC and advanced renal cell carcinoma.

Ezabenlimab is a mouse derived, humanized monoclonal IgG4Pro antibody (mAb) targeting human PD-1. Ezabenlimab potently blocks PD-1/PD-L1 and PD-1/PD-L2 interactions in vitro.

Pidilizumab (CT-011; Cure Tech) is a humanized IgG1k monoclonal antibody that binds to PD-1. Pidilizumab is, e.g., disclosed in WO2009/101611.

Spartalizumab (PDR-001 or PDR001) is a high-affinity, ligand-blocking, humanized anti-PD-1 IgG4 antibody that blocks the binding of PD-L1 and PD-L2 to PD-1. PDR-001 is disclosed in WO2015/112900 and WO2017/019896.

Atezolizumab (Tecentriq, also known as MPDL3280A) is a phage-derived human IgG1k monoclonal antibody targeting PD-L1 and is described e.g. in Deng et al. mAbs 2016; 8:593-603. It has been approved by the FDA for the treatment of patients suffering from urothelial carcinoma.

Avelumab (Bavencio, MSB0010718C) is a fully human anti-PD-L1 IgG1 monoclonal antibody and described in e.g. Boyerinas et al. Cancer Immunol. Res. 2015; 3:1148-1157.

Durvalumab (MEDI4736) is a human IgG1k monoclonal antibody with high specificity to PD-L1 and described in e.g. Stewart et al. Cancer Immunol. Res. 2015; 3:1052-1062 or in Ibrahim et al. Semin. Oncol. 2015; 42:474-483.

More preferably, the anti-PD-1 antibody is selected from the group consisting of pembrolizumab, nivolumab, cemiplimab, or PD1-1, PD1-2, PD1-3, PD1-4, and PD1-5 as described in WO2017/198741 (incorporated herein by reference), even more preferably said anti-PD-1 antibody is selected from pembrolizumab, nivolumab and ezabenlimab.

The INNs as used herein are meant to also encompass all biosimilar antibodies having the same, or substantially the same, amino acid sequences as the originator antibody, including but not limited to those biosimilar antibodies authorized under 42 USC § 262 subsection (k) in the US and equivalent regulations in other jurisdictions.

PD-1 antagonists listed above are known in the art with their respective manufacture, therapeutic use and properties.

In one embodiment the PD-1 antagonist is pembrolizumab.

In another embodiment the PD-1 antagonist is nivolumab.

In another embodiment the PD-1 antagonist is cemiplimab.

In another embodiment the PD-1 antagonist is ezabenlimab.

In another embodiment the PD-1 antagonist is pidilizumab.

In another embodiment the PD-1 antagonist is spartalizumab.

In another embodiment the PD-1 antagonist is atezolizumab.

In another embodiment the PD-1 antagonist is avelumab.

In another embodiment the PD-1 antagonist is durvalumab.

Administration of the PD-1 antagonist can be systemic or local. The preferred mode of application is parenteral, by infusion or injection (intravenous, intramuscular, subcutaneous, intraperitoneal, intradermal), but other modes of application such as by inhalation, transdermal, intranasal, buccal, oral, may also be applicable. Preferably, the PD-1 antagonist is administered intravenously, e.g. by intravenous infusion.

Within this invention it is to be understood that the combinations (combined use) according to this invention may envisage the simultaneous, concurrent, sequential, successive, alternate or separate administration of the active agents or components. It will be appreciated that (a) the anti-LAYN antibody, or the antigen-binding fragment thereof (or a respective pharmaceutical formulation), as described herein, and (b) the PD-1 antagonist can be administered formulated either dependently or independently. In particular, (a) the anti-LAYN antibody, or the antigen-binding fragment thereof (or a respective pharmaceutical formulation), as described herein, and (b) the PD-1 antagonist may be administered either as part of the same pharmaceutical composition/dosage form or, preferably, in separate pharmaceutical compositions/dosage forms. In some embodiments, such separate pharmaceutical compositions/dosage forms (of (a) and (b)) are administered concurrently, e.g. simultaneously.

The administration of (a) the anti-LAYN antibody, or the antigen-binding fragment thereof (or a respective pharmaceutical formulation), as described herein, and (b) the PD-1 antagonist may take place by co-administering the active components or agents, such as e.g. by administering them simultaneously or concurrently in one single or in two separate formulations or dosage forms. Alternatively, the administration of (a) the anti-LAYN antibody, or the antigen-binding fragment thereof (or a respective pharmaceutical formulation), as described herein, and (b) the PD-1 antagonist may take place by administering the active components or agents sequentially or in alteration, such as e.g. in two separate formulations or dosage forms.

For example, simultaneous administration includes administration at substantially the same time. This form of administration may also be referred to as “concomitant” administration. Concurrent administration includes administering the active agents within the same general time period, for example on the same day(s), but not necessarily at the same time. Alternate administration includes administration of one agent during a time period, for example over the course of a few days or a week, followed by administration of the other agent during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent during a second time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

Accordingly, in some embodiments, (a) the anti-LAYN antibody, or the antigen-binding fragment thereof (or a respective pharmaceutical formulation), as described herein, is administered simultaneously, concurrently, sequentially, successively, alternately or separately with (b) the PD-1 antagonist as described herein. In some embodiments, (b) the PD-1 antagonist as described herein, is administered simultaneously, concurrently, sequentially, successively, alternately or separately with (a) the anti-LAYN antibody, or the antigen-binding fragment thereof (or a respective pharmaceutical formulation), as described herein. In some aspects, the use of (a) the anti-LAYN antibody, or the antigen-binding fragment thereof (or a respective pharmaceutical formulation), as described herein, is provided wherein (a) the anti-LAYN antibody, or the antigen-binding fragment thereof (or a respective pharmaceutical formulation), as described herein, is to be administered simultaneously, concurrently, sequentially, successively, alternately or separately with (b) the PD-1 antagonist as described herein. In some aspects, the use of (b) the PD-1 antagonist as described herein is provided wherein (b) the PD-1 antagonist as described herein is to be administered simultaneously, concurrently, sequentially, successively, alternately or separately with (a) the anti-LAYN antibody, or the antigen-binding fragment thereof (or a respective pharmaceutical formulation), as described herein. In some aspects, a kit according to the present invention is provided wherein the first pharmaceutical composition (comprising either (a) the anti-LAYN antibody or the antigen-binding fragment thereof, or (b) the PD-1 antagonist as described herein) is to be administered simultaneously, concurrently, sequentially, successively, alternately or separately with the second pharmaceutical composition (comprising the other of (a) or (b), i.e. that is not comprised in the first pharmaceutical composition).

The compounds for combination as described herein may be administered at therapeutically effective amounts in single or divided doses administered at appropriate time intervals. A therapeutically effective amount refers to an amount effective at dosages and for periods of time necessary to achieve the desired therapeutic result and is the minimum amount necessary to prevent, ameliorate, or treat a disease or disorder. A therapeutically effective amount of the compounds according to the present invention may vary according to factors such as the disease state, age, sex, and weight of the individual, and the ability of the compound to elicit a desired response in the individual. A therapeutically effective amount is also one in which any toxic or detrimental effects of the compound is outweighed by the therapeutically beneficial effects. A therapeutically effective dose preferably inhibits a measurable parameter, e.g. a tumor growth rate by at least about 20%, more preferably by at least about 40%, even more preferably by at least about 60%, and still more preferably by at least about 80% relative to untreated subjects or relative to a preceding untreated period of the same subject that is to be treated.

The active compounds may be administered in such doses which are therapeutically effective in monotherapy, or in such doses which are lower or higher than the doses used in monotherapy, but when combined result in a desired (jointly) therapeutically effective amount. The amount of the anti-LAYN antibody, or the antigen-binding fragment thereof required for use in treatment may be adapted to the particular antibody selected, the route of administration, the nature of the condition being treated and the age and condition of the patient and will be ultimately at the discretion of the attendant physician or clinician. Also, the dosage of the antibody of the invention may be adapted depending on the target cell, tumor, tissue, graft, or organ.

The desired dose of (a) the anti-LAYN antibody, or the antigen-binding fragment thereof (or a respective pharmaceutical formulation), as described herein, or (b) the PD-1 antagonist, as described herein, may be administered as a fixed amount per administration or as bolus, to reach a set blood concentration in the patient.

The method, compounds, compounds for use, uses of compounds, pharmaceutical composition and kit according to the present invention comprises administering to the subject a combination of the anti-LAYN antibody, or the antigen-binding fragment thereof (or a respective pharmaceutical formulation), as described herein and an anti-PD-1 antibody molecule as described herein.

Depending on the cancerous disease to be treated, the treatment with the anti-LAYN antibody or the combination therapy as defined herein may be used on its own or in further combination with one or more additional therapeutic agents, in particular selected from chemotherapeutic agents or therapeutically active compounds that inhibit angiogenesis, signal transduction pathways or mitotic checkpoints in cancer cells.

The additional therapeutic agent may be administered simultaneously with, optionally as a component of the same pharmaceutical preparation, or before or after administration of the anti-LAYN antibody and/or (optionally) the PD1 antagonist.

The chemotherapeutic agent (for combination with the anti-LAYN antibody) may be selected from hormones, hormonal analogues and antihormonals (e.g. tamoxifen, toremifene, raloxifene, fulvestrant, megestrol acetate, flutamide, nilutamide, bicalutamide, cyproterone acetate, finasteride, buserelin acetate, fludrocortisone, fluoxymesterone, medroxyprogesterone, octreotide, arzoxifene, pasireotide, vapreotide), aromatase inhibitors (e.g. anastrozole, letrozole, liarozole, exemestane, atamestane, formestane), LHRH agonists and antagonists (e.g. goserelin acetate, leuprolide, abarelix, cetrorelix, deslorelin, histrelin, triptorelin), antimetabolites (e.g. antifolates like methotrexate, pemetrexed, pyrimidine analogues like 5 fluorouracil, capecitabine, decitabine, nelarabine, and gemcitabine, purine and adenosine analogues such as mercaptopurine thioguanine, cladribine and pentostatin, cytarabine, fludarabine); antitumor antibiotics (e.g. anthracyclines like doxorubicin, daunorubicin, epirubicin and idarubicin, mitomycin-C, bleomycin dactinomycin, plicamycin, mitoxantrone, pixantrone, streptozocin); platinum derivatives (e.g. cisplatin, oxaliplatin, carboplatin, lobaplatin, satraplatin); alkylating agents (e.g. estramustine, meclorethamine, melphalan, chlorambucil, busulphan, dacarbazine, cyclophosphamide, ifosfamide, hydroxyurea, temozolomide, nitrosoureas such as carmustine and lomustine, thiotepa); antimitotic agents (e.g. vinca alkaloids like vinblastine, vindesine, vinorelbine, vinflunine and vincristine; and taxanes like paclitaxel, docetaxel and their formulations, larotaxel; simotaxel, and epothilones like ixabepilone, patupilone, ZK-EPO); topoisomerase inhibitors (e.g. epipodophyllotoxins like etoposide and etopophos, teniposide, amsacrine, topotecan, irinotecan) and miscellaneous chemotherapeutics such as amifostine, anagrelide, interferone alpha, procarbazine, mitotane, and porfimer, bexarotene, celecoxib.

In some embodiments, the treatment involving (a) the anti-LAYN antibody, or the antigen-binding fragment thereof (or a respective pharmaceutical formulation), as described herein, and, optionally, (b) the PD-1 antagonist, as described herein, further includes a “platinum doublet” therapy, i.e. therapy with (i) a platinum compound such as cisplatin or carboplatin, plus (ii) a third-generation chemotherapy agent such as docetaxel, paclitaxel, vinorelbine, or gemcitabine.

### Non-Therapeutic Uses, Kits and Articles of Manufacture

The antibodies described herein are also useful as affinity purification agents. Accordingly, a method for affinity purification is provided herein, wherein the anti-LAYN antibodies are used. In affinity purification, the antibodies may be immobilized on a solid phase, such as Protein A resin, using methods well known in the art. The immobilized antibody may be contacted with a sample containing the LAYN protein (e.g., human LAYN isoform 1 or 2, or an ortholog thereof, or fragment thereof, such as the ECD of LAYN) to be purified, and thereafter the support is washed with a suitable solvent that will remove substantially all the material in the sample except the LAYN protein, which is bound to the immobilized antibody. Finally, the support is washed with another suitable solvent that will release the LAYN protein from the antibody.

The LAYN antibodies and fragments thereof, as disclosed herein. are also useful in diagnostic assays to detect and/or quantify LAYN protein, for example, detecting LAYN expression in specific cells, tissues, or plasma (e.g., of a (cancer) patient). Accordingly, the present disclosure also provides an (ex vivo or in-vitro) method of determining expression of LAYN using the anti-LAYN antibody, or an antigen-binding fragment thereof, as described herein. For example, the expression of LAYN may be determined in an isolated (cell or tissue) sample, e.g., from a (cancer) patient. Examples of such (isolated) samples include (cancer) tissue, blood, saliva, ascites fluid etc. Cancer tissue includes tissue of a tumor and or metastases.

In some embodiments, for example, for diagnostic purposes, the antibody may be labelled, e.g., with a detectable moiety. Numerous detectable labels are available, including radioisotopes, fluorescent labels, enzyme substrate labels, quantum dots and the like. The label may be directly or indirectly conjugated with the antibody using various known techniques. For example, the antibody can be conjugated with biotin and any of the categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody can be conjugated with a small hapten (such as digoxin) and one of the different types of labels mentioned above is conjugated with an anti-hapten antibody (e.g., anti-digoxin antibody). Thus, indirect conjugation of the label with the antibody can be achieved.

The anti-LAYN antibody or fragment thereof, as described herein, can also be used in a diagnostic kit, in particular in a packaged combination of reagents (in predetermined amounts), optionally, with instructions for performing the diagnostic assay. Examples of such assays include an assay for the detection of full length LAYN or the ECD of LAYN (e.g., human LAYN isoform 1 or 2, or an ortholog thereof, or fragment thereof, such as the ECD of LAYN), e.g., in a (isolated) sample (e.g., cancer tissue, blood, plasma). Accordingly, also provided herein is the use of the antibody or antigen-binding fragment thereof as described herein for diagnosis of a LAYN-associated disorder.

Where the antibody is labeled with an enzyme, the kit may include substrates and cofactors required by the enzyme such as a substrate precursor that provides the detectable chromophore or fluorophore. In addition, other additives may be included such as stabilizers, buffers (for example a block buffer or lysis buffer), and the like. The relative amounts of the various reagents may be varied widely to provide for concentrations in solution of the reagents that substantially optimize the sensitivity of the assay. The reagents may be provided as dry powders, usually lyophilized, including excipients that on dissolution will provide a reagent solution having the appropriate concentration.

In some aspects, the present disclosure provides an article of manufacture containing materials useful for the treatment of the disorders described above. The article of manufacture comprises a container and a label. Suitable containers include, for example, bottles, vials, syringes, and test tubes. The containers may be formed from a variety of materials such as glass or plastic. The container may hold a composition that is effective for treating the condition and may have a sterile access port. For example, the container may be an intravenous solution bag or a vial having a stopper pierceable by a hypodermic injection needle. The active agent in the composition is the anti-LAYN antibody or the antigen-binding fragment thereof. The label on or associated with the container indicates that the composition is used for treating the condition of choice. The article of manufacture may further comprise a second container comprising a pharmaceutically acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

### EXAMPLES

Other features and advantages of the subject technology will become apparent from the following more detailed Examples which illustrate, by way of example, the principles of the invention.

### Example 1: Cloning and Expression of LAYN Proteins

To identify an antibody, which binds specifically to LAYN expressed on intratumoral regulatory T cells (Tregs), different proteins based on the human extracellular domain (ECD) of human LAYN isoform 2 (also referred to as “LAYN-v2-His” and “LAYN-v2-no-tag”) were obtained:

SEQ ID NO: 190
RLLSGQPVCRGGTQRPCYKVIYFHDTSRRLNFEEAKEACRRDGGQL
VSIESEDEQKLIEKFIENLLPSDGDFWIGLRRREEKQSNSTACQDL
YAWTDGSISQFRNWYVDEPSCGSEVCVVMYHQPSAPAGIGGPYMFQ
WNDDRCNMKNNFICKYSDEKPAVPSREAEGEETELTTPVLPEETQE
EDAKKTFKESREAALNLAYGGHHHHHHHHHH
LAYN-v2-His: ECD of the human LAYN isoform 2,
GG-linker in bold and His-tag in italics.
SEQ ID NO: 191
RLLSGQPVCRGGTQRPCYKVIYFHDTSRRLNFEEAKEACRRDGGQL
VSIESEDEQKLIEKFIENLLPSDGDFWIGLRRREEKQSNSTACQDL
YAWTDGSISQFRNWYVDEPSCGSEVCVVMYHQPSAPAGIGGPYMFQ
WNDDRCNMKNNFICKYSDEKPAVPSREAEGEETELTTPVLPEETQE
EDAKKTFKESREAALNLAY
LAYN-v2-no-tag: ECD of the human LAYN isoform
2, no tag.

To increase the likelihood of identifying LAYN specific therapeutic antibodies that bind to the N-terminal part of LAYN, a shorter version (by 47 amino acids) of the ECD of human LAYN isoform 2 was used (referred to as “LAYN-v2-22-179-His”). The design of LAYN-v2-22-179-His was based on a structural model of LAYN protein, showing a flexible C-terminal domain of the ECD of LAYN which is situated close to the cell membrane according to the model and which is omitted in LAYN-v2-22-179-His.

SEQ ID NO: 192
RLLSGQPVCRGGTQRPCYKVIYFHDTSRRLNFEEAKEACRRDGGQL
VSIESEDEQKLIEKFIENLLPSDGDFWIGLRRREEKQSNSTACQDL
YAWTDGSISQFRNWYVDEPSCGSEVCVVMYHQPSAPAGIGGPYMFQ
WNDDRCNMKNNFICKYSDGGHHHHHHHHHH
LAYN v2 22-179-His: Truncated ECD of the human
LAYN isoform 2, GG-linker in bold and His-tag
in italics.

All LAYN ECD proteins were expressed in Expi293 or Jurkat cells and purified via Ni-NTA chromatography.

For stable transfection of HEK293 cells with full length human LAYN isoform 2, the following sequence (referred to as “FL-LAYN-SP v2”) was used:

SEQ ID NO: 193
MRPGTALQAVLLAVLLVGLRAATGRLLSGQPVCRGGTQRPCYKVIY
FHDTSRRLNFEEAKEACRRDGGQLVSIESEDEQKLIEKFIENLLPS
DGDFWIGLRRREEKQSNSTACQDLYAWTDGSISQFRNWYVDEPSCG
SEVCVVMYHQPSAPAGIGGPYMFQWNDDRCNMKNNFICKYSDEKPA
VPSREAEGEETELTTPVLPEETQEEDAKKTFKESREAALNLAYILI
PSIPLLLLLVVTTVVCWVWICRKRKREQPDPSTKKQHTIWPSPHQG
NSPDLEVYNVIRKQSEADLAETRPDLKNISFRVCSGEATPDDMSCD
YDNMAVNPSESGFVTLVSVESGFVTNDIYEFSPDQMGRSKESGWVE
NEIYGY
FL-LAYN-SP v2: human LAYN isoform 2 full
length, signal peptide in italics.

The mature protein does not include the signal peptide and, therefore, has the following sequence (referred to as “FL-LAYN v2”):

SEQ ID NO: 194
RLLSGQPVCRGGTQRPCYKVIYFHDTSRRLNFEEAKEACRRDGGQL
VSIESEDEQKLIEKFIENLLPSDGDFWIGLRRREEKQSNSTACQDL
YAWTDGSISQFRNWYVDEPSCGSEVCVVMYHQPSAPAGIGGPYMFQ
WNDDRCNMKNNFICKYSDEKPAVPSREAEGEETELTTPVLPEETQE
EDAKKTFKESREAALNLAYILIPSIPLLLLLVVTTVVCWVWICRKR
KREQPDPSTKKQHTIWPSPHQGNSPDLEVYNVIRKQSEADLAETRP
DLKNISFRVCSGEATPDDMSCDYDNMAVNPSESGFVTLVSVESGFV
TNDIYEFSPDQMGRSKESGWVENEIYGY
FL-LAYN v2: human LAYN isoform 2 full length,
no signal peptide

To generate genetically modified mice expressing human LAYN isoform 2 without the intracellular domain in FoxP3 positive regulatory T cells the following sequence (referred to as “LAYN v2 ECD1-250”) was used:

SEQ ID NO: 195
MRPGTALQAVLLAVLLVGLRAATGRLLSGQPVCRGGTQRPCYKVIY
FHDTSRRLNFEEAKEACRRDGGQLVSIESEDEQKLIEKFIENLLPS
DGDFWIGLRRREEKQSNSTACQDLYAWTDGSISQFRNWYVDEPSCG
SEVCVVMYHQPSAPAGIGGPYMFQWNDDRCNMKNNFICKYSDEKPA
VPSREAEGEETELTTPVLPEETQEEDAKKTFKESREAALNLAYILI
PSIPLLLLLVVTTVVCWVWI
LAYN v2 ECD1-250: human LAYN isoform 2 without
intracellular domain

In addition, human extracellular domain (ECD) of human LAYN isoform 1 (also referred to as “LAYN-v1-His”) was used in binding studies described herein below. LAYN-v1-His has the following sequence:

SEQ ID NO: 196
RLLSASDLDLRGGQPVCRGGTQRPCYKVIYFHDTSRRLNFEEAKEA
CRRDGGQLVSIESEDEQKLIEKFIENLLPSDGDFWIGLRRREEKQS
NSTACQDLYAWTDGSISQFRNWYVDEPSCGSEVCVVMYHQPSAPAG
IGGPYMFQWNDDRCNMKNNFICKYSDEKPAVPSREAEGEETELTTP
VLPEETQEEDAKKTFKESREAALNLAYGGHHHHHHHHHH
ECD of the human LAYN isoform 1, GG-linker in
bold and His-tag in italics.

For stable transfection of HEK293 cells in Example 22 with full length human LAYN isoform 1, the following sequence (referred to as “FL-LAYN-SP v1”) was used:

SEQ ID NO: 220
MRPGTALQAVLLAVLLVGLRAATGRLLSASDLDLRGGQPVCRGGTQ
RPCYKVIYFHDTSRRLNFEEAKEACRRDGGQLVSIESEDEQKLIEK
FIENLLPSDGDFWIGLRRREEKQSNSTACQDLYAWTDGSISQFRNW
YVDEPSCGSEVCVVMYHQPSAPAGIGGPYMFQWNDDRCNMKNNFIC
KYSDEKPAVPSREAEGEETELTTPVLPEETQEEDAKKTFKESREAA
LNLAYILIPSIPLLLLLVVTTVVCWVWICRKRKREQPDPSTKKQHT
IWPSPHQGNSPDLEVYNVIRKQSEADLAETRPDLKNISFRVCSGEA
TPDDMSCDYDNMAVNPSESGFVTLVSVESGFVTNDIYEFSPDQMGR
SKESGWVENEIYGY
FL-LAYN-SP v1: human LAYN isoform 1 full
length, signal peptide in italics.

For stable transfection of HEK293 cells in Example 22 with full length human LAYN isoform 3, the following sequence (referred to as “FL-LAYN-SP v3”) was used:

SEQ ID NO: 221
MVTSGLGSGGVRRNKAIAQPARTFMLGLMAAYHNLEKPAVPSREAE
GEETELTTPVLPEETQEEDAKKTFKESREAALNLAYILIPSIPLLL
LLVVTTVVCWVWICRKRKREQPDPSTKKQHTIWPSPHQGNSPDLEV
YNVIRKQSEADLAETRPDLKNISFRVCSGEATPDDMSCDYDNMAVN
PSESGFVTLVSVESGFVTNDIYEFSPDQMGRSKESGWVENEIYGY
FL-LAYN-SP v3: human LAYN isoform 3 full
length, predicted putative signal peptide in
italics.

For stable transfection of HEK293 cells in Example 22 with full length human LAYN isoform 4, the following sequence (referred to as “FL-LAYN-SP v4”) was used:

SEQ ID NO: 222
MYHQPSAPAGIGGPYMFQWNDDRCNMKNNFICKYSDEKPAVPSREA
EGEETELTTPVLPEETQEEDAKKTFKESREAALNLAYILIPSIPLL
LLLVVTTVVCWVWICRKRKREQPDPSTKKQHTIWPSPHQGNSPDLE
VYNVIRKQSEADLAETRPDLKNISFRVCSGEATPDDMSCDYDNMAV
NPSESGFVTLVSVESGFVTNDIYEFSPDQMGRSKESGWVENEIYGY
FL-LAYN-SP v4: human LAYN isoform 4 full
length, predicted putative signal peptide in
italics.

### Example 2: Identification of Anti-LAYN Antibodies

BALB/c mice were immunized with LAYN v2 22-179-His (SEQ ID NO: 192). This sequence is only included in human LAYN isoforms 1, 2 and partially in isoform 4, while human LAYN isoform 3 does not include this sequence (see SEQ ID NO: 192 and FIG. 1A). Therefore, it is expected that antibodies raised against this sequence are specific for LAYN isoforms 1 and 2.

B cells were harvested and used to produce hybridomas by fusion with myeloma cells. To select the most suitable monoclonal antibodies (mAbs), the following screening cascade was used, starting with the screening of hybridoma supernatants using an Enzyme-Linked Immunosorbent Assay (ELISA) that employed the use of recombinant LAYN ECD. The coating antigen was LAYN-v2-22-179-His, applied at a concentration of 1 μg/ml, with a volume of 100 μl per well. The coating buffer used was Phosphate Buffered Saline (PBS), with a pH of 7.4. The secondary antibody utilized was Peroxidase-AffiniPure Goat Anti-Mouse IgG. Seven mouse clones were selected for further testing.

The selected purified hybridoma supernatants underwent further testing. ELISA was performed on LAYN-v2-His (SEQ ID NO: 190) coated at 500 ng/well in PBS+/−5 mM TCEP. Blocking was done with 5% BSA in PBS. The antibodies were tested at single concentration of 10 μg/ml. All washes were performed with PBS with 0.05% Tween. For detection goat anti-mouse HRP (Invitrogen 31340) was used. Absorption was measured at A450 in triplicates. A specific signal of 2.33 was observed for LAYN-v2-His without TCEP, whereas for TCEP treated LAYN-v2-His an absorption A450=0.16 was observed.

Binding on Hek293 cells transfected with FL-LAYN-SP v2 (SEQ ID NO: 193) was confirmed with purified mouse hybridoma supernatants 24 h post transfection. Antibodies were titrated from 2 μg/ml down to 0.000128 μg/ml (7 points, 5-fold dilutions). Data fitting was performed on MFI data using GraphPad Prism and revealed an EC50 of 0.32 nM.

Additionally, the supernatants were tested for positive binding to LAYN on intratumoral T regulatory cells (Tregs). To this end, a tissue sample obtained from a colorectal cancer (CRC) patient was used. Staining was performed on the CRC sample comparing Tregs from tumor and adjacent healthy tissue. The purified antibodies were therefore labeled with Alexa Fluor 488 and used at a concentration of 5 μg/ml in the staining. LAYN % positivity was calculated using total Tregs (gated as: CD4+/CD127−/CD25+) as parental population. For Antibody P, e.g., 8.6% LAYN+ cells were detected in the analyzed CRC sample.

Based on these experiments, antibody P was selected, because it demonstrated binding to LAYN-v2-His in the ELISA as well as binding to intratumoral Tregs and LAYN-transfected HEK293 cells. Antibody P was sequenced from hybridoma using standard techniques. The following sequences were obtained for antibody P (Table 9):

TABLE 9
Sequences (SEQ ID NOs) of the six CDRs (according to the numbering
scheme of Kabat), heavy chain variable region (VH), light chain variable
region (VL), (full length) heavy chain (HC) and (full length) light
chain (LC) of the murine parental antibody (Antibody P)

| | Heavy chain | | | | Light chain | | | |
|---|---|---|---|---|---|---|---|---|
| Antibody P | CDR1 | CDR2 | CDR3 | VH | CDR1 | CDR2 | CDR3 | VL |
| SEQ ID NO. | 1 | 168 | 142 | 169 | 24 | 5 | 6 | 170 |

The sequences of antibody P were then humanized and, optionally, further optimized. Thereby, humanized antibodies A1-A104 were obtained (see Table 10 below). Briefly, the optimization included removing a predicted isomerization site (D107), removing predicted T cell epitopes and improvement or maintenance of biophysical properties and humanness. Sequence optimization also involved removing potential liabilities, such as in silico predicted deamidation sites, aspartate isomerization sites and oxidation residues that may be points of process control later in manufacturing, while maintaining binding to human LAYN. The resulting VH and VL sequences were formatted as hIgG1 (G1m3 allotype) having the constant region IgG1 CH1-CH2-CH3 sequence of SEQ ID NO: 173 and IgG1 CL sequence of SEQ ID NO: 174, respectively. Accordingly, the heavy (HC) and light chain (LC) sequences provided herein for the exemplary antibodies contain (i) the respective variable region sequences (VH or VL, as indicated) and (ii) the constant region sequences (CH1-CH2-CH3 or CL) of SEQ ID NOs 173 and 174, respectively, unless indicated otherwise.

TABLE 10
Sequences (SEQ ID NOs) of the six CDRs (according to the numbering scheme
of Kabat), heavy chain variable region (VH), light chain variable region
(VL), (full length) heavy chain (HC) and (full length) light chain (LC)
of each of antibodies A1-A104. Antibodies A01, A02, A03, A04, A05, A06,
A07, A08 and A09 are also referred to herein (i.e., in the entire specification)
as antibodies A1, A2, A3, A4, A5, A6, A7, A8 and A9, respectively.

| | Heavy chain SEQ ID NOs | | | | | Light chain SEQ ID NOs | | | | |
|---|---|---|---|---|---|---|---|---|---|---|
| mAb | CDR1 | CDR2 | CDR3 | VH | HC | CDR1 | CDR2 | CDR3 | VL | LC |
| A01 | 1 | 2 | 3 | 7 | 9 | 4 | 5 | 6 | 8 | 10 |
| A02 | 1 | 11 | 3 | 13 | 15 | 12 | 5 | 6 | 14 | 16 |
| A03 | 1 | 2 | 3 | 17 | 18 | 4 | 5 | 6 | 8 | 10 |
| A04 | 1 | 19 | 3 | 20 | 21 | 12 | 5 | 6 | 14 | 16 |
| A05 | 1 | 19 | 3 | 22 | 23 | 4 | 5 | 6 | 8 | 10 |
| A06 | 1 | 19 | 3 | 22 | 23 | 12 | 5 | 6 | 14 | 16 |
| A07 | 1 | 11 | 3 | 13 | 15 | 24 | 5 | 6 | 25 | 26 |
| A08 | 1 | 19 | 3 | 27 | 28 | 4 | 5 | 6 | 8 | 10 |
| A09 | 1 | 19 | 3 | 27 | 28 | 12 | 5 | 6 | 14 | 16 |
| A10 | 1 | 19 | 29 | 30 | 31 | 4 | 5 | 6 | 8 | 10 |
| A11 | 1 | 19 | 29 | 30 | 31 | 12 | 5 | 6 | 14 | 16 |
| A12 | 1 | 19 | 32 | 34 | 36 | 24 | 5 | 33 | 35 | 37 |
| A13 | 1 | 19 | 32 | 34 | 36 | 24 | 5 | 38 | 39 | 40 |
| A14 | 1 | 19 | 32 | 34 | 36 | 24 | 5 | 41 | 42 | 43 |
| A15 | 1 | 19 | 32 | 34 | 36 | 24 | 5 | 6 | 25 | 26 |
| A16 | 1 | 19 | 32 | 34 | 36 | 24 | 44 | 6 | 45 | 46 |
| A17 | 1 | 19 | 32 | 34 | 36 | 24 | 47 | 6 | 48 | 49 |
| A18 | 1 | 19 | 32 | 34 | 36 | 24 | 50 | 6 | 51 | 52 |
| A19 | 1 | 19 | 32 | 34 | 36 | 24 | 53 | 6 | 54 | 55 |
| A20 | 1 | 19 | 32 | 34 | 36 | 24 | 5 | 6 | 56 | 57 |
| A21 | 1 | 19 | 32 | 34 | 36 | 58 | 5 | 6 | 59 | 60 |
| A22 | 1 | 19 | 32 | 34 | 36 | 61 | 5 | 6 | 62 | 63 |
| A23 | 1 | 19 | 32 | 34 | 36 | 64 | 5 | 6 | 65 | 66 |
| A24 | 1 | 19 | 32 | 34 | 36 | 67 | 5 | 6 | 68 | 69 |
| A25 | 1 | 19 | 32 | 34 | 36 | 70 | 5 | 6 | 71 | 72 |
| A26 | 1 | 19 | 32 | 34 | 36 | 12 | 5 | 6 | 73 | 74 |
| A27 | 1 | 19 | 32 | 34 | 36 | 24 | 5 | 6 | 75 | 76 |
| A28 | 1 | 19 | 77 | 78 | 80 | 24 | 5 | 6 | 79 | 81 |
| A29 | 1 | 19 | 82 | 83 | 84 | 24 | 5 | 6 | 79 | 81 |
| A30 | 1 | 19 | 3 | 27 | 28 | 24 | 5 | 6 | 79 | 81 |
| A31 | 1 | 19 | 85 | 86 | 87 | 24 | 5 | 6 | 79 | 81 |
| A32 | 1 | 19 | 88 | 89 | 90 | 24 | 5 | 6 | 79 | 81 |
| A33 | 1 | 19 | 91 | 92 | 93 | 24 | 5 | 6 | 79 | 81 |
| A34 | 1 | 19 | 94 | 95 | 96 | 24 | 5 | 6 | 79 | 81 |
| A35 | 1 | 19 | 32 | 97 | 98 | 24 | 5 | 6 | 79 | 81 |
| A36 | 1 | 19 | 32 | 99 | 100 | 24 | 5 | 6 | 79 | 81 |
| A37 | 1 | 19 | 32 | 101 | 102 | 24 | 5 | 6 | 79 | 81 |
| A38 | 1 | 19 | 32 | 103 | 104 | 24 | 5 | 6 | 79 | 81 |
| A39 | 1 | 19 | 32 | 105 | 106 | 24 | 5 | 6 | 79 | 81 |
| A40 | 1 | 19 | 32 | 107 | 108 | 24 | 5 | 6 | 79 | 81 |
| A41 | 1 | 19 | 32 | 109 | 110 | 24 | 5 | 6 | 79 | 81 |
| A42 | 1 | 19 | 32 | 111 | 112 | 24 | 5 | 6 | 79 | 81 |
| A43 | 1 | 19 | 32 | 113 | 114 | 24 | 5 | 6 | 79 | 81 |
| A44 | 1 | 19 | 32 | 115 | 116 | 24 | 5 | 6 | 79 | 81 |
| A45 | 1 | 19 | 32 | 117 | 118 | 24 | 5 | 6 | 79 | 81 |
| A46 | 1 | 19 | 32 | 119 | 120 | 24 | 5 | 6 | 79 | 81 |
| A47 | 1 | 19 | 32 | 121 | 122 | 24 | 5 | 6 | 79 | 81 |
| A48 | 1 | 11 | 32 | 123 | 124 | 24 | 5 | 6 | 79 | 81 |
| A49 | 1 | 125 | 32 | 126 | 127 | 24 | 5 | 6 | 79 | 81 |
| A50 | 1 | 128 | 32 | 129 | 130 | 24 | 5 | 6 | 79 | 81 |
| A51 | 131 | 19 | 32 | 132 | 133 | 24 | 5 | 6 | 79 | 81 |
| A52 | 1 | 19 | 32 | 134 | 135 | 24 | 5 | 6 | 79 | 81 |
| A53 | 1 | 19 | 32 | 136 | 137 | 24 | 5 | 6 | 79 | 81 |
| A54 | 1 | 19 | 32 | 34 | 36 | 4 | 5 | 6 | 8 | 10 |
| A55 | 1 | 19 | 32 | 34 | 36 | 12 | 5 | 6 | 14 | 16 |
| A56 | 1 | 19 | 32 | 34 | 36 | 12 | 5 | 6 | 138 | 139 |
| A57 | 1 | 2 | 3 | 17 | 18 | 24 | 5 | 6 | 79 | 81 |
| A58 | 1 | 2 | 3 | 17 | 18 | 12 | 5 | 6 | 14 | 16 |
| A59 | 1 | 2 | 3 | 17 | 18 | 12 | 5 | 6 | 138 | 139 |
| A60 | 1 | 2 | 3 | 17 | 18 | 24 | 5 | 6 | 25 | 26 |
| A61 | 1 | 2 | 3 | 7 | 9 | 24 | 5 | 6 | 79 | 81 |
| A62 | 1 | 2 | 3 | 7 | 9 | 12 | 5 | 6 | 14 | 16 |
| A63 | 1 | 2 | 3 | 7 | 9 | 12 | 5 | 6 | 138 | 139 |
| A64 | 1 | 2 | 3 | 7 | 9 | 24 | 5 | 6 | 25 | 26 |
| A65 | 1 | 125 | 3 | 140 | 141 | 24 | 5 | 6 | 79 | 81 |
| A66 | 1 | 125 | 3 | 140 | 141 | 4 | 5 | 6 | 8 | 10 |
| A67 | 1 | 125 | 3 | 140 | 141 | 12 | 5 | 6 | 14 | 16 |
| A68 | 1 | 125 | 3 | 140 | 141 | 12 | 5 | 6 | 138 | 139 |
| A69 | 1 | 125 | 3 | 140 | 141 | 24 | 5 | 6 | 25 | 26 |
| A70 | 1 | 19 | 3 | 20 | 21 | 24 | 5 | 6 | 79 | 81 |
| A71 | 1 | 19 | 3 | 20 | 21 | 4 | 5 | 6 | 8 | 10 |
| A72 | 1 | 19 | 3 | 20 | 21 | 12 | 5 | 6 | 138 | 139 |
| A73 | 1 | 19 | 3 | 20 | 21 | 24 | 5 | 6 | 25 | 26 |
| A74 | 1 | 19 | 3 | 22 | 23 | 24 | 5 | 6 | 79 | 81 |
| A75 | 1 | 19 | 3 | 22 | 23 | 12 | 5 | 6 | 138 | 139 |
| A76 | 1 | 19 | 3 | 22 | 23 | 24 | 5 | 6 | 25 | 26 |
| A77 | 1 | 11 | 3 | 13 | 15 | 24 | 5 | 6 | 79 | 81 |
| A78 | 1 | 11 | 3 | 13 | 15 | 4 | 5 | 6 | 8 | 10 |
| A79 | 1 | 11 | 3 | 13 | 15 | 12 | 5 | 6 | 138 | 139 |
| A80 | 1 | 19 | 3 | 27 | 28 | 12 | 5 | 6 | 138 | 139 |
| A81 | 1 | 19 | 3 | 27 | 28 | 24 | 5 | 6 | 25 | 26 |
| A82 | 1 | 19 | 29 | 30 | 31 | 24 | 5 | 6 | 79 | 81 |
| A83 | 1 | 19 | 29 | 30 | 31 | 12 | 5 | 6 | 138 | 139 |
| A84 | 1 | 19 | 29 | 30 | 31 | 24 | 5 | 6 | 25 | 26 |
| A85 | 1 | 19 | 142 | 143 | 144 | 24 | 5 | 6 | 79 | 81 |
| A86 | 1 | 19 | 32 | 34 | 36 | 24 | 5 | 6 | 79 | 81 |
| A87 | 1 | 19 | 142 | 145 | 146 | 24 | 5 | 6 | 79 | 81 |
| A88 | 1 | 19 | 142 | 181 | 182 | 24 | 5 | 6 | 79 | 81 |
| A89 | 1 | 19 | 142 | 143 | 144 | 24 | 178 | 6 | 179 | 180 |
| A90 | 1 | 19 | 142 | 145 | 146 | 24 | 178 | 6 | 179 | 180 |
| A91 | 1 | 19 | 142 | 181 | 182 | 24 | 178 | 6 | 179 | 180 |
| A92 | 1 | 19 | 183 | 184 | 185 | 24 | 5 | 6 | 79 | 81 |
| A93 | 1 | 19 | 183 | 186 | 187 | 24 | 5 | 6 | 79 | 81 |
| A94 | 1 | 19 | 183 | 188 | 189 | 24 | 5 | 6 | 79 | 81 |
| A95 | 1 | 19 | 183 | 184 | 185 | 24 | 178 | 6 | 179 | 180 |
| A96 | 1 | 19 | 183 | 186 | 187 | 24 | 178 | 6 | 179 | 180 |
| A97 | 1 | 19 | 183 | 188 | 189 | 24 | 178 | 6 | 179 | 180 |
| A98 | 1 | 19 | 32 | 34 | 36 | 24 | 5 | 147 | 148 | 149 |
| A99 | 1 | 19 | 150 | 151 | 152 | 24 | 5 | 6 | 79 | 81 |
| A100 | 1 | 19 | 153 | 154 | 155 | 24 | 5 | 6 | 79 | 81 |
| A101 | 1 | 156 | 32 | 157 | 158 | 24 | 5 | 6 | 79 | 81 |
| A102 | 1 | 159 | 32 | 160 | 161 | 24 | 5 | 6 | 79 | 81 |
| A103 | 162 | 19 | 32 | 163 | 164 | 24 | 5 | 6 | 79 | 81 |
| A104 | 165 | 19 | 32 | 166 | 167 | 24 | 5 | 6 | 79 | 81 |

FIG. 2 shows an alignment of (A) VH sequences and (B) VL sequences of antibodies P and A1-A88, A90 and A91. Based on the alignment of the exemplary anti-LAYN antibodies, a respective consensus sequence for VH (FIG. 2A) and VL (FIG. 2B) was obtained, which is shown in SEQ ID NO: 212 (Consensus VH) and SEQ ID NO: 213 (Consensus VL). Boxes indicate CDR sequences according to different numbering schemes (Kabat, Aho, CCG, Chothia and IMGT).

In the following Examples/experimental data, antibodies A1-A12 were used as afucosylated antibodies, unless explicitly indicated otherwise. Afucosylation of the antibodies is described in more detail in Example 8.

### Example 3: Characterization of Anti-LAYN Antibodies: %-Humanness and Binding Activity

The antibodies were characterized with regard to %-humanness and binding activity was evaluated by surface plasmon resonance (SPR).

Briefly, %-humanness was calculated by comparing VH and VL separately to the closest human germline sequence using only the variable regions. In this context, the VL germline sequence used was IGKV1-12*01|human including the J region IGKJ4*01 (SEQ ID NO: 224); and the VH germline sequence used was IGHV1-46*03|human including the J region IGHJ4*01 (SEQ ID NO: 225). In the case of antibody A85 both heavy and light J regions were 100% identical to the corresponding human J regions. Total % humanness was calculated as (VH %+VL %)/2. Results are shown in Table 11A below.

For SPR, the affinities of the antibodies described above to LAYN-v2-His (SEQ ID NO: 190) were determined on a Biacore 8K+ instrument using HBS-EP+ (Cytiva #BR-100668) as running buffer at 25 °C. Following manufacturer's recommendations, a CM5 biosensor chip (Cytiva #BR100530) was activated with a 50:50 solution of EDC/NHS (amine coupling kit Cytiva #BR-100633). Protein A/G at 10 μg/mL in 10 mM acetate pH 4.5 was immobilized on the surface followed by deactivation with 1 M ethanolamine HCl. Anti LAYN antibodies were captured at 2 μg/mL in HBS-EP+ for 30 seconds at a flowrate of 10 L/min. Serial dilutions of LAYN-v2-His were prepared in HBS-EP+ (100 nM-3.13 nM) and injected for 120 seconds association at 30 μL/min followed by a dissociation of 180 seconds. The surface was regenerated by injecting 0.85% phosphoric acid for 30 sec at a flowrate of 30 μL/min. The analyte interaction with sensor surface (flow cell 1) and blank (HBS-EP+ or 0 nM analyte) were subtracted from the raw data. Sensorgrams were then fit using 1:1 binding model to provide affinity (K_{D}) values.

SPR was performed at pH 7.4 and at pH 6 to compare binding properties of the anti-LAYN antibodies at neutral and slightly acidic pH, which is commonly found in tumor microenvironment due to the accumulation of acidic metabolic waste products (Boedtkjer E, Pedersen S F. The Acidic Tumor Microenvironment as a Driver of Cancer. Annu Rev Physiol. 2020 Feb. 10; 82:103-126. doi: 10.1146/annurev-physiol-021119-034627. Epub 2019 Nov. 15). The same experimental setup was used as described above but using PBS-P adjusted to pH 7.4 or pH 6 as a running buffer instead of HBS-EP+. Results are shown in Table 11B below.

TABLE 11A
Results for % humanness

| | % total humaness | % Humanness | % Humanness |
|---|---|---|---|
| Antibody | (including J-region) | VH | VL |
| A01 | 92.04 | 90.76 | 93.46 |
| A02 | 89.82 | 88.24 | 91.59 |
| A03 | 92.48 | 91.6 | 93.46 |
| A04 | 89.82 | 88.24 | 91.59 |
| A05 | 91.15 | 89.08 | 93.46 |
| A06 | 90.27 | 89.08 | 91.59 |
| A07 | 88.94 | 88.24 | 89.72 |
| A08 | 90.27 | 87.39 | 93.46 |
| A09 | 89.38 | 87.39 | 91.59 |
| A10 | 90.27 | 87.39 | 93.46 |
| A11 | 89.38 | 87.39 | 91.59 |
| A12 | 89.0 | 88.2 | 89.7 |
| A13 | 89.0 | 88.2 | 89.7 |
| A14 | 88.5 | 88.2 | 88.8 |
| A15 | 88.94 | 88.24 | 89.72 |
| A16 | 88.5 | 88.2 | 88.8 |
| A17 | 89.0 | 88.2 | 89.7 |
| A18 | 89.0 | 88.2 | 89.7 |
| A19 | 89.0 | 88.2 | 89.7 |
| A20 | 89.0 | 88.2 | 89.7 |
| A21 | 88.0 | 88.2 | 87.9 |
| A22 | 89.0 | 88.2 | 89.7 |
| A23 | 89.0 | 88.2 | 89.7 |
| A24 | 89.0 | 88.2 | 89.7 |
| A25 | 88.5 | 88.2 | 88.8 |
| A26 | 89.0 | 88.2 | 89.7 |
| A27 | 88.0 | 88.2 | 87.9 |
| A28 | 88.1 | 87.4 | 88.8 |
| A29 | 88.1 | 87.4 | 88.8 |
| A30 | 88.05 | 87.39 | 88.79 |
| A31 | 88.1 | 87.4 | 88.8 |
| A32 | 88.1 | 87.4 | 88.8 |
| A33 | 88.1 | 87.4 | 88.8 |
| A34 | 88.1 | 87.4 | 88.8 |
| A35 | 88.9 | 89.1 | 88.8 |
| A36 | 88.1 | 87.4 | 88.8 |
| A37 | 88.1 | 87.4 | 88.8 |
| A38 | 88.5 | 88.2 | 88.8 |
| A39 | 88.9 | 89.1 | 88.8 |
| A40 | 88.1 | 87.4 | 88.8 |
| A41 | 88.1 | 87.4 | 88.8 |
| A42 | 88.9 | 89.1 | 88.8 |
| A43 | 88.5 | 88.2 | 88.8 |
| A44 | 88.9 | 89.1 | 88.8 |
| A45 | 88.5 | 88.2 | 88.8 |
| A46 | 88.9 | 89.1 | 88.8 |
| A47 | 88.9 | 89.1 | 88.8 |
| A48 | 88.1 | 87.4 | 88.8 |
| A49 | 88.9 | 89.1 | 88.8 |
| A50 | 88.9 | 89.1 | 88.8 |
| A51 | 88.1 | 87.4 | 88.8 |
| A52 | 88.5 | 88.2 | 88.8 |
| A53 | 88.9 | 89.1 | 88.8 |
| A54 | 90.71 | 88.24 | 93.46 |
| A55 | 89.82 | 88.24 | 91.59 |
| A56 | 89.38 | 88.24 | 90.65 |
| A57 | 90.27 | 91.6 | 88.79 |
| A58 | 91.59 | 91.6 | 91.59 |
| A59 | 91.15 | 91.6 | 90.65 |
| A60 | 90.71 | 91.6 | 89.72 |
| A61 | 89.82 | 90.76 | 88.79 |
| A62 | 91.15 | 90.76 | 91.59 |
| A63 | 90.71 | 90.76 | 90.65 |
| A64 | 90.27 | 90.76 | 89.72 |
| A65 | 91.15 | 93.28 | 88.79 |
| A66 | 93.36 | 93.28 | 93.46 |
| A67 | 92.48 | 93.28 | 91.59 |
| A68 | 92.04 | 93.28 | 90.65 |
| A69 | 91.59 | 93.28 | 89.72 |
| A70 | 88.5 | 88.24 | 88.79 |
| A71 | 90.71 | 88.24 | 93.46 |
| A72 | 89.38 | 88.24 | 90.65 |
| A73 | 88.94 | 88.24 | 89.72 |
| A74 | 88.94 | 89.08 | 88.79 |
| A75 | 89.82 | 89.08 | 90.65 |
| A76 | 89.38 | 89.08 | 89.72 |
| A77 | 88.5 | 88.24 | 88.79 |
| A78 | 90.71 | 88.24 | 93.46 |
| A79 | 89.38 | 88.24 | 90.65 |
| A80 | 88.94 | 87.39 | 90.65 |
| A81 | 88.5 | 87.39 | 89.72 |
| A82 | 88.05 | 87.39 | 88.79 |
| A83 | 88.94 | 87.39 | 90.65 |
| A84 | 88.5 | 87.39 | 89.72 |
| A85 | 88.1 | 87.4 | 88.8 |
| A86 | 88.5 | 88.24 | 88.79 |
| A87 | 87.2 | 85.7 | 88.8 |
| A88 | 87.3 | 85.7 | 88.8 |
| A89 | 88.1 | 87.4 | 88.8 |
| A90 | 87.2 | 85.7 | 88.8 |
| A91 | 87.3 | 85.7 | 88.8 |
| A92 | 87.7 | 86.6 | 88.8 |
| A93 | 86.9 | 84.9 | 88.8 |
| A94 | 86.9 | 84.9 | 88.8 |
| A95 | 87.7 | 86.6 | 88.8 |
| A96 | 86.9 | 84.9 | 88.8 |
| A97 | 86.9 | 84.9 | 88.8 |
| A98 | 88.5 | 88.2 | 88.8 |
| A99 | 88.1 | 87.4 | 88.8 |
| A100 | 88.1 | 87.4 | 88.8 |
| A101 | 88.5 | 88.2 | 88.8 |
| A102 | 88.9 | 89.1 | 88.8 |
| A103 | 88.9 | 89.1 | 88.8 |
| A104 | 88.9 | 89.1 | 88.8 |

TABLE 11B
Results for binding kinetics determined by SPR
(K_{D} values, kₒₙ and k_{off}) at pH 7.4 and pH 6

| | | kon | | | kon | |
|---|---|---|---|---|---|---|
| | KD [nM] | [1/(M*s)] | koff [1/s] | KD [nM] | [1/(M*s)] | koff [1/s] |
| Antibody | pH 7.4 | pH 7.4 | pH 7.4 | pH 6 | pH 6 | pH 6 |
| A01 | 4.82 | 8.48E+05 | 4.09E−03 | 15.2 | 7.17E+05 | 1.09E−02 |
| A02 | 4.54 | 7.96E+05 | 3.61E−03 | 19.1 | 4.74E+05 | 9.06E−03 |
| A03 | 6.04 | 7.09E+05 | 4.28E−03 | 20.4 | 5.79E+05 | 1.18E−02 |
| A04 | 3.5 | 6.03E+05 | 2.11E−03 | 11.6 | 4.52E+05 | 5.23E−03 |
| A05 | 3.04 | 9.05E+05 | 2.75E−03 | 9.62 | 7.45E+05 | 7.17E−03 |
| A06 | 3.3 | 8.15E+05 | 2.69E−03 | 10.1 | 6.61E+05 | 6.66E−03 |
| A07 | 3.71 | 1.06E+06 | 3.92E−03 | 12.1 | 8.45E+05 | 1.02E−02 |
| A08 | 3.14 | 7.28E+05 | 2.29E−03 | 8.42 | 7.03E+05 | 5.91E−03 |
| A09 | 3.22 | 6.78E+05 | 2.18E−03 | 10.1 | 5.31E+05 | 5.37E−03 |
| A10 | 5.24 | 8.99E+05 | 4.71E−03 | 18.7 | 6.74E+05 | 1.26E−02 |
| A11 | 4.69 | 1.07E+06 | 5.02E−03 | 14.8 | 9.27E+05 | 1.37E−02 |
| A12 | 23 | 1.17E+08 | 2.70E+00 | n.d. | n.d. | n.d. |
| A13 | 20.2 | 3.23E+06 | 6.50E−02 | n.d. | n.d. | n.d. |
| A14 | 19.6 | 7.62E+06 | 1.49E−01 | n.d. | n.d. | n.d. |
| A15 | 2.65 | 1.17E+06 | 3.09E−03 | 9.01 | 9.00E+05 | 8.11E−03 |
| A16 | 15.2 | 6.82E+05 | 1.03E−02 | n.d. | n.d. | n.d. |
| A17 | 17.3 | 5.84E+05 | 1.01E−02 | n.d. | n.d. | n.d. |
| A18 | 17.8 | 7.12E+05 | 1.26E−02 | n.d. | n.d. | n.d. |
| A19 | 13.3 | 7.61E+05 | 1.01E−02 | n.d. | n.d. | n.d. |
| A20 | 14.9 | 6.33E+05 | 9.42E−03 | n.d. | n.d. | n.d. |
| A21 | 20.3 | 4.06E+08 | 8.24E+00 | n.d. | n.d. | n.d. |
| A22 | 13.3 | 6.80E+05 | 9.02E−03 | n.d. | n.d. | n.d. |
| A23 | 20.1 | 8.43E+05 | 1.69E−02 | n.d. | n.d. | n.d. |
| A24 | 13.2 | 6.94E+05 | 9.20E−03 | n.d. | n.d. | n.d. |
| A25 | 14.3 | 6.03E+05 | 8.64E−03 | n.d. | n.d. | n.d. |
| A26 | 14.8 | 5.68E+05 | 8.43E−03 | n.d. | n.d. | n.d. |
| A27 | 13.1 | 7.77E+05 | 1.02E−02 | n.d. | n.d. | n.d. |
| A28 | 4.9 | 4.57E+05 | 2.25E−03 | n.d. | n.d. | n.d. |
| A29 | 8.7 | 3.60E+05 | 3.15E−03 | n.d. | n.d. | n.d. |
| A30 | 3.32 | 7.52E+05 | 2.50E−03 | 8.55 | 7.33E+05 | 6.27E−03 |
| A31 | 26.2 | 7.35E+05 | 1.92E−02 | n.d. | n.d. | n.d. |
| A32 | 4 | 4.25E+05 | 1.70E−03 | n.d. | n.d. | n.d. |
| A33 | 18 | 5.40E+05 | 9.71E−03 | n.d. | n.d. | n.d. |
| A34 | 14.7 | 6.08E+05 | 8.96E−03 | n.d. | n.d. | n.d. |
| A35 | 3.3 | 5.10E+05 | 1.69E−03 | n.d. | n.d. | n.d. |
| A36 | 4.5 | 4.37E+05 | 1.95E−03 | n.d. | n.d. | n.d. |
| A37 | 4.2 | 4.30E+05 | 1.82E−03 | n.d. | n.d. | n.d. |
| A38 | 5.6 | 4.77E+05 | 2.67E−03 | n.d. | n.d. | n.d. |
| A39 | 6.9 | 4.39E+05 | 3.02E−03 | n.d. | n.d. | n.d. |
| A40 | 4.4 | 4.67E+05 | 2.07E−03 | n.d. | n.d. | n.d. |
| A41 | 4.6 | 4.34E+05 | 1.98E−03 | n.d. | n.d. | n.d. |
| A42 | 3.8 | 5.97E+05 | 2.26E−03 | n.d. | n.d. | n.d. |
| A43 | 4.2 | 4.68E+05 | 1.96E−03 | n.d. | n.d. | n.d. |
| A44 | 4.7 | 3.93E+05 | 1.83E−03 | n.d. | n.d. | n.d. |
| A45 | 3.1 | 4.65E+05 | 1.44E−03 | n.d. | n.d. | n.d. |
| A46 | 5.3 | 4.32E+05 | 2.28E−03 | n.d. | n.d. | n.d. |
| A47 | 5.2 | 4.19E+05 | 2.18E−03 | n.d. | n.d. | n.d. |
| A48 | 4.9 | 5.69E+05 | 2.77E−03 | n.d. | n.d. | n.d. |
| A49 | 5.4 | 4.49E+05 | 2.41E−03 | n.d. | n.d. | n.d. |
| A50 | 40.7 | 2.25E+09 | 9.16E+01 | n.d. | n.d. | n.d. |
| A51 | 1.9 | 4.53E+05 | 8.44E−04 | n.d. | n.d. | n.d. |
| A52 | 4 | 5.36E+05 | 2.12E−03 | n.d. | n.d. | n.d. |
| A53 | 4.2 | 4.77E+05 | 2.01E−03 | n.d. | n.d. | n.d. |
| A54 | 3.24 | 9.11E+05 | 2.95E−03 | 11.6 | 6.74E+05 | 7.84E−03 |
| A55 | 2.65 | 1.08E+06 | 2.86E−03 | 8.31 | 9.03E+05 | 7.50E−03 |
| A56 | 2.61 | 1.06E+06 | 2.78E−03 | 7.75 | 9.47E+05 | 7.34E−03 |
| A57 | 4.78 | 1.03E+06 | 4.94E−03 | 19.3 | 6.66E+05 | 1.29E−02 |
| A58 | 6.57 | 6.47E+05 | 4.25E−03 | 24.6 | 4.62E+05 | 1.13E−02 |
| A59 | 5.12 | 7.95E+05 | 4.07E−03 | 22.2 | 4.70E+05 | 1.05E−02 |
| A60 | 5.27 | 8.56E+05 | 4.51E−03 | 19.3 | 5.97E+05 | 1.15E−02 |
| A61 | 4.78 | 9.57E+05 | 4.57E−03 | 16.2 | 7.35E+05 | 1.19E−02 |
| A62 | 4.98 | 8.26E+05 | 4.12E−03 | 17.4 | 6.04E+05 | 1.05E−02 |
| A63 | 4.84 | 8.16E+05 | 3.95E−03 | 18.8 | 5.36E+05 | 1.01E−02 |
| A64 | 6.27 | 6.73E+05 | 4.22E−03 | 21.2 | 4.94E+05 | 1.05E−02 |
| A65 | 10.3 | 6.54E+05 | 6.76E−03 | 36.3 | 4.86E+05 | 1.76E−02 |
| A66 | 8.07 | 8.10E+05 | 6.54E−03 | 34.7 | 4.71E+05 | 1.64E−02 |
| A67 | 8.05 | 7.68E+05 | 6.18E−03 | 31.6 | 5.00E+05 | 1.58E−02 |
| A68 | 6.97 | 8.83E+05 | 6.15E−03 | 23.4 | 6.72E+05 | 1.58E−02 |
| A69 | 7.19 | 9.44E+05 | 6.79E−03 | 23.1 | 7.48E+05 | 1.73E−02 |
| A70 | 2.78 | 8.86E+05 | 2.46E−03 | 9.47 | 6.44E+05 | 6.10E−03 |
| A71 | 2.76 | 7.97E+05 | 2.20E−03 | 10 | 5.46E+05 | 5.46E−03 |
| A72 | 3.55 | 5.83E+05 | 2.07E−03 | 13.1 | 3.88E+05 | 5.07E−03 |
| A73 | 3.05 | 7.61E+05 | 2.32E−03 | 12.9 | 4.28E+05 | 5.54E−03 |
| A74 | 3.83 | 7.74E+05 | 2.97E−03 | 13.9 | 5.25E+05 | 7.30E−03 |
| A75 | 3.21 | 8.20E+05 | 2.64E−03 | 10.7 | 6.12E+05 | 6.54E−03 |
| A76 | 3.25 | 8.60E+05 | 2.80E−03 | 12.4 | 5.54E+05 | 6.89E−03 |
| A77 | 5.21 | 7.49E+05 | 3.90E−03 | 17.3 | 5.79E+05 | 1.00E−02 |
| A78 | 5.25 | 6.77E+05 | 3.55E−03 | 19 | 4.85E+05 | 9.19E−03 |
| A79 | 4.39 | 8.14E+05 | 3.57E−03 | 15.6 | 5.77E+05 | 9.03E−03 |
| A80 | 4.1 | 5.28E+05 | 2.16E−03 | 11.5 | 4.56E+05 | 5.23E−03 |
| A81 | 4.37 | 5.38E+05 | 2.35E−03 | 13.6 | 4.11E+05 | 5.59E−03 |
| A82 | 5.78 | 8.99E+05 | 5.19E−03 | 22.9 | 5.82E+05 | 1.34E−02 |
| A83 | 4.75 | 9.98E+05 | 4.75E−03 | 13.9 | 9.20E+05 | 1.28E−02 |
| A84 | 4.85 | 1.09E+06 | 5.27E−03 | 16.4 | 8.60E+05 | 1.41E−02 |
| A85 | 7.2 | 1.55E+06 | 1.11E−02 | n.d. | n.d. | n.d. |
| A86 | 3.27 | 9.67E+05 | 3.16E−03 | 13.7 | 5.74E+05 | 7.88E−03 |
| A87 | 8.7 | 1.81E+06 | 1.58E−02 | n.d. | n.d. | n.d. |
| A88 | 11.8 | 1.45E+06 | 1.71E−02 | n.d. | n.d. | n.d. |
| A89 | 77.5 | n.a. | n.a. | n.d. | n.d. | n.d. |
| A90 | 9.7 | 1.78E+06 | 1.73E−02 | n.d. | n.d. | n.d. |
| A91 | 13.7 | 1.07E+06 | 1.46E−02 | n.d. | n.d. | n.d. |
| A92 | 74.3 | n.a. | n.a. | n.d. | n.d. | n.d. |
| A93 | 91.7 | n.a. | n.a. | n.d. | n.d. | n.d. |
| A94 | 110.0 | n.a. | n.a. | n.d. | n.d. | n.d. |
| A95 | n.d. | n.d. | n.d. | n.d. | n.d. | n.d. |
| A96 | 90.3 | n.a. | n.a. | n.d. | n.d. | n.d. |
| A97 | 108.0 | n.a. | n.a. | n.d. | n.d. | n.d. |

| | | | | |
|---|---|---|---|---|
| A98 | no binding at 50 nM | n.d. | n.d. | n.d. |
| A99 | no binding at 50 nM | n.d. | n.d. | n.d. |
| A100 | no binding at 50 nM | n.d. | n.d. | n.d. |
| A101 | no binding at 50 nM | n.d. | n.d. | n.d. |
| A102 | >50 nM | n.d. | n.d. | n.d. |
| A103 | >50 nM | n.d. | n.d. | n.d. |
| A104 | >50 nM | n.d. | n.d. | n.d. |

n.d. = not determined
n.a. = not applicable (fit done in steady state)

The SPR data show that most of the exemplary antibodies bind to the ECD of LAYN isoform 2 with high affinity (K_{D} value less than 50 nM). However, no specific binding at 50 nM was detected for antibodies A98-A101. Antibodies A89, A92-A94, A96, A97 and A102-A104 bound to LAYN with a K_{D} value of more than 50 nM. In addition, binding of exemplary antibodies to the ECD of LAYN isoform 2 could even be demonstrated at pH 6.

Next, Response Level Correlation Assay (RLCA) by SPR was performed on stressed samples of exemplary antibodies A1 and A2 to determine if binding is maintained in stressed samples. Briefly, a Biacore 8K (Cytiva) and the ‘Serial Concentration using Capture’ method in the Biacore 8K Control Software (version 5.0.18.22405) were used. Protein A/G was amine coupled to the surface of a CM5 chip to a surface density of 2000-2500 RU using a standard amine coupling kit from the manufacturer. The running buffer was HBS-EP+ and the flow rate was 10 μl/min. Serial dilutions, 2:3, (10 μg/ml-0.9 μg/ml) of the unstressed antibody (TO) were captured on the protein A/G for 60 seconds. LAYN-v2-His (SEQ ID NO: 190) was injected at 15 μg/ml (~600 nM) over the surface for 400 seconds (association). Stressed samples at 2 μg/ml were captured for 60 seconds followed by injection of 15 μg/ml LAYN-v2-His for 400 seconds.

Different stress conditions were investigated. For thermal stress condition the antibody was incubated in an acidic formulation buffer pH 6 at 40 °C for 4 weeks using a high antibody concentration of 150 g/L and a low antibody concentration of 10 g/L. To investigate physiological stress conditions, the antibody was incubated at 10 g/L in PBS pH 7.4 at 37 °C for 4 weeks.

Biacore Insight Evaluation software (version 5.0.18.22102) was used to create calibration curves from the TO capture and late binding data. The capture and late binding response levels of the stressed samples were used in conjunction with the calibration curves to calculate the active concentrations. The late binding active concentration was divided by the capture active concentration and expressed as a percentage. For the reported values and graphs, the percent active concentration was normalized against TO values.

Results are shown in Table 12.

TABLE 12
Relative active concentration characterization of thermal and high
concentration stressed samples to LAYN-v2-His
compared to unstressed antibody A1 and A2 by RLCA.

| | Normalized % Active Concentration Relative to Control | |
|---|---|---|
| Condition | Antibody A1 | Antibody A2 |
| Thermal Stress | 101% | 100 % |
| (10 g/L, pH 6, 40° C., 4 W) | | |
| High Concentration & | 101% | 100 % |
| Thermal Stress | | |
| (150 g/L, pH 6, 40° C., 4 W) | | |
| Physiological Stress | 101% | 101% |
| (10 g/L, PBS, pH 7.4, 37° C., | | |
| 4 W) | | |

These data show that all stressed samples maintained active binding compared to the unstressed sample despite thermal stress (40 °C) and/or high concentration (150 g/L).

### Example 4: Characterization of Anti-LAYN Antibodies: Purity and Thermal Stability

The antibodies were further characterized with regard to purity/aggregates and thermal stability.

To determine purity/aggregates, analytical size exclusion chromatography (aSEC) was used. Briefly, the size heterogeneity of protein species in samples of the antibodies were quantitated by molecular sieving through a stationary phase composed of porous polymeric beads. The characterization was performed with an Acquity UPLC BEH200SEC column installed on an Agilent 1260 HPLC system equipped with a UV detector. Samples (30 μg) were injected onto the column. Chromatograms were obtained at wavelengths of 214 nm and 280 nm. Two different columns and methods were used, for details see Table 13. The percentage and retention time range of the size species (high molecular weight (HMW), main, low molecular weight (LMW)), was quantitated by the ApexTrack integration algorithm of the chromatograms collected at 280 nm with Empower 3 Pro analysis software (Waters Corp.). Results from both methods are comparable and are indicated in Table 14, respectively.

TABLE 13
Parameters aSEC Method

| Method | A | B |
|---|---|---|
| System | Agilent 1260 HPLC | Agilent 1260 HPLC |
| Column | Acquity UPLC BEH200SEC, | Acquity UPLC BEH200SEC, |
| | 1.7 μm particle, 200 Å, | 1.7 μm particle, 200 Å, |
| | 4.6 × 300 mm | 4.6 × 150 mm |
| Buffers | 40 mM sodium phosphate, | 50 mM Na Phosphate, 200 |
| | 400 mM sodium perchlorate, | mM Arginine-HCL, 0.05% |
| | pH 6.8 | Na Azide, pH 6.8 |
| Gradient | Isocratic | Isocratic |
| Run Time | 16 min | 5 min |
| Target load | 30 μg | 30 μg |
| Flowrate | 0.3 mL/min | 0.5 mL/min |
| Column | 25° C. | 37° C. |
| temperature | | |
| Sample | 5° C. | 5° C. |
| temperature | | |
| Detection | 214 nm and 280 nm, | 214 nm and 280 nm, |
| | Bandwith 4 nm | Bandwith 4 nm |

Results are shown in Table 14 below as “% monomer” determined as % area (UV-signal) of monomer peak for the various antibodies.

Thermal stability was analyzed by thermal unfolding using (dye-based) Differential Scanning Fluorimetry (DSF). For DSF measurements, SYPRO® Orange dye (5000× stock; Invitrogen, Inc., Carlsbad, CA) was added at a 5× final dye concentration to the antibodies at 0.4 mg/ml concentration in 10 mM histidine-HCl (pH 6). Samples were transferred to polypropylene 96-well PCR plates in addition to 5 microliters of diluted dye. After an initial 20 °C equilibration for 10 min, using a QuantStudio5 RT-PCR machine, a temperature ramp from 25 °C to 90 °C in 1 °C per minute increments with a 10 s equilibration step prior to taking fluorescence measurements was applied. The temperature of hydrophobic exposure (Th) was calculated by the software using a mathematical second-derivative method (d (RFU)/dT) to calculate the inflection point of the curve.

Thermal unfolding of the Fab is summarized as “Fab Tm” in Table 14 below for various antibodies. It is noted that the constant domains of antibodies A1-A104 are identical.

TABLE 14

| aSEC (% monomer, method B) and DSF (Fab Tm) | | |
|---|---|---|
| results for antibodies A1 - A104. | | |
| Antibody | % monomer | Fab Tm [° C.] |
| A01 | 99.8 | 88.2 |
| A02 | 98.9 | 88.0 |
| A03 | 99.7 | 88.4 |
| A04 | 98.8 | 88.3 |
| A05 | 98.9 | 87.3 |
| A06 | 99.0 | 87.4 |
| A07 | 98.9 | 88.5 |
| A08 | 98.9 | 86.7 |
| A09 | 98.9 | 86.8 |
| A10 | 98.7 | 88.7 |
| A11 | 98.9 | 89.0 |
| A12 | 99.4 | 89.3 |
| A13 | 99.3 | 88.6 |
| A14 | 99.4 | 88.5 |
| A15 | 99.3 | 89.7 |
| A16 | 99.4 | 88.5 |
| A17 | 99.4 | 88.5 |
| A18 | 98.9 | n.d. |
| A19 | 99.3 | 89.2 |
| A20 | 99.3 | 89.1 |
| A21 | 99.1 | 88.8 |
| A22 | 98.9 | 88.6 |
| A23 | 99.3 | 87.7 |
| A24 | 99.4 | 88.7 |
| A25 | 99.3 | 89.4 |
| A26 | 99.3 | 88.5 |
| A27 | 99.2 | 89.1 |
| A28 | 99.3 | 86.2 |
| A29 | 99.3 | 84.8 |
| A30 | 99.2 | 87.0 |
| A31 | 99.2 | 88.0 |
| A32 | 99.2 | 88.8 |
| A33 | 99.3 | 89.0 |
| A34 | 99.1 | n.d. |
| A35 | 99.1 | 90.1 |
| A36 | 99.2 | 89.4 |
| A37 | 99.2 | 89.4 |
| A38 | 99.1 | 87.9 |
| A39 | 99.1 | 89.2 |
| A40 | 99.1 | 88.7 |
| A41 | 99.0 | 88.9 |
| A42 | 98.7 | 89.7 |
| A43 | 98.9 | 89.1 |
| A44 | 99.1 | n.d. |
| A45 | 99.1 | 89.0 |
| A46 | 99.0 | 88.4 |
| A47 | 99.0 | 89.8 |
| A48 | 98.8 | 89.4 |
| A49 | 98.8 | 87.9 |
| A50 | 98.9 | 89.3 |
| A51 | 98.6 | 87.9 |
| A52 | 98.6 | 88.4 |
| A53 | 98.1 | 89.5 |
| A54 | 99.4 | 88.5 |
| A55 | 99.5 | 89.0 |
| A56 | 99.8 | 89.1 |
| A57 | 94.7 | 88.7 |
| A58 | 99.7 | 88.7 |
| A59 | 99.9 | 89.1 |
| A60 | 99.6 | 89.2 |
| A61 | 97.3 | 88.5 |
| A62 | 99.8 | 88.3 |
| A63 | 99.8 | 88.5 |
| A64 | 99.8 | 88.8 |
| A65 | 94.8 | 88.5 |
| A66 | 99.8 | 88.3 |
| A67 | 99.8 | 88.5 |
| A68 | 99.8 | 88.6 |
| A69 | 99.7 | 88.8 |
| A70 | 97.0 | 88.5 |
| A71 | 77.8 | 88.2 |
| A72 | 98.7 | 88.7 |
| A73 | 99.1 | 88.9 |
| A74 | 96.2 | 87.8 |
| A75 | 99.0 | 87.8 |
| A76 | 99.7 | 88.1 |
| A77 | 96.8 | 88.2 |
| A78 | 98.9 | 87.7 |
| A79 | 98.9 | 88.3 |
| A80 | 98.7 | 87.1 |
| A81 | 99.2 | 87.6 |
| A82 | 96.9 | 89.0 |
| A83 | 98.5 | 89.0 |
| A84 | 98.6 | 89.3 |
| A85 | 96.9 | 89.4 |
| A86 | 97.2 | 89.4 |
| A87 | 97.9 | 89.7 |
| A88 | n.d. | n.d. |
| A89 | 97.8 | 89.7 |
| A90 | 97.3 | 90.0 |
| A91 | n.d. | n.d. |
| A92 | n.d. | n.d. |
| A93 | n.d. | n.d. |
| A94 | n.d. | n.d. |
| A95 | n.d. | n.d. |
| A96 | n.d. | n.d. |
| A97 | n.d. | n.d. |
| A98 | 99.4 | 87.8 |
| A99 | 99.3 | 87.3 |
| A100 | 99.2 | 89.6 |
| A101 | 99.0 | 86.7 |
| A102 | 98.9 | n.d. |
| A103 | 99.0 | 87.9 |
| A104 | 98.8 | 86.4 |

These data show a high thermal stability of the Fab portion in the exemplary antibodies of more than 84 °C with only one antibody (A29) having a Fab Tm below 86 °C and most antibodies even having a Fab Tm of more than 88 °C. For comparison, a standard set of antibodies described in the literature shows Fab Tm values in the range from 57 to 82 °C with an average of 76 °C (Garber E, Demarest S J. A broad range of Fab stabilities within a host of therapeutic IgGs. Biochem Biophys Res Commun. 2007 Apr. 13; 355(3):751-7. doi: 10.1016/j.bbrc.2007.02.042. Epub 2007 Feb. 16). As described in this reference, stability is a requirement for the development of antibodies for therapeutic or diagnostic applications.

In addition, aSEC analysis was performed for antibodies A1 and A2. Antibodies were stored up to 4 weeks under physiological conditions at 37 °C and PBS buffer pH 7.4 at a low concentration of 10 g/L, to assess stability and potential degradation of the antibodies. Results are shown in Table 15 below.

TABLE 15
High molecular weight species (HMWS), main peak, and low
molecular weight species (LMWS) data from aSEC
characterization (method A) of antibodies A1 and A2
in PBS at 37° C. at different time points.

| | | Time | | % | |
|---|---|---|---|---|---|
| | | Point | % | Main | % |
| Antibody | Condition | (Week) | HMWS | Peak | LMWS |
| A1 | PBS, 10 g/L, 37° C. | 0 | 2.0 | 96.9 | 1.1 |
| | | 2 | 2.7 | 95.4 | 2.0 |
| | | 4 | 2.8 | 94.8 | 2.5 |
| A2 | PBS, 10 g/L, 37° C. | 4 | 1.8 | 95.8 | 2.3 |

These data show acceptable stability for storage
at 37° C. for up to 4 weeks for both
antibodies (under physiological conditions).

### Example 5: Binding of Anti-LAYN Antibodies to Activated Tregs

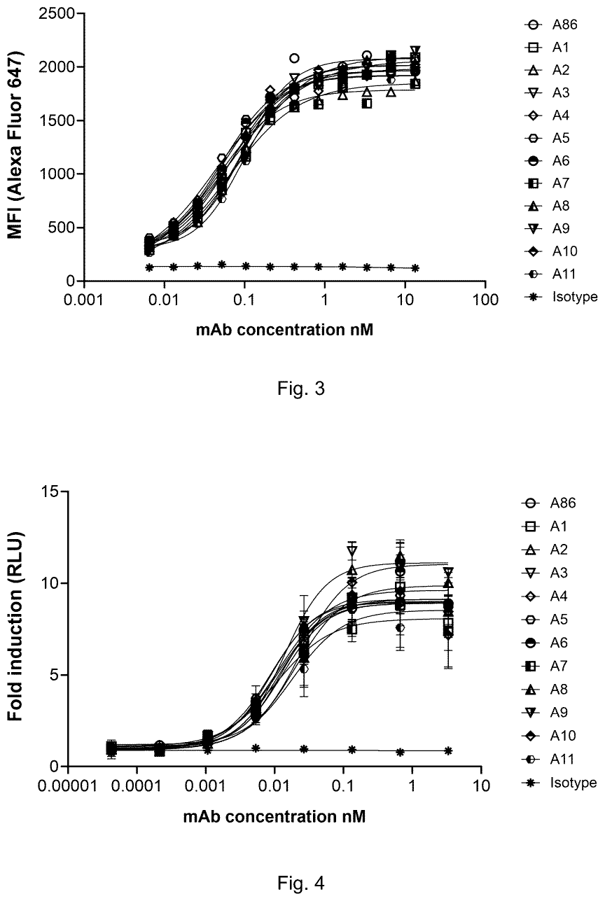

To determine the binding of anti-LAYN antibodies to activated regulatory T cells (Tregs), anti-LAYN antibodies were incubated with activated human Tregs prepared from previously isolated and expanded Tregs that were stimulated for 7 days with anti-CD3/CD28 beads (Dynabeads, 1:3 bead to cell ratio) in the presence of 200 units/mL of recombinant human IL-2. 75% of the activated Tregs were LAYN positive. The antibodies were serially diluted from a starting concentration of 2 μg/mL. An appropriate Alexa Fluor 647-conjugated secondary antibody (Thermo Fisher Scientific) against the primary anti-LAYN antibody was applied, incubated for 30 min at 4 °C, and then washed off. Additionally LIVE/DEAD™ Fixable Aqua Dead Cell Stain was added to discriminate viable cells. Samples were processed on a flow cytometer and data was analyzed using FlowJo (FlowJo Software, Ashland, OR). EC50 values for the binding of selected anti-LAYN antibodies to activated Tregs was calculated using GraphPad Prism (GraphPad Software, La Jolla, CA). All antibodies used in this example were afucosylated. Results of a representative experiment are shown in FIG. 3 and Table 16 below. Mean average EC50 values determined from three independent experiments are summarized in Table 17 below.

TABLE 16

| Affinity (EC50) of anti-LAYN antibodies | |
|---|---|
| binding to activated Tregs. | |
| Anti-LAYN mAb | EC₅₀ (nM) |
| A1 | 0.061 |
| A2 | 0.047 |
| A3 | 0.066 |
| A4 | 0.052 |
| A5 | 0.045 |
| A6 | 0.052 |
| A7 | 0.066 |
| A8 | 0.085 |
| A9 | 0.091 |
| A10 | 0.069 |
| A11 | 0.094 |
| A86 | 0.097 |

TABLE 17
Affinity (EC₅₀) of anti-LAYN antibodies binding to activated Tregs
(mean and standard deviation (SD) of 3 independent experiments)

| Anti-LAYN mAb | Mean EC₅₀ (nM) | SD (nM) |
|---|---|---|
| A1 | 0.076 | 0.011 |
| A2 | 0.080 | 0.027 |
| A3 | 0.077 | 0.010 |
| A4 | 0.086 | 0.028 |
| A5 | 0.071 | 0.023 |
| A6 | 0.088 | 0.029 |
| A7 | 0.079 | 0.010 |
| A8 | 0.100 | 0.023 |
| A9 | 0.127 | 0.057 |
| A10 | 0.093 | 0.030 |
| A11 | 0.141 | 0.062 |
| A86 | 0.096 | 0.005 |

These data show that all tested anti-LAYN antibodies specifically bind to activated Tregs expressing LAYN.

### Example 6: ADCC and ADCP Reporter Assay Activity of Anti-LAYN Antibodies

Without being bound to any theory, the present inventors assume that the anti-LAYN antibodies described herein deplete intratumoral regulatory T cells (Tregs) in that (i) the antibodies bind to LAYN expressed by said Tregs, and (ii) kill the Tregs by means of antibody dependent cell cytotoxicity (ADCC) or antibody dependent cell phagocytosis (ADCP).

Natural killer cells mediate ADCC mainly via Fc gamma (Fcg) receptor IIIA (CD16A). Therefore, the ADCC potential of anti-LAYN antibodies was measured by the capacity of these antibodies to mediate crosslinking of CD16AV158 expressed by Nuclear Factor of Activated T-cells (NFAT) luciferase reporter cells. LAYN-expressing Expi293 cells were co-cultured in a 1:5 ratio with CD16V158-expressing Jurkat NFAT luciferase reporter cells at 37 °C for 4 h in the presence of an anti-LAYN antibody or a control antibody (isotype control). BIO-GLO™ (Promega, Madison, WI) was added to the cells and then the luminescence was read for measurement of luciferase activity. The half-maximal effective concentration (EC50) was calculated using GraphPad Prism. All antibodies used in this example were afucosylated. Results of a representative experiment are shown in FIG. 4 and Table 18 below. Mean average EC50 values determined from three independent experiments are summarized in Table 19 below.

TABLE 18
Cross-linking of CD16A expression reporter cells
by anti-LAYN antibodies

| Anti-LAYN mAb | EC₅₀ (nM) |
|---|---|
| A1 | 0.012 |
| A2 | 0.011 |
| A3 | 0.014 |
| A4 | 0.009 |
| A5 | 0.009 |
| A6 | 0.011 |
| A7 | 0.011 |
| A8 | 0.023 |
| A9 | 0.020 |
| A10 | 0.013 |
| A11 | 0.018 |
| A86 | 0.011 |

TABLE 19
Cross-linking of CD16A expression reporter cells by anti-LAYN
antibodies (mean EC₅₀ and standard deviation (SD) of
independent experiments)

| Anti-LAYN mAb | Mean EC₅₀ (nM) | SD (nM) |
|---|---|---|
| A1 | 0.014 | 0.003 |
| A2 | 0.012 | 0.006 |
| A3 | 0.019 | 0.009 |
| A4 | 0.016 | 0.009 |
| A5 | 0.010 | 0.002 |
| A6 | 0.014 | 0.007 |
| A7 | 0.013 | 0.008 |
| A8 | 0.021 | 0.009 |
| A9 | 0.024 | 0.003 |
| A10 | 0.016 | 0.004 |
| A11 | 0.023 | 0.005 |
| A86 | 0.014 | 0.003 |

These data demonstrate that all tested antibodies showed a comparable capacity for CD16A engagement and signaling activation upon binding to LAYN-expressing Jurkat cells, which is an essential prerequisite for natural killer cell-mediated ADCC.

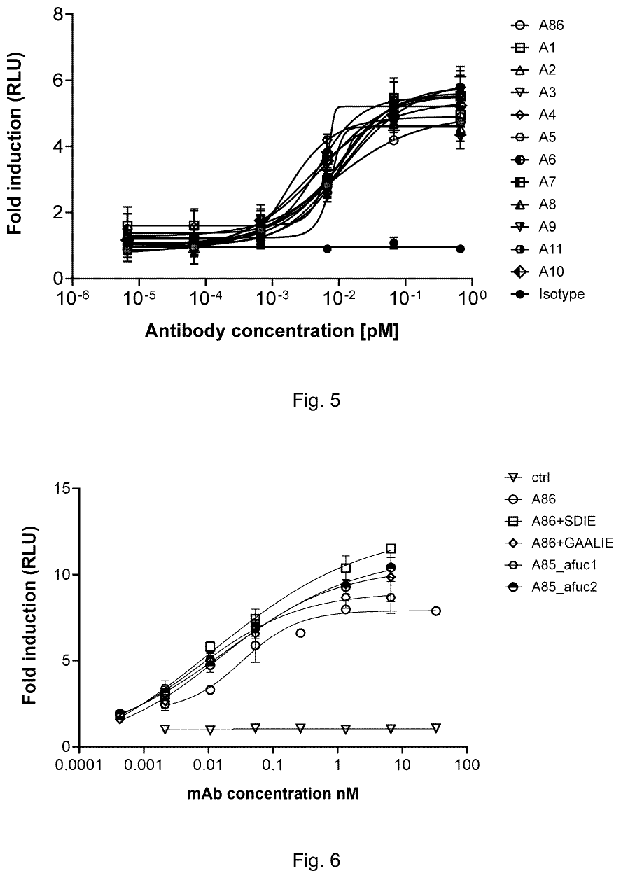

ADCP is mediated by monocytes and macrophages expressing a plethora of stimulatory Fcg receptors like FcgRIIA and FcgRIIIA. The ADCP potential of anti-LAYN antibodies was measured by the capacity of these antibodies to activate monocytic THP-1 reporter cells expressing a luciferase reporter under a minimal Nuclear Factor kappa-light-chain-enhancer of activated B cells (NFkB) promoter. LAYN-expressing Jurkat cells were co-cultured in a 1:1 ratio with THP-1 luciferase reporter cells at 37 °C for 4 h in the presence of an anti-LAYN antibody or a control antibody (isotype control). Bio-Glo-NL™ (Promega, Madison, WI) was added to the cells and then the luminescence was read for measurement of luciferase activity. The half-maximal effective concentration (EC50) was calculated using GraphPad Prism. All antibodies used in this example were afucosylated. Results are shown in FIG. 5 and Table 20 below.

TABLE 20
ADCP reporter activity of anti-LAYN antibodies

| Anti-LAYN mAb | EC₅₀ (nM) |
|---|---|
| A1 | 0.006 |
| A2 | 0.011 |
| A3 | 0.004 |
| A4 | 0.010 |
| A5 | 0.002 |
| A6 | 0.005 |
| A7 | 0.009 |
| A8 | 0.007 |
| A9 | 0.007 |
| A10 | 0.004 |
| A11 | 0.014 |
| A86 | 0.007 |

These data demonstrate that all tested antibodies showed a comparable capacity for THP-1 activation upon binding to LAYN-expressing Jurkat cells which is a direct measure for macrophage-mediated ADCP.

### Example 7: ADCC of Anti-LAYN Antibodies

The capacity of anti-LAYN antibodies to induce ADCC-mediated killing of LAYN-expressing cells was evaluated in an impedance-based cell viability assay. CD16-KILR cells (Eurofins Discovery) were used as CD16AV158-expressing effector cells and cultured according to manufacturer's instructions prior to the experiment. In this assay, LAYN-expressing HEK293 cells were seeded in a 384-well CytoView-Z plate (Axion Biosystems) to establish a monolayer. Cell growth and viability was monitored by impedance measurement with the Maestro ZHT (Axion Biosystems). After reaching an impedance equivalent to 50% confluency, CD16-KILR cells were co-cultured with LAYN-expressing HEK cells at a ratio of 1:1 in the presence of titrations of anti-LAYN antibodies or an anti-LAYN antibody (A86) having a mutated IgG1 Fc that abolishes FcγR binding as negative control for up to 24 hours at 37 °C and 5% CO₂. Maximum lysis of LAYN-expressing HEK293 cells was assessed by addition of 0.0025% Tween. The negative control was used as no lysis reference. Impedance was measured over the whole assay period. The area under the curve (AUC) of the impedance (normalized to assays start) is used to calculate % killing. % Killing=(1−(specific lysis−maximum lysis)/(no lysis−maximum lysis))×100. Results are shown in Table 21.

TABLE 21
ADCC of LAYN-expressing HEK cells by anti-LAYN antibodies

| Anti-LAYN mAb | EC₅₀ (pM) |
|---|---|
| A1 | 3.10 |
| A2 | 1.03 |
| A3 | 4.23 |
| A4 | 0.12 |
| A5 | 0.34 |
| A6 | 2.08 |
| A7 | 9.61 |
| A8 | 7.67 |
| A9 | 5.90 |
| A10 | 2.04 |
| A11 | 0.91 |

These data demonstrate that anti-LAYN antibodies mediate cytotoxicity of LAYN-positive HEK cells resulting in 90-100% maximum killing of target cells for all tested antibodies.

### Example 8: Design and Characterization of Anti-LAYN Fc Variant Antibodies

To increase ADCC potency different anti-LAYN antibodies with different Fc versions, as shown in Table 22 below, were designed, produced and characterized.

TABLE 22
Fc variant antibodies

| | Heavy chain SEQ ID NOs | | | | | Light chain SEQ ID NOs | | | | |
|---|---|---|---|---|---|---|---|---|---|---|
| mAb | CDR1 | CDR2 | CDR3 | VH | HC | CDR1 | CDR2 | CDR3 | VL | LC |
| A86 + GAALIE | 1 | 19 | 32 | 34 | 171 | 24 | 5 | 33 | 79 | 81 |
| A86 + SDIE | 1 | 19 | 32 | 34 | 172 | 24 | 5 | 33 | 79 | 81 |
| A85_afuc1 | 1 | 19 | 142 | 143 | 144 | 24 | 5 | 6 | 79 | 81 |
| A85_afuc2 | 1 | 19 | 142 | 143 | 144 | 24 | 5 | 6 | 79 | 81 |

Briefly, antibodies A86+GAALIE and A86+SDIE differ from antibody A86 described above only in that they include either a G236A/A330L/1332E (“GAALIE”) or a S239D/1332E (“SDIE”) mutation in the heavy chain Fc region (according to the EU-index of Kabat). Accordingly, antibodies A86+GAALIE and A86+SDIE have the same VH and VL sequences as antibody A86.

Antibodies A85_afuc1 and A85_afuc2 are afucosylated versions of antibody A85 obtained by different methods/cell lines to produce/express afucosylated antibodies. Accordingly, the amino acid sequences of antibodies A85_afuc1 and A85_afuc2 correspond to those of antibody A85, while the glycosylation of the antibodies is altered. Antibody A85_afuc1 was produced essentially as described in WO 2011/035884 A1.

Antibody A85_afuc2 was produced in a CHO Fut8 KO cell line, which lacks fucosyltransferase 8 (Fut8). Fut8 is the enzyme responsible for transferring fucose on the glycosylation tree in the golgi. Hence the knockout (KO) of this enzyme leads to afucosylated proteins and antibodies produced with this cell line. The CHO-K1 FUT8/host cell line (also referred to as “CHO Fut8 KO” cell line) was produced from a proprietary CHO-K1 cell line essentially as described in Malphettes et al., 2010 (Malphettes L, Freyvert Y, Chang J, Liu P Q, Chan E, Miller J C, Zhou Z, Nguyen T, Tsai C, Snowden A W, Collingwood T N, Gregory P D, Cost G J. Highly efficient deletion of FUT8 in CHO cell lines using zinc-finger nucleases yields cells that produce completely nonfucosylated antibodies. Biotechnol Bioeng. 2010 Aug. 1; 106(5):774-83. doi: 10.1002/bit.22751) using CompoZr® Custom Zinc Finger Nucleases (Sigma-Aldrich, CSTZFN-1KT, Lot No. 12030921MN). Eleven days after zinc-finger nuclease (ZFN) transfection, the transfected CHO cells were single cell cloned into 384-well plates using fluorescent activated cell sorting on a FACSMelody instrument (BD Biosciences). Afterwards, monoclonal cell populations were picked and expanded followed by genetic as well as functional characterization of the established monoclonal host cell line candidates. Thereby, the CHO-K1 FUT8^{-/-} host cell line (clone E002) was generated.

Antibodies A86+GAALIE, A86+SDIE, A85_afuc1 and A85_afuc2 were characterized regarding % monomer, Tm Fab and Tm Fc as described in Example 4 above (for the panel of antibodies including antibodies A85 and A86). Results are shown in Table 23 below:

TABLE 23
Characterization of anti-LAYN Fc variant antibodies

| | % | | |
|---|---|---|---|
| Antibody | monomer | Tm Fab [°C] | Tm Fc [°C] |
| A85_afuc1 | 96.92 | 88.3 | 71.7 |
| A85_afuc2 | 96.92 | 88.2 | 71.5 |
| A86 + GAALIE | 99.7 | 89.5 | 57.9 |
| A86 + SDIE | 99.7 | 88.7 | 48.8 |

These data show that the characteristics “% monomer” and “Tm Fab” of the Fc variant antibodies essentially correspond to the respective wt-Fc antibodies described above. Accordingly, no relevant influence of the Fc variations on these characteristics was observed. However, with regard to Tm Fc, considerably lower Tm Fc were observed for G236A/A330L/1332E (“GAALIE”) and S239D/1332E (“SDIE”) Fc.

### Example 9: CD16 Binding of Anti-LAYN Fc Variant Antibodies

The affinities of anti-LAYN Fc variant antibodies described in Example 8 above to human Fc gamma Receptor Illa (CD16a) was determined via SPR on a Biacore 8K+ instrument using HEPES buffered saline with EDTA (HBS-EP+) containing 1 M HEPES (2-(4-(2-Hydroxyethyl)-1-piperazinyl)-ethansulfonic acid), 1.5 M NaCl, 0.03 M EDTA and 0.5% v/v polysorbate 20 (Cytiva #BR-100668) as running buffer. Following manufacturer's recommendations, a CM5 biosensor chip (Cytiva #BR100530) was activated with a 50:50 solution of 1-Ethyl-3-(3-dimethylaminopropyl) carbodiimide/N-Hydroxysuccinimide (EDC/NHS) (amine coupling kit Cytiva #BR-100633). Protein A/G at 10 μg/mL in 10 mM acetate pH 4.5 was immobilized on the surface followed by deactivation with 1 M ethanolamine HCl.

Anti-LAYN antibodies were captured at 2 μg/mL in HBS-EP+ for 30 seconds at a flowrate of 10 μL/min. Ten serial dilutions (2-fold) of human Fc gamma receptors were prepared in HBS-EP+ staring with 10 μM and injected for 120 seconds association at 30 μL/min followed by a dissociation of 120 seconds. The surface was regenerated by injecting 0.85% phosphoric acid for 30 sec at a flowrate of 30 μL/min.

The analyte interaction with sensor surface (flow cell 1) and blank (HBS-EP⁺ or 0 nM analyte) were subtracted from the raw data. K_{D} values were calculated using Steady state binding analysis for both CD16a low and high affinity versions of CD16a: human FcγRIIIa-F158 (CD16a) and human FcγRIIIa-V158 (CD16a). Results are shown in Table 24.

TABLE 24
CD16 binding of anti-LAYN antibodies

| | CD16A F158 | CD16A V158 |
|---|---|---|
| Anti-LAYN mAb | KD [μM] | KD [μM] |
| A86 | 0.28 | 0.31 |
| A86 + GAALIE | 0.030 | 0.029 |
| A86 + SDIE | 0.006 | 0.006 |
| A85_afuc1 | 0.041 | 0.040 |

The data show that all three Fc variants including afucosylation increase binding to Fc gamma receptor CD16a high and low affinity version by a factor about 10 as compared to the anti-LAYN antibody with wildtype Fc.

### Example 10: ADCC Reporter Assay Activity

The ADCC potential of the above described anti-LAYN Fc variant antibodies was measured by the capacity of these antibodies to mediate crosslinking of CD16AV158 expressing reporter cells. LAYN-expressing Jurkat cells were co-cultured in a 1:5 ratio with CD16AV158-expressing Jurkat NFAT luciferase reporter cells at 37 °C for 4 h in the presence of an anti-LAYN antibody or a control antibody. BIO-GLO™ (Promega, Madison, WI) was added to the cells and then the luminescence was read for measurement of luciferase activity. The half-maximal effective concentration (EC50) was calculated using GraphPad Prism. Results are shown in Table 25 below and in FIG. 6.

TABLE 25
ADCC reporter assay activity of
Fc-enhanced anti-LAYN antibodies

| | | Max rel. luciferase |
|---|---|---|
| Anti-LAYN mAb | EC₅₀ (pM) | activity |
| A86 | 33.5 | 1 |
| A86 + GAALIE | 15.4 | 1.31 |
| A86 + SDIE | 11.4 | 1.45 |
| A85_afuc1 | 7.09 | 1.09 |
| A85_afuc2 | 19.93 | 1.31 |

These data show that all Fc variants improve the CD16A receptor activity due to crosslinking of CD16A upon binding to LAYN positive cells resulting in increased maximum reporter expression and 2- to 4-fold improved EC50 values as compared to non-modified (wildtype) Fc (A86).

### Example 11: ADCC of Anti-LAYN Fc Variant Antibodies

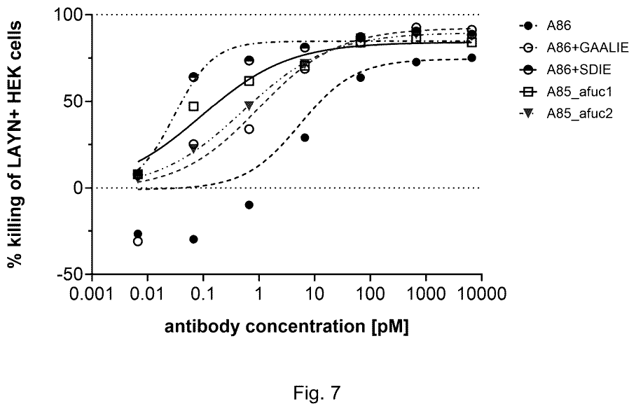

The capacity of the anti-LAYN Fc variant antibodies to induce ADCC-mediated killing of LAYN-expressing cells was evaluated in an impedance-based cell viability assay. CD16AV158-KILR cells (Eurofins Discovery; also referred to as “CD16-KILR cells”) were used as CD16A-expressing effector cells and cultured according to manufacturer's instructions prior to the experiment. In this assay, LAYN-expressing HEK293 cells were seeded in a 384-well CytoView-Z plate (Axion Biosystems) to establish a monolayer. Cell growth and viability was monitored by impedance measurement with the Maestro ZHT (Axion Biosystems). After reaching an impedance equivalent to 50% confluency, CD16-KILR cells were co-cultured with LAYN-expressing HEK cells at a ratio of 1:1 in the presence of titrations of anti-LAYN Fc variant antibodies or an anti-LAYN antibody (A86) having a non-modified (wildtype) IgG1 backbone for up to 24 hours at 37 °C and 5% CO₂. Maximum lysis of LAYN-expressing HEK293 cells was assessed by addition of 0.0025% Tween. The anti-LAYN antibody with the non-modified (wildtype) IgG1 backbone was used to as no lysis control. Impedance was measured over the whole assay period. The area under the curve (AUC) of the impedance (normalized to assays start) is used to calculate % killing. % Killing=(1−(specific lysis−maximum lysis)/(no lysis−maximum lysis))×100. Results are shown in Table 26 below and in FIG. 7.

TABLE 26
ADCC of LAYN-expressing HEK
by anti-LAYN antibodies

| Anti-LAYN mAb | EC₅₀ (pM) | Max Response (%) |
|---|---|---|
| A86 | 5.47 | 75.2 |
| A86 + GAALIE | 0.97 | 91.2 |
| A86 + SDIE | 0.03 | 88.4 |
| A85_afuc1 | 0.09 | 84.0 |
| A85_afuc2 | 0.52 | 87.7 |

These data show that anti-LAYN Fc variant antibodies improve cytotoxicity on LAYN-positive HEK cells resulting in an increase in ADCC—from 75% up to 91%. Additionally, all Fc variants improve the EC50 with a range starting from 5-fold for G236A/A330L/1332E (“GAALIE”) up to 60-fold for afucosylation or S239D/1332E (“SDIE”).

### Example 12: In-Vivo Efficacy of Anti-LAYN Antibodies in MC38-Tumor Bearing Mice

To evaluate the efficacy of anti-LAYN antibodies in syngeneic mouse tumor models, a human LAYN knock-in mouse model was generated, since expression of the murine homolog Layn on tumor-infiltrating regulatory T cells was not detectable. C57BL/6N-Foxp3tm5 (hLAYN) Geno (alias huLAYN mice) were generated by genetically modifying C57BL/6N mice with a knock in of the extracellular domain (ECD) of the human Layilin isoform 2 CDS (huLAYN) consisting of the amino acids 1-250 in the FoxP3 locus (LAYN v2 ECD1-250; SEQ ID NO: 195; see Example 1). Expression is coupled to FoXP3 expression via an IRES.

To demonstrate anti-tumor efficacy in mice, anti-LAYN antibodies with mouse IgG2a constant region (moA85, moA1, moA2) were used. Antibody moA85 combines the CDR and VH/VL sequences of antibody A85 as described above with mouse IgG2a constant regions (moA85 HC: SEQ ID NO: 175; moA85 LC: SEQ ID NO: 176). Similarly, antibodies moA1 and moA2 combine the CDR and VH/VL sequences of antibodies A1 and A2, respectively, as described above, with mouse IgG2a constant regions (same mouse IgG2a constant regions as in moA85). Accordingly, antibodies moA85, moA1 and moA2 differ only in their CDR and VH/VL sequences, which correspond to those of A85, A1 and A2, respectively. The mouse IgG2a antibodies were not afucosylated. An MC38 tumor model was used to model tumors with a relevant degree of immune infiltration and dependency on regulatory T cells to suppress the anti-tumoral immune response.

In brief, a suspension containing 1×10⁶ tumor cells per 100 μL PBS was subcutaneously injected into the flank of female and male immunocompetent huLAYN mice. At a mean tumor size of at least 80 mm³ anti-LAYN antibody was administered once by intraperitoneal injection in a range between 0.1 mg/kg to 10 mg/kg in a volume of 10 mL/kg or by intravenous injection in a range between 0.1 mg/kg to 10 mg/kg in a volume of 10 ml/kg. Non-binding antibodies of the respective isotype served as control. Groups comprised of n=12 or n=11 mice, respectively.

Tumor size and body weight was recorded three times per week. Tumor caliper measurements were converted into tumor volumes using the formula: volume=A (length×width×height). Tumor growth data are plotted as mean volume over time. Median tumor growth inhibition was calculated relative to isotype control treated group at the last day with 70% of control animals remaining in study. On study, mice received sterile rodent chow and water ad libitum and were housed in sterile filter-top cages with 12-h light/dark cycles. All mouse experiments were conducted in accordance with the guidelines of the Association for Assessment and Accreditation of Laboratory Animal Care International.

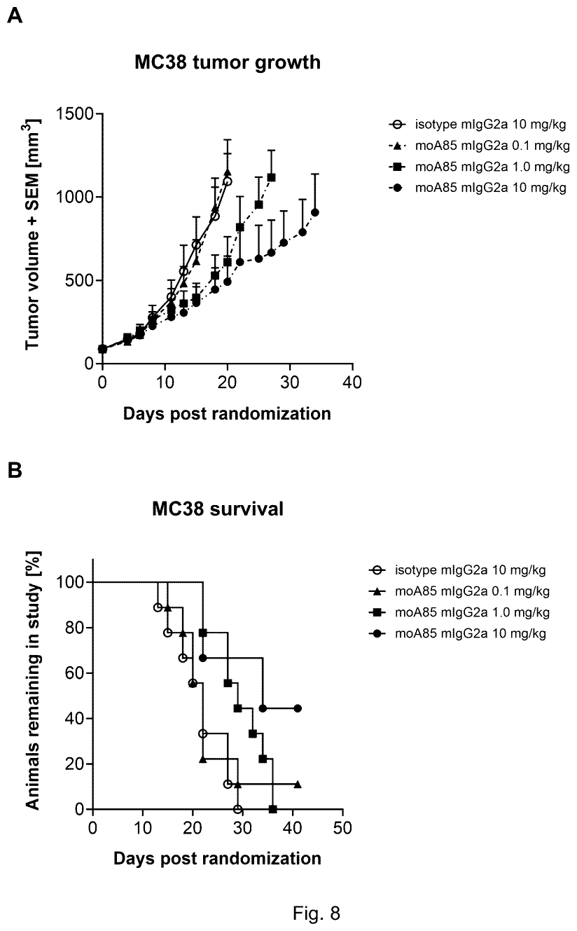

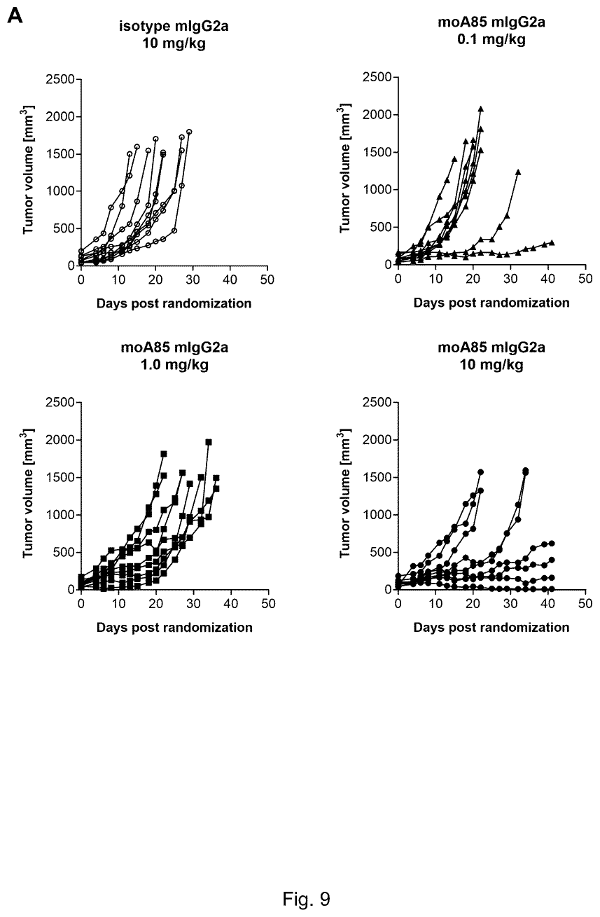

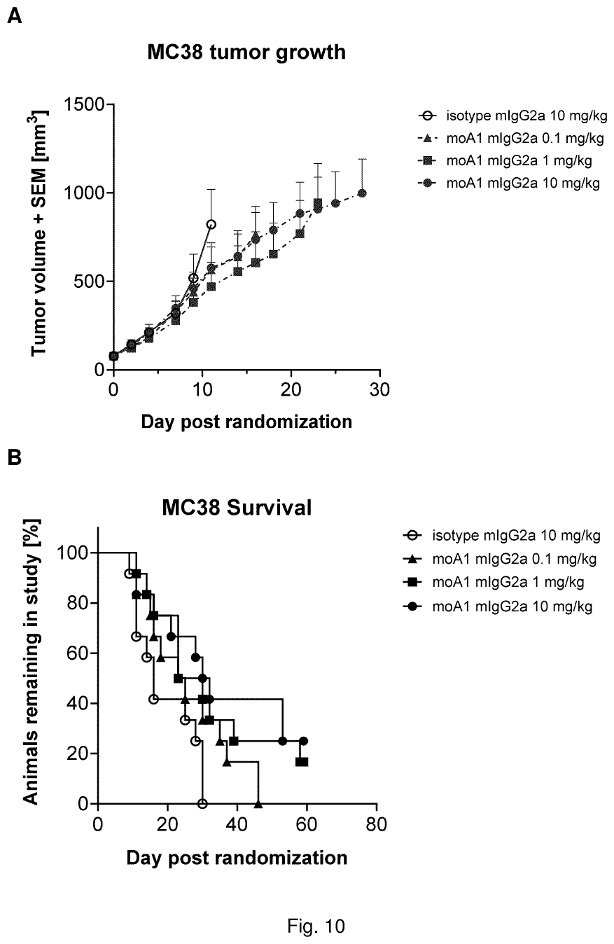

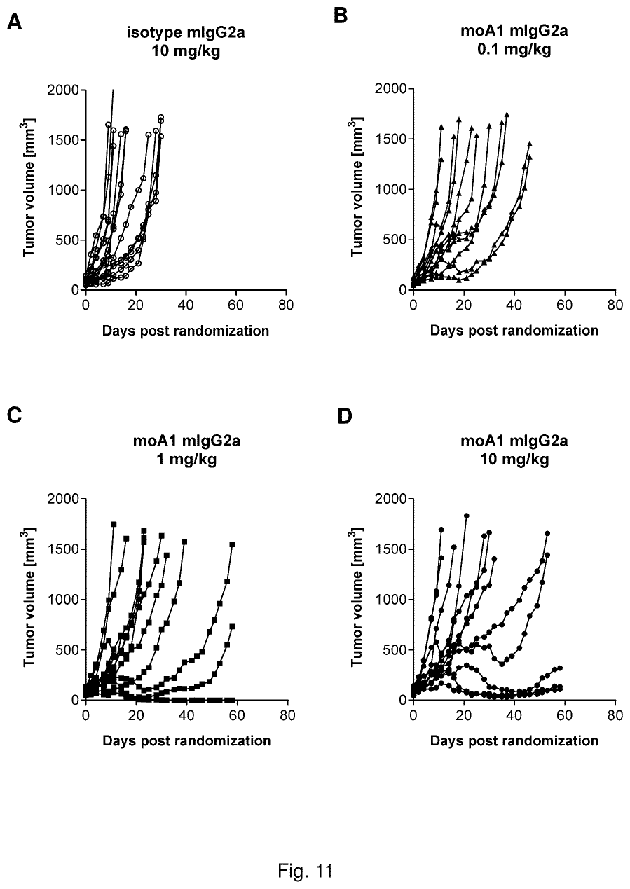

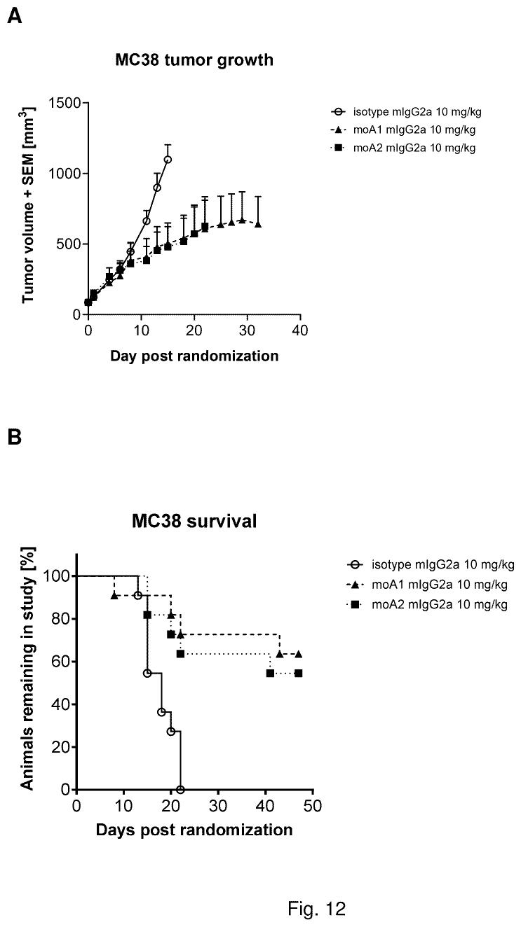

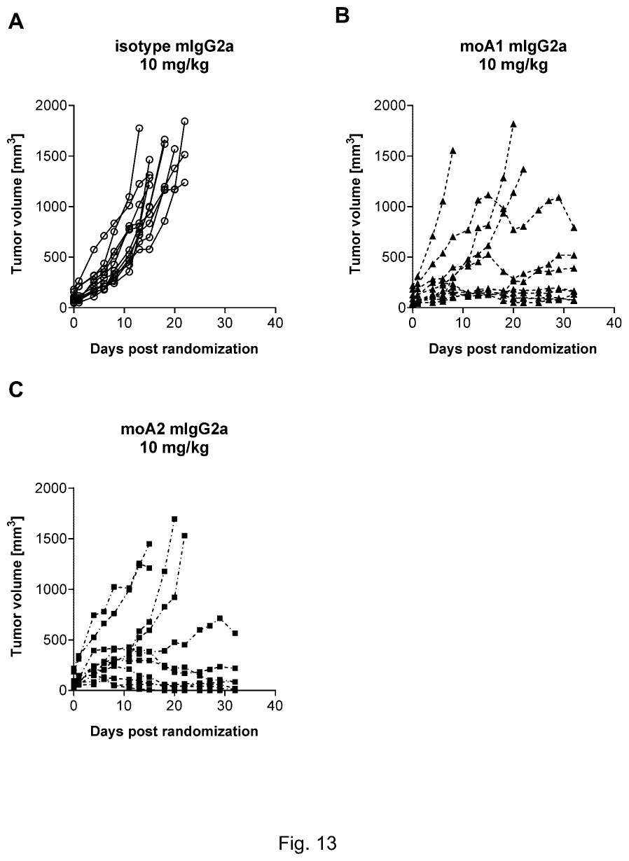

Results are shown in FIGS. 8-13. FIG. 8 shows (A) the MC38 tumor growth and (B) survival for the different dose groups treated once intraperitoneally with moA85 and isotype control. FIG. 9 shows the individual tumor growth data for each group (A: control; B: 0.1 mg/kg moA85; C: 1.0 mg/kg moA85; D: 10 mg/kg moA85). FIG. 10 shows (A) the MC38 tumor growth and (B) survival for the single intravenous treatment with different doses of moA1 or isotype control. FIG. 11 shows the individual tumor growth data for each treatment group (A: control; B: 0.1 mg/kg moA1; C: 1 mg/kg moA1; D: 10 mg/kg moA1). FIG. 12 shows (A) the MC38 tumor growth and (B) survival for the intravenous treatment with 10 mg/kg of either moA1 or moA2 versus control. FIG. 13 shows the individual tumor growth for each group (A: control; B: 10 mg/kg moA1; C: 10 mg/kg moA2).

Treatment with anti-LAYN antibody moA85 and moA1 showed dose-dependent anti-tumor efficacy with increased survival in MC38 tumor-bearing mice after a single administration. Treatment with moA85 showed no anti-tumor efficacy at the lowest dose of 0.1 mg/kg, but increased survival and median tumor growth inhibition (TGI) of 54% and 72% was observed at a dose of 1 mg/kg and 10 mg/kg, respectively. Treatment with moA1 resulted also in increased survival at a dose of 1 and 10 mg/kg and improved median TGI of 52%, 57% and 59% at day 14 at a dose of 0.1 mg/kg, 1 mg/kg and 10 mg/kg, respectively. Treatment with anti-LAYN antibodies moA1 and moA2 at a dose of 10 mg/kg resulted in significantly increased survival and median TGI of 70% and 87%, respectively.

### Example 13: Anti-LAYN Antibody Mediate Depletion of LAYN+ Regulatory T Cells

In the huLAYN mice of the MC38 tumor model described in Example 12, the mode-of-action was characterized, namely Treg depletion within the tumor depending on ADCC (antibody-dependent cellular cytotoxicity) and ADCP (antibody-dependent cellular phagocytosis).

To this end, tumor and tissue samples were taken from identically treated animals (n=5-10 per group) either at 3 days, 7 days and 14 days or only 7 days after a single antibody administration and were analyzed by flow cytometry after dissociation into single cells and lysis of erythrocytes with particular focus on changes in absolute numbers of regulatory T cells and CD8+ effector T cells.

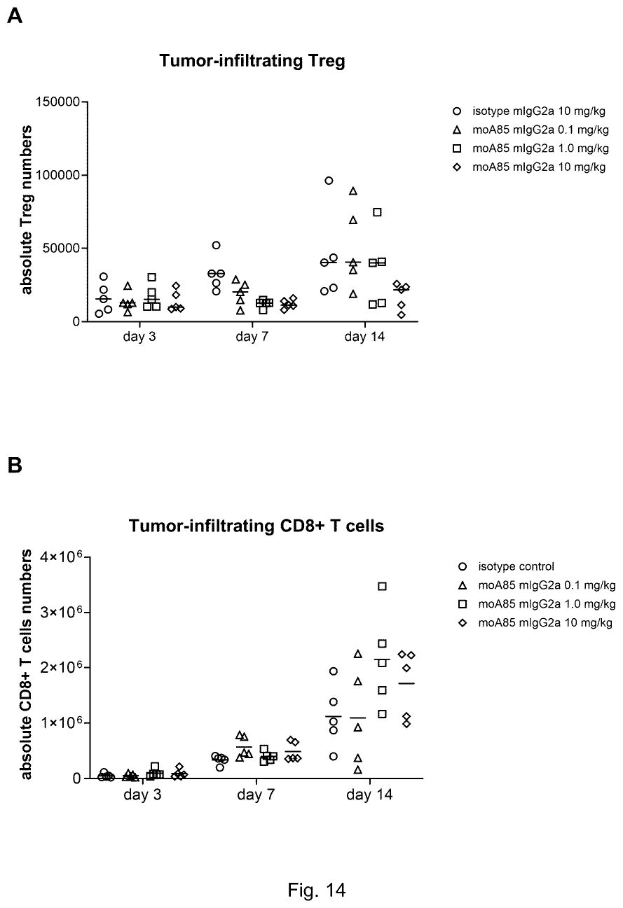

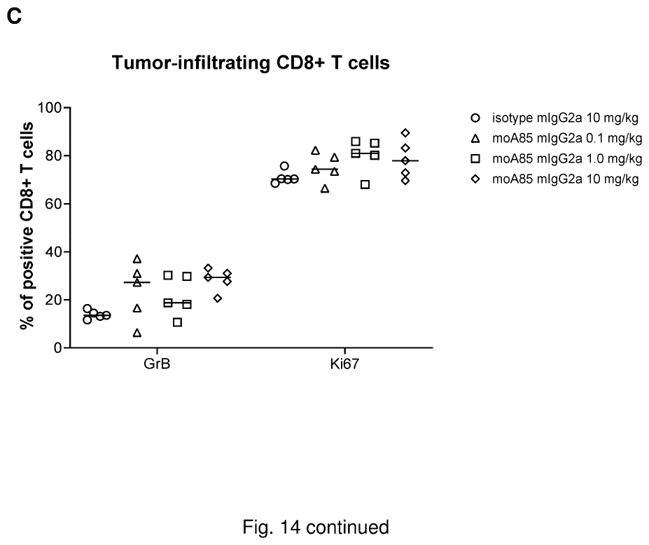

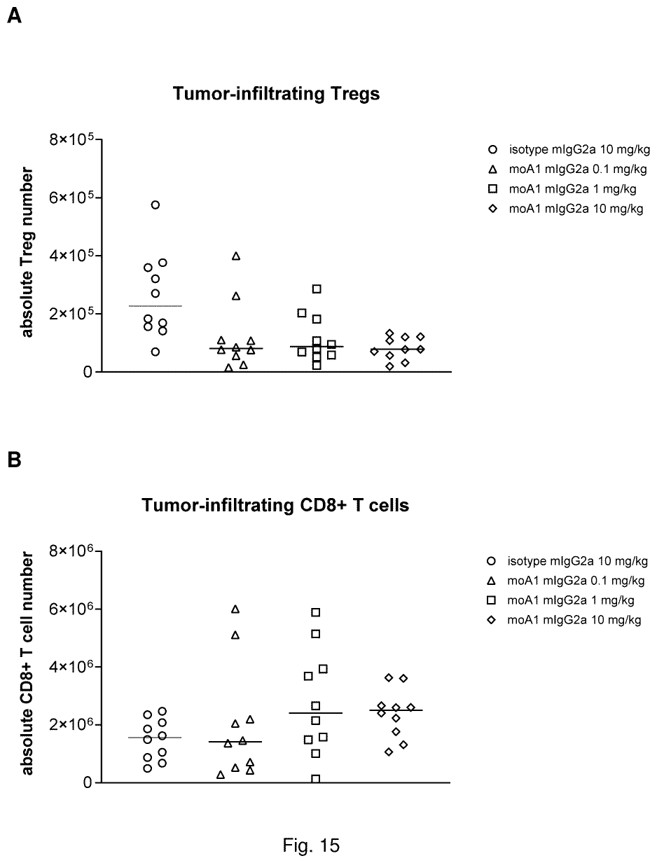

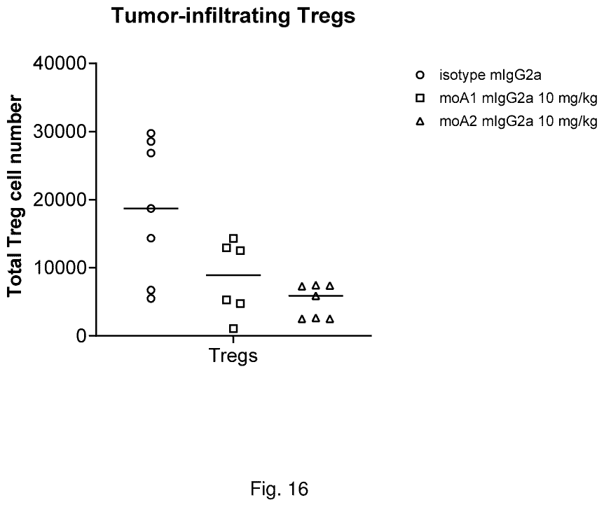

Results for treatment (n=5 per group) with 0.1, 1 or 10 mg/kg of moA85 at day 3, 7 and 14 are shown in FIG. 14 with tumor-infiltrating Tregs (A) and tumor-infiltrating CD8-positive T cells (B: absolute CD8+ T cell numbers; C: % of positive CD8+ T cells). Results for animals (n=10 per group) treated with different doses of moA1 (0.1, 1 or 10 mg/kg) are shown in FIG. 15 with tumor-infiltrating Tregs (A) and tumor-infiltrating CD8-positive T cells (B) at day 7 post intravenous administration. Finally, results for a single dose of 10 mg/kg of either moA1 or moA2 (n=7 per group) are shown in FIG. 16 with tumor-infiltrating Tregs at day 7 post intravenous administration.

Treatment of huLAYN mice harboring MC38 tumors with anti-LAYN antibodies drastically reduced the number of FoxP3+ regulatory T cells expressing LAYN, compared to mice treated with the isotype control. The observed reduction in regulatory T cells was dose-dependent and durable after a single administration of anti-LAYN antibodies. The reduction in FoxP3+ regulatory T cells was followed by an increase in tumoral CD8+ T cells and results in increased expression of Granzyme B (GrB) and Ki67 PD1 at day 14 after treatment start.

### Example 14: Efficacy of Anti-LAYN Antibodies in Combination Therapy

To analyze the efficacy of anti-LAYN antibodies alone or in combination with checkpoint targeting antibodies such as anti-PD1, two different syngeneic tumor models were used. The used models deviate in their response to treatment with known checkpoint-inhibitors like anti-PD1.

The anti-tumor activity of anti-LAYN antibodies moA85 and moA1 (described above) was assessed as a single agent or in combination with anti-murine PD1 (anti-mPD1) antibody mIgG1-D265A Ab (Invivogen). Antibodies were administered once per week for 2 (moA85) or 4 (moA1) consecutive weeks after randomization. In the MC38 and MB49 tumor model 1 mg/kg and 10 mg/kg of each antibody was administered, respectively.

For the MC38 and MB49 tumor model, a suspension containing 1×10⁶ or 5×10⁵ tumor cells per 100 μL PBS was subcutaneously injected into the flank of female and male immunocompetent huLAYN mice, respectively. At a mean tumor size of at least 69 mm³ anti-LAYN antibody moA85, moA1 or murine anti-PD1 mIgG1D265A antibody (Invivogen) was administered by intraperitoneal injection at a dose of 1.0 mg/kg (MC38) or 10 mg/kg (MB49) in a volume of 10 mL/kg in PBS. Non-binding antibodies of the respective isotype served as control. Groups comprised of n=12 mice. For combination, (i) anti-LAYN antibody moA85 or moA1 and (ii) anti-PD1 antibody mIgG1D265A (Invivogen) were administered at a dose of 1.0 mg/kg (MC38) or 10 mg/kg (MB49) in a volume 10 ml/kg separately at about the same time. Animal treated with monotherapy received only the respective (single) therapeutic antibody.

Tumor size and body weight was recorded three times per week. Tumor caliper measurements were converted into tumor volumes using the formula: volume=A (length×width×height). Tumor growth data are plotted as mean volume over time. On study, mice received sterile rodent chow and water ad libitum and were housed in sterile filter-top cages with 12-h light/dark cycles. All mouse experiments were conducted in accordance with the guidelines of the Association for Assessment and Accreditation of Laboratory Animal Care International.

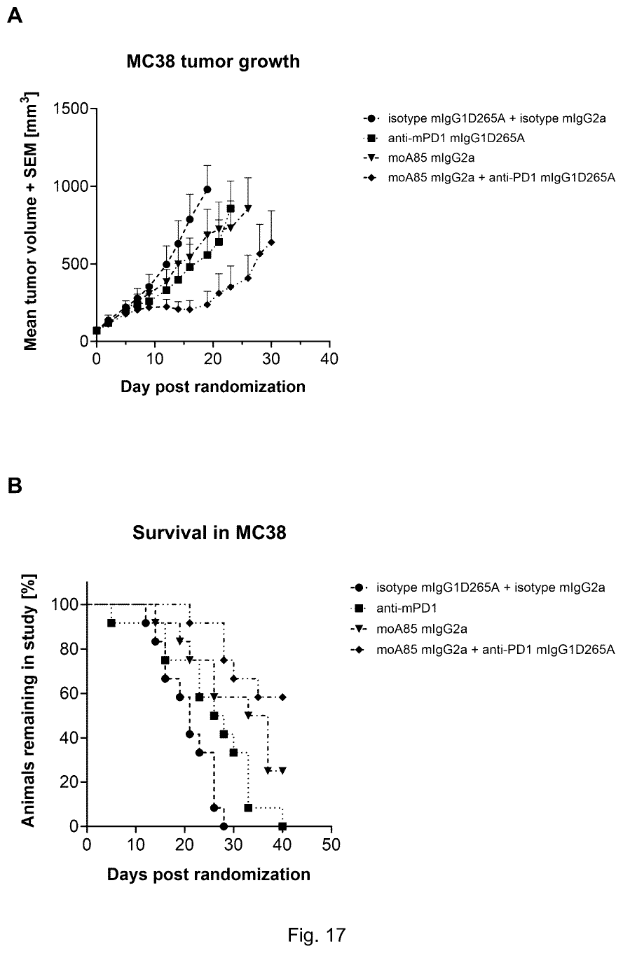

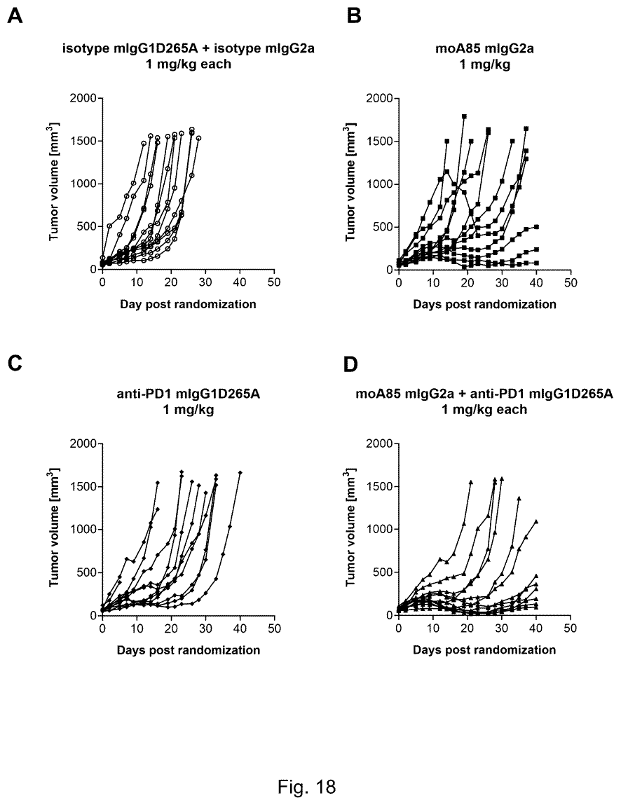

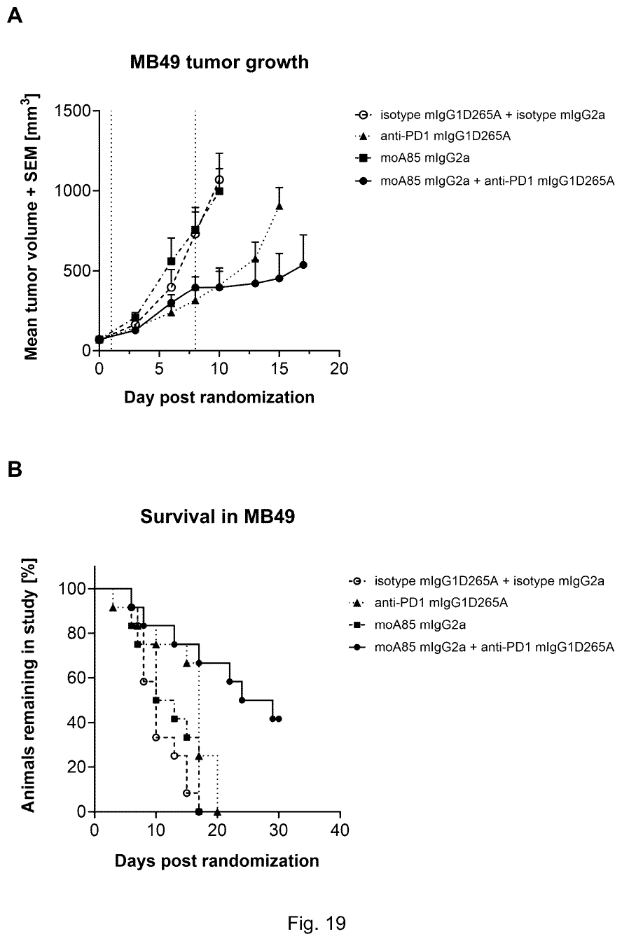

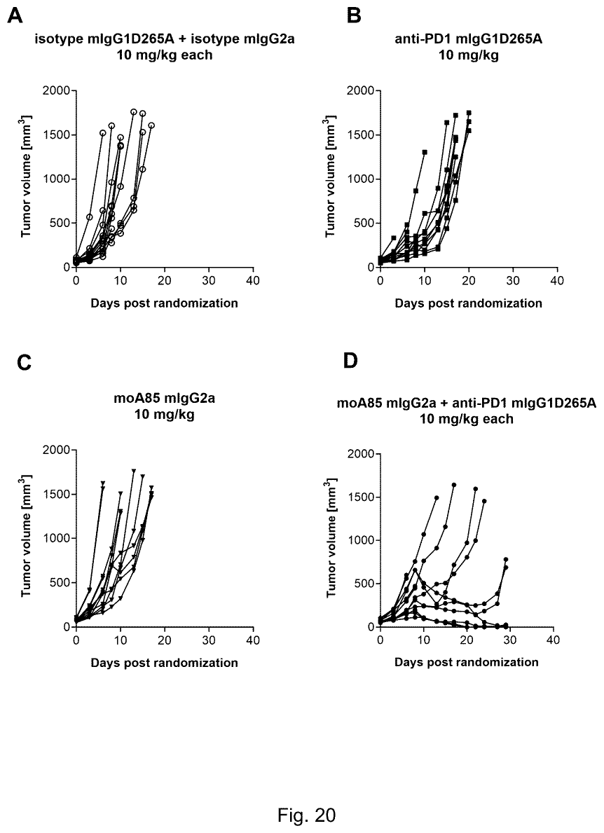

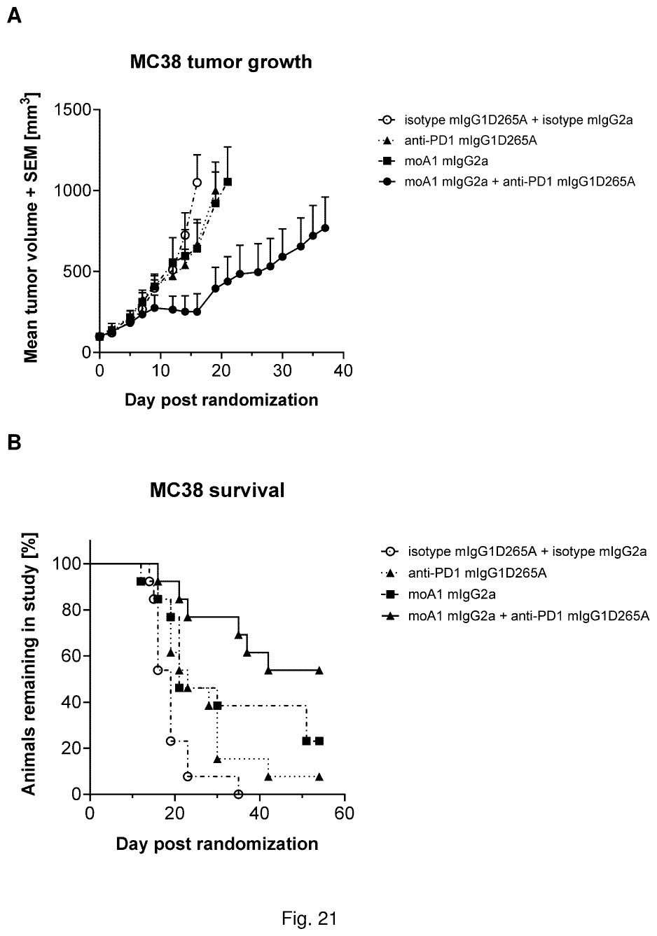

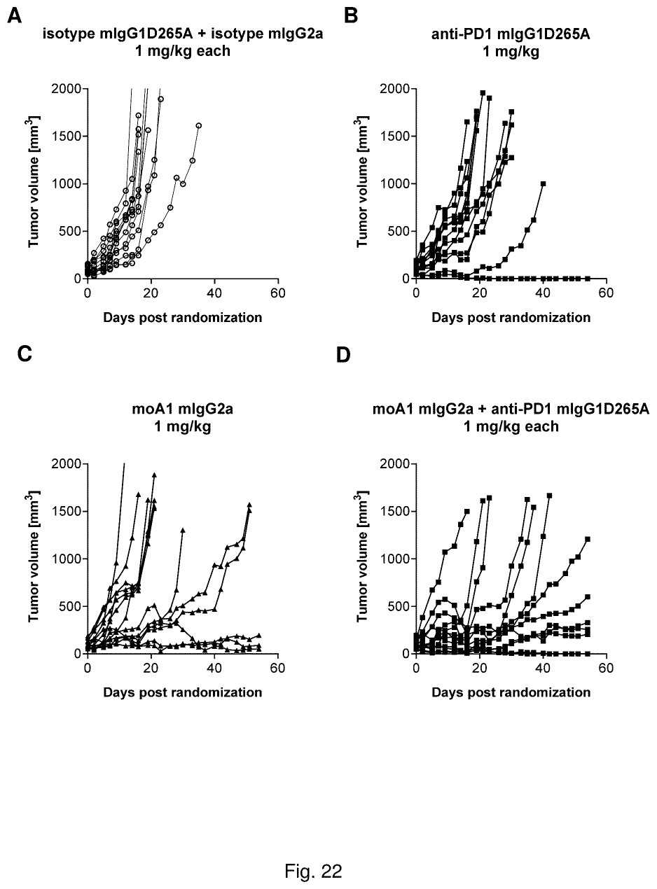

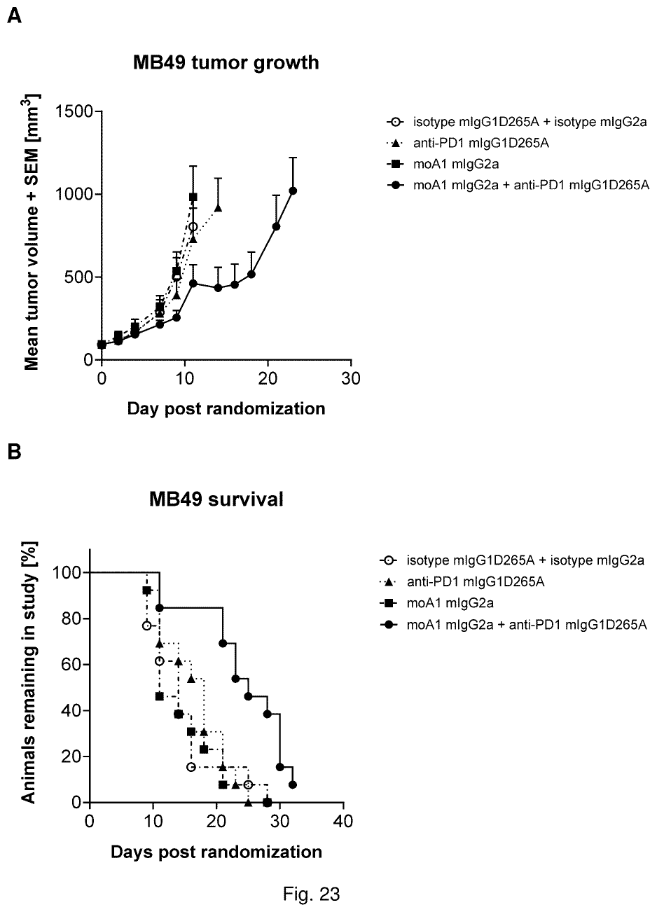

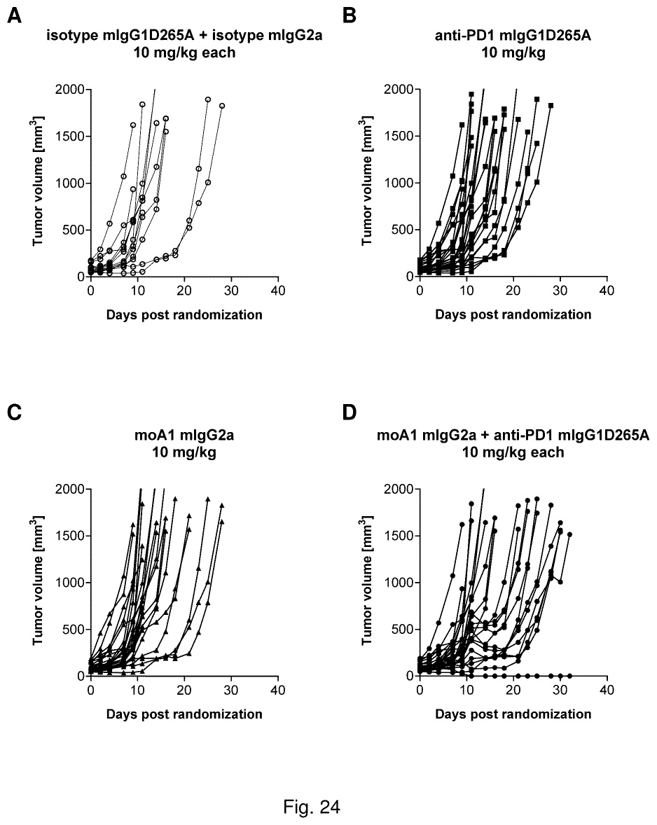

Results are shown in FIGS. 17-24. FIG. 17 shows (A) tumor growth and (B) survival for the different combination groups with moA85 in the MC38 tumor model. FIG. 18 shows the data for the different animals per group in the MC38 tumor model (A: control; B: 1.0 mg/kg moA85; C: 1.0 mg/kg anti-PD1; D: 1.0 mg/kg moA85 combined with 1.0 mg/kg anti-PD1). FIG. 19 shows (A) tumor growth and (B) survival for the different combination groups with moA85 in the MB49 tumor model. FIG. 20 shows the data for the different animals per group in the MB49 tumor model (A: control; B: 10 mg/kg anti-PD1; C: 10 mg/kg moA85; D: 10 mg/kg moA85 combined with 10 mg/kg anti-PD1). FIG. 21 shows (A) tumor growth and (B) survival for the different combination groups with moA1 in the MC38 tumor model. FIG. 22 shows the data for the different animals per group in the MC38 tumor model (A: control; B: 1.0 mg/kg anti-PD1; C: 1.0 mg/kg moA1; D: 1.0 mg/kg moA1 combined with 1.0 mg/kg anti-PD1). FIG. 23 shows (A) tumor growth and (B) survival for the different combination groups with moA1 in the MB49 tumor model. FIG. 24 shows the data for the different animals per group in the MB49 tumor model (A: control; B: 10 mg/kg anti-PD1; C: 10 mg/kg moA1; D: 10 mg/kg moA1 combined with 10 mg/kg anti-PD1).

The data show that the combination of both anti-LAYN antibodies with anti-PD1 resulted in a strong synergistic interaction evidenced by a massive enhancement of anti-tumor activity independent of the intrinsic PD-1 sensitivity of the employed syngeneic mouse tumor model.

### Example 15: Anti-LAYN Antibodies Deplete Human Tumor Tregs Ex Vivo

To investigate ADCC of human tumor Tregs isolated from dissociated non-small cell lung cancer (NSCLC) tumor tissue, donor natural killer (NK) cells were isolated and activated using 500 U/ml of IL-2 (PeproTech) for 24 h. Frozen dissociated human non-small cell lung cancer tissue was thawed and co-cultured ex vivo in a 1:1 ratio with activated allogeneic NK cells for 16 h in the presence of anti-LAYN antibodies A1 or A86_afuc or an isotype control mAb. The cells were stained and Treg depletion was measured by flow cytometry using FlowJo software to analyze the presence of defined cell populations.

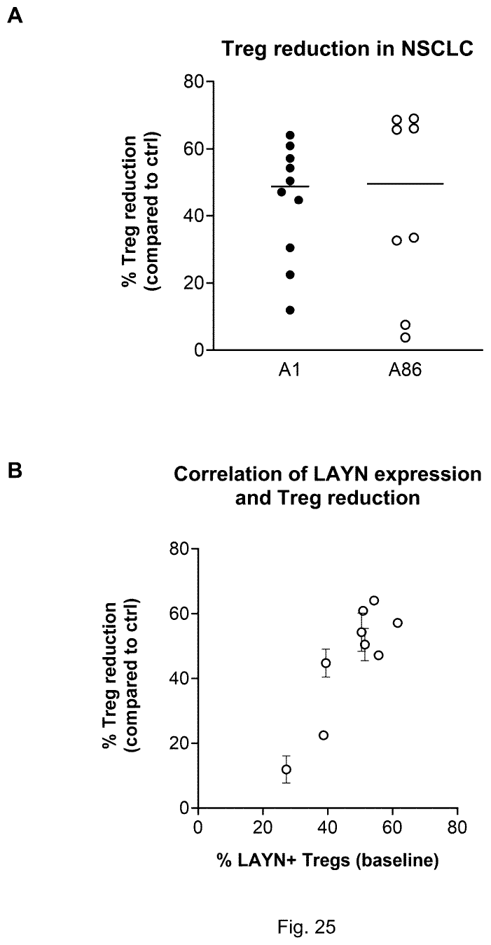

Results are shown in FIG. 25. FIG. 25 (A) shows the reduction in human tumor-resident regulatory T cells ex vivo with A1 or A86 compared to the corresponding isotype control treated sample. (B) shows the correlation between Treg reduction with A1 and LAYN expression in tumor Tregs.

The data show that anti-LAYN antibodies A1 and A86 reduce drastically human tumor-resident regulatory T cells ex vivo isolated from human lung cancer patients resulting in a median reduction of 48.8% or 49.6% respectively. This closely correlates with the expression of LAYN in tumor-resident regulatory T cells.

### Example 16: Epitope/Paratope: Cryo-EM Structure of LAYN in Complex with Fab A85

To study the structure of the anti-LAYN antibody bound to LAYN and to identify the respective epitope/paratope, the three-dimensional structure of LAYN in complex with Fab A85 was determined by cryo electron microscopy (cryo-EM) to 2.4 Å resolution. The paratope and epitope were determined based on the observed interface between Fab and antigen. In addition, the LAYN-Ca²⁺ binding site was confirmed to be non-overlapping with the epitope of the anti-LAYN antibody.

Fab A85 differs from antibody A85 described above only in that it lacks the CH2 and CH3 domains of the heavy chain Fc region. Accordingly, Fab A85 has the heavy chain (HC) sequence of SEQ ID NO: 177, while the CDR sequences, VH and VL sequences as well as the light chain (LC) sequence correspond to those of antibody A85.

To increase the size of the LAYN-v2-no-Tag-Fab A85 complex, an additional Fab was used (Fab X), which is assumed to bind to LAYN-v2-no-Tag (SEQ ID NO: 191) as well, but to a different epitope (close to the C-terminus of LAYN-v2-no-Tag).

Fab A85 and Fab X were produced according to standard techniques. For complex formation, both Fabs and LAYN-v2-no-Tag (SEQ ID NO: 191) were incubated together in 20 mM Tris pH 8, 150 mM NaCl, 1 mM CaCl₂), at 1 mg/ml each, leading to a molar LAYN-v2-no-Tag excess of 2:1:1 with respect to each Fab. The mixture was incubated for 4 h at RT, followed by high-speed centrifugation (14000 rpm×5 min) and SEC isolation of the complex by Superdex200 10/300 Increase using SEC buffer: 20 mM Tris pH 8, 150 mM NaCl, 1 mM CaCl₂).

The purified complex was concentrated to 2.0 mg/ml in 20 mM Tris pH 8, 150 mM NaCl, 1 mM CaCl₂) and used for grid preparation and Cryo-EM data collection.

The structure of LAYN-v2-no-Tag in complex with Fab A85 is shown in FIG. 26. The observed structure is composed of LAYN-v2-no-Tag and Fab A85 at a 1:1 stoichiometry. Fab X was not observed in the complex.

The interface between LAYN-v2-no-Tag and Fab A85 was determined using the “Protein Interaction Analysis” module after using the automated “Protein Preparation” workflow (Maestro Version 13.8.135, MMshare Version 6.4.135, Release 2023-4, Platform Windows-x64; Schroedinger, Inc) with a cut-off distance of 4.0 Å. The majority of interaction distances shown are below 3.0 Å. Results are shown in FIG. 27 and Table 27 below.

TABLE 27
Residues in LAYN (SEQ ID NO: 191) and Fab A85 (H: heavy chain, SEQ
ID NO: 177; L: light chain, SEQ ID NO: 81) having a distance
of no more than 4.0 Å and specific interactions by cyro-EM

| LAYN | Fab A85 | | Specific | |
|---|---|---|---|---|
| Residues | Residues | Distance(A) | Interactions | # H-Bond |
| A:GLN 138 | L:TRP 32 | 2.6 A | | 0 |
| A:GLN 138 | L:THR 31 | 3.8 A | | 0 |
| A:PHE 137 | H:GLY 102 | 2.6 A | | 0 |
| A:PHE 137 | H:TYR 101 | 2.2 A | | 0 |
| A:MET 136 | L:TRP 32 | 2.7 A | | 0 |
| A:MET 136 | H:GLY 102 | 2.6 A | | 0 |
| A:MET 136 | H:ARG 103 | 2.3 A | | 0 |
| A:MET 136 | H:TYR 101 | 2.5 A | | 0 |
| A:TYR 135 | H:ARG 103 | 3.9 A | | 0 |
| A:PRO 134 | L:TRP 32 | 2.7 A | | 0 |
| A:PRO 134 | H:ARG 103 | 2.8 A | | 0 |
| A:PRO 134 | L:LEU 91 | 3.0 A | | 0 |
| A:PRO 134 | L:TYR 92 | 2.5 A | | 0 |
| A:PRO 134 | H:GLY 102 | 3.7 A | | 0 |
| A:GLY 133 | L:TYR 92 | 2.4 A | | 0 |
| A:GLY 133 | L:TRP 32 | 3.7 A | | 0 |
| A:GLY 132 | L:TYR 92 | 2.8 A | | 0 |
| A:GLY 132 | L:TRP 32 | 2.7 A | | 0 |
| A:ILE 131 | L:TYR 92 | 2.6 A | | 0 |
| A:ALA 129 | H:ARG 103 | 3.2 A | | 0 |
| A:ALA 129 | L:TYR 92 | 2.8 A | | 0 |
| A:ALA 129 | L:SER 93 | 3.1 A | | 0 |
| A:ALA 129 | L:THR 94 | 3.8 A | | 0 |
| A:PRO 128 | H:ARG 103 | 3.0 A | | 0 |
| A:ALA 127 | H:ARG 103 | 2.2 A | 2x hb to H:ARG 103 | 20 |
| A:ALA 127 | H:TRP 33 | 2.9 A | | 0 |
| A:SER 126 | H:TYR 52 | 2.6 A | | 0 |
| A:SER 126 | H: TRP 33 | 2.7 A | | 0 |
| A:SER 126 | H:SER 55 | 2.8 A | | 0 |
| A:SER 126 | H:SER 57 | 4.0 A | | 0 |
| A:PRO 125 | H:ARG 103 | 3.1 A | | 0 |
| A:PRO 125 | H:TRP 33 | 2.1 A | 1x hb to H:TRP 33 | 10 |
| A:PRO 125 | H:TYR 52 | 2.5 A | | 0 |
| A:PRO 125 | H:TYR 101 | 2.8 A | | 0 |
| A:PRO 125 | H:SER 31 | 3.6 A | | 0 |
| A:GLN 124 | H:ARG 103 | 2.8 A | | 0 |
| A:HIS 123 | H:TYR 101 | 2.1 A | 1x hb to H:TYR 101 | 10 |
| A:VAL 108 | L:TYR 92 | 2.9 A | | 0 |
| A:PRO 67 | H:TYR 101 | 2.6 A | | 0 |
| A:LEU 66 | H:SER 31 | 2.4 A | | 0 |
| A:LEU 66 | H: TYR 32 | 2.9 A | | 0 |
| A:LEU 66 | H:THR 28 | 2.3 A | | 0 |
| A:LEU 66 | H:TYR 101 | 2.5 A | | 0 |
| A:GLU 63 | H:TYR 101 | 2.5 A | | 0 |
| A:GLU 63 | H: TYR 32 | 2.1 A | 1x hb to H:TYR 32 | 10 |
| A:GLU 63 | H:ARG 98 | 3.9 A | | 0 |
| A:ILE 62 | H:TYR 101 | 2.2 A | | 0 |
| A:GLU 59 | H:ARG 100 | 4.0 A | | 0 |
| A:LYS 56 | L:TYR 49 | 2.3 A | | 0 |
| A:LYS 56 | L:SER 53 | 2.6 A | | 0 |
| A:LYS 56 | L:ALA 50 | 3.3 A | | 0 |
| A:GLU 52 | L:SER 53 | 3.3 A | | 0 |

It was found that hydrogen bonds are formed between LAYN-Ala127 and Fab A85 HC-Arg103, LAYN-Pro125 and Fab A85 HC-Trp33, LAYN-His123 and Fab A85 HC-Tyr101 and LAYN-Glu63 and Fab A85 HC-Tyr32. The positions in the Fab heavy chain are those in SEQ ID NO: 177 (HC Fab A85), which correspond to those in SEQ ID NO: 143 (VH A85). Accordingly, all four amino acid residues of the Fab, which are involved in direct hydrogen bonds with LAYN, are located in the HCDR1 and HCDR3, namely Tyr32 and TRP33 in HCDR1 as well as Tyr101 and Arg103 in HCDR3. For illustration, they are shown in bold/underlined in the respective sequences of HCDR1 and HCDR3, respectively:

(HCDR1 A85):
SEQ ID NO: 1
SYWMD
(HCDR3 A85):
SEQ ID NO: 142
ERYGRNAMDY

These amino acids are maintained in the HCDR1 and HCDR3 sequences of all exemplary anti-LAYN antibodies described herein (e.g., in Example 2), which show specific binding to LAYN (antibodies P and A1-A97).

The data in the Table 27 and in FIG. 27 show that the epitope of LAYN, to which Fab A85 binds, involves primarily the loop of LAYN isoform 2 containing residues 123-138 of SEQ ID NO: 191, which directly interacts with the heavy chain of the Fab (FIG. 27A), and the helix of LAYN isoform 2 containing residues 52-67 of SEQ ID NO: 191, which interacts in part directly with the heavy chain of Fab A85, in part via ordered water molecules with the light chain of Fab A85 (FIG. 27B). As shown in FIG. 27C and Table 27 above, residues E52, K56, E59, 162, E63, L66, P67, V108, H123, Q124, P125, S126, A127, P128, A129, I131, G132, G133, P134, Y135, M136, F137 and Q138 of SEQ ID NO: 191 (ECD LAYN isoform 2) have a distance of no more than 4.0 Å to one or more amino acid residues of Fab A85. This distance is assumed to include the potential polar and non-polar interactions between the respective LAYN and Fab residues.

Without being bound to any theory, it is assumed that human LAYN isoform 1 and isoform 2 are identical regarding their potential interaction surface (see also FIG. 1A with boxes indicating those sections of LAYN isoforms 1 and 2, which correspond to residues 52-67 and 123-138 of SEQ ID NO: 191). Accordingly, while human LAYN isoform 2 has been used in this example, it is assumed that the antibody would bind to the corresponding epitope (interaction surface) in human LAYN isoform 1.

The predicted calcium binding site of the LAYN-protein has been determined, where the calcium ion is coordinated by Glu110, Ser112, Glu116, Asn140, Asp141, and a water molecule. The calcium binding site is not in proximity to the paratope (distance to Fab>14 Å).

### Example 17: Competition of Anti-LAYN Antibodies with Collagen Binding

Competition of LAYN antibodies with collagen IV was measured with Bio-layer interferometry (BLI) on an Octet QKe instrument. Running buffer was 50 mM Tris pH 8, 150 mM NaCl, 0.1% Acetic Acid, 0.2% BSA, 0.05% Tween20 and 1 mM CaCl₂). Biotinylated LAYN-v2-His was immobilized on SAX biosensors followed by collagen IV (Abcam ab7536; stock 6 mM in 3% Acetic Acid) binding at 200 nM concentration and antibodies A1-A11, A85_afuc2, A86 as well as a non-binding control Antibody (aTNP). Collagen IV was applied in saturation compared to LAYN-biotin (Collagen IV concentration 20-times>K_{D}) in this experiment, in order to ensure all binding sites of LAYN are occupied. In addition, collagen binding to SAX biosensors without LAYN was analyzed. No unspecific signal of CollagenIV was obtained.

In addition, also competition of anti-LAYN antibodies with collagen IV was measured for the following antibodies: ML1Std (Glasgow J E, Byrnes J R, Barbee S D, Moreau J M, Rosenblum M D, Wells J A. Identifying and antagonizing the interactions between layilin and glycosylated collagens. Cell Chem Biol. 2022 Apr. 21; 29(4):597-604.e7. doi: 10.1016/j.chembiol.2022.01.003. Epub 2022 Feb. 1), 4C11 (Novus Biologicals) and 3F7D7E2 (Sino Biological).

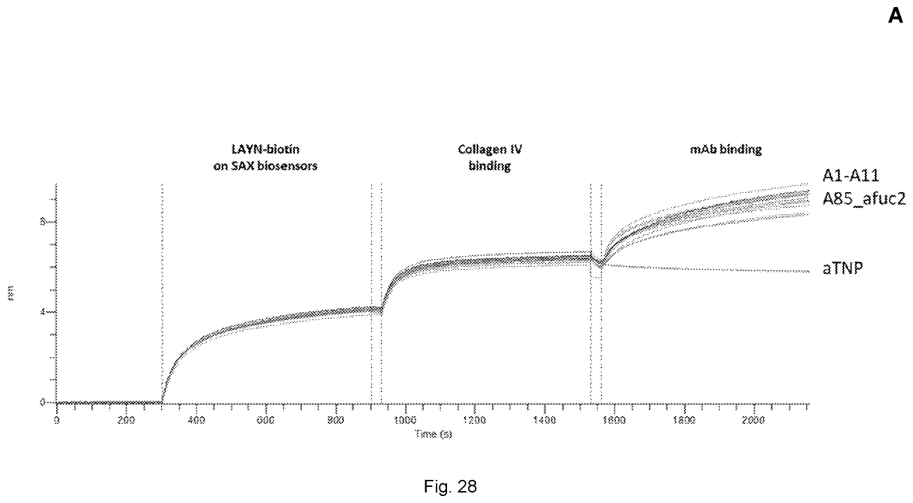

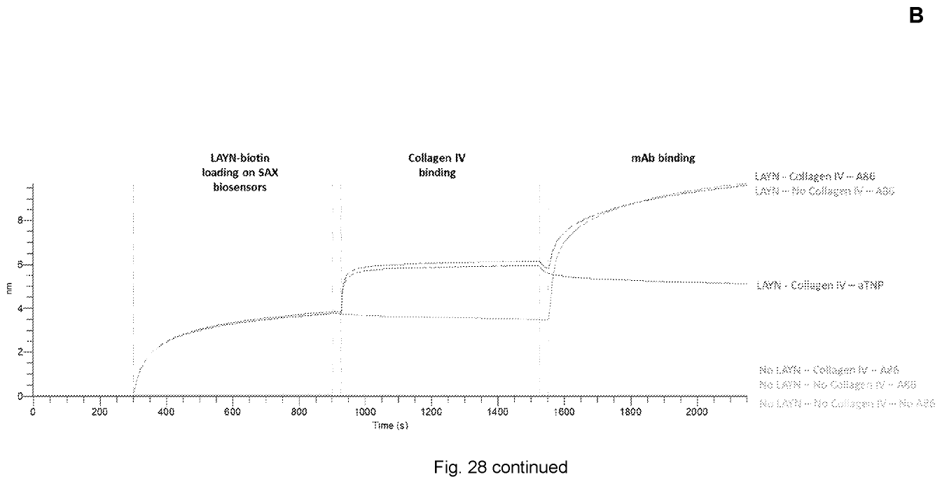

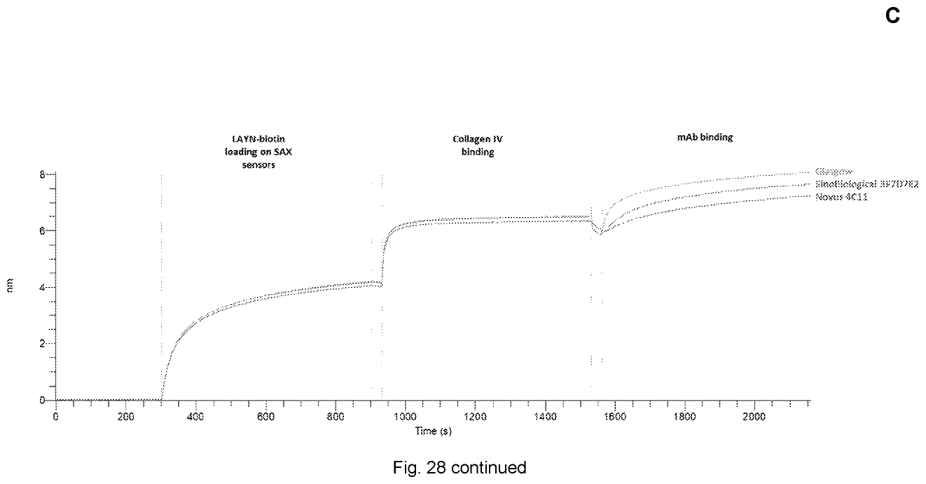

Results are shown in FIG. 28. The data in FIG. 28A show that after collagen IV bound to LAYN, all antibodies (A1-A11 and A85_afuc2) with exception of the control antibody (aTNP) were able to bind to LAYN in the presence of collagen. These data indicate that the antibodies of the present disclosure do not compete or interfere with the binding of collagen IV to LAYN. FIG. 28B shows the data for A86 and additional controls as indicated, e.g., w/wo (with/without) CollagenIV and w/wo LAYN protein. FIG. 28C shows the data for antibodies ML1Std (“Glasgow”), 4C11 and 3F7D7E2. The data in FIG. 28C show that after collagen IV bound to LAYN antibodies ML1Std, 4C11 and 3F7D7E2 were able to bind to LAYN in the presence of collagen. These data indicate that also these antibodies do not compete or interfere with the binding of collagen IV to LAYN. Controls were run in the same manner as shown in FIG. 28B, but are not depicted again in FIG. 28C.

### Example 18: Comparison of Anti-LAYN Antibodies with Regard to Stability and Binding Activity

For comparison, the following anti-LAYN antibodies of the prior art were used:
HL2E8 as described in WO 2022/246085. According to SEQ ID NO: 4 (HL2E8 LCDR1) of WO 2022/246085, the amino acid at position 2 of SEQ ID NO: 4 may be A or T. Therefore, two versions of this antibody were used having either A or T at said position (referred to as “HL2E8_A” and “HL2E8_T”, respectively). In addition, HL2E8 was used in human and mouse IgG1 backbone. HL2E8_A (with huIgG1 backbone) corresponds to HL2Dir described in Glasgow et al., 2022 (Glasgow J E, Byrnes J R, Barbee S D, Moreau J M, Rosenblum M D, Wells J A. Identifying and antagonizing the interactions between layilin and glycosylated collagens. Cell Chem Biol. 2022 Apr. 21; 29(4):597-604.e7. doi: 10.1016/j.chembiol.2022.01.003. Epub 2022 Feb. 1).
mouse-directed antibody ML1Std and human-directed antibody HL1Std described in Glasgow et al., 2022;
commercially available antibody 4C11 (Novus Biologicals);
commercially available antibody 3F7D7E2 (Sino Biological);
commercially available antibody PAT20G8AT (Prospec protein specialists);
commercially available antibody EPR11875 (abcam);
commercially available antibody MAB26371 (R&D 328002); and
commercially available antibody MAB2637 (R&D 328024).

Affinity to recombinant LAYN and thermal stability of prior art anti-LAYN antibodies was analyzed using SPR and thermal unfolding and compared to exemplary afucosylated antibodies A1, A2 and A85_afuc1 as well as to exemplary antibody moA85, A86 and P_huIgG1 (“P_huIgG1” having the variable regions of antibody P with VH as set forth in SEQ ID NO: 169 and VL as set forth in SEQ ID NO: 170 and a human IgG1 backbone having IgG1 CH1-CH2-CH3 as set forth in SEQ ID NO: 173 and IgG1 CL as set forth in SEQ ID NO: 174) described herein.

The affinities of the antibodies to LAYN-v2-His (SEQ ID NO: 190) and LAYN-v1-His (SEQ ID NO: 196) were determined on a Biacore instrument essentially as described in Example 3 with the following differences: (i) Biacore T200 instrument was used, (ii) HBS-P+ buffer was used as a running buffer and (iii) 1:3 LAYN protein dilutions from 450 nM to 5.55 nM in HBS-P+ buffer were injected. Results are shown in Table 29 below.

Thermal unfolding was measured using a Prometheus Panta (Nanotemper) by nanoDSF measurement method. This method detects changes in intrinsic Trp fluorescence as a protein unfolds during thermal treatment. Accordingly, in contrast to the dye-based DSF described in Example 4 above, nanoDSF does not require a dye. Samples were placed into high sensitivity capillaries (Cat #PR-C006, Lot051_069) in duplicates. A temperature gradient from 25 °C to 95 °C at a speed of 1 °C/min was then applied. As the protein undergoes thermal unfolding during the measurement, the fluorescence emission experiences a red shift. By tracking the intrinsic fluorescence from Trp and Tyr at 350 nm and 330 nm respectively, the thermal unfolding process can be observed. The ratio of F350 nm to F330 nm yields a unique unfolding curve for each protein. This curve is dependent on the quantity and location of the Trp and Tyr amino acid residues in the folded and unfolded protein structure. The PR.Analysis software plots the 350 nm/330 nm ratio against the temperature. Exemplified curves are shown in FIG. 29. Using this F350 nm/F330 nm ratio curve directly in the Panta software, the temperature at which unfolding begins (Tₒₙₛₑₜ) can be calculated in the Panta software. Furthermore, the inflection point (IP) of said curve can be determined from said thermal unfolding profiles (curves). Usually, the inflection point (IP) of the curve corresponds essentially to the “melting temperature” (Tm), which is the midpoint of transition, i.e. the temperature at which 50% of the protein is unfolded. Concentrations (conc.) and buffer of antibodies used in the Panta measurements (thermal unfolding) are shown in Table 28 below.

TABLE 28
Concentrations (conc.) and buffer of antibodies used in the Panta
measurements

| Name | Isotype | Conc. [mg/ml] | buffer |
|---|---|---|---|
| HL2E8_A (HL2Dir) | huIgG1 | 1.54 | Sodium Acetate Buffer + NaCl |
| HL2E8_A (HL2Dir) | moIgG1 | 1.86 | Sodium Acetate Buffer + NaCl |
| HL2E8_T | huIgG1 | 4.21 | Sodium Acetate Buffer + NaCl |
| Novus Biologicals | moIgG1 | 1 | Phosphate-buffered Saline |
| (4C11) | | | |
| Sino Biological | moIgG1 | 1 | Phosphate-buffered Saline |
| (3F7D7E2) | | | |
| HL1Std | huIgG1 | 3.49 | Phosphate-buffered Saline |
| ML1Std | huIgG1 | 0.79 | Phosphate-buffered Saline |
| EPR11875 (abcam) | rabbit IgG | 0.437 | Phosphate-buffered Saline |
| MAB26371 (R&D | moIgG2b | 1 | Phosphate-buffered Saline |
| 328002) | | | |
| moA85 | moIgG2a | 1.033 | Phosphate-buffered Saline |
| A1 | huIgG1-afuc | 1.98 | Phosphate-buffered Saline |
| A2 | huIgG1-afuc | 4.11 | Sodium Acetate Buffer + NaCl |
| A85_afuc1 | huIgG1-afuc | 2.0 | Phosphate-buffered Saline |
| A86 | huIgG1 | 4.91 | Sodium Acetate Buffer + NaCl |
| P_huIgG1 | huIgG1 | 1.24 | Phosphate-buffered Saline |

The inflection point (IP) corresponding to unfolding of the Fab (IP Fab) and the temperature where unfolding starts (Tₒₙₛₑₜ) is shown in Table 29 below. Tₒₙₛₑₜ is a parameter for overall thermodynamic stability of an antibody. In addition, the stability of the Fab portion (as indicated by the IP value of the Fab) is especially important for the potency of the antibody under stressed conditions and long-term storage. For example, the high temperature that is needed to unfold the antibodies A1 and A2 is in good agreement to the high stability detected in the RLCA assay as described in Example 3. In general, high antibody stability is an important parameter of a therapeutic antibody regarding long term storage, formulation and during the whole production process.

TABLE 29
Characterization of anti-LAYN antibodies of the present disclosure and
prior art

| | | | IP Fab [Ratio | | |
|---|---|---|---|---|---|
| | | T onset | 350 nm/330 | SPR LAYN-v1 | SPR LAYN-v2 |
| Name | Isotype | [°C] | nm in °C] | ECD KD [nM] | ECD KD [nM] |
| HL2E8_A | huIgG1 | 58.5 | 73.0 | no binding at | no binding at |
| (HL2Dir) | | | | 500 nM | 500 nM |
| HL2E8_A | moIgG1 | 53.0 | 73.8 | no binding at | no binding at |
| (HL2Dir) | | | | 500 nM | 500 nM |
| HL2E8_T | huIgG1 | 56.2 | 70.0 | no binding at | no binding at |
| | | | | 500 nM | 500 nM |
| Novus | moIgG1 | 58.7 | 74.3 | 8.3 | 17.4 |
| Biologicals | | | | | |
| (4C11) | | | | | |
| Sino | moIgG1 | 60.6 | 68.1 | 0.12 | 0.49 |
| Biological | | | | | |
| (3F7D7E2) | | | | | |
| HL1Std | huIgG1 | 62.0 | 75.4 | 799 | 834 |
| ML1Std | huIgG1 | 58.8 | 77.9 | 53 | 67 |
| PAT20G8AT | moIgG2b | nd | nd | no binding at | no binding at |
| (Prospec | | | | 500 nM | 500 nM |
| protein | | | | | |
| specialists) | | | | | |
| EPR11875 | rabbit IgG | 67.9 | 80.5 | no binding at | no binding at |
| (abcam) | | | | 500 nM | 500 nM |
| MAB26371 | moIgG2b | 60.0 | 73.4 | no binding at | 26 |
| (R&D | | | | 500 nM | |
| 328002) | | | | | |
| MAB2637 | moIgG1 | nd | nd | no binding at | no binding at |
| (R&D | | | | 500 nM | 500 nM |
| 328024) | | | | | |
| moA85 | moIgG2a | 72.5 | 85.6 | 9.9 | 17 |
| A1 | huIgG1- | 67 | 82.6 | 5.1 | 8.3 |
| | afuc | | | | |
| A2 | huIgG1- | 61.2 | 85.9 | 2.3 | 2.8 |
| | afuc | | | | |
| A85_afuc1 | huIgG1- | 66.7 | 84.2 | n.d. | n.d. |
| | afuc | | | | |
| A85_afuc2 | huIgG1- | 66.4 | 83.9 | 7.0 | 9.4 |
| | afuc | | | | |
| A86 | huIgG1 | 59.8 | 86.7 | 1.9 | 2.2 |
| P_huIgG1 | huIgG1 | 56.4 | 70.2 | 4.7 | 6.2 |

n.d.-not determined

FIG. 29 shows exemplary thermal unfolding curves measured with Prometheus Panta (Nanotemper) using the nanoDSF measurement method for antibodies moA85, A1, ML1Std, EPR11875, A85_afuc2, A86 and P_huIgG1. Ratio (350 nm/330 nm) of intrinsic fluorescence from Trp and Tyr measured at 350 nm and 330 nm respectively, is plotted against the temperature gradient. The inflection point of the thermal unfolding curve is depicted as dashed line. The start of the unfolding process (Tₒₙₛₑₜ) is depicted with a dotted line.

No specific binding to the ECD of either LAYN isoform 1 or LAYN isoform 2 could be detected for prior art antibodies HL2E8_A (HL2Dir), HL2E8_T, PAT20G8AT, EPR11875 and MAB2637 even at the highest concentration tested (500 nM). For antibody MAB26371 specific binding was only observed to the ECD of LAYN isoform 2, but not to LAYN isoform 1. Antibodies ML1Std and HL1Std showed K_{D} values of more than 50 nM. High binding affinities (K_{D} values below 50 nM) were observed for commercially available antibodies 4C11 and 3F7D7E2 as well as for all tested exemplary antibodies A1, A2, A85_afuc2, A86, moA85 and P_huIgG1 described herein.

Regarding thermal stability, exemplary antibodies moA85, A1, A2, A85_afuc1, A85_afuc2 and A86 showed the highest stability with IP Fab of 85.6, 82.6, 85.9, 84.2, 83.9 and 86.7 °C, respectively. A similarly high value was only observed for commercially available antibody EPR11875 (IP Fab: 80.47 °C), however, this antibody failed to show specific binding to the ECD of LAYN.

In summary, these data demonstrate that—in contrast to the various antibodies of the prior art tested herein—the exemplary antibodies of the present disclosure (moA85, A1, A2, A85_afuc1, A85_afuc2 and A86) not only show specific binding to the ECD of LAYN isoform 1 and 2 with high affinity, but they also have a surprisingly high stability.

These data also show that despite the differences in the sequence of the variable regions of A1, A2, A85 and A86, these antibodies show high binding affinity and thermal stability. As shown in Examples 3 and 4 above, this applies likewise for most of the other exemplary antibodies. Therefore, similarly advantages over the prior art antibodies can be reasonably expected also for other exemplary antibodies, which also show high binding affinity and thermal stability, e.g., in Examples 3 and 4.

In contrast to humanized antibodies A1-A104, antibody P was not tested in Examples 3 and 4. Interestingly, the present data show that antibody P_huIgG1 with the murine parental variable regions (and the human IgG1 backbone) has a considerably lower Fab IP of 70.2 °C as compared to humanized antibodies A1-A104 (see also Table 14 for A1-A104). These data indicate that humanization and sequence optimization surprisingly resulted in significant improvements of Fab thermal stability.

### Example 19: Specificity of Anti-LAYN Antibodies

Specificity of anti-LAYN Abs was investigated using a human plasma membrane protein cell array known as “Retrogenix® Cell Microarray” (Charles River Laboratories). This technology platform was used to screen for specific off-target binding interactions of A85 and A86. Investigation of the levels of binding of each antibody (Ab) to fixed untransfected HEK293 cells, and to cells over-expressing LAYN isoforms 1 and 2 (Uniprot #Q6UX15-1 for LAYN isoform 1 (382 amino acids) and Uniprot #Q6UX15-2 for LAYN isoform 2 (374 amino acids)) showed 5 μg/mL of each test antibody to be a suitable screening concentration. In the Library screen, a pool of A85 and A86, containing 5 μg/mL each of Ab was screened for binding against fixed human HEK293 cells, individually expressing 6105 full-length human plasma membrane proteins, secreted and cell surface-tethered human secreted proteins plus a further 400 human heterodimers. This revealed 21 library interactions altogether.

Each library interaction was re-expressed, along with 2 control receptors, and re-tested with each test antibody individually or control treatments. This was performed on both fixed and live cells.

As results, both antibodies A85 and A86 showed a significant specific interaction with the primary target, LAYN isoform 1 and 2, on both fixed and live cell microarrays. No other interactions were identified, indicating high specificity of mAbs A85 and A86 for its primary target LAYN.

In addition, cross reactivity of anti-LAYN antibodies to chondrolectin was investigated. Chondrolectin was found as the closest homologue to LAYN with 60% sequence identity. Hence, there is a theoretical possibility for cross reactivity of the anti-LAYN Abs with chondrolectin. To investigate cross reactivity, flow cytometric staining of a chondrolectin positive cell line PC3 was performed using anti-LAYN Abs A86, A1 and A2. As a positive control an anti-Chondrolectin Ab (Thermo, #MA5-24131; “aCHODL”) was used. PC3 cells were dissociated with PBS/EDTA 5 mM, 10 min at 37 °C. 200.000 cells/well (2×10\hat{ }6 cells/mL) were diluted in 100 μl of PBS and treated with 10 μg/mL of primary antibodies for 1 h on ice. The secondary Abs goat anti-Human IgG (H+L) AF488 (Thermo, cat. N. A11013; “aHu_488” or “aHu_AF488”) or goat anti-Mouse IgG (H+L) AF488 (Thermo, cat.n. A11001; “aMs_488” or “aMs_AF488”) were incubated for 30 min on ice.

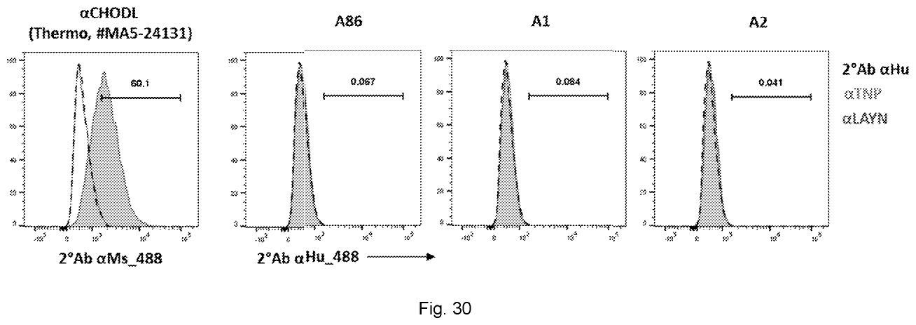

Results are shown in FIG. 30. Flow cytometry analysis showed no binding of the anti-LAYN antibodies to chondrolectin on PC3 cells at 10 μg/ml. Whereas the positive staining with the anti-Chondrolectin Ab showed specific staining of chondrolectin, confirming the presence of chondrolectin on PC3 cell used in this experiment (see FIG. 30).

In summary, these data demonstrate the specificity of the exemplary antibodies A1, A2, A85 and A86 for LAYN isoforms 1 and 2, while no binding to proteins other than LAYN was found.

### Example 20: Cross-Reactivity of Anti-LAYN Antibodies with Regard to LAYN

Cross-reactivity of an exemplary anti-LAYN antibody to LAYN orthologs expressed in different species including Macaca fascicularis, Canis lupus familiaris, Mus musculus, Oryctolagus cuniculus, Rattus norvegicus and Sus scrofa was determined by flow cytometry. Expi293F cells (Thermo Fisher Scientific) were transfected with 1 μg/mL linearized pcDNA 3.1 plasmids containing coding sequences for human LAYN v2 orthologs from cynomolgus (Macaca fascicularis, XP_005579631.3, comprising the amino acid sequence set forth in SEQ ID NO: 200), dog (Canis lupus familiaris, XP_038520860.1, comprising the amino acid sequence set forth in SEQ ID NO: 201), rat (Rattus norvegicus, XP_006243111.1, comprising the amino acid sequence set forth in SEQ ID NO: 202), mouse (Mus musculus, XP_006510392.1, comprising the amino acid sequence set forth in SEQ ID NO: 203), rabbit (Oryctolagus cuniculus, comprising the amino acid sequence set forth in SEQ ID NO: 204), or pig (Sus scrofa, NP_001231823.1, comprising the amino acid sequence set forth in SEQ ID NO: 205), fused to with a C-terminal FLAG taq (cloning performed by Genscript). 24 hours after transfection expression of LAYN v2 orthologs was checked by intracellular staining with primary mouse anti-FLAG antibody (DYKDDDDK (SEQ ID NO: 223)-Tag antibody FG4R, Invitrogen MA1-91878) and secondary Alexa Fluor 488-labeled anti-mouse IgG antibody (Alexa Fluor 488 Goat anti-Mouse IgG H+L, Invitrogen A11001). Between 39.1% (mouse) and 64.1% (pig) of transfected Expi293F cells were FLAG positive and deemed suitable for cross-reactivity assessment.

Antibody A86 was serially diluted from a starting concentration of 15 μg/mL. An appropriate AlexaFluor488-conjugated secondary antibody (Anti-Human IgG Fab specific, Sigma F5512) against the primary anti-LAYN antibody was applied, incubated for 15 min at 4 °C, and then washed off. Additionally, LIVE/DEAD™ Fixable Viability Stain 780 (BD 565388) was added to discriminate viable cells. Samples were processed on a flow cytometer and data were analyzed using FlowJo (FlowJo Software, Ashland, OR). EC50 values, for the binding of A86 anti-LAYN antibody to Expi293F cells expressing different human LAYN v2 orthologs, were calculated using GraphPad Prism (GraphPad Software, La Jolla, CA). Median Fluorescence Intensity (MFI) of Alexa Fluor 488 signal in total live cells was used for EC50 calculation.

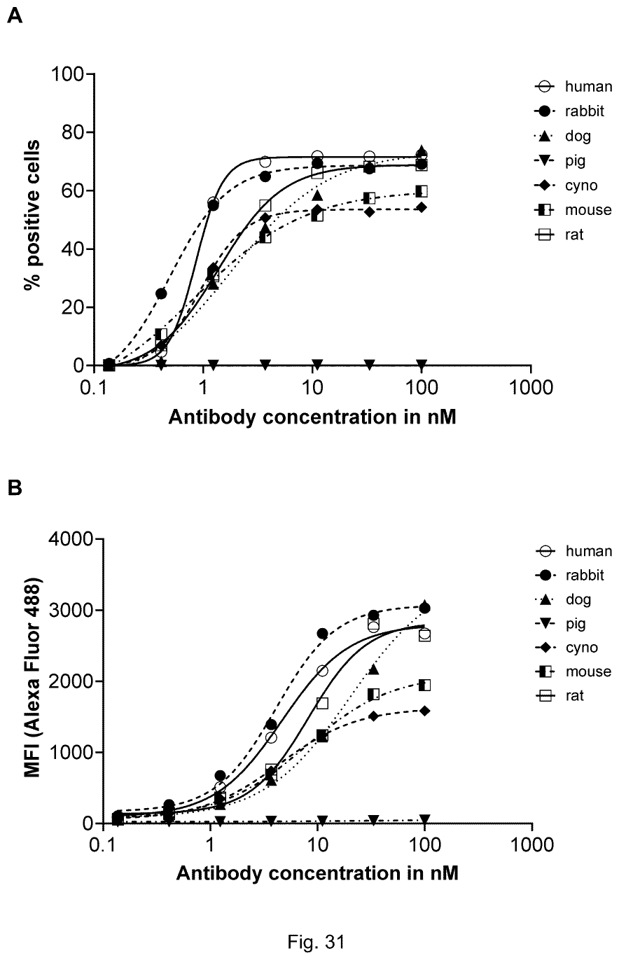

Results are shown in FIG. 31 and in Table 30 below. FIG. 31 shows the results of one representative experiment for % of AF488+ cells (A) and MFI (B).

TABLE 30
Binding of anti-LAYN antibodies to LAYN orthologs expressed in
Expi293F cells

| | EC₅₀ of % AF488+ cells | EC₅₀ of MFI (AF488) |
|---|---|---|
| Species | [nM] | [nM] |
| Human | 0.94 | 4.376 |
| Cynomolgus | 0.914 | 4.223 |
| Mouse | 0.976 | 9.136 |
| Rat | 1.21 | 7.699 |
| Rabbit | 0.633 | 3.549 |
| Dog | 1.933 | 24.13 |
| Pig | No binding | No binding |

The data demonstrate cross-reactivity of the anti-LAYN antibody A86 to the LAYN v2 ortholog in cynomolgus (cyno), mouse, rabbit and rat with similar binding to human LAYN v2. Cross reactivity to the dog ortholog of LAYN v2 is lower compared to the other orthologs, but A86 is cross reactive to dog LAYN. Only for the pig ortholog of LAYN v2 no cross reactivity was detectable.

### Example 21: Competition of Anti-LAYN Antibodies with Antibody A1

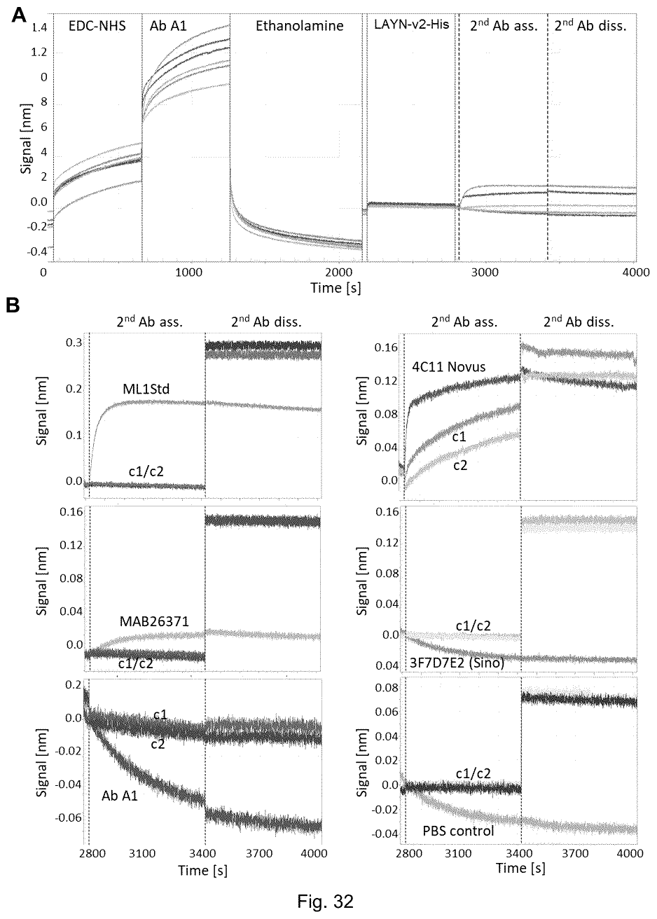

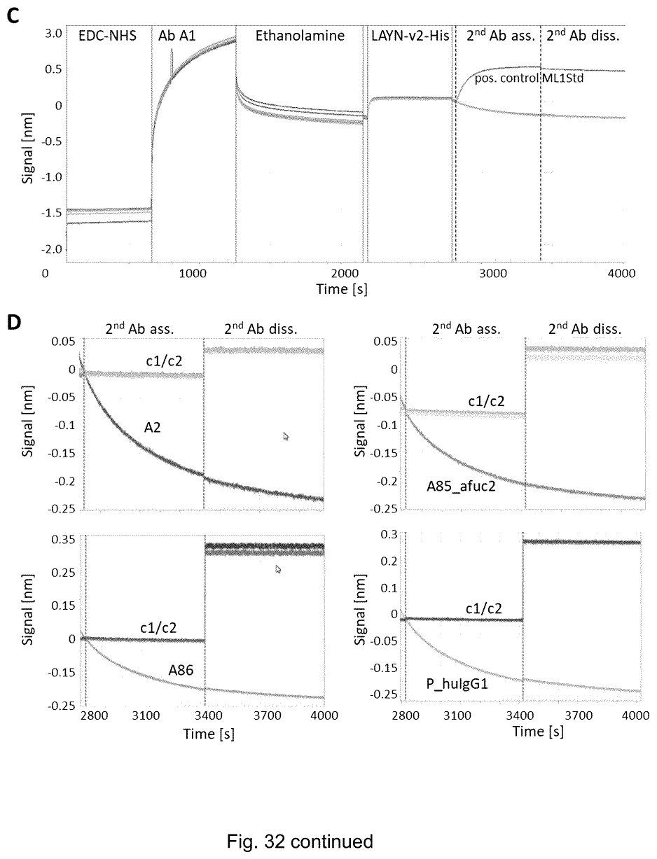

Octet competition experiments were conducted for the following anti-LAYN antibodies of the prior art (see Example 18): ML1Std, 4C11 (Novus Biologicals), MAB26371 (R&D 328002) and 3F7D7E2 (Sino Biological) and with further exemplary antibodies of the description: A2, A86, A85_afuc2 and P_huIgG1. An Octet HTX, forteBIO/pall system was used. First antibody A1 (100 μg/ml) was coupled to Amine Reactive 2nd Generation (AR2G) biosensor tips from sartorius via standard amine coupling chemistry in 10 mM sodium acetate buffer at pH 5.5. 1 M Ethanolamine pH 8.5 was used for blocking. Then, LAYN-v2-His (10 μg/ml) was bound to antibody A1 followed by binding (association and dissociation) of the respective 2^{nd} antibody. Results are shown in FIG. 32.

In two control-experiment (c1 and c2) the same setup was used, but without coupling of antibody A1 or without coupling of antibody A1 and LAYN-v2-His. In both controls no unspecific binding of LAYN-v2-His to the sensor tips was observed and only antibody 4C11 showed unspecific binding to the sensor tips in the control experiment. FIG. 32A shows the experimental setup for antibodies ML1Std, 4C11, MAB26371 (R&D 328002), and 3F7D7E2. Results for each of these antibodies (ML1Std, 4C11, MAB26371 (R&D 328002), and 3F7D7E2) are depicted in FIG. 32B, including the negative controls c1 and c2. FIG. 32C shows the experimental setup for antibodies A2, A85_afuc2, A86 and P_huIgG1. Results for each of these antibodies (A2, A85_afuc2, A86 and P_huIgG1) are depicted in FIG. 32D, including the negative controls c1 and c2.

Antibody ML1Std showed strong binding to LAYN/A1 complex indicating that ML1Std binds to a non-overlapping epitope of LAYN (i.e., to an epitope of LAYN, which does not overlap with the LAYN epitope to which antibody A1 binds). Except for the unspecific binding to the sensor tips observed for antibody 4C11 (Novus Biologicals), specific binding to LAYN/A1 complex was observed. This is indicated by the strong initial increase and curvature of the upper curve for 4C11. MAB26371 (R&D 328002) also binds to the LAYN/A1 complex. However, no binding was observed for antibody 3F7D7E2 (Sino Biological) to the LAYN/A1 complex, indicating that its LAYN binding site overlaps with that of A1. When antibody A1, A85_afuc2, A86 or P_huIgG1 were used as the 2^{nd} Antibody, no binding to LAYN/A1 complex was observed, as expected, which indicates that these antibodies bind to the same or overlapping epitopes. Also, when PBS (phosphate buffered saline) was used instead of the 2^{nd} antibody no binding to LAYN/A1 complex was observed, as expected for a control.

In summary, the data indicate that antibodies ML1Std, 4C11 and MAB26371 bind to epitopes distinct from that of antibody A1, whereas 3F7D7E2 binds to an overlapping epitope. Similarly, no binding was observed for A1, A85_afuc2, A86 and P_huIgG1, indicating that these antibodies bind to the same or overlapping epitopes.

### Example 22: Comparison of Anti-LAYN Antibodies with Regard to LAYN+ Cell Binding

The anti-LAYN antibodies of the prior art described in Example 18 were used (as commercially available or fucosylated huIgG1 versions) to compare binding to LAYN isoform 2 (SEQ ID NO: 193), LAYN isoform 1 (SEQ ID NO: 220), LAYN isoform 3 (SEQ ID NO: 221) and LAYN isoform 4 (SEQ ID NO: 222) expressing HEK293F cells. The antibodies were serially diluted from a starting concentration of 10 g/mL. The antibodies were directly labeled with Alexa Fluor 647, incubated for 15 min at 4 °C, and then washed off. Additionally LIVE/DEAD™ Fixable Aqua Dead Cell Stain was added to discriminate viable cells. Samples were processed on a flow cytometer and data was analyzed using FlowJo (FlowJo Software, Ashland, OR). EC50 values for the binding of selected anti-LAYN antibodies to LAYN isoform-expressing HEK293F cells was calculated using GraphPad Prism (GraphPad Software, La Jolla, CA). In this experiment, antibodies A1 and A2 were not afucosylated. Results are shown in Table 31 below.

TABLE 31
Functional characterization of anti-LAYN antibodies of the present disclosure and
prior art

| | | Cell binding | Cell binding | Cell binding | Cell binding |
|---|---|---|---|---|---|
| | | HEK-LAYN- | HEK-LAYN- | HEK-LAYN- | HEK-LAYN- |
| | | v1 | v2 | v3 | v4 |
| Name | Isotype | EC50 [nM] | EC50 [nM] | EC50 [nM] | EC50 [nM] |
| HL2E8 A | huIgG1 | no binding | no binding | no binding | no binding |
| (HL2Dir) | | | | | |
| HL2E8_T | huIgG1 | no binding | no binding | no binding | no binding |
| 4C11 | moIgG1 | no binding | no binding | no binding | no binding |
| 3F7D7E2 | moIgG1 | no saturation | no saturation | no binding | no binding |
| | | at 66 nM | at 66 nM | | |
| HL1Std | huIgG1 | 6.97 | no binding | no binding | no binding |
| ML1Std | huIgG1 | 3.30 | 1.48 | no binding | no binding |
| PAT20G8AT | moIgG2b | n.d. | no binding | n.d. | n.d. |
| EPR11875 | rabbit | n.d. | no binding | n.d. | n.d. |
| | IgG | | | | |
| MAB26371 | moIgG2b | no saturation | 4.23 | no binding | no binding |
| | | at 66 nM | | | |
| MAB2637 | moIgG1 | n.d. | no binding | n.d. | n.d. |
| A85 | huIgG1 | 2.54 | 1.23 | no binding | no binding |
| P_huIgG1 | huIgG1 | 8.45 | 4.18 | no binding | no binding |
| A86 | huIgG1 | 5.86 | 1.09 | no binding | no binding |
| A1 | huIgG1 | 2.41 | 1.58 | no binding | no binding |
| A2 | huIgG1 | 3.8 | 2.04 | no binding | no binding |

(n.d. not determined)

In alignment with the SPR results presented in Example 18, no specific binding to HEK cells expressing LAYN isoform 2 could be detected for prior art antibodies HL2E8_A (HL2Dir), HL2E8_T, PAT20G8AT, EPR11875 and MAB2637 even at the highest concentration tested (66 nM). For antibody 3F7D7E2 only weak binding to LAYN isoform 2 was detectable at the highest tested concentration. Additionally, no binding was observed to LAYN isoform 2 for 4C11 and HL1Std, despite detectable binding in SPR as shown in Example 18. Only for the prior art antibodies ML1Std and MAB26371 binding to cellular LAYN isoform 2 was detectable with an EC50 of 1.48 nM and 4.23 nM, respectively. Binding of prior art antibodies to LAYN isoform 1 expressed on HEK293F cells was only observed for HL1Std and ML1Std with an EC50 of 6.97 nM and 3.30 nM, respectively. For antibody 3F7D7E2 only weak binding was observed to LAYN isoform 1 at the highest tested concentration. Furthermore, none of the tested prior art antibodies showed detectable binding to cells expressing LAYN isoform 3 or 4. Out of all prior art antibodies tested, only ML1Std clearly demonstrated binding to both, LAYN isoform 1 or 2 expressing cells. The exemplary antibodies A85 and A1 described herein demonstrated the highest affinity among the antibodies tested for binding to HEK cells expressing LAYN isoform 1 (EC50 of 2.54 and 2.41 nM, respectively). The exemplary antibodies A85 and A86 described herein demonstrated the highest affinity among the antibodies tested for binding to HEK cells expressing LAYN isoform 2 (EC50 of 1.23 and 1.09 nM, respectively).

### Example 23: Comparison of ADCC Activity of Anti-LAYN Antibodies

The anti-LAYN antibodies HL2E8_A, HL2E8_T, HL1Std and ML1Std of the prior art described in Example 18 were produced with afucosylated human IgG1 (also referred to herein as “huIgG1” or “hIgG1”) isotype and were used to assess ADCC potential. Jurkat cells expressing full length LAYN isoform 2 (SEQ ID NO: 193) were co-cultured in a 1:1 ratio with CD16AV158-expressing Jurkat NFAT luciferase reporter cells (Promega, Madison, WI) at 37 °C for 4 h in the presence of an anti-LAYN antibody or a control antibody. BIO-GLO™ (Promega, Madison, WI) was added to the cells and then the luminescence was read for measurement of luciferase activity. The half-maximal effective concentration (EC50) was calculated using GraphPad Prism. Results are shown in Table 32 below.

TABLE 32
Functional characterization of anti-LAYN antibodies
of the present disclosure and prior art

| | | ADCC reporter assay |
|---|---|---|
| | | CD16A V |
| Name | Isotype | EC50 [nM] |
| HL2E8_A | huIgG1 afuc | no ADCC |
| (HL2Dir) | | |
| HL2E8_T | huIgG1 afuc | no ADCC |
| HL1Std | huIgG1 afuc | no ADCC |
| ML1Std | huIgG1 afuc | 0.151 |
| P_huIgG1 | huIgG1 afuc | 0.018 |
| A85_afuc2 | huIgG1 afuc | 0.020 |
| A86_afuc | huIgG1 afuc | 0.010 |
| A1 | huIgG1 afuc | 0.007 |
| A2 | huIgG1 afuc | 0.007 |

Among the tested prior art antibodies, HL2E8_A (HL2Dir), HL2E8_T and HL1Std demonstrated no ADCC potential, only ML1Std was capable of CD16A engagement upon binding to Jurkat cells expressing LAYN and induced ADCC with an EC50 value of 0.151 nM. The lack of ADCC of prior art antibodies HL2E8_A (HL2Dir), HL2E8_T and HL1Std is in line with their lack of binding to cells expressing LAYN isoform 2 (LAYN v2), as shown in Example 22 (Table 31).

In contrast, all of the tested anti-LAYN antibodies of the present disclosure (P_huIgG1, A85, A86, A1 and A2) were capable of CD16A engagement upon binding to Jurkat cells expressing LAYN, and induced ADCC with EC50 values of ≤0.02 nM.

In summary, a 7.5- to 20-fold higher ADCC potency was surprisingly observed for all tested exemplary antibodies of the present disclosure (P_huIgG1, A85, A86, A1 and A2) compared to prior art antibody ML1Std, even though the same backbone (afucosylated human IgG1) was used for all antibodies tested and a comparable affinity to LAYN isoform 2 was observed in Example 22. This indicates the superior capability of the antibodies of the present disclosure to induce ADCC on LAYN-expressing cells, which is assumed to be the mechanism of action underlying antibody-mediated depletion of intratumoral regulatory T cells (Treg depleting antibodies). Without being bound to any theory, it is assumed that the large differences regarding ADCC induction of the antibodies of the present disclosure vs. ML1Std may be explained by the different LAYN epitopes (see Example 21).

Table of sequences and SEQ ID NOs (Sequence listing)

| SEQ | | |
|---|---|---|
| ID | | |
| NO | Sequence | Comment |
| 1 | SYWMD | HCDR1 A1- |
| | | A50, A52-A102, |
| | | P |
| 2 | NIYPSSGTTNYAQKFQG | HCDR2 A1, A3, |
| | | A57-A64 |
| 3 | ERYGRNAAEY | HCDR3 A1-A9, |
| | | A30, A57-A81 |
| 4 | RASQGIGSWLA | LCDR1 A1, A3, |
| | | A5, A8, A10, |
| | | A54, A66, A71, |
| | | A78 |
| 5 | AATSLAD | LCDR2 A1-A15, |
| | | A20-A88, A92- |
| | | A94, A98-A104, |
| | | P |
| 6 | QQLYSTPLT | LCDR3 A1-A11, |
| | | A15-A97, A99- |
| | | A104, P |
| 7 | QVQLVQSGAEVKKPGASVKVSCKASGYTFTSYWMDWVRQAPGQGLEWMG | VH A1, A61- |
| | NIYPSSGTTNYAQKFQGRVTMTVDKSTSTAYMELSSLRSEDTAVYYCARERYGR | A64 |
| | NAAEYWGQGTLVTVSS | |
| 8 | DIQMTQSPSSVSASVGDRVTITCRASQGIGSWLAWYQQKPGKAPKLLIYAATSL | VL A1, A3, A5, |
| | ADGVPSRFSGSGSGTDFTLTISSLQPEDFATYYCQQLYSTPLTFGGGTKLEIK | A8, A10, A54, |
| | | A66, A71, A78 |
| 9 | QVQLVQSGAEVKKPGASVKVSCKASGYTFTSYWMDWVRQAPGQGLEWMG | HC A1, A61- |
| | NIYPSSGTTNYAQKFQGRVTMTVDKSTSTAYMELSSLRSEDTAVYYCARERYGR | A64 |
| | NAAEYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 10 | DIQMTQSPSSVSASVGDRVTITCRASQGIGSWLAWYQQKPGKAPKLLIYAATSL | LC A1, A3, A5, |
| | ADGVPSRFSGSGSGTDFTLTISSLQPEDFATYYCQQLYSTPLTFGGGTKLEIKRTV | A8, A10, A54, |
| | AAPSVFIFPPSDEQLKSGTASVVCLLNNFYPREAKVQWKVDNALQSGNSQESV | A66, A71, A78 |
| | TEQDSKDSTYSLSSTLTLSKADYEKHKVYACEVTHQGLSSPVTKSFNRGEC | |
| 11 | NIYPGSGTTNYAQKFQG | HCDR2 A2, A7, |
| | | A48, A77-A79 |
| 12 | RASQTIGTWLA | LCDR1 A2, A4, |
| | | A6, A9, A11, |
| | | A26, A55, A56, |
| | | A58, A59, A62, |
| | | A63, A67, A68, |
| | | A72, A75, A79, |
| | | A80, A83 |
| 13 | QVQLVQSGSEVKKPGASVKVSCKASGYTFTSYWMDWVRQAPGQGLEWMG | VH A2, A7, |
| | NIYPGSGTTNYAQKFQGRATMTVDKSTSTAYMELSSLRSEDTAVYYCARERYGR | A77-A79 |
| | NAAEYWGQGTLVTVSS | |
| 14 | DIQMTQSPSSVSASVGDRVTITCRASQTIGTWLAWYQQKPGKAPKLLIYAATSL | VL A2, A4, A6, |
| | ADGVPSRFSGSGSGTDFTLTISSLQPEDFATYYCQQLYSTPLTFGGGTKLEIK | A9, A11, A55, |
| | | A58, A62, A67 |
| 15 | QVQLVQSGSEVKKPGASVKVSCKASGYTFTSYWMDWVRQAPGQGLEWMG | HC A2, A7, |
| | NIYPGSGTTNYAQKFQGRATMTVDKSTSTAYMELSSLRSEDTAVYYCARERYGR | A77-A79 |
| | NAAEYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 16 | DIQMTQSPSSVSASVGDRVTITCRASQTIGTWLAWYQQKPGKAPKLLIYAATSL | LC A2, A4, A6, |
| | ADGVPSRFSGSGSGTDFTLTISSLQPEDFATYYCQQLYSTPLTFGGGTKLEIKRTV | A9, A11, A55, |
| | AAPSVFIFPPSDEQLKSGTASVVCLLNNFYPREAKVQWKVDNALQSGNSQESV | A58, A62, A67 |
| | TEQDSKDSTYSLSSTLTLSKADYEKHKVYACEVTHQGLSSPVTKSFNRGEC | |
| 17 | QVQLVQSGAEVKKPGASVKVSCKASGYTFTSYWMDWVRQAPGQGLEWMG | VH A3, A57- |
| | NIYPSSGTTNYAQKFQGRVTMTVDTSTSTAYMELSSLRSEDTAVYYCARERYGR | A60 |
| | NAAEYWGQGTLVTVSS | |
| 18 | QVQLVQSGAEVKKPGASVKVSCKASGYTFTSYWMDWVRQAPGQGLEWMG | HC A3, A57- |
| | NIYPSSGTTNYAQKFQGRVTMTVDTSTSTAYMELSSLRSEDTAVYYCARERYGR | A60 |
| | NAAEYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 19 | NIYPGSGSTNYAQKFQG | HCDR2 A4-A6, |
| | | A8-A47, A51- |
| | | A56, A70-A76, |
| | | A80-A100, |
| | | A103, A104 |
| 20 | QVQLVQSGSEVKKPGASVKVSCKASGYTFTSYWMDWVRQAPGQGLEWMG | VH A4, A70- |
| | NIYPGSGSTNYAQKFQGRATLTVDKSTSTAYMELSSLRSEDTAVYYCARERYGRN | A73 |
| | AAEYWGQGTLVTVSS | |
| 21 | QVQLVQSGSEVKKPGASVKVSCKASGYTFTSYWMDWVRQAPGQGLEWMG | HC A4, A70- |
| | NIYPGSGSTNYAQKFQGRATLTVDKSTSTAYMELSSLRSEDTAVYYCARERYGRN | A73 |
| | AAEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 22 | QVQLVQSGSEVKKPGASVKVSCKASGYTFTSYWMDWVRQAPGQGLEWMG | VH A5, A6, |
| | NIYPGSGSTNYAQKFQGRATMTVDKSTSTAYMELSSLRSEDTAVYYCARERYGR | A74-A76 |
| | NAAEYWGQGTLVTVSS | |
| 23 | QVQLVQSGSEVKKPGASVKVSCKASGYTFTSYWMDWVRQAPGQGLEWMG | HC A5, A6, |
| | NIYPGSGSTNYAQKFQGRATMTVDKSTSTAYMELSSLRSEDTAVYYCARERYGR | A74-A76 |
| | NAAEYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 24 | LASQTIGTWLA | LCDR1 A7, |
| | | A12-A20, A27- |
| | | A53, A57, A60, |
| | | A61, A64, A65, |
| | | A69, A70, A73, |
| | | A74, A76, A77, |
| | | A81, A82, A84- |
| | | A104, P |
| 25 | DIQMTQSPSSVSASVGDRVTITCLASQTIGTWLAWYQQKPGKSPKLLIYAATSL | VL A7, A15, |
| | ADGVPSRFSGSGSGTDFTLTISSLQPEDFATYYCQQLYSTPLTFGGGTKLEIK | A60, A64, A69, |
| | | A73, A76, A81, |
| | | A84 |
| 26 | DIQMTQSPSSVSASVGDRVTITCLASQTIGTWLAWYQQKPGKSPKLLIYAATSL | LC A7, A15, |
| | ADGVPSRFSGSGSGTDFTLTISSLQPEDFATYYCQQLYSTPLTFGGGTKLEIKRTV | A60, A64, A69, |
| | AAPSVFIFPPSDEQLKSGTASVVCLLNNFYPREAKVQWKVDNALQSGNSQESV | A73, A76, A81, |
| | TEQDSKDSTYSLSSTLTLSKADYEKHKVYACEVTHQGLSSPVTKSFNRGEC | A84 |
| 27 | QVQLVQSGSEVKKPGASVKVSCKASGYTFTSYWMDWVRQAPGQGLEWMG | VH A8, A9, A30, |
| | NIYPGSGSTNYAQKFQGRATLTVDKSTSTAYMELSSLRSEDTAVYYCTRERYGRN | A80, A81 |
| | AAEYWGQGTLVTVSS | |
| 28 | QVQLVQSGSEVKKPGASVKVSCKASGYTFTSYWMDWVRQAPGQGLEWMG | HC A8, A9, |
| | NIYPGSGSTNYAQKFQGRATLTVDKSTSTAYMELSSLRSEDTAVYYCTRERYGRN | A30, A80, A81 |
| | AAEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 29 | EKYGRNAMEY | HCDR3 A10, |
| | | A11, A82-A84 |
| 30 | QVQLVQSGSEVKKPGASVKVSCKASGYTFTSYWMDWVRQAPGQGLEWMG | VH A10, A11, |
| | NIYPGSGSTNYAQKFQGRATLTVDKSTSTAYMELSSLRSEDTAVYYCTREKYGRN | A82-A84 |
| | AMEYWGQGTLVTVSS | |
| 31 | QVQLVQSGSEVKKPGASVKVSCKASGYTFTSYWMDWVRQAPGQGLEWMG | HC A10, A11, |
| | NIYPGSGSTNYAQKFQGRATLTVDKSTSTAYMELSSLRSEDTAVYYCTREKYGRN | A82-A84 |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 32 | ERYGRNAMEY | HCDR3 A12- |
| | | A27, A35-A56, |
| | | A86, A98, |
| | | A101-A104 |
| 33 | QQLYSFPLT | LCDR3 A12 |
| 34 | QVQLVQSGSEVKKPGASVKVSCKASGYTFTSYWMDWVRQAPGQGLEWMG | VH A12-A27, |
| | NIYPGSGSTNYAQKFQGRATLTVDKSTSTAYMELSSLRSEDTAVYYCTRERYGRN | A54-A56, A86, |
| | AMEYWGQGTLVTVSS | A98 |
| 35 | DIQMTQSPSSVSASVGDRVTITCLASQTIGTWLAWYQQKPGKSPKLLIYAATSL | VL A12 |
| | ADGVPSRFSGSGSGTDFTFTISSLQPEDFATYYCQQLYSFPLTFGGGTKLEIK | |
| 36 | QVQLVQSGSEVKKPGASVKVSCKASGYTFTSYWMDWVRQAPGQGLEWMG | HC A12-A27, |
| | NIYPGSGSTNYAQKFQGRATLTVDKSTSTAYMELSSLRSEDTAVYYCTRERYGRN | A54-A56, A86, |
| | AMEYWGQGTLVTVSSASTKGPSVFPLAPSSKSTSGGTAALGCLVKDYFPEPVTV | A98 |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 37 | DIQMTQSPSSVSASVGDRVTITCLASQTIGTWLAWYQQKPGKSPKLLIYAATSL | LC A12 |
| | ADGVPSRFSGSGSGTDFTFTISSLQPEDFATYYCQQLYSFPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 38 | QQAYSTPLT | LCDR3 A13, |
| 39 | DIQMTQSPSSVSASVGDRVTITCLASQTIGTWLAWYQQKPGKSPKLLIYAATSL | VL A13 |
| | ADGVPSRFSGSGSGTDFTFTISSLQPEDFATYYCQQAYSTPLTFGGGTKLEIK | |
| 40 | DIQMTQSPSSVSASVGDRVTITCLASQTIGTWLAWYQQKPGKSPKLLIYAATSL | LC A13 |
| | ADGVPSRFSGSGSGTDFTFTISSLQPEDFATYYCQQAYSTPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 41 | QQSYSTPLT | LCDR3 A14 |
| 42 | DIQMTQSPSSVSASVGDRVTITCLASQTIGTWLAWYQQKPGKSPKLLIYAATSL | VLA14 |
| | ADGVPSRFSGSGSGTDFTFTISSLQPEDFATYYCQQSYSTPLTFGGGTKLEIK | |
| 43 | DIQMTQSPSSVSASVGDRVTITCLASQTIGTWLAWYQQKPGKSPKLLIYAATSL | LC A14 |
| | ADGVPSRFSGSGSGTDFTFTISSLQPEDFATYYCQQSYSTPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 44 | AATSLAT | LCDR2 A16 |
| 45 | DIQMTQSPSSVSASVGDRVTITCLASQTIGTWLAWYQQKPGKSPKLLIYAATSL | VLA16 |
| | ATGVPSRFSGSGSGTDFTFTISSLQPEDFATYYCQQLYSTPLTFGGGTKLEIK | |
| 46 | DIQMTQSPSSVSASVGDRVTITCLASQTIGTWLAWYQQKPGKSPKLLIYAATSL | LC A16 |
| | ATGVPSRFSGSGSGTDFTFTISSLQPEDFATYYCQQLYSTPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 47 | AATSLAS | LCDR2 A17 |
| 48 | DIQMTQSPSSVSASVGDRVTITCLASQTIGTWLAWYQQKPGKSPKLLIYAATSL | VL A17 |
| | ASGVPSRFSGSGSGTDFTFTISSLQPEDFATYYCQQLYSTPLTFGGGTKLEIK | |
| 49 | DIQMTQSPSSVSASVGDRVTITCLASQTIGTWLAWYQQKPGKSPKLLIYAATSL | LC A17 |
| | ASGVPSRFSGSGSGTDFTFTISSLQPEDFATYYCQQLYSTPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 50 | AATSLQD | LCDR2 A18 |
| 51 | DIQMTQSPSSVSASVGDRVTITCLASQTIGTWLAWYQQKPGKSPKLLIYAATSL | VL A18 |
| | QDGVPSRFSGSGSGTDFTFTISSLQPEDFATYYCQQLYSTPLTFGGGTKLEIK | |
| 52 | DIQMTQSPSSVSASVGDRVTITCLASQTIGTWLAWYQQKPGKSPKLLIYAATSL | LC A18 |
| | QDGVPSRFSGSGSGTDFTFTISSLQPEDFATYYCQQLYSTPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 53 | AASSLAD | LCDR2 A19 |
| 54 | DIQMTQSPSSVSASVGDRVTITCLASQTIGTWLAWYQQKPGKSPKLLIYAASSL | VL A19 |
| | ADGVPSRFSGSGSGTDFTFTISSLQPEDFATYYCQQLYSTPLTFGGGTKLEIK | |
| 55 | DIQMTQSPSSVSASVGDRVTITCLASQTIGTWLAWYQQKPGKSPKLLIYAASSL | LC A19 |
| | ADGVPSRFSGSGSGTDFTFTISSLQPEDFATYYCQQLYSTPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 56 | DIQMTQSPSSVSASVGDRVTITCLASQTIGTWLAWYQQKPGKAPKLLIYAATSL | VL A20 |
| | ADGVPSRFSGSGSGTDFTFTISSLQPEDFATYYCQQLYSTPLTFGGGTKLEIK | |
| 57 | DIQMTQSPSSVSASVGDRVTITCLASQTIGTWLAWYQQKPGKAPKLLIYAATSL | LC A20 |
| | ADGVPSRFSGSGSGTDFTFTISSLQPEDFATYYCQQLYSTPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 58 | LASQTIGTYLA | LCDR1 A21 |
| 59 | DIQMTQSPSSVSASVGDRVTITCLASQTIGTYLAWYQQKPGKSPKLLIYAATSLA | VL A21 |
| | DGVPSRFSGSGSGTDFTFTISSLQPEDFATYYCQQLYSTPLTFGGGTKLEIK | |
| 60 | DIQMTQSPSSVSASVGDRVTITCLASQTIGTYLAWYQQKPGKSPKLLIYAATSLA | LC A21 |
| | DGVPSRFSGSGSGTDFTFTISSLQPEDFATYYCQQLYSTPLTFGGGTKLEIKRTVA | |
| | APSVFIFPPSDEQLKSGTASVVCLLNNFYPREAKVQWKVDNALQSGNSQESVT | |
| | EQDSKDSTYSLSSTLTLSKADYEKHKVYACEVTHQGLSSPVTKSFNRGEC | |
| 61 | LASQTIGSWLA | LCDR1 A22 |
| 62 | DIQMTQSPSSVSASVGDRVTITCLASQTIGSWLAWYQQKPGKSPKLLIYAATSL | VL A22 |
| | ADGVPSRFSGSGSGTDFTFTISSLQPEDFATYYCQQLYSTPLTFGGGTKLEIK | |
| 63 | DIQMTQSPSSVSASVGDRVTITCLASQTIGSWLAWYQQKPGKSPKLLIYAATSL | LC A22 |
| | ADGVPSRFSGSGSGTDFTFTISSLQPEDFATYYCQQLYSTPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 64 | LASQTISTWLA | LCDR1 A23 |
| 65 | DIQMTQSPSSVSASVGDRVTITCLASQTISTWLAWYQQKPGKSPKLLIYAATSLA | VL A23 |
| | DGVPSRFSGSGSGTDFTFTISSLQPEDFATYYCQQLYSTPLTFGGGTKLEIK | |
| 66 | DIQMTQSPSSVSASVGDRVTITCLASQTISTWLAWYQQKPGKSPKLLIYAATSLA | LC A23 |
| | DGVPSRFSGSGSGTDFTFTISSLQPEDFATYYCQQLYSTPLTFGGGTKLEIKRTVA | |
| | APSVFIFPPSDEQLKSGTASVVCLLNNFYPREAKVQWKVDNALQSGNSQESVT | |
| | EQDSKDSTYSLSSTLTLSKADYEKHKVYACEVTHQGLSSPVTKSFNRGEC | |
| 67 | LASQGIGTWLA | LCDR1 A24 |
| 68 | DIQMTQSPSSVSASVGDRVTITCLASQGIGTWLAWYQQKPGKSPKLLIYAATSL | VL A24 |
| | ADGVPSRFSGSGSGTDFTFTISSLQPEDFATYYCQQLYSTPLTFGGGTKLEIK | |
| 69 | DIQMTQSPSSVSASVGDRVTITCLASQGIGTWLAWYQQKPGKSPKLLIYAATSL | LC A24 |
| | ADGVPSRFSGSGSGTDFTFTISSLQPEDFATYYCQQLYSTPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 70 | LASQSIGTWLA | LCDR1 A25 |
| 71 | DIQMTQSPSSVSASVGDRVTITCLASQSIGTWLAWYQQKPGKSPKLLIYAATSL | VL A25 |
| | ADGVPSRFSGSGSGTDFTFTISSLQPEDFATYYCQQLYSTPLTFGGGTKLEIK | |
| 72 | DIQMTQSPSSVSASVGDRVTITCLASQSIGTWLAWYQQKPGKSPKLLIYAATSL | LC A25 |
| | ADGVPSRFSGSGSGTDFTFTISSLQPEDFATYYCQQLYSTPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 73 | DIQMTQSPSSVSASVGDRVTITCRASQTIGTWLAWYQQKPGKSPKLLIYAATSL | VL A26 |
| | ADGVPSRFSGSGSGTDFTFTISSLQPEDFATYYCQQLYSTPLTFGGGTKLEIK | |
| 74 | DIQMTQSPSSVSASVGDRVTITCRASQTIGTWLAWYQQKPGKSPKLLIYAATSL | LC A26 |
| | ADGVPSRFSGSGSGTDFTFTISSLQPEDFATYYCQQLYSTPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 75 | DIQMTQSPSSLSASVGDRVTITCLASQTIGTWLAWYQQKPGKSPKLLIYAATSL | VL A27 |
| | ADGVPSRFSGSGSGTDFTFTISSLQPEDFATYYCQQLYSTPLTFGGGTKLEIK | |
| 76 | DIQMTQSPSSLSASVGDRVTITCLASQTIGTWLAWYQQKPGKSPKLLIYAATSL | LC A27 |
| | ADGVPSRFSGSGSGTDFTFTISSLQPEDFATYYCQQLYSTPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 77 | ERYGRNAMEA | HCDR3 A28 |
| 78 | QVQLVQSGSEVKKPGASVKVSCKASGYTFTSYWMDWVRQAPGQGLEWMG | VH A28 |
| | NIYPGSGSTNYAQKFQGRATLTVDKSTSTAYMELSSLRSEDTAVYYCTRERYGRN | |
| | AMEAWGQGTLVTVSS | |
| 79 | DIQMTQSPSSVSASVGDRVTITCLASQTIGTWLAWYQQKPGKSPKLLIYAATSL | VL A28-A53, |
| | ADGVPSRFSGSGSGTDFTFTISSLQPEDFATYYCQQLYSTPLTFGGGTKLEIK | A57, A61, A65, |
| | | A70, A74, A77, |
| | | A82, A85-A88, |
| | | A92-A94, A99- |
| | | A104 |
| 80 | QVQLVQSGSEVKKPGASVKVSCKASGYTFTSYWMDWVRQAPGQGLEWMG | HC A28 |
| | NIYPGSGSTNYAQKFQGRATLTVDKSTSTAYMELSSLRSEDTAVYYCTRERYGRN | |
| | AMEAWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 81 | DIQMTQSPSSVSASVGDRVTITCLASQTIGTWLAWYQQKPGKSPKLLIYAATSL | LC A28-A53, |
| | ADGVPSRFSGSGSGTDFTFTISSLQPEDFATYYCQQLYSTPLTFGGGTKLEIKRTV | A57, A61, A65, |
| | AAPSVFIFPPSDEQLKSGTASVVCLLNNFYPREAKVQWKVDNALQSGNSQESV | A70, A74, A77, |
| | TEQDSKDSTYSLSSTLTLSKADYEKHKVYACEVTHQGLSSPVTKSFNRGEC | A82, A85-A88, |
| | | A92-A94, A99- |
| | | A104 |
| 82 | ERYGRNAMAY | HCDR3 A29 |
| 83 | QVQLVQSGSEVKKPGASVKVSCKASGYTFTSYWMDWVRQAPGQGLEWMG | VH A29 |
| | NIYPGSGSTNYAQKFQGRATLTVDKSTSTAYMELSSLRSEDTAVYYCTRERYGRN | |
| | AMAYWGQGTLVTVSS | |
| 84 | QVQLVQSGSEVKKPGASVKVSCKASGYTFTSYWMDWVRQAPGQGLEWMG | HC A29 |
| | NIYPGSGSTNYAQKFQGRATLTVDKSTSTAYMELSSLRSEDTAVYYCTRERYGRN | |
| | AMAYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 85 | ERYGRAAMEY | HCDR3 A31 |
| 86 | QVQLVQSGSEVKKPGASVKVSCKASGYTFTSYWMDWVRQAPGQGLEWMG | VH A31 |
| | NIYPGSGSTNYAQKFQGRATLTVDKSTSTAYMELSSLRSEDTAVYYCTRERYGRA | |
| | AMEYWGQGTLVTVSS | |
| 87 | QVQLVQSGSEVKKPGASVKVSCKASGYTFTSYWMDWVRQAPGQGLEWMG | HC A31 |
| | NIYPGSGSTNYAQKFQGRATLTVDKSTSTAYMELSSLRSEDTAVYYCTRERYGRA | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 88 | ERYARNAMEY | HCDR3 A32 |
| 89 | QVQLVQSGSEVKKPGASVKVSCKASGYTFTSYWMDWVRQAPGQGLEWMG | VH A32 |
| | NIYPGSGSTNYAQKFQGRATLTVDKSTSTAYMELSSLRSEDTAVYYCTRERYARN | |
| | AMEYWGQGTLVTVSS | |
| 90 | QVQLVQSGSEVKKPGASVKVSCKASGYTFTSYWMDWVRQAPGQGLEWMG | HC A32 |
| | NIYPGSGSTNYAQKFQGRATLTVDKSTSTAYMELSSLRSEDTAVYYCTRERYARN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 91 | EAYGRNAMEY | HCDR3 A33 |
| 92 | QVQLVQSGSEVKKPGASVKVSCKASGYTFTSYWMDWVRQAPGQGLEWMG | VH A33 |
| | NIYPGSGSTNYAQKFQGRATLTVDKSTSTAYMELSSLRSEDTAVYYCTREAYGRN | |
| | AMEYWGQGTLVTVSS | |
| 93 | QVQLVQSGSEVKKPGASVKVSCKASGYTFTSYWMDWVRQAPGQGLEWMG | HC A33 |
| | NIYPGSGSTNYAQKFQGRATLTVDKSTSTAYMELSSLRSEDTAVYYCTREAYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 94 | ARYGRNAMEY | HCDR3 A34 |
| 95 | QVQLVQSGSEVKKPGASVKVSCKASGYTFTSYWMDWVRQAPGQGLEWMG | VH A34 |
| | NIYPGSGSTNYAQKFQGRATLTVDKSTSTAYMELSSLRSEDTAVYYCTRARYGRN | |
| | AMEYWGQGTLVTVSS | |
| 96 | QVQLVQSGSEVKKPGASVKVSCKASGYTFTSYWMDWVRQAPGQGLEWMG | HC A34 |
| | NIYPGSGSTNYAQKFQGRATLTVDKSTSTAYMELSSLRSEDTAVYYCTRARYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 97 | QVQLVQSGSEVKKPGASVKVSCKASGYTFTSYWMDWVRQAPGQGLEWMG | VH A35 |
| | NIYPGSGSTNYAQKFQGRATLTVDKSTSTAYMELSSLRSEDTAVYYCARERYGRN | |
| | AMEYWGQGTLVTVSS | |
| 98 | QVQLVQSGSEVKKPGASVKVSCKASGYTFTSYWMDWVRQAPGQGLEWMG | HC A35 |
| | NIYPGSGSTNYAQKFQGRATLTVDKSTSTAYMELSSLRSEDTAVYYCARERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 99 | QVQLVQSGSEVKKPGASVKVSCKASGYTFTSYWMDWVRQAPGQGLEWMG | VH A36 |
| | NIYPGSGSTNYAQKFQGRATLTVDKSTSTAYMELSSLRSDDTAVYYCTRERYGRN | |
| | AMEYWGQGTLVTVSS | |
| 100 | QVQLVQSGSEVKKPGASVKVSCKASGYTFTSYWMDWVRQAPGQGLEWMG | HC A36 |
| | NIYPGSGSTNYAQKFQGRATLTVDKSTSTAYMELSSLRSDDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 101 | QVQLVQSGSEVKKPGASVKVSCKASGYTFTSYWMDWVRQAPGQGLEWMG | VH A37 |
| | NIYPGSGSTNYAQKFQGRATLTVDKSTSTAYMELSRLRSEDTAVYYCTRERYGRN | |
| | AMEYWGQGTLVTVSS | |
| 102 | QVQLVQSGSEVKKPGASVKVSCKASGYTFTSYWMDWVRQAPGQGLEWMG | HC A37 |
| | NIYPGSGSTNYAQKFQGRATLTVDKSTSTAYMELSRLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 103 | QVQLVQSGSEVKKPGASVKVSCKASGYTFTSYWMDWVRQAPGQGLEWMG | VH A38 |
| | NIYPGSGSTNYAQKFQGRATLTVDKSTSTLYMELSSLRSEDTAVYYCTRERYGRN | |
| | AMEYWGQGTLVTVSS | |
| 104 | QVQLVQSGSEVKKPGASVKVSCKASGYTFTSYWMDWVRQAPGQGLEWMG | HC A38 |
| | NIYPGSGSTNYAQKFQGRATLTVDKSTSTLYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 105 | QVQLVQSGSEVKKPGASVKVSCKASGYTFTSYWMDWVRQAPGQGLEWMG | VH A39 |
| | NIYPGSGSTNYAQKFQGRATLTVDKSTSTVYMELSSLRSEDTAVYYCTRERYGRN | |
| | AMEYWGQGTLVTVSS | |
| 106 | QVQLVQSGSEVKKPGASVKVSCKASGYTFTSYWMDWVRQAPGQGLEWMG | HC A39 |
| | NIYPGSGSTNYAQKFQGRATLTVDKSTSTVYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 107 | QVQLVQSGSEVKKPGASVKVSCKASGYTFTSYWMDWVRQAPGQGLEWMG | VH A40 |
| | NIYPGSGSTNYAQKFQGRATLTVDKSKSTAYMELSSLRSEDTAVYYCTRERYGRN | |
| | AMEYWGQGTLVTVSS | |
| 108 | QVQLVQSGSEVKKPGASVKVSCKASGYTFTSYWMDWVRQAPGQGLEWMG | HC A40 |
| | NIYPGSGSTNYAQKFQGRATLTVDKSKSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 109 | QVQLVQSGSEVKKPGASVKVSCKASGYTFTSYWMDWVRQAPGQGLEWMG | VH A41 |
| | NIYPGSGSTNYAQKFQGRATLTVDKSISTAYMELSSLRSEDTAVYYCTRERYGRN | |
| | AMEYWGQGTLVTVSS | |
| 110 | QVQLVQSGSEVKKPGASVKVSCKASGYTFTSYWMDWVRQAPGQGLEWMG | HC A41 |
| | NIYPGSGSTNYAQKFQGRATLTVDKSISTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 111 | QVQLVQSGSEVKKPGASVKVSCKASGYTFTSYWMDWVRQAPGQGLEWMG | VH A42 |
| | NIYPGSGSTNYAQKFQGRATLTVDTSTSTAYMELSSLRSEDTAVYYCTRERYGRN | |
| | AMEYWGQGTLVTVSS | |
| 112 | QVQLVQSGSEVKKPGASVKVSCKASGYTFTSYWMDWVRQAPGQGLEWMG | HC A42 |
| | NIYPGSGSTNYAQKFQGRATLTVDTSTSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 113 | QVQLVQSGSEVKKPGASVKVSCKASGYTFTSYWMDWVRQAPGQGLEWMG | VH A43 |
| | NIYPGSGSTNYAQKFQGRATLTADKSTSTAYMELSSLRSEDTAVYYCTRERYGRN | |
| | AMEYWGQGTLVTVSS | |
| 114 | QVQLVQSGSEVKKPGASVKVSCKASGYTFTSYWMDWVRQAPGQGLEWMG | HC A43 |
| | NIYPGSGSTNYAQKFQGRATLTADKSTSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 115 | QVQLVQSGSEVKKPGASVKVSCKASGYTFTSYWMDWVRQAPGQGLEWMG | VH A44 |
| | NIYPGSGSTNYAQKFQGRATLTRDKSTSTAYMELSSLRSEDTAVYYCTRERYGRN | |
| | AMEYWGQGTLVTVSS | |
| 116 | QVQLVQSGSEVKKPGASVKVSCKASGYTFTSYWMDWVRQAPGQGLEWMG | HC A44 |
| | NIYPGSGSTNYAQKFQGRATLTRDKSTSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 117 | QVQLVQSGSEVKKPGASVKVSCKASGYTFTSYWMDWVRQAPGQGLEWMG | VH A45 |
| | NIYPGSGSTNYAQKFQGRATITVDKSTSTAYMELSSLRSEDTAVYYCTRERYGRN | |
| | AMEYWGQGTLVTVSS | |
| 118 | QVQLVQSGSEVKKPGASVKVSCKASGYTFTSYWMDWVRQAPGQGLEWMG | HC A45 |
| | NIYPGSGSTNYAQKFQGRATITVDKSTSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 119 | QVQLVQSGSEVKKPGASVKVSCKASGYTFTSYWMDWVRQAPGQGLEWMG | VH A46 |
| | NIYPGSGSTNYAQKFQGRATMTVDKSTSTAYMELSSLRSEDTAVYYCTRERYGR | |
| | NAMEYWGQGTLVTVSS | |
| 120 | QVQLVQSGSEVKKPGASVKVSCKASGYTFTSYWMDWVRQAPGQGLEWMG | HC A46 |
| | NIYPGSGSTNYAQKFQGRATMTVDKSTSTAYMELSSLRSEDTAVYYCTRERYGR | |
| | NAMEYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 121 | QVQLVQSGSEVKKPGASVKVSCKASGYTFTSYWMDWVRQAPGQGLEWMG | VH A47 |
| | NIYPGSGSTNYAQKFQGRVTLTVDKSTSTAYMELSSLRSEDTAVYYCTRERYGRN | |
| | AMEYWGQGTLVTVSS | |
| 122 | QVQLVQSGSEVKKPGASVKVSCKASGYTFTSYWMDWVRQAPGQGLEWMG | HC A47 |
| | NIYPGSGSTNYAQKFQGRVTLTVDKSTSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 123 | QVQLVQSGSEVKKPGASVKVSCKASGYTFTSYWMDWVRQAPGQGLEWMG | VH A48 |
| | NIYPGSGTTNYAQKFQGRATLTVDKSTSTAYMELSSLRSEDTAVYYCTRERYGRN | |
| | AMEYWGQGTLVTVSS | |
| 124 | QVQLVQSGSEVKKPGASVKVSCKASGYTFTSYWMDWVRQAPGQGLEWMG | HC A48 |
| | NIYPGSGTTNYAQKFQGRATLTVDKSTSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 125 | NIYPSSGSTNYAQKFQG | HCDR2 A49, |
| | | A65-A69 |
| 126 | QVQLVQSGSEVKKPGASVKVSCKASGYTFTSYWMDWVRQAPGQGLEWMG | VH A49 |
| | NIYPSSGSTNYAQKFQGRATLTVDKSTSTAYMELSSLRSEDTAVYYCTRERYGRN | |
| | AMEYWGQGTLVTVSS | |
| 127 | QVQLVQSGSEVKKPGASVKVSCKASGYTFTSYWMDWVRQAPGQGLEWMG | HC A49 |
| | NIYPSSGSTNYAQKFQGRATLTVDKSTSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 128 | NINPGSGSTNYAQKFQG | HCDR2 A50 |
| 129 | QVQLVQSGSEVKKPGASVKVSCKASGYTFTSYWMDWVRQAPGQGLEWMG | VH A50 |
| | NINPGSGSTNYAQKFQGRATLTVDKSTSTAYMELSSLRSEDTAVYYCTRERYGRN | |
| | AMEYWGQGTLVTVSS | |
| 130 | QVQLVQSGSEVKKPGASVKVSCKASGYTFTSYWMDWVRQAPGQGLEWMG | HC A50 |
| | NINPGSGSTNYAQKFQGRATLTVDKSTSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 131 | GYWMD | HCDR1 A51 |
| 132 | QVQLVQSGSEVKKPGASVKVSCKASGYTFTGYWMDWVRQAPGQGLEWMG | VH A51 |
| | NIYPGSGSTNYAQKFQGRATLTVDKSTSTAYMELSSLRSEDTAVYYCTRERYGRN | |
| | AMEYWGQGTLVTVSS | |
| 133 | QVQLVQSGSEVKKPGASVKVSCKASGYTFTGYWMDWVRQAPGQGLEWMG | HC A51 |
| | NIYPGSGSTNYAQKFQGRATLTVDKSTSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 134 | QVQLVQSGTEVKKPGASVKVSCKASGYTFTSYWMDWVRQAPGQGLEWMG | VH A52 |
| | NIYPGSGSTNYAQKFQGRATLTVDKSTSTAYMELSSLRSEDTAVYYCTRERYGRN | |
| | AMEYWGQGTLVTVSS | |
| 135 | QVQLVQSGTEVKKPGASVKVSCKASGYTFTSYWMDWVRQAPGQGLEWMG | HC A52 |
| | NIYPGSGSTNYAQKFQGRATLTVDKSTSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 136 | QVQLVQSGAEVKKPGASVKVSCKASGYTFTSYWMDWVRQAPGQGLEWMG | VH A53 |
| | NIYPGSGSTNYAQKFQGRATLTVDKSTSTAYMELSSLRSEDTAVYYCTRERYGRN | |
| | AMEYWGQGTLVTVSS | |
| 137 | QVQLVQSGAEVKKPGASVKVSCKASGYTFTSYWMDWVRQAPGQGLEWMG | HC A53 |
| | NIYPGSGSTNYAQKFQGRATLTVDKSTSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 138 | DIQMTQSPSSVSASVGDRVTITCRASQTIGTWLAWYQQKPGKSPKLLIYAATSL | VL A56, A59, |
| | ADGVPSRFSGSGSGTDFTLTISSLQPEDFATYYCQQLYSTPLTFGGGTKLEIK | A63, A68, A72, |
| | | A75, A79, A80, |
| | | A83 |
| 139 | DIQMTQSPSSVSASVGDRVTITCRASQTIGTWLAWYQQKPGKSPKLLIYAATSL | LC A56, A59, |
| | ADGVPSRFSGSGSGTDFTLTISSLQPEDFATYYCQQLYSTPLTFGGGTKLEIKRTV | A63, A68, A72, |
| | AAPSVFIFPPSDEQLKSGTASVVCLLNNFYPREAKVQWKVDNALQSGNSQESV | A75, A79, A80, |
| | TEQDSKDSTYSLSSTLTLSKADYEKHKVYACEVTHQGLSSPVTKSFNRGEC | A83 |
| 140 | QVQLVQSGAEVKKPGASVKVSCKASGYTFTSYWMDWVRQAPGQGLEWMG | VH A65-A69 |
| | NIYPSSGSTNYAQKFQGRVTMTVDTSTSTVYMELSSLRSEDTAVYYCARERYGR | |
| | NAAEYWGQGTLVTVSS | |
| 141 | QVQLVQSGAEVKKPGASVKVSCKASGYTFTSYWMDWVRQAPGQGLEWMG | HC A65-A69 |
| | NIYPSSGSTNYAQKFQGRVTMTVDTSTSTVYMELSSLRSEDTAVYYCARERYGR | |
| | NAAEYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 142 | ERYGRNAMDY | HCDR3 A85, |
| | | A87-A91, P |
| 143 | QVQLVQSGSEVKKPGASVKVSCKASGYTFTSYWMDWVRQAPGQGLEWMG | VH A85, A89 |
| | NIYPGSGSTNYAQKFQGRATLTVDKSTSTAYMELSSLRSEDTAVYYCTRERYGRN | |
| | AMDYWGQGTLVTVSS | |
| 144 | QVQLVQSGSEVKKPGASVKVSCKASGYTFTSYWMDWVRQAPGQGLEWMG | HC A85, A89 |
| | NIYPGSGSTNYAQKFQGRATLTVDKSTSTAYMELSSLRSEDTAVYYCTRERYGRN | |
| | AMDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 145 | QVQLVQSGSEVKKPGASVKVSCKASGYTFTSYWMDWVRQAPGKGLEWMG | VH A87, A90 |
| | NIYPGSGSTNYAQKFQGRVTLTVDKATSTAYMELSSLRSEDTATYYCTRERYGRN | |
| | AMDYWGQGTLVTVSS | |
| 146 | QVQLVQSGSEVKKPGASVKVSCKASGYTFTSYWMDWVRQAPGKGLEWMG | HC A87, A90 |
| | NIYPGSGSTNYAQKFQGRVTLTVDKATSTAYMELSSLRSEDTATYYCTRERYGRN | |
| | AMDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 147 | QQYYSTPLT | LCDR3 A98 |
| 148 | DIQMTQSPSSVSASVGDRVTITCLASQTIGTWLAWYQQKPGKSPKLLIYAATSL | VL A98 |
| | ADGVPSRFSGSGSGTDFTFTISSLQPEDFATYYCQQYYSTPLTFGGGTKLEIK | |
| 149 | DIQMTQSPSSVSASVGDRVTITCLASQTIGTWLAWYQQKPGKSPKLLIYAATSL | LC A98 |
| | ADGVPSRFSGSGSGTDFTFTISSLQPEDFATYYCQQYYSTPLTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 150 | ERYGANAMEY | HCDR3 A99 |
| 151 | QVQLVQSGSEVKKPGASVKVSCKASGYTFTSYWMDWVRQAPGQGLEWMG | VH A99 |
| | NIYPGSGSTNYAQKFQGRATLTVDKSTSTAYMELSSLRSEDTAVYYCTRERYGAN | |
| | AMEYWGQGTLVTVSS | |
| 152 | QVQLVQSGSEVKKPGASVKVSCKASGYTFTSYWMDWVRQAPGQGLEWMG | HC A99 |
| | NIYPGSGSTNYAQKFQGRATLTVDKSTSTAYMELSSLRSEDTAVYYCTRERYGAN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 153 | ERAGRNAMEY | HCDR3 A100 |
| 154 | QVQLVQSGSEVKKPGASVKVSCKASGYTFTSYWMDWVRQAPGQGLEWMG | VH A100 |
| | NIYPGSGSTNYAQKFQGRATLTVDKSTSTAYMELSSLRSEDTAVYYCTRERAGRN | |
| | AMEYWGQGTLVTVSS | |
| 155 | QVQLVQSGSEVKKPGASVKVSCKASGYTFTSYWMDWVRQAPGQGLEWMG | HC A100 |
| | NIYPGSGSTNYAQKFQGRATLTVDKSTSTAYMELSSLRSEDTAVYYCTRERAGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 156 | WIYPGSGSTNYAQKFQG | HCDR2 A101 |
| 157 | QVQLVQSGSEVKKPGASVKVSCKASGYTFTSYWMDWVRQAPGQGLEWMG | VH A101 |
| | WIYPGSGSTNYAQKFQGRATLTVDKSTSTAYMELSSLRSEDTAVYYCTRERYGR | |
| | NAMEYWGQGTLVTVSS | |
| 158 | QVQLVQSGSEVKKPGASVKVSCKASGYTFTSYWMDWVRQAPGQGLEWMG | HC A101 |
| | WIYPGSGSTNYAQKFQGRATLTVDKSTSTAYMELSSLRSEDTAVYYCTRERYGR | |
| | NAMEYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 159 | IIYPGSGSTNYAQKFQG | HCDR2 A102 |
| 160 | QVQLVQSGSEVKKPGASVKVSCKASGYTFTSYWMDWVRQAPGQGLEWMGI | VH A102 |
| | IYPGSGSTNYAQKFQGRATLTVDKSTSTAYMELSSLRSEDTAVYYCTRERYGRNA | |
| | MEYWGQGTLVTVSS | |
| 161 | QVQLVQSGSEVKKPGASVKVSCKASGYTFTSYWMDWVRQAPGQGLEWMGI | HC A102 |
| | IYPGSGSTNYAQKFQGRATLTVDKSTSTAYMELSSLRSEDTAVYYCTRERYGRNA | |
| | MEYWGQGTLVTVSSASTKGPSVFPLAPSSKSTSGGTAALGCLVKDYFPEPVTVS | |
| | WNSGALTSGVHTFPAVLQSSGLYSLSSVVTVPSSSLGTQTYICNVNHKPSNTKV | |
| | DKRVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNG | |
| | KEYKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPG | |
| 162 | SYWMH | HCDR1 A103 |
| 163 | QVQLVQSGSEVKKPGASVKVSCKASGYTFTSYWMHWVRQAPGQGLEWMG | VH A103 |
| | NIYPGSGSTNYAQKFQGRATLTVDKSTSTAYMELSSLRSEDTAVYYCTRERYGRN | |
| | AMEYWGQGTLVTVSS | |
| 164 | QVQLVQSGSEVKKPGASVKVSCKASGYTFTSYWMHWVRQAPGQGLEWMG | HC A103 |
| | NIYPGSGSTNYAQKFQGRATLTVDKSTSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 165 | SYYMD | HCDR1 A104 |
| 166 | QVQLVQSGSEVKKPGASVKVSCKASGYTFTSYYMDWVRQAPGQGLEWMGN | VH A104 |
| | IYPGSGSTNYAQKFQGRATLTVDKSTSTAYMELSSLRSEDTAVYYCTRERYGRNA | |
| | MEYWGQGTLVTVSS | |
| 167 | QVQLVQSGSEVKKPGASVKVSCKASGYTFTSYYMDWVRQAPGQGLEWMGN | HC A104 |
| | IYPGSGSTNYAQKFQGRATLTVDKSTSTAYMELSSLRSEDTAVYYCTRERYGRNA | |
| | MEYWGQGTLVTVSSASTKGPSVFPLAPSSKSTSGGTAALGCLVKDYFPEPVTVS | |
| | WNSGALTSGVHTFPAVLQSSGLYSLSSVVTVPSSSLGTQTYICNVNHKPSNTKV | |
| | DKRVEPKSCDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLHQDWLNG | |
| | KEYKCKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPG | |
| 168 | NIYPGSGSTNYDEKFKS | HCDR2 P |
| 169 | QVQLQQPGSELVRPGASVKLSCKASGYTFTSYWMDWVKQRPGQGLEWIGNI | VH P |
| | YPGSGSTNYDEKFKSKATLTVDKSSSTAYMQLSSLTSEDSAVYYCTRERYGRNA | |
| | MDYWGQGTSVTVSS | |
| 170 | DIQMTQSPASQSASLGESVTITCLASQTIGTWLAWYQQKPGKSPQLLIYAATSL | VL P |
| | ADGVPSRFSGSGSGTKFSFKISSLQAEDFVSYYCQQLYSTPLTFGGGTKLEIK | |
| 171 | QVQLVQSGSEVKKPGASVKVSCKASGYTFTSYWMDWVRQAPGQGLEWMG | HC |
| | NIYPGSGSTNYAQKFQGRATLTVDKSTSTAYMELSSLRSEDTAVYYCTRERYGRN | A86+GAALIE |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLAGPSVFLFPPKPKDTLMISRTPEVTCVVVD | |
| | VSHEDPEVKFNWYVDGVEVHNAKTKPREEQYNSTYRVVSVLTVLHQDWLNG | |
| | KEYKCKVSNKALPLPEEKTISKAKGQPREPQVYTLPPSREEMTKNQVSLTCLVKG | |
| | FYPSDIAVEWESNGQPENNYKTTPPVLDSDGSFFLYSKLTVDKSRWQQGNVFS | |
| | CSVMHEALHNHYTQKSLSLSPG | |
| 172 | QVQLVQSGSEVKKPGASVKVSCKASGYTFTSYWMDWVRQAPGQGLEWMG | HC A86+SDIE |
| | NIYPGSGSTNYAQKFQGRATLTVDKSTSTAYMELSSLRSEDTAVYYCTRERYGRN | |
| | AMEYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKRVEPKSCDKTHTCPPCPAPELLGGPDVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPEEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 173 | ASTKGPSVFPLAPSSKSTSGGTAALGCLVKDYFPEPVTVSWNSGALTSGVHTFP | lgG1 CH1-CH2- |
| | AVLQSSGLYSLSSVVTVPSSSLGTQTYICNVNHKPSNTKVDKRVEPKSCDKTHTC | CH3 |
| | PPCPAPELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPAPIE | |
| | KTISKAKGQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALHNHYTQK | |
| | SLSLSPG | |
| 174 | RTVAAPSVFIFPPSDEQLKSGTASVVCLLNNFYPREAKVQWKVDNALQSGNSQ | lgG1 CL |
| | ESVTEQDSKDSTYSLSSTLTLSKADYEKHKVYACEVTHQGLSSPVTKSFNRGEC | |
| 175 | QVQLVQSGSEVKKPGASVKVSCKASGYTFTSYWMDWVRQAPGQGLEWMG | moA85 HC |
| | NIYPGSGSTNYAQKFQGRATLTVDKSTSTAYMELSSLRSEDTAVYYCTRERYGRN | |
| | AMDYWGQGTLVTVSSAKTTAPSVYPLAPVCGDTTGSSVTLGCLVKGYFPEPVT | |
| | LTWNSGSLSSGVHTFPAVLQSDLYTLSSSVTVTSSTWPSQSITCNVAHPASSTKV | |
| | DKKIEPRGPTIKPCPPCKCPAPNLLGGPSVFIFPPKIKDVLMISLSPIVTCVVVDVS | |
| | EDDPDVQISWFVNNVEVHTAQTQTHREDYNSTLRVVSALPIQHQDWMSGKE | |
| | FKCKVNNKDLPAPIERTISKPKGSVRAPQVYVLPPPEEEMTKKQVTLTCMVTDF | |
| | MPEDIYVEWTNNGKTELNYKNTEPVLDSDGSYFMYSKLRVEKKNWVERNSYS | |
| | CSVVHEGLHNHHTTKSFSRTPGK | |
| 176 | DIQMTQSPSSVSASVGDRVTITCLASQTIGTWLAWYQQKPGKSPKLLIYAATSL | moA85 LC |
| | ADGVPSRFSGSGSGTDFTFTISSLQPEDFATYYCQQLYSTPLTFGGGTKLEIKRAD | |
| | AAPTVSIFPPSSEQLTSGGASVVCFLNNFYPKDINVKWKIDGSERQNGVLNSW | |
| | TDQDSKDSTYSMSSTLTLTKDEYERHNSYTCEATHKTSTSPIVKSFNRNEC | |
| 177 | QVQLVQSGSEVKKPGASVKVSCKASGYTFTSYWMDWVRQAPGQGLEWMG | Fab A85 HC |
| | NIYPGSGSTNYAQKFQGRATLTVDKSTSTAYMELSSLRSEDTAVYYCTRERYGRN | |
| | AMDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKKVEPKSCDKENLYFQGSHHHHHH | |
| 178 | AATSLAE | LCDR2 A89- |
| | | A91, A95-A97 |
| 179 | DIQMTQSPSSVSASVGDRVTITCLASQTIGTWLAWYQQKPGKSPKLLIYAATSL | VL A89-A91, |
| | AEGVPSRFSGSGSGTDFTFTISSLQPEDFATYYCQQLYSTPLTFGGGTKLEIK | A95-A97 |
| 180 | DIQMTQSPSSVSASVGDRVTITCLASQTIGTWLAWYQQKPGKSPKLLIYAATSL | LC A89-A91, |
| | AEGVPSRFSGSGSGTDFTFTISSLQPEDFATYYCQQLYSTPLTFGGGTKLEIKRTV | A95-A97 |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| 181 | QVQLVQSGSEVKKPGASVKVSCKASGYTFTSYWMDWVRQAPGKGLEWMG | VH A88, A91 |
| | NIYPGSGSTNYAQKFQGRVTLTVDKSTDTAYMELSSLRSEDTATYYCTRERYGRN | |
| | AMDYWGQGTLVTVSS | |
| 182 | QVQLVQSGSEVKKPGASVKVSCKASGYTFTSYWMDWVRQAPGKGLEWMG | HC A88, A91 |
| | NIYPGSGSTNYAQKFQGRVTLTVDKSTDTAYMELSSLRSEDTATYYCTRERYGRN | |
| | AMDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 183 | ERYGRQAMDY | HCDR3 A92- |
| | | A97 |
| 184 | QVQLVQSGSEVKKPGASVKVSCKASGYTFTSYWMDWVRQAPGQGLEWMG | VH A92, A95 |
| | NIYPGSGSTNYAQKFQGRATLTVDKSTSTAYMELSSLRSEDTAVYYCTRERYGRQ | |
| | AMDYWGQGTLVTVSS | |
| 185 | QVQLVQSGSEVKKPGASVKVSCKASGYTFTSYWMDWVRQAPGQGLEWMG | HC A92, A95 |
| | NIYPGSGSTNYAQKFQGRATLTVDKSTSTAYMELSSLRSEDTAVYYCTRERYGRQ | |
| | AMDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 186 | QVQLVQSGSEVKKPGASVKVSCKASGYTFTSYWMDWVRQAPGKGLEWMG | VH A93, A96 |
| | NIYPGSGSTNYAQKFQGRVTLTVDKATSTAYMELSSLRSEDTATYYCTRERYGRQ | |
| | AMDYWGQGTLVTVSS | |
| 187 | QVQLVQSGSEVKKPGASVKVSCKASGYTFTSYWMDWVRQAPGKGLEWMG | HC A93, A96 |
| | NIYPGSGSTNYAQKFQGRVTLTVDKATSTAYMELSSLRSEDTATYYCTRERYGRQ | |
| | AMDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 188 | QVQLVQSGSEVKKPGASVKVSCKASGYTFTSYWMDWVRQAPGKGLEWMG | VH A94, A97 |
| | NIYPGSGSTNYAQKFQGRVTLTVDKSTDTAYMELSSLRSEDTATYYCTRERYGRQ | |
| | AMDYWGQGTLVTVSS | |
| 189 | QVQLVQSGSEVKKPGASVKVSCKASGYTFTSYWMDWVRQAPGKGLEWMG | HC A94, A97 |
| | NIYPGSGSTNYAQKFQGRVTLTVDKSTDTAYMELSSLRSEDTATYYCTRERYGRQ | |
| | AMDYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTK | |
| | VDKRVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPG | |
| 190 | RLLSGQPVCRGGTQRPCYKVIYFHDTSRRLNFEEAKEACRRD | LAYN-v2-His |
| | GGQLVSIESEDEQKLIEKFIENLLPSDGDFWIGLRRREEKQSNS | |
| | TACQDLYAWTDGSISQFRNWYVDEPSCGSEVCVVMYHQPSA | |
| | PAGIGGPYMFQWNDDRCNMKNNFICKYSDEKPAVPSREAEG | |
| | EETELTTPVLPEETQEEDAKKTFKESREAALNLAYGGHHHHHH | |
| | HHHH | |
| 191 | RLLSGQPVCRGGTQRPCYKVIYFHDTSRRLNFEEAKEACRRD | LAYN-v2-no-tag |
| | GGQLVSIESEDEQKLIEKFIENLLPSDGDFWIGLRRREEKQSNS | |
| | TACQDLYAWTDGSISQFRNWYVDEPSCGSEVCVVMYHQPSA | |
| | PAGIGGPYMFQWNDDRCNMKNNFICKYSDEKPAVPSREAEG | |
| | EETELTTPVLPEETQEEDAKKTFKESREAALNLAY | |
| 192 | RLLSGQPVCRGGTQRPCYKVIYFHDTSRRLNFEEAKEACRRD | LAYN v2 22- |
| | GGQLVSIESEDEQKLIEKFIENLLPSDGDFWIGLRRREEKQSNS | 179-His |
| | TACQDLYAWTDGSISQFRNWYVDEPSCGSEVCVVMYHQPSA | |
| | PAGIGGPYMFQWNDDRCNMKNNFICKYSDGGHHHHHHHHH | |
| | H | |
| 193 | MRPGTALQAVLLAVLLVGLRAATGRLLSGQPVCRGGTQRPCY | FL-LAYN-SP v2 |
| | KVIYFHDTSRRLNFEEAKEACRRDGGQLVSIESEDEQKLIEKFI | |
| | ENLLPSDGDFWIGLRRREEKQSNSTACQDLYAWTDGSISQFR | |
| | NWYVDEPSCGSEVCVVMYHQPSAPAGIGGPYMFQWNDDRC | |
| | NMKNNFICKYSDEKPAVPSREAEGEETELTTPVLPEETQEEDA | |
| | KKTFKESREAALNLAYILIPSIPLLLLLVVTTVVCWVWICRKRKR | |
| | EQPDPSTKKQHTIWPSPHQGNSPDLEVYNVIRKQSEADLAET | |
| | RPDLKNISFRVCSGEATPDDMSCDYDNMAVNPSESGFVTLVS | |
| | VESGFVTNDIYEFSPDQMGRSKESGWVENEIYGY | |
| 194 | RLLSGQPVCRGGTQRPCYKVIYFHDTSRRLNFEEAKEACRRD | FL-LAYN v2 |
| | GGQLVSIESEDEQKLIEKFIENLLPSDGDFWIGLRRREEKQSNS | |
| | TACQDLYAWTDGSISQFRNWYVDEPSCGSEVCVVMYHQPSA | |
| | PAGIGGPYMFQWNDDRCNMKNNFICKYSDEKPAVPSREAEG | |
| | EETELTTPVLPEETQEEDAKKTFKESREAALNLAYILIPSIPLLLL | |
| | LVVTTVVCWVWICRKRKREQPDPSTKKQHTIWPSPHQGNSPD | |
| | LEVYNVIRKQSEADLAETRPDLKNISFRVCSGEATPDDMSCDY | |
| | DNMAVNPSESGFVTLVSVESGFVTNDIYEFSPDQMGRSKESG | |
| | WVENEIYGY | |
| 195 | MRPGTALQAVLLAVLLVGLRAATGRLLSGQPVCRGGTQRPCY | LAYN v2 ECD1- |
| | KVIYFHDTSRRLNFEEAKEACRRDGGQLVSIESEDEQKLIEKFI | 250 |
| | ENLLPSDGDFWIGLRRREEKQSNSTACQDLYAWTDGSISQFR | |
| | NWYVDEPSCGSEVCVVMYHQPSAPAGIGGPYMFQWNDDRC | |
| | NMKNNFICKYSDEKPAVPSREAEGEETELTTPVLPEETQEEDA | |
| | KKTFKESREAALNLAYILIPSIPLLLLLVVTTVVCWVWI | |
| 196 | RLLSASDLDLRGGQPVCRGGTQRPCYKVIYFHDTSRRLNFEE | LAYN-v1-His |
| | AKEACRRDGGQLVSIESEDEQKLIEKFIENLLPSDGDFWIGLRR | |
| | REEKQSNSTACQDLYAWTDGSISQFRNWYVDEPSCGSEVCV | |
| | VMYHQPSAPAGIGGPYMFQWNDDRCNMKNNFICKYSDEKPA | |
| | VPSREAEGEETELTTPVLPEETQEEDAKKTFKESREAALNLAY | |
| | GGHHHHHHHHHH | |
| 197 | RLLSASDLDLRGGQPVCRGGTQRPCYKVIYFHDTSRRLNFEE | LAYN-v1 ECD |
| | AKEACRRDGGQLVSIESEDEQKLIEKFIENLLPSDGDFWIGLRR | |
| | REEKQSNSTACQDLYAWTDGSISQFRNWYVDEPSCGSEVCV | |
| | VMYHQPSAPAGIGGPYMFQWNDDRCNMKNNFICKYSDEKPA | |
| | VPSREAEGEETELTTPVLPEETQEEDAKKTFKESREAALNLAY | |
| 198 | MVTSGLGSGGVRRNKAIAQPARTFMLGLMAAYHNLEKPAVPS | LAYN-v3 ECD |
| | REAEGEETELTTPVLPEETQEEDAKKTFKESREAALNLAY | |
| 199 | MYHQPSAPAGIGGPYMFQWNDDRCNMKNNFICKYSDEKPAV | LAYN-v4 ECD |
| | PSREAEGEETELTTPVLPEETQEEDAKKTFKESREAALNLAY | |
| 200 | RLLSGQPVCRGGTQRPCYKVIYFHDTSRRLNFEEAKEACRRD | LAYN-v2 ECD |
| | GGQLVSIESEDEQNLIEKFIENLLPSDGDFWIGLRRREEKQSNS | (Macaca |
| | TACEDLYAWTDGSISQFRNWYVDEPSCGSEVCVVMYHQPSA | fascicularis) |
| | PAGIGGPYMFQWNDDRCNMKNNFICKYSDEKPAVPSGEPEG | |
| | EETEPTTPVLLEETQEEDAKKTFKESREAALNLAY | |
| 201 | RLLSGQPVCREGTQRPCYKVIYFHDASRRLNFEEANAACRTD | LAYN ECD |
| | GGQLLSIESQDEQRLIEKFIENLLASDGDFWIGLRRREERQSN | (Canis lupus |
| | STACQDLYAWTDGSTSAFRNWYVDEPSCGSEVCVVMYHQPS | familiaris) |
| | APAGVGGLYMFQWNDDRCNMKNNFICKYSDEKPTAPSIKPGG | |
| | EGTEPATPILPGDAGKEDARETFKGNNGMDRTLQGKIEAALNL | |
| | AY | |
| 202 | HLLSGQRFCWEGTRRPCYRVIYFHDTSQRRNFEEAKEACMK | LAYN ECD |
| | DGGQLASIETADEQRLIENFIESLQASDGDFWIGLKRLEEKQHN | (Rattus |
| | STACQDLYAWTDGSLSQFRNWYMDEPSCESEVCVVMYHQPS | norvegicus) |
| | APPGIGGPYMFQWNDDRCNTKKNFICKYSDDKPSTTPSLSPG | |
| | GEATEPAAPILPEETLEIDTKERREAALNLAY | |
| 203 | RLLSGQLVCRGGTRRPCYKVIYFHDAFQRLNFEEAKETCMED | LAYN ECD |
| | GGQLVSIETEDEQRLIEKFIENLLASDGDFWIGLKRLEEKQSNN | (Mus musculus) |
| | TACQDLYAWTDGSTSQFRNWYVDEPSCGSEVCVVMYHQPSA | |
| | PPGIGGSYMFQWNDDRCNMKNNFICKYHDDKPSTTPSIWPG | |
| | GEATEPATPLLPEETQKEDTKETFKERREAALNLAY | |
| 204 | RLLSGQLVCRGGTQRPCYKVIYFHDASRRLNFEEAKEACRRD | LAYN ECD |
| | SGQLVSIESEDEQRLIEKFIENLLASDGDFWIGLRRQEEKQNNR | A182V |
| | TACQDLYSWTDGSSSKFRNWYVDEPSCGSEVCVVMYHQPSA | (Oryctolagus |
| | PAGIGGPYMFQWNDDRCNMKNNFICKYSDEKPTPPSTRLGG | cuniculus) |
| | AATEPATPILPEEVQKEDAKETFKESKEAALNLAY | |
| 205 | RLLSGQPVCREGTRRPCYEVIYFQESSRRLNFEEARAACGRD | LAYN ECD |
| | GGQLVSIESEDEQRLIEKFIENLHASDGDFWIGLRRWEEKQSN | (Sus scrofa) |
| | STACQDLYAWTDGSTATFRNWYADEPSCGSEVCVVMYHQPS | |
| | APAGIGGRYMFQWNDDRCTMKNNFICKYSEEKPTAPPTRPGG | |
| | EGTEPATPRLPEDTEKEEATGTLKESSEAALNLAY | |
| 206 | X₁YWMD | Consensus |
| | wherein | HCDR1 |
| | X₁ may be any amino acid, preferably S or G, more preferably S | |
| 207 | NIX₂PX₃SGX₄TNYX₅X₆KFX₇X₈ | Consensus |
| | wherein | HCDR2 |
| | X₂ may be any amino acid, preferably Y or N, more preferably Y; | |
| | X₃ may be any amino acid, preferably S or G, more preferably S; | |
| | X₄ may be any amino acid, preferably T or S, more preferably T, | |
| | X₅ may be any amino acid, preferably A or D, more preferably A; | |
| | X₆ may be any amino acid, preferably Q or E, more preferably Q; | |
| | X₇ may be any amino acid, preferably Q or K, more preferably Q; | |
| | and | |
| | X₈ may be any amino acid, preferably G or S, more preferably G | |
| 208 | X₉X₁₀YX₁₁RX₁₂AX₁₃X₁₄X₁₅ | Consensus |
| | wherein | HCDR3 |
| | X₉ may be any amino acid, preferably E or A, more preferably E; | |
| | X₁₀ may be any amino acid, preferably R, K or A, more preferably | |
| | R; | |
| | X₁₁ may be any amino acid, preferably G or A, more preferably G; | |
| | X₁₂ may be any amino acid, preferably N or A, more preferably N; | |
| | X₁₃ may be any amino acid, preferably A or M, more preferably A; | |
| | X₁₄ may be any amino acid, preferably E, D or A, more preferably | |
| | E; and | |
| | X₁₅ may be any amino acid, preferably Y or A, more preferably Y | |
| 209 | X₁₆ASQX₁₇1X₁₈X₁₉X₂₀LA | Consensus |
| | wherein | LCDR1 |
| | X₁₆ may be any amino acid, preferably R or L, more preferably R; | |
| | X₁₇ may be any amino acid, preferably G, T or S, more preferably | |
| | G; | |
| | X₁₈ may be any amino acid, preferably G or S, more preferably G; | |
| | X₁₉ may be any amino acid, preferably S or T, more preferably S; | |
| | and | |
| | X₂₀ may be any amino acid, preferably W or Y, more preferably | |
| | W | |
| 210 | AAX₂₁SLX₂₂X₂₃ | Consensus |
| | wherein | LCDR2 |
| | X₂₁ may be any amino acid, preferably T or S, more preferably T; | |
| | X₂₂ may be any amino acid, preferably A or Q, more preferably A; | |
| | and | |
| | X₂₃ may be any amino acid, preferably D, T, S or E, more | |
| | preferably D | |
| 211 | QQX₂₄YSX₂₅PLT | Consensus |
| | wherein | LCDR3 |
| | X₂₄ is L, A or S, preferably L; and | |
| | X₂₅ may be any amino acid, preferably T or F, more preferably T | |
| 212 | QVQLX₂₆QX₂₇GX₂₈EX₂₉X₃₀X₃₁PGASVKX₃₂SCKASGYTFTX1YW | Consensus VH |
| | MDWVX₃₃QX₃₄PGX₃₅GLEWX₃₆GNIX2PX3SGX4TNYX5X6KFX7X8 | |
| | X₃₇X₃₈TX₃₉TX₄₀DX⁴¹X⁴²X⁴³X⁴⁴TX⁴⁵YMX⁴⁶LSX⁴⁷LX⁴⁸SX⁴⁹DX⁵⁰AX⁵¹ | |
| | YYCX⁵²RX⁹X¹⁰YX¹¹RX¹²AX¹³X¹⁴X¹⁵WGQGTX53 VTVSS | |
| | wherein | |
| | X₁ may be any amino acid, preferably S or G, more preferably S, | |
| | X₂ may be any amino acid, preferably Y or N, more preferably Y, | |
| | X₃ may be any amino acid, preferably G or S, more preferably S, | |
| | X₄ may be any amino acid, preferably S or T, more preferably T, | |
| | X₅ may be any amino acid, preferably D or A, more preferably A, | |
| | X₆ may be any amino acid, preferably E or Q, more preferably Q, | |
| | X₇ may be any amino acid, preferably K or Q, more preferably Q, | |
| | X₈ may be any amino acid, preferably S or G, more preferably G, | |
| | X₉ may be any amino acid, preferably E or A, more preferably E, | |
| | X₁₀ may be any amino acid, preferably R, K or A, more preferably | |
| | R, | |
| | X₁₁ may be any amino acid, preferably G or A, more preferably G, | |
| | X₁₂ may be any amino acid, preferably N or A, more preferably N, | |
| | X₁₃ may be any amino acid, preferably M or A, more preferably A, | |
| | X₁₄ may be any amino acid, preferably D, E or A, more preferably | |
| | E, | |
| | X₁₅ may be any amino acid, preferably Y or A, more preferably Y, | |
| | X₂₆ may be any amino acid, preferably V or Q, more preferably V, | |
| | X₂₇ may be any amino acid, preferably S or P, more preferably S, | |
| | X₂₈ may be any amino acid, preferably A, S or T, more preferably | |
| | A, | |
| | X₂₉ may be any amino acid, preferably V or L, more preferably V, | |
| | X₃₀ may be any amino acid, preferably K or V, more preferably K, | |
| | X₃₁ may be any amino acid, preferably Kor R, more preferably K, | |
| | X₃₂ may be any amino acid, preferably V or L, more preferably V, | |
| | X₃₃ may be any amino acid, preferably R or K, more preferably R, | |
| | X₃₄ may be any amino acid, preferably A or R, more preferably A, | |
| | X₃₅ may be any amino acid, preferably Q or K, more preferably Q, | |
| | X₃₆ may be any amino acid, preferably M or I, more preferably M, | |
| | X₃₇ may be any amino acid, preferably R or K, more preferably R, | |
| | X₃₈ may be any amino acid, preferably V or A, more preferably V, | |
| | X₃₉ may be any amino acid, preferably M, L or I, more preferably | |
| | M, | |
| | X₄₀ may be any amino acid, preferably V, A or R, more preferably | |
| | V, | |
| | X₄₁ may be any amino acid, preferably K or T, more preferably K, | |
| | X₄₂ may be any amino acid, preferably S or A, more preferably S, | |
| | X₄₃ may be any amino acid, preferably T, K, I or S, more | |
| | preferably T, | |
| | X₄₄ may be any amino acid, preferably S or D, more preferably S, | |
| | X₄₅ may be any amino acid, preferably A, L or V, more preferably | |
| | A, | |
| | X₄₆ may be any amino acid, preferably E or Q, more preferably E, | |
| | X₄₇ may be any amino acid, preferably S or R, more preferably S, | |
| | X₄₈ may be any amino acid, preferably R or T, more preferably R, | |
| | X₄₉ may be any amino acid, preferably E or D, more preferably E, | |
| | X₅₀ may be any amino acid, preferably T or S, more preferably T, | |
| | X₅₁ may be any amino acid, preferably V or T, more preferably V, | |
| | X₅₂ may be any amino acid, preferably A or T, more preferably A, | |
| | and | |
| | X₅₃ may be any amino acid, preferably L or S, more preferably L | |
| 213 | DIQMTQSPX₅₄SX₅₅SASX₅₆GX₅₇X₅₈VTITCX₁₆ASQX₁₇1X₁₈X₁₉X₂₀L | Consensus VL |
| | AWYQQKPGKX₅₉PX₆₀LLIYAAX₂₁SLX₂₂X₂₃GVPSRFSGSGSGTX | |
| | ₆₁FX₆₂X₆₃X₆₄ISSLQX₆₅EDFX₆₆X₆₇YYCQQX₂₄YSX₂₅PLTFGGGTK | |
| | LEIK | |
| | wherein | |
| | X₁₆ may be any amino acid, preferably L or R, more preferably R, | |
| | X₁₇ may be any amino acid, preferably T, G or S, more preferably | |
| | G, | |
| | X₁₈ may be any amino acid, preferably G or S, more preferably G, | |
| | X₁₉ may be any amino acid, preferably T or S, more preferably S, | |
| | X₂₀ may be any amino acid, preferably W or Y, more preferably W, | |
| | X₂₁ may be any amino acid, preferably T or S, more preferably T, | |
| | X₂₂ may be any amino acid, preferably A or Q, more preferably A, | |
| | X₂₃ may be any amino acid, preferably D, T, S or E, more | |
| | preferably D, | |
| | X₂₄ is L, A or S, preferably L, | |
| | X₂₅ may be any amino acid, preferably T or F, more preferably T, | |
| | X₅₄ may be any amino acid, preferably S or A, more preferably S, | |
| | X₅₅ may be any amino acid, preferably V, L or Q, more preferably | |
| | V, | |
| | X₅₆ may be any amino acid, preferably V or L, more preferably V, | |
| | X₅₇ may be any amino acid, preferably D or E, more preferably D, | |
| | X₅₈ may be any amino acid, preferably R or S, more preferably R, | |
| | X₅₉ may be any amino acid, preferably A or S, more preferably A, | |
| | X₆₀ may be any amino acid, preferably K or Q, more preferably K, | |
| | X₆₁ may be any amino acid, preferably D or K, more preferably D, | |
| | X₆₂ may be any amino acid, preferably T or S, more preferably T, | |
| | X₆₃ may be any amino acid, preferably L or F, more preferably L, | |
| | X₆₄ may be any amino acid, preferably T or K, more preferably T, | |
| | X₆₅ may be any amino acid, preferably P or A, more preferably P, | |
| | X₆₆ may be any amino acid, preferably A or V, more preferably A, | |
| | and | |
| | X₆₇ may be any amino acid, preferably T or S, more preferably T | |
| 214 | QTIGTY | LCDR1 |
| | | AHo/IMGT A21 |
| 215 | QTIGSW | LCDR1 |
| | | AHo/IMGT A22 |
| 216 | QTISTW | LCDR1 |
| | | AHo/IMGT A23 |
| 217 | QGIGTW | LCDR1 |
| | | AHo/IMGT A24 |
| 218 | QSIGTW | LCDR1 |
| | | AHo/IMGT A25 |
| 219 | MYPPY | CTLA-4 |
| | | sequence motif |
| 220 | MRPGTALQAVLLAVLLVGLRAATGRLLSASDLDLRGGQPVCRG | FL-LAYN-SP v1 |
| | GTQRPCYKVIYFHDTSRRLNFEEAKEACRRDGGQLVSIESEDE | |
| | QKLIEKFIENLLPSDGDFWIGLRRREEKQSNSTACQDLYAWTD | |
| | GSISQFRNWYVDEPSCGSEVCVVMYHQPSAPAGIGGPYMFQ | |
| | WNDDRCNMKNNFICKYSDEKPAVPSREAEGEETELTTPVLPE | |
| | ETQEEDAKKTFKESREAALNLAYILIPSIPLLLLLVVTTVVCWVW | |
| | ICRKRKREQPDPSTKKQHTIWPSPHQGNSPDLEVYNVIRKQS | |
| | EADLAETRPDLKNISFRVCSGEATPDDMSCDYDNMAVNPSES | |
| | GFVTLVSVESGFVTNDIYEFSPDQMGRSKESGWVENEIYGY | |
| 221 | MVTSGLGSGGVRRNKAIAQPARTFMLGLMAAYHNLEKPAVPS | FL-LAYN-SP v3 |
| | REAEGEETELTTPVLPEETQEEDAKKTFKESREAALNLAYILIPS | |
| | IPLLLLLVVTTVVCWVWICRKRKREQPDPSTKKQHTIWPSPHQ | |
| | GNSPDLEVYNVIRKQSEADLAETRPDLKNISFRVCSGEATPDD | |
| | MSCDYDNMAVNPSESGFVTLVSVESGFVTNDIYEFSPDQMGR | |
| | SKESGWVENEIYGY | |
| 222 | MYHQPSAPAGIGGPYMFQWNDDRCNMKNNFICKYSDEKPAV | FL-LAYN-SP v4 |
| | PSREAEGEETELTTPVLPEETQEEDAKKTFKESREAALNLAYILI | |
| | PSIPLLLLLVVTTVVCWVWICRKRKREQPDPSTKKQHTIWPSP | |
| | HQGNSPDLEVYNVIRKQSEADLAETRPDLKNISFRVCSGEATP | |
| | DDMSCDYDNMAVNPSESGFVTLVSVESGFVTNDIYEFSPDQM | |
| | GRSKESGWVENEIYGY | |
| 223 | DYKDDDDK | Antibody tag |
| 224 | DIQMTQSPSSVSASVGDRVTITCRASQGISSWLAWYQQKPGK | VL human |
| | APKLLIYAASSLQSGVPSRFSGSGSGTDFTLTISSLQPEDFATY | germline |
| | YCQQANSFPLTFGGGTKLEIK | sequence |
| 225 | QVQLVQSGAEVKKPGASVKVSCKASGYTFTSYYMHWVRQAP | VH human |
| | GQGLEWMGIINPSGGSTSYAQKFQGRVTMTRDTSTSTVYMEL | germline |
| | SSLRSEDTAVYYCARERYGRNAMEYWGQGTLVTVSS | sequence |
| 226 | GYTFTSYWMD | HCDR1 CCG |
| | | A1-A50, A52- |
| | | A102, P |
| 227 | GYTFTGYWMD | HCDR1 CCG |
| | | A51 |
| 228 | GYTFTSYWMH | HCDR1 CCG |
| | | A103 |
| 229 | GYTFTSYYMD | HCDR1 CCG |
| | | A104 |
| 230 | GYTFTSYW | HCDR1 |
| | | AHo/IMGT A1- |
| | | A50, A52-A103, |
| | | P |
| 231 | GYTFTGYW | HCDR1 |
| | | AHo/IMGT A51 |
| 232 | GYTFTSYY | HCDR1 |
| | | AHo/IMGT |
| | | A104 |
| 233 | GYTFTSY | HCDR1 |
| | | Chothia A1- |
| | | A50, A52-A104, |
| | | P |
| 234 | GYTFTGY | HCDR1 |
| | | Chothia A51 |
| 235 | IYPSSGTT | HCDR2 |
| | | AHo/IMGT A1, |
| | | A3, A57-A64 |
| 236 | IYPGSGTT | HCDR2 |
| | | AHo/IMGT A2, |
| | | A7, A48, A77- |
| | | A79 |
| 237 | IYPGSGST | HCDR2 |
| | | AHo/IMGT A4- |
| | | A6, A8-A47, |
| | | A51-A56, A70- |
| | | A76, A80-A100- |
| | | A104, P |
| 238 | IYPSSGST | HCDR2 |
| | | AHo/IMGT A49, |
| | | A65-A69 |
| 239 | INPGSGST | HCDR2 |
| | | AHo/IMGT A50 |
| 240 | YPSSGT | HCDR2 |
| | | Chothia A1, A3, |
| | | A57-A64 |
| 241 | YPGSGT | HCDR2 |
| | | Chothia A2, A7, |
| | | A48, A77-A79 |
| 242 | YPGSGS | HCDR2 |
| | | Chothia A4-A6, |
| | | A8-A47, A51- |
| | | A56, A70-A76, |
| | | A80-A100- |
| | | A104, P |
| 243 | YPSSGS | HCDR2 |
| | | Chothia A49, |
| | | A65-A69 |
| 244 | NPGSGS | HCDR2 |
| | | Chothia A50 |
| 245 | ARERYGRNAAEY | HCDR3 |
| | | AHo/IMGT A1- |
| | | A7, A57-A79 |
| 246 | TRERYGRNAAEY | HCDR3 |
| | | AHo/IMGT A8, |
| | | A9, A30, A80, |
| | | A81 |
| 247 | TREKYGRNAMEY | HCDR3 |
| | | AHo/IMGT A10, |
| | | A11, A82-A84 |
| 248 | TRERYGRNAMEY | HCDR3 |
| | | AHo/IMGT A12- |
| | | A27, A36-A56, |
| | | A86, A98, |
| | | A101-A104 |
| 249 | TRERYGRNAMEA | HCDR3 |
| | | AHo/IMGT A28 |
| 250 | TRERYGRNAMAY | HCDR3 |
| | | AHo/IMGT A29 |
| 251 | TRERYGRAAMEY | HCDR3 |
| | | AHo/IMGT A31 |
| 252 | TRERYARNAMEY | HCDR3 |
| | | AHo/IMGT A32 |
| 253 | TREAYGRNAMEY | HCDR3 |
| | | AHo/IMGT A33 |
| 254 | TRARYGRNAMEY | HCDR3 |
| | | AHo/IMGT A34 |
| 255 | ARERYGRNAMEY | HCDR3 |
| | | AHo/IMGT A35 |
| 256 | TRERYGRNAMDY | HCDR3 |
| | | AHo/IMGT A85, |
| | | A87-A91, P |
| 257 | TRERYGRQAMDY | HCDR3 |
| | | AHo/IMGT A92- |
| | | A97 |
| 258 | TRERYGANAMEY | HCDR3 |
| | | AHo/IMGT A99 |
| 259 | TRERAGRNAMEY | HCDR3 |
| | | AHo/IMGT |
| | | A100 |
| 260 | RYGRNAAE | HCDR3 |
| | | Chothia A1-A9, |
| | | A30, A57-A81 |
| 261 | KYGRNAME | HCDR3 |
| | | Chothia A10, |
| | | A11, A82-A84 |
| 262 | RYGRNAME | HCDR3 |
| | | Chothia A12- |
| | | A27, A28, A34- |
| | | A56, A86, A98, |
| | | A101-A104 |
| 263 | RYGRNAMA | HCDR3 |
| | | Chothia A29 |
| 264 | RYGRAAME | HCDR3 |
| | | Chothia A31 |
| 265 | RYARNAME | HCDR3 |
| | | Chothia A32 |
| 266 | AYGRNAME | HCDR3 |
| | | Chothia A33 |
| 267 | RYGRNAMD | HCDR3 |
| | | Chothia A85, |
| | | A87-A91, P |
| 268 | RYGRQAMD | HCDR3 |
| | | Chothia A92- |
| | | A97 |
| 269 | RYGANAME | HCDR3 |
| | | Chothia A99 |
| 270 | RAGRNAME | HCDR3 |
| | | Chothia A100 |
| 271 | QGIGSW | LCDR1 |
| | | AHo/IMGT A1, |
| | | A3, A5, A8, |
| | | A10, A54, A66, |
| | | A71, A78 |
| 272 | QTIGTW | LCDR1 |
| | | AHo/IMGT A2, |
| | | A4, A6, A7, A9, |
| | | A11-A20, A26- |
| | | A53, A55-A65, |
| | | A67-A70, A72- |
| | | A77, A79-A104, |
| | | P |

1. An anti-LAYN antibody, or an antigen-binding fragment thereof, which binds to an epitope of the extracellular domain of human LAYN isoform 2, wherein the epitope comprises (i) amino acid residues comprised in the loop of human LAYN isoform 2 containing residues 123-138 of the amino acid sequence as set forth in SEQ ID NO: 191, and (ii) amino acid residues comprised in the helix of human LAYN isoform 2 containing residues 52-67 of the amino acid sequence as set forth in SEQ ID NO: 191; preferably wherein the epitope comprises (i) amino acid residues H123, P125 and A127 of the amino acid sequence as set forth in SEQ ID NO: 191, and (ii) amino acid residue E63 of the amino acid sequence as set forth in SEQ ID NO: 191.

2. An anti-LAYN antibody, or an antigen-binding fragment thereof, comprising:
a heavy chain variable region comprising:
a heavy chain complementary determining region 1 (HCDR1) having the amino acid sequence of X₁YWMD (SEQ ID NO: 206), wherein
X₁ is preferably S or G, more preferably S;
a heavy chain complementary determining region 2 (HCDR2) having the amino acid sequence of NIX₂PX₃SGX₄TNYX₅X₆KFX₇X₈ (SEQ ID NO: 207), wherein
X₂ is preferably Y or N, more preferably Y;
X₃ is preferably S or G, more preferably S;
X₄ is preferably T or S, more preferably T,
X₅ is preferably A or D, more preferably A;
X₆ is preferably Q or E, more preferably Q;
X₇ is preferably Q or K, more preferably Q; and
X₈ is preferably G or S, more preferably G; and
a heavy chain complementary determining region 3 (HCDR3) having the amino acid sequence of X₉X₁₀YX₁₁RX₁₂AX₁₃X₁₄X₁₅ (SEQ ID NO: 208), wherein
X₉ is preferably E or A, more preferably E;
X₁₀ is preferably R, K or A, more preferably R;
X₁₁ is preferably G or A, more preferably G;
X₁₂ is preferably N or A, more preferably N;
X₁₃ is preferably A or M, more preferably A;
X₁₄ is preferably E, D or A, more preferably E; and
X₁₅ is preferably Y or A, more preferably Y; and
a light chain variable region comprising
a light chain complementary determining region 1 (LCDR1) having the amino acid sequence of X₁₆ASQX₁₇₁X₁₈X₁₉X₂₀LA (SEQ ID NO: 209), wherein
X₁₆ is preferably R or L, more preferably R;
X₁₇ is preferably G, T or S, more preferably G;
X₁₈ is preferably G or S, more preferably G;
X₁₉ is preferably S or T, more preferably S; and
X₂₀ is preferably W or Y, more preferably W;
a light chain complementary determining region 2 (LCDR2) having the amino acid sequence of AAX₂₁SLX₂₂X₂₃ (SEQ ID NO: 210), wherein
X₂₁ is preferably T or S, more preferably T;
X₂₂ is preferably A or Q, more preferably A; and
X₂₃ is preferably D, T, S or E, more preferably D; and
a light chain complementary determining region 3 (LCDR3) having the amino acid sequence of QQX₂₄YSX₂₅PLT (SEQ ID NO: 211), wherein
X₂₄ is L, A or S, preferably L; and
X₂₅ is preferably T or F, more preferably T.

3. The anti-LAYN antibody, or an antigen-binding fragment thereof, according to claim 1 or 2, wherein the antibody or the antigen-binding fragment comprises:
a heavy chain variable region comprising an HCDR1 having the amino acid sequence of SEQ ID NO: 1, an HCDR2 having the amino acid sequence of any one of SEQ ID NOs 2, 11, or 19, and an HCDR3 having the amino acid sequence of any one of SEQ ID NOs 3, 32, or 142; and
a light chain variable region comprising an LCDR1 having the amino acid sequence of any one of SEQ ID NOs 4, 12, or 24, an LCDR2 having the amino acid sequence of SEQ ID NO: 5, and an LCDR3 having the amino acid sequence of SEQ ID NO: 6.

4. The anti-LAYN antibody, or an antigen-binding fragment thereof, according to claim 3, wherein the antibody or the antigen-binding fragment comprises:
(i) a heavy chain variable region comprising an HCDR1 having the amino acid sequence of SEQ ID NO: 1, an HCDR2 having the amino acid sequence of SEQ ID NO: 2, and an HCDR3 having the amino acid sequence of SEQ ID NO: 3; and a light chain variable region comprising an LCDR1 having the amino acid sequence of SEQ ID NO: 4, an LCDR2 having the amino acid sequence of SEQ ID NO: 5, and an LCDR3 having the amino acid sequence of SEQ ID NO: 6; or
(ii) a heavy chain variable region comprising an HCDR1 having the amino acid sequence of SEQ ID NO: 1, an HCDR2 having the amino acid sequence of SEQ ID NO: 11, and an HCDR3 having the amino acid sequence of SEQ ID NO: 3; and a light chain variable region comprising an LCDR1 having the amino acid sequence of SEQ ID NO: 12, an LCDR2 having the amino acid sequence of SEQ ID NO: 5, and an LCDR3 having the amino acid sequence of SEQ ID NO: 6; or
(iii) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 142; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6; or
(iv) a heavy chain variable region comprising an HCDR1 having the amino acid sequence of SEQ ID NO: 1, an HCDR2 having the amino acid sequence of SEQ ID NO: 19, and an HCDR3 having the amino acid sequence of SEQ ID NO: 32; and a light chain variable region comprising an LCDR1 having the amino acid sequence of SEQ ID NO: 24, an LCDR2 having the amino acid sequence of SEQ ID NO: 5, and an LCDR3 having the amino acid sequence of SEQ ID NO: 6.

5. The anti-LAYN antibody, or an antigen-binding fragment thereof, according to any one of the previous claims, wherein the antibody or the antigen-binding fragment comprises: a heavy chain variable region comprising an amino acid sequence at least 80% (preferably at least 85%, more preferably at least 90%, even more preferably at least 95%) identical to the amino acid sequence of any one of SEQ ID NOs 7, 13, 34 or 143; and a light chain variable region comprising an amino acid sequence at least 80% (preferably at least 85%, more preferably at least 90%, even more preferably at least 95%) identical to the amino acid sequence of any one of SEQ ID NOs 8, 14 or 79.

6. The anti-LAYN antibody, or an antigen-binding fragment thereof, according to any one of the previous claims, wherein the antibody or the antigen-binding fragment comprises:
(i) a heavy chain variable region comprising an amino acid sequence at least 80% (preferably at least 85%, more preferably at least 90%, even more preferably at least 95%) identical to the amino acid sequence of SEQ ID NO: 7, and a light chain variable region comprising an amino acid sequence at least 80% (preferably at least 85%, more preferably at least 90%, even more preferably at least 95%) identical to the amino acid sequence of SEQ ID NO: 8; or
(ii) a heavy chain variable region comprising an amino acid sequence at least 80% (preferably at least 85%, more preferably at least 90%, even more preferably at least 95%) identical to the amino acid sequence of SEQ ID NO: 13, and a light chain variable region comprising an amino acid sequence at least 80% (preferably at least 85%, more preferably at least 90%, even more preferably at least 95%) identical to the amino acid sequence of SEQ ID NO: 14; or
(iii) a heavy chain variable region comprising an amino acid sequence at least 80% (preferably at least 85%, more preferably at least 90%, even more preferably at least 95%) identical to the amino acid sequence of SEQ ID NO: 143, and a light chain variable region comprising an amino acid sequence at least 80% (preferably at least 85%, more preferably at least 90%, even more preferably at least 95%) identical to the amino acid sequence of SEQ ID NO: 79; or
(iv) a heavy chain variable region comprising an amino acid sequence at least 80% (preferably at least 85%, more preferably at least 90%, even more preferably at least 95%) identical to the amino acid sequence of SEQ ID NO: 34, and a light chain variable region comprising an amino acid sequence at least 80% (preferably at least 85%, more preferably at least 90%, even more preferably at least 95%) identical to the amino acid sequence of SEQ ID NO: 79.

7. The anti-LAYN antibody, or an antigen-binding fragment thereof, according to any one of the previous claims, wherein the antibody or the antigen-binding fragment comprises:
(i) a heavy chain variable region comprising the amino acid sequence of SEQ ID NO: 7, and a light chain variable region comprising the amino acid sequence of SEQ ID NO: 8; or
(ii) a heavy chain variable region comprising the amino acid sequence of SEQ ID NO: 13, and a light chain variable region comprising the amino acid sequence of SEQ ID NO: 14; or
(iii) a heavy chain variable region comprising the amino acid sequence of SEQ ID NO: 143, and a light chain variable region comprising the amino acid sequence of SEQ ID NO: 79; or
(iv) a heavy chain variable region comprising the amino acid sequence of SEQ ID NO: 34, and a light chain variable region comprising the amino acid sequence of SEQ ID NO: 79.

8. The anti-LAYN antibody, or an antigen-binding fragment thereof, according to any one of the previous claims, wherein the antibody or the antigen-binding fragment comprises:
(i) a heavy chain comprising an amino acid sequence at least 80% (preferably at least 85%, more preferably at least 90%, even more preferably at least 95%) identical to the amino acid sequence of SEQ ID NO: 9, and a light chain comprising an amino acid sequence at least 80% (preferably at least 85%, more preferably at least 90%, even more preferably at least 95%) identical to the amino acid sequence of SEQ ID NO: 10; or
(ii) a heavy chain comprising an amino acid sequence at least 80% (preferably at least 85%, more preferably at least 90%, even more preferably at least 95%) identical to the amino acid sequence of SEQ ID NO: 15, and a light chain comprising an amino acid sequence at least 80% (preferably at least 85%, more preferably at least 90%, even more preferably at least 95%) identical to the amino acid sequence of SEQ ID NO: 16; or
(iii) a heavy chain comprising an amino acid sequence at least 80% (preferably at least 85%, more preferably at least 90%, even more preferably at least 95%) identical to the amino acid sequence of SEQ ID NO: 144, and a light chain comprising an amino acid sequence at least 80% (preferably at least 85%, more preferably at least 90%, even more preferably at least 95%) identical to the amino acid sequence of SEQ ID NO: 81; or
(iv) a heavy chain comprising an amino acid sequence at least 80% (preferably at least 85%, more preferably at least 90%, even more preferably at least 95%) identical to the amino acid sequence of SEQ ID NO: 36, and a light chain comprising an amino acid sequence at least 80% (preferably at least 85%, more preferably at least 90%, even more preferably at least 95%) identical to the amino acid sequence of SEQ ID NO: 81.

9. The anti-LAYN antibody, or an antigen-binding fragment thereof, according to any one of the previous claims, wherein the antibody or the antigen-binding fragment comprises:
(i) a heavy chain comprising the amino acid sequence of SEQ ID NO: 9, and a light chain comprising the amino acid sequence of SEQ ID NO: 10; or
(ii) a heavy chain comprising the amino acid sequence of SEQ ID NO: 15, and a light chain comprising the amino acid sequence of SEQ ID NO: 16; or
(iii) a heavy chain comprising the amino acid sequence of SEQ ID NO: 144, and a light chain comprising the amino acid sequence of SEQ ID NO: 81; or
(iv) a heavy chain comprising the amino acid sequence of SEQ ID NO: 36, and a light chain comprising the amino acid sequence of SEQ ID NO: 81.

10. The anti-LAYN antibody, or an antigen-binding fragment thereof, according to any one of the previous claims, wherein the antibody or the antigen-binding fragment comprises an Fc region, preferably wherein said antibody comprises an IgG₁ heavy chain constant region and/or wherein said antibody comprises a heavy chain comprising an amino acid sequence at least 95% identical to the amino acid sequence of SEQ ID NO: 173; and a light chain comprising an amino acid sequence at least 95% identical to the amino acid sequence of SEQ ID NO: 174.

11. The anti-LAYN antibody or antigen-binding fragment thereof according to any one of the previous claims, wherein the anti-LAYN antibody or the antigen-binding fragment thereof:
(a) comprises a heavy chain constant region wherein the heavy chain constant region is of a human IgG1 with (i) G236A/A330L/1332E or (ii) S239D/1332E substitutions according to the EU-index of Kabat; and/or
(b) is afucosylated.

12. A pharmaceutical composition comprising the antibody, or an antigen-binding fragment thereof, according to any one of the previous claims and, optionally, a pharmaceutically acceptable carrier.

13. The anti-LAYN antibody, or an antigen-binding fragment thereof, according to any one of claims 1-11 or the pharmaceutical composition of claim 12 for use as a medicament.

14. The anti-LAYN antibody, or an antigen-binding fragment thereof, according to any one of claims 1-11 or the pharmaceutical composition of claim 12 for use in the treatment of cancer, such as a solid tumor, optionally wherein the cancer is selected from the group consisting of non-small cell lung cancer (NSCLC), head and neck squamous cell carcinoma (HNSCC), colorectal cancer (CRC), ovarian cancer (OC), breast cancer, renal cell carcinoma (RCC), hepatocellular carcinoma (HCC), gastric cancer, bladder cancer, pancreatic cancer and melanoma.

15. The anti-LAYN antibody, or an antigen-binding fragment thereof, or the pharmaceutical composition for use according to claim 13 or 14, wherein the anti-LAYN antibody, or the fragment thereof, or the pharmaceutical composition is administered in combination with an immune checkpoint inhibitor, preferably a PD-1 antagonist.

16. A polynucleotide or a plurality of polynucleotides encoding a sequence comprising the heavy chain variable region and/or the light chain variable region of the antibody, or an antigen-binding fragment thereof, according to any one of claims 1-11.

17. A vector or a plurality of vectors comprising the polynucleotide or the plurality of polynucleotides according to claim 16.

18. A host cell comprising the polynucleotide or the plurality of polynucleotides according to claim 16; or the vector or the plurality of vectors according to claim 17.


SEQUENCE LISTING (272 sequences)

SEQ ID NO: 1 - protein, 5 aa, Mus musculus
SYWMD

SEQ ID NO: 2 - protein, 17 aa, synthetic construct
NIYPSSGTTNYAQKFQG

SEQ ID NO: 3 - protein, 10 aa, synthetic construct
ERYGRNAAEY

SEQ ID NO: 4 - protein, 11 aa, synthetic construct
RASQGIGSWLA

SEQ ID NO: 5 - protein, 7 aa, Mus musculus
AATSLAD

SEQ ID NO: 6 - protein, 9 aa, Mus musculus
QQLYSTPLT

SEQ ID NO: 7 - protein, 119 aa, synthetic construct
QVQLVQSGAEVKKPGASVKVSCKASGYTFTSYWMDWVRQAPGQGLEWMGNIYPSSGTTNYAQKFQGRVTMTVDKSTSTAYMELSSLRSEDTAVYYCARERYGRNAAEYWGQGTLVTVSS

SEQ ID NO: 8 - protein, 107 aa, synthetic construct
DIQMTQSPSSVSASVGDRVTITCRASQGIGSWLAWYQQKPGKAPKLLIYAATSLADGVPSRFSGSGSGTDFTLTISSLQPEDFATYYCQQLYSTPLTFGGGTKLEIK

SEQ ID NO: 9 - protein, 448 aa, synthetic construct
QVQLVQSGAEVKKPGASVKVSCKASGYTFTSYWMDWVRQAPGQGLEWMGNIYPSSGTTNYAQKFQGRVTMTVDKSTSTAYMELSSLRSEDTAVYYCARERYGRNAAEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
DIQMTQSPSSVSASVGDRVTITCRASQGIGSWLAWYQQKPGKAPKLLIYAATSLADGVPSRFSGSGSGTDFTLTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 17 aa, synthetic construct
NIYPGSGTTNYAQKFQG

SEQ ID NO: 12 - protein, 11 aa, synthetic construct
RASQTIGTWLA

SEQ ID NO: 13 - protein, 119 aa, synthetic construct
QVQLVQSGSEVKKPGASVKVSCKASGYTFTSYWMDWVRQAPGQGLEWMGNIYPGSGTTNYAQKFQGRATMTVDKSTSTAYMELSSLRSEDTAVYYCARERYGRNAAEYWGQGTLVTVSS

SEQ ID NO: 14 - protein, 107 aa, synthetic construct
DIQMTQSPSSVSASVGDRVTITCRASQTIGTWLAWYQQKPGKAPKLLIYAATSLADGVPSRFSGSGSGTDFTLTISSLQPEDFATYYCQQLYSTPLTFGGGTKLEIK

SEQ ID NO: 15 - protein, 448 aa, synthetic construct
QVQLVQSGSEVKKPGASVKVSCKASGYTFTSYWMDWVRQAPGQGLEWMGNIYPGSGTTNYAQKFQGRATMTVDKSTSTAYMELSSLRSEDTAVYYCARERYGRNAAEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 214 aa, synthetic construct
DIQMTQSPSSVSASVGDRVTITCRASQTIGTWLAWYQQKPGKAPKLLIYAATSLADGVPSRFSGSGSGTDFTLTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 119 aa, synthetic construct
QVQLVQSGAEVKKPGASVKVSCKASGYTFTSYWMDWVRQAPGQGLEWMGNIYPSSGTTNYAQKFQGRVTMTVDTSTSTAYMELSSLRSEDTAVYYCARERYGRNAAEYWGQGTLVTVSS

SEQ ID NO: 18 - protein, 448 aa, synthetic construct
QVQLVQSGAEVKKPGASVKVSCKASGYTFTSYWMDWVRQAPGQGLEWMGNIYPSSGTTNYAQKFQGRVTMTVDTSTSTAYMELSSLRSEDTAVYYCARERYGRNAAEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 17 aa, synthetic construct
NIYPGSGSTNYAQKFQG

SEQ ID NO: 20 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCARERYGRNAAEYWGQGTLVTVSS

SEQ ID NO: 21 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCARERYGRNAAEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 119 aa, synthetic construct
QVQLVQSGSEVKKPGASVKVSCKASGYTFTSYWMDWVRQAPGQGLEWMGNIYPGSGSTNYAQKFQGRATMTVDKSTSTAYMELSSLRSEDTAVYYCARERYGRNAAEYWGQGTLVTVSS

SEQ ID NO: 23 - protein, 448 aa, synthetic construct
QVQLVQSGSEVKKPGASVKVSCKASGYTFTSYWMDWVRQAPGQGLEWMGNIYPGSGSTNYAQKFQGRATMTVDKSTSTAYMELSSLRSEDTAVYYCARERYGRNAAEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 11 aa, Mus musculus
LASQTIGTWLA

SEQ ID NO: 25 - protein, 107 aa, synthetic construct
DIQMTQSPSSVSASVGDRVTITCLASQTIGTWLAWYQQKPGKSPKLLIYAATSLADGVPSRFSGSGSGTDFTLTISSLQPEDFATYYCQQLYSTPLTFGGGTKLEIK

SEQ ID NO: 26 - protein, 214 aa, synthetic construct
DIQMTQSPSSVSASVGDRVTITCLASQTIGTWLAWYQQKPGKSPKLLIYAATSLADGVPSRFSGSGSGTDFTLTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAAEYWGQGTLVTVSS

SEQ ID NO: 28 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAAEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 10 aa, synthetic construct
EKYGRNAMEY

SEQ ID NO: 30 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTREKYGRNAMEYWGQGTLVTVSS

SEQ ID NO: 31 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTREKYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 10 aa, synthetic construct
ERYGRNAMEY

SEQ ID NO: 33 - protein, 9 aa, synthetic construct
QQLYSFPLT

SEQ ID NO: 34 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMEYWGQGTLVTVSS

SEQ ID NO: 35 - protein, 107 aa, synthetic construct
DIQMTQSPSSVSASVGDRVTITCLASQTIGTWLAWYQQKPGKSPKLLIYAATSLADGVPSRFSGSGSGTDFTFTISSLQPEDFATYYCQQLYSFPLTFGGGTKLEIK

SEQ ID NO: 36 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 214 aa, synthetic construct
DIQMTQSPSSVSASVGDRVTITCLASQTIGTWLAWYQQKPGKSPKLLIYAATSLADGVPSRFSGSGSGTDFTFTISSLQPEDFATYYCQQLYSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 9 aa, synthetic construct
QQAYSTPLT

SEQ ID NO: 39 - protein, 107 aa, synthetic construct
DIQMTQSPSSVSASVGDRVTITCLASQTIGTWLAWYQQKPGKSPKLLIYAATSLADGVPSRFSGSGSGTDFTFTISSLQPEDFATYYCQQAYSTPLTFGGGTKLEIK

SEQ ID NO: 40 - protein, 214 aa, synthetic construct
DIQMTQSPSSVSASVGDRVTITCLASQTIGTWLAWYQQKPGKSPKLLIYAATSLADGVPSRFSGSGSGTDFTFTISSLQPEDFATYYCQQAYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 9 aa, synthetic construct
QQSYSTPLT

SEQ ID NO: 42 - protein, 107 aa, synthetic construct
DIQMTQSPSSVSASVGDRVTITCLASQTIGTWLAWYQQKPGKSPKLLIYAATSLADGVPSRFSGSGSGTDFTFTISSLQPEDFATYYCQQSYSTPLTFGGGTKLEIK

SEQ ID NO: 43 - protein, 214 aa, synthetic construct
DIQMTQSPSSVSASVGDRVTITCLASQTIGTWLAWYQQKPGKSPKLLIYAATSLADGVPSRFSGSGSGTDFTFTISSLQPEDFATYYCQQSYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 7 aa, synthetic construct
AATSLAT

SEQ ID NO: 45 - protein, 107 aa, synthetic construct
DIQMTQSPSSVSASVGDRVTITCLASQTIGTWLAWYQQKPGKSPKLLIYAATSLATGVPSRFSGSGSGTDFTFTISSLQPEDFATYYCQQLYSTPLTFGGGTKLEIK

SEQ ID NO: 46 - protein, 214 aa, synthetic construct
DIQMTQSPSSVSASVGDRVTITCLASQTIGTWLAWYQQKPGKSPKLLIYAATSLATGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 7 aa, synthetic construct
AATSLAS

SEQ ID NO: 48 - protein, 107 aa, synthetic construct
DIQMTQSPSSVSASVGDRVTITCLASQTIGTWLAWYQQKPGKSPKLLIYAATSLASGVPSRFSGSGSGTDFTFTISSLQPEDFATYYCQQLYSTPLTFGGGTKLEIK

SEQ ID NO: 49 - protein, 214 aa, synthetic construct
DIQMTQSPSSVSASVGDRVTITCLASQTIGTWLAWYQQKPGKSPKLLIYAATSLASGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 7 aa, synthetic construct
AATSLQD

SEQ ID NO: 51 - protein, 107 aa, synthetic construct
DIQMTQSPSSVSASVGDRVTITCLASQTIGTWLAWYQQKPGKSPKLLIYAATSLQDGVPSRFSGSGSGTDFTFTISSLQPEDFATYYCQQLYSTPLTFGGGTKLEIK

SEQ ID NO: 52 - protein, 214 aa, synthetic construct
DIQMTQSPSSVSASVGDRVTITCLASQTIGTWLAWYQQKPGKSPKLLIYAATSLQDGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 7 aa, synthetic construct
AASSLAD

SEQ ID NO: 54 - protein, 107 aa, synthetic construct
DIQMTQSPSSVSASVGDRVTITCLASQTIGTWLAWYQQKPGKSPKLLIYAASSLADGVPSRFSGSGSGTDFTFTISSLQPEDFATYYCQQLYSTPLTFGGGTKLEIK

SEQ ID NO: 55 - protein, 214 aa, synthetic construct
DIQMTQSPSSVSASVGDRVTITCLASQTIGTWLAWYQQKPGKSPKLLIYAASSLADGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 107 aa, synthetic construct
DIQMTQSPSSVSASVGDRVTITCLASQTIGTWLAWYQQKPGKAPKLLIYAATSLADGVPSRFSGSGSGTDFTFTISSLQPEDFATYYCQQLYSTPLTFGGGTKLEIK

SEQ ID NO: 57 - protein, 214 aa, synthetic construct
DIQMTQSPSSVSASVGDRVTITCLASQTIGTWLAWYQQKPGKAPKLLIYAATSLADGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 11 aa, synthetic construct
LASQTIGTYLA

SEQ ID NO: 59 - protein, 107 aa, synthetic construct
DIQMTQSPSSVSASVGDRVTITCLASQTIGTYLAWYQQKPGKSPKLLIYAATSLADGVPSRFSGSGSGTDFTFTISSLQPEDFATYYCQQLYSTPLTFGGGTKLEIK

SEQ ID NO: 60 - protein, 214 aa, synthetic construct
DIQMTQSPSSVSASVGDRVTITCLASQTIGTYLAWYQQKPGKSPKLLIYAATSLADGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 11 aa, synthetic construct
LASQTIGSWLA

SEQ ID NO: 62 - protein, 107 aa, synthetic construct
DIQMTQSPSSVSASVGDRVTITCLASQTIGSWLAWYQQKPGKSPKLLIYAATSLADGVPSRFSGSGSGTDFTFTISSLQPEDFATYYCQQLYSTPLTFGGGTKLEIK

SEQ ID NO: 63 - protein, 214 aa, synthetic construct
DIQMTQSPSSVSASVGDRVTITCLASQTIGSWLAWYQQKPGKSPKLLIYAATSLADGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 11 aa, synthetic construct
LASQTISTWLA

SEQ ID NO: 65 - protein, 107 aa, synthetic construct
DIQMTQSPSSVSASVGDRVTITCLASQTISTWLAWYQQKPGKSPKLLIYAATSLADGVPSRFSGSGSGTDFTFTISSLQPEDFATYYCQQLYSTPLTFGGGTKLEIK

SEQ ID NO: 66 - protein, 214 aa, synthetic construct
DIQMTQSPSSVSASVGDRVTITCLASQTISTWLAWYQQKPGKSPKLLIYAATSLADGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 11 aa, synthetic construct
LASQGIGTWLA

SEQ ID NO: 68 - protein, 107 aa, synthetic construct
DIQMTQSPSSVSASVGDRVTITCLASQGIGTWLAWYQQKPGKSPKLLIYAATSLADGVPSRFSGSGSGTDFTFTISSLQPEDFATYYCQQLYSTPLTFGGGTKLEIK

SEQ ID NO: 69 - protein, 214 aa, synthetic construct
DIQMTQSPSSVSASVGDRVTITCLASQGIGTWLAWYQQKPGKSPKLLIYAATSLADGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 11 aa, synthetic construct
LASQSIGTWLA

SEQ ID NO: 71 - protein, 107 aa, synthetic construct
DIQMTQSPSSVSASVGDRVTITCLASQSIGTWLAWYQQKPGKSPKLLIYAATSLADGVPSRFSGSGSGTDFTFTISSLQPEDFATYYCQQLYSTPLTFGGGTKLEIK

SEQ ID NO: 72 - protein, 214 aa, synthetic construct
DIQMTQSPSSVSASVGDRVTITCLASQSIGTWLAWYQQKPGKSPKLLIYAATSLADGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 107 aa, synthetic construct
DIQMTQSPSSVSASVGDRVTITCRASQTIGTWLAWYQQKPGKSPKLLIYAATSLADGVPSRFSGSGSGTDFTFTISSLQPEDFATYYCQQLYSTPLTFGGGTKLEIK

SEQ ID NO: 74 - protein, 214 aa, synthetic construct
DIQMTQSPSSVSASVGDRVTITCRASQTIGTWLAWYQQKPGKSPKLLIYAATSLADGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 107 aa, synthetic construct
DIQMTQSPSSLSASVGDRVTITCLASQTIGTWLAWYQQKPGKSPKLLIYAATSLADGVPSRFSGSGSGTDFTFTISSLQPEDFATYYCQQLYSTPLTFGGGTKLEIK

SEQ ID NO: 76 - protein, 214 aa, synthetic construct
DIQMTQSPSSLSASVGDRVTITCLASQTIGTWLAWYQQKPGKSPKLLIYAATSLADGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 10 aa, synthetic construct
ERYGRNAMEA

SEQ ID NO: 78 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMEAWGQGTLVTVSS

SEQ ID NO: 79 - protein, 107 aa, synthetic construct
DIQMTQSPSSVSASVGDRVTITCLASQTIGTWLAWYQQKPGKSPKLLIYAATSLADGVPSRFSGSGSGTDFTFTISSLQPEDFATYYCQQLYSTPLTFGGGTKLEIK

SEQ ID NO: 80 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMEAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 214 aa, synthetic construct
DIQMTQSPSSVSASVGDRVTITCLASQTIGTWLAWYQQKPGKSPKLLIYAATSLADGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 10 aa, synthetic construct
ERYGRNAMAY

SEQ ID NO: 83 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMAYWGQGTLVTVSS

SEQ ID NO: 84 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 10 aa, synthetic construct
ERYGRAAMEY

SEQ ID NO: 86 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRAAMEYWGQGTLVTVSS

SEQ ID NO: 87 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRAAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 10 aa, synthetic construct
ERYARNAMEY

SEQ ID NO: 89 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYARNAMEYWGQGTLVTVSS

SEQ ID NO: 90 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYARNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 10 aa, synthetic construct
EAYGRNAMEY

SEQ ID NO: 92 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTREAYGRNAMEYWGQGTLVTVSS

SEQ ID NO: 93 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTREAYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 10 aa, synthetic construct
ARYGRNAMEY

SEQ ID NO: 95 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRARYGRNAMEYWGQGTLVTVSS

SEQ ID NO: 96 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRARYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCARERYGRNAMEYWGQGTLVTVSS

SEQ ID NO: 98 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCARERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSDDTAVYYCTRERYGRNAMEYWGQGTLVTVSS

SEQ ID NO: 100 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSDDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSRLRSEDTAVYYCTRERYGRNAMEYWGQGTLVTVSS

SEQ ID NO: 102 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSRLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTLYMELSSLRSEDTAVYYCTRERYGRNAMEYWGQGTLVTVSS

SEQ ID NO: 104 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTLYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTVYMELSSLRSEDTAVYYCTRERYGRNAMEYWGQGTLVTVSS

SEQ ID NO: 106 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTVYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSKSTAYMELSSLRSEDTAVYYCTRERYGRNAMEYWGQGTLVTVSS

SEQ ID NO: 108 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSKSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSISTAYMELSSLRSEDTAVYYCTRERYGRNAMEYWGQGTLVTVSS

SEQ ID NO: 110 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSISTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 119 aa, synthetic construct
QVQLVQSGSEVKKPGASVKVSCKASGYTFTSYWMDWVRQAPGQGLEWMGNIYPGSGSTNYAQKFQGRATLTVDTSTSTAYMELSSLRSEDTAVYYCTRERYGRNAMEYWGQGTLVTVSS

SEQ ID NO: 112 - protein, 448 aa, synthetic construct
QVQLVQSGSEVKKPGASVKVSCKASGYTFTSYWMDWVRQAPGQGLEWMGNIYPGSGSTNYAQKFQGRATLTVDTSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 119 aa, synthetic construct
QVQLVQSGSEVKKPGASVKVSCKASGYTFTSYWMDWVRQAPGQGLEWMGNIYPGSGSTNYAQKFQGRATLTADKSTSTAYMELSSLRSEDTAVYYCTRERYGRNAMEYWGQGTLVTVSS

SEQ ID NO: 114 - protein, 448 aa, synthetic construct
QVQLVQSGSEVKKPGASVKVSCKASGYTFTSYWMDWVRQAPGQGLEWMGNIYPGSGSTNYAQKFQGRATLTADKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 119 aa, synthetic construct
QVQLVQSGSEVKKPGASVKVSCKASGYTFTSYWMDWVRQAPGQGLEWMGNIYPGSGSTNYAQKFQGRATLTRDKSTSTAYMELSSLRSEDTAVYYCTRERYGRNAMEYWGQGTLVTVSS

SEQ ID NO: 116 - protein, 448 aa, synthetic construct
QVQLVQSGSEVKKPGASVKVSCKASGYTFTSYWMDWVRQAPGQGLEWMGNIYPGSGSTNYAQKFQGRATLTRDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 119 aa, synthetic construct
QVQLVQSGSEVKKPGASVKVSCKASGYTFTSYWMDWVRQAPGQGLEWMGNIYPGSGSTNYAQKFQGRATITVDKSTSTAYMELSSLRSEDTAVYYCTRERYGRNAMEYWGQGTLVTVSS

SEQ ID NO: 118 - protein, 448 aa, synthetic construct
QVQLVQSGSEVKKPGASVKVSCKASGYTFTSYWMDWVRQAPGQGLEWMGNIYPGSGSTNYAQKFQGRATITVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 119 aa, synthetic construct
QVQLVQSGSEVKKPGASVKVSCKASGYTFTSYWMDWVRQAPGQGLEWMGNIYPGSGSTNYAQKFQGRATMTVDKSTSTAYMELSSLRSEDTAVYYCTRERYGRNAMEYWGQGTLVTVSS

SEQ ID NO: 120 - protein, 448 aa, synthetic construct
QVQLVQSGSEVKKPGASVKVSCKASGYTFTSYWMDWVRQAPGQGLEWMGNIYPGSGSTNYAQKFQGRATMTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 119 aa, synthetic construct
QVQLVQSGSEVKKPGASVKVSCKASGYTFTSYWMDWVRQAPGQGLEWMGNIYPGSGSTNYAQKFQGRVTLTVDKSTSTAYMELSSLRSEDTAVYYCTRERYGRNAMEYWGQGTLVTVSS

SEQ ID NO: 122 - protein, 448 aa, synthetic construct
QVQLVQSGSEVKKPGASVKVSCKASGYTFTSYWMDWVRQAPGQGLEWMGNIYPGSGSTNYAQKFQGRVTLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 119 aa, synthetic construct
QVQLVQSGSEVKKPGASVKVSCKASGYTFTSYWMDWVRQAPGQGLEWMGNIYPGSGTTNYAQKFQGRATLTVDKSTSTAYMELSSLRSEDTAVYYCTRERYGRNAMEYWGQGTLVTVSS

SEQ ID NO: 124 - protein, 448 aa, synthetic construct
QVQLVQSGSEVKKPGASVKVSCKASGYTFTSYWMDWVRQAPGQGLEWMGNIYPGSGTTNYAQKFQGRATLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 125 - protein, 17 aa, synthetic construct
NIYPSSGSTNYAQKFQG

SEQ ID NO: 126 - protein, 119 aa, synthetic construct
QVQLVQSGSEVKKPGASVKVSCKASGYTFTSYWMDWVRQAPGQGLEWMGNIYPSSGSTNYAQKFQGRATLTVDKSTSTAYMELSSLRSEDTAVYYCTRERYGRNAMEYWGQGTLVTVSS

SEQ ID NO: 127 - protein, 448 aa, synthetic construct
QVQLVQSGSEVKKPGASVKVSCKASGYTFTSYWMDWVRQAPGQGLEWMGNIYPSSGSTNYAQKFQGRATLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 17 aa, synthetic construct
NINPGSGSTNYAQKFQG

SEQ ID NO: 129 - protein, 119 aa, synthetic construct
QVQLVQSGSEVKKPGASVKVSCKASGYTFTSYWMDWVRQAPGQGLEWMGNINPGSGSTNYAQKFQGRATLTVDKSTSTAYMELSSLRSEDTAVYYCTRERYGRNAMEYWGQGTLVTVSS

SEQ ID NO: 130 - protein, 448 aa, synthetic construct
QVQLVQSGSEVKKPGASVKVSCKASGYTFTSYWMDWVRQAPGQGLEWMGNINPGSGSTNYAQKFQGRATLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 5 aa, synthetic construct
GYWMD

SEQ ID NO: 132 - protein, 119 aa, synthetic construct
QVQLVQSGSEVKKPGASVKVSCKASGYTFTGYWMDWVRQAPGQGLEWMGNIYPGSGSTNYAQKFQGRATLTVDKSTSTAYMELSSLRSEDTAVYYCTRERYGRNAMEYWGQGTLVTVSS

SEQ ID NO: 133 - protein, 448 aa, synthetic construct
QVQLVQSGSEVKKPGASVKVSCKASGYTFTGYWMDWVRQAPGQGLEWMGNIYPGSGSTNYAQKFQGRATLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - protein, 119 aa, synthetic construct
QVQLVQSGTEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMEYWGQGTLVTVSS

SEQ ID NO: 135 - protein, 448 aa, synthetic construct
QVQLVQSGTEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 136 - protein, 119 aa, synthetic construct
QVQLVQSGAEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMEYWGQGTLVTVSS

SEQ ID NO: 137 - protein, 448 aa, synthetic construct
QVQLVQSGAEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - protein, 107 aa, synthetic construct
DIQMTQSPSSVSASVGDRVTITCRASQTIGTWLAWYQQKPGKSPKLLIYAATSLADGVPSRFSGSGSGTDFTLTISSLQPEDFATYYCQQLYSTPLTFGGGTKLEIK

SEQ ID NO: 139 - protein, 214 aa, synthetic construct
DIQMTQSPSSVSASVGDRVTITCRASQTIGTWLAWYQQKPGKSPKLLIYAATSLADGVPSRFSGSGSGTDFTLTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 119 aa, synthetic construct
QVQLVQSGAEVKKPGASVKVSCKASGYTFTSYWMDWVRQAPGQGLEWMGNIYPSSGSTNYAQKFQGRVTMTVDTSTSTVYMELSSLRSEDTAVYYCARERYGRNAAEYWGQGTLVTVSS

SEQ ID NO: 141 - protein, 448 aa, synthetic construct
QVQLVQSGAEVKKPGASVKVSCKASGYTFTSYWMDWVRQAPGQGLEWMGNIYPSSGSTNYAQKFQGRVTMTVDTSTSTVYMELSSLRSEDTAVYYCARERYGRNAAEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 142 - protein, 10 aa, Mus musculus
ERYGRNAMDY

SEQ ID NO: 143 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMDYWGQGTLVTVSS

SEQ ID NO: 144 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 145 - protein, 119 aa, synthetic construct
QVQLVQSGSEVKKPGASVKVSCKASGYTFTSYWMDWVRQAPGKGLEWMGNIYPGSGSTNYAQKFQGRVTLTVDKATSTAYMELSSLRSEDTATYYCTRERYGRNAMDYWGQGTLVTVSS

SEQ ID NO: 146 - protein, 448 aa, synthetic construct
QVQLVQSGSEVKKPGASVKVSCKASGYTFTSYWMDWVRQAPGKGLEWMGNIYPGSGSTNYAQKFQGRVTLTVDKATSTAYMELSSLRSEDTATYYCTRERYGRNAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 147 - protein, 9 aa, synthetic construct
QQYYSTPLT

SEQ ID NO: 148 - protein, 107 aa, synthetic construct
DIQMTQSPSSVSASVGDRVTITCLASQTIGTWLAWYQQKPGKSPKLLIYAATSLADGVPSRFSGSGSGTDFTFTISSLQPEDFATYYCQQYYSTPLTFGGGTKLEIK

SEQ ID NO: 149 - protein, 214 aa, synthetic construct
DIQMTQSPSSVSASVGDRVTITCLASQTIGTWLAWYQQKPGKSPKLLIYAATSLADGVPSRFSGSGSGTDFTFTISSLQPEDFATYYCQQYYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 10 aa, synthetic construct
ERYGANAMEY

SEQ ID NO: 151 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGANAMEYWGQGTLVTVSS

SEQ ID NO: 152 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGANAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 153 - protein, 10 aa, synthetic construct
ERAGRNAMEY

SEQ ID NO: 154 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERAGRNAMEYWGQGTLVTVSS

SEQ ID NO: 155 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERAGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 17 aa, synthetic construct
WIYPGSGSTNYAQKFQG

SEQ ID NO: 157 - protein, 119 aa, synthetic construct
QVQLVQSGSEVKKPGASVKVSCKASGYTFTSYWMDWVRQAPGQGLEWMGWIYPGSGSTNYAQKFQGRATLTVDKSTSTAYMELSSLRSEDTAVYYCTRERYGRNAMEYWGQGTLVTVSS

SEQ ID NO: 158 - protein, 448 aa, synthetic construct
QVQLVQSGSEVKKPGASVKVSCKASGYTFTSYWMDWVRQAPGQGLEWMGWIYPGSGSTNYAQKFQGRATLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 159 - protein, 17 aa, synthetic construct
IIYPGSGSTNYAQKFQG

SEQ ID NO: 160 - protein, 119 aa, synthetic construct
QVQLVQSGSEVKKPGASVKVSCKASGYTFTSYWMDWVRQAPGQGLEWMGIIYPGSGSTNYAQKFQGRATLTVDKSTSTAYMELSSLRSEDTAVYYCTRERYGRNAMEYWGQGTLVTVSS

SEQ ID NO: 161 - protein, 448 aa, synthetic construct
QVQLVQSGSEVKKPGASVKVSCKASGYTFTSYWMDWVRQAPGQGLEWMGIIYPGSGSTNYAQKFQGRATLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - protein, 5 aa, synthetic construct
SYWMH

SEQ ID NO: 163 - protein, 119 aa, synthetic construct
QVQLVQSGSEVKKPGASVKVSCKASGYTFTSYWMHWVRQAPGQGLEWMGNIYPGSGSTNYAQKFQGRATLTVDKSTSTAYMELSSLRSEDTAVYYCTRERYGRNAMEYWGQGTLVTVSS

SEQ ID NO: 164 - protein, 448 aa, synthetic construct
QVQLVQSGSEVKKPGASVKVSCKASGYTFTSYWMHWVRQAPGQGLEWMGNIYPGSGSTNYAQKFQGRATLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 5 aa, synthetic construct
SYYMD

SEQ ID NO: 166 - protein, 119 aa, synthetic construct
QVQLVQSGSEVKKPGASVKVSCKASGYTFTSYYMDWVRQAPGQGLEWMGNIYPGSGSTNYAQKFQGRATLTVDKSTSTAYMELSSLRSEDTAVYYCTRERYGRNAMEYWGQGTLVTVSS

SEQ ID NO: 167 - protein, 448 aa, synthetic construct
QVQLVQSGSEVKKPGASVKVSCKASGYTFTSYYMDWVRQAPGQGLEWMGNIYPGSGSTNYAQKFQGRATLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 17 aa, Mus musculus
NIYPGSGSTNYDEKFKS

SEQ ID NO: 169 - protein, 119 aa, Mus musculus
QVQLQQPGSELVRPGASVKLSCKASGYTFTSYWMDWVKQRPGQGLEWIGNIYPGSGSTNYDEKFKSKATLTVDKSSSTAYMQLSSLTSEDSAVYYCTRERYGRNAMDYWGQGTSVTVSS

SEQ ID NO: 170 - protein, 107 aa, Mus musculus
DIQMTQSPASQSASLGESVTITCLASQTIGTWLAWYQQKPGKSPQLLIYAATSLADGVPSRFSGSGSGTKFSFKISSLQAEDFVSYYCQQLYSTPLTFGGGTKLEIK

SEQ ID NO: 171 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 172 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 174 - protein, 107 aa, Homo sapiens [sequence omitted]

SEQ ID NO: 175 - protein, 449 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 176 - protein, 214 aa, synthetic construct
DIQMTQSPSSVSASVGDRVTITCLASQTIGTWLAWYQQKPGKSPKLLIYAATSLADGVPSRFSGSGSGTDFTFTISSLQPEDFATYYCQQLYSTPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 177 - protein, 238 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRNAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKENLYFQGSHHHHHH

SEQ ID NO: 178 - protein, 7 aa, synthetic construct
AATSLAE

SEQ ID NO: 179 - protein, 107 aa, synthetic construct
DIQMTQSPSSVSASVGDRVTITCLASQTIGTWLAWYQQKPGKSPKLLIYAATSLAEGVPSRFSGSGSGTDFTFTISSLQPEDFATYYCQQLYSTPLTFGGGTKLEIK

SEQ ID NO: 180 - protein, 214 aa, synthetic construct
DIQMTQSPSSVSASVGDRVTITCLASQTIGTWLAWYQQKPGKSPKLLIYAATSLAEGVPSRFSGSGSGTDFTFTISSLQPEDFATYYCQQLYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 119 aa, synthetic construct
QVQLVQSGSEVKKPGASVKVSCKASGYTFTSYWMDWVRQAPGKGLEWMGNIYPGSGSTNYAQKFQGRVTLTVDKSTDTAYMELSSLRSEDTATYYCTRERYGRNAMDYWGQGTLVTVSS

SEQ ID NO: 182 - protein, 448 aa, synthetic construct
QVQLVQSGSEVKKPGASVKVSCKASGYTFTSYWMDWVRQAPGKGLEWMGNIYPGSGSTNYAQKFQGRVTLTVDKSTDTAYMELSSLRSEDTATYYCTRERYGRNAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 183 - protein, 10 aa, synthetic construct
ERYGRQAMDY

SEQ ID NO: 184 - protein, 119 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRQAMDYWGQGTLVTVSS

SEQ ID NO: 185 - protein, 448 aa, synthetic construct
QVQLVQSGSEVKKPGASVKVSCKASGYTFTSYWMDWVRQAPGQGLEWMGNIYPGSGSTNYAQKFQGRATLTVDKSTSTAYMELSSLRSEDTAVYYCTRERYGRQAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 186 - protein, 119 aa, synthetic construct
QVQLVQSGSEVKKPGASVKVSCKASGYTFTSYWMDWVRQAPGKGLEWMGNIYPGSGSTNYAQKFQGRVTLTVDKATSTAYMELSSLRSEDTATYYCTRERYGRQAMDYWGQGTLVTVSS

SEQ ID NO: 187 - protein, 448 aa, synthetic construct
QVQLVQSGSEVKKPGASVKVSCKASGYTFTSYWMDWVRQAPGKGLEWMGNIYPGSGSTNYAQKFQGRVTLTVDKATSTAYMELSSLRSEDTATYYCTRERYGRQAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 188 - protein, 119 aa, synthetic construct
QVQLVQSGSEVKKPGASVKVSCKASGYTFTSYWMDWVRQAPGKGLEWMGNIYPGSGSTNYAQKFQGRVTLTVDKSTDTAYMELSSLRSEDTATYYCTRERYGRQAMDYWGQGTLVTVSS

SEQ ID NO: 189 - protein, 448 aa, synthetic construct
QVQLVQSGSEVKKPGASVKVSCKASGYTFTSYWMDWVRQAPGKGLEWMGNIYPGSGSTNYAQKFQGRVTLTVDKSTDTAYMELSSLRSEDTATYYCTRERYGRQAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 190 - protein, 215 aa, synthetic construct
RLLSGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYGGHHHHHHHHHH

SEQ ID NO: 191 - protein, 203 aa, Homo sapiens
RLLSGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAY

SEQ ID NO: 192 - protein, 168 aa, synthetic construct
RLLSGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDGGHHHHHHHHHH

SEQ ID NO: 193 - protein, 374 aa, Homo sapiens
MRPGTALQAVLLAVLLVGLRAATGRLLSGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 194 - protein, 350 aa, Homo sapiens
RLLSGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 195 - protein, 250 aa, Homo sapiens
MRPGTALQAVLLAVLLVGLRAATGRLLSGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWI

SEQ ID NO: 196 - protein, 223 aa, synthetic construct
RLLSASDLDLRGGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYGGHHHHHHHHHH

SEQ ID NO: 197 - protein, 211 aa, Homo sapiens
RLLSASDLDLRGGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAY

SEQ ID NO: 198 - protein, 82 aa, Homo sapiens
MVTSGLGSGGVRRNKAIAQPARTFMLGLMAAYHNLEKPAVPSREAEGEETELTTPVLPEETQEEDAKKTFKESREAALNLAY

SEQ ID NO: 199 - protein, 83 aa, Homo sapiens
MYHQPSAPAGIGGPYMFQWNDDRCNMKNNFICKYSDEKPAVPSREAEGEETELTTPVLPEETQEEDAKKTFKESREAALNLAY

SEQ ID NO: 200 - protein, 203 aa, Macaca fascicularis
RLLSGQPVCRGGTQRPCYKVIYFHDTSRRLNFEEAKEACRRDGGQLVSIESEDEQNLIEKFIENLLPSDGDFWIGLRRREEKQSNSTACEDLYAWTDGSISQFRNWYVDEPSCGSEVCVVMYHQPSAPAGIGGPYMFQWNDDRCNMKNNFICKYSDEKPAVPSGEPEGEETEPTTPVLLEETQEEDAKKTFKESREAALNLAY

SEQ ID NO: 201 - protein, 213 aa, Canis lupus
RLLSGQPVCREGTQRPCYKVIYFHDASRRLNFEEANAACRTDGGQLLSIESQDEQRLIEKFIENLLASDGDFWIGLRRREERQSNSTACQDLYAWTDGSTSAFRNWYVDEPSCGSEVCVVMYHQPSAPAGVGGLYMFQWNDDRCNMKNNFICKYSDEKPTAPSIKPGGEGTEPATPILPGDAGKEDARETFKGNNGMDRTLQGKIEAALNLAY

SEQ ID NO: 202 - protein, 200 aa, Rattus norvegicus
HLLSGQRFCWEGTRRPCYRVIYFHDTSQRRNFEEAKEACMKDGGQLASIETADEQRLIENFIESLQASDGDFWIGLKRLEEKQHNSTACQDLYAWTDGSLSQFRNWYMDEPSCESEVCVVMYHQPSAPPGIGGPYMFQWNDDRCNTKKNFICKYSDDKPSTTPSLSPGGEATEPAAPILPEETLEIDTKERREAALNLAY

SEQ ID NO: 203 - protein, 204 aa, Mus musculus
RLLSGQLVCRGGTRRPCYKVIYFHDAFQRLNFEEAKETCMEDGGQLVSIETEDEQRLIEKFIENLLASDGDFWIGLKRLEEKQSNNTACQDLYAWTDGSTSQFRNWYVDEPSCGSEVCVVMYHQPSAPPGIGGSYMFQWNDDRCNMKNNFICKYHDDKPSTTPSIWPGGEATEPATPLLPEETQKEDTKETFKERREAALNLAY

SEQ ID NO: 204 - protein, 203 aa, Oryctolagus cuniculus
RLLSGQLVCRGGTQRPCYKVIYFHDASRRLNFEEAKEACRRDSGQLVSIESEDEQRLIEKFIENLLASDGDFWIGLRRQEEKQNNRTACQDLYSWTDGSSSKFRNWYVDEPSCGSEVCVVMYHQPSAPAGIGGPYMFQWNDDRCNMKNNFICKYSDEKPTPPSTRLGGAATEPATPILPEEVQKEDAKETFKESKEAALNLAY

SEQ ID NO: 205 - protein, 203 aa, Sus scrofa
RLLSGQPVCREGTRRPCYEVIYFQESSRRLNFEEARAACGRDGGQLVSIESEDEQRLIEKFIENLHASDGDFWIGLRRWEEKQSNSTACQDLYAWTDGSTATFRNWYADEPSCGSEVCVVMYHQPSAPAGIGGRYMFQWNDDRCTMKNNFICKYSEEKPTAPPTRPGGEGTEPATPRLPEDTEKEEATGTLKESSEAALNLAY

SEQ ID NO: 206 - protein, 5 aa, synthetic construct
  VARIANT 1: note = X may be any amino acid, preferably S or G, more preferably S
XYWMD

SEQ ID NO: 207 - protein, 17 aa, synthetic construct
  VARIANT 3: note = X may be any amino acid, preferably Y or N, more preferably Y
  VARIANT 5: note = X may be any amino acid, preferably S or G, more preferably S
  VARIANT 8: note = X may be any amino acid, preferably T or S, more preferably T
  VARIANT 12: note = X may be any amino acid, preferably A or D, more preferably A
  VARIANT 13: note = X may be any amino acid, preferably Q or E, more preferably Q
  VARIANT 16: note = X may be any amino acid, preferably Q or K, more preferably Q
  VARIANT 17: note = X may be any amino acid, preferably G or S, more preferably G
NIXPXSGXTNYXXKFXX

SEQ ID NO: 208 - skipped

SEQ ID NO: 209 - protein, 11 aa, synthetic construct
  VARIANT 1: note = X may be any amino acid, preferably R or L, more preferably R
  VARIANT 5: note = X may be any amino acid, preferably G, T or S, more preferably G
  VARIANT 7: note = X may be any amino acid, preferably G or S, more preferably G
  VARIANT 8: note = X may be any amino acid, preferably S or T, more preferably S
  VARIANT 9: note = X may be any amino acid, preferably W or Y, more preferably W
XASQXIXXXLA

SEQ ID NO: 210 - protein, 7 aa, synthetic construct
  VARIANT 3: note = X may be any amino acid, preferably T or S, more preferably T
  VARIANT 6: note = X may be any amino acid, preferably A or Q, more preferably A
  VARIANT 7: note = X may be any amino acid, preferably D, T, S or E, more preferably D
AAXSLXX

SEQ ID NO: 211 - protein, 9 aa, synthetic construct
  VARIANT 3: note = X is L, A or S, preferably L
  VARIANT 6: note = X may be any amino acid, preferably T or F, more preferably T
QQXYSXPLT

SEQ ID NO: 212 - protein, 119 aa, synthetic construct
  VARIANT 5: note = X may be any amino acid, preferably V or Q, more preferably V
  VARIANT 7: note = X may be any amino acid, preferably S or P, more preferably S
  VARIANT 9: note = X may be any amino acid, preferably A, S or T, more preferably A
  VARIANT 11: note = X may be any amino acid, preferably V or L, more preferably V
  VARIANT 12: note = X may be any amino acid, preferably K or V, more preferably K
  VARIANT 13: note = X may be any amino acid, preferably K or R, more preferably K
  VARIANT 20: note = X may be any amino acid, preferably V or L, more preferably V
  VARIANT 31: note = X may be any amino acid, preferably S or G, more preferably S
  VARIANT 38: note = X may be any amino acid, preferably R or K, more preferably R
  VARIANT 40: note = X may be any amino acid, preferably A or R, more preferably A
  VARIANT 43: note = X may be any amino acid, preferably Q or K, more preferably Q
  VARIANT 48: note = X may be any amino acid, preferably M or I, more preferably M
  VARIANT 52: note = X may be any amino acid, preferably Y or N, more preferably Y
  VARIANT 54: note = X may be any amino acid, preferably G or S, more preferably S
  VARIANT 57: note = X may be any amino acid, preferably S or T, more preferably T
  VARIANT 61: note = X may be any amino acid, preferably D or A, more preferably A
  VARIANT 62: note = X may be any amino acid, preferably E or Q, more preferably Q
  VARIANT 65: note = X may be any amino acid, preferably K or Q, more preferably Q
  VARIANT 66: note = X may be any amino acid, preferably S or G, more preferably G
  VARIANT 67: note = X may be any amino acid, preferably R or K, more preferably R
  VARIANT 68: note = X may be any amino acid, preferably V or A, more preferably V
  VARIANT 70: note = X may be any amino acid, preferably M, L or I, more preferably M
  VARIANT 72: note = X may be any amino acid, preferably V, A or R, more preferably V
  VARIANT 74: note = X may be any amino acid, preferably K or T, more preferably K
  VARIANT 75: note = X may be any amino acid, preferably S or A, more preferably S
  VARIANT 76: note = X may be any amino acid, preferably T, K, I or S, more preferably T
  VARIANT 77: note = X may be any amino acid, preferably S or D, more preferably S
  VARIANT 79: note = X may be any amino acid, preferably A, L or V , more preferably A
  VARIANT 82: note = X may be any amino acid, preferably E or Q, more preferably E
  VARIANT 85: note = X may be any amino acid, preferably S or R, more preferably S
  VARIANT 87: note = X may be any amino acid, preferably R or T, more preferably R
  VARIANT 89: note = X may be any amino acid, preferably E or D, more preferably E
  VARIANT 91: note = X may be any amino acid, preferably T or S, more preferably T
  VARIANT 93: note = X may be any amino acid, preferably V or T, more preferably V
  VARIANT 97: note = X may be any amino acid, preferably A or T, more preferably A
  VARIANT 99: note = X may be any amino acid, preferably E or A, more preferably E
  VARIANT 100: note = X may be any amino acid, preferably R, K or A, more preferably R
  VARIANT 102: note = X may be any amino acid, preferably G or A, more preferably G
  VARIANT 104: note = X may be any amino acid, preferably N or A, more preferably N
  VARIANT 106: note = X may be any amino acid, preferably M or A, more preferably A
  VARIANT 107: note = X may be any amino acid, preferably D, E or A, more preferably E
  VARIANT 108: note = X may be any amino acid, preferably Y or A, more preferably Y
  VARIANT 114: note = X may be any amino acid, preferably L or S, more preferably L
QVQLXQXGXEXXXPGASVKXSCKASGYTFTXYWMDWVXQXPGXGLEWXGNIXPXSGXTNYXXKFXXXXTXTXDXXXXTXYMXLSXLXSXDXAXYYCXRXXYXRXAXXXWGQGTXVTVSS

SEQ ID NO: 213 - protein, 107 aa, synthetic construct
  VARIANT 9: note = X may be any amino acid, preferably S or A, more preferably S
  VARIANT 11: note = X may be any amino acid, preferably V, L or Q, more preferably V
  VARIANT 15: note = X may be any amino acid, preferably V or L, more preferably V
  VARIANT 17: note = X may be any amino acid, preferably D or E, more preferably D
  VARIANT 18: note = X may be any amino acid, preferably R or S, more preferably R
  VARIANT 24: note = X may be any amino acid, preferably L or R, more preferably R
  VARIANT 28: note = X may be any amino acid, preferably T, G or S, more preferably G
  VARIANT 30: note = X may be any amino acid, preferably G or S, more preferably G
  VARIANT 31: note = X may be any amino acid, preferably T or S, more preferably S
  VARIANT 32: note = X may be any amino acid, preferably W or Y, more preferably W
  VARIANT 43: note = X may be any amino acid, preferably A or S, more preferably A
  VARIANT 45: note = X may be any amino acid, preferably K or Q, more preferably K
  VARIANT 52: note = X may be any amino acid, preferably T or S, more preferably T
  VARIANT 55: note = X may be any amino acid, preferably A or Q, more preferably A
  VARIANT 56: note = X may be any amino acid, preferably D, T, S or E, more preferably D
  VARIANT 70: note = X may be any amino acid, preferably D or K, more preferably D
  VARIANT 72: note = X may be any amino acid, preferably T or S, more preferably T
  VARIANT 73: note = X may be any amino acid, preferably L or F, more preferably L
  VARIANT 74: note = X may be any amino acid, preferably T or K, more preferably T
  VARIANT 80: note = X may be any amino acid, preferably P or A, more preferably P
  VARIANT 84: note = X may be any amino acid, preferably A or V, more preferably A
  VARIANT 85: note = X may be any amino acid, preferably T or S, more preferably T
  VARIANT 91: note = X is L, A or S, preferably L
  VARIANT 94: note = X may be any amino acid, preferably T or F, more preferably T
DIQMTQSPXSXSASXGXXVTITCXASQXIXXXLAWYQQKPGKXPXLLIYAAXSLXXGVPSRFSGSGSGTXFXXXISSLQXEDFXXYYCQQXYSXPLTFGGGTKLEIK

SEQ ID NO: 214 - protein, 6 aa, synthetic construct
QTIGTY

SEQ ID NO: 215 - protein, 6 aa, synthetic construct
QTIGSW

SEQ ID NO: 216 - protein, 6 aa, synthetic construct
QTISTW

SEQ ID NO: 217 - protein, 6 aa, synthetic construct
QGIGTW

SEQ ID NO: 218 - protein, 6 aa, synthetic construct
QSIGTW

SEQ ID NO: 219 - protein, 5 aa, Homo sapiens
MYPPY

SEQ ID NO: 220 - protein, 382 aa, Homo sapiens
MRPGTALQAVLLAVLLVGLRAATGRLLSASDLDLRGGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 221 - protein, 229 aa, Homo sapiens
MVTSGLGSGGVRRNKAIAQPARTFMLGLMAAYHNLEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 222 - protein, 230 aa, Homo sapiens
MYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 223 - protein, 8 aa, synthetic construct
DYKDDDDK

SEQ ID NO: 224 - protein, 107 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKLEIK

SEQ ID NO: 225 - protein, 119 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARERYGRNAMEYWGQGTLVTVSS

SEQ ID NO: 226 - protein, 10 aa, Mus musculus
GYTFTSYWMD

SEQ ID NO: 227 - protein, 10 aa, synthetic construct
GYTFTGYWMD

SEQ ID NO: 228 - protein, 10 aa, synthetic construct
GYTFTSYWMH

SEQ ID NO: 229 - protein, 10 aa, synthetic construct
GYTFTSYYMD

SEQ ID NO: 230 - protein, 8 aa, Mus musculus
GYTFTSYW

SEQ ID NO: 231 - protein, 8 aa, synthetic construct
GYTFTGYW

SEQ ID NO: 232 - protein, 8 aa, synthetic construct
GYTFTSYY

SEQ ID NO: 233 - protein, 7 aa, Mus musculus
GYTFTSY

SEQ ID NO: 234 - protein, 7 aa, synthetic construct
GYTFTGY

SEQ ID NO: 235 - protein, 8 aa, synthetic construct
IYPSSGTT

SEQ ID NO: 236 - protein, 8 aa, synthetic construct
IYPGSGTT

SEQ ID NO: 237 - protein, 8 aa, Mus musculus
IYPGSGST

SEQ ID NO: 238 - protein, 8 aa, synthetic construct
IYPSSGST

SEQ ID NO: 239 - protein, 8 aa, synthetic construct
INPGSGST

SEQ ID NO: 240 - protein, 6 aa, synthetic construct
YPSSGT

SEQ ID NO: 241 - protein, 6 aa, synthetic construct
YPGSGT

SEQ ID NO: 242 - protein, 6 aa, Mus musculus
YPGSGS

SEQ ID NO: 243 - protein, 6 aa, synthetic construct
YPSSGS

SEQ ID NO: 244 - protein, 6 aa, synthetic construct
NPGSGS

SEQ ID NO: 245 - protein, 12 aa, synthetic construct
ARERYGRNAAEY

SEQ ID NO: 246 - protein, 12 aa, synthetic construct
TRERYGRNAAEY

SEQ ID NO: 247 - protein, 12 aa, synthetic construct
TREKYGRNAMEY

SEQ ID NO: 248 - protein, 12 aa, synthetic construct
TRERYGRNAMEY

SEQ ID NO: 249 - protein, 12 aa, synthetic construct
TRERYGRNAMEA

SEQ ID NO: 250 - protein, 12 aa, synthetic construct
TRERYGRNAMAY

SEQ ID NO: 251 - protein, 12 aa, synthetic construct
TRERYGRAAMEY

SEQ ID NO: 252 - protein, 12 aa, synthetic construct
TRERYARNAMEY

SEQ ID NO: 253 - protein, 12 aa, synthetic construct
TREAYGRNAMEY

SEQ ID NO: 254 - protein, 12 aa, synthetic construct
TRARYGRNAMEY

SEQ ID NO: 255 - protein, 12 aa, synthetic construct
ARERYGRNAMEY

SEQ ID NO: 256 - protein, 12 aa, Mus musculus
TRERYGRNAMDY

SEQ ID NO: 257 - protein, 12 aa, synthetic construct
TRERYGRQAMDY

SEQ ID NO: 258 - protein, 12 aa, synthetic construct
TRERYGANAMEY

SEQ ID NO: 259 - protein, 12 aa, synthetic construct
TRERAGRNAMEY

SEQ ID NO: 260 - protein, 8 aa, synthetic construct
RYGRNAAE

SEQ ID NO: 261 - protein, 8 aa, synthetic construct
KYGRNAME

SEQ ID NO: 262 - protein, 8 aa, synthetic construct
RYGRNAME

SEQ ID NO: 263 - protein, 8 aa, synthetic construct
RYGRNAMA

SEQ ID NO: 264 - protein, 8 aa, synthetic construct
RYGRAAME

SEQ ID NO: 265 - protein, 8 aa, synthetic construct
RYARNAME

SEQ ID NO: 266 - protein, 8 aa, synthetic construct
AYGRNAME

SEQ ID NO: 267 - protein, 8 aa, Mus musculus
RYGRNAMD

SEQ ID NO: 268 - protein, 8 aa, synthetic construct
RYGRQAMD

SEQ ID NO: 269 - protein, 8 aa, synthetic construct
RYGANAME

SEQ ID NO: 270 - protein, 8 aa, synthetic construct
RAGRNAME

SEQ ID NO: 271 - protein, 6 aa, synthetic construct
QGIGSW

SEQ ID NO: 272 - protein, 6 aa, Mus musculus
QTIGTW